(19) United States
(12) Patent Application Publication
(10) Pub. No.: US 2021/0094919 A1
(43) Pub. Date: Apr. 1, 2021

(54) KRAS MUTANT PROTEIN INHIBITORS

(71) Applicant: Jacobio Pharmaceuticals Co., Ltd., Beijing (CN)

(72) Inventors: Amin LI, Beijing (CN); Sujing LI, Beijing (CN); Peng WANG, Beijing (CN); Chaojie DANG, Beijing (CN); Dan LIU, Beijing (CN)

(73) Assignee: Jacobio Pharmaceuticals Co., Ltd.

(21) Appl. No.: 17/030,626

(22) Filed: Sep. 24, 2020

(30) Foreign Application Priority Data
Sep. 25, 2019 (CN) PCT/CN2019/107667
Sep. 30, 2019 (CN) PCT/CN2019/109303

(51) Int. Cl. C07D 221/04

(52) U.S. Cl. CPC C07D 221/04

(57) ABSTRACT

The invention relates to a KRAS mutant protein inhibitor, a composition containing the inhibitor and the use thereof.

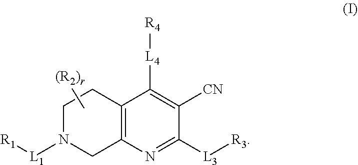

### TECHNICAL FIELD

The invention relates to a KRAS mutant protein inhibitor, a composition containing the inhibitor and the use thereof.

### BACKGROUND ART

RAS represents a population of 189 amino acid monomeric globular proteins (21 kDa molecular weight) that are associated with the plasma membrane and bind to GDP or GTP, and RAS acts as a molecular switch. When the RAS contains bound GDP, it is in a stationary or closed position and is “Mactive”. When cells are exposed to certain growth-promoting stimuli, RAS is induced to exchange their bound GDP for GTP. In the case of binding to GTP, RAS is “opened” and is capable of interacting with other proteins (its “downstream targets”) and activating the proteins. The RAS protein itself has an inherently low ability to hydrolyze GTP back to GDP, thereby turning itself into a closed state. Closing RAS requires an exogenous protein called GTPase activating protein (GAP) that interacts with RAS and greatly accelerates the conversion of GTP to GDP. Any mutation in RAS that affects its ability to interact with GAP or convert GTP back to GDP will result in prolonged protein activation, and thus conduction to the cell to inform its signalling of continued growth and division. Since these signals cause cell growth and division, over-activated RAS signaling can ultimately lead to cancer.

Structurally, the RAS protein contains a G domain responsible for the enzymatic activity of RAS, guanine nucleotide binding and hydrolysis (GTPase reaction). It also contains a C-terminal extension called the CAAX cassette, which can be post-translationally modified and responsible for targeting the protein to the membrane. The G domain is approximately 21-25 kDa in size and contains a phosphate binding ring (P-ring). The P-loop represents a pocket of binding nucleotide in protein, and this is a rigid portion of a domain with conserved amino acid residues necessary for nucleotide binding and hydrolysis (glycine 12, sulfo-aminolactic acid 26 and lysine 16). The G domain also contains a so-called switch I region (residues 30-40) and a switch II region (residues 60-76), both of which are dynamic parts of the protein, since the dynamic portion is converted between stationary and loaded states. The ability is often expressed as a “spring loaded” mechanism. The primary interaction is the hydrogen bond formed by threonine-35 and glycine-60 with the gamma-phosphate of GTP, which maintains the active conformation of the switch 1 region and the switch 2 regions, respectively. After hydrolysis of GTP and release of phosphate, the two relax into an inactive GDP conformation.

The most notable members of the RAS subfamily are HRAS, KRAS and NRAS, which are primarily involved in many types of cancer. Mutation of any of the three major isoforms of the RAS gene (HRAS, NRAS or KRAS) is one of the most common events in human tumor formation. Approximately 30% of all tumors in human tumors were found to carry some mutations in the RAS gene. It is worth noting that KRAS mutations were detected in 25%-30% of tumors. In contrast, the rate of carcinogenic mutations in NRAS and HRAS family members was much lower (8% and 3%, respectively). The most common KRAS mutations were found at residues G12 and G13 in the P-loop as well as at residue Q61.

G12C is a frequently occurring KRAS gene mutation (glycine-12 is mutated to cysteine). This mutation has been found in about 13% of cancers, about 43% in lung cancer, and almost 100% in MYH-associated polyposis (familial colon cancer syndrome). However, targeting this gene with small molecules is a challenge.

Thus, despite advances in this field, there remains a need in the art for improved compounds and methods for treating cancer, such as by inhibiting KRAS, HRAS or NRAS. The present invention fulfills this need and provides other related advantages.

### SUMMARY OF INVENTION

In one aspect, there is provided a compound of formula (I), a stereoisomer thereof, an atropisomer thereof, a pharmaceutically acceptable salt thereof, a pharmaceutically acceptable salt of the stereoisomer thereof or a pharmaceutically acceptable salt of the atropisomer thereof:

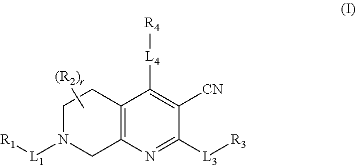

Wherein,

Each of L₁ at each occurrence is independently selected from absent, (CR₅R₆)ₘ, C(═O), O, NR₈, S, S(═O) or S(═O)₂;

Each of R₁ at each occurrence is independently selected from —C₁₋₆alkyl, —C₂₋₆alkenyl, —C₂₋₆alkynyl, —C₆₋₁₀ aryl, —C₁₋₆alkylene-C₆₋₁₀ aryl, 5-10 membered heteroaryl, —C₁₋₆alkylene-(5-10 membered heteroaryl), 3-6 membered heterocyclic, —C₁₋₆alkylene-(3-6 membered heterocyclic), —C₃₋₆carbocyclic, —C₁₋₆alkylene-C₃₋₆carbocyclic,

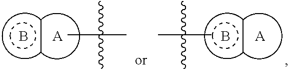

each of ring A at each occurrence is independently selected from a C₃₋₆carbocyclic or 3-6 membered heterocyclic ring, each of ring B at each occurrence is independently selected from a C₆₋₁₀ aryl or 5-10 membered heteroaryl ring, each of heterocyclic and heteroaryl at each occurrence independently contains 1, 2, 3 or 4 heteroatoms selected from N, O, S, S═O or S(═O)₂, each of which at each occurrence is independently optionally substituted by 1, 2, 3, 4, 5 or 6 R₁₁ or 1, 2, 3, 4, 5 or 6 R₁₂;

Each of R₁₁ at each occurrence is independently selected from —C₆₋₁₀aryl, —C₁₋₆alkylene-C₆₋₁₀aryl, 5-10 membered heteroaryl, —C₁₋₆alkylene-(5-10 membered heteroaryl), 3-6 membered heterocyclic, —C₁₋₆alkylene-(3-6 membered heterocyclic), —C₃₋₆carbocyclic, —C₁₋₆alkylene-C₃₋₆carbocyclic, each of which is independently optionally substituted by 1, 2, 3, 4, 5 or 6 R₁₂;

Each of R₁₂ at each occurrence is independently selected from halogen, oxo, —C₁₋₆alkyl, —C₂₋₆alkenyl, —C₂₋₆alkynyl, —C₁₋₆alkylene-(halo)₁₋₃, heteroC₂₋₆alkyl, —CN, —OR₈, —C₁₋₆alkylene-OR₈, —O—C₁₋₆alkylene-(halo)₁₋₃, —SR₈, —S—C₁₋₆alkylene-(halo)₁₋₃, —NR₈R₉, —C₁₋₆alkylene-NR₈R₉, —C(═O)R₈, —C(═O)OR₈, —OC(═O)R₈, —C(═O)NR₈R₉, —NR₈C(═O)R₈, —S(═O)₂NR₈R₉, —C₃₋₆carbocyclic, 3-6 membered heterocyclic, —C₆₋₁₀aryl, or 5-10 membered heteroaryl, each of heterocyclic and heteroaryl at each occurrence independently contains 1, 2, 3 or 4 heteroatoms selected from N, O, S, S═O or S(═O)₂, each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₂ at each occurrence is independently selected from halogen, —C₁₋₆alkyl, —C₁₋₆alkylene-(halo)₁₋₃, heteroC₂₋₆alkyl, —CN, —OR₈, —C₁₋₆alkylene-(OR₈)₁₋₃, —NR₈R₉, —C₁₋₆alkylene-NR₈R₉, —C(═O)R₈, —C(═O)OR₈, —OC(═O)R₈, —C(═O)NR₈R₉, —NR₈C(═O)R₈, —S(═O)₂NR₈R₉ or —C₃₋₆carbocyclic, each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of L₃ at each occurrence is independently selected from absent, (CR₅R₆)ₘ, C(═O), O, NR₈, S, S(═O) or S(═O)₂;

Each of R₃ at each occurrence is independently selected from hydrogen, halogen, —C₁₋₆alkyl, —C₂₋₆alkenyl, —C₂₋₆alkynyl, —C₁₋₆alkylene-(halo)₁₋₃, heteroC₂₋₆alkyl, —CN, —C₁₋₆alkylene-CN, —OR₈, —C₁₋₆alkylene-OR₈, —NR₈R₉, —C₁₋₆alkylene-NR₈R₉, —C(═O)R₈, —C₁₋₆alkylene-C(═O)R₈, —C(═O)OR₈, —C₁₋₆alkylene-C(═O)OR₈, —OC(═O)R₈, —C₁₋₆alkylene-OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —SO₂R₈, —C₁₋₆alkylene-SO₂R₈, —S(═O)₂NR₈R₉, —C₁₋₆alkylene-S(═O)₂NR₈R₉, —PO(R₈)₂, —C₁₋₆alkylene-PO(R₈)₂, —NR₈SO₂R₉, —C₁₋₆alkylene-NR₈SO₂R₉, —C₃₋₁₀carbocyclic, —C₁₋₆alkylene-C₃₋₁₀carbocyclic, 3-10 membered heterocyclic, —C₁₋₆alkylene-(3-10 membered heterocyclic), —C₆₋₁₀ aryl, —C₁₋₆alkylene-C₆₋₁₀aryl, 5-10 membered heteroaryl, —C₁₋₆alkylene-(5-10 membered hetero aryl), —C₁₋₆alkylene-O—C₁₋₆alkylene-(5-10 membered heteroaryl),

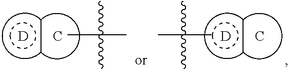

each of ring C at each occurrence is independently selected from a C₃₋₆ carbocyclic or 3-6 membered heterocyclic ring, each of ring D at each occurrence is independently selected from a C₆₋₁₀ aryl or 5-10 membered heteroaryl ring, each of heterocyclic and heteroaryl at each occurrence independently contains 1, 2, 3 or 4 heteroatoms selected from N, O, S, S═O or S(═O)₂, each of which at each occurrence is independently optionally substituted by 1, 2, 3, 4, 5 or 6 R₃₁;

Each of R₃, at each occurrence is independently selected from halogen, oxo, —C₁₋₆alkyl, —C₂₋₆alkenyl, —C₂₋₆alkynyl, —C₁₋₆alkylene-(halo)₁₋₃, heteroC₂₋₆alkyl, —CN, —C₁₋₆alkylene-CN, —OR₈, —C₁₋₆alkylene-OR₈, —O—C₁₋₆alkylene-(halo)₁₋₃, —NR₈R₉, —C₁₋₆alkylene-NR₈R₉, —O—C₁₋₆alkylene-NR₈R₉, —C(═O)R₈, —C₁₋₆alkylene-C(═O)R₈, —C(═O)OR₈, —C₁₋₆alkylene-C(═O)OR₈, —OC(═O)R₈, —C₁₋₆alkylene-OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —SO₂R₈, —C₁₋₆alkylene-SO₂R₈, —S(═O)₂NR₈R₉, —C₁₋₆alkylene-S(═O)₂NR₈R₉, —PO(R₈)₂, —C₁₋₆alkylene-PO(R₈)₂, —C₃₋₆carbocyclic, —C₁₋₆alkylene-C₃₋₆carbocyclic, 3-6 membered heterocyclic, —C₁₋₆alkylene-(3-6 membered heterocyclic), —C₆₋₁₀aryl, —C₁₋₆alkylene-C₆₋₁₀aryl, 5-10 membered heteroaryl, —C₁₋₆alkylene-(5-10 membered heteroaryl) or —C₁₋₆alkylene-O—C₁₋₆alkylene-C₃₋₆carbocyclic, each of heterocyclic or hetero aryl at each occurrence independently contains 1, 2, 3 or 4 heteroatoms selected from N, O, S, S═O or S(═O)₂, each of which at each occurrence is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of L₄ at each occurrence is independently selected from absent, (CR₅R₆), C(═O), O, NR₈, S, S(═O) or S(═O)₂;

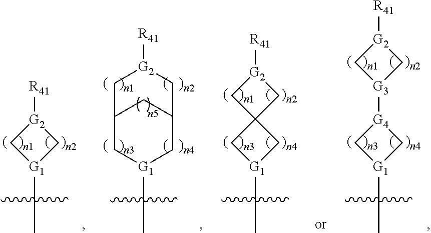

Each of R₄ at each occurrence is independently selected from each of

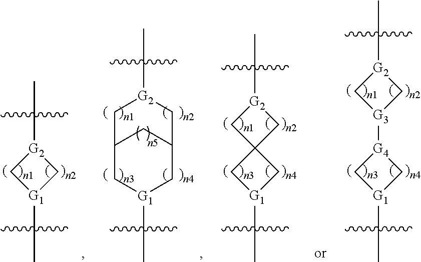

at each occurrence is independently optionally substituted by 1, 2, 3, 4, 5 or 6 R₄₂;

Each of G₁, G₂, G₃ and G₄ at each occurrence is independently selected from N or CR₅;

Each of n1, n2, n3, n4, n5 at each occurrence is independently selected from 0, 1, 2, 3, 4, 5 or 6, provided that n1 and n2 is not 0 at the same time, n3 and n4 is not 0 at the same time;

Each of R₄₁ at each occurrence is independently selected from

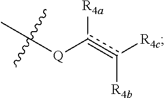

Each of Q at each occurrence is independently selected from C(═O), NR₈C(═O), S(═O)₂ or NR₈S(═O)₂;

[symbol] is selected from ═ or ≡;

Each of R₄ₐ, R_{4b} and R_{4c} at each occurrence is independently selected from absent, hydrogen, halogen, —C₁₋₆alkyl, —C₁₋₆alkylene-(halo)₁₋₃, heteroC₂₋₆alkyl, —CN, —OR₈, —C₁₋₆alkylene-OR₈, —NR₈R₉, —C₁₋₆alkylene-NR₈R₉, —NR₈—C₁₋₆alkylene-OR₈, —C(═O)R₈, —C(═O)OR₈, —OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —S(═O)₂NR₈R₉, —C₃₋₆carbocyclic, 3-6 membered heterocyclic or —C₁₋₆alkylene-(3-6 membered heterocyclic); each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

R_{4b} and R_{4c} together with the carbon which they both attach to form a C₃₋₁₀carbocyclic ring or a 3-10 membered heterocyclic ring, or R₄ₐ and R_{4b} with the carbon they respectively attach to form a C₃₋₆carbocyclic ring or a 3-6 membered heterocyclic ring, each of heterocyclic at each occurrence contains 1, 2 or 3 heteroatoms selected from N, O, S, S═O or S(═O)₂ and the carbocyclic or heterocyclic ring may be substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉ when [symbol] is selected from ═, or

Each of R₄ₐ is absent and one of R_{4b} and R_{4c} is absent, another of R_{4b} and R_{4c} is selected from hydrogen, halogen, —C₁₋₆alkyl, —C₁₋₆alkylene-(halo)₁₋₃, heteroC₂₋₆alkyl, —CN, —OR₈, —C₁₋₆alkylene-OR₈, —NR₈R₉, —C₁₋₆alkylene-NR₈R₉, —C(═O)R₈, —C(═O)OR₈, —OC(═O)R₈, —C(═O)NR₈R₉, —C₁₋₆alkylene-C(═O)NR₈R₉, —NR₈C(═O)R₈, —C₁₋₆alkylene-NR₈C(═O)R₈, —S(═O)₂NR₈R₉ or —C₃₋₁₀carbocyclic; each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉ when [symbol] is selected from ≡;

Each of R₄₂ at each occurrence is independently selected from halogen, oxo, —C₁₋₆alkyl, —C₁₋₆alkylene-(halo)₁₋₃, heteroC₂₋₆alkyl, —C₂₋₆alkenyl, —C₂₋₆alkynyl, —OR₈, —C₁₋₆alkylene-OR₈, —NR₈R₉, —C₁₋₆alkylene-NR₈R₉, —CN, —C₁₋₆alkylene-CN, —C(═O)R₈, —C₁₋₆alkylene-C(═O)R₈, —C(═O)OR₈, —C₁₋₆alkylene-C(═O)OR₈, —OC(═O)R₈, —C₁₋₆alkylene-OC(═O) R₈, —C(═O)NR₈R₉, —C₁₋₆alkylene-C(═O)NR₈R₉, —NR₈C(═O)R₈, —C₁₋₆alkylene-NR₈C(═O)R₈, —S(═O)₂NR₈R₉, —C₁₋₆alkylene-S(═O)₂NR₈R₉ or —C₃₋₆carbocyclic; each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

Two R₄₂ together with the atom which they both or respectively attach to form a C₃₋₆carbocyclic or 3-6 membered heterocyclic ring, each of the heterocyclic ring independently contains 1, 2 or 3 heteroatoms selected from N or O, each of which is independently optionally substituted by 1, 2, 3, 4, 5 or 6 substituents selected from halogen, —C₁₋₆alkyl, —C₁₋₆alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₅ and R₆ at each occurrence is independently selected from hydrogen, halogen, —C₁₋₆alkyl, —C₂₋₆alkenyl, —C₂₋₆alkynyl, oxo, —OR₈, —NR₈R₉, —CN, —C(═O)R₈, —C(═O)OR₈, —OC(═O)R₈, —C(═O)NR₈R₉, —NR₈C(═O)R₈, —S(═O)₂NR₈R₉ or —C₃₋₆carbocyclic; each of which at each occurrence is independently optionally substituted by 1, 2, 3, 4, 5 or 6 substituents selected from halogen, oxo, —C₁₋₆alkyl, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ at each occurrence is independently selected from hydrogen, —C₁₋₆alkyl or —C₃₋₆carbocyclic;

m is selected from 0, 1, 2, 3, 4, 5 or 6;

r is selected from 0, 1, 2, 3, 4, 5 or 6.

In some embodiments, each of L₁ at each occurrence is independently selected from absent or (CR₅R₆)ₘ;

Each of R₅ and R₆ in L₁ at each occurrence is independently selected from hydrogen or methyl, ethyl, propyl or isopropyl;

m in L₁ is selected from 1, 2 or 3.

In some embodiments, each of L₁ at each occurrence is independently selected from absent.

In some embodiments, each of R₁ at each occurrence is independently selected from —C₁₋₃alkyl, —C₂₋₃alkenyl, —C₂₋₃alkynyl, —C₆₋₁₀ aryl, —C₁₋₃alkylene-C₆₋₁₀ aryl, 5-10 membered heteroaryl, —C₁₋₃alkylene-(5-10 membered heteroaryl), 3-6 membered heterocyclic, —C₁₋₃alkylene-(3-6 membered heterocyclic), —C₃₋₆carbocyclic, —C₁₋₃alkylene-C₃₋₆carbocyclic,

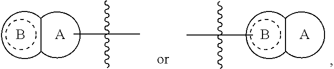

each of ring A at each occurrence is independently selected from a C₃₋₆carbocyclic or 3-6 membered heterocyclic ring, each of ring B at each occurrence is independently selected from a C₆₋₁₀ aryl or 5-10 membered heteroaryl ring, each of heterocyclic and heteroaryl at each occurrence independently contains 1, 2, 3 or 4 heteroatoms selected from N, O or S, each of which at each occurrence is independently optionally substituted by 1, 2, 3, 4, 5 or 6 R₁₁ or 1, 2, 3, 4, 5 or 6 R₁₂.

In some embodiments, each of R₁ at each occurrence is independently selected from methyl, ethyl, propyl, isopropyl, ethenyl, propenyl, ethynyl, propynyl, phenyl, naphthyl, -methylene-C₆₋₁₀aryl, -ethylene-C₆₋₁₀aryl, -propylene-C₆₋₁₀aryl, -isopropylene-C₆₋₁₀aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -isopropylene-(5-10 membered heteroaryl), 3 membered heterocyclic, 4 membered heterocyclic, 5 membered heterocyclic, 6 membered heterocyclic, -methylene-(3-6 membered heterocyclic), -ethylene-(3-6 membered heterocyclic), -propylene-(3-6 membered heterocyclic), -isopropylene-(3-6 membered heterocyclic), 3 membered carbocyclic, 4 membered carbocyclic, 5 membered carbocyclic, 6 membered carbocyclic, -methylene-C₃₋₆carbocyclic, -ethylene-C₃₋₆carbocyclic, -propylene-C₃₋₆carbocyclic, -isopropylene-C₃₋₆carbocyclic,

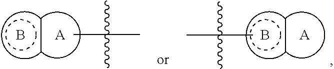

each of ring A at each occurrence is independently selected from a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring, each of ring B at each occurrence is independently selected from phenyl, naphthyl, 5 membered heteroaryl, 6 membered heteroaryl, 7 membered heteroaryl, 8 membered heteroaryl, 9 membered heteroaryl or 10 membered heteroaryl ring, each of heterocyclic and heteroaryl at each occurrence independently contains 1, 2, 3 or 4 heteroatoms selected from N, O or S, each of which at each occurrence is independently optionally substituted by 1, 2, 3, 4, 5 or 6 R₁₁ or 1, 2, 3, 4, 5 or 6 R₁₂.

In some embodiments, each of R₁ at each occurrence is independently selected from phenyl, naphthyl, 5 membered heteroaryl, 6 membered heteroaryl, 7 membered heteroaryl, 8 membered heteroaryl, 9 membered heteroaryl, 10 membered heteroaryl or

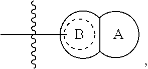

each of ring A at each occurrence is independently selected from a 5 membered carbocyclic, 6 membered carbocyclic, 5 membered heterocyclic or 6 membered heterocyclic ring, each of ring B at each occurrence is independently selected from a phenyl, 5 membered heteroaryl, or 6 membered heteroaryl ring, each of heteroaryl at each occurrence independently contains 1, 2, 3 or 4 heteroatoms selected from N, O or S; each of which at each occurrence is independently optionally substituted by 1, 2, 3, 4, 5 or 6 R₁₁ or 1, 2, 3, 4, 5 or 6 R₁₂. In some embodiments, each of R₁ at each occurrence is independently selected from phenyl, pyridyl, naphthyl, quinolyl, isoquinolyl, indolyl, indazolyl, benzo[d]imidazolyl, pyrazolo[3,4-b]pyridyl, 2,3-dihydrooxazolo[4,5-b]phenyl, 2,3-dihydrooxazolo[4,5-b]pyridyl or 1,2-dihydroisoquinoline, each of which at each occurrence is independently optionally substituted by 1, 2, 3, 4, 5 or 6 Ru or 1, 2, 3, 4, 5 or 6 R₁₂.

In some embodiments, each of R₁₁ at each occurrence is independently selected from —C₆₋₁₀aryl, —C₁₋₃alkylene-C₆₋₁₀aryl, 5-10 membered heteroaryl, —C₁₋₃alkylene-(5-10 membered heteroaryl), 3-6 membered heterocyclic, —C₁₋₃alkylene-(3-6 membered heterocyclic), —C₃₋₆carbocyclic or —C₁₋₃alkylene-C₃₋₆carbocyclic, each of heterocyclic and heteroaryl at each occurrence independently contains 1, 2 or 3 heteroatoms selected from N, O or S, each of which is independently optionally substituted by 1, 2, 3, 4, 5 or 6 R₁₂.

In some embodiments, each of R₁₁ at each occurrence is independently selected from phenyl, naphthyl, -methylene-C₆₋₁₀aryl, -ethylene-C₆₋₁₀aryl, -propylene-C₆₋₁₀aryl, -isopropylene-C₆₋₁₀aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -isopropylene-(5-10 membered heteroaryl), 3 membered heterocyclic, 4 membered heterocyclic, 5 membered heterocyclic, 6 membered heterocyclic, -methylene-(3-6 membered heterocyclic), -ethylene-(3-6 membered heterocyclic), -propylene-(3-6 membered heterocyclic), -isopropylene-(3-6 membered heterocyclic), 3 membered carbocyclic, 4 membered carbocyclic, 5 membered carbocyclic, 6 membered carbocyclic, -methylene-C₃₋₆carbocyclic, -ethylene-C₃₋₆carbocyclic, -propylene-C₃₋₆carbocyclic or -isopropylene-C₃₋₆carbocyclic, each of heterocyclic and heteroaryl at each occurrence independently contains 1, 2 or 3 heteroatoms selected from N or O; each of which at each occurrence is independently optionally substituted by 1, 2, 3, 4, 5 or 6 R₁₂.

In some embodiments, each of R₁₁ at each occurrence is independently selected from phenyl, naphthyl, 5 membered heteroaryl, 6 membered heteroaryl, 7 membered heteroaryl, 8 membered heteroaryl, 9 membered heteroaryl, 10 membered heteroaryl, 3 membered heterocyclic, 4 membered heterocyclic, 5 membered heterocyclic, 6 membered heterocyclic, 3 membered carbocyclic, 4 membered carbocyclic, 5 membered carbocyclic or 6 membered carbocyclic, each of heterocyclic and heteroaryl at each occurrence independently contains 1, 2 or 3 heteroatoms selected from N or O; each of which at each occurrence is independently optionally substituted by 1, 2, 3, 4, 5 or 6 R₁₂.

In some embodiments, each of R₁ is selected from:

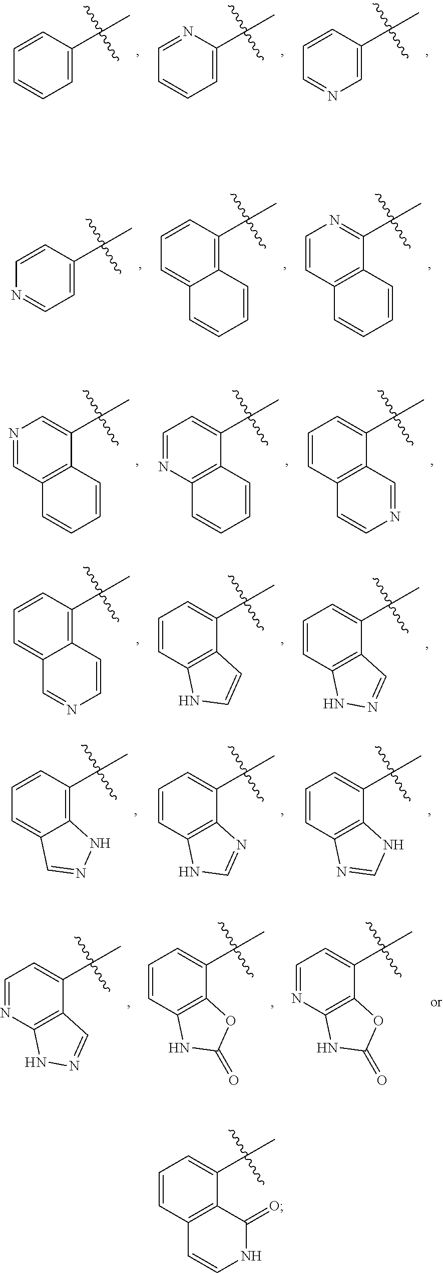

Each of which at each occurrence is independently optionally substituted by 1, 2, 3, 4, 5 or 6 R₁₂.

In some embodiments, each of R₁₂ at each occurrence is independently selected from —F, —Cl, —Br, oxo, —C₁₋₃alkyl, —C₂₋₃alkenyl, —C₂₋₃alkynyl, —C₁₋₃alkylene-(halo)₁₋₃, heteroC₂₋₃alkyl, —CN, —OR₈, —C₁₋₃alkylene-OR₈, —O—C₁₋₃alkylene-(halo)₁₋₃, —SR₈, —S—C₁₋₃alkylene-(halo)₁₋₃, —NR₈R₉, —C₁₋₃alkylene-NR₈R₉, —C(═O)R₈, —C(═O)OR₈, —OC(═O)R₈, —C(═O)NR₈R₉, —NR₈C(═O)R₈, —S(═O)₂NR₈R₉, —C₃₋₆carbocyclic, 3-6 membered heterocyclic, phenyl or 5-6 membered heteroaryl; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₁₂ at each occurrence is independently selected from hydrogen or —C₁₋₃alkyl.

In some embodiments, each of R₁₂ at each occurrence is independently selected from —F, —Cl, oxo, methyl, ethyl, propyl, isopropyl, ethenyl, propenyl, ethynyl, propynyl, -methylene-(halo)₁₋₃, -ethylene-(halo)₁₋₃-propylene-(halo)₁₋₃, heteroethyl, heteropropyl, —CN, —OR₈, -methylene-OR₈, -ethylene-OR₈, -propylene-OR₈, —O-methylene-(halo)₁₋₃, —O-ethylene-(halo)₁₋₃, —O-propylene-(halo)₁₋₃, —SR₈, —S-methylene-(halo)₁₋₃, —S-ethylene-(halo)₁₋₃, —S-propylene-(halo)₁₋₃, —NR₈R₉, -methylene-NR₈R₉, -ethylene-NR₈R₉, -propylene-NR₈R₉, —C(═O)R₈, —C(═O)OR₈, —OC(═O)R₈, —C(═O)NR₈R₉, —NR₈C(═O)R₈, —S(═O)₂NR₈R₉, 3 membered carbocyclic, 4 membered carbocyclic, 5 membered carbocyclic, 6 membered carbocyclic, 3 membered heterocyclic, 4 membered heterocyclic, 5 membered heterocyclic, 6 membered heterocyclic, phenyl, 5 membered heteroaryl or 6 membered heteroaryl, each of heterocyclic and heteroaryl at each occurrence is independently contains 1, 2 or 3 heteroatoms selected from N, O or S, each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₁₂ at each occurrence is independently selected from hydrogen, methyl, ethyl, propyl or isopropyl.

In some embodiments, each of R₁₂ at each occurrence is independently selected from —F, —Cl, oxo, methyl, ethyl, propyl, isopropyl, ethenyl, propenyl, ethynyl, propynyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —OCH₂F, —OCHF₂, —OCF₃, —OCH₂CH₂F, —OCH₂CHF₂, —OCH₂CF₃, —OCH₂CH₂CH₂F, —OCH₂CH₂CHF₂, —OCH₂CH₂CF₃, —SH, —SCH₃, —SCH₂CH₃, —SCH(CH₃)₂, —SCH₂F, —SCHF₂, —SCF₃, —SCH₂CH₂F, —SCH₂CHF₂, —SCH₂CF₃, —SCH₂CH₂CH₂F, —SCH₂CH₂CHF₂, —SCH₂CH₂CF₃, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂C H3, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃), —S(═O)₂N(CH₃)₂, 3 membered carbocyclic, 4 membered carbocyclic, 5 membered carbocyclic, 6 membered carbocyclic, 3 membered heterocyclic, 4 membered heterocyclic, 5 membered heterocyclic, 6 membered heterocyclic, phenyl, 5 membered heteroaryl or 6 membered heteroaryl, each of heterocyclic and heteroaryl at each occurrence is independently contains 1, or 2 heteroatoms selected from N or O, each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂.

In some embodiments, each of R₁₂ at each occurrence is independently selected from —F, —Cl, ═O, —CH₃, —CHF₂, —CF₃, —CN, —OH, —OCH₃, —OCF₃, —NH₂, —CONH₂, —SONH₂,

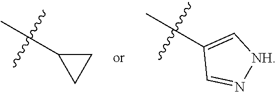

In some embodiments, each of R₁ is selected from:

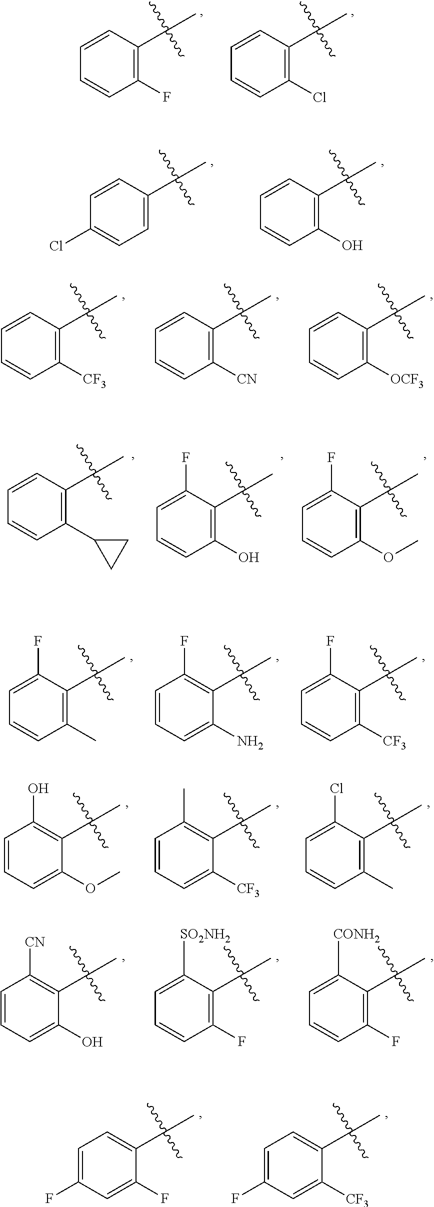

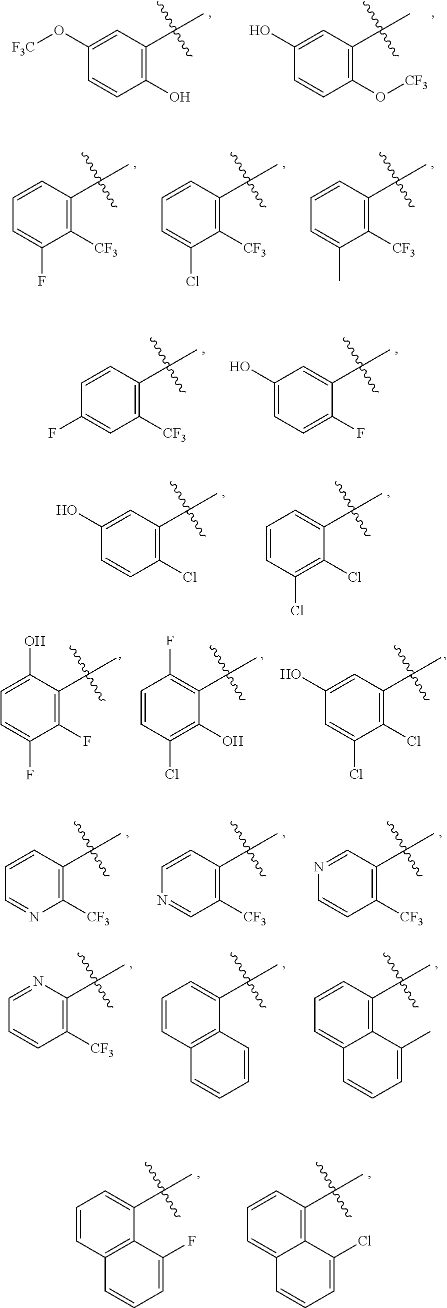

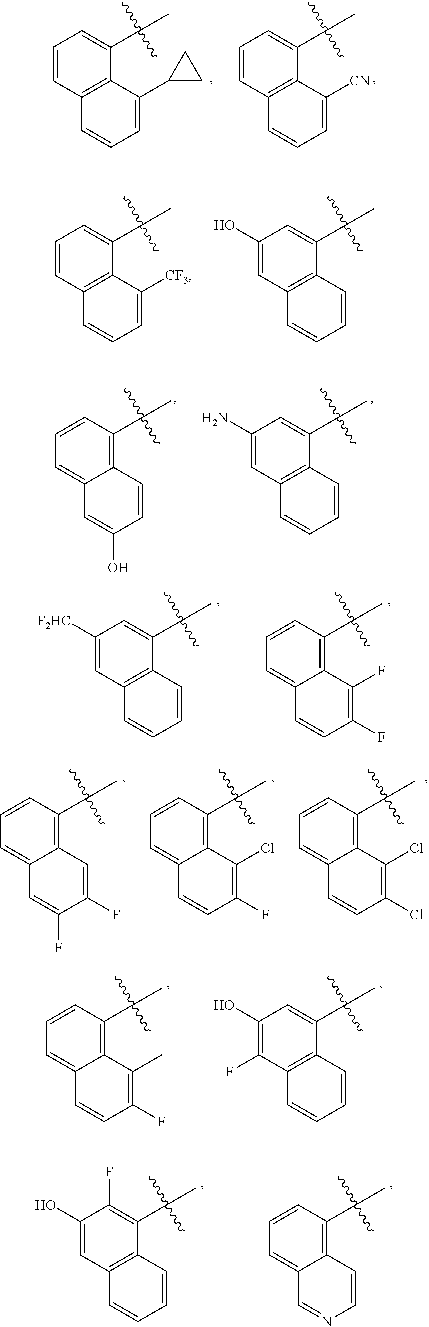

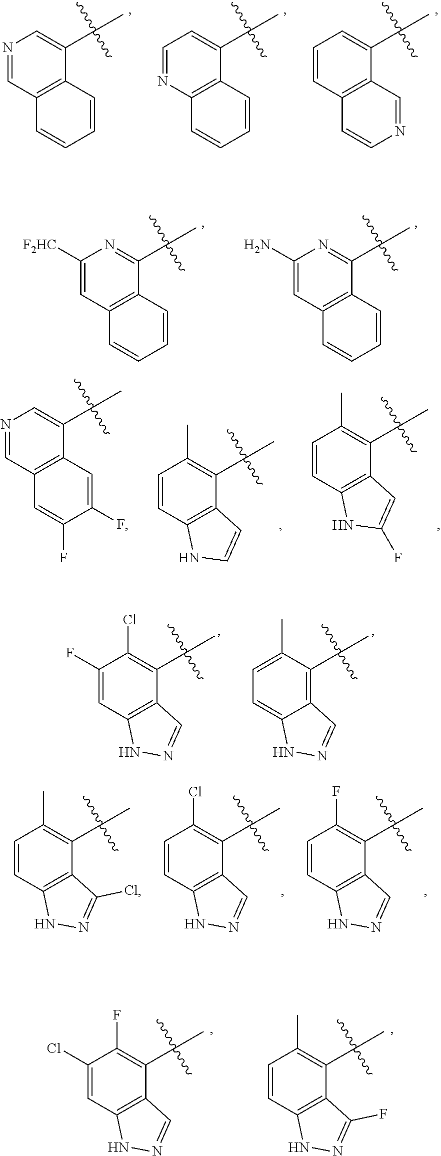

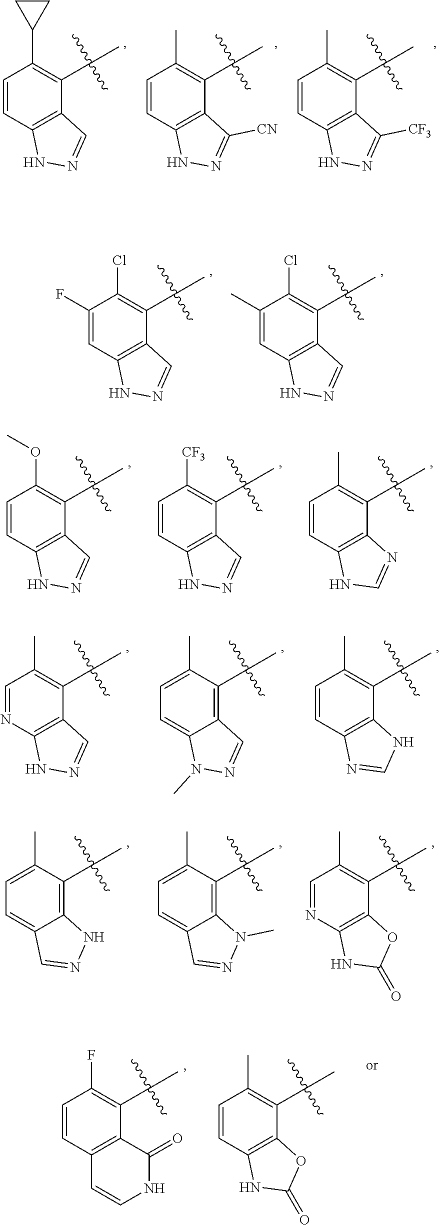

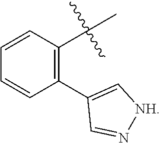 [SMILES: *c1ccccc1-c1cn[nH]c1]

In some embodiments, each of R₂ at each occurrence is independently selected from —F, —Cl, —Br, —C₁₋₃alkyl, —C₁₋₃alkylene-(halo)₁₃, heteroC₂₋₃alkyl, —CN, —OR₈, —C₁₋₃alkylene-(OR₈)₁₋₃, —NR₈R₉, —C₁₋₃alkylene-NR₈R₉, —C(═O)R₈, —C(═O)OR₈, —OC(═O)R₈, —C(═O)NR₈R₉, —NR₈C(═O)R₈, —S(═O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; Each of R₈ and R₉ in R₂ at each occurrence is independently selected from hydrogen or —C₁₋₃alkyl.

In some embodiments, each of R₂ at each occurrence is independently selected from —F, —Cl, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —CH₂CH₂CH₂OCH₃, —NH₂, —NHCH₃, —NHCH₂C H₃, —NHCH₂CH₂CH₃, —NHCH(CH₃)₂, —N(CH₃)₂, —N(CH₃)CH₂CH₃, —N(CH₃)CH₂CH₂C H₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃), —S(═O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, methyl, ethyl, propyl, isopropyl, methoxy, ethoxy, propoxy, isopropoxy, oxo, —OH, —NH₂, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂.

In some embodiments, each of R₂ at each occurrence is independently selected from hydrogen, —F, —Cl, methyl, —CH₂F, —CHF₂, —CF₃, —OH, —OCH₃, —CH₂OH, —NH₂, —NHCH₃ or —N(CH₃)₂.

In some embodiments, r is selected from 0, 1 or 2.

In some embodiments, r is selected from 0.

In some embodiments, each of L₃ at each occurrence is independently selected from absent, (CR₅R₆)ₘ, C(═O), 0, NR₈ or S;

Each of R₅ and R₆ in L₃ at each occurrence is independently selected from hydrogen or methyl, ethyl, propyl or isopropyl;

m in L₃ is selected from 1, 2 or 3;

R₈ in L₃ is selected from hydrogen or methyl.

In some embodiments, each of L₃ at each occurrence is independently selected from absent, C(═O), O, NH or S.

In some embodiments, each of R₃ at each occurrence is independently selected from hydrogen, —F, —Cl, —Br, —C₁₋₄alkyl, —C₂₋₄alkenyl, —C₂₋₄alkynyl, —C₁₋₄alkylene-(halo)₁₋₃, heteroC₂₋₄alkyl, —CN, —C₁₋₄alkylene-CN, —OR₈, —C₁₋₄alkylene-OR₈, —NR₈R₉, —C₁₋₄alkylene-NR₈R₉, —C(═O)R₈, —C₁₋₄alkylene-C(═O)R₈, —C(═O)OR₈, —C₁₋₄alkylene-C(═O)OR₈, —OC(═O)R₈, —C₁₋₄alkylene-OC(═O)R₈, —C(═O)NR₈R₉, —C₁₋₄alkylene-C(═O)NR₈R₉, —NR₈C(═O)R₈, —C₁₋₄alkylene-NR₈C(═O)R₈, —SO₂R₈, —C₁₋₄alkylene-SO₂R₈, —S(═O)₂NR₈R₉, —C₁₋₄alkylene-S(═O)₂NR₈R₉, —PO(R₈)₂, —C₁₋₄alkylene-PO(R₈)₂, —NR₈SO₂R₉, —C₁₋₄alkylene-NR₈SO₂R₉, —C₃₋₁₀carbocyclic, —C₁₋₄alkylene-C₃₋₁₀carbocyclic, 3-10 membered heterocyclic, —C₁₋₄alkylene-(3-10 membered heterocyclic), —C₆₋₁₀aryl, —C₁₋₄alkylene-C₆₋₁₀aryl, 5-10 membered heteroaryl, —C₁₋₄alkylene-(5-10 membered hetero aryl), —C₁₋₂alkylene-O—C₁₋₂alkylene-(5-10 membered heteroaryl),

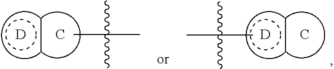

each of ring C at each occurrence is independently selected from a C₃₋₆ carbocyclic or 3-6 membered heterocyclic ring, each of ring D at each occurrence is independently selected from a phenyl or 5-6 membered heteroaryl ring, each of heterocyclic and heteroaryl at each occurrence independently contains 1, 2 or 3 heteroatoms selected from N, O, or S(═O)₂, each of which at each occurrence is independently optionally substituted by 1, 2, 3, 4, 5 or 6 R₃₁;

Each of R₈ or R₉ in R₃ at each occurrence is independently selected from hydrogen, —C₁₋₃alkyl or —C₃₋₆carbocyclic.

In some embodiments, each of R₃ at each occurrence is independently selected from hydrogen, —F, —Cl, methyl, ethyl, propyl, isopropyl, butyl, ethenyl, propenyl, butylenyl, ethynyl, propynyl, butynyl, -methylene-(halo)₁₋₃, -ethylene-(halo)₁₋₃, -propylene-(halo)₁₋₃, -butylene-(halo)₁₋₃, heteroethyl, heteropropyl, heterobutyl, —CN, -methylene-CN, -ethylene-CN, -propylene-CN, -butylene-CN, —OR₈, -methylene-OR₈, -ethylene-OR₈, -propylene-OR₈, -butylene-OR₈, —NR₈R₉, -methylene-NR₈R₉, -ethylene-NR₈R₉, -propylene-NR₈R₉, -butylene-NR₈R₉, —C(═O)R₈, -methylene-C(═O)R₈, -ethylene-C(═O)R₈, -propylene-C(═O)R₈, -butylene-C(═O)R₈, —C(═O)OR₈, -methylene-C(═O)OR₈, -ethylene-C(═O)OR₈, -propylene-C(═O)OR₈, -butylene-C(═O)OR₈, —OC(═O)R₈, -methylene-OC(═O)R₈, -ethylene-OC(═O)R₈, -propylene-OC(═O)R₈, -butylene-OC(═O)R₈, —C(═O)NR₈R₉, -methylene-C(═O)NR₈R₉, -ethylene-C(═O)NR₈R₉, -propylene-C(═O)NR₈R₉, -butylene-C(═O)NR₈R₉, —NR₈C(═O)R₈, -methylene-NR₈C(═O)R₈, -ethylene-NR₈C(═O)R₈, -propylene-NR₈C(═O)R₈, -butylene-NR₈C(═O)R₈, —SO₂R₈, -methylene-SO₂R₈, -ethylene-SO₂R₈, -propylene-SO₂R₈, -butylene-SO₂R₈, —S(═O)₂NR₈R₉, -methylene-S(═O)₂NR₈R₉, -ethylene-S(═O)₂NR₈R₉, -propylene-S(═O)₂NR₈R₉, -butylene-S(═O)₂NR₈R₉, —PO(R₈)₂, -methylene-PO(R₈)₂, -ethylene-PO(R₈)₂, -propylene-PO(R₈)₂, -butylene-PO(R₈)₂, —NR₈SO₂R₉, -methylene-NR₈SO₂R₉, -ethylene-NR₈SO₂R₉, -propylene-NR₈SO₂R₉, -butylene-NR₈SO₂R₉, 3 membered carbocyclic, 4 membered carbocyclic, 5 membered carbocyclic, 6 membered carbocyclic, 7 membered carbocyclic, 8 membered carbocyclic, 9 membered carbocyclic, 10 membered carbocyclic, -methylene-C₃₋₁₀carbocyclic, -ethylene-C₃₋₁₀carbocyclic, -propylene-C₃₋₁₀carbocyclic, -butylene-C₃₋₁₀carbocyclic, 3 membered heterocyclic, 4 membered heterocyclic, 5 membered heterocyclic, 6 membered heterocyclic, 7 membered heterocyclic, 8 membered heterocyclic, 9 membered heterocyclic, 10 membered heterocyclic, -methylene-(3-10 membered heterocyclic), -ethylene-(3-10 membered heterocyclic), -propylene-(3-10 membered heterocyclic), -butylene-(3-10 membered heterocyclic), phenyl, naphthyl, -methylene-C₆₋₁₀aryl, -ethylene-C₆₋₁₀aryl, -propylene-C₆₋₁₀aryl, -butylene-C₆₋₁₀aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -butylene-(5-10 membered heteroaryl), -methylene-O-methylene-(5-10 membered heteroaryl), -methylene-O-ethylene-(5-10 membered hetero aryl), -ethylene-O-methylene-(5-10 membered hetero aryl), -ethylene-O-ethylene-(5-10 membered heteroaryl),

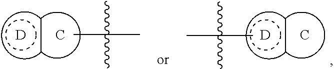

each of ring C at each occurrence is independently selected from a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring, each of ring D at each occurrence is independently selected from a phenyl, 5 membered heteroaryl or 6 membered heteroaryl ring, each of heterocyclic and heteroaryl at each occurrence independently contains 1, 2 or 3 heteroatoms selected from N, O, or S(═O)₂, each of which at each occurrence is independently optionally substituted by 1, 2, 3, 4, 5 or 6 R₃₁;

Each of R₈ or R₉ in R₃ at each occurrence is independently selected from hydrogen, methyl, ethyl, propyl, isopropyl, 3 membered carbocyclic, 4 membered carbocyclic, 5 membered carbocyclic or 6 membered carbocyclic.

In some embodiments, each of R₃ at each occurrence is independently selected from:

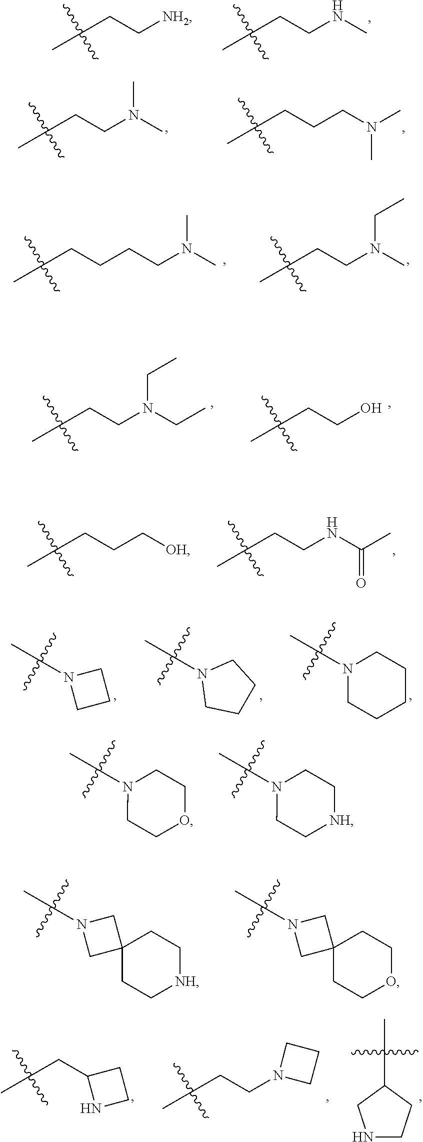

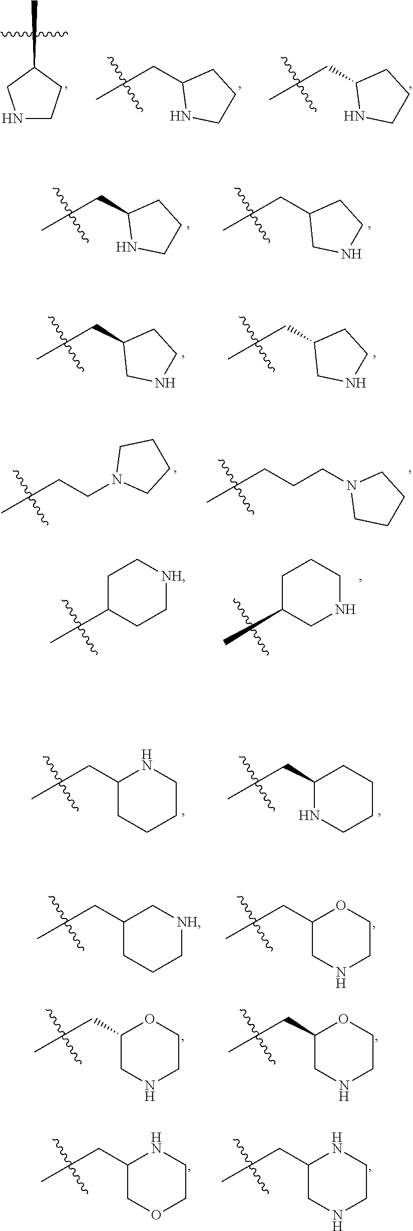

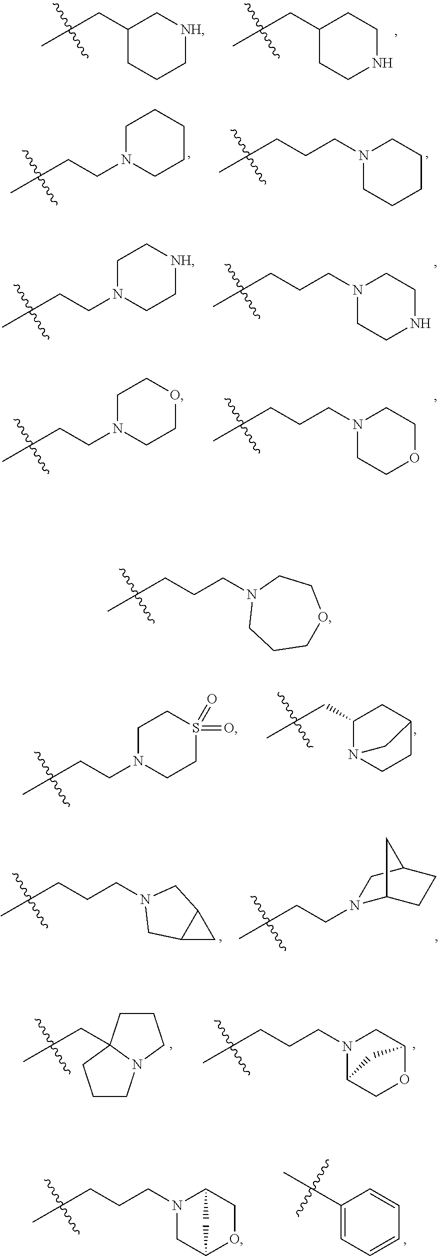

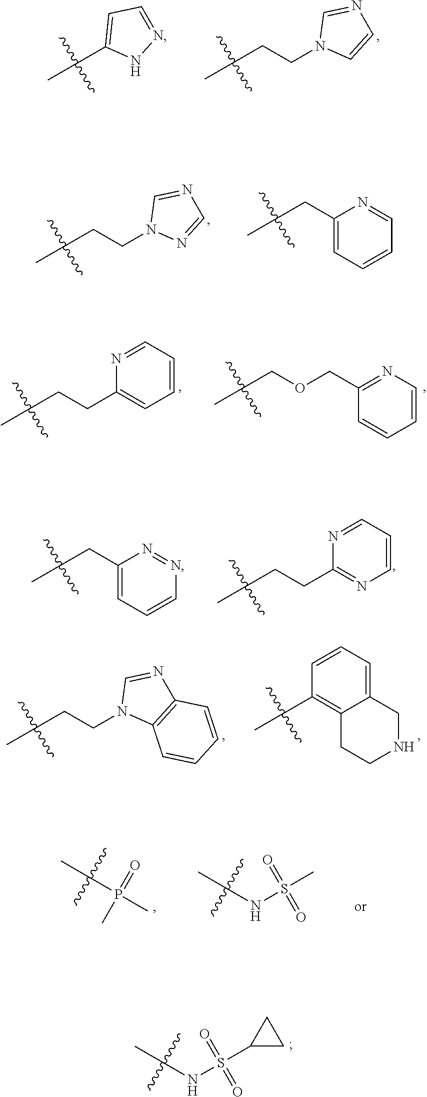

each of which at each occurrence is independently optionally substituted by 1, 2, 3, 4, 5 or 6 R₃₁.

In some embodiments, each of R₃₁ at each occurrence is independently selected from —F, —Cl, —Br, oxo, —C₁₋₄alkyl, —C₂₋₄alkenyl, —C₂₋₄alkynyl, —C₁₋₄alkylene-(halo)₁₋₃, heteroC₂₋₄alkyl, —CN, —C₁₋₄alkylene-CN, —OR₈, —C₁₋₄alkylene-OR₈, —O—C₁₋₄alkylene-(halo)₁₋₃, —NR₈R₉, —C₁₋₄alkylene-NR₈R₉, —O—C₁₋₄alkylene-NR₈R₉, —C(═O)R₈, —C₁₋₄alkylene-C(═O)R₈, —C(═O)OR₈, —C₁₋₄alkylene-C(═O)OR₈, —OC(═O)R₈, —C₁₋₄alkylene-OC(═O)R₈, —C(═O)NR₈R₉, —C₁₋₄alkylene-C(═O)NR₈R₉, —NR₈C(═O)R₈, —C₁₋₄alkylene-NR₈C(═O)R₈, —SO₂R₈, —C₁₋₄alkylene-SO₂R₈, —S(═O)₂NR₈R₉, —C₁₋₄alkylene-S(═O)₂NR₈R₉, —PO(R₈)₂, —C₁₋₄alkylene-PO(R₈)₂, —C₃₋₆carbocyclic, —C₁₋₄alkylene-C₃₋₆carbocyclic, 3-6 membered heterocyclic, —C₁₋₄alkylene-(3-6 membered heterocyclic), —C₆₋₁₀aryl, —C₁₋₄alkylene-C₆₋₁₀aryl, 5-10 membered heteroaryl, —C₁₋₄alkylene-(5-10 membered heteroaryl) or —C₁₋₂alkylene-O—C₁₋₂alkylene-C₃₋₆carbocyclic, each of heterocyclic or heteroaryl at each occurrence independently contains 1, 2 or 3 heteroatoms selected from N or O, each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₃, at each occurrence is independently selected from hydrogen or —C₁₋₃alkyl.

In some embodiments, each of R₃, at each occurrence is independently selected from —F, —Cl, oxo, methyl, ethyl, propyl, isopropyl, butyl, ethenyl, propenyl, butenyl, ethynyl, propynyl, butynyl, -methylene-(halo)₁₋₃, -ethylene-(halo)₁₋₃, -propylene-(halo)₁₋₃, -butylene-(halo)₁₋₃, heteroethyl, heteropropyl, heterobutyl, —CN, -methylene-CN, -ethylene-CN, -propylene-CN, -butylene-CN, —OR₈, -methylene-OR₈, -ethylene-OR₈, -propylene-OR₈, -butylene-OR₈, —O-methylene-(halo)₁₋₃, —O— ethylene-(halo)₁₋₃, —O-propylene-(halo)₁₋₃, —O-butylene-(halo)₁₋₃, —NR₈R₉, -methylene-NR₈R₉, -ethylene-NR₈R₉, -propylene-NR₈R₉, -butylene-NR₈R₉, —O-methylene-NR₈R₉, —O-ethylene-NR₈R₉, —O-propylene-NR₈R₉, —O-butylene-NR₈R₉, —C(═O)R₈, -methylene-C(═O)R₈, -ethylene-C(═O)R₈, -propylene-C(═O)R₈, -butylene-C(═O)R₈, —C(═O)OR₈, -methylene-C(═O)OR₈, -ethylene-C(═O)OR₈, -propylene-C(═O)OR₈, -butylene-C(═O)OR₈, —OC(═O)R₈, -methylene-OC(═O)R₈, -ethylene-OC(═O)R₈, -propylene-OC(═O)R₈, -butylene-OC(═O)R₈, —C(═O)NR₈R₉, -methylene-C(═O)NR₈R₉, -ethylene-C(═O)NR₈R₉, -propylene-C(═O)NR₈R₉, -butylene-C(═O)NR₈R₉, —NR₈C(═O)R₈, -methylene-NR₈C(═O)R₈, -ethylene-NR₈C(═O)R₈, -propylene-NR₈C(═O)R₈, -butylene-NR₈C(═O)R₈, —SO₂R₈, -methylene-SO₂R₈, -ethylene-SO₂R₈, -propylene-SO₂R₈, -butylene-SO₂R₈, —S(═O)₂NR₈R₉, -methylene-S(═O)₂NR₈R₉, -ethylene-S(═O)₂NR₈R₉, -propylene-S(═O)₂NR₈R₉, -butylene-S(═O)₂NR₈R₉, —PO(R₈)₂, -methylene-PO(R₈)₂, -ethylene-PO(R₈)₂, -propylene-PO(R₈)₂, -butylene-PO(R₈)₂, 3 membered carbocyclic, 4 membered carbocyclic, 5 membered carbocyclic, 6 membered carbocyclic, -methylene-C₃₋₆carbocyclic, -ethylene-C₃₋₆carbocyclic, -propylene-C₃₋₆carbocyclic, -butylene-C₃₋₆carbocyclic, 3 membered heterocyclic, 4 membered heterocyclic, 5 membered heterocyclic, 6 membered heterocyclic, -methylene-(3-6 membered heterocyclic), -ethylene-(3-6 membered heterocyclic), -propylene-(3-6 membered heterocyclic), -butylene-(3-6 membered heterocyclic), —C₆₋₁₀ aryl, -methylene-C₆₋₁₀aryl, -ethylene-C₆₋₁₀aryl, -propylene-C₆₋₁₀aryl, -butylene-C₆₋₁₀ aryl, 5 membered heteroaryl, 6 membered heteroaryl, 7 membered heteroaryl, 8 membered heteroaryl, 9 membered heteroaryl, 10 membered heteroaryl, -methylene-(5-10 membered heteroaryl), -ethylene-(5-10 membered heteroaryl), -propylene-(5-10 membered heteroaryl), -butylene-(5-10 membered heteroaryl), -methylene-O-methylene-C₃₋₆carbocyclic, -methylene-O-ethylene-C₃₋₆carbocyclic, -ethylene-O-methylene-C₃₋₆carbocyclic or -ethylene-O-ethylene-C₃₋₆carbocyclic; each of heterocyclic or heteroaryl at each occurrence independently contains 1 or 2 heteroatoms selected from N or O; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₃₁ at each occurrence is independently selected from hydrogen, methyl, ethyl, propyl or isopropyl.

In some embodiments, each of R₃₁ at each occurrence is independently selected from —F, —Cl, oxo, methyl, ethyl, propyl, isopropyl, butyl, ethenyl, propenyl, butenyl, ethynyl, propynyl, butynyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH(CH₃)(CF₃), —CH(CH₂F)₂, —CH₂OCH₃, —CH₂CH₂OCH₃, —CH₂CH₂CH₂OCH₃, —CN, —CH₂CN, —CH₂CH₂CN, —CH₂CH₂CH₂CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —CH₂C(CH₃)₂OH, —OCF₃, —OCH₂CF₃, —OCH₂CH₂CF₃, —NH₂, —NHCH₃, —NHCH₂CH₃, —NHCH₂CH₂CH₃, —NHCH(CH₃)₂, —N(CH₃)₂, —N(CD₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —OCH₂NH₂, —OCH₂CH₂NH₂, —OCH₂CH₂CH₂NH₂, —C(═O)CH₃, —CH₂C(═O)CH₃, —CH₂CH₂C(═O)CH₃, —CH₂CH₂CH₂C(═O)CH₃, —C(═O)OCH₃, —CH₂C(═O)OCH₃, —CH₂CH₂C(═O)OCH₃, —CH₂CH₂CH₂C(═O)OCH₃, —OC(═O)CH₃, —CH₂OC(═O)CH₃, —CH₂CH₂OC(═O)CH₃, —CH₂CH₂CH₂OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —SO₂CH₃, —CH₂SO₂CH₃, —CH₂CH₂SO₂CH₃, —CH₂CH₂CH₂SO₂CH₃, —S(═O)₂NH₂, —S(═O)₂NH(CH₃), —S(═O)₂N(CH₃)₂, —CH₂S(═O)₂N(CH₃)₂, —CH₂CH₂S(═O)₂N(CH₃)₂, —CH₂CH₂CH₂S(═O)₂N(CH₃)₂, —PO(CH₃)₂, —CH₂PO(CH₃)₂, —CH₂CH₂PO(CH₃)₂, —CH₂CH₂CH₂PO(CH₃)₂,

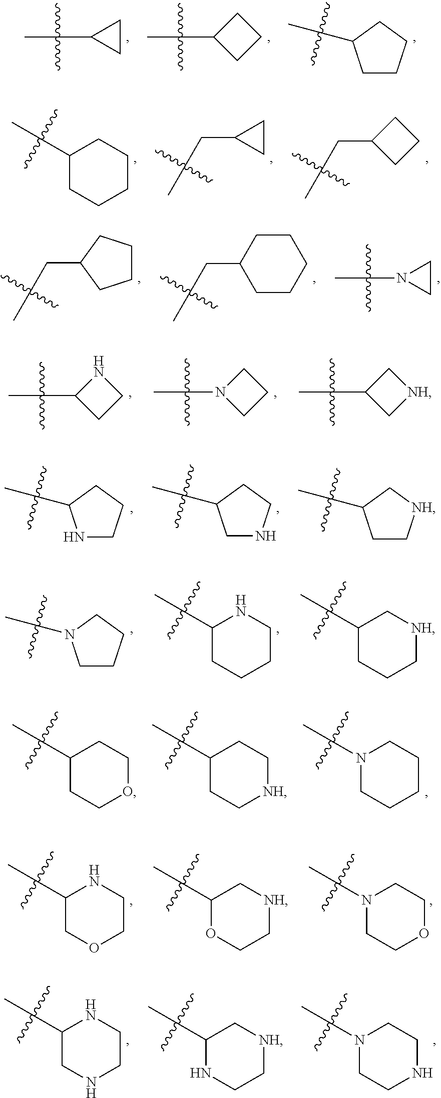

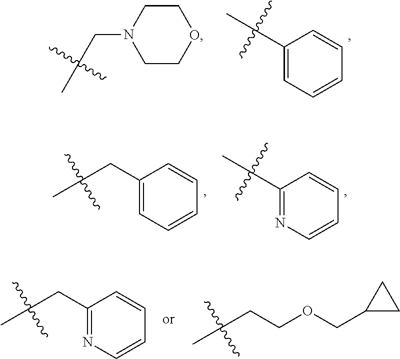 [SMILES: *CN1CCOCC1 ; *c1ccccc1 ; *Cc1ccccc1 ; *c1ccccn1 ; *Cc1ccccn1 ; *CCOCC1CC1]

each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂.

In some embodiments, each of R₃₁ at each occurrence is independently selected from —F, oxo, methyl, ethyl, isopropyl, —CH(CH₂F)₂, —CH₂OCH₃, —CH₂CH₂OCH₃, —OH, —CH₂OH, —CH₂CH₂OH, —CH₂C(CH₃)₂OH, —N(CH₃)₂, —N(CD₃)₂, —C(═O)CH₃, —CH₂C(═O)N(CH₃)₂,

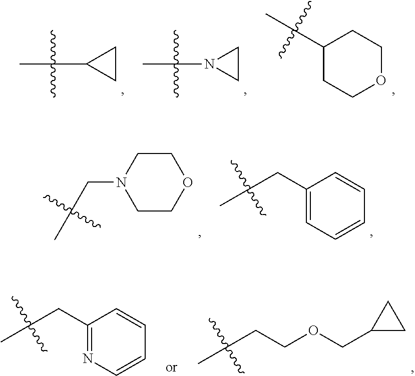

each of which is independently optionally substituted by 1, 2, 3, 4, 5 or 6 substituents selected from —F, methyl, ethyl, propyl, isopropyl, methoxy, ethoxy, propoxy, isopropoxy, oxo, —OH, —NH₂, —N(CH₃)₂ or —CN.

In some embodiments, each of R₃₁ at each occurrence is independently selected from —F, oxo, methyl, ethyl, isopropyl, —CH(CH₂F)₂, —CH₂OCH₃, —CH₂CH₂OCH₃, —OH, —CH₂OH, —CH₂CH₂OH, —CH₂C(CH₃)₂OH, —N(CH₃)₂, —N(CD₃)₂, —C(═O)CH₃, —CH₂C(═O)N(CH₃)₂,

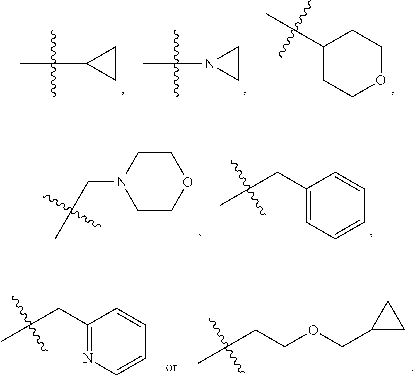

In some embodiments, each of R₃ at each occurrence is independently selected from:

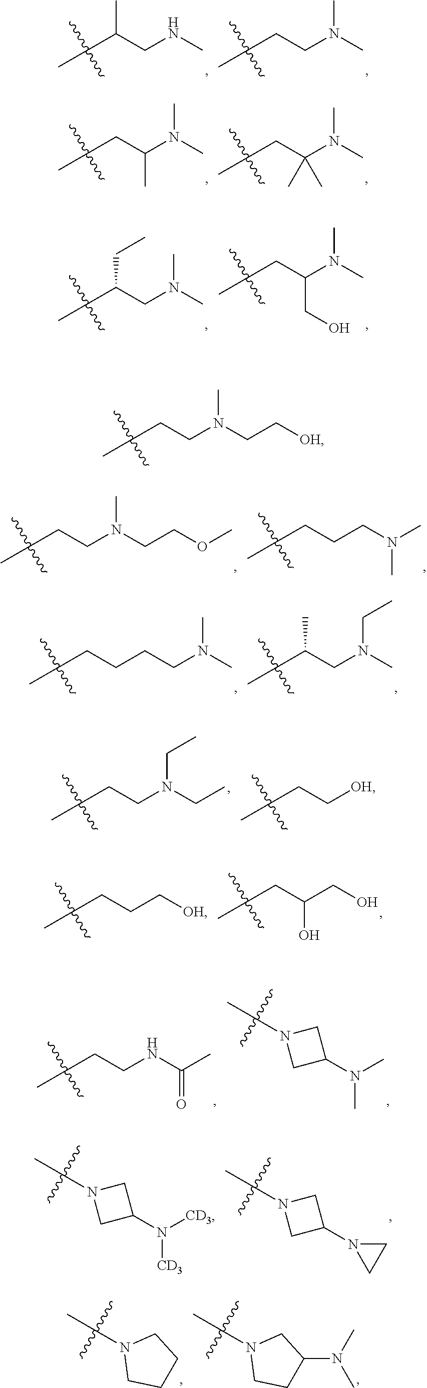

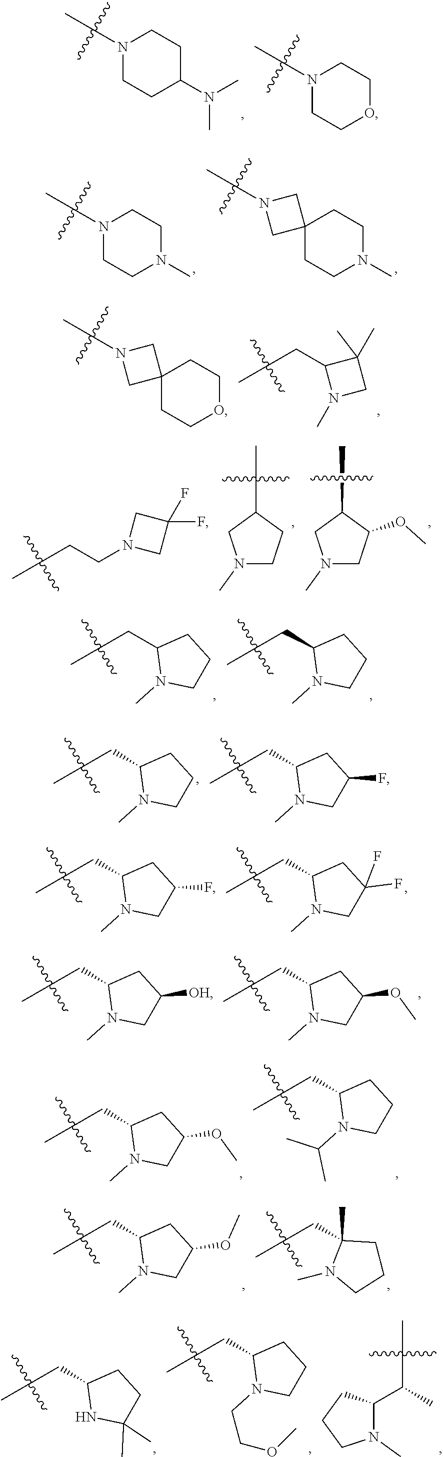

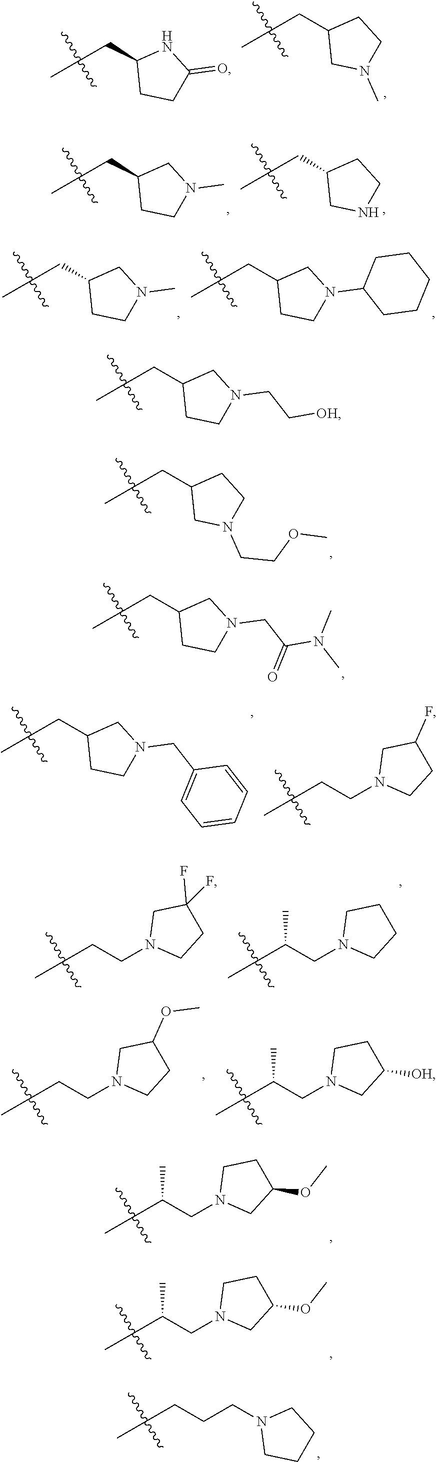

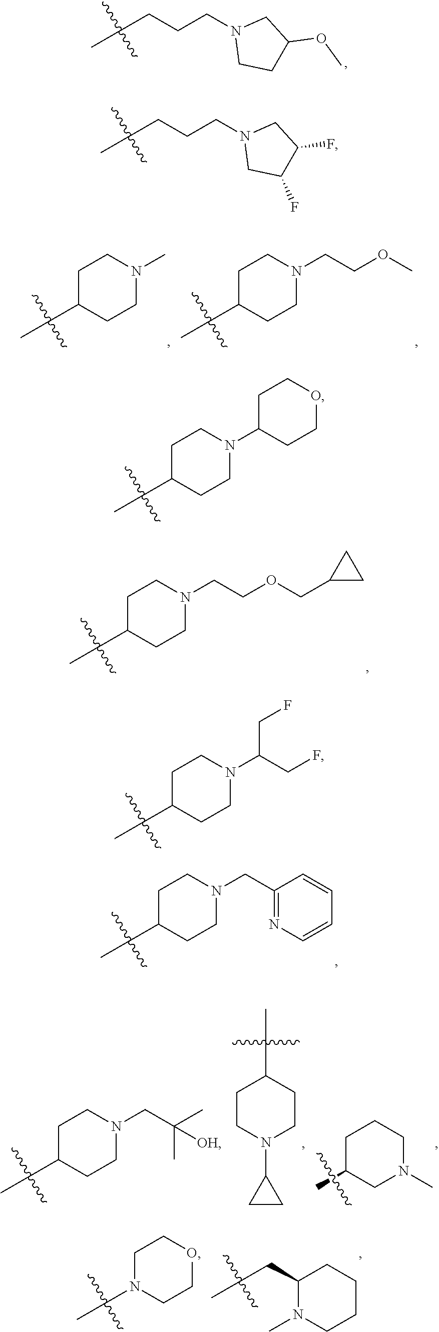

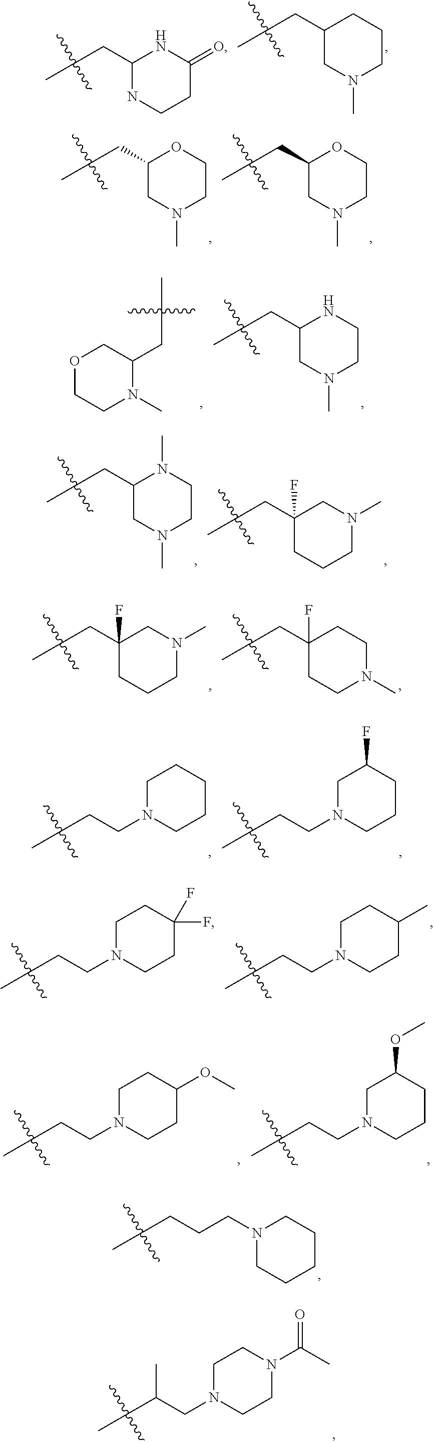

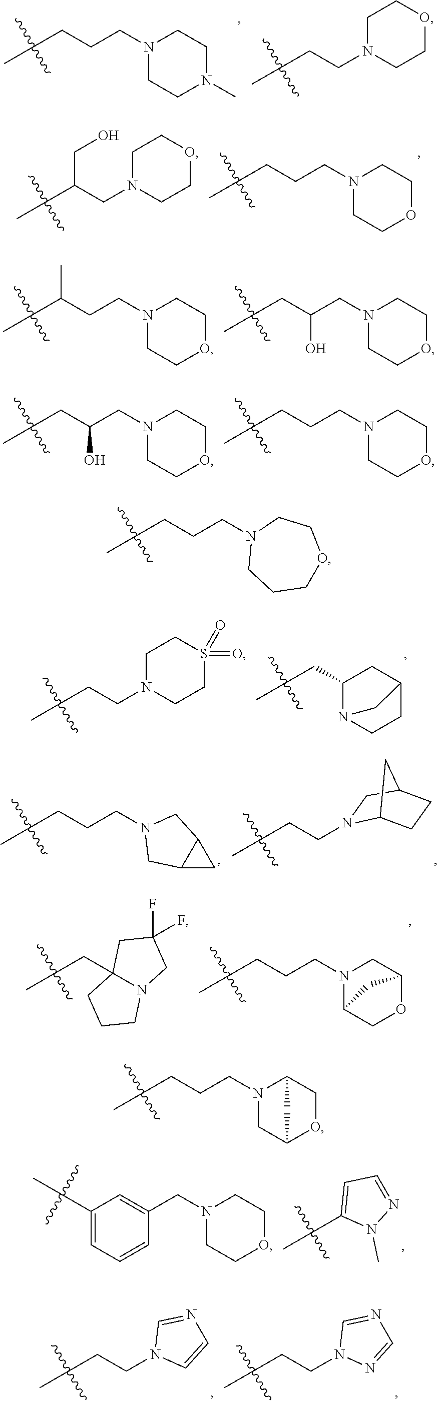

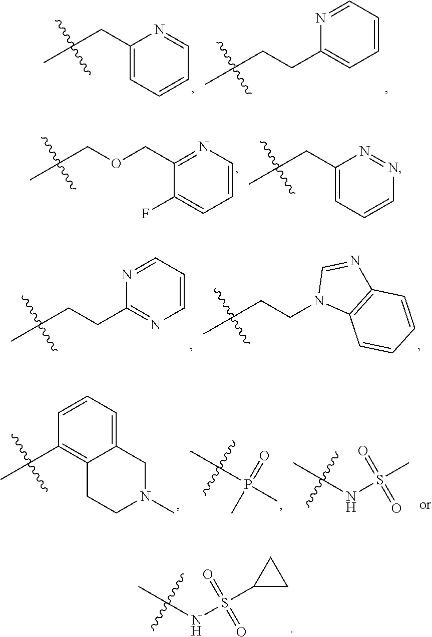

In some embodiments, each of R₃-L₃- at each occurrence is independently selected from:

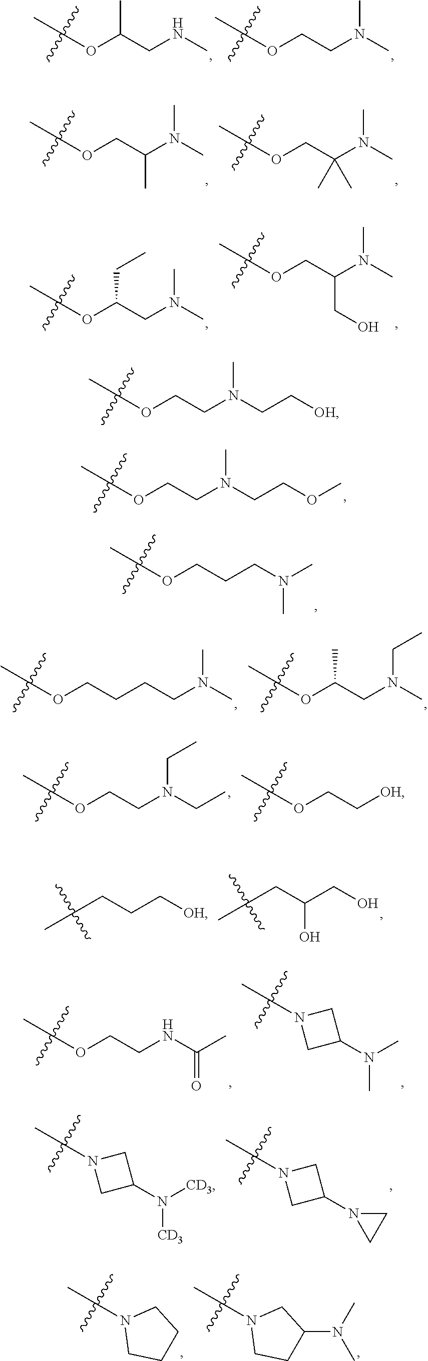

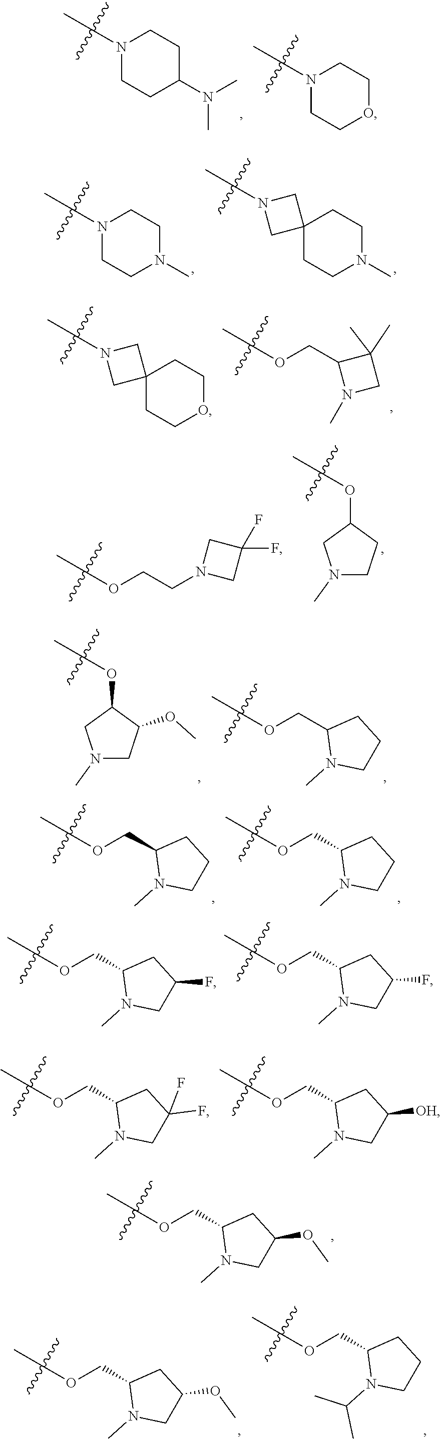

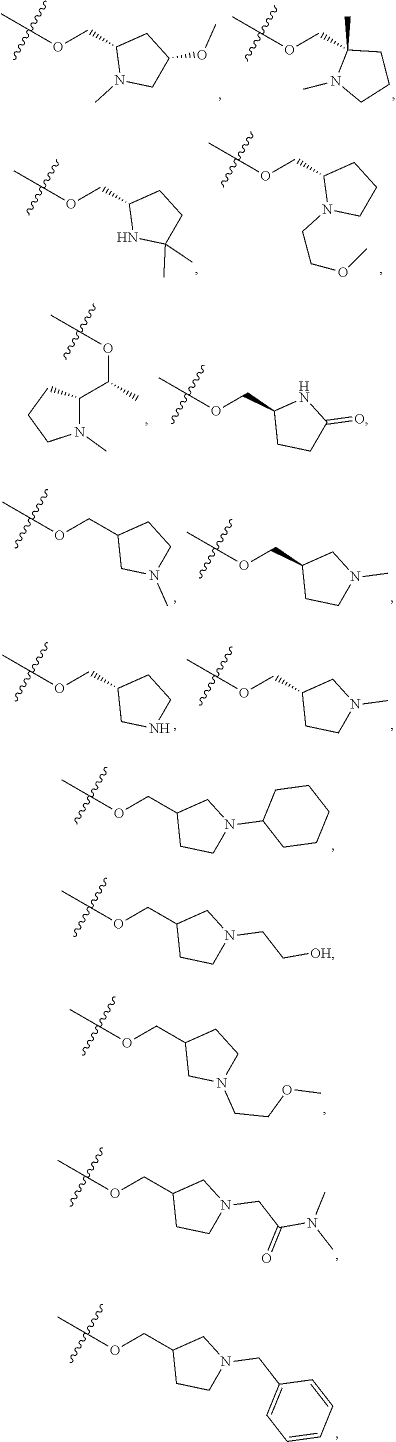

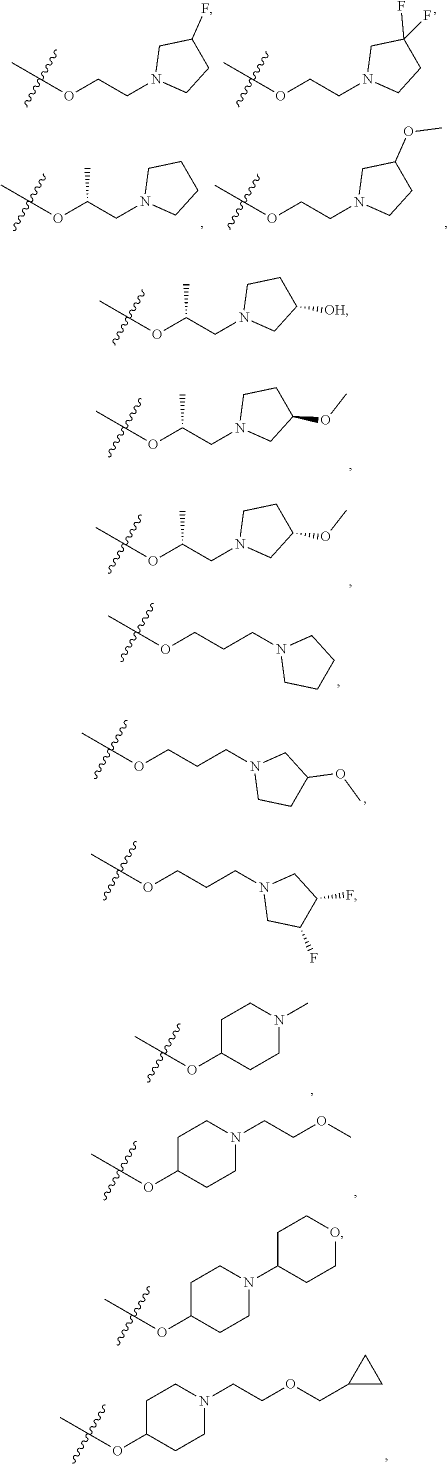

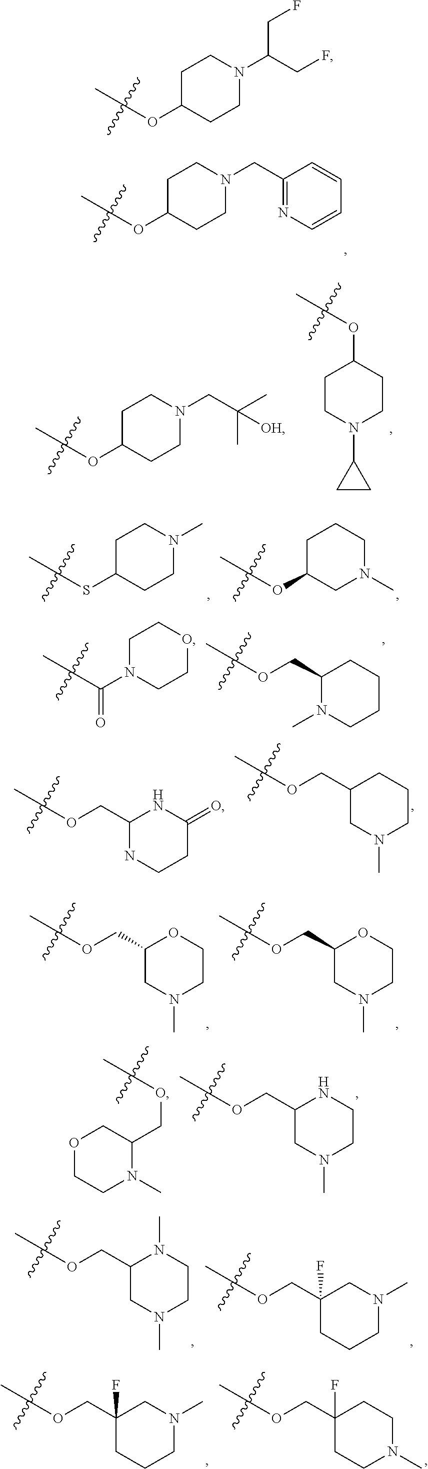

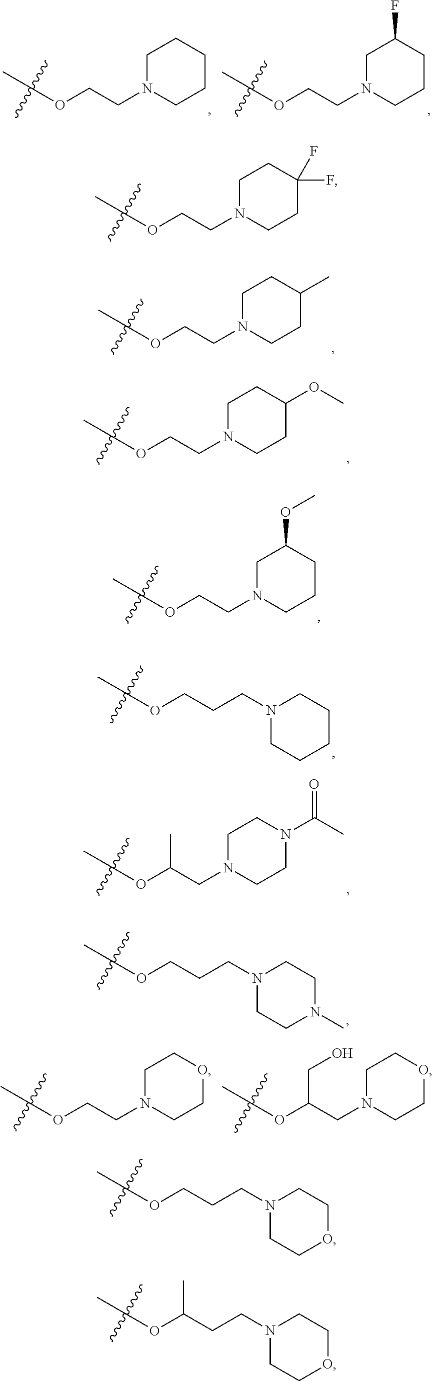

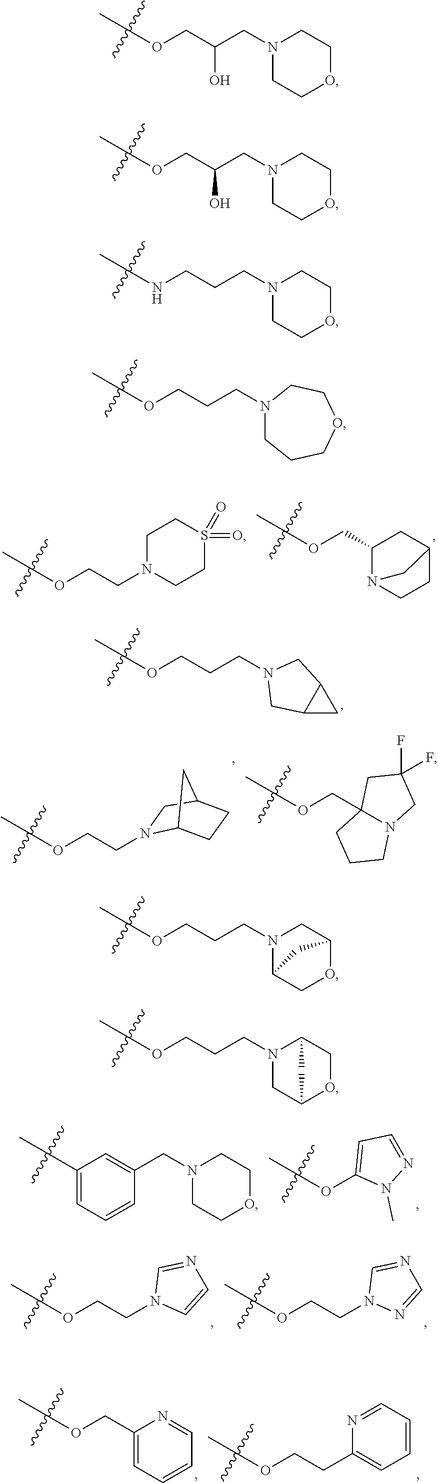

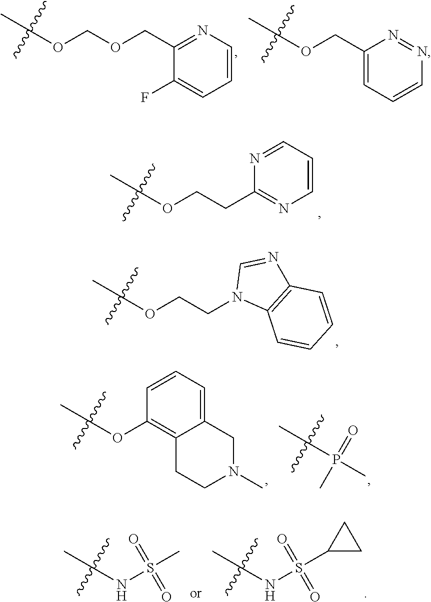

In some embodiments, each of L₄ at each occurrence is independently selected from absent, (CR₅R₆)ₘ, or NR₅;

Each of R₅ and R₆ in L₄ at each occurrence is independently selected from hydrogen, methyl, ethyl, propyl or isopropyl;

m in L₄ is selected from 1, 2 or 3.

In some embodiments, each of L₄ at each occurrence is independently selected from absent or NH.

In some embodiments, each of R₄ at each occurrence is independently selected from

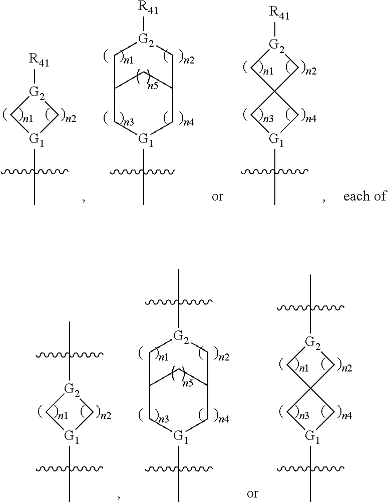

at each occurrence is independently optionally substituted by 1, 2, 3, 4, 5 or 6 R₄₂.

In some embodiments, each of G₁ and G₂ at each occurrence is independently selected from N or CR₅;

Each of R₅ in G₁ or G₂ at each occurrence is independently selected from hydrogen, methyl, ethyl, propyl or isopropyl.

In some embodiments, each of G₁ at each occurrence is independently selected from N or CH and each of G₂ at each occurrence is independently selected from N or CH.

In some embodiments, each of G₁ at each occurrence is independently selected from N or CH and each of G₂ at each occurrence is independently selected from N.

In some embodiments, each of n1, n2, n3, n4, n5 at each occurrence is independently selected from 0, 1, 2 or 3, provided that n1 and n2 is not 0 at the same time, n3 and n4 is not 0 at the same time.

In some embodiments, each of n1, n2, n3, n4, n5 at each occurrence is independently selected from 1 or 2, provided that n1 and n2 is not 0 at the same time, n3 and n4 is not 0 at the same time.

In some embodiments, each of R₄ at each occurrence is independently selected from

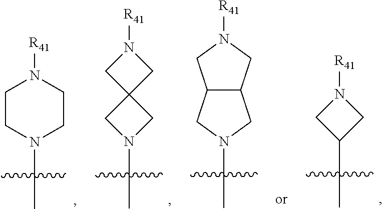

each of

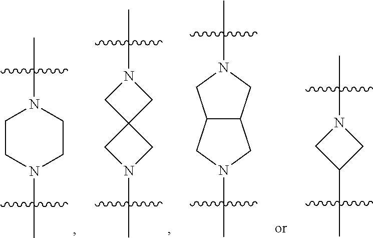

at each occurrence is independently optionally substituted by 1, 2, 3, 4, 5 or 6 R₄₂.

In some embodiments, each of R₄₁ at each occurrence is independently selected from

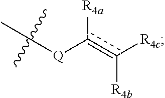

Each of Q at each occurrence is independently selected from —C(═O)—.

In some embodiments, each of R₄₁ at each occurrence is independently selected from

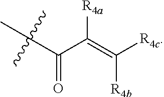

In some embodiments, each of R₄ₐ, R_{4b} and R_{4c} at each occurrence is independently selected from hydrogen, —F, —Cl, —Br, —C₁₋₃alkyl, —C₁₋₃alkylene-(halo)₁₋₃, heteroC₂₋₃alkyl, —CN, —OR₈, —C₁₋₃alkylene-OR₈, —NR₈R₉, —C₁₋₃alkylene-NR₈R₉, —NR₈—C₁₋₆alkylene-OR₈, —C(═O)R₈, —C(═O)OR₈, —OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(═O)₂NR₈R₉, —C₃₋₆carbocyclic, 3-6 membered heterocyclic or —C₁₋₃alkylene-(3-6 membered heterocyclic), each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

R_{4b} and R_{4c} together with the carbon which they both attach to form a C₃₋₆ carbocyclic ring or a 3-6 membered heterocyclic ring, or R₄ₐ and R_{4c} with the carbon they respectively attach to form a C₃-6 carbocyclic ring or a 3-6 membered heterocyclic ring; each of heterocyclic at each occurrence contains 1, 2 or 3 heteroatoms selected from N, O or S, and each of the carbocyclic or heterocyclic may be optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉when [symbol] is selected from ═;

Each of R₄ₐ is absent and one of R_{4b} and R_{4c} is absent, another of R_{4b} and R_{4c} is selected from hydrogen, —F, —Cl, —Br, oxo, —C₁₋₃alkyl, —C₁₋₃alkylene-(halo) heteroC₁₋₃alkyl, —CN, —OR₈, —C₁₋₃alkylene-OR₈, —NR₈R₉, —C₁₋₃alkylene-NR₈R₉, —C(═O)R₈, —C(═O)OR₈, —OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(═O)₂NR₈R₉ or —C₃₋₆carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉ when [symbol] is selected from ≡;

Each of R₈ and R₉ in R₄ₐ, R_{4b} or R_{4c} at each occurrence is independently selected from hydrogen or —C₁₋₃alkyl.

In some embodiments, each of R₄ₐ, R_{4b} and R_{4c} at each occurrence is independently selected from hydrogen, —F, —Cl, methyl, ethyl, propyl, isopropyl, -methylene-(halo)₁₋₃, -ethylene-(halo)₁₋₃, -propylene-(halo)₁₋₃, heteroethyl, heteropropyl, —CN, —OR₈, -methylene-OR₈, -ethylene-OR₈, -propylene-OR₈, —NR₈R₉, -methylene-NR₈R₉, -ethylene-NR₈R₉, -propylene-NR₈R₉, —NR₈-methylene-OR₈, —NR₈-ethylene-OR₈, —NR₈-propylene-OR₈, —C(═O)R₈, —C(═O)OR₈, —OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈, —S(═O)₂NR₈R₉, 3 membered carbocyclic, 4 membered carbocyclic, 5 membered carbocyclic, 6 membered carbocyclic, 3 membered heterocyclic, 4 membered heterocyclic, 5 membered heterocyclic, 6 membered heterocyclic, -methylene-(3-6 heterocyclic), -ethylene-(3-6 heterocyclic) or -propylene-(3-6 heterocyclic); each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

R_{4b} and R_{4c} together with the carbon which they both attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring, or R₄ₐ and R_{4c} with the carbon they respectively attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring; each of heterocyclic at each occurrence contains 1 or 2 heteroatoms selected from N or O, and each of carbocyclic or heterocyclic may be optionally substituted by 1, 2, 3, 4, 5 or 6 substituents selected from —F, —Cl, methyl, ethyl, propyl, isopropyl, methoxy, ethoxy, propoxy, isopropyl, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉ when [symbol] is selected from ≡;

Each of R₄ₐ is absent and one of R_{4b} and R_{4c} is absent, another of R_{4b} and R_{4c} is selected from hydrogen, —F, —Cl, oxo, methyl, ethyl, propyl, isopropyl, -methylene-(halo)₁₋₃, -ethylene-(halo)₁₋₃, -propylene-(halo)₁₋₃, heteroethyl, heteropropthyl, —CN, —OR₈, -methylene-OR₈, -ethylene-OR₈, -propylene-OR₈, —NR₈R₉, -methylene-NR₈R₉, -ethylene-NR₈R₉, -propylene-NR₈R₉, —C(═O)R₈, —C(═O)OR₈, —OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈, —S(═O)₂NR₈R₉, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉ when [symbol] is selected from ≡;

Each of R₈ and R₉ in R₄ₐ, R_{4b} or R_{4c} at each occurrence is independently selected from hydrogen, methyl, ethyl, propyl or isopropyl.

In some embodiments, each of R₄ₐ, R_{4b} and R_{4c} at each occurrence is independently selected from hydrogen, —F, —Cl, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂NHCH₃, —CH₂CH₂N(CH₃)₂, —CH₂CH₂CH₂N(CH₃)₂, —NHCH₂OH, —NHCH₂CH₂OH, —NHCH₂CH₂CH₂OH, —C(═O)CH₃, —COOH, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(═O)₂NH₂, —S(═O)₂NH(CH₃), —S(═O)₂N(CH₃)₂, 3 membered carbocyclic, 4 membered carbocyclic, 5 membered carbocyclic, 6 membered carbocyclic,

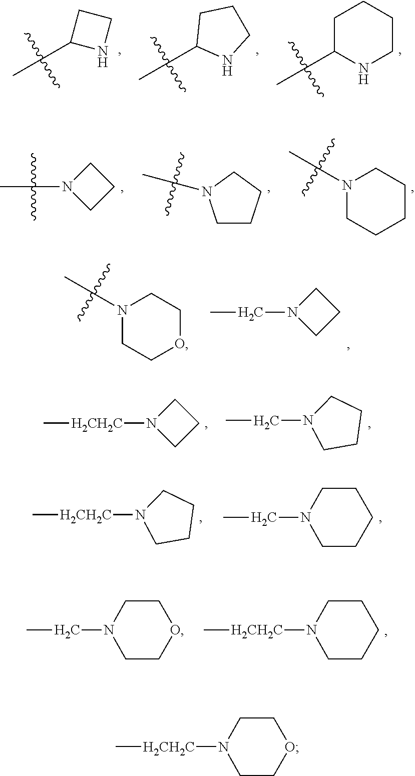

each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂; or

R_{4b} and R_{4c} with the carbon which they both attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic, or R₄ₐ and R_{4b} with the carbon they respectively attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring; each of heterocyclic at each occurrence contains 1 or 2 heteroatoms selected from N or O, and each of carbocyclic or heterocyclic may be optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂ when [symbol] is selected from ≡; or

Each of R₄ₐ is absent and one of R_{4b} and R_{4c} is absent, another of R_{4b} and R_{4c} is selected from hydrogen, —F, —Cl, oxo, methyl, ethyl, propyl, isopropyl, —CH₂F, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, —CN, —OH, —OCH₃, —OCH₂CH₃, —OCH₂CH₂CH₃, —OCH(CH₃)₂, —CH₂OH, —CH₂CH₂OH, —CH₂CH₂CH₂OH, —NH₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —C(═O)CH₃, —C(═O)OCH₃, —C(═O)OCH₂CH₃, —C(═O)OCH₂CH₂CH₃, —OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(═O)₂NH₂, —S(═O)₂NH(CH₃), —S(═O)₂N(CH₃)₂, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂ when [symbol] is selected from ≡.

In some embodiments, each of R₄ₐ, R_{4b} and R_{4c} at each occurrence is independently selected from hydrogen, —H, —F, —Cl, —CH₃, —CH₂F, —CF₃, —CH₂OH, —CH₂OCH₃, —CN, —N(CH₃)₂, —CH₂CH₂NH₂, —COOH, —NHCOCH₃,

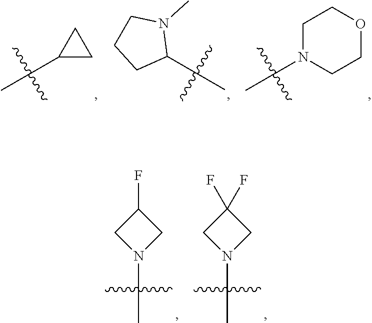

or R₄ₐ and R_{4c} with the carbon they respectively attach to form,

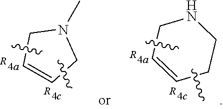

In some embodiments, each of R₄₁ at each occurrence is independently selected from

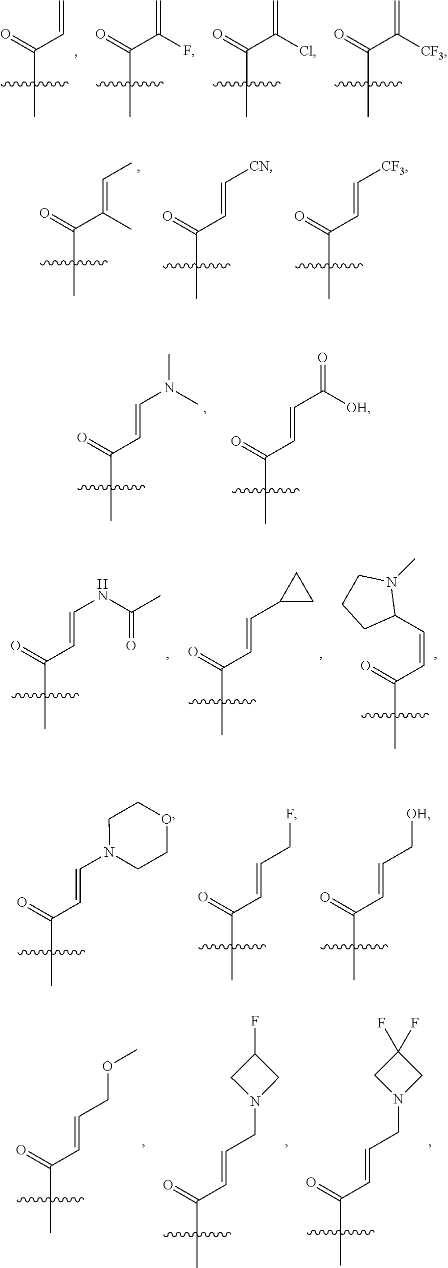

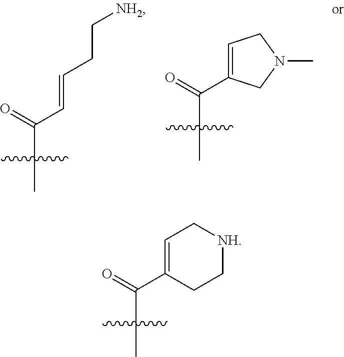

In some embodiments, each of R₄₂ is selected from —F, —Cl, —Br, oxo, —C₁₋₃alkyl, —C₁₋₃alkylene-(halo)₁₋₃, heteroC₂₋₃alkyl, —C₂₋₅alkenyl, —C₂₋₅alkynyl, —OR₈, —C₁₋₃alkylene-OR₈, —NR₈R₉, —C₁₋₃alkylene-NR₈R₉, —CN, —C₁₋₃alkylene-CN, —C(═O)R₈, —C₁₋₃alkylene-C(═O)R₈, —C(═O)OR₈, —C₁₋₃alkylene-C(═O)OR₈, —OC(═O)R₈, —C₁₋₃alkylene-OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(═O)₂NR₈R₉, —C₁₋₃alkylene-S(═O)₂NR₈R₉, —SO₂R₈, —C₁₋₃alkyleneSO₂R₈, —NR₈SO₂R₈, —C₁₋₃alkylene-NR₈SO₂R₈ or —C₃₋₆carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

Two R₄₂ together with the atom which they both or respectively attach to form a C₃₋₆ carbocyclic or 3-6 membered heterocyclic ring, each of the heterocyclic ring independently contains 1 or 2 heteroatoms selected from N or O, each of which optionally is substituted by 1, 2, 3, 4, 5 or 6 substituents selected from halogen, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₄₂ at each occurrence is independently selected from hydrogen or —C₁₋₃alkyl. In some embodiments, each of R₄₂ is selected from —F, —Cl, —Br, oxo, —C₁₋₃alkyl, —C₁₋₃alkylene-(halo)₁₋₃, heteroC₂₋₃alkyl, —C₂₋₅alkenyl, —C₂₋₅alkynyl, —OR₈, —C₁₋₃alkylene-OR₈, —NR₈R₉, —C₁₋₃alkylene-NR₈R₉, —CN, —C₁₋₃alkylene-CN, —C(═O)R₈, —C₁₋₃alkylene-C(═O)R₈, —C(═O)OR₈, —C₁₋₃alkylene-C(═O)OR₈, —OC(═O)R₈, —C₁₋₃alkylene-OC(═O)R₈, —C(═O)NR₈R₉, —C₁₋₃alkylene-C(═O)NR₈R₉, —NR₈C(═O)R₈, —C₁₋₃alkylene-NR₈C(═O)R₈, —S(═O)₂NR₈R₉, —C₁₋₃alkylene-S(═O)₂NR₈R₉, —SO₂R₈, —C₁₋₃alkyleneSO₂R₈, —NR₈SO₂R₈, —C₁₋₃alkylene-NR₈SO₂R₈, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

Two R₄₂ together with the atom which they both or respectively attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring, each of the heterocyclic ring independently contains 1 or 2 heteroatoms selected from N or O, each of which optionally is substituted by 1, 2, 3, 4, 5 or 6 substituents selected from —F, —Cl, —Br, —C₁₋₃alkyl, —C₁₋₃alkoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; Each of R₈ and R₉ in R₄₂ at each occurrence is independently selected from hydrogen, methyl, ethyl, propyl or isopropyl.

In some embodiments, each of R₄₂ is selected from —F, —Cl, oxo, methyl, ethyl, propyl, isopropyl, -methylene-(halo)₁₋₃, -ethylene-(halo)₁₋₃, -propylene-(halo)₁₋₃, heteroethyl, heteropropyl, ethenyl, propenyl, butenyl, pentenyl, ethynyl, propynyl, butynyl, pentynyl, —OR₈, -methylene-(OR₈)₁₋₃, -ethylene-(OR₈)₁₋₃, -propylene-(OR₈)₁₋₃, —NR₈R₉, -methylene-NR₈R₉, -ethylene-NR₈R₉, -propylene-NR₈R₉, —CN, -methylene-CN, -ethylene-CN, -propylene-CN, —C(═O)R₈, -methylene-C(═O)R₈, -ethylene-C(═O)R₈, -propylene-C(═O)R₈, —C(═O)OR₈, -methylene-C(═O)OR₈, -ethylene-C(═O)OR₈, -propylene-C(═O)OR₈, —OC(═O)R₈, -methylene-OC(═O)R₈, -ethylene-OC(═O)R₈, -propylene-OC(═O)R₈, —C(═O)NR₈R₉, -methylene-C(═O)NR₈R₉, -ethylene-C(═O)NR₈R₉, -propylene-C(═O)NR₈R₉, —NR₈C(═O)R₈, -methylene-NR₈C(═O)R₈, -ethylene-NR₈C(═O)R₈, -propylene-NR₈C(═O)R₈, —S(═O)₂NR₈R₉, -methylene-S(═O)₂NR₈R₉, -ethylene-S(═O)₂NR₈R₉, -propylene-S(═O)₂NR₈R₉, —SO₂CH₃, -methylene-SO₂CH₃, -ethylene-SO₂CH₃, -propylene-SO₂CH₃, —NHSO₂CH₃, —N(CH₃)SO₂CH₃, -methylene-NHSO₂CH₃, -ethylene-NHSO₂CH₃, -propylene-NHSO₂CH₃, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉; or

Two R₄₂ together with the atom which they both or respectively attach to form a 3 membered carbocyclic, 4 membered carbocyclic, 5 membered carbocyclic, 6 membered carbocyclic, 3 membered heterocyclic, 4 membered heterocyclic, 5 membered heterocyclic, 6 membered heterocyclic ring, each of the heterocyclic ring independently contains 1 or 2 heteroatoms selected from N or O, each of which optionally is substituted by 1, 2, 3, 4, 5 or 6 substituents selected from —F, —Cl, methyl, ethyl, propyl, isopropyl, methoxy, ethoxy, propoxy, isopropoxy, oxo, —OR₈, —NR₈R₉, —CN, —C(═O)R₈, —C(═O)OR₈, —OC(═O)R₈, —C(═O)NR₈R₉, —NR₈C(═O)R₈ or —S(═O)₂NR₈R₉;

Each of R₈ and R₉ in R₄₂ at each occurrence is independently selected from hydrogen, methyl, ethyl, propyl or isopropyl.

In some embodiments, each of R₄₂ is selected from —F, —Cl, oxo, methyl, ethyl, propyl, isopropyl, —CH₂F, —CH₂Cl, —CHF₂, —CF₃, —CH₂CH₂F, —CH₂CHF₂, —CH₂CF₃, —CH₂CH₂CH₂F, —CH₂CH₂CHF₂, —CH₂CH₂CF₃, —CH₂OCH₃, —CH₂CH₂OCH₃, —CH₂CH₂CH₂OCH₃, ethenyl, propenyl, butenyl, pentenyl, ethynyl, propynyl, butynyl, pentynyl, —OH, —OCH₃, —OCH₂CH₃, —OCH₂CH₂CH₃, —OCH(CH₃)₂, —CH₂OH, —CH₂CH₂OH, —CH₂CH₂CH₂OH, —C(OH)(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH₂N(CH₃)₂, —CH₂CH₂N(CH₃)₂, —CH₂CH₂CH₂N(CH₃)₂, —CN, —CH₂CN, —CH₂CH₂CN, —CH₂CH₂CN, —C(═O)CH₃, —CH₂C(═O)CH₃, —CH₂CH₂C(═O)CH₃, —CH₂CH₂CH₂C(═O)CH₃, —COOH, —CH₂COOH, —CH₂CH₂COOH, —C(═O)OCH₃, —CH₂C(═O)OCH₃, —CH₂CH₂C(═O)OCH₃, —CH₂CH₂CH₂C(═O)OCH₃, —C(═O)OCH₂CH₃, —C(═O)OCH₂CH₂CH₃, —OC(═O)CH₃, —CH₂OC(═O)CH₃, —CH₂CH₂OC(═O)CH₃, —CH₂CH₂CH₂OC(═O)CH₃, —C(═O)NH₂, —C(═O)NH(CH₃), —C(═O)N(CH₃)₂, —CH₂C(═O)N(CH₃)₂, —CH₂CH₂C(═O)N(CH₃)₂, —CH₂CH₂CH₂C(═O)N(CH₃)₂, —NHC(═O)CH₃, —N(CH₃)C(═O)CH₃, —CH₂NHC(═O)CH₃, —CH₂CH₂NHC(═O)CH₃, —CH₂CH₂CH₂NHC(═O)CH₃, —S(═O)₂NH₂, —S(═O)₂NH(CH₃), —S(═O)₂N(CH₃)₂, —CH₂S(═O)₂N(CH₃)₂, —CH₂CH₂S(═O)₂N(CH₃)₂ or —CH₂CH₂CH₂S(═O)₂N(CH₃)₂, —SO₂CH₃, —CH₂SO₂CH₃, —CH₂CH₂SO₂CH₃, —CH₂CH₂CH₂SO₂CH₃, —NHSO₂CH₃, —CH₂NHSO₂CH₃, —CH₂CH₂NHSO₂CH₃, —CH₂CH₂CH₂NHSO₂CH₃, 3 membered carbocyclic, 4 membered carbocyclic, 5 membered carbocyclic or 6 membered carbocyclic; each of which is independently optionally substituted by 1, 2, 3, 4, 5 or 6 substituents selected from —F, —Cl, methyl, ethyl, propyl, isopropyl, methoxy, ethoxy, propoxy, isopropoxy, oxo, —OH, —NH₂, —NHCH₃, —N(CH₃)₂, —CN, —C(═O)CH₃, —C(═O)OCH₃, —OC(═O)CH₃, —C(═O)NH₂, —C(═O)NH(CH₃), —C(═O)N(CH₃)₂, —NHC(═O)CH₃, —N(CH₃)C(═O)CH₃, —S(═O)₂NH₂, —S(═O)₂NH(CH₃) or —S(═O)₂N(CH₃)₂; or

Two R₄₂ together with the atom which they both or respectively attach to form a 3 membered carbocyclic, 4 membered carbocyclic, 5 membered carbocyclic, 6 membered carbocyclic, 3 membered heterocyclic, 4 membered heterocyclic, 5 membered heterocyclic or 6 membered heterocyclic ring, each of the heterocyclic ring independently contains 1 or 2 heteroatoms selected from N or O.

In some embodiments, each of R₄₂ is selected from —CH₃, —CH₂OH or —CH₂CN; or

Two R₄₂ together with the atom which they both or respectively attach to form

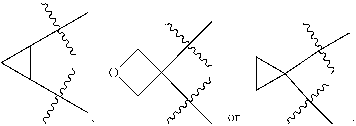 [SMILES: *C1CC1* ; *C1(*)COC1 ; *C1(*)CC1]

In some embodiments, each of R₄ is independently selected from:

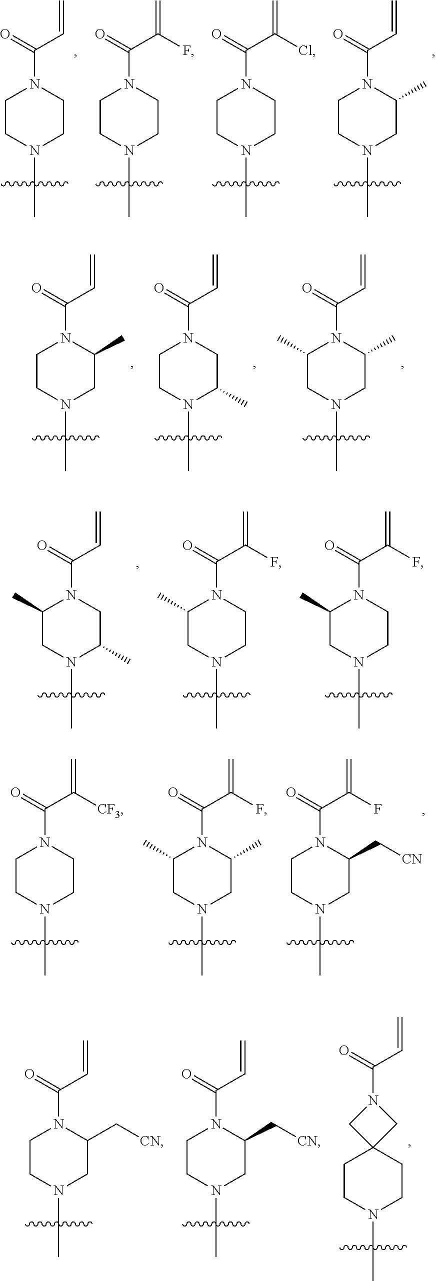

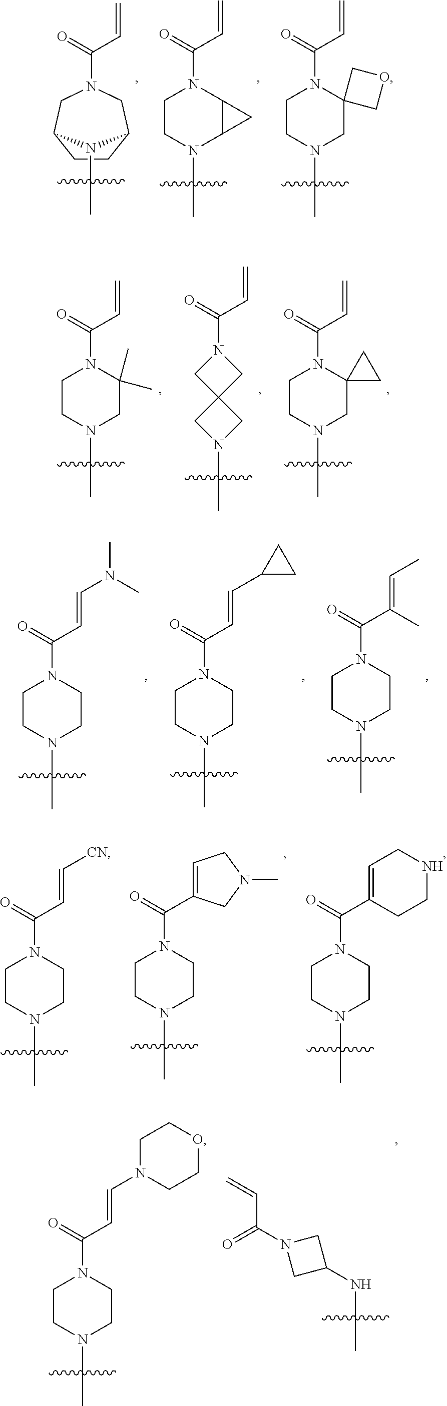

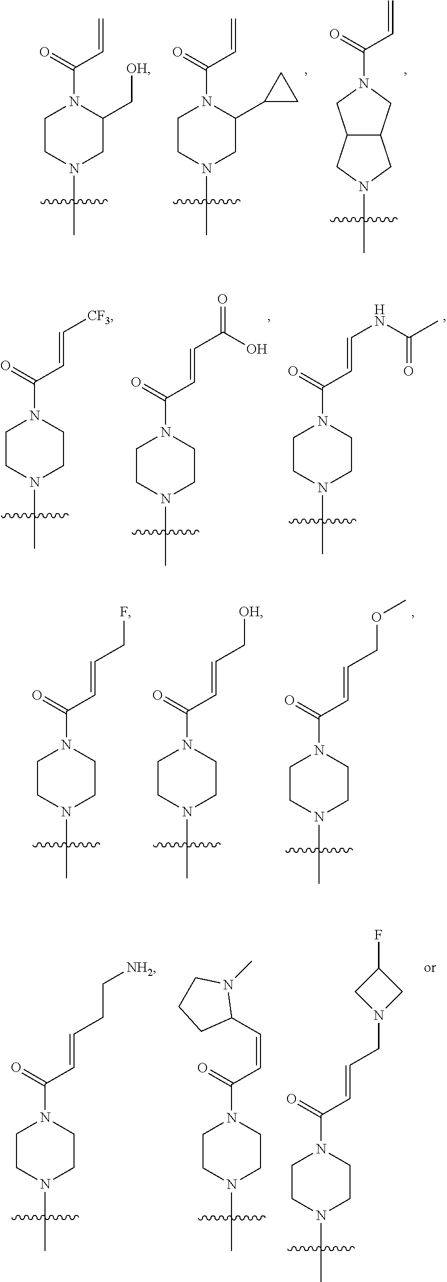

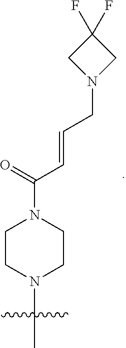 [SMILES: *N1CCN(C(=O)/C=C/CN2CC(F)(F)C2)CC1]

In some embodiments, the compound is selected from:

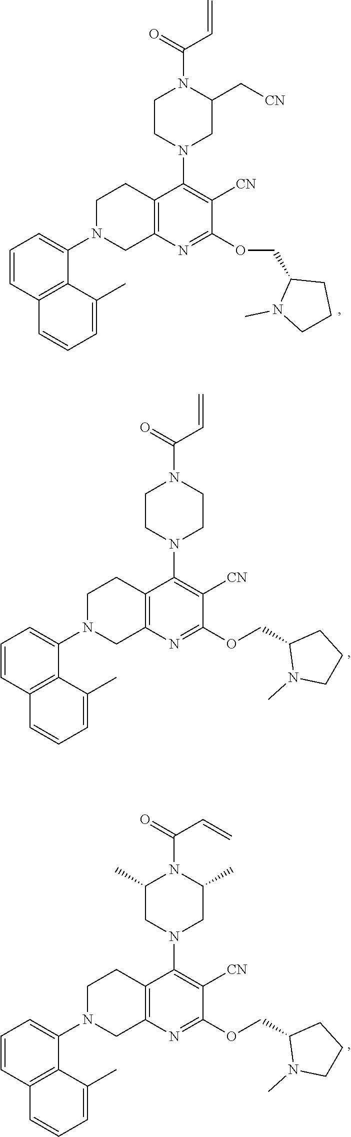 [SMILES: C=CC(=O)N1CCN(c2c(C#N)c(OC[C@@H]3CCCN3C)nc3c2CCN(c2cccc4cccc(C)c24)C3)CC1CC#N ; C=CC(=O)N1CCN(c2c(C#N)c(OC[C@@H]3CCCN3C)nc3c2CCN(c2cccc4cccc(C)c24)C3)CC1 ; C=CC(=O)N1[C@H](C)CN(c2c(C#N)c(OC[C@@H]3CCCN3C)nc3c2CCN(c2cccc4cccc(C)c24)C3)C[C@@H]1C]

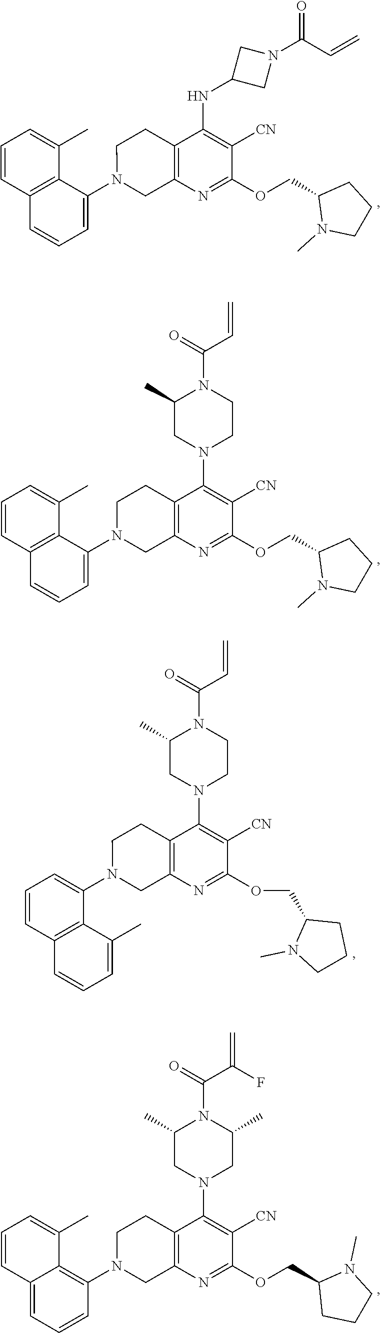 [SMILES: C=CC(=O)N1CC(Nc2c(C#N)c(OC[C@@H]3CCCN3C)nc3c2CCN(c2cccc4cccc(C)c24)C3)C1 ; C=CC(=O)N1CCN(c2c(C#N)c(OC[C@@H]3CCCN3C)nc3c2CCN(c2cccc4cccc(C)c24)C3)C[C@H]1C ; C=CC(=O)N1CCN(c2c(C#N)c(OC[C@@H]3CCCN3C)nc3c2CCN(c2cccc4cccc(C)c24)C3)C[C@@H]1C ; C=C(F)C(=O)N1[C@H](C)CN(c2c(C#N)c(OC[C@@H]3CCCN3C)nc3c2CCN(c2cccc4cccc(C)c24)C3)C[C@@H]1C]

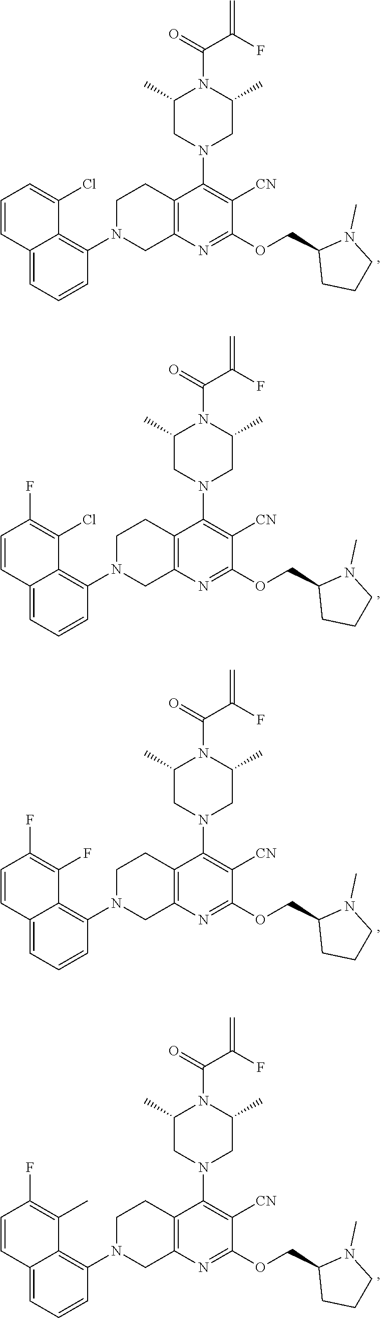 [SMILES: C=C(F)C(=O)N1[C@H](C)CN(c2c(C#N)c(OC[C@@H]3CCCN3C)nc3c2CCN(c2cccc4cccc(Cl)c24)C3)C[C@@H]1C ; C=C(F)C(=O)N1[C@H](C)CN(c2c(C#N)c(OC[C@@H]3CCCN3C)nc3c2CCN(c2cccc4ccc(F)c(Cl)c24)C3)C[C@@H]1C ; C=C(F)C(=O)N1[C@H](C)CN(c2c(C#N)c(OC[C@@H]3CCCN3C)nc3c2CCN(c2cccc4ccc(F)c(F)c24)C3)C[C@@H]1C ; C=C(F)C(=O)N1[C@H](C)CN(c2c(C#N)c(OC[C@@H]3CCCN3C)nc3c2CCN(c2cccc4ccc(F)c(C)c24)C3)C[C@@H]1C]

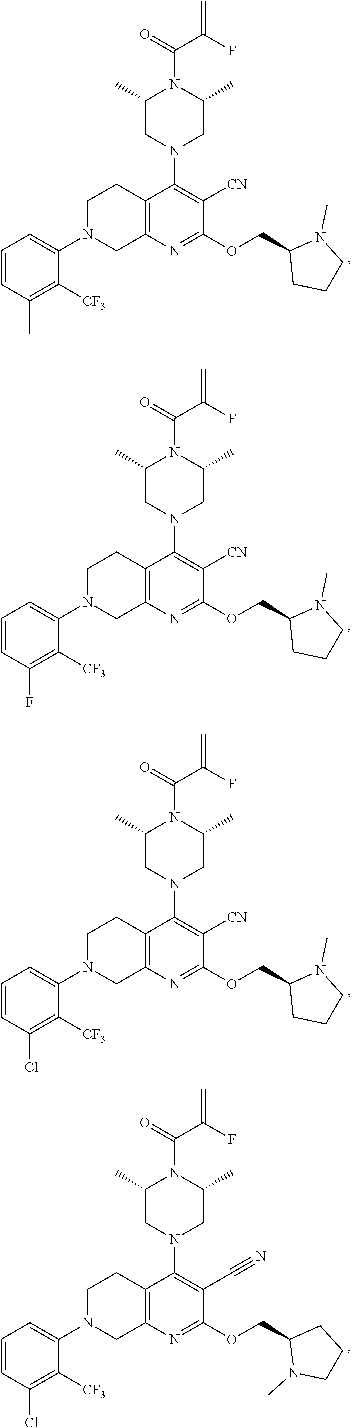 [SMILES: C=C(F)C(=O)N1[C@H](C)CN(c2c(C#N)c(OC[C@@H]3CCCN3C)nc3c2CCN(c2cccc(C)c2C(F)(F)F)C3)C[C@@H]1C ; C=C(F)C(=O)N1[C@H](C)CN(c2c(C#N)c(OC[C@@H]3CCCN3C)nc3c2CCN(c2cccc(F)c2C(F)(F)F)C3)C[C@@H]1C ; C=C(F)C(=O)N1[C@H](C)CN(c2c(C#N)c(OC[C@@H]3CCCN3C)nc3c2CCN(c2cccc(Cl)c2C(F)(F)F)C3)C[C@@H]1C ; C=C(F)C(=O)N1[C@H](C)CN(c2c(C#N)c(OC[C@H]3CCCN3C)nc3c2CCN(c2cccc(Cl)c2C(F)(F)F)C3)C[C@@H]1C]

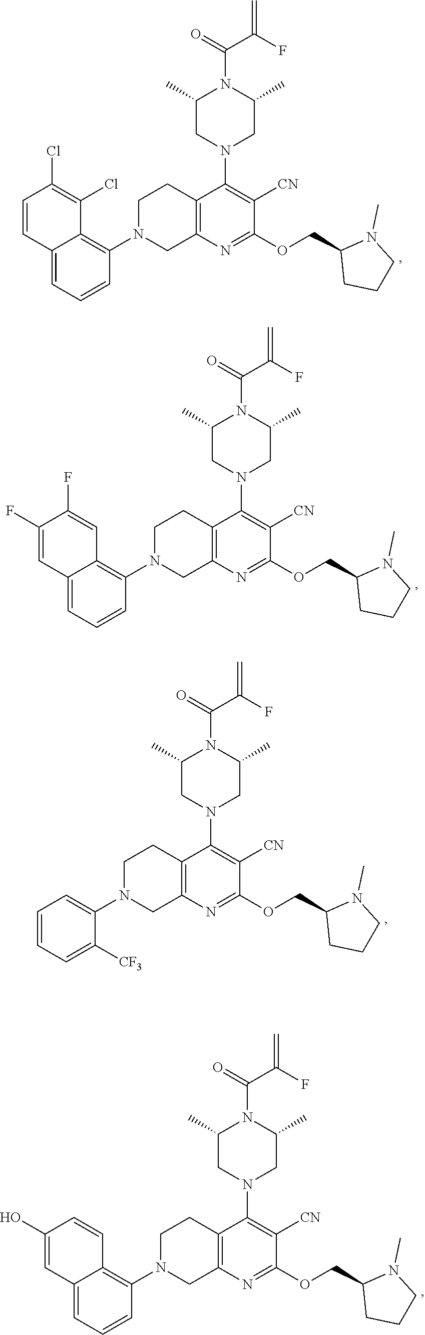 [SMILES: C=C(F)C(=O)N1[C@H](C)CN(c2c(C#N)c(OC[C@@H]3CCCN3C)nc3c2CCN(c2cccc4ccc(Cl)c(Cl)c24)C3)C[C@@H]1C ; C=C(F)C(=O)N1[C@H](C)CN(c2c(C#N)c(OC[C@@H]3CCCN3C)nc3c2CCN(c2cccc4cc(F)c(F)cc24)C3)C[C@@H]1C ; C=C(F)C(=O)N1[C@H](C)CN(c2c(C#N)c(OC[C@@H]3CCCN3C)nc3c2CCN(c2ccccc2C(F)(F)F)C3)C[C@@H]1C ; C=C(F)C(=O)N1[C@H](C)CN(c2c(C#N)c(OC[C@@H]3CCCN3C)nc3c2CCN(c2cccc4cc(O)ccc24)C3)C[C@@H]1C]

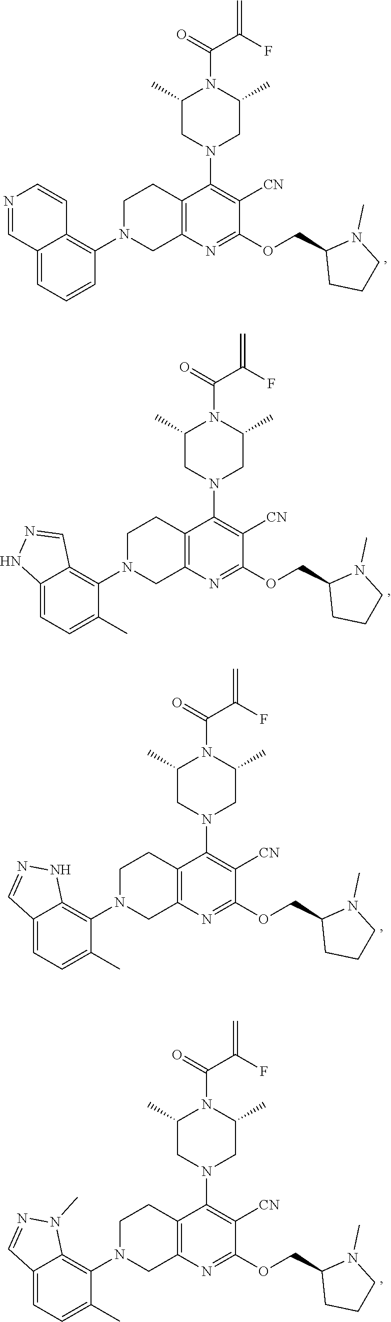 [SMILES: C=C(F)C(=O)N1[C@H](C)CN(c2c(C#N)c(OC[C@@H]3CCCN3C)nc3c2CCN(c2cccc4cnccc24)C3)C[C@@H]1C ; C=C(F)C(=O)N1[C@H](C)CN(c2c(C#N)c(OC[C@@H]3CCCN3C)nc3c2CCN(c2c(C)ccc4[nH]ncc24)C3)C[C@@H]1C ; C=C(F)C(=O)N1[C@H](C)CN(c2c(C#N)c(OC[C@@H]3CCCN3C)nc3c2CCN(c2c(C)ccc4cn[nH]c24)C3)C[C@@H]1C ; C=C(F)C(=O)N1[C@H](C)CN(c2c(C#N)c(OC[C@@H]3CCCN3C)nc3c2CCN(c2c(C)ccc4cnn(C)c24)C3)C[C@@H]1C]

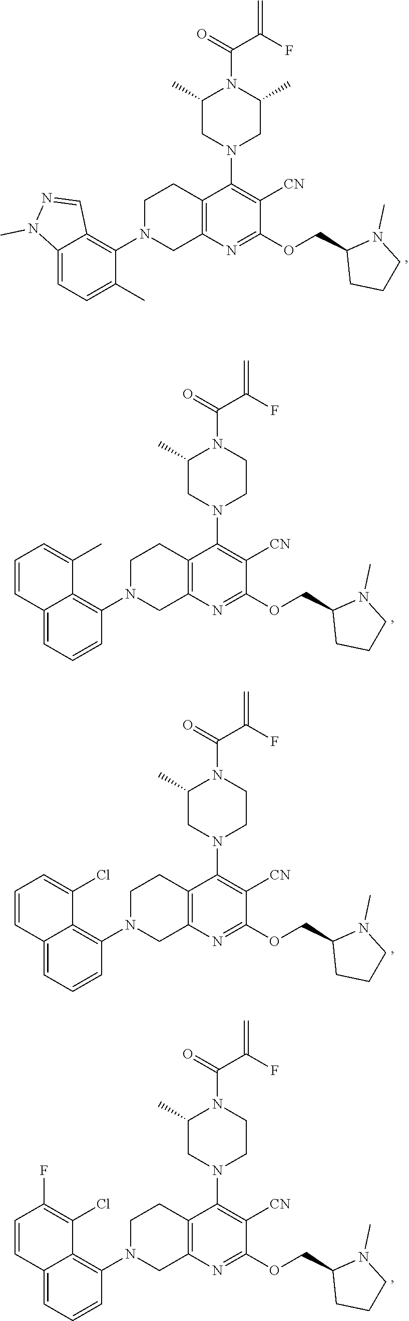 [SMILES: C=C(F)C(=O)N1[C@H](C)CN(c2c(C#N)c(OC[C@@H]3CCCN3C)nc3c2CCN(c2c(C)ccc4c2cnn4C)C3)C[C@@H]1C ; C=C(F)C(=O)N1CCN(c2c(C#N)c(OC[C@@H]3CCCN3C)nc3c2CCN(c2cccc4cccc(C)c24)C3)C[C@@H]1C ; C=C(F)C(=O)N1CCN(c2c(C#N)c(OC[C@@H]3CCCN3C)nc3c2CCN(c2cccc4cccc(Cl)c24)C3)C[C@@H]1C ; C=C(F)C(=O)N1CCN(c2c(C#N)c(OC[C@@H]3CCCN3C)nc3c2CCN(c2cccc4ccc(F)c(Cl)c24)C3)C[C@@H]1C]

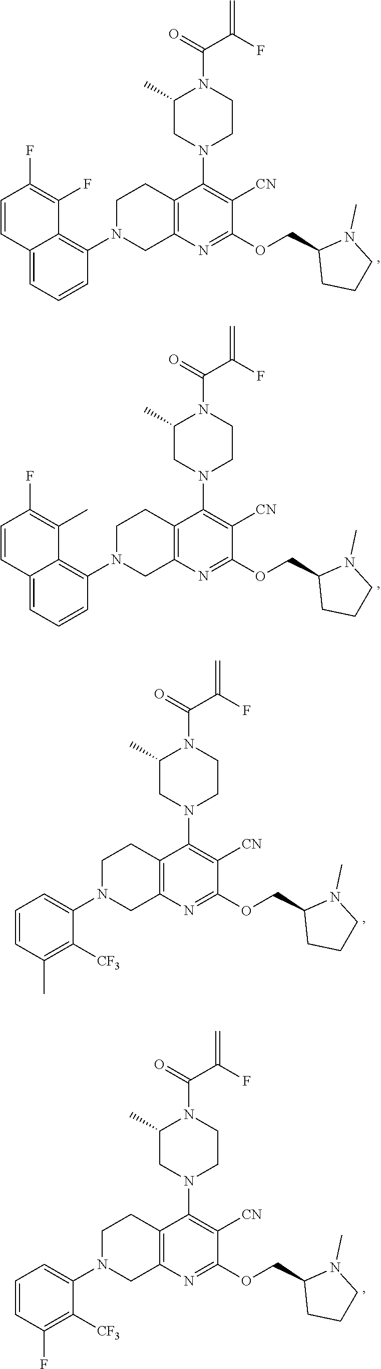 [SMILES: C=C(F)C(=O)N1CCN(c2c(C#N)c(OC[C@@H]3CCCN3C)nc3c2CCN(c2cccc4ccc(F)c(F)c24)C3)C[C@@H]1C ; C=C(F)C(=O)N1CCN(c2c(C#N)c(OC[C@@H]3CCCN3C)nc3c2CCN(c2cccc4ccc(F)c(C)c24)C3)C[C@@H]1C ; C=C(F)C(=O)N1CCN(c2c(C#N)c(OC[C@@H]3CCCN3C)nc3c2CCN(c2cccc(C)c2C(F)(F)F)C3)C[C@@H]1C ; C=C(F)C(=O)N1CCN(c2c(C#N)c(OC[C@@H]3CCCN3C)nc3c2CCN(c2cccc(F)c2C(F)(F)F)C3)C[C@@H]1C]

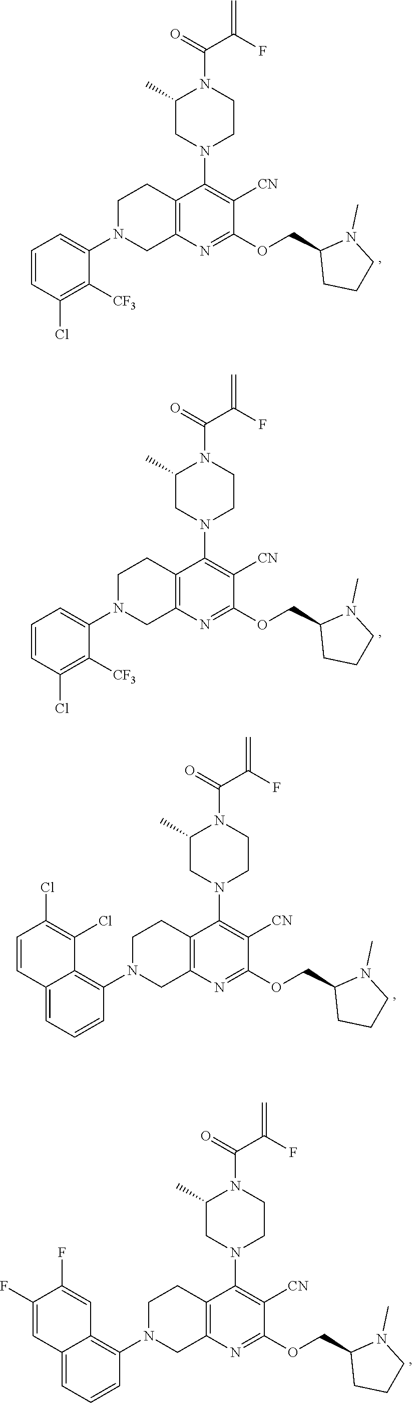 [SMILES: C=C(F)C(=O)N1CCN(c2c(C#N)c(OC[C@@H]3CCCN3C)nc3c2CCN(c2cccc(Cl)c2C(F)(F)F)C3)C[C@@H]1C ; C=C(F)C(=O)N1CCN(c2c(C#N)c(OC[C@@H]3CCCN3C)nc3c2CCN(c2cccc(Cl)c2C(F)(F)F)C3)C[C@@H]1C ; C=C(F)C(=O)N1CCN(c2c(C#N)c(OC[C@@H]3CCCN3C)nc3c2CCN(c2cccc4ccc(Cl)c(Cl)c24)C3)C[C@@H]1C ; C=C(F)C(=O)N1CCN(c2c(C#N)c(OC[C@@H]3CCCN3C)nc3c2CCN(c2cccc4cc(F)c(F)cc24)C3)C[C@@H]1C]

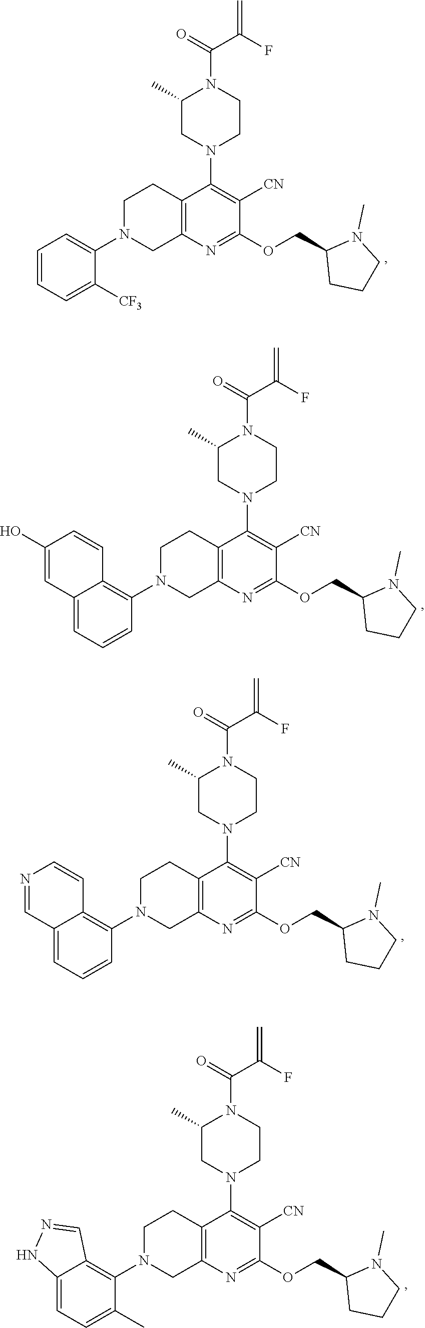 [SMILES: C=C(F)C(=O)N1CCN(c2c(C#N)c(OC[C@@H]3CCCN3C)nc3c2CCN(c2ccccc2C(F)(F)F)C3)C[C@@H]1C ; C=C(F)C(=O)N1CCN(c2c(C#N)c(OC[C@@H]3CCCN3C)nc3c2CCN(c2cccc4cc(O)ccc24)C3)C[C@@H]1C ; C=C(F)C(=O)N1CCN(c2c(C#N)c(OC[C@@H]3CCCN3C)nc3c2CCN(c2cccc4cnccc24)C3)C[C@@H]1C ; C=C(F)C(=O)N1CCN(c2c(C#N)c(OC[C@@H]3CCCN3C)nc3c2CCN(c2c(C)ccc4[nH]ncc24)C3)C[C@@H]1C]

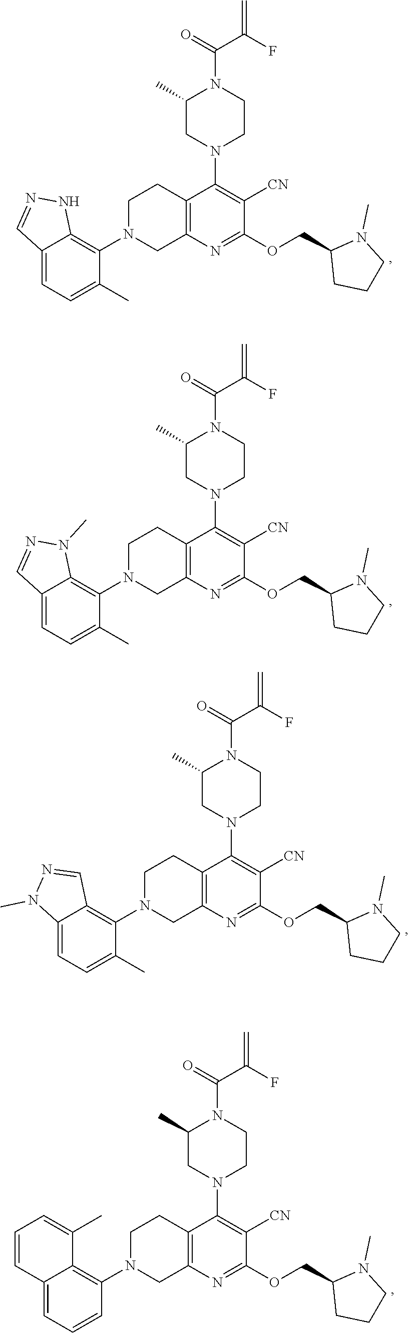 [SMILES: C=C(F)C(=O)N1CCN(c2c(C#N)c(OC[C@@H]3CCCN3C)nc3c2CCN(c2c(C)ccc4cn[nH]c24)C3)C[C@@H]1C ; C=C(F)C(=O)N1CCN(c2c(C#N)c(OC[C@@H]3CCCN3C)nc3c2CCN(c2c(C)ccc4cnn(C)c24)C3)C[C@@H]1C ; C=C(F)C(=O)N1CCN(c2c(C#N)c(OC[C@@H]3CCCN3C)nc3c2CCN(c2c(C)ccc4c2cnn4C)C3)C[C@@H]1C ; C=C(F)C(=O)N1CCN(c2c(C#N)c(OC[C@@H]3CCCN3C)nc3c2CCN(c2cccc4cccc(C)c24)C3)C[C@H]1C]

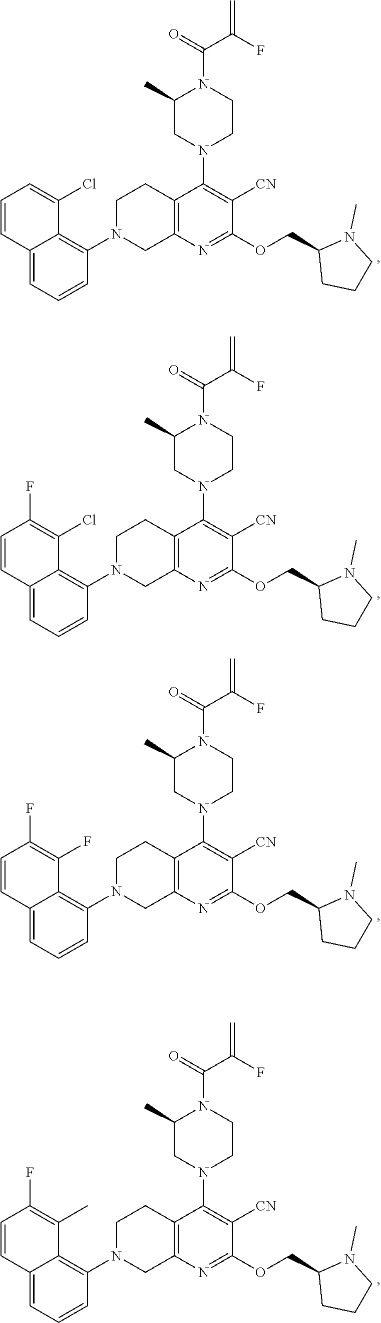 [SMILES: C=C(F)C(=O)N1CCN(c2c(C#N)c(OC[C@@H]3CCCN3C)nc3c2CCN(c2cccc4cccc(Cl)c24)C3)C[C@H]1C ; C=C(F)C(=O)N1CCN(c2c(C#N)c(OC[C@@H]3CCCN3C)nc3c2CCN(c2cccc4ccc(F)c(Cl)c24)C3)C[C@H]1C ; C=C(F)C(=O)N1CCN(c2c(C#N)c(OC[C@@H]3CCCN3C)nc3c2CCN(c2cccc4ccc(F)c(F)c24)C3)C[C@H]1C ; C=C(F)C(=O)N1CCN(c2c(C#N)c(OC[C@@H]3CCCN3C)nc3c2CCN(c2cccc4ccc(F)c(C)c24)C3)C[C@H]1C]

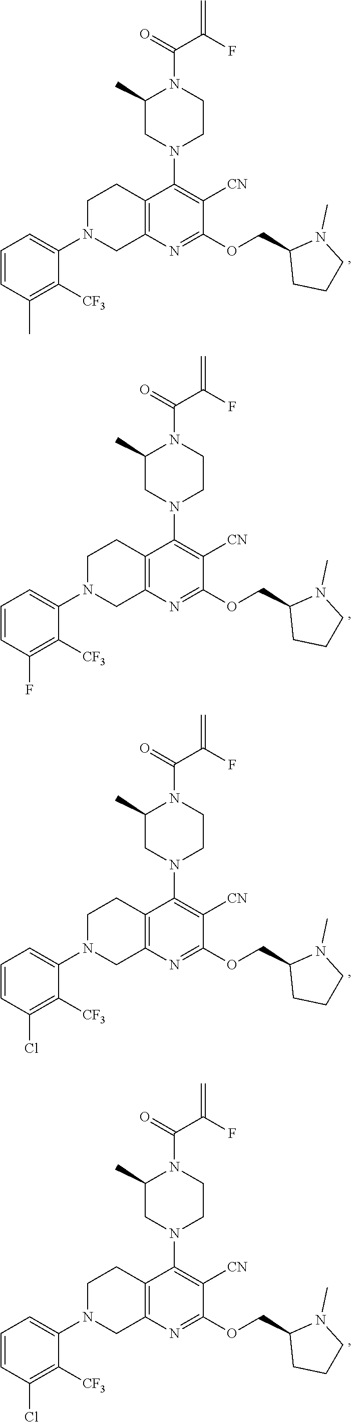 [SMILES: C=C(F)C(=O)N1CCN(c2c(C#N)c(OC[C@@H]3CCCN3C)nc3c2CCN(c2cccc(C)c2C(F)(F)F)C3)C[C@H]1C ; C=C(F)C(=O)N1CCN(c2c(C#N)c(OC[C@@H]3CCCN3C)nc3c2CCN(c2cccc(F)c2C(F)(F)F)C3)C[C@H]1C ; C=C(F)C(=O)N1CCN(c2c(C#N)c(OC[C@@H]3CCCN3C)nc3c2CCN(c2cccc(Cl)c2C(F)(F)F)C3)C[C@H]1C ; C=C(F)C(=O)N1CCN(c2c(C#N)c(OC[C@@H]3CCCN3C)nc3c2CCN(c2cccc(Cl)c2C(F)(F)F)C3)C[C@H]1C]

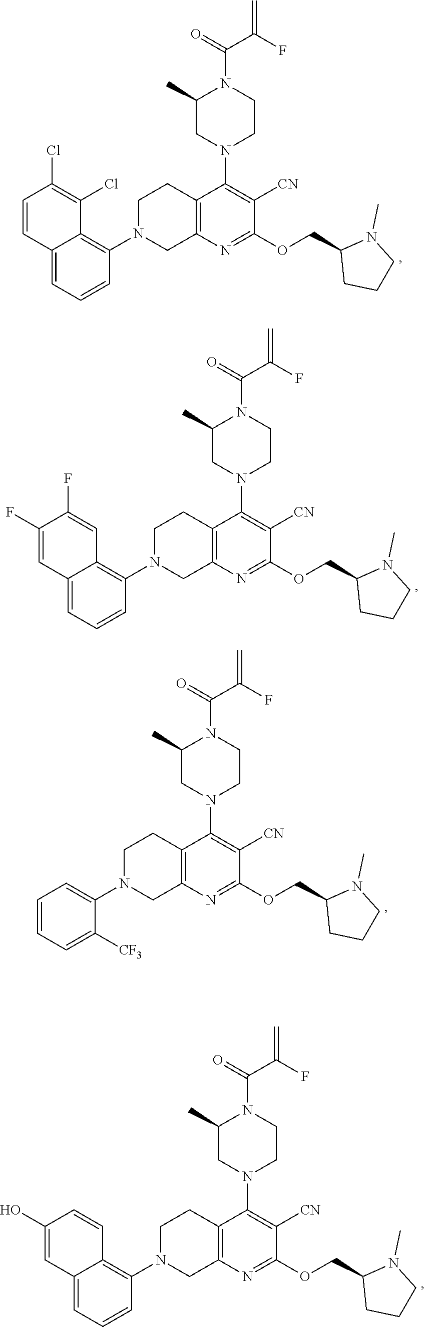 [SMILES: C=C(F)C(=O)N1CCN(c2c(C#N)c(OC[C@@H]3CCCN3C)nc3c2CCN(c2cccc4ccc(Cl)c(Cl)c24)C3)C[C@H]1C ; C=C(F)C(=O)N1CCN(c2c(C#N)c(OC[C@@H]3CCCN3C)nc3c2CCN(c2cccc4cc(F)c(F)cc24)C3)C[C@H]1C ; C=C(F)C(=O)N1CCN(c2c(C#N)c(OC[C@@H]3CCCN3C)nc3c2CCN(c2ccccc2C(F)(F)F)C3)C[C@H]1C ; C=C(F)C(=O)N1CCN(c2c(C#N)c(OC[C@@H]3CCCN3C)nc3c2CCN(c2cccc4cc(O)ccc24)C3)C[C@H]1C]

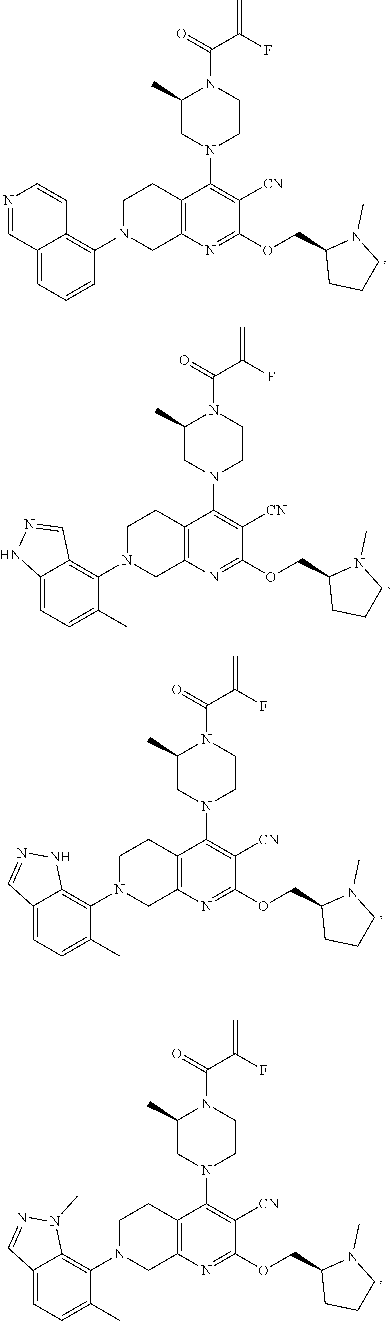 [SMILES: C=C(F)C(=O)N1CCN(c2c(C#N)c(OC[C@@H]3CCCN3C)nc3c2CCN(c2cccc4cnccc24)C3)C[C@H]1C ; C=C(F)C(=O)N1CCN(c2c(C#N)c(OC[C@@H]3CCCN3C)nc3c2CCN(c2c(C)ccc4[nH]ncc24)C3)C[C@H]1C ; C=C(F)C(=O)N1CCN(c2c(C#N)c(OC[C@@H]3CCCN3C)nc3c2CCN(c2c(C)ccc4cn[nH]c24)C3)C[C@H]1C ; C=C(F)C(=O)N1CCN(c2c(C#N)c(OC[C@@H]3CCCN3C)nc3c2CCN(c2c(C)ccc4cnn(C)c24)C3)C[C@H]1C]

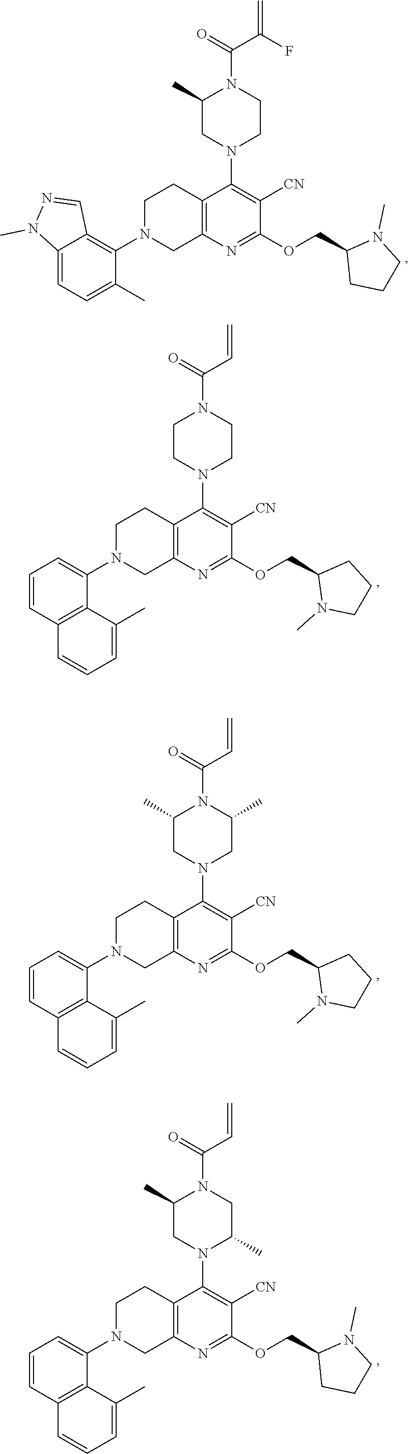 [SMILES: C=C(F)C(=O)N1CCN(c2c(C#N)c(OC[C@@H]3CCCN3C)nc3c2CCN(c2c(C)ccc4c2cnn4C)C3)C[C@H]1C ; C=CC(=O)N1CCN(c2c(C#N)c(OC[C@H]3CCCN3C)nc3c2CCN(c2cccc4cccc(C)c24)C3)CC1 ; C=CC(=O)N1[C@H](C)CN(c2c(C#N)c(OC[C@H]3CCCN3C)nc3c2CCN(c2cccc4cccc(C)c24)C3)C[C@@H]1C ; C=CC(=O)N1C[C@H](C)N(c2c(C#N)c(OC[C@@H]3CCCN3C)nc3c2CCN(c2cccc4cccc(C)c24)C3)C[C@H]1C]

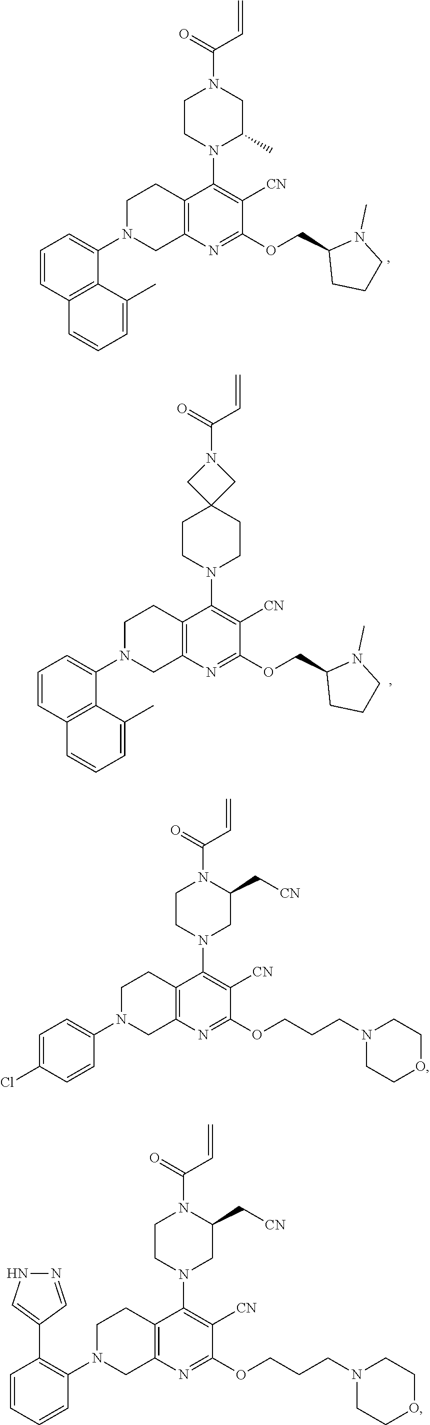 [SMILES: C=CC(=O)N1CCN(c2c(C#N)c(OC[C@@H]3CCCN3C)nc3c2CCN(c2cccc4cccc(C)c24)C3)[C@@H](C)C1 ; C=CC(=O)N1CC2(CCN(c3c(C#N)c(OC[C@@H]4CCCN4C)nc4c3CCN(c3cccc5cccc(C)c35)C4)CC2)C1 ; C=CC(=O)N1CCN(c2c(C#N)c(OCCCN3CCOCC3)nc3c2CCN(c2ccc(Cl)cc2)C3)C[C@@H]1CC#N ; C=CC(=O)N1CCN(c2c(C#N)c(OCCCN3CCOCC3)nc3c2CCN(c2ccccc2-c2cn[nH]c2)C3)C[C@@H]1CC#N]

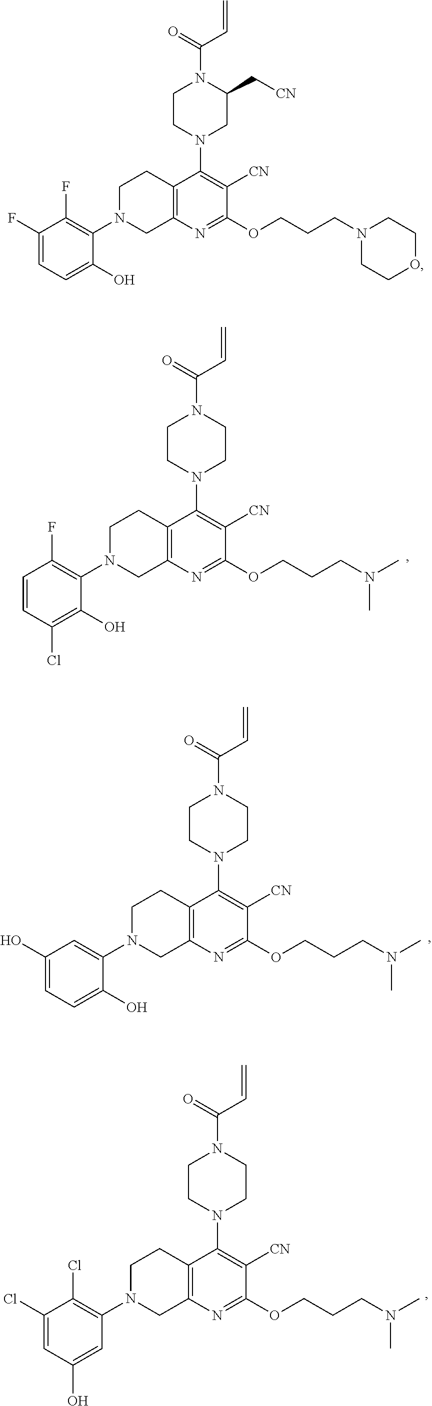 [SMILES: C=CC(=O)N1CCN(c2c(C#N)c(OCCCN3CCOCC3)nc3c2CCN(c2c(O)ccc(F)c2F)C3)C[C@@H]1CC#N ; C=CC(=O)N1CCN(c2c(C#N)c(OCCCN(C)C)nc3c2CCN(c2c(F)ccc(Cl)c2O)C3)CC1 ; C=CC(=O)N1CCN(c2c(C#N)c(OCCCN(C)C)nc3c2CCN(c2cc(O)ccc2O)C3)CC1 ; C=CC(=O)N1CCN(c2c(C#N)c(OCCCN(C)C)nc3c2CCN(c2cc(O)cc(Cl)c2Cl)C3)CC1]

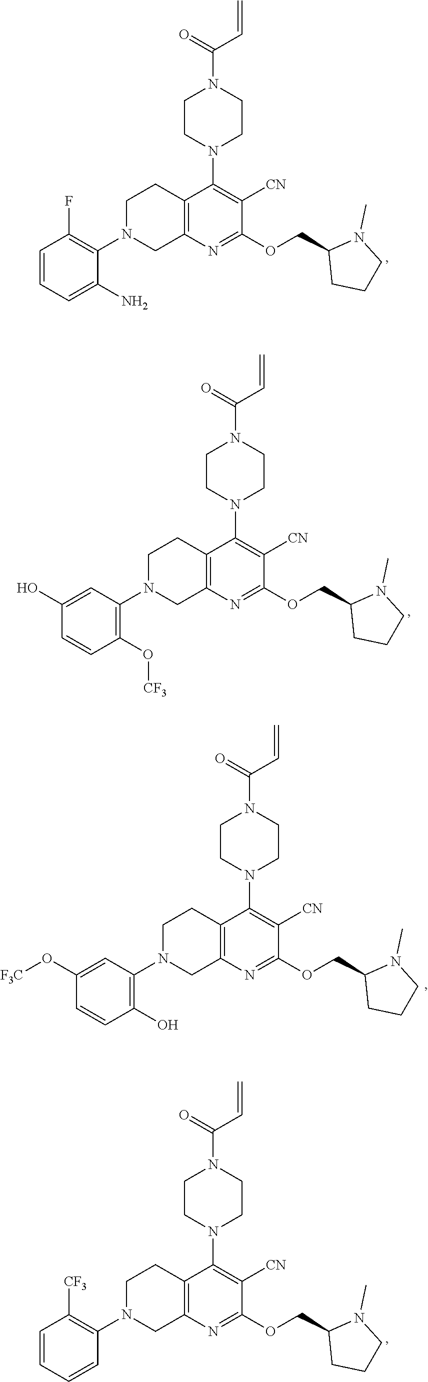 [SMILES: C=CC(=O)N1CCN(c2c(C#N)c(OC[C@@H]3CCCN3C)nc3c2CCN(c2c(N)cccc2F)C3)CC1 ; C=CC(=O)N1CCN(c2c(C#N)c(OC[C@@H]3CCCN3C)nc3c2CCN(c2cc(O)ccc2OC(F)(F)F)C3)CC1 ; C=CC(=O)N1CCN(c2c(C#N)c(OC[C@@H]3CCCN3C)nc3c2CCN(c2cc(OC(F)(F)F)ccc2O)C3)CC1 ; C=CC(=O)N1CCN(c2c(C#N)c(OC[C@@H]3CCCN3C)nc3c2CCN(c2ccccc2C(F)(F)F)C3)CC1]

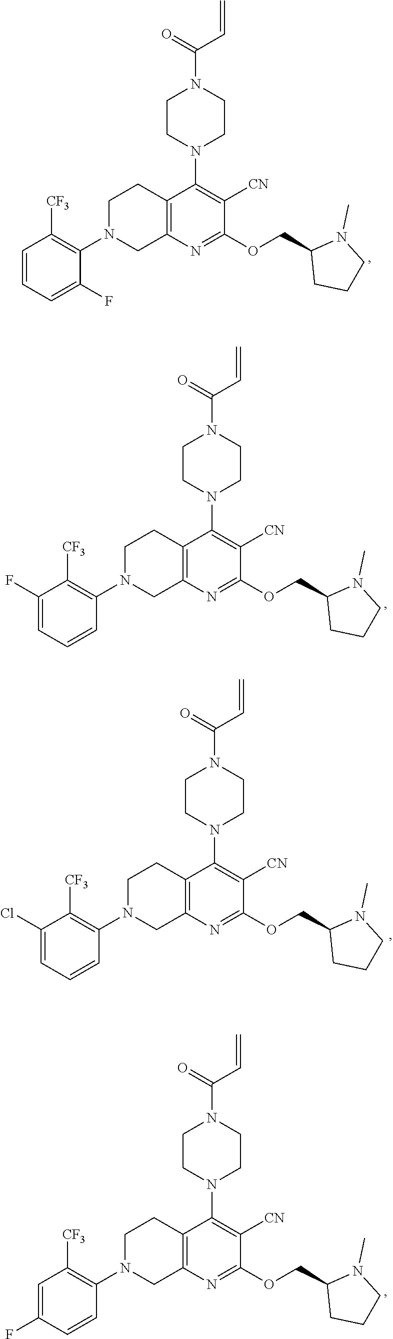 [SMILES: C=CC(=O)N1CCN(c2c(C#N)c(OC[C@@H]3CCCN3C)nc3c2CCN(c2c(F)cccc2C(F)(F)F)C3)CC1 ; C=CC(=O)N1CCN(c2c(C#N)c(OC[C@@H]3CCCN3C)nc3c2CCN(c2cccc(F)c2C(F)(F)F)C3)CC1 ; C=CC(=O)N1CCN(c2c(C#N)c(OC[C@@H]3CCCN3C)nc3c2CCN(c2cccc(Cl)c2C(F)(F)F)C3)CC1 ; C=CC(=O)N1CCN(c2c(C#N)c(OC[C@@H]3CCCN3C)nc3c2CCN(c2ccc(F)cc2C(F)(F)F)C3)CC1]

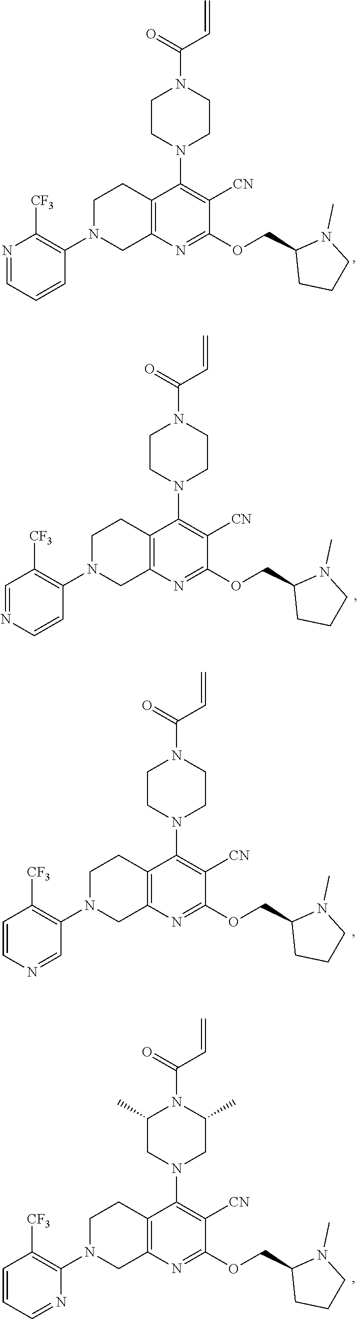 [SMILES: C=CC(=O)N1CCN(c2c(C#N)c(OC[C@@H]3CCCN3C)nc3c2CCN(c2cccnc2C(F)(F)F)C3)CC1 ; C=CC(=O)N1CCN(c2c(C#N)c(OC[C@@H]3CCCN3C)nc3c2CCN(c2ccncc2C(F)(F)F)C3)CC1 ; C=CC(=O)N1CCN(c2c(C#N)c(OC[C@@H]3CCCN3C)nc3c2CCN(c2cnccc2C(F)(F)F)C3)CC1 ; C=CC(=O)N1[C@H](C)CN(c2c(C#N)c(OC[C@@H]3CCCN3C)nc3c2CCN(c2ncccc2C(F)(F)F)C3)C[C@@H]1C]

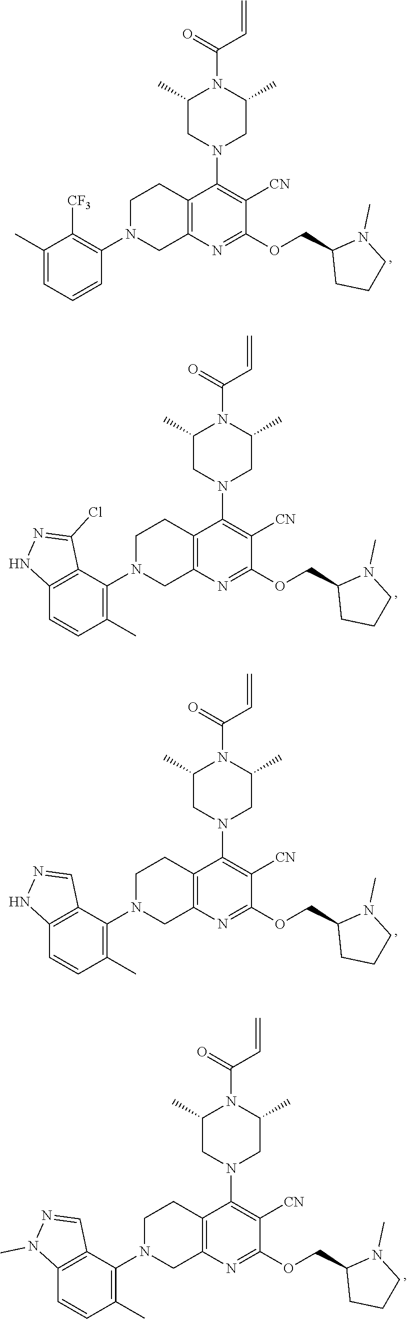 [SMILES: C=CC(=O)N1[C@H](C)CN(c2c(C#N)c(OC[C@@H]3CCCN3C)nc3c2CCN(c2cccc(C)c2C(F)(F)F)C3)C[C@@H]1C ; C=CC(=O)N1[C@H](C)CN(c2c(C#N)c(OC[C@@H]3CCCN3C)nc3c2CCN(c2c(C)ccc4[nH]nc(Cl)c24)C3)C[C@@H]1C ; C=CC(=O)N1[C@H](C)CN(c2c(C#N)c(OC[C@@H]3CCCN3C)nc3c2CCN(c2c(C)ccc4[nH]ncc24)C3)C[C@@H]1C ; C=CC(=O)N1[C@H](C)CN(c2c(C#N)c(OC[C@@H]3CCCN3C)nc3c2CCN(c2c(C)ccc4c2cnn4C)C3)C[C@@H]1C]

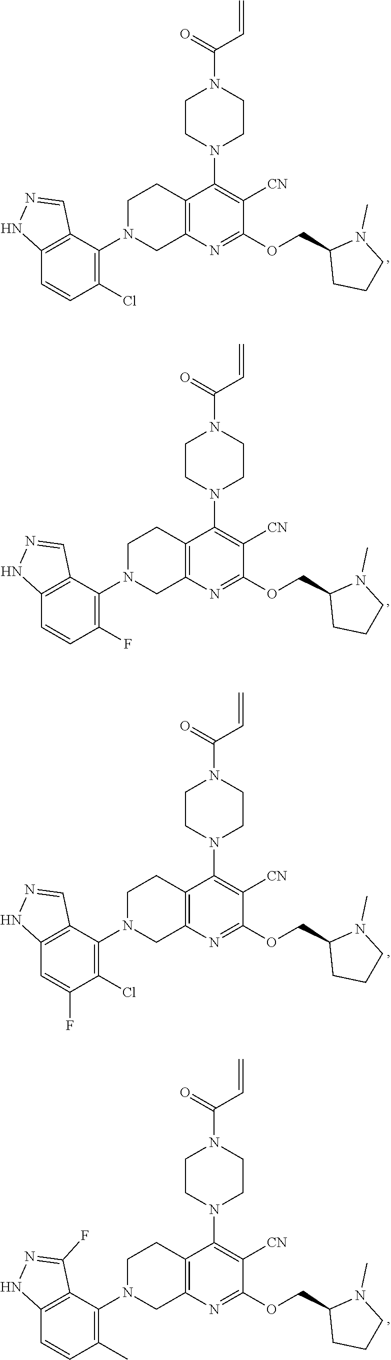 [SMILES: C=CC(=O)N1CCN(c2c(C#N)c(OC[C@@H]3CCCN3C)nc3c2CCN(c2c(Cl)ccc4[nH]ncc24)C3)CC1 ; C=CC(=O)N1CCN(c2c(C#N)c(OC[C@@H]3CCCN3C)nc3c2CCN(c2c(F)ccc4[nH]ncc24)C3)CC1 ; C=CC(=O)N1CCN(c2c(C#N)c(OC[C@@H]3CCCN3C)nc3c2CCN(c2c(Cl)c(F)cc4[nH]ncc24)C3)CC1 ; C=CC(=O)N1CCN(c2c(C#N)c(OC[C@@H]3CCCN3C)nc3c2CCN(c2c(C)ccc4[nH]nc(F)c24)C3)CC1]

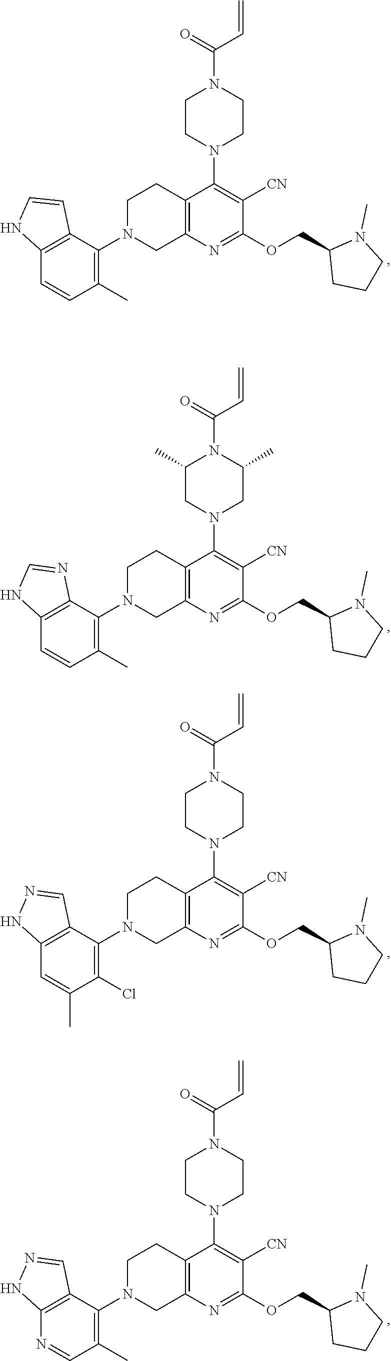 [SMILES: C=CC(=O)N1CCN(c2c(C#N)c(OC[C@@H]3CCCN3C)nc3c2CCN(c2c(C)ccc4[nH]ccc24)C3)CC1 ; C=CC(=O)N1[C@H](C)CN(c2c(C#N)c(OC[C@@H]3CCCN3C)nc3c2CCN(c2c(C)ccc4[nH]cnc24)C3)C[C@@H]1C ; C=CC(=O)N1CCN(c2c(C#N)c(OC[C@@H]3CCCN3C)nc3c2CCN(c2c(Cl)c(C)cc4[nH]ncc24)C3)CC1 ; C=CC(=O)N1CCN(c2c(C#N)c(OC[C@@H]3CCCN3C)nc3c2CCN(c2c(C)cnc4[nH]ncc24)C3)CC1]

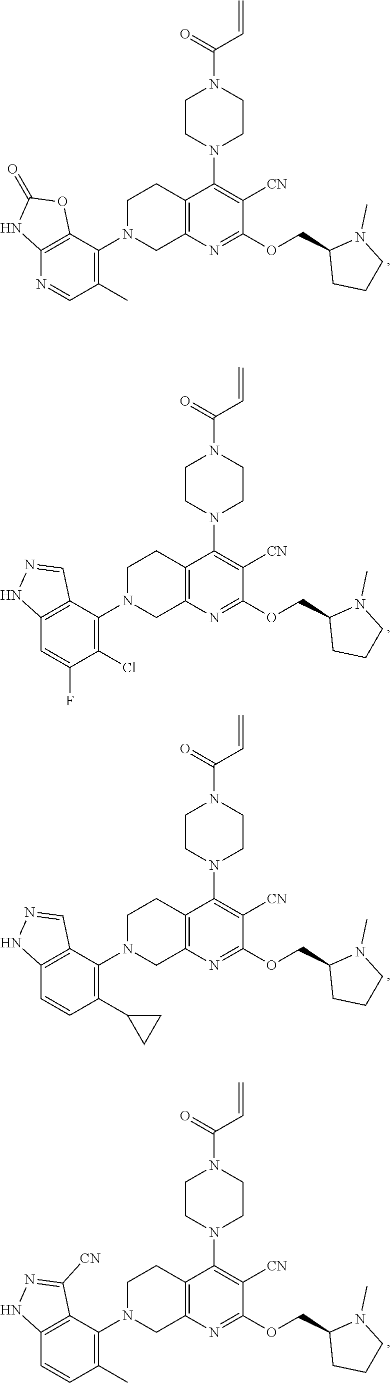 [SMILES: C=CC(=O)N1CCN(c2c(C#N)c(OC[C@@H]3CCCN3C)nc3c2CCN(c2c(C)cnc4[nH]c(=O)oc24)C3)CC1 ; C=CC(=O)N1CCN(c2c(C#N)c(OC[C@@H]3CCCN3C)nc3c2CCN(c2c(Cl)c(F)cc4[nH]ncc24)C3)CC1 ; C=CC(=O)N1CCN(c2c(C#N)c(OC[C@@H]3CCCN3C)nc3c2CCN(c2c(C4CC4)ccc4[nH]ncc24)C3)CC1 ; C=CC(=O)N1CCN(c2c(C#N)c(OC[C@@H]3CCCN3C)nc3c2CCN(c2c(C)ccc4[nH]nc(C#N)c24)C3)CC1]

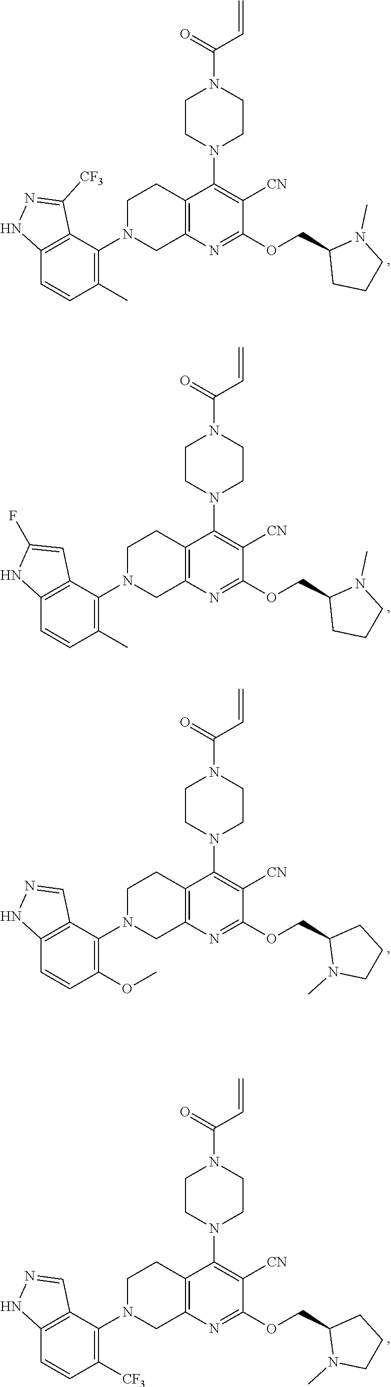 [SMILES: C=CC(=O)N1CCN(c2c(C#N)c(OC[C@@H]3CCCN3C)nc3c2CCN(c2c(C)ccc4[nH]nc(C(F)(F)F)c24)C3)CC1 ; C=CC(=O)N1CCN(c2c(C#N)c(OC[C@@H]3CCCN3C)nc3c2CCN(c2c(C)ccc4[nH]c(F)cc24)C3)CC1 ; C=CC(=O)N1CCN(c2c(C#N)c(OC[C@H]3CCCN3C)nc3c2CCN(c2c(OC)ccc4[nH]ncc24)C3)CC1 ; C=CC(=O)N1CCN(c2c(C#N)c(OC[C@H]3CCCN3C)nc3c2CCN(c2c(C(F)(F)F)ccc4[nH]ncc24)C3)CC1]

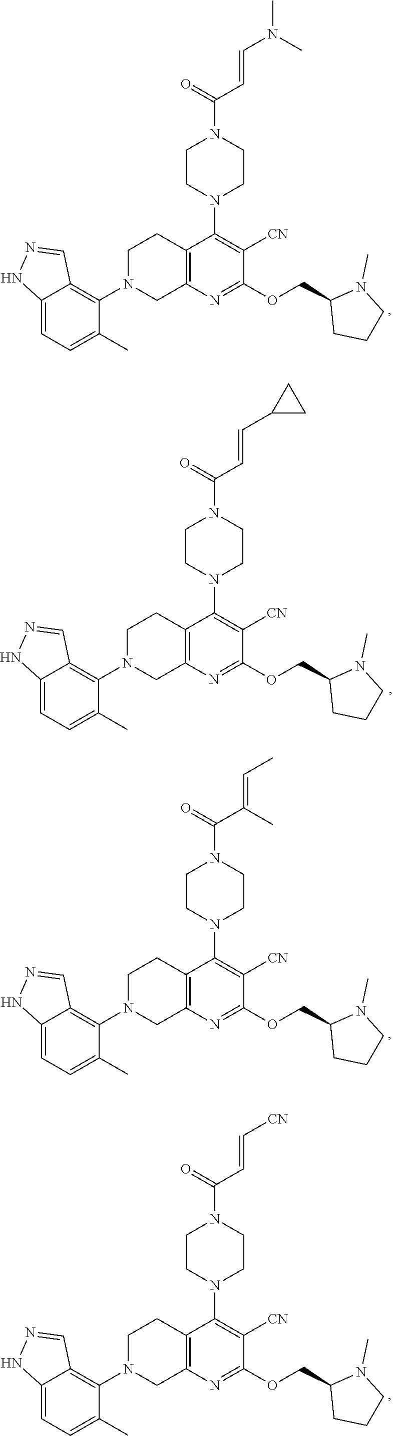 [SMILES: Cc1ccc2[nH]ncc2c1N1CCc2c(nc(OC[C@@H]3CCCN3C)c(C#N)c2N2CCN(C(=O)/C=C/N(C)C)CC2)C1 ; Cc1ccc2[nH]ncc2c1N1CCc2c(nc(OC[C@@H]3CCCN3C)c(C#N)c2N2CCN(C(=O)/C=C/C3CC3)CC2)C1 ; C/C=C(\C)C(=O)N1CCN(c2c(C#N)c(OC[C@@H]3CCCN3C)nc3c2CCN(c2c(C)ccc4[nH]ncc24)C3)CC1 ; Cc1ccc2[nH]ncc2c1N1CCc2c(nc(OC[C@@H]3CCCN3C)c(C#N)c2N2CCN(C(=O)/C=C/C#N)CC2)C1]

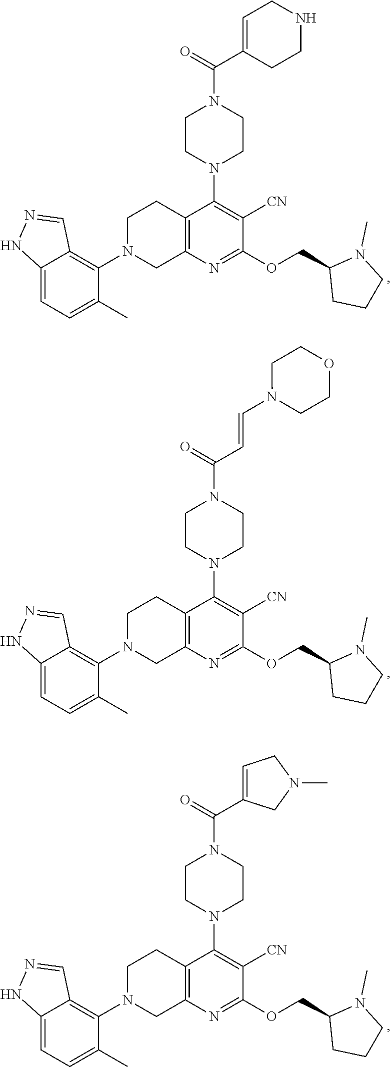 [SMILES: Cc1ccc2[nH]ncc2c1N1CCc2c(nc(OC[C@@H]3CCCN3C)c(C#N)c2N2CCN(C(=O)C3=CCNCC3)CC2)C1 ; Cc1ccc2[nH]ncc2c1N1CCc2c(nc(OC[C@@H]3CCCN3C)c(C#N)c2N2CCN(C(=O)/C=C/N3CCOCC3)CC2)C1 ; Cc1ccc2[nH]ncc2c1N1CCc2c(nc(OC[C@@H]3CCCN3C)c(C#N)c2N2CCN(C(=O)C3=CCN(C)C3)CC2)C1]

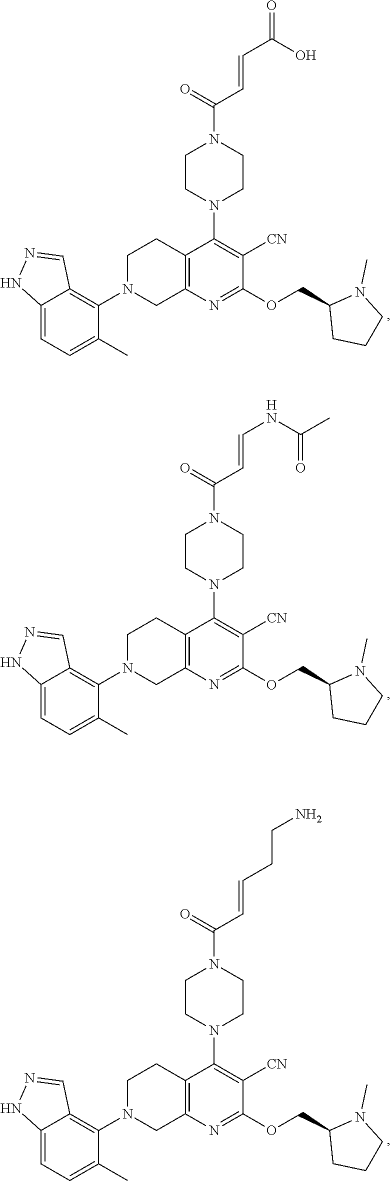

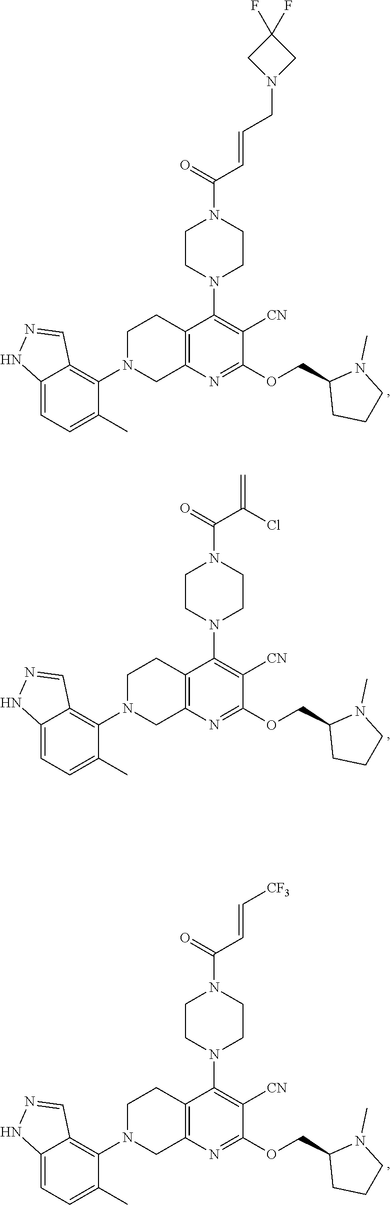 [SMILES: Cc1ccc2[nH]ncc2c1N1CCc2c(nc(OC[C@@H]3CCCN3C)c(C#N)c2N2CCN(C(=O)/C=C/CN3CC(F)(F)C3)CC2)C1 ; C=C(Cl)C(=O)N1CCN(c2c(C#N)c(OC[C@@H]3CCCN3C)nc3c2CCN(c2c(C)ccc4[nH]ncc24)C3)CC1 ; Cc1ccc2[nH]ncc2c1N1CCc2c(nc(OC[C@@H]3CCCN3C)c(C#N)c2N2CCN(C(=O)/C=C/C(F)(F)F)CC2)C1]

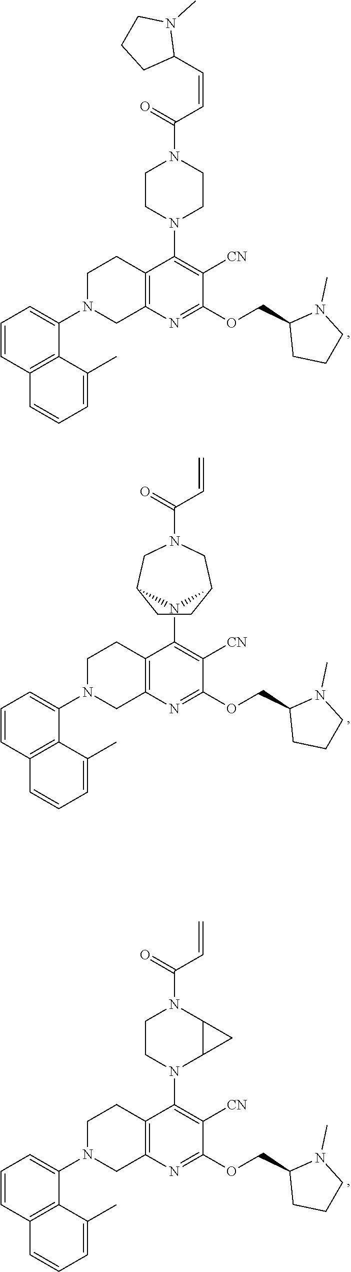

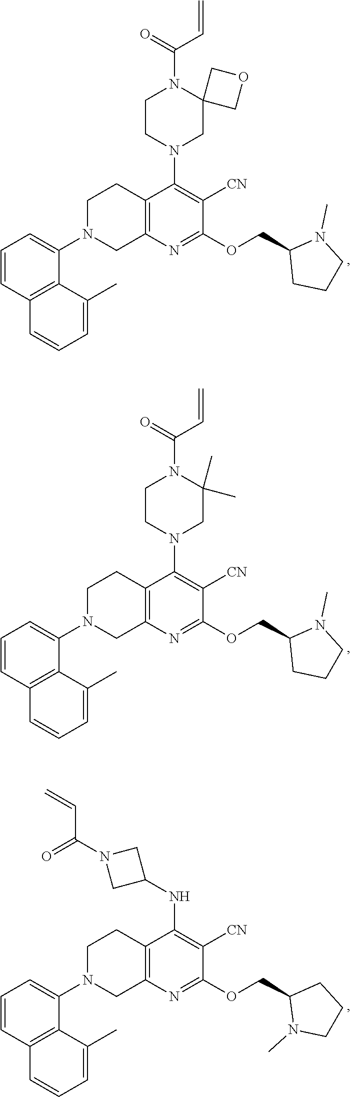 [SMILES: C=CC(=O)N1CCN(c2c(C#N)c(OC[C@@H]3CCCN3C)nc3c2CCN(c2cccc4cccc(C)c24)C3)CC12COC2 ; C=CC(=O)N1CCN(c2c(C#N)c(OC[C@@H]3CCCN3C)nc3c2CCN(c2cccc4cccc(C)c24)C3)CC1(C)C ; C=CC(=O)N1CC(Nc2c(C#N)c(OC[C@H]3CCCN3C)nc3c2CCN(c2cccc4cccc(C)c24)C3)C1]

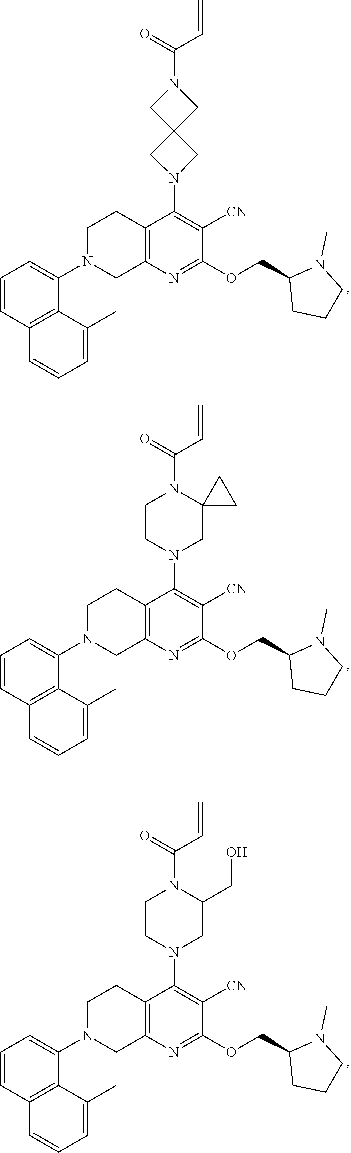 [SMILES: C=CC(=O)N1CC2(C1)CN(c1c(C#N)c(OC[C@@H]3CCCN3C)nc3c1CCN(c1cccc4cccc(C)c14)C3)C2 ; C=CC(=O)N1CCN(c2c(C#N)c(OC[C@@H]3CCCN3C)nc3c2CCN(c2cccc4cccc(C)c24)C3)CC12CC2 ; C=CC(=O)N1CCN(c2c(C#N)c(OC[C@@H]3CCCN3C)nc3c2CCN(c2cccc4cccc(C)c24)C3)CC1CO]

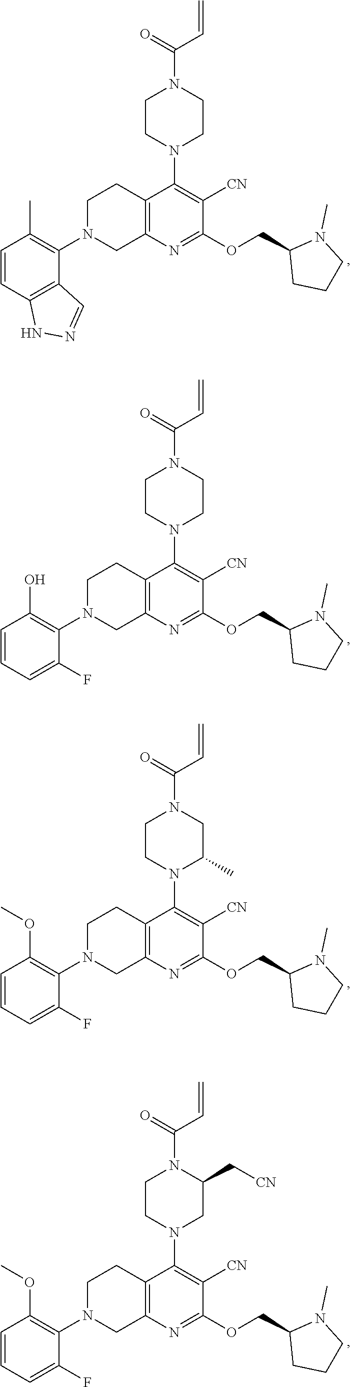 [SMILES: C=CC(=O)N1CCN(c2c(C#N)c(OC[C@@H]3CCCN3C)nc3c2CCN(c2c(C)ccc4[nH]ncc24)C3)CC1 ; C=CC(=O)N1CCN(c2c(C#N)c(OC[C@@H]3CCCN3C)nc3c2CCN(c2c(O)cccc2F)C3)CC1 ; C=CC(=O)N1CCN(c2c(C#N)c(OC[C@@H]3CCCN3C)nc3c2CCN(c2c(F)cccc2OC)C3)[C@@H](C)C1 ; C=CC(=O)N1CCN(c2c(C#N)c(OC[C@@H]3CCCN3C)nc3c2CCN(c2c(F)cccc2OC)C3)C[C@@H]1CC#N]

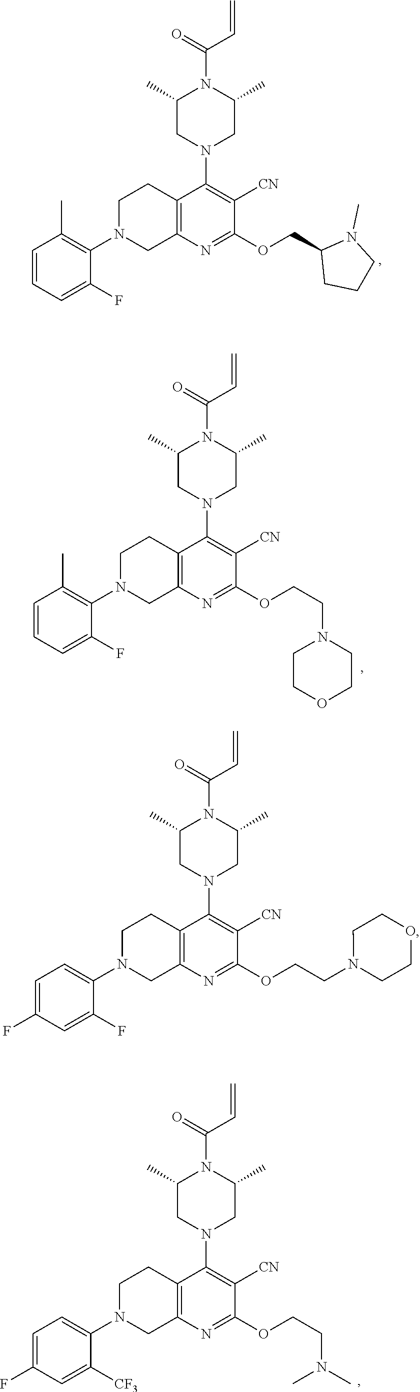 [SMILES: C=CC(=O)N1[C@H](C)CN(c2c(C#N)c(OC[C@@H]3CCCN3C)nc3c2CCN(c2c(C)cccc2F)C3)C[C@@H]1C ; C=CC(=O)N1[C@H](C)CN(c2c(C#N)c(OCCN3CCOCC3)nc3c2CCN(c2c(C)cccc2F)C3)C[C@@H]1C ; C=CC(=O)N1[C@H](C)CN(c2c(C#N)c(OCCN3CCOCC3)nc3c2CCN(c2ccc(F)cc2F)C3)C[C@@H]1C ; C=CC(=O)N1[C@H](C)CN(c2c(C#N)c(OCCN(C)C)nc3c2CCN(c2ccc(F)cc2C(F)(F)F)C3)C[C@@H]1C]

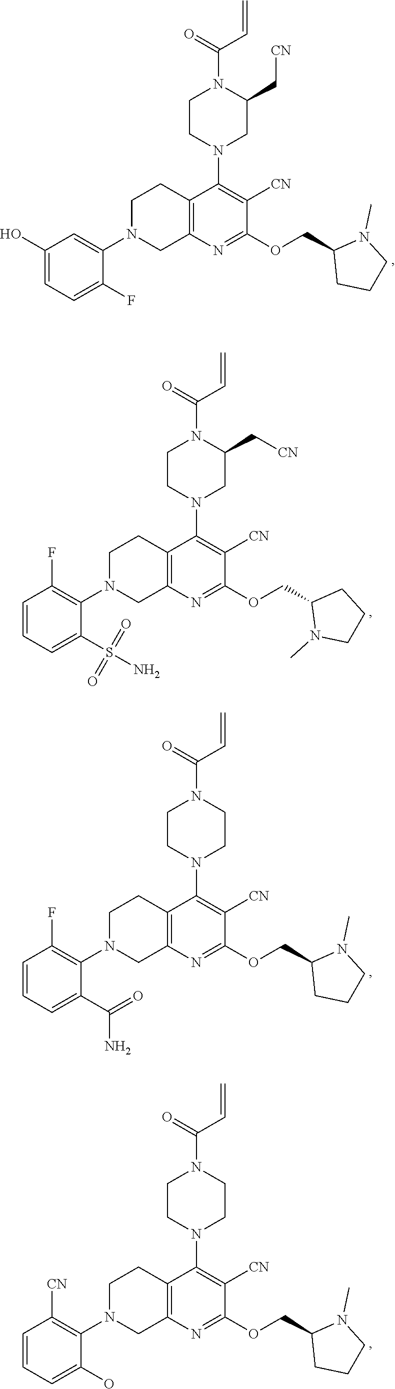 [SMILES: C=CC(=O)N1CCN(c2c(C#N)c(OC[C@@H]3CCCN3C)nc3c2CCN(c2cc(O)ccc2F)C3)C[C@@H]1CC#N ; C=CC(=O)N1CCN(c2c(C#N)c(OC[C@@H]3CCCN3C)nc3c2CCN(c2c(F)cccc2S(N)(=O)=O)C3)C[C@@H]1CC#N ; C=CC(=O)N1CCN(c2c(C#N)c(OC[C@@H]3CCCN3C)nc3c2CCN(c2c(F)cccc2C(N)=O)C3)CC1 ; C=CC(=O)N1CCN(c2c(C#N)c(OC[C@@H]3CCCN3C)nc3c2CCN(c2c(O)cccc2C#N)C3)CC1]

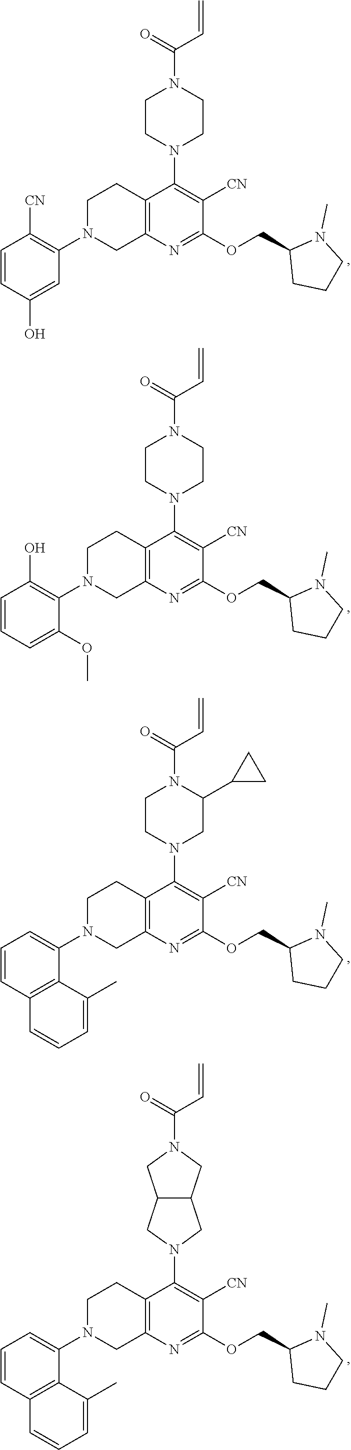 [SMILES: C=CC(=O)N1CCN(c2c(C#N)c(OC[C@@H]3CCCN3C)nc3c2CCN(c2cc(O)ccc2C#N)C3)CC1 ; C=CC(=O)N1CCN(c2c(C#N)c(OC[C@@H]3CCCN3C)nc3c2CCN(c2c(O)cccc2OC)C3)CC1 ; C=CC(=O)N1CCN(c2c(C#N)c(OC[C@@H]3CCCN3C)nc3c2CCN(c2cccc4cccc(C)c24)C3)CC1C1CC1 ; C=CC(=O)N1CC2CN(c3c(C#N)c(OC[C@@H]4CCCN4C)nc4c3CCN(c3cccc5cccc(C)c35)C4)CC2C1]

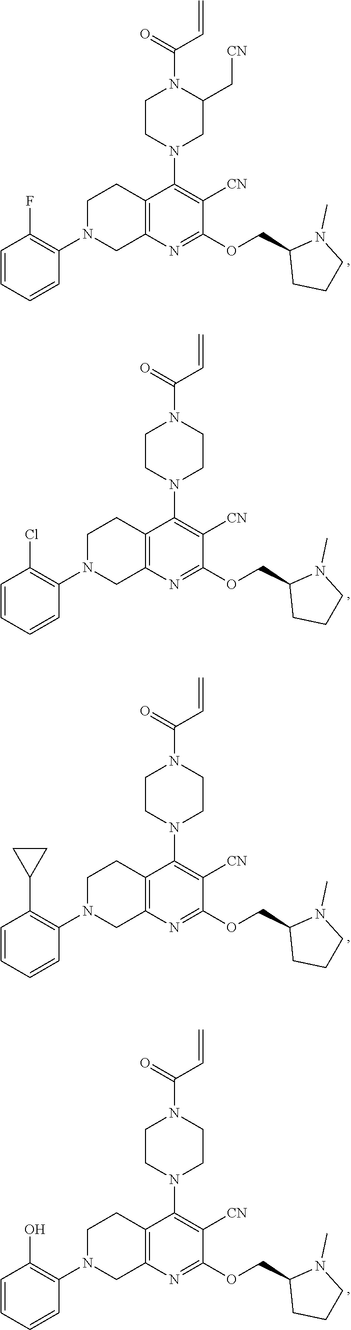 [SMILES: C=CC(=O)N1CCN(c2c(C#N)c(OC[C@@H]3CCCN3C)nc3c2CCN(c2ccccc2F)C3)CC1CC#N ; C=CC(=O)N1CCN(c2c(C#N)c(OC[C@@H]3CCCN3C)nc3c2CCN(c2ccccc2Cl)C3)CC1 ; C=CC(=O)N1CCN(c2c(C#N)c(OC[C@@H]3CCCN3C)nc3c2CCN(c2ccccc2C2CC2)C3)CC1 ; C=CC(=O)N1CCN(c2c(C#N)c(OC[C@@H]3CCCN3C)nc3c2CCN(c2ccccc2O)C3)CC1]

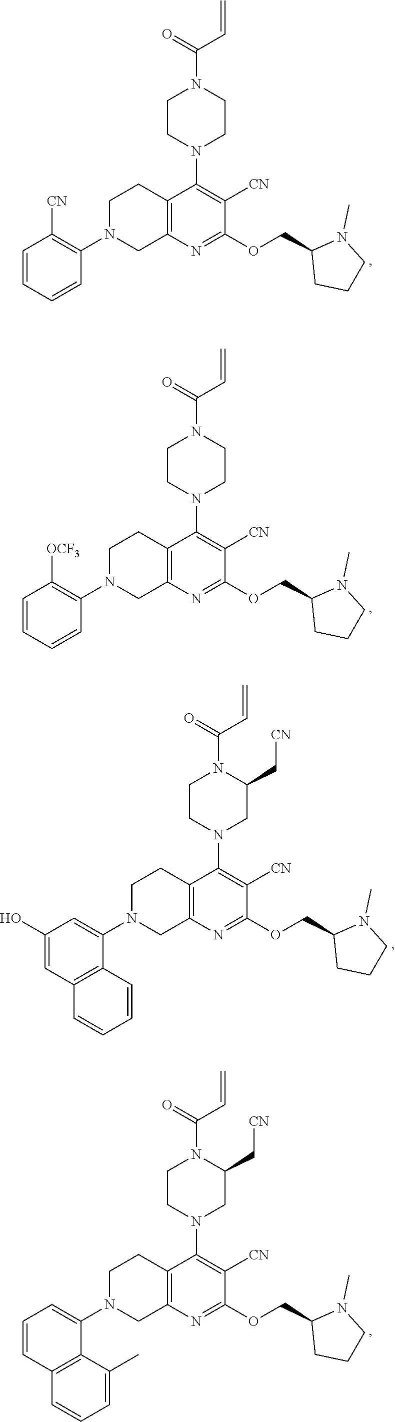 [SMILES: C=CC(=O)N1CCN(c2c(C#N)c(OC[C@@H]3CCCN3C)nc3c2CCN(c2ccccc2C#N)C3)CC1 ; C=CC(=O)N1CCN(c2c(C#N)c(OC[C@@H]3CCCN3C)nc3c2CCN(c2ccccc2OC(F)(F)F)C3)CC1 ; C=CC(=O)N1CCN(c2c(C#N)c(OC[C@@H]3CCCN3C)nc3c2CCN(c2cc(O)cc4ccccc24)C3)C[C@@H]1CC#N ; C=CC(=O)N1CCN(c2c(C#N)c(OC[C@@H]3CCCN3C)nc3c2CCN(c2cccc4cccc(C)c24)C3)C[C@@H]1CC#N]

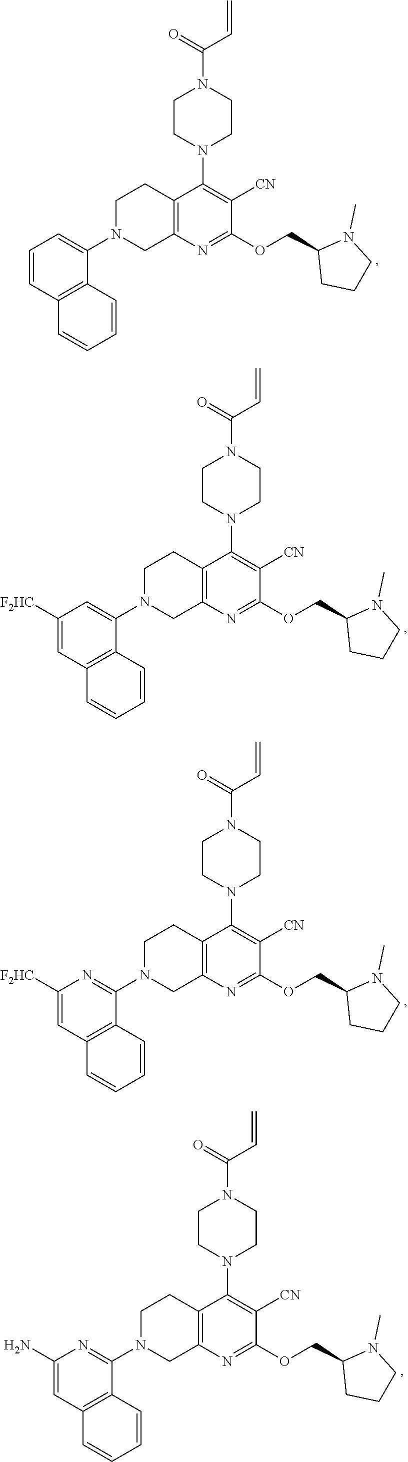 [SMILES: C=CC(=O)N1CCN(c2c(C#N)c(OC[C@@H]3CCCN3C)nc3c2CCN(c2cccc4ccccc24)C3)CC1 ; C=CC(=O)N1CCN(c2c(C#N)c(OC[C@@H]3CCCN3C)nc3c2CCN(c2cc(C(F)F)cc4ccccc24)C3)CC1 ; C=CC(=O)N1CCN(c2c(C#N)c(OC[C@@H]3CCCN3C)nc3c2CCN(c2nc(C(F)F)cc4ccccc24)C3)CC1 ; C=CC(=O)N1CCN(c2c(C#N)c(OC[C@@H]3CCCN3C)nc3c2CCN(c2nc(N)cc4ccccc24)C3)CC1]

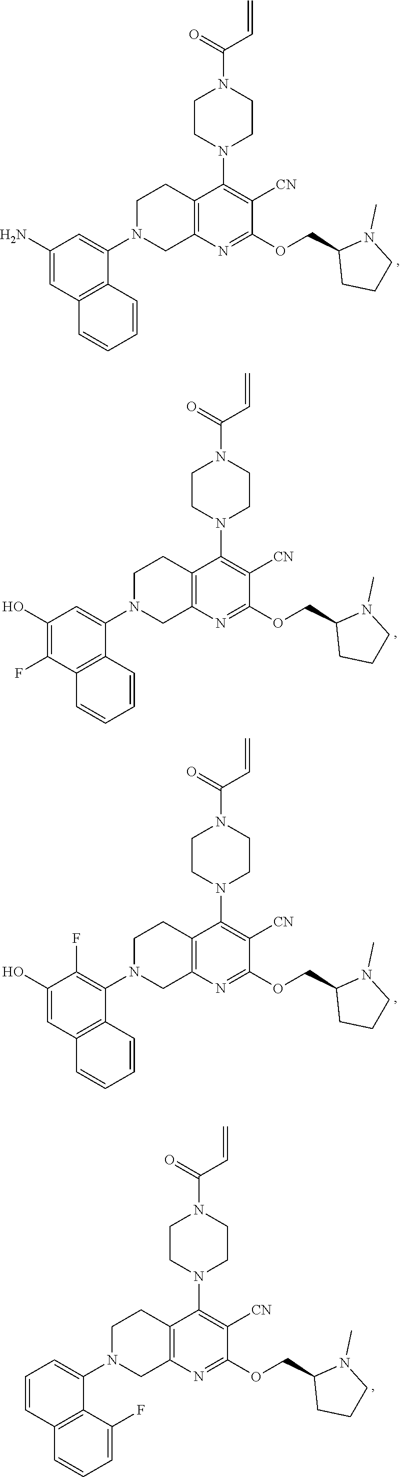 [SMILES: C=CC(=O)N1CCN(c2c(C#N)c(OC[C@@H]3CCCN3C)nc3c2CCN(c2cc(N)cc4ccccc24)C3)CC1 ; C=CC(=O)N1CCN(c2c(C#N)c(OC[C@@H]3CCCN3C)nc3c2CCN(c2cc(O)c(F)c4ccccc24)C3)CC1 ; C=CC(=O)N1CCN(c2c(C#N)c(OC[C@@H]3CCCN3C)nc3c2CCN(c2c(F)c(O)cc4ccccc24)C3)CC1 ; C=CC(=O)N1CCN(c2c(C#N)c(OC[C@@H]3CCCN3C)nc3c2CCN(c2cccc4cccc(F)c24)C3)CC1]

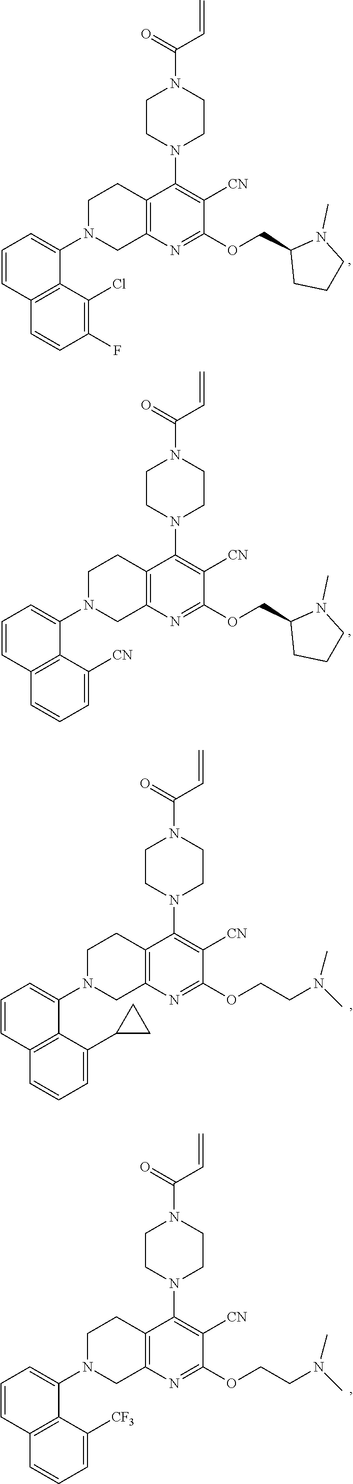 [SMILES: C=CC(=O)N1CCN(c2c(C#N)c(OC[C@@H]3CCCN3C)nc3c2CCN(c2cccc4ccc(F)c(Cl)c24)C3)CC1 ; C=CC(=O)N1CCN(c2c(C#N)c(OC[C@@H]3CCCN3C)nc3c2CCN(c2cccc4cccc(C#N)c24)C3)CC1 ; C=CC(=O)N1CCN(c2c(C#N)c(OCCN(C)C)nc3c2CCN(c2cccc4cccc(C5CC5)c24)C3)CC1 ; C=CC(=O)N1CCN(c2c(C#N)c(OCCN(C)C)nc3c2CCN(c2cccc4cccc(C(F)(F)F)c24)C3)CC1]

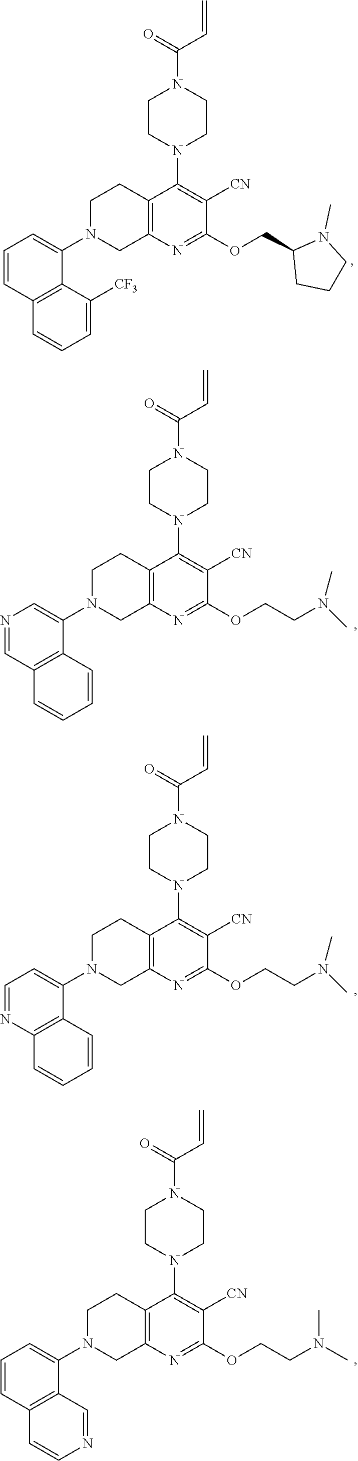 [SMILES: C=CC(=O)N1CCN(c2c(C#N)c(OC[C@@H]3CCCN3C)nc3c2CCN(c2cccc4cccc(C(F)(F)F)c24)C3)CC1 ; C=CC(=O)N1CCN(c2c(C#N)c(OCCN(C)C)nc3c2CCN(c2cncc4ccccc24)C3)CC1 ; C=CC(=O)N1CCN(c2c(C#N)c(OCCN(C)C)nc3c2CCN(c2ccnc4ccccc24)C3)CC1 ; C=CC(=O)N1CCN(c2c(C#N)c(OCCN(C)C)nc3c2CCN(c2cccc4ccncc24)C3)CC1]

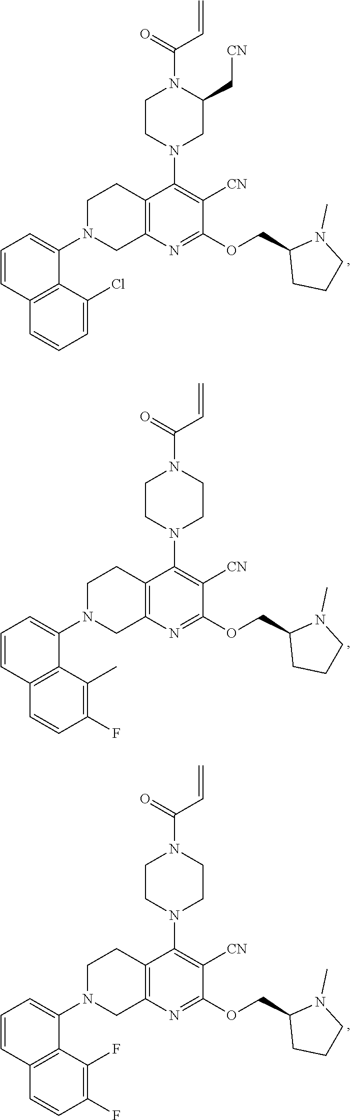 [SMILES: C=CC(=O)N1CCN(c2c(C#N)c(OC[C@@H]3CCCN3C)nc3c2CCN(c2cccc4cccc(Cl)c24)C3)C[C@@H]1CC#N ; C=CC(=O)N1CCN(c2c(C#N)c(OC[C@@H]3CCCN3C)nc3c2CCN(c2cccc4ccc(F)c(C)c24)C3)CC1 ; C=CC(=O)N1CCN(c2c(C#N)c(OC[C@@H]3CCCN3C)nc3c2CCN(c2cccc4ccc(F)c(F)c24)C3)CC1]

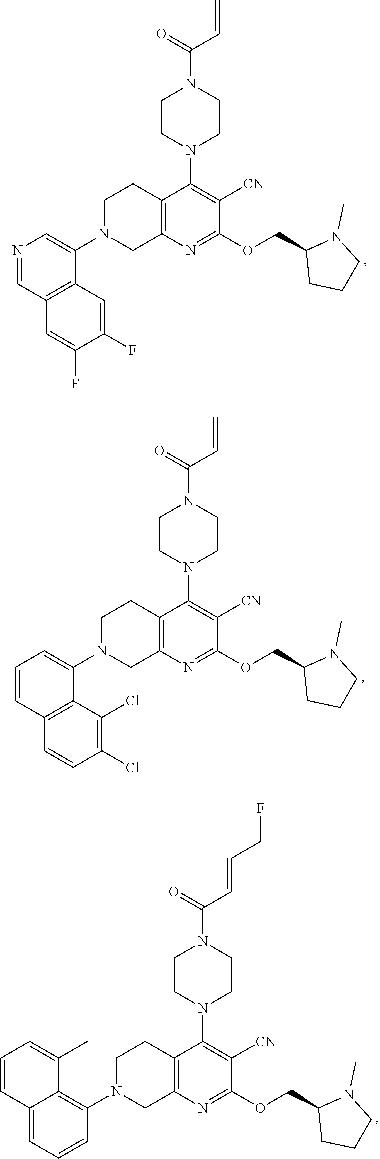 [SMILES: C=CC(=O)N1CCN(c2c(C#N)c(OC[C@@H]3CCCN3C)nc3c2CCN(c2cncc4cc(F)c(F)cc24)C3)CC1 ; C=CC(=O)N1CCN(c2c(C#N)c(OC[C@@H]3CCCN3C)nc3c2CCN(c2cccc4ccc(Cl)c(Cl)c24)C3)CC1 ; Cc1cccc2cccc(N3CCc4c(nc(OC[C@@H]5CCCN5C)c(C#N)c4N4CCN(C(=O)/C=C/CF)CC4)C3)c12]

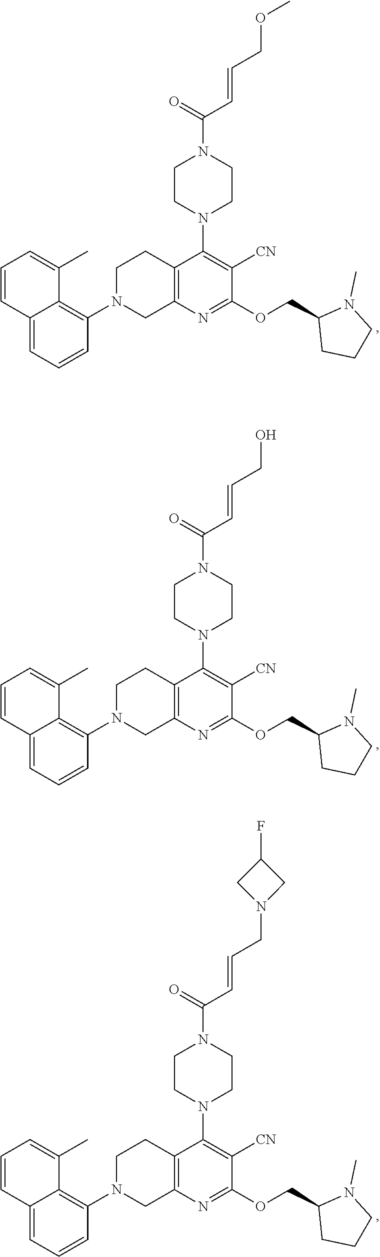 [SMILES: COC/C=C/C(=O)N1CCN(c2c(C#N)c(OC[C@@H]3CCCN3C)nc3c2CCN(c2cccc4cccc(C)c24)C3)CC1 ; Cc1cccc2cccc(N3CCc4c(nc(OC[C@@H]5CCCN5C)c(C#N)c4N4CCN(C(=O)/C=C/CO)CC4)C3)c12 ; Cc1cccc2cccc(N3CCc4c(nc(OC[C@@H]5CCCN5C)c(C#N)c4N4CCN(C(=O)/C=C/CN5CC(F)C5)CC4)C3)c12]

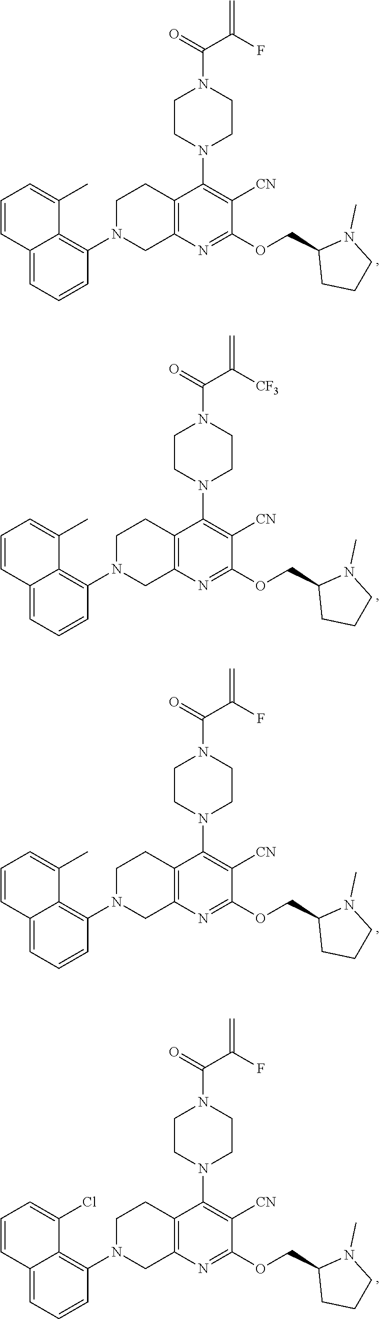 [SMILES: C=C(F)C(=O)N1CCN(c2c(C#N)c(OC[C@@H]3CCCN3C)nc3c2CCN(c2cccc4cccc(C)c24)C3)CC1 ; C=C(C(=O)N1CCN(c2c(C#N)c(OC[C@@H]3CCCN3C)nc3c2CCN(c2cccc4cccc(C)c24)C3)CC1)C(F)(F)F ; C=C(F)C(=O)N1CCN(c2c(C#N)c(OC[C@@H]3CCCN3C)nc3c2CCN(c2cccc4cccc(C)c24)C3)CC1 ; C=C(F)C(=O)N1CCN(c2c(C#N)c(OC[C@@H]3CCCN3C)nc3c2CCN(c2cccc4cccc(Cl)c24)C3)CC1]

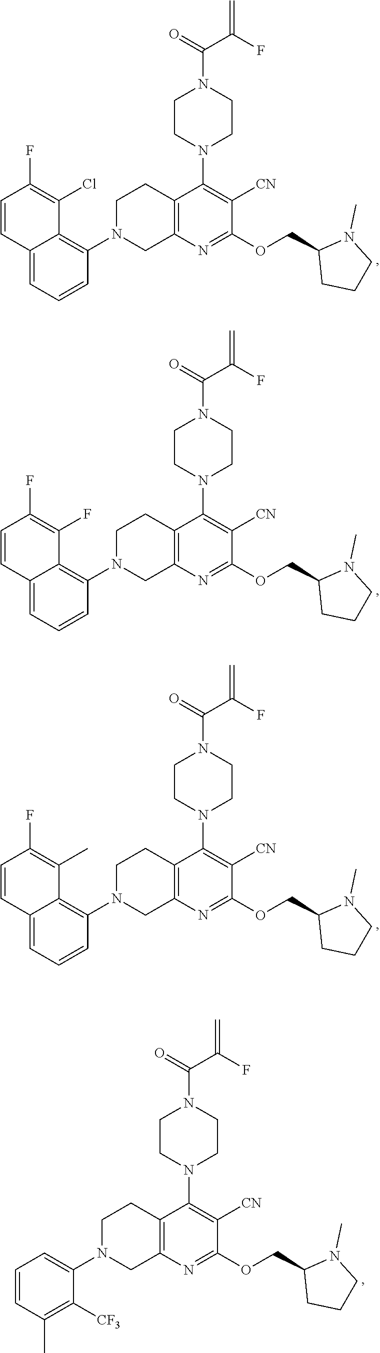 [SMILES: C=C(F)C(=O)N1CCN(c2c(C#N)c(OC[C@@H]3CCCN3C)nc3c2CCN(c2cccc4ccc(F)c(Cl)c24)C3)CC1 ; C=C(F)C(=O)N1CCN(c2c(C#N)c(OC[C@@H]3CCCN3C)nc3c2CCN(c2cccc4ccc(F)c(F)c24)C3)CC1 ; C=C(F)C(=O)N1CCN(c2c(C#N)c(OC[C@@H]3CCCN3C)nc3c2CCN(c2cccc4ccc(F)c(C)c24)C3)CC1 ; C=C(F)C(=O)N1CCN(c2c(C#N)c(OC[C@@H]3CCCN3C)nc3c2CCN(c2cccc(C)c2C(F)(F)F)C3)CC1]

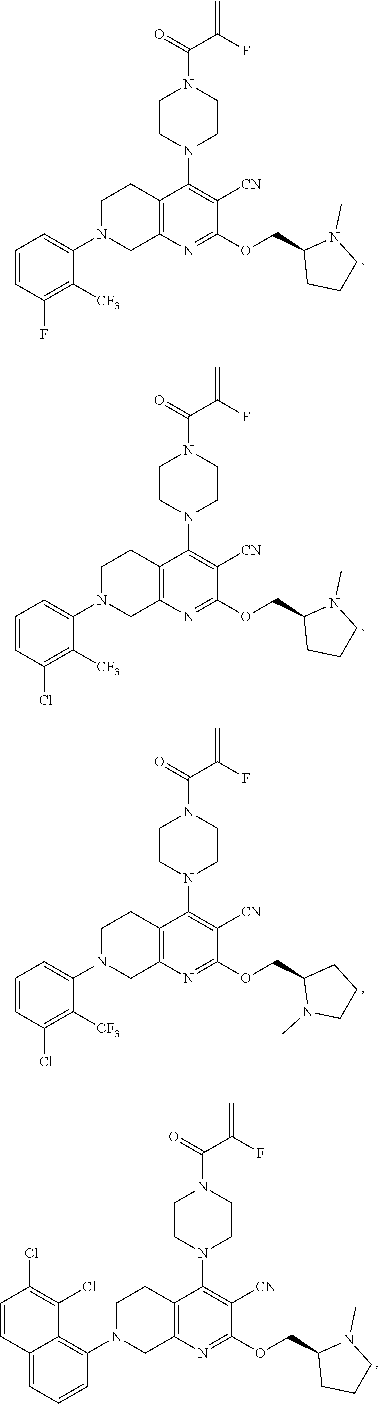 [SMILES: C=C(F)C(=O)N1CCN(c2c(C#N)c(OC[C@@H]3CCCN3C)nc3c2CCN(c2cccc(F)c2C(F)(F)F)C3)CC1 ; C=C(F)C(=O)N1CCN(c2c(C#N)c(OC[C@@H]3CCCN3C)nc3c2CCN(c2cccc(Cl)c2C(F)(F)F)C3)CC1 ; C=C(F)C(=O)N1CCN(c2c(C#N)c(OC[C@H]3CCCN3C)nc3c2CCN(c2cccc(Cl)c2C(F)(F)F)C3)CC1 ; C=C(F)C(=O)N1CCN(c2c(C#N)c(OC[C@@H]3CCCN3C)nc3c2CCN(c2cccc4ccc(Cl)c(Cl)c24)C3)CC1]

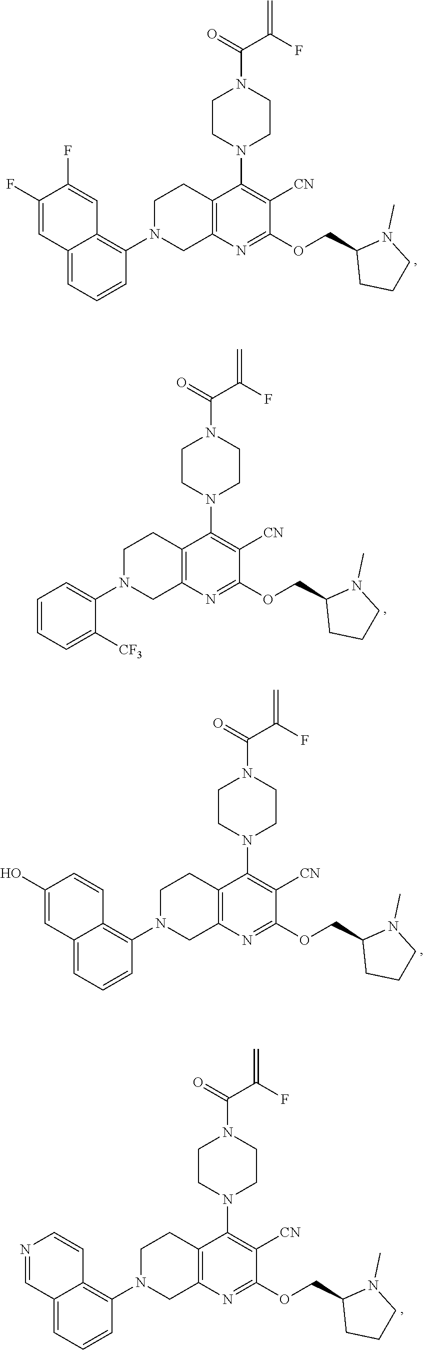 [SMILES: C=C(F)C(=O)N1CCN(c2c(C#N)c(OC[C@@H]3CCCN3C)nc3c2CCN(c2cccc4cc(F)c(F)cc24)C3)CC1 ; C=C(F)C(=O)N1CCN(c2c(C#N)c(OC[C@@H]3CCCN3C)nc3c2CCN(c2ccccc2C(F)(F)F)C3)CC1 ; C=C(F)C(=O)N1CCN(c2c(C#N)c(OC[C@@H]3CCCN3C)nc3c2CCN(c2cccc4cc(O)ccc24)C3)CC1 ; C=C(F)C(=O)N1CCN(c2c(C#N)c(OC[C@@H]3CCCN3C)nc3c2CCN(c2cccc4cnccc24)C3)CC1]

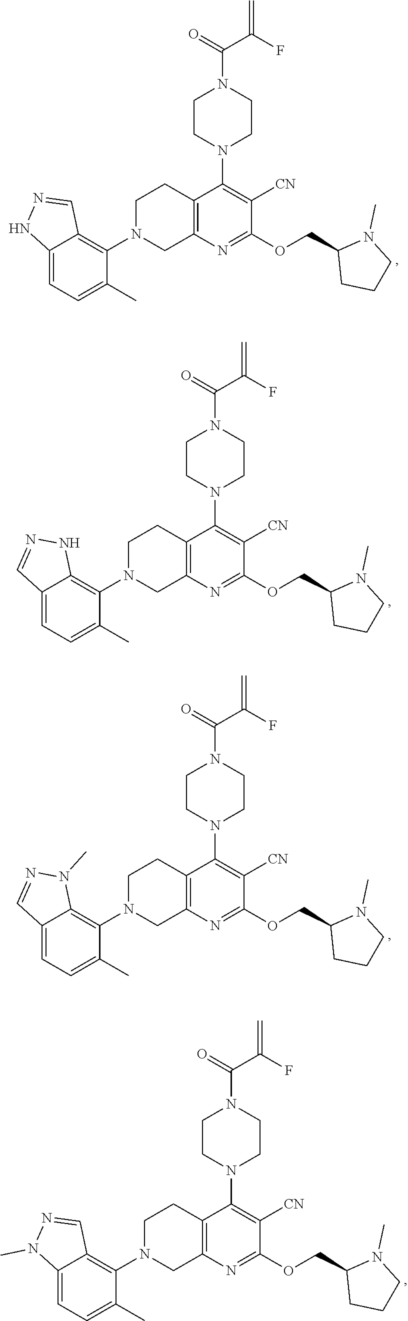 [SMILES: C=C(F)C(=O)N1CCN(c2c(C#N)c(OC[C@@H]3CCCN3C)nc3c2CCN(c2c(C)ccc4[nH]ncc24)C3)CC1 ; C=C(F)C(=O)N1CCN(c2c(C#N)c(OC[C@@H]3CCCN3C)nc3c2CCN(c2c(C)ccc4cn[nH]c24)C3)CC1 ; C=C(F)C(=O)N1CCN(c2c(C#N)c(OC[C@@H]3CCCN3C)nc3c2CCN(c2c(C)ccc4cnn(C)c24)C3)CC1 ; C=C(F)C(=O)N1CCN(c2c(C#N)c(OC[C@@H]3CCCN3C)nc3c2CCN(c2c(C)ccc4c2cnn4C)C3)CC1]

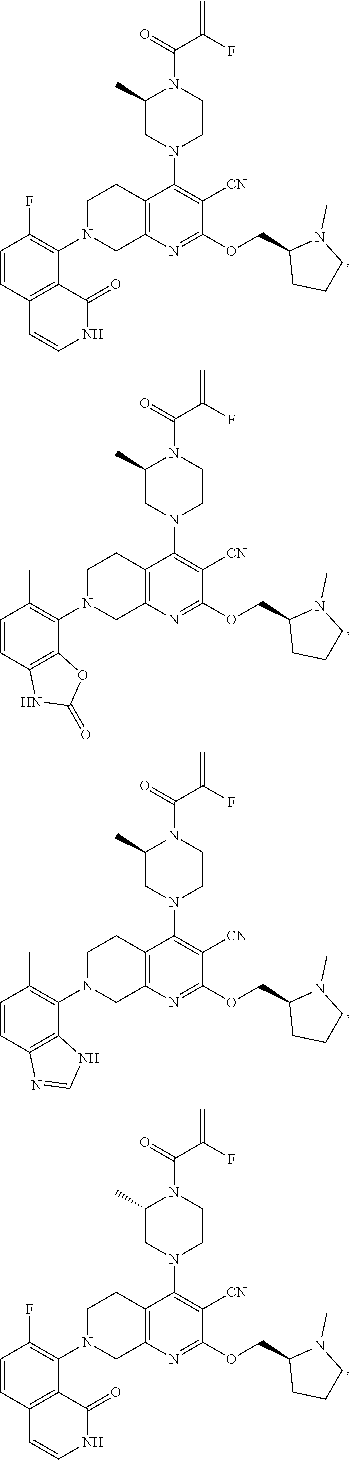 [SMILES: C=C(F)C(=O)N1CCN(c2c(C#N)c(OC[C@@H]3CCCN3C)nc3c2CCN(c2c(F)ccc4cc[nH]c(=O)c24)C3)C[C@H]1C ; C=C(F)C(=O)N1CCN(c2c(C#N)c(OC[C@@H]3CCCN3C)nc3c2CCN(c2c(C)ccc4[nH]c(=O)oc24)C3)C[C@H]1C ; C=C(F)C(=O)N1CCN(c2c(C#N)c(OC[C@@H]3CCCN3C)nc3c2CCN(c2c(C)ccc4nc[nH]c24)C3)C[C@H]1C ; C=C(F)C(=O)N1CCN(c2c(C#N)c(OC[C@@H]3CCCN3C)nc3c2CCN(c2c(F)ccc4cc[nH]c(=O)c24)C3)C[C@@H]1C]

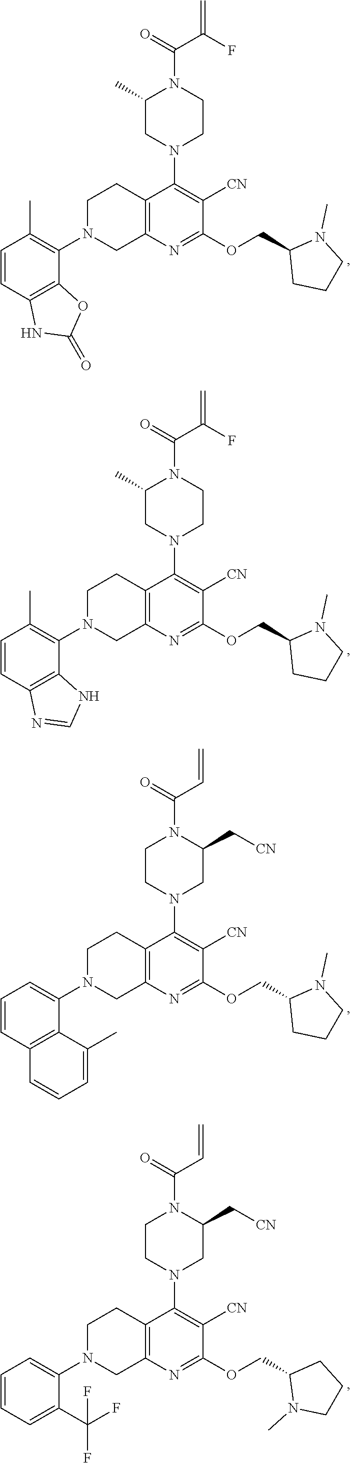 [SMILES: C=C(F)C(=O)N1CCN(c2c(C#N)c(OC[C@@H]3CCCN3C)nc3c2CCN(c2c(C)ccc4[nH]c(=O)oc24)C3)C[C@@H]1C ; C=C(F)C(=O)N1CCN(c2c(C#N)c(OC[C@@H]3CCCN3C)nc3c2CCN(c2c(C)ccc4nc[nH]c24)C3)C[C@@H]1C ; C=CC(=O)N1CCN(c2c(C#N)c(OC[C@H]3CCCN3C)nc3c2CCN(c2cccc4cccc(C)c24)C3)C[C@@H]1CC#N ; C=CC(=O)N1CCN(c2c(C#N)c(OC[C@@H]3CCCN3C)nc3c2CCN(c2ccccc2C(F)(F)F)C3)C[C@@H]1CC#N]

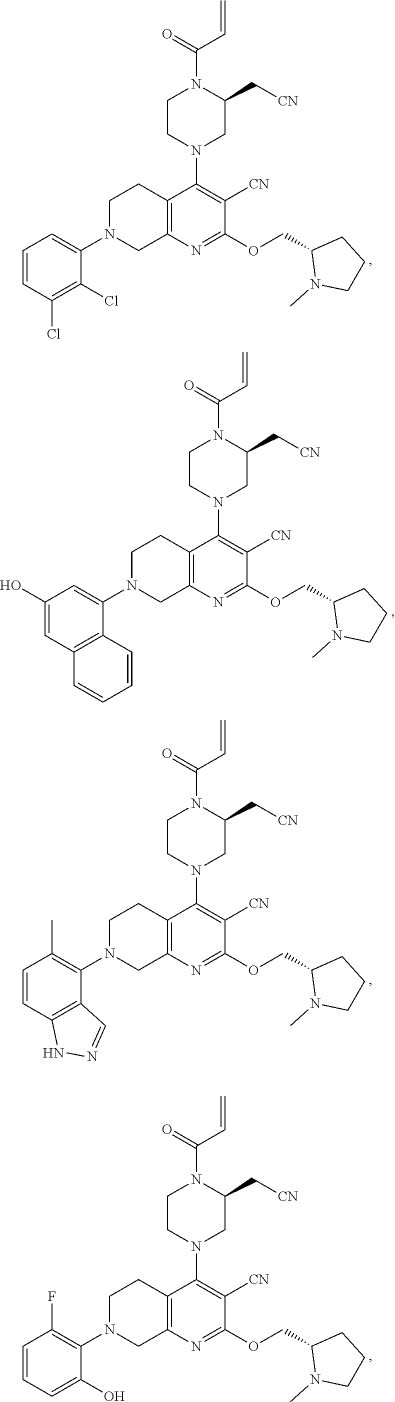 [SMILES: C=CC(=O)N1CCN(c2c(C#N)c(OC[C@@H]3CCCN3C)nc3c2CCN(c2cccc(Cl)c2Cl)C3)C[C@@H]1CC#N ; C=CC(=O)N1CCN(c2c(C#N)c(OC[C@@H]3CCCN3C)nc3c2CCN(c2cc(O)cc4ccccc24)C3)C[C@@H]1CC#N ; C=CC(=O)N1CCN(c2c(C#N)c(OC[C@@H]3CCCN3C)nc3c2CCN(c2c(C)ccc4[nH]ncc24)C3)C[C@@H]1CC#N ; C=CC(=O)N1CCN(c2c(C#N)c(OC[C@@H]3CCCN3C)nc3c2CCN(c2c(O)cccc2F)C3)C[C@@H]1CC#N]

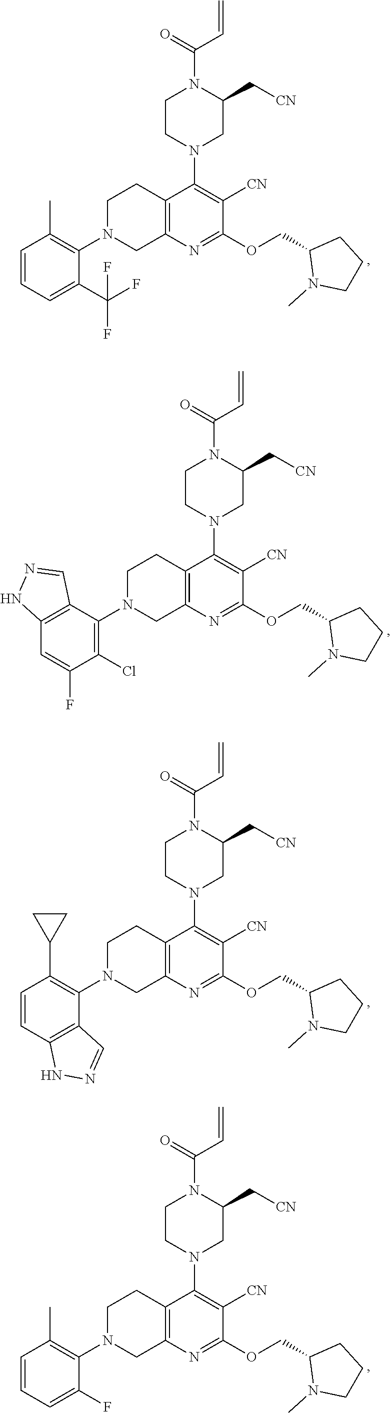 [SMILES: C=CC(=O)N1CCN(c2c(C#N)c(OC[C@@H]3CCCN3C)nc3c2CCN(c2c(C)cccc2C(F)(F)F)C3)C[C@@H]1CC#N ; C=CC(=O)N1CCN(c2c(C#N)c(OC[C@@H]3CCCN3C)nc3c2CCN(c2c(Cl)c(F)cc4[nH]ncc24)C3)C[C@@H]1CC#N ; C=CC(=O)N1CCN(c2c(C#N)c(OC[C@@H]3CCCN3C)nc3c2CCN(c2c(C4CC4)ccc4[nH]ncc24)C3)C[C@@H]1CC#N ; C=CC(=O)N1CCN(c2c(C#N)c(OC[C@@H]3CCCN3C)nc3c2CCN(c2c(C)cccc2F)C3)C[C@@H]1CC#N]

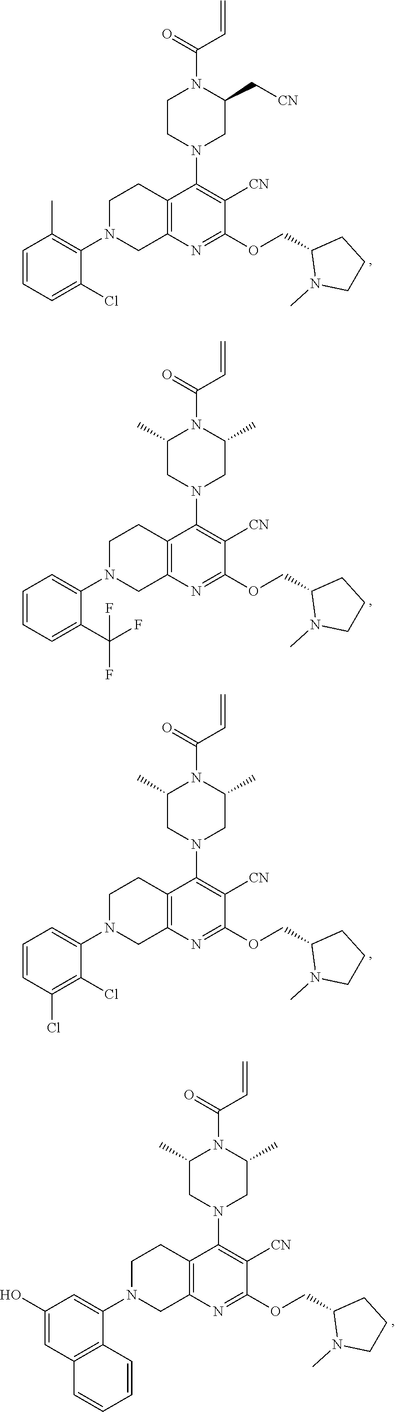 [SMILES: C=CC(=O)N1CCN(c2c(C#N)c(OC[C@@H]3CCCN3C)nc3c2CCN(c2c(C)cccc2Cl)C3)C[C@@H]1CC#N ; C=CC(=O)N1[C@H](C)CN(c2c(C#N)c(OC[C@@H]3CCCN3C)nc3c2CCN(c2ccccc2C(F)(F)F)C3)C[C@@H]1C ; C=CC(=O)N1[C@H](C)CN(c2c(C#N)c(OC[C@@H]3CCCN3C)nc3c2CCN(c2cccc(Cl)c2Cl)C3)C[C@@H]1C ; C=CC(=O)N1[C@H](C)CN(c2c(C#N)c(OC[C@@H]3CCCN3C)nc3c2CCN(c2cc(O)cc4ccccc24)C3)C[C@@H]1C]

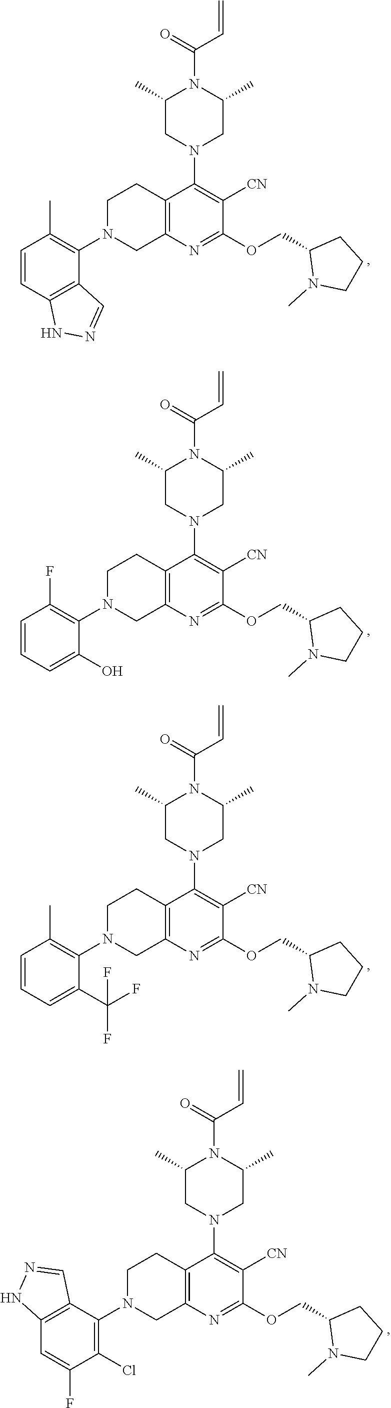 [SMILES: C=CC(=O)N1[C@H](C)CN(c2c(C#N)c(OC[C@@H]3CCCN3C)nc3c2CCN(c2c(C)ccc4[nH]ncc24)C3)C[C@@H]1C ; C=CC(=O)N1[C@H](C)CN(c2c(C#N)c(OC[C@@H]3CCCN3C)nc3c2CCN(c2c(O)cccc2F)C3)C[C@@H]1C ; C=CC(=O)N1[C@H](C)CN(c2c(C#N)c(OC[C@@H]3CCCN3C)nc3c2CCN(c2c(C)cccc2C(F)(F)F)C3)C[C@@H]1C ; C=CC(=O)N1[C@H](C)CN(c2c(C#N)c(OC[C@@H]3CCCN3C)nc3c2CCN(c2c(Cl)c(F)cc4[nH]ncc24)C3)C[C@@H]1C]

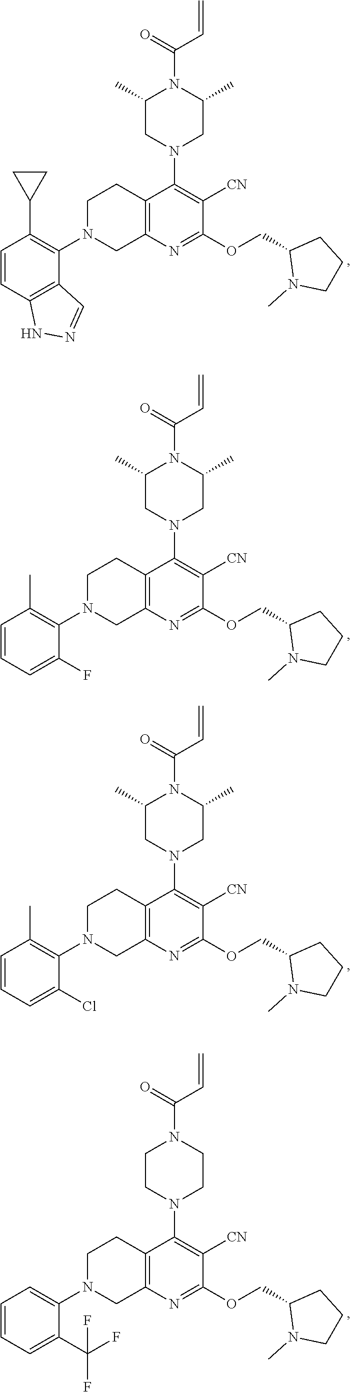 [SMILES: C=CC(=O)N1[C@H](C)CN(c2c(C#N)c(OC[C@@H]3CCCN3C)nc3c2CCN(c2c(C4CC4)ccc4[nH]ncc24)C3)C[C@@H]1C ; C=CC(=O)N1[C@H](C)CN(c2c(C#N)c(OC[C@@H]3CCCN3C)nc3c2CCN(c2c(C)cccc2F)C3)C[C@@H]1C ; C=CC(=O)N1[C@H](C)CN(c2c(C#N)c(OC[C@@H]3CCCN3C)nc3c2CCN(c2c(C)cccc2Cl)C3)C[C@@H]1C ; C=CC(=O)N1CCN(c2c(C#N)c(OC[C@@H]3CCCN3C)nc3c2CCN(c2ccccc2C(F)(F)F)C3)CC1]

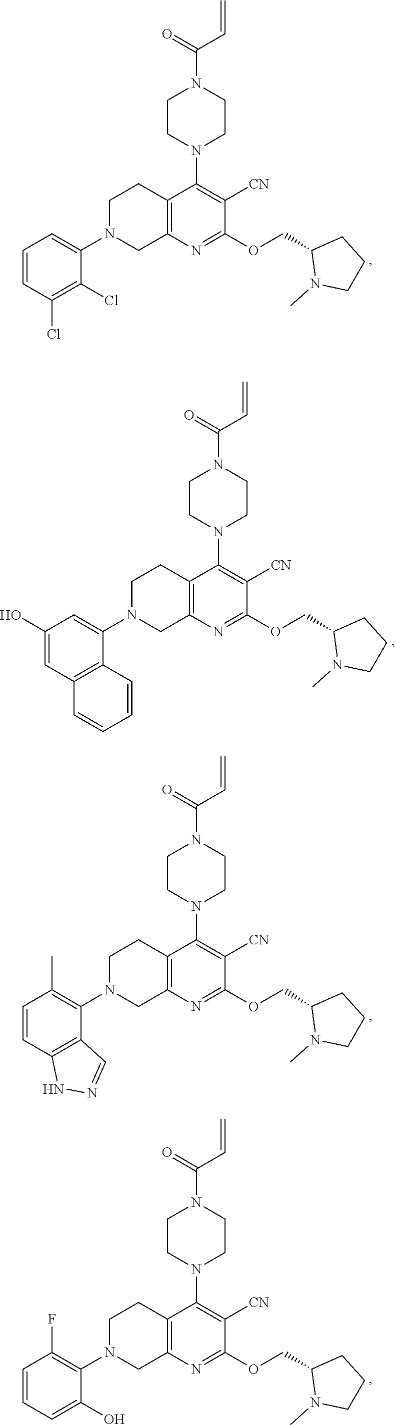 [SMILES: C=CC(=O)N1CCN(c2c(C#N)c(OC[C@@H]3CCCN3C)nc3c2CCN(c2cccc(Cl)c2Cl)C3)CC1 ; C=CC(=O)N1CCN(c2c(C#N)c(OC[C@@H]3CCCN3C)nc3c2CCN(c2cc(O)cc4ccccc24)C3)CC1 ; C=CC(=O)N1CCN(c2c(C#N)c(OC[C@@H]3CCCN3C)nc3c2CCN(c2c(C)ccc4[nH]ncc24)C3)CC1 ; C=CC(=O)N1CCN(c2c(C#N)c(OC[C@@H]3CCCN3C)nc3c2CCN(c2c(O)cccc2F)C3)CC1]

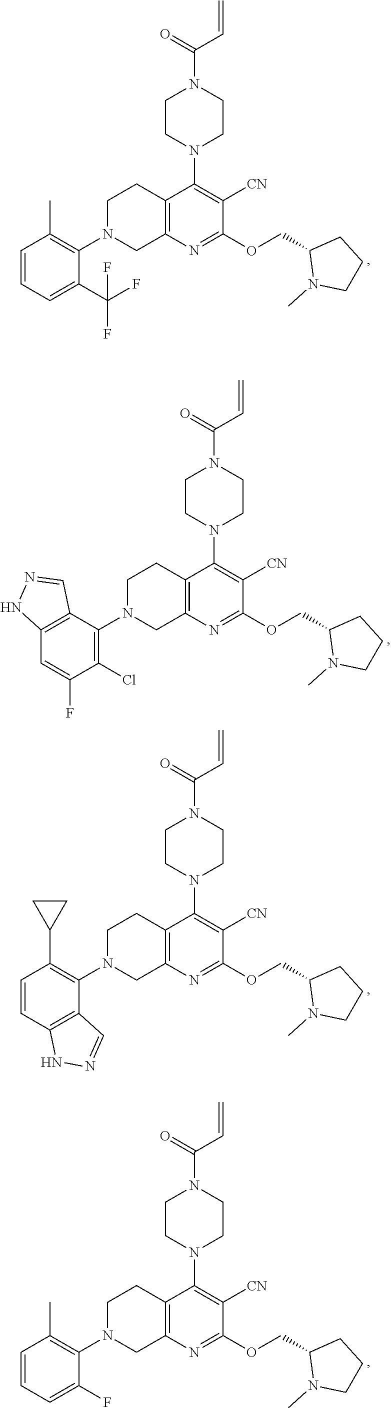 [SMILES: C=CC(=O)N1CCN(c2c(C#N)c(OC[C@@H]3CCCN3C)nc3c2CCN(c2c(C)cccc2C(F)(F)F)C3)CC1 ; C=CC(=O)N1CCN(c2c(C#N)c(OC[C@@H]3CCCN3C)nc3c2CCN(c2c(Cl)c(F)cc4[nH]ncc24)C3)CC1 ; C=CC(=O)N1CCN(c2c(C#N)c(OC[C@@H]3CCCN3C)nc3c2CCN(c2c(C4CC4)ccc4[nH]ncc24)C3)CC1 ; C=CC(=O)N1CCN(c2c(C#N)c(OC[C@@H]3CCCN3C)nc3c2CCN(c2c(C)cccc2F)C3)CC1]

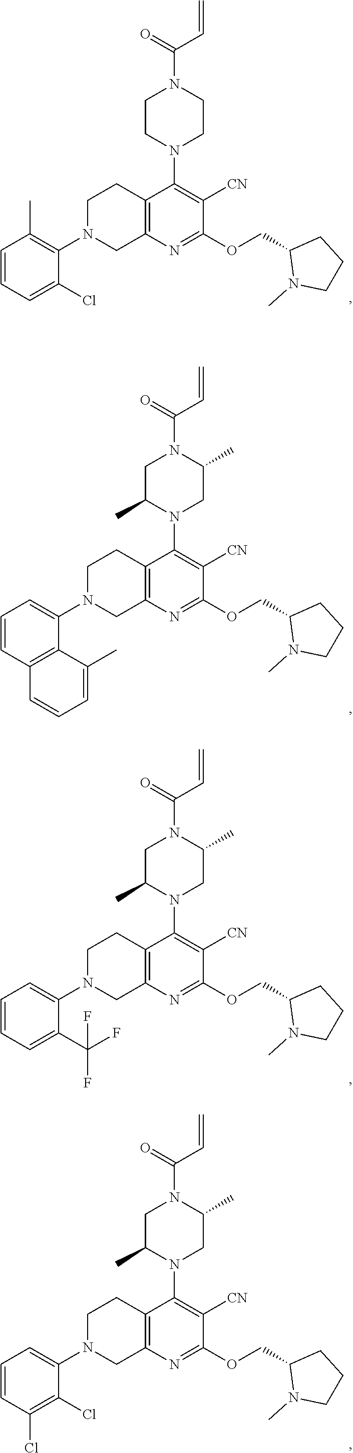 [SMILES: C=CC(=O)N1CCN(c2c(C#N)c(OC[C@@H]3CCCN3C)nc3c2CCN(c2c(C)cccc2Cl)C3)CC1 ; C=CC(=O)N1C[C@H](C)N(c2c(C#N)c(OC[C@@H]3CCCN3C)nc3c2CCN(c2cccc4cccc(C)c24)C3)C[C@H]1C ; C=CC(=O)N1C[C@H](C)N(c2c(C#N)c(OC[C@@H]3CCCN3C)nc3c2CCN(c2ccccc2C(F)(F)F)C3)C[C@H]1C ; C=CC(=O)N1C[C@H](C)N(c2c(C#N)c(OC[C@@H]3CCCN3C)nc3c2CCN(c2cccc(Cl)c2Cl)C3)C[C@H]1C]

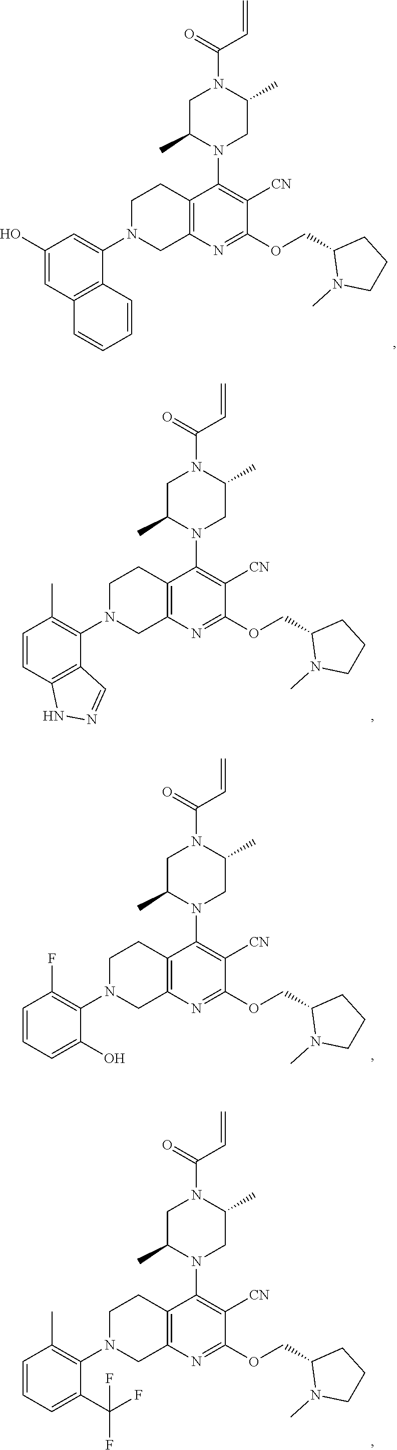 [SMILES: C=CC(=O)N1C[C@H](C)N(c2c(C#N)c(OC[C@@H]3CCCN3C)nc3c2CCN(c2cc(O)cc4ccccc24)C3)C[C@H]1C ; C=CC(=O)N1C[C@H](C)N(c2c(C#N)c(OC[C@@H]3CCCN3C)nc3c2CCN(c2c(C)ccc4[nH]ncc24)C3)C[C@H]1C ; C=CC(=O)N1C[C@H](C)N(c2c(C#N)c(OC[C@@H]3CCCN3C)nc3c2CCN(c2c(O)cccc2F)C3)C[C@H]1C ; C=CC(=O)N1C[C@H](C)N(c2c(C#N)c(OC[C@@H]3CCCN3C)nc3c2CCN(c2c(C)cccc2C(F)(F)F)C3)C[C@H]1C]

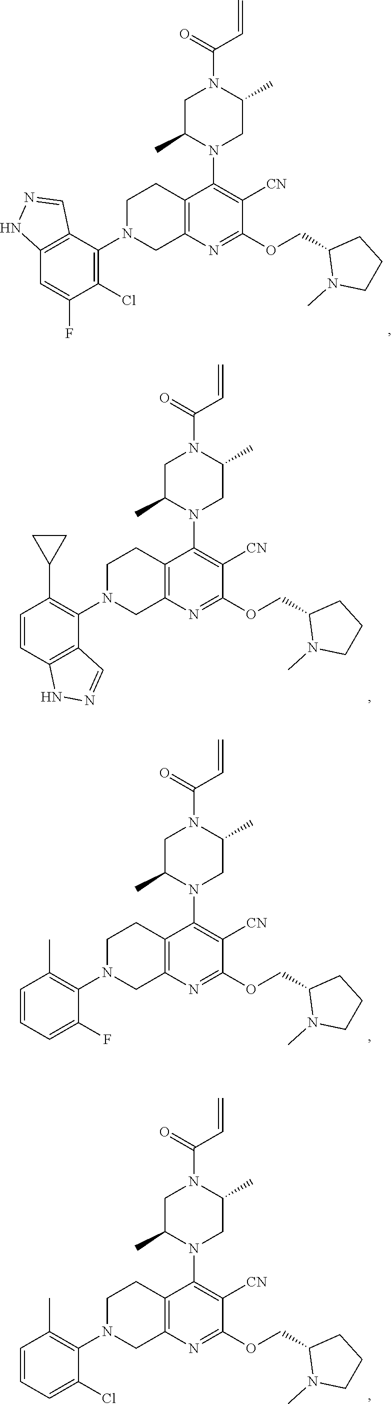 [SMILES: C=CC(=O)N1C[C@H](C)N(c2c(C#N)c(OC[C@@H]3CCCN3C)nc3c2CCN(c2c(Cl)c(F)cc4[nH]ncc24)C3)C[C@H]1C ; C=CC(=O)N1C[C@H](C)N(c2c(C#N)c(OC[C@@H]3CCCN3C)nc3c2CCN(c2c(C4CC4)ccc4[nH]ncc24)C3)C[C@H]1C ; C=CC(=O)N1C[C@H](C)N(c2c(C#N)c(OC[C@@H]3CCCN3C)nc3c2CCN(c2c(C)cccc2F)C3)C[C@H]1C ; C=CC(=O)N1C[C@H](C)N(c2c(C#N)c(OC[C@@H]3CCCN3C)nc3c2CCN(c2c(C)cccc2Cl)C3)C[C@H]1C]

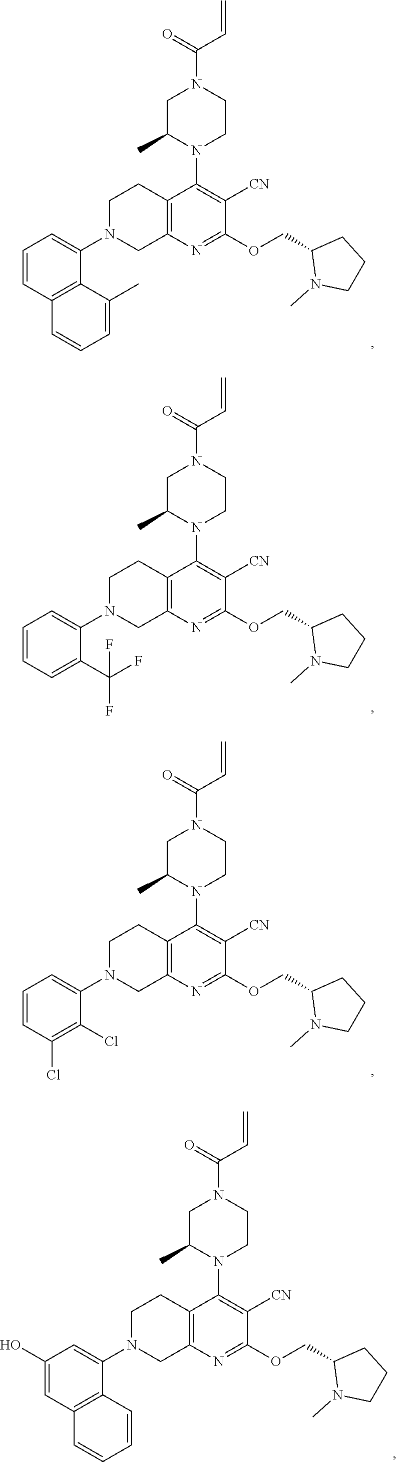 [SMILES: C=CC(=O)N1CCN(c2c(C#N)c(OC[C@@H]3CCCN3C)nc3c2CCN(c2cccc4cccc(C)c24)C3)[C@@H](C)C1 ; C=CC(=O)N1CCN(c2c(C#N)c(OC[C@@H]3CCCN3C)nc3c2CCN(c2ccccc2C(F)(F)F)C3)[C@@H](C)C1 ; C=CC(=O)N1CCN(c2c(C#N)c(OC[C@@H]3CCCN3C)nc3c2CCN(c2cccc(Cl)c2Cl)C3)[C@@H](C)C1 ; C=CC(=O)N1CCN(c2c(C#N)c(OC[C@@H]3CCCN3C)nc3c2CCN(c2cc(O)cc4ccccc24)C3)[C@@H](C)C1]

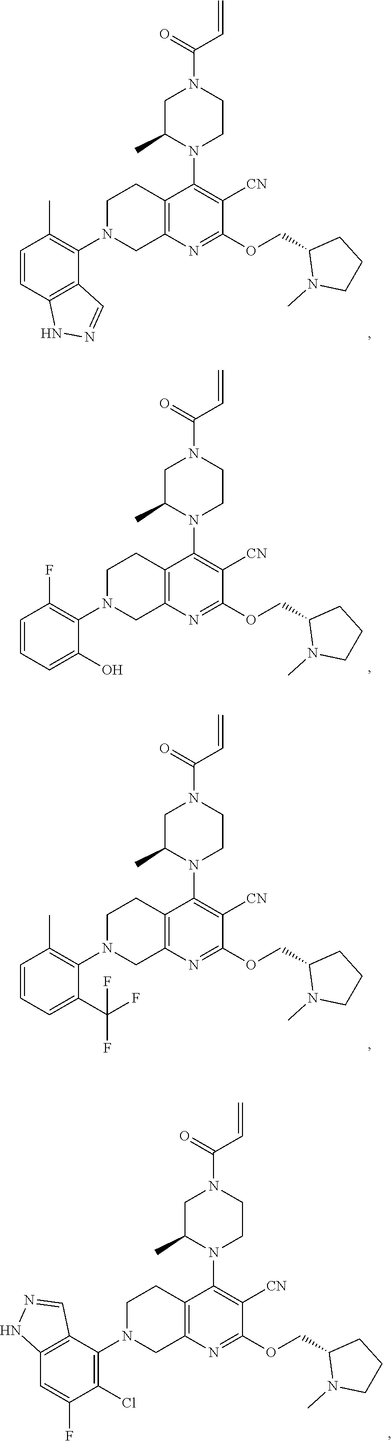 [SMILES: C=CC(=O)N1CCN(c2c(C#N)c(OC[C@@H]3CCCN3C)nc3c2CCN(c2c(C)ccc4[nH]ncc24)C3)[C@@H](C)C1 ; C=CC(=O)N1CCN(c2c(C#N)c(OC[C@@H]3CCCN3C)nc3c2CCN(c2c(O)cccc2F)C3)[C@@H](C)C1 ; C=CC(=O)N1CCN(c2c(C#N)c(OC[C@@H]3CCCN3C)nc3c2CCN(c2c(C)cccc2C(F)(F)F)C3)[C@@H](C)C1 ; C=CC(=O)N1CCN(c2c(C#N)c(OC[C@@H]3CCCN3C)nc3c2CCN(c2c(Cl)c(F)cc4[nH]ncc24)C3)[C@@H](C)C1]

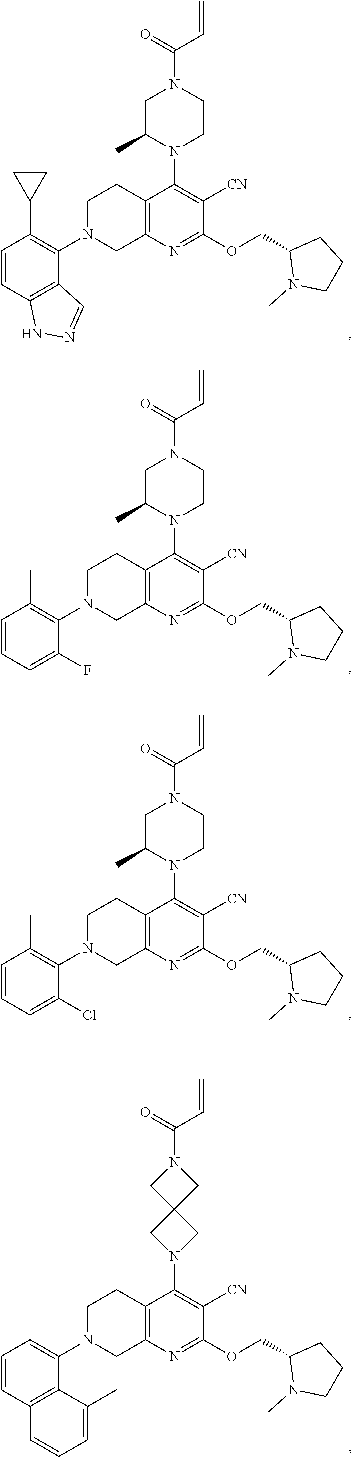 [SMILES: C=CC(=O)N1CCN(c2c(C#N)c(OC[C@@H]3CCCN3C)nc3c2CCN(c2c(C4CC4)ccc4[nH]ncc24)C3)[C@@H](C)C1 ; C=CC(=O)N1CCN(c2c(C#N)c(OC[C@@H]3CCCN3C)nc3c2CCN(c2c(C)cccc2F)C3)[C@@H](C)C1 ; C=CC(=O)N1CCN(c2c(C#N)c(OC[C@@H]3CCCN3C)nc3c2CCN(c2c(C)cccc2Cl)C3)[C@@H](C)C1 ; C=CC(=O)N1CC2(C1)CN(c1c(C#N)c(OC[C@@H]3CCCN3C)nc3c1CCN(c1cccc4cccc(C)c14)C3)C2]

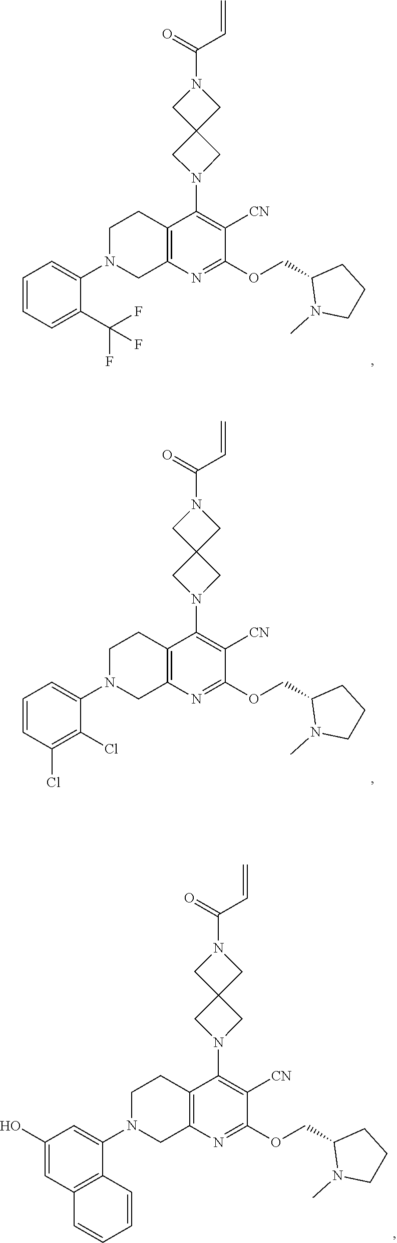 [SMILES: C=CC(=O)N1CC2(C1)CN(c1c(C#N)c(OC[C@@H]3CCCN3C)nc3c1CCN(c1ccccc1C(F)(F)F)C3)C2 ; C=CC(=O)N1CC2(C1)CN(c1c(C#N)c(OC[C@@H]3CCCN3C)nc3c1CCN(c1cccc(Cl)c1Cl)C3)C2 ; C=CC(=O)N1CC2(C1)CN(c1c(C#N)c(OC[C@@H]3CCCN3C)nc3c1CCN(c1cc(O)cc4ccccc14)C3)C2]

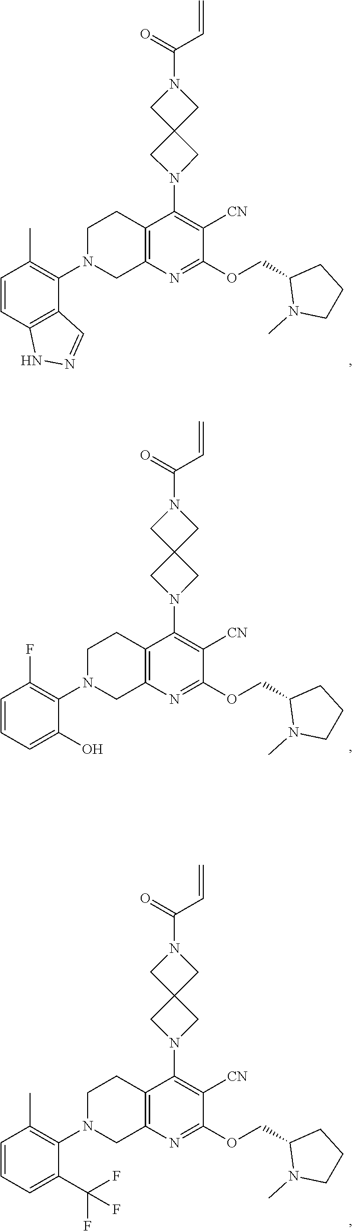 [SMILES: C=CC(=O)N1CC2(C1)CN(c1c(C#N)c(OC[C@@H]3CCCN3C)nc3c1CCN(c1c(C)ccc4[nH]ncc14)C3)C2 ; C=CC(=O)N1CC2(C1)CN(c1c(C#N)c(OC[C@@H]3CCCN3C)nc3c1CCN(c1c(O)cccc1F)C3)C2 ; C=CC(=O)N1CC2(C1)CN(c1c(C#N)c(OC[C@@H]3CCCN3C)nc3c1CCN(c1c(C)cccc1C(F)(F)F)C3)C2]

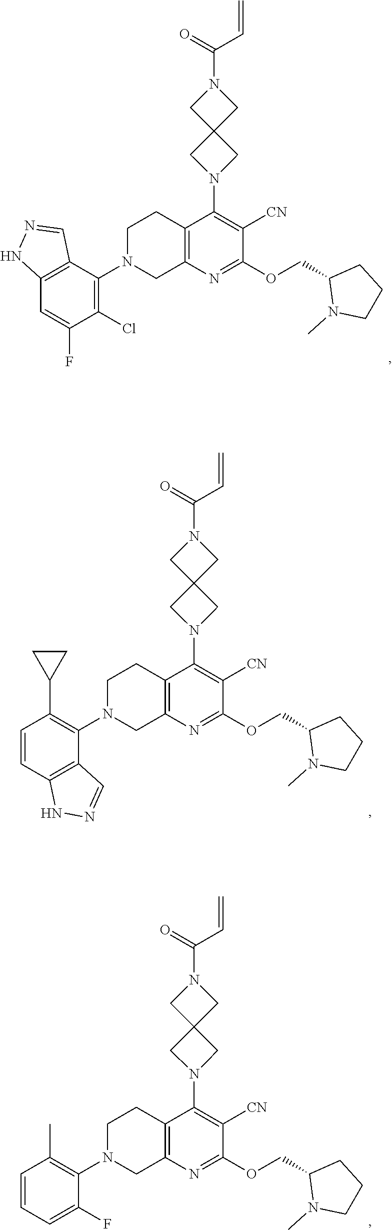 [SMILES: C=CC(=O)N1CC2(C1)CN(c1c(C#N)c(OC[C@@H]3CCCN3C)nc3c1CCN(c1c(Cl)c(F)cc4[nH]ncc14)C3)C2 ; C=CC(=O)N1CC2(C1)CN(c1c(C#N)c(OC[C@@H]3CCCN3C)nc3c1CCN(c1c(C4CC4)ccc4[nH]ncc14)C3)C2 ; C=CC(=O)N1CC2(C1)CN(c1c(C#N)c(OC[C@@H]3CCCN3C)nc3c1CCN(c1c(C)cccc1F)C3)C2]

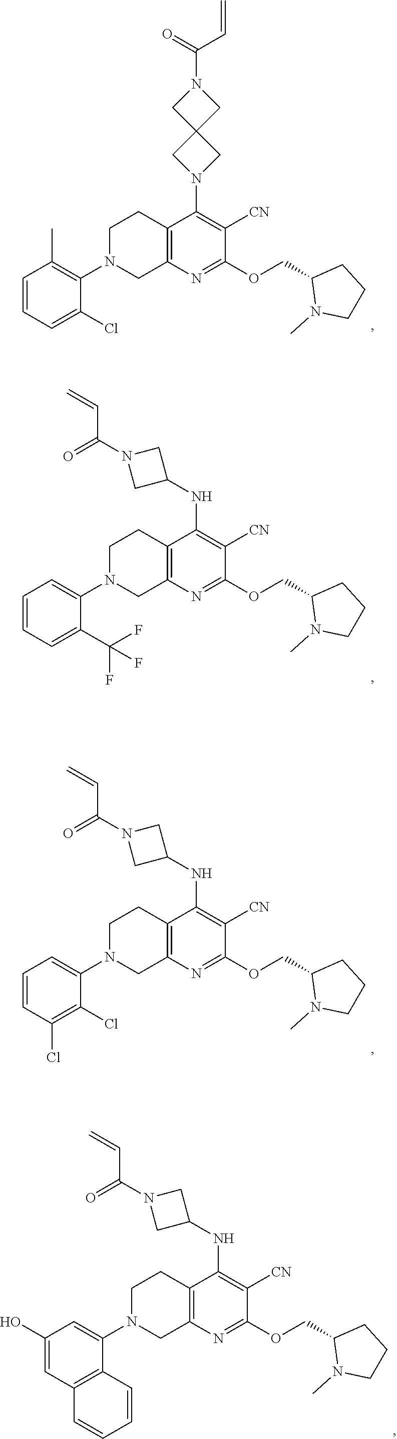 [SMILES: C=CC(=O)N1CC2(C1)CN(c1c(C#N)c(OC[C@@H]3CCCN3C)nc3c1CCN(c1c(C)cccc1Cl)C3)C2 ; C=CC(=O)N1CC(Nc2c(C#N)c(OC[C@@H]3CCCN3C)nc3c2CCN(c2ccccc2C(F)(F)F)C3)C1 ; C=CC(=O)N1CC(Nc2c(C#N)c(OC[C@@H]3CCCN3C)nc3c2CCN(c2cccc(Cl)c2Cl)C3)C1 ; C=CC(=O)N1CC(Nc2c(C#N)c(OC[C@@H]3CCCN3C)nc3c2CCN(c2cc(O)cc4ccccc24)C3)C1]

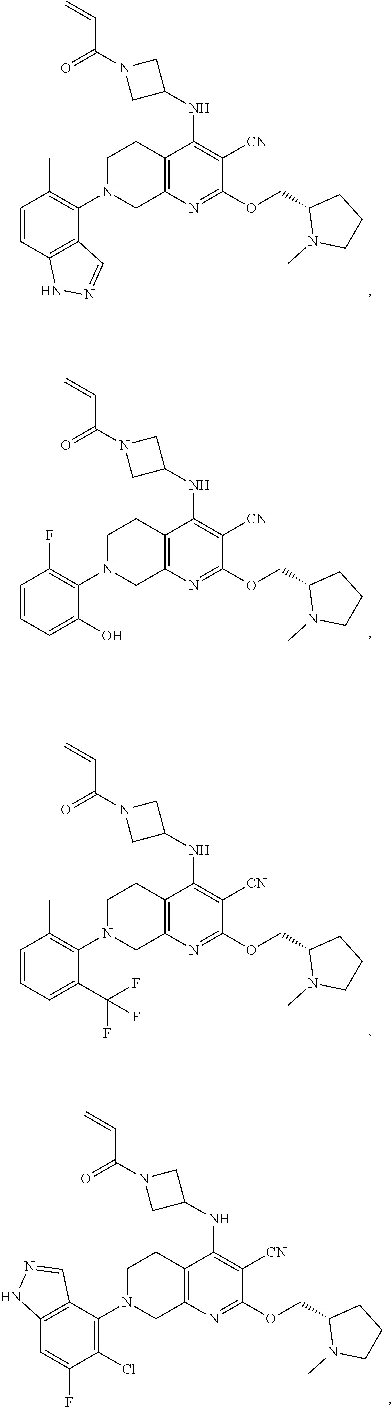 [SMILES: C=CC(=O)N1CC(Nc2c(C#N)c(OC[C@@H]3CCCN3C)nc3c2CCN(c2c(C)ccc4[nH]ncc24)C3)C1 ; C=CC(=O)N1CC(Nc2c(C#N)c(OC[C@@H]3CCCN3C)nc3c2CCN(c2c(O)cccc2F)C3)C1 ; C=CC(=O)N1CC(Nc2c(C#N)c(OC[C@@H]3CCCN3C)nc3c2CCN(c2c(C)cccc2C(F)(F)F)C3)C1 ; C=CC(=O)N1CC(Nc2c(C#N)c(OC[C@@H]3CCCN3C)nc3c2CCN(c2c(Cl)c(F)cc4[nH]ncc24)C3)C1]

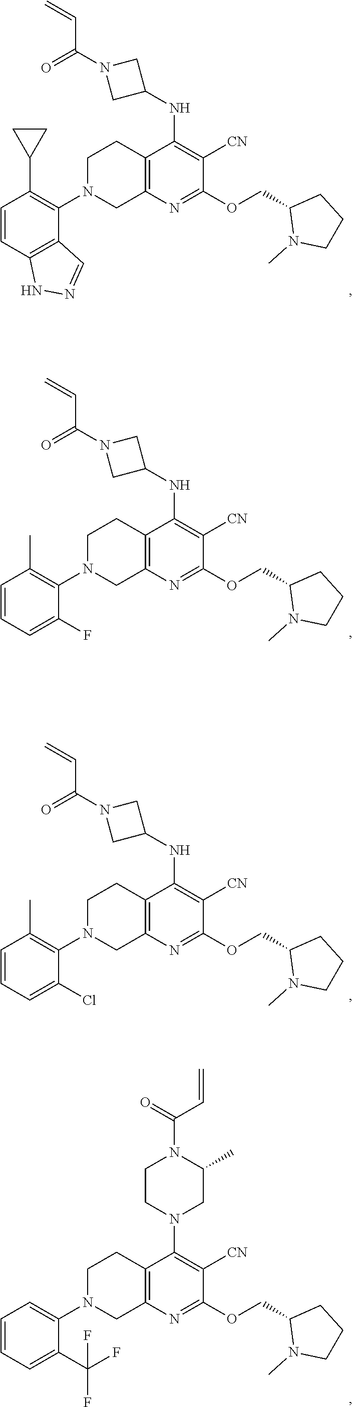 [SMILES: C=CC(=O)N1CC(Nc2c(C#N)c(OC[C@@H]3CCCN3C)nc3c2CCN(c2c(C4CC4)ccc4[nH]ncc24)C3)C1 ; C=CC(=O)N1CC(Nc2c(C#N)c(OC[C@@H]3CCCN3C)nc3c2CCN(c2c(C)cccc2F)C3)C1 ; C=CC(=O)N1CC(Nc2c(C#N)c(OC[C@@H]3CCCN3C)nc3c2CCN(c2c(C)cccc2Cl)C3)C1 ; C=CC(=O)N1CCN(c2c(C#N)c(OC[C@@H]3CCCN3C)nc3c2CCN(c2ccccc2C(F)(F)F)C3)C[C@H]1C]

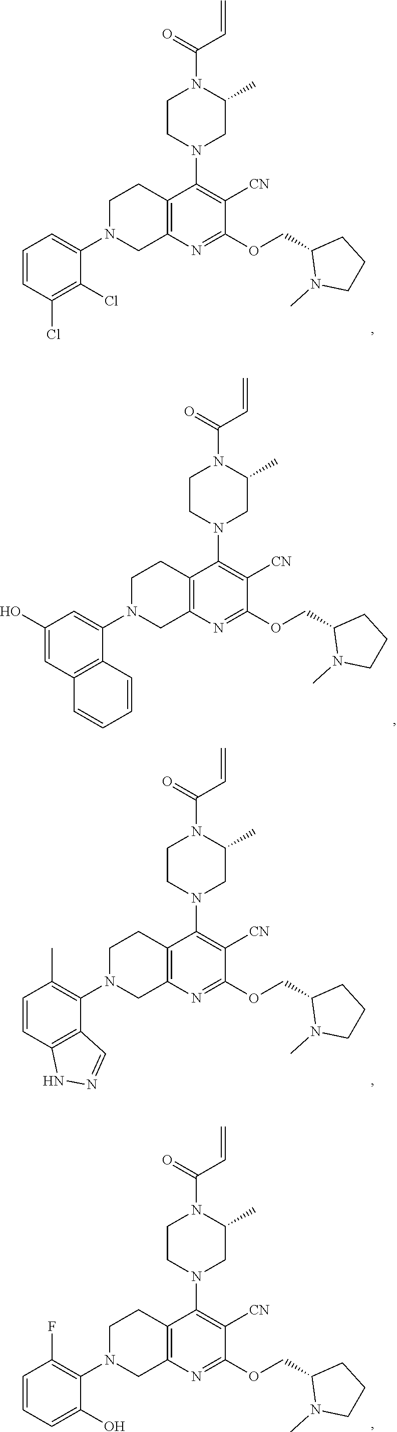 [SMILES: C=CC(=O)N1CCN(c2c(C#N)c(OC[C@@H]3CCCN3C)nc3c2CCN(c2cccc(Cl)c2Cl)C3)C[C@H]1C ; C=CC(=O)N1CCN(c2c(C#N)c(OC[C@@H]3CCCN3C)nc3c2CCN(c2cc(O)cc4ccccc24)C3)C[C@H]1C ; C=CC(=O)N1CCN(c2c(C#N)c(OC[C@@H]3CCCN3C)nc3c2CCN(c2c(C)ccc4[nH]ncc24)C3)C[C@H]1C ; C=CC(=O)N1CCN(c2c(C#N)c(OC[C@@H]3CCCN3C)nc3c2CCN(c2c(O)cccc2F)C3)C[C@H]1C]

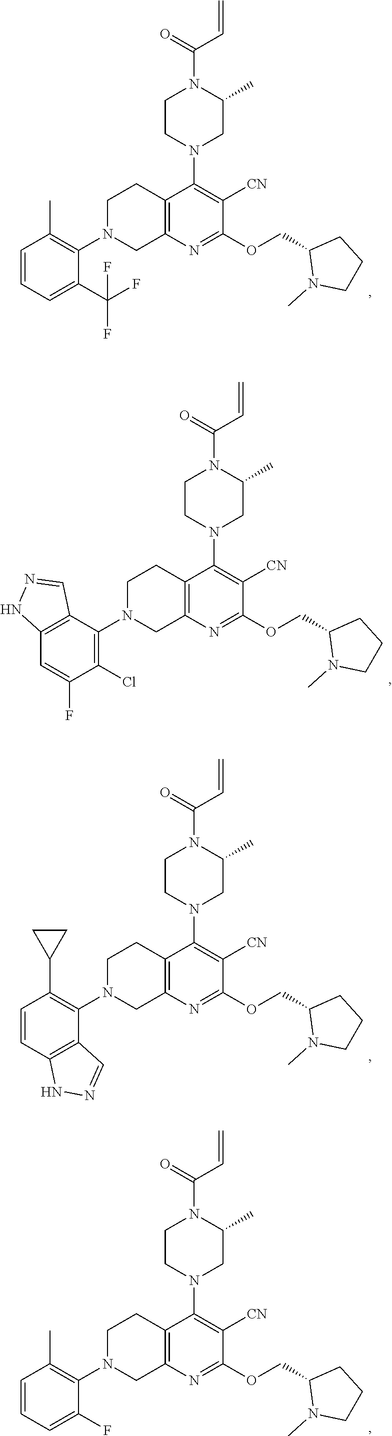 [SMILES: C=CC(=O)N1CCN(c2c(C#N)c(OC[C@@H]3CCCN3C)nc3c2CCN(c2c(C)cccc2C(F)(F)F)C3)C[C@H]1C ; C=CC(=O)N1CCN(c2c(C#N)c(OC[C@@H]3CCCN3C)nc3c2CCN(c2c(Cl)c(F)cc4[nH]ncc24)C3)C[C@H]1C ; C=CC(=O)N1CCN(c2c(C#N)c(OC[C@@H]3CCCN3C)nc3c2CCN(c2c(C4CC4)ccc4[nH]ncc24)C3)C[C@H]1C ; C=CC(=O)N1CCN(c2c(C#N)c(OC[C@@H]3CCCN3C)nc3c2CCN(c2c(C)cccc2F)C3)C[C@H]1C]

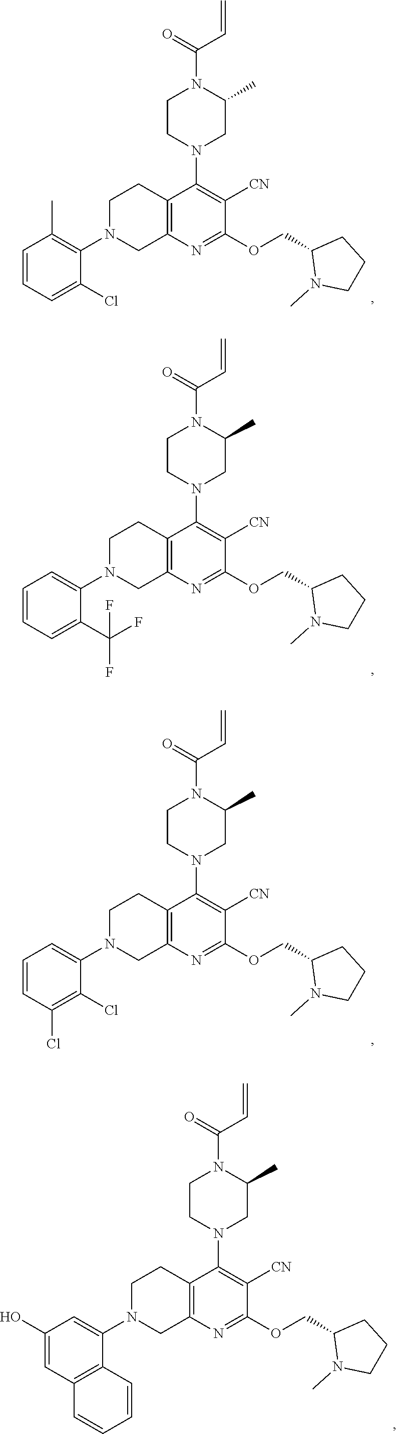 [SMILES: C=CC(=O)N1CCN(c2c(C#N)c(OC[C@@H]3CCCN3C)nc3c2CCN(c2c(C)cccc2Cl)C3)C[C@H]1C ; C=CC(=O)N1CCN(c2c(C#N)c(OC[C@@H]3CCCN3C)nc3c2CCN(c2ccccc2C(F)(F)F)C3)C[C@@H]1C ; C=CC(=O)N1CCN(c2c(C#N)c(OC[C@@H]3CCCN3C)nc3c2CCN(c2cccc(Cl)c2Cl)C3)C[C@@H]1C ; C=CC(=O)N1CCN(c2c(C#N)c(OC[C@@H]3CCCN3C)nc3c2CCN(c2cc(O)cc4ccccc24)C3)C[C@@H]1C]

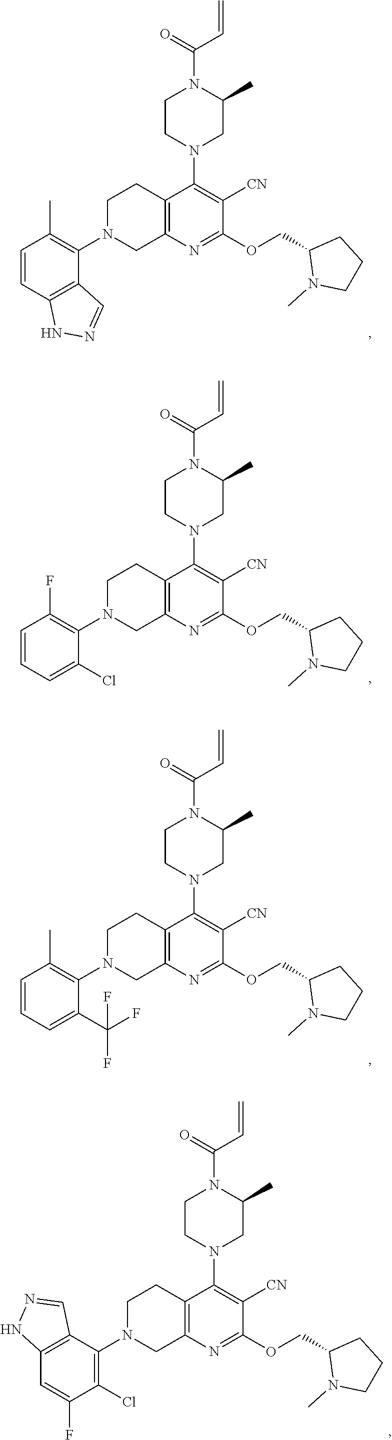 [SMILES: C=CC(=O)N1CCN(c2c(C#N)c(OC[C@@H]3CCCN3C)nc3c2CCN(c2c(C)ccc4[nH]ncc24)C3)C[C@@H]1C ; C=CC(=O)N1CCN(c2c(C#N)c(OC[C@@H]3CCCN3C)nc3c2CCN(c2c(F)cccc2Cl)C3)C[C@@H]1C ; C=CC(=O)N1CCN(c2c(C#N)c(OC[C@@H]3CCCN3C)nc3c2CCN(c2c(C)cccc2C(F)(F)F)C3)C[C@@H]1C ; C=CC(=O)N1CCN(c2c(C#N)c(OC[C@@H]3CCCN3C)nc3c2CCN(c2c(Cl)c(F)cc4[nH]ncc24)C3)C[C@@H]1C]

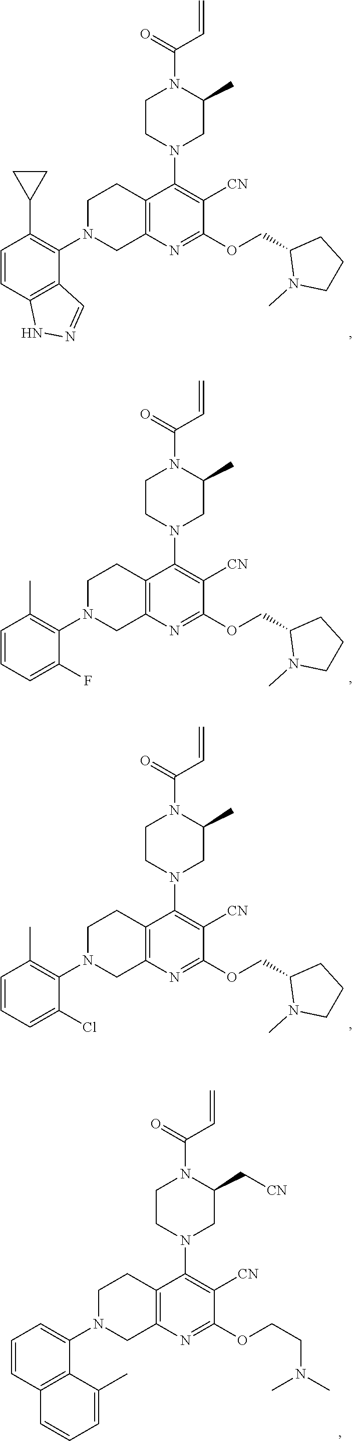 [SMILES: C=CC(=O)N1CCN(c2c(C#N)c(OC[C@@H]3CCCN3C)nc3c2CCN(c2c(C4CC4)ccc4[nH]ncc24)C3)C[C@@H]1C ; C=CC(=O)N1CCN(c2c(C#N)c(OC[C@@H]3CCCN3C)nc3c2CCN(c2c(C)cccc2F)C3)C[C@@H]1C ; C=CC(=O)N1CCN(c2c(C#N)c(OC[C@@H]3CCCN3C)nc3c2CCN(c2c(C)cccc2Cl)C3)C[C@@H]1C ; C=CC(=O)N1CCN(c2c(C#N)c(OCCN(C)C)nc3c2CCN(c2cccc4cccc(C)c24)C3)C[C@@H]1CC#N]

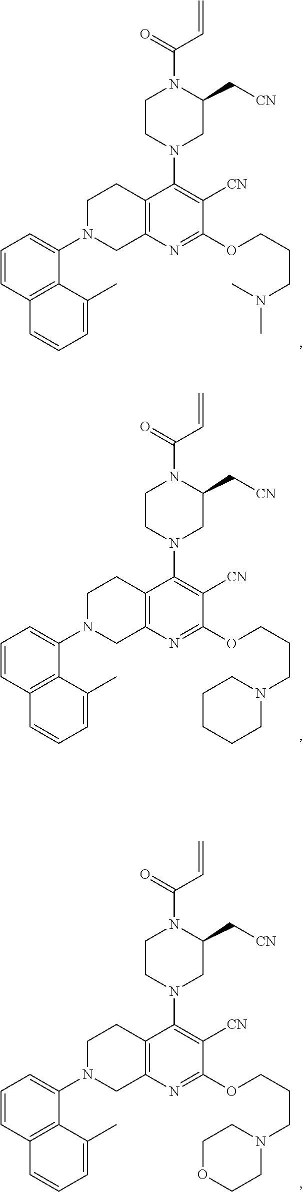 [SMILES: C=CC(=O)N1CCN(c2c(C#N)c(OCCCN(C)C)nc3c2CCN(c2cccc4cccc(C)c24)C3)C[C@@H]1CC#N ; C=CC(=O)N1CCN(c2c(C#N)c(OCCCN3CCCCC3)nc3c2CCN(c2cccc4cccc(C)c24)C3)C[C@@H]1CC#N ; C=CC(=O)N1CCN(c2c(C#N)c(OCCCN3CCOCC3)nc3c2CCN(c2cccc4cccc(C)c24)C3)C[C@@H]1CC#N]

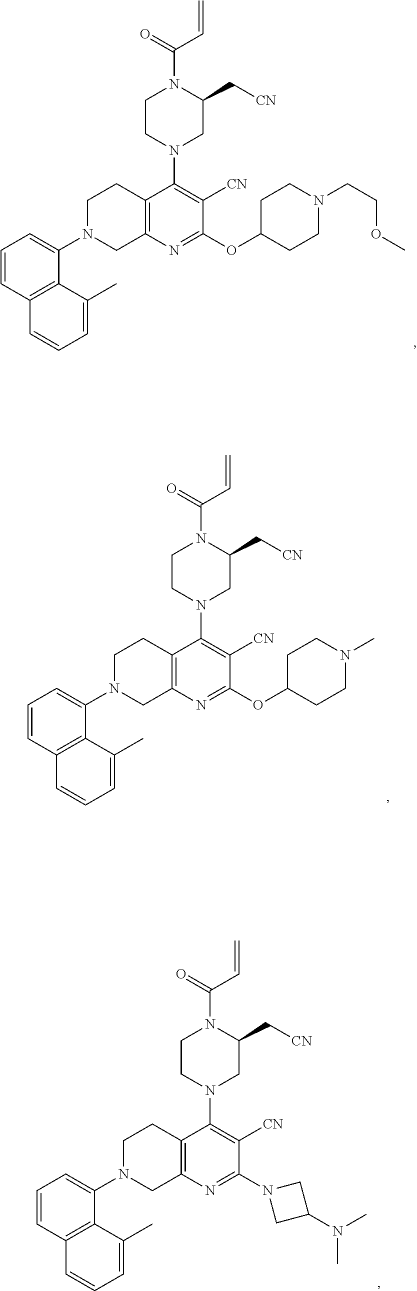 [SMILES: C=CC(=O)N1CCN(c2c(C#N)c(OC3CCN(CCOC)CC3)nc3c2CCN(c2cccc4cccc(C)c24)C3)C[C@@H]1CC#N ; C=CC(=O)N1CCN(c2c(C#N)c(OC3CCN(C)CC3)nc3c2CCN(c2cccc4cccc(C)c24)C3)C[C@@H]1CC#N ; C=CC(=O)N1CCN(c2c(C#N)c(N3CC(N(C)C)C3)nc3c2CCN(c2cccc4cccc(C)c24)C3)C[C@@H]1CC#N]

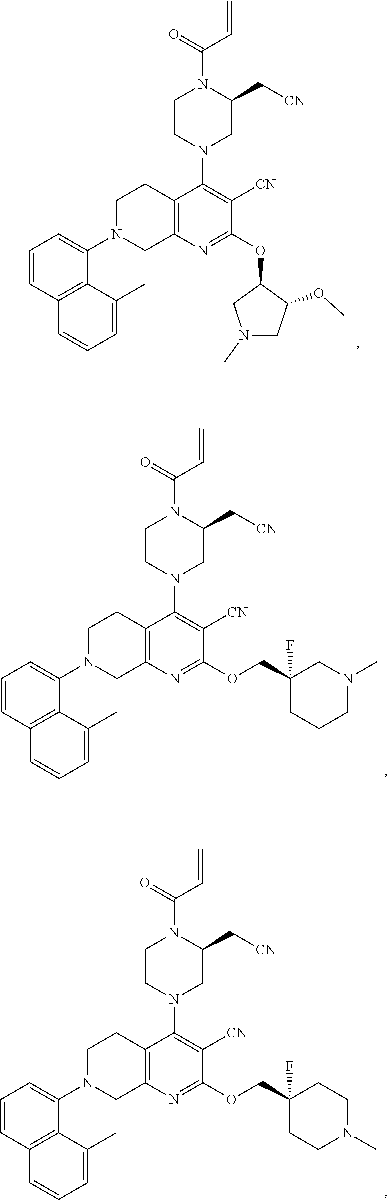

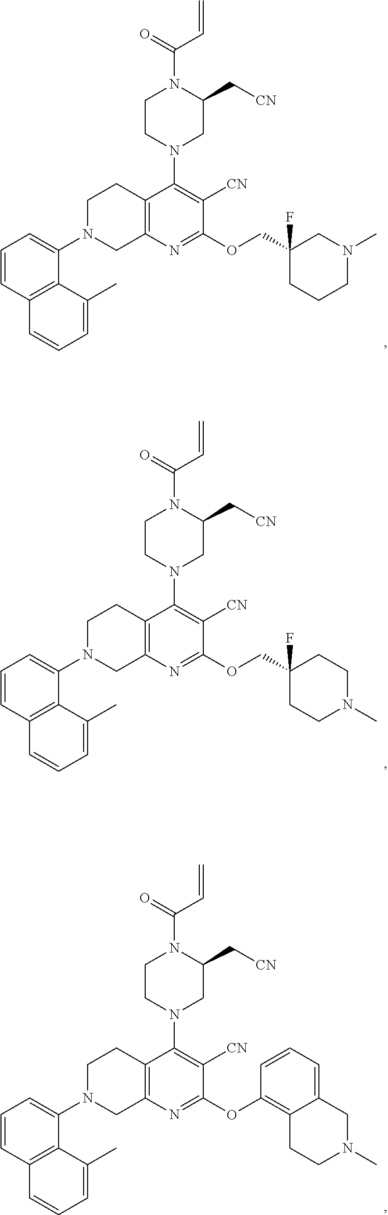

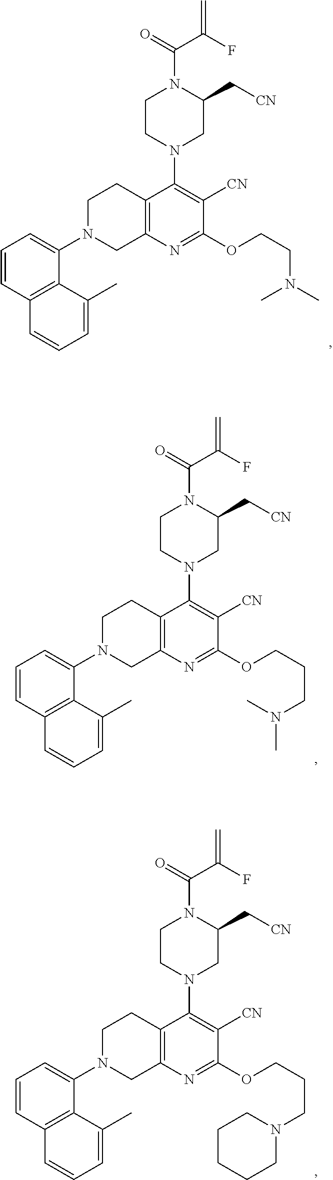 [SMILES: C=C(F)C(=O)N1CCN(c2c(C#N)c(OCCN(C)C)nc3c2CCN(c2cccc4cccc(C)c24)C3)C[C@@H]1CC#N ; C=C(F)C(=O)N1CCN(c2c(C#N)c(OCCCN(C)C)nc3c2CCN(c2cccc4cccc(C)c24)C3)C[C@@H]1CC#N ; C=C(F)C(=O)N1CCN(c2c(C#N)c(OCCCN3CCCCC3)nc3c2CCN(c2cccc4cccc(C)c24)C3)C[C@@H]1CC#N]

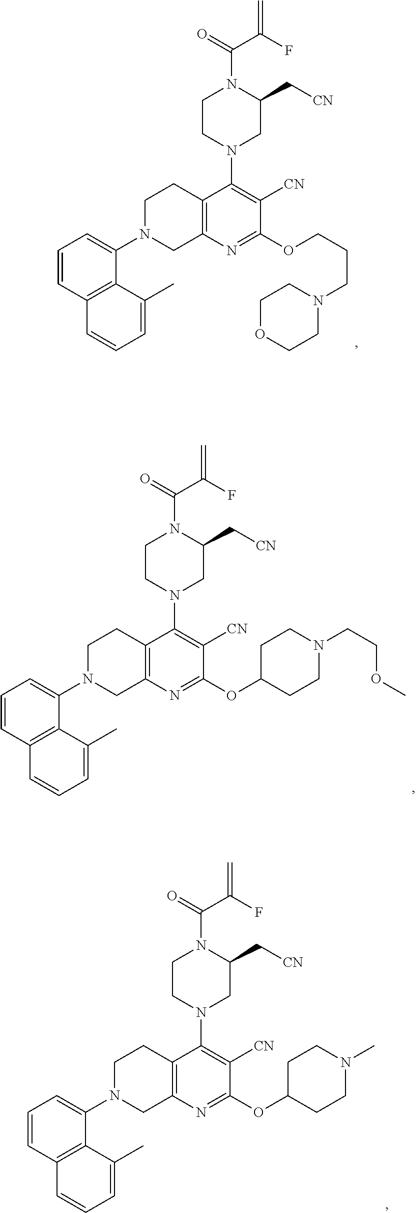 [SMILES: C=C(F)C(=O)N1CCN(c2c(C#N)c(OCCCN3CCOCC3)nc3c2CCN(c2cccc4cccc(C)c24)C3)C[C@@H]1CC#N ; C=C(F)C(=O)N1CCN(c2c(C#N)c(OC3CCN(CCOC)CC3)nc3c2CCN(c2cccc4cccc(C)c24)C3)C[C@@H]1CC#N ; C=C(F)C(=O)N1CCN(c2c(C#N)c(OC3CCN(C)CC3)nc3c2CCN(c2cccc4cccc(C)c24)C3)C[C@@H]1CC#N]

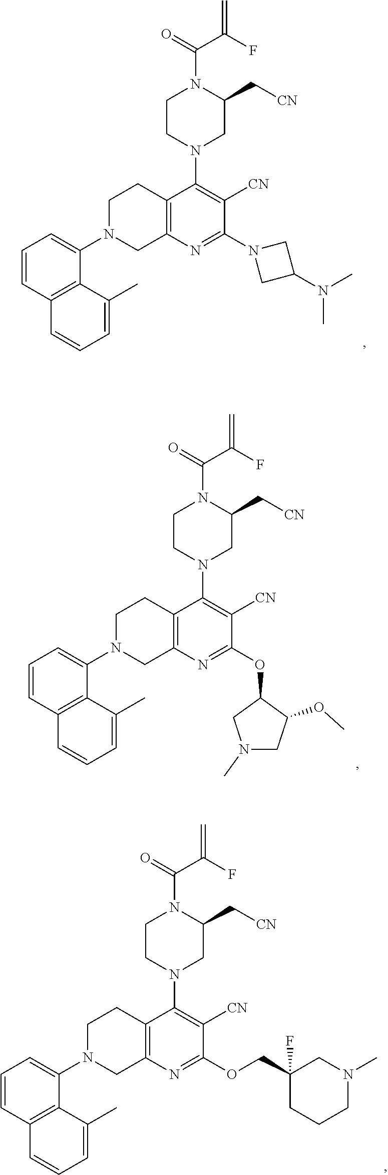 [SMILES: C=C(F)C(=O)N1CCN(c2c(C#N)c(N3CC(N(C)C)C3)nc3c2CCN(c2cccc4cccc(C)c24)C3)C[C@@H]1CC#N ; C=C(F)C(=O)N1CCN(c2c(C#N)c(O[C@@H]3CN(C)C[C@H]3OC)nc3c2CCN(c2cccc4cccc(C)c24)C3)C[C@@H]1CC#N ; C=C(F)C(=O)N1CCN(c2c(C#N)c(OC[C@]3(F)CCCN(C)C3)nc3c2CCN(c2cccc4cccc(C)c24)C3)C[C@@H]1CC#N]

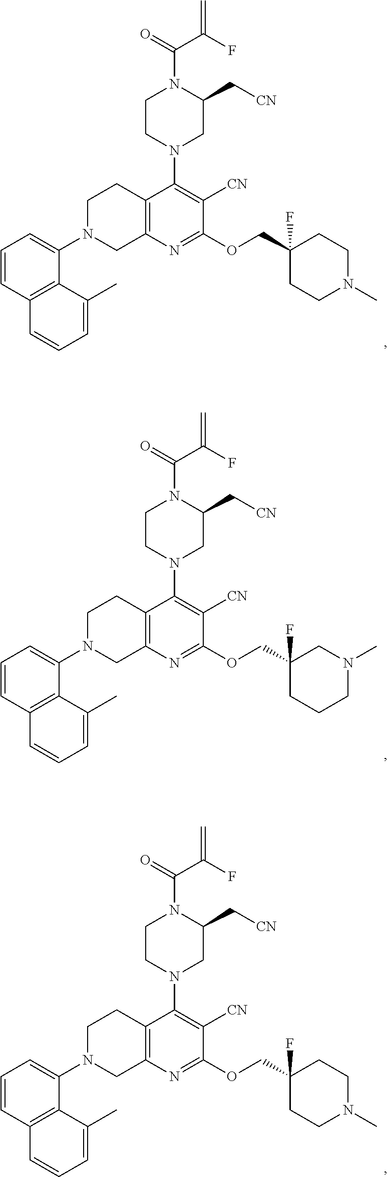

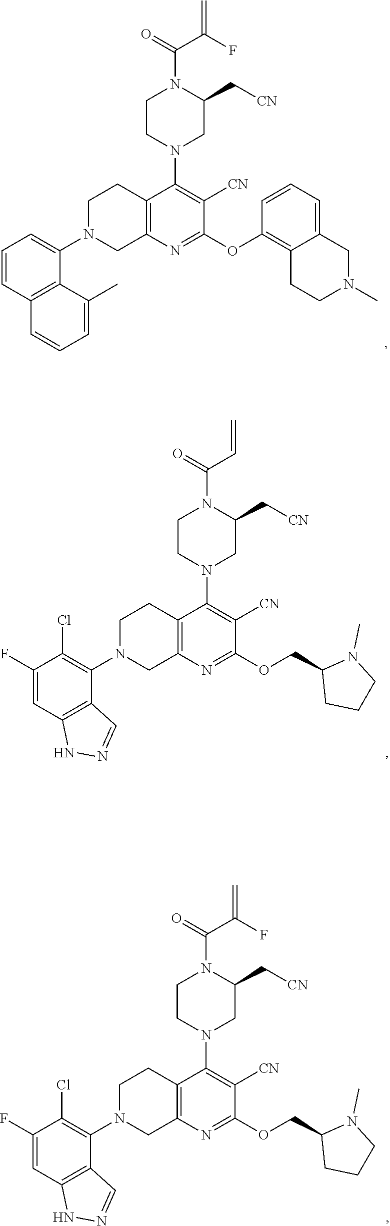 [SMILES: C=C(F)C(=O)N1CCN(c2c(C#N)c(Oc3cccc4c3CCN(C)C4)nc3c2CCN(c2cccc4cccc(C)c24)C3)C[C@@H]1CC#N ; C=CC(=O)N1CCN(c2c(C#N)c(OC[C@@H]3CCCN3C)nc3c2CCN(c2c(Cl)c(F)cc4[nH]ncc24)C3)C[C@@H]1CC#N ; C=C(F)C(=O)N1CCN(c2c(C#N)c(OC[C@@H]3CCCN3C)nc3c2CCN(c2c(Cl)c(F)cc4[nH]ncc24)C3)C[C@@H]1CC#N]

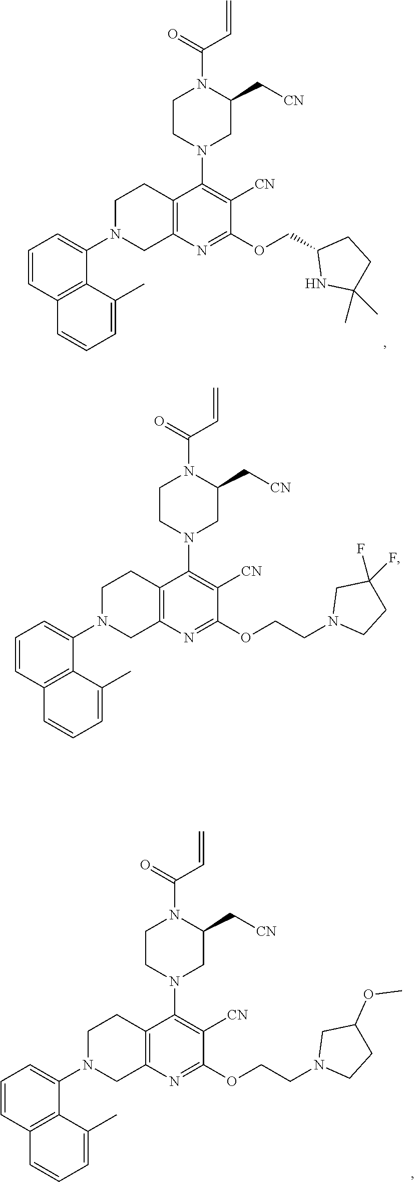 [SMILES: C=CC(=O)N1CCN(c2c(C#N)c(OC[C@@H]3CCC(C)(C)N3)nc3c2CCN(c2cccc4cccc(C)c24)C3)C[C@@H]1CC#N ; C=CC(=O)N1CCN(c2c(C#N)c(OCCN3CCC(F)(F)C3)nc3c2CCN(c2cccc4cccc(C)c24)C3)C[C@@H]1CC#N ; C=CC(=O)N1CCN(c2c(C#N)c(OCCN3CCC(OC)C3)nc3c2CCN(c2cccc4cccc(C)c24)C3)C[C@@H]1CC#N]

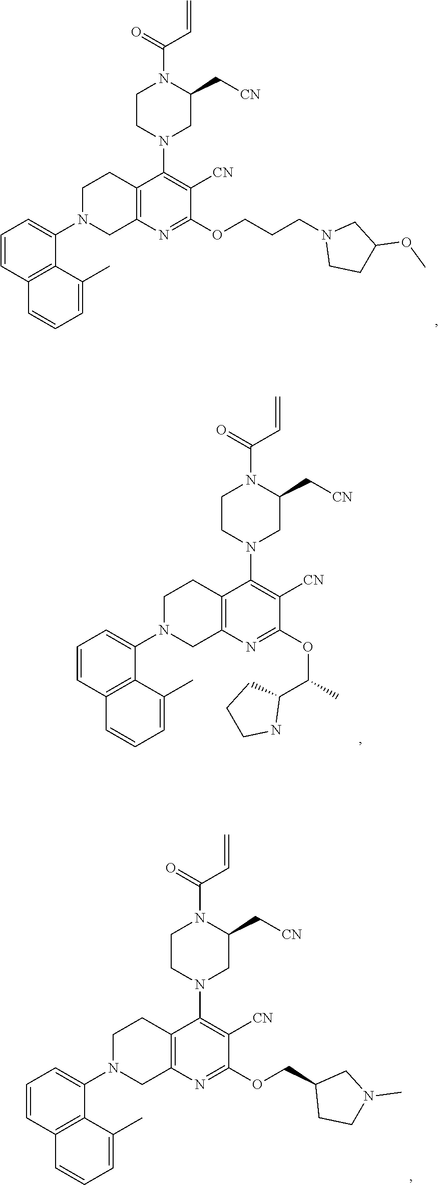 [SMILES: C=CC(=O)N1CCN(c2c(C#N)c(OCCCN3CCC(OC)C3)nc3c2CCN(c2cccc4cccc(C)c24)C3)C[C@@H]1CC#N ; C=CC(=O)N1CCN(c2c(C#N)c(O[C@H](C)[C@H]3CCCN3)nc3c2CCN(c2cccc4cccc(C)c24)C3)C[C@@H]1CC#N ; C=CC(=O)N1CCN(c2c(C#N)c(OC[C@@H]3CCN(C)C3)nc3c2CCN(c2cccc4cccc(C)c24)C3)C[C@@H]1CC#N]

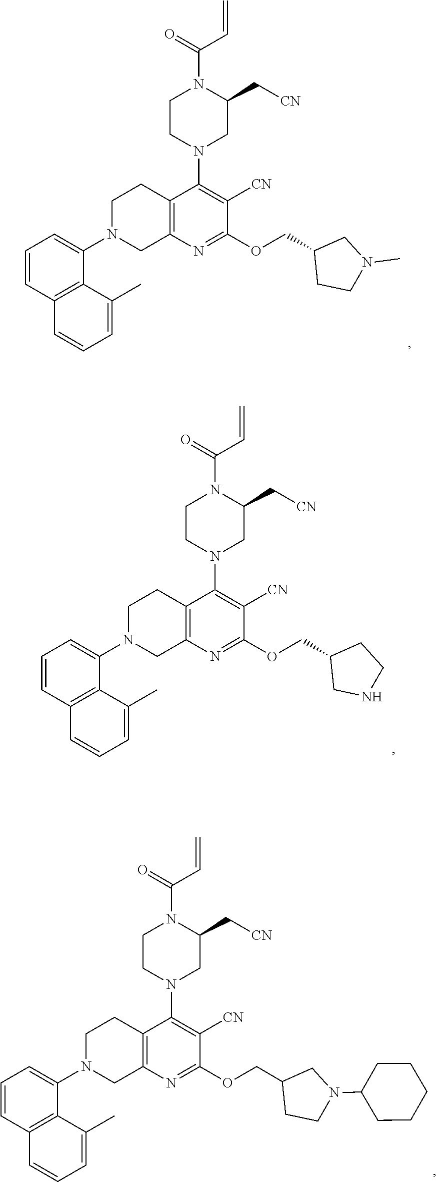 [SMILES: C=CC(=O)N1CCN(c2c(C#N)c(OC[C@H]3CCN(C)C3)nc3c2CCN(c2cccc4cccc(C)c24)C3)C[C@@H]1CC#N ; C=CC(=O)N1CCN(c2c(C#N)c(OC[C@@H]3CCNC3)nc3c2CCN(c2cccc4cccc(C)c24)C3)C[C@@H]1CC#N ; C=CC(=O)N1CCN(c2c(C#N)c(OCC3CCN(C4CCCCC4)C3)nc3c2CCN(c2cccc4cccc(C)c24)C3)C[C@@H]1CC#N]

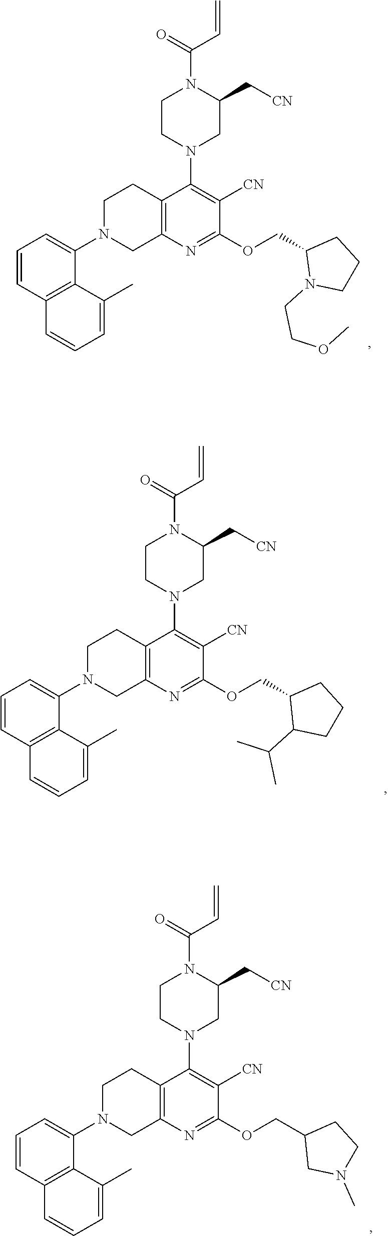 [SMILES: C=CC(=O)N1CCN(c2c(C#N)c(OC[C@@H]3CCCN3CCOC)nc3c2CCN(c2cccc4cccc(C)c24)C3)C[C@@H]1CC#N ; C=CC(=O)N1CCN(c2c(C#N)c(OC[C@@H]3CCCC3C(C)C)nc3c2CCN(c2cccc4cccc(C)c24)C3)C[C@@H]1CC#N ; C=CC(=O)N1CCN(c2c(C#N)c(OCC3CCN(C)C3)nc3c2CCN(c2cccc4cccc(C)c24)C3)C[C@@H]1CC#N]

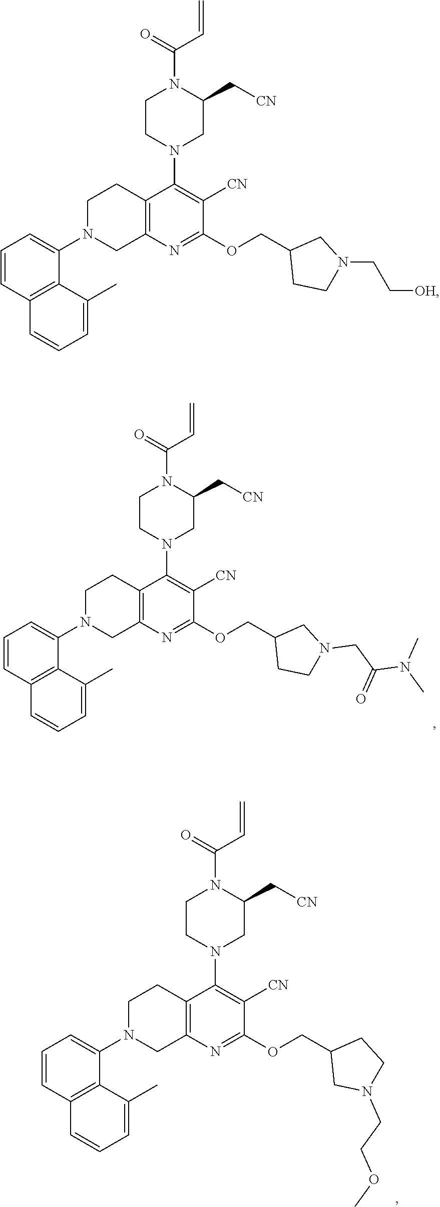 [SMILES: C=CC(=O)N1CCN(c2c(C#N)c(OCC3CCN(CCO)C3)nc3c2CCN(c2cccc4cccc(C)c24)C3)C[C@@H]1CC#N ; C=CC(=O)N1CCN(c2c(C#N)c(OCC3CCN(CC(=O)N(C)C)C3)nc3c2CCN(c2cccc4cccc(C)c24)C3)C[C@@H]1CC#N ; C=CC(=O)N1CCN(c2c(C#N)c(OCC3CCN(CCOC)C3)nc3c2CCN(c2cccc4cccc(C)c24)C3)C[C@@H]1CC#N]

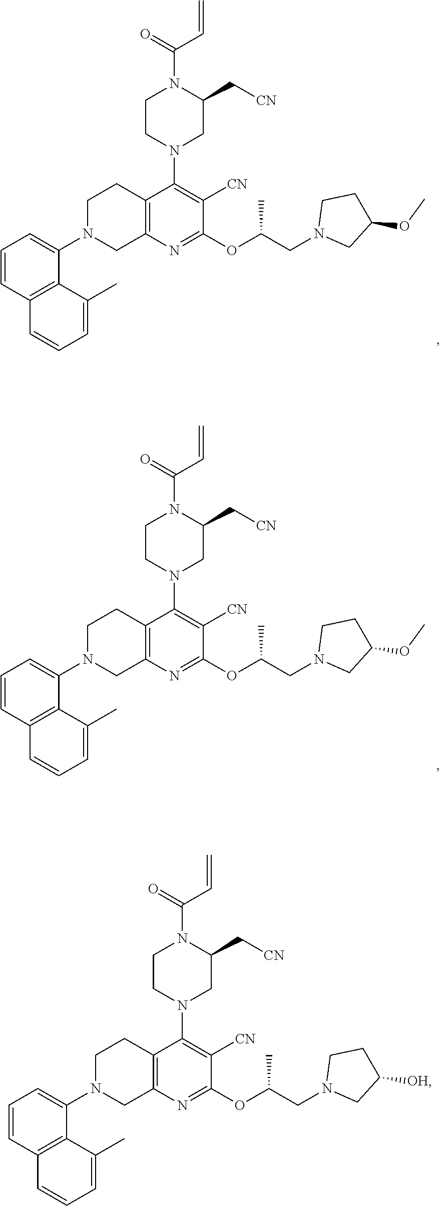 [SMILES: C=CC(=O)N1CCN(c2c(C#N)c(O[C@H](C)CN3CC[C@@H](OC)C3)nc3c2CCN(c2cccc4cccc(C)c24)C3)C[C@@H]1CC#N ; C=CC(=O)N1CCN(c2c(C#N)c(O[C@H](C)CN3CC[C@H](OC)C3)nc3c2CCN(c2cccc4cccc(C)c24)C3)C[C@@H]1CC#N ; C=CC(=O)N1CCN(c2c(C#N)c(O[C@H](C)CN3CC[C@H](O)C3)nc3c2CCN(c2cccc4cccc(C)c24)C3)C[C@@H]1CC#N]

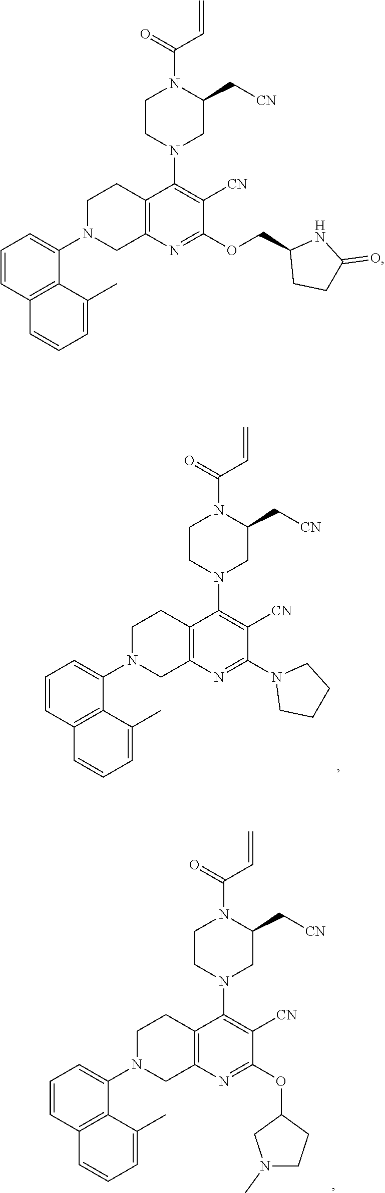 [SMILES: C=CC(=O)N1CCN(c2c(C#N)c(OC[C@@H]3CCC(=O)N3)nc3c2CCN(c2cccc4cccc(C)c24)C3)C[C@@H]1CC#N ; C=CC(=O)N1CCN(c2c(C#N)c(N3CCCC3)nc3c2CCN(c2cccc4cccc(C)c24)C3)C[C@@H]1CC#N ; C=CC(=O)N1CCN(c2c(C#N)c(OC3CCN(C)C3)nc3c2CCN(c2cccc4cccc(C)c24)C3)C[C@@H]1CC#N]

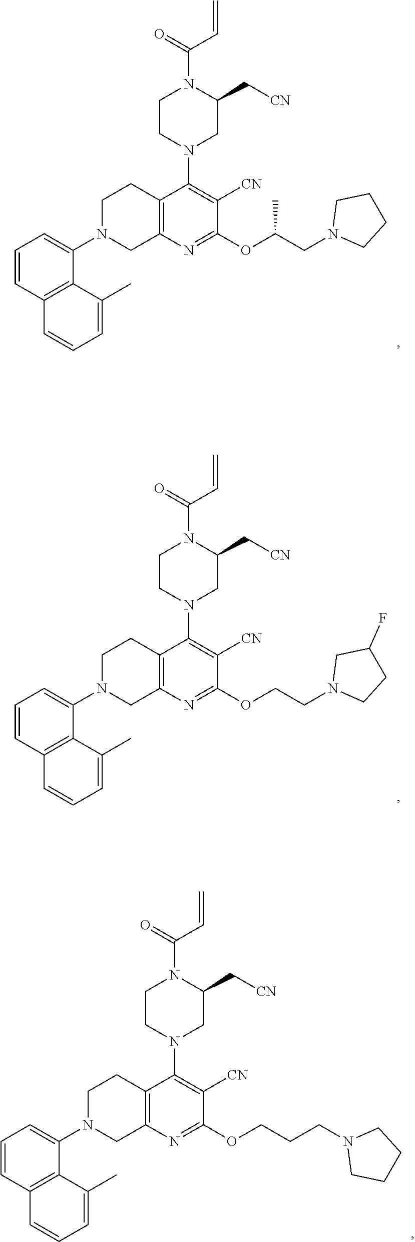 [SMILES: C=CC(=O)N1CCN(c2c(C#N)c(O[C@H](C)CN3CCCC3)nc3c2CCN(c2cccc4cccc(C)c24)C3)C[C@@H]1CC#N ; C=CC(=O)N1CCN(c2c(C#N)c(OCCN3CCC(F)C3)nc3c2CCN(c2cccc4cccc(C)c24)C3)C[C@@H]1CC#N ; C=CC(=O)N1CCN(c2c(C#N)c(OCCCN3CCCC3)nc3c2CCN(c2cccc4cccc(C)c24)C3)C[C@@H]1CC#N]

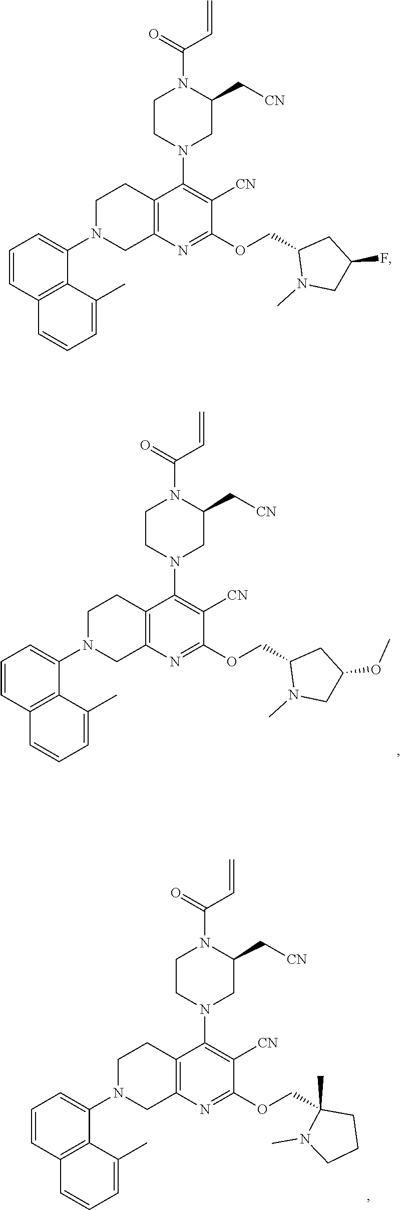 [SMILES: C=CC(=O)N1CCN(c2c(C#N)c(OC[C@@H]3C[C@@H](F)CN3C)nc3c2CCN(c2cccc4cccc(C)c24)C3)C[C@@H]1CC#N ; C=CC(=O)N1CCN(c2c(C#N)c(OC[C@@H]3C[C@H](OC)CN3C)nc3c2CCN(c2cccc4cccc(C)c24)C3)C[C@@H]1CC#N ; C=CC(=O)N1CCN(c2c(C#N)c(OC[C@]3(C)CCCN3C)nc3c2CCN(c2cccc4cccc(C)c24)C3)C[C@@H]1CC#N]

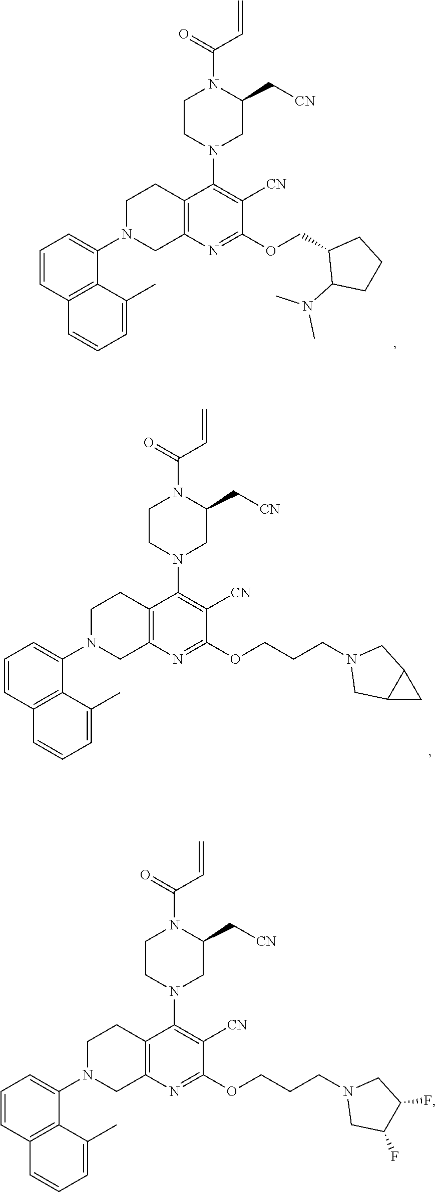 [SMILES: C=CC(=O)N1CCN(c2c(C#N)c(OC[C@@H]3CCCC3N(C)C)nc3c2CCN(c2cccc4cccc(C)c24)C3)C[C@@H]1CC#N ; C=CC(=O)N1CCN(c2c(C#N)c(OCCCN3CC4CC4C3)nc3c2CCN(c2cccc4cccc(C)c24)C3)C[C@@H]1CC#N ; C=CC(=O)N1CCN(c2c(C#N)c(OCCCN3C[C@@H](F)[C@@H](F)C3)nc3c2CCN(c2cccc4cccc(C)c24)C3)C[C@@H]1CC#N]

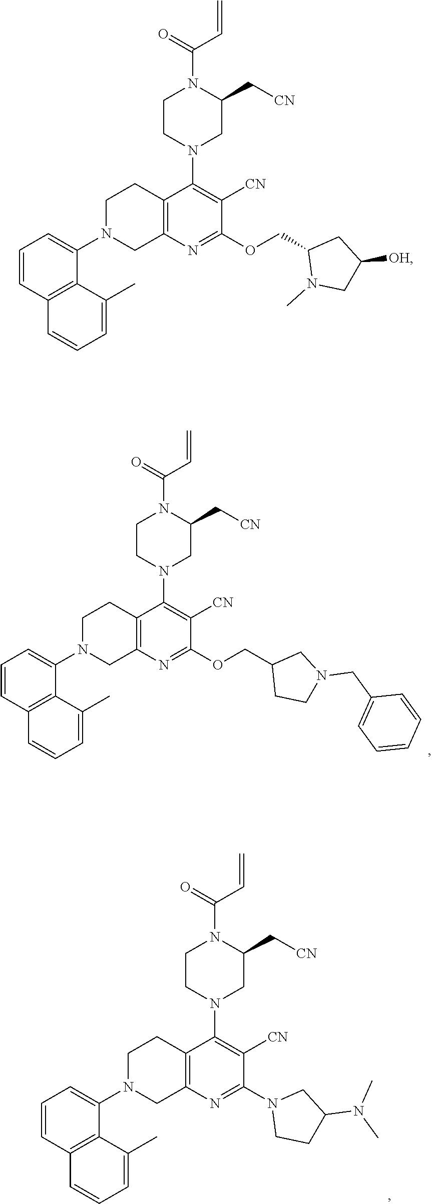 [SMILES: C=CC(=O)N1CCN(c2c(C#N)c(OC[C@@H]3C[C@@H](O)CN3C)nc3c2CCN(c2cccc4cccc(C)c24)C3)C[C@@H]1CC#N ; C=CC(=O)N1CCN(c2c(C#N)c(OCC3CCN(Cc4ccccc4)C3)nc3c2CCN(c2cccc4cccc(C)c24)C3)C[C@@H]1CC#N ; C=CC(=O)N1CCN(c2c(C#N)c(N3CCC(N(C)C)C3)nc3c2CCN(c2cccc4cccc(C)c24)C3)C[C@@H]1CC#N]

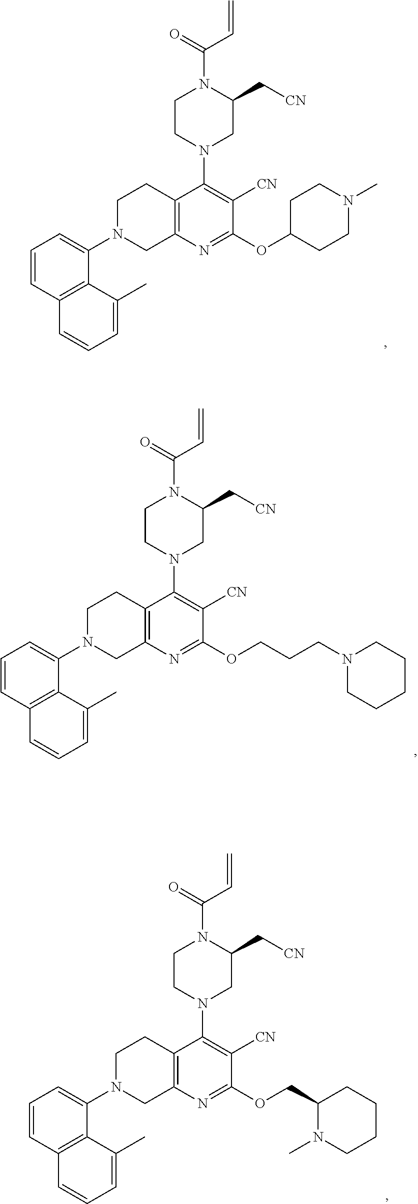 [SMILES: C=CC(=O)N1CCN(c2c(C#N)c(OC3CCN(C)CC3)nc3c2CCN(c2cccc4cccc(C)c24)C3)C[C@@H]1CC#N ; C=CC(=O)N1CCN(c2c(C#N)c(OCCCN3CCCCC3)nc3c2CCN(c2cccc4cccc(C)c24)C3)C[C@@H]1CC#N ; C=CC(=O)N1CCN(c2c(C#N)c(OC[C@H]3CCCCN3C)nc3c2CCN(c2cccc4cccc(C)c24)C3)C[C@@H]1CC#N]

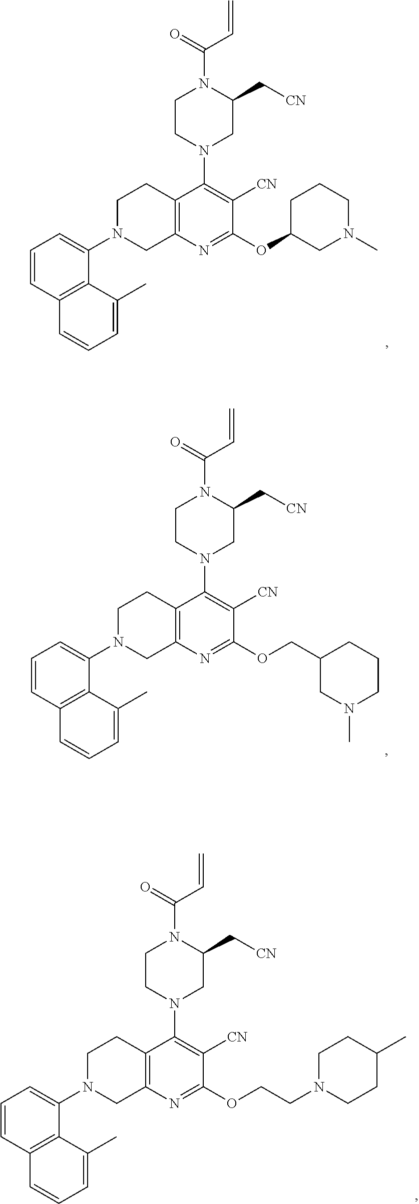 [SMILES: C=CC(=O)N1CCN(c2c(C#N)c(O[C@H]3CCCN(C)C3)nc3c2CCN(c2cccc4cccc(C)c24)C3)C[C@@H]1CC#N ; C=CC(=O)N1CCN(c2c(C#N)c(OCC3CCCN(C)C3)nc3c2CCN(c2cccc4cccc(C)c24)C3)C[C@@H]1CC#N ; C=CC(=O)N1CCN(c2c(C#N)c(OCCN3CCC(C)CC3)nc3c2CCN(c2cccc4cccc(C)c24)C3)C[C@@H]1CC#N]

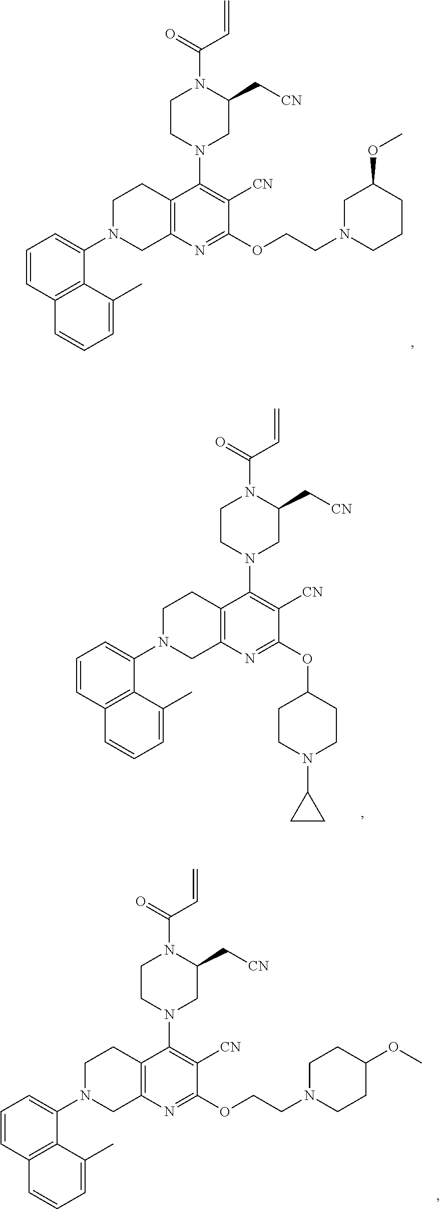 [SMILES: C=CC(=O)N1CCN(c2c(C#N)c(OCCN3CCC[C@H](OC)C3)nc3c2CCN(c2cccc4cccc(C)c24)C3)C[C@@H]1CC#N ; C=CC(=O)N1CCN(c2c(C#N)c(OC3CCN(C4CC4)CC3)nc3c2CCN(c2cccc4cccc(C)c24)C3)C[C@@H]1CC#N ; C=CC(=O)N1CCN(c2c(C#N)c(OCCN3CCC(OC)CC3)nc3c2CCN(c2cccc4cccc(C)c24)C3)C[C@@H]1CC#N]

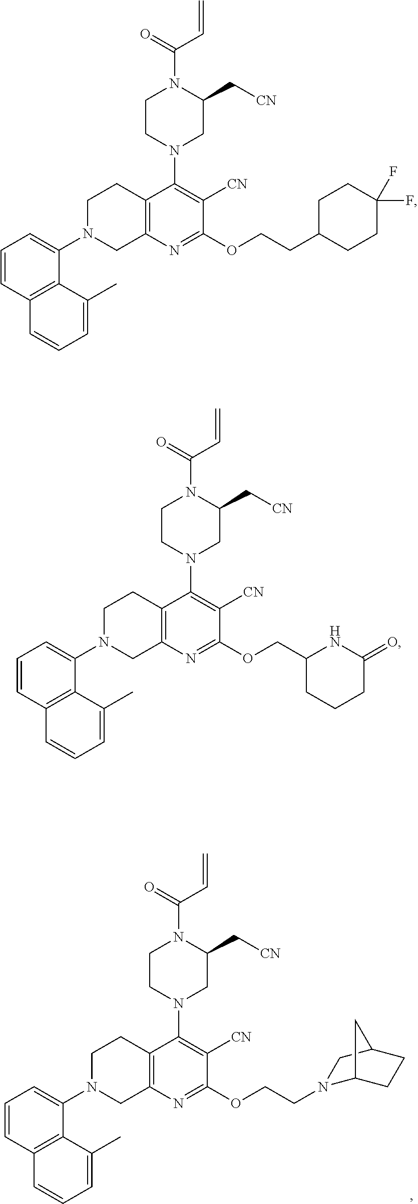 [SMILES: C=CC(=O)N1CCN(c2c(C#N)c(OCCC3CCC(F)(F)CC3)nc3c2CCN(c2cccc4cccc(C)c24)C3)C[C@@H]1CC#N ; C=CC(=O)N1CCN(c2c(C#N)c(OCC3CCCC(=O)N3)nc3c2CCN(c2cccc4cccc(C)c24)C3)C[C@@H]1CC#N ; C=CC(=O)N1CCN(c2c(C#N)c(OCCN3CC4CCC3C4)nc3c2CCN(c2cccc4cccc(C)c24)C3)C[C@@H]1CC#N]

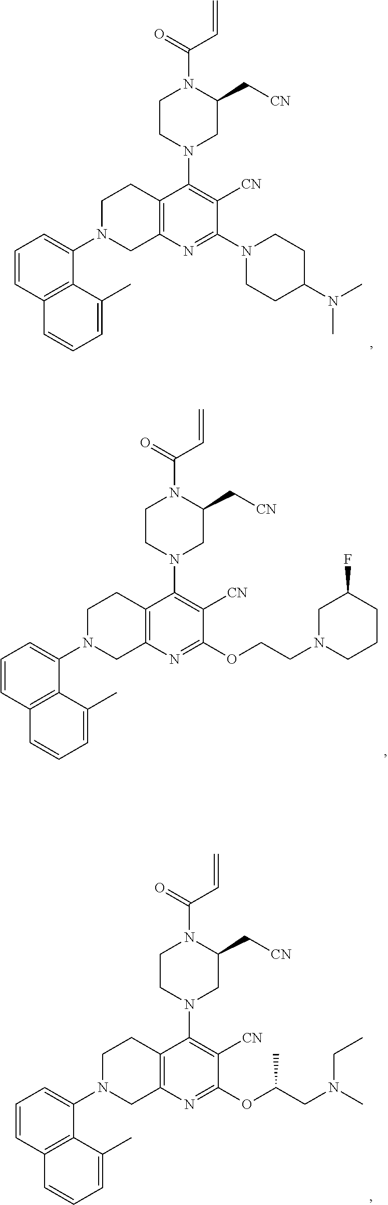 [SMILES: C=CC(=O)N1CCN(c2c(C#N)c(N3CCC(N(C)C)CC3)nc3c2CCN(c2cccc4cccc(C)c24)C3)C[C@@H]1CC#N ; C=CC(=O)N1CCN(c2c(C#N)c(OCCN3CCC[C@H](F)C3)nc3c2CCN(c2cccc4cccc(C)c24)C3)C[C@@H]1CC#N ; C=CC(=O)N1CCN(c2c(C#N)c(O[C@H](C)CN(C)CC)nc3c2CCN(c2cccc4cccc(C)c24)C3)C[C@@H]1CC#N]

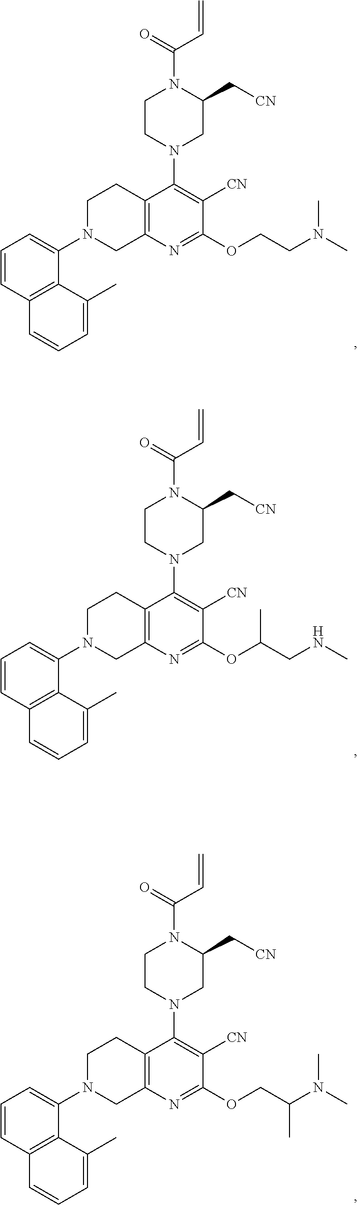 [SMILES: C=CC(=O)N1CCN(c2c(C#N)c(OCCN(C)C)nc3c2CCN(c2cccc4cccc(C)c24)C3)C[C@@H]1CC#N ; C=CC(=O)N1CCN(c2c(C#N)c(OC(C)CNC)nc3c2CCN(c2cccc4cccc(C)c24)C3)C[C@@H]1CC#N ; C=CC(=O)N1CCN(c2c(C#N)c(OCC(C)N(C)C)nc3c2CCN(c2cccc4cccc(C)c24)C3)C[C@@H]1CC#N]

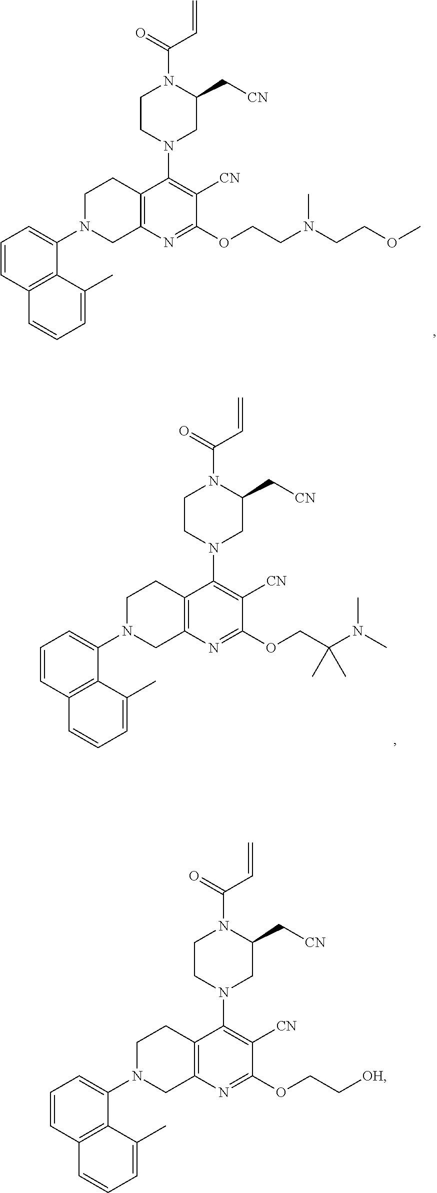 [SMILES: C=CC(=O)N1CCN(c2c(C#N)c(OCCN(C)CCOC)nc3c2CCN(c2cccc4cccc(C)c24)C3)C[C@@H]1CC#N ; C=CC(=O)N1CCN(c2c(C#N)c(OCC(C)(C)N(C)C)nc3c2CCN(c2cccc4cccc(C)c24)C3)C[C@@H]1CC#N ; C=CC(=O)N1CCN(c2c(C#N)c(OCCO)nc3c2CCN(c2cccc4cccc(C)c24)C3)C[C@@H]1CC#N]

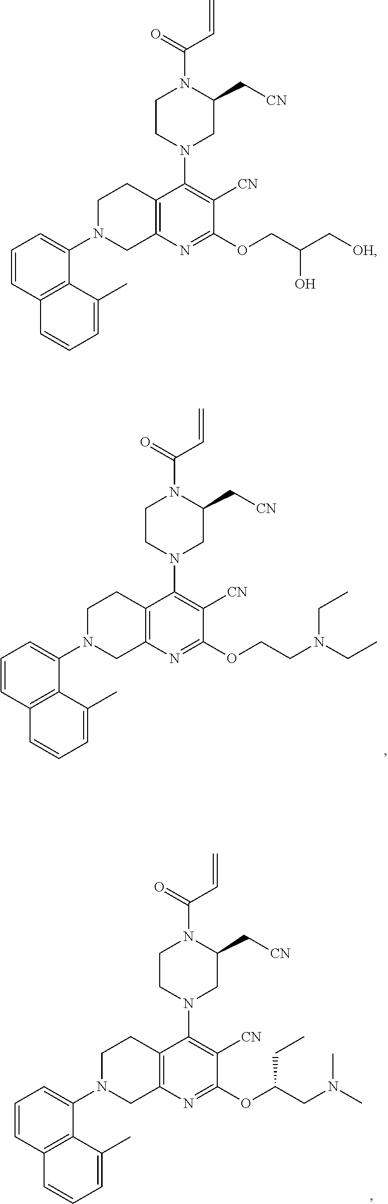 [SMILES: C=CC(=O)N1CCN(c2c(C#N)c(OCC(O)CO)nc3c2CCN(c2cccc4cccc(C)c24)C3)C[C@@H]1CC#N ; C=CC(=O)N1CCN(c2c(C#N)c(OCCN(CC)CC)nc3c2CCN(c2cccc4cccc(C)c24)C3)C[C@@H]1CC#N ; C=CC(=O)N1CCN(c2c(C#N)c(O[C@H](CC)CN(C)C)nc3c2CCN(c2cccc4cccc(C)c24)C3)C[C@@H]1CC#N]

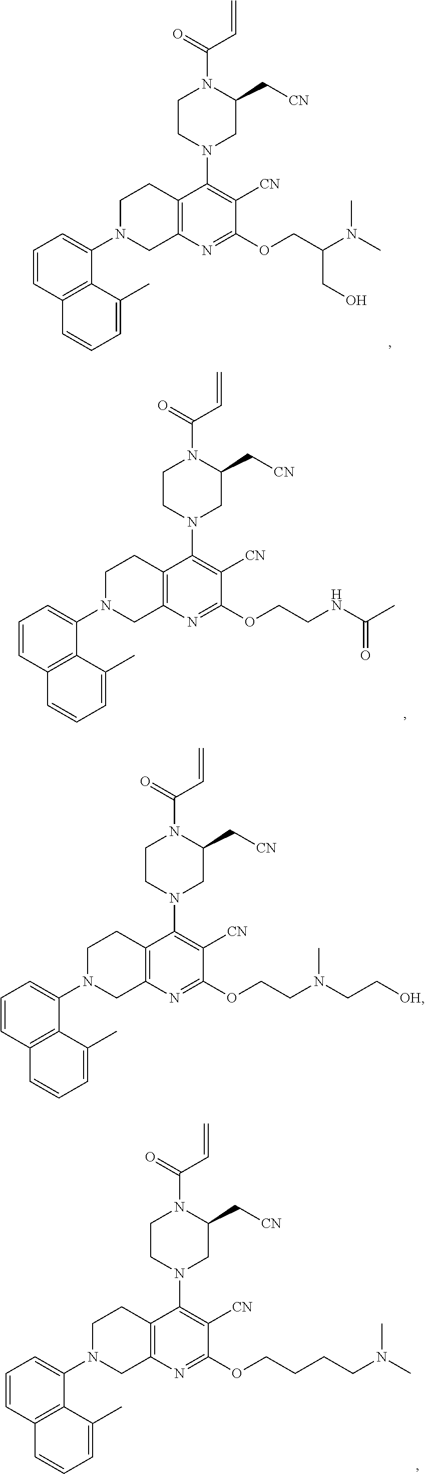 [SMILES: C=CC(=O)N1CCN(c2c(C#N)c(OCC(CO)N(C)C)nc3c2CCN(c2cccc4cccc(C)c24)C3)C[C@@H]1CC#N ; C=CC(=O)N1CCN(c2c(C#N)c(OCCNC(C)=O)nc3c2CCN(c2cccc4cccc(C)c24)C3)C[C@@H]1CC#N ; C=CC(=O)N1CCN(c2c(C#N)c(OCCN(C)CCO)nc3c2CCN(c2cccc4cccc(C)c24)C3)C[C@@H]1CC#N ; C=CC(=O)N1CCN(c2c(C#N)c(OCCCCN(C)C)nc3c2CCN(c2cccc4cccc(C)c24)C3)C[C@@H]1CC#N]

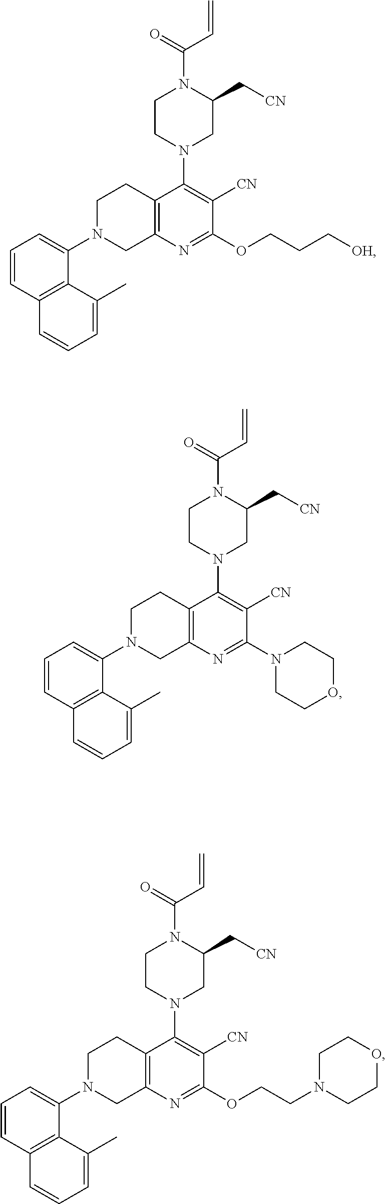 [SMILES: C=CC(=O)N1CCN(c2c(C#N)c(OCCCO)nc3c2CCN(c2cccc4cccc(C)c24)C3)C[C@@H]1CC#N ; C=CC(=O)N1CCN(c2c(C#N)c(N3CCOCC3)nc3c2CCN(c2cccc4cccc(C)c24)C3)C[C@@H]1CC#N ; C=CC(=O)N1CCN(c2c(C#N)c(OCCN3CCOCC3)nc3c2CCN(c2cccc4cccc(C)c24)C3)C[C@@H]1CC#N]

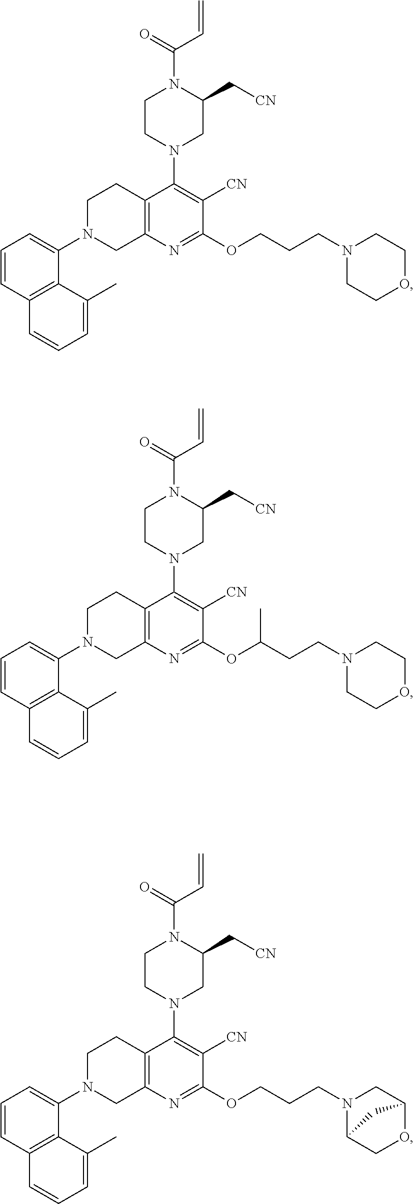 [SMILES: C=CC(=O)N1CCN(c2c(C#N)c(OCCCN3CCOCC3)nc3c2CCN(c2cccc4cccc(C)c24)C3)C[C@@H]1CC#N ; C=CC(=O)N1CCN(c2c(C#N)c(OC(C)CCN3CCOCC3)nc3c2CCN(c2cccc4cccc(C)c24)C3)C[C@@H]1CC#N ; C=CC(=O)N1CCN(c2c(C#N)c(OCCCN3C[C@@H]4C[C@H]3CO4)nc3c2CCN(c2cccc4cccc(C)c24)C3)C[C@@H]1CC#N]

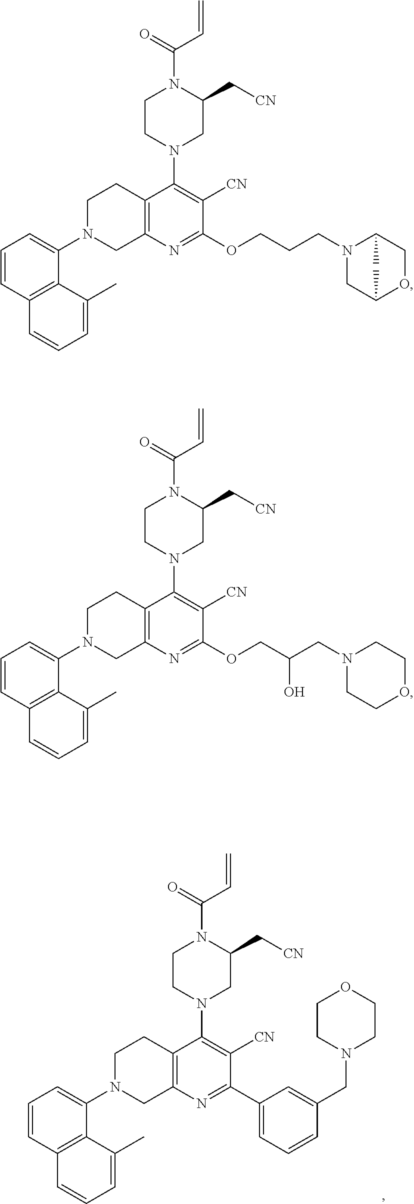 [SMILES: C=CC(=O)N1CCN(c2c(C#N)c(OCCCN3C[C@H]4C[C@@H]3CO4)nc3c2CCN(c2cccc4cccc(C)c24)C3)C[C@@H]1CC#N ; C=CC(=O)N1CCN(c2c(C#N)c(OCC(O)CN3CCOCC3)nc3c2CCN(c2cccc4cccc(C)c24)C3)C[C@@H]1CC#N ; C=CC(=O)N1CCN(c2c(C#N)c(-c3cccc(CN4CCOCC4)c3)nc3c2CCN(c2cccc4cccc(C)c24)C3)C[C@@H]1CC#N]

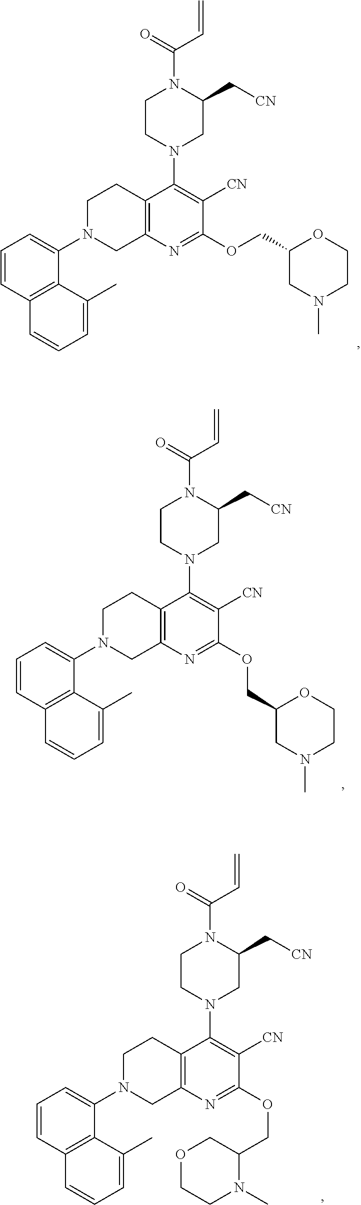 [SMILES: C=CC(=O)N1CCN(c2c(C#N)c(OC[C@H]3CN(C)CCO3)nc3c2CCN(c2cccc4cccc(C)c24)C3)C[C@@H]1CC#N ; C=CC(=O)N1CCN(c2c(C#N)c(OC[C@@H]3CN(C)CCO3)nc3c2CCN(c2cccc4cccc(C)c24)C3)C[C@@H]1CC#N ; C=CC(=O)N1CCN(c2c(C#N)c(OCC3COCCN3C)nc3c2CCN(c2cccc4cccc(C)c24)C3)C[C@@H]1CC#N]

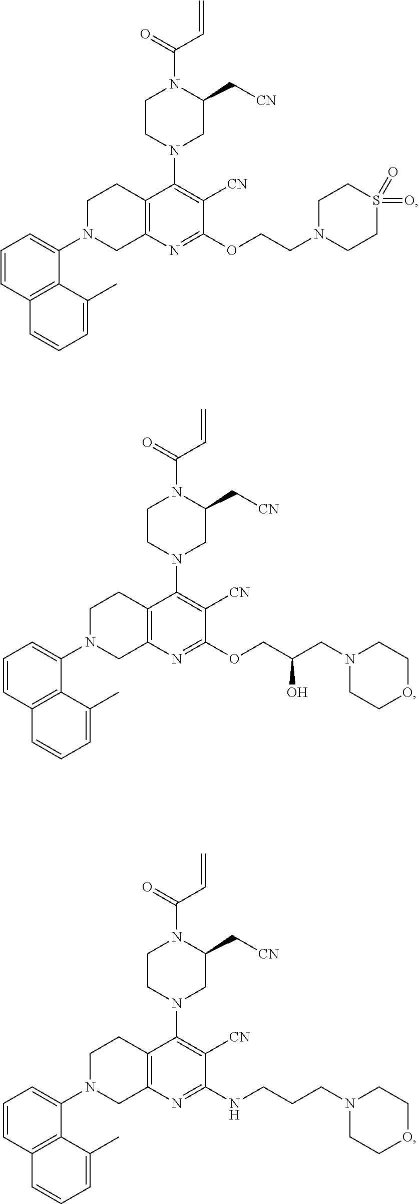 [SMILES: C=CC(=O)N1CCN(c2c(C#N)c(OCCN3CCS(=O)(=O)CC3)nc3c2CCN(c2cccc4cccc(C)c24)C3)C[C@@H]1CC#N ; C=CC(=O)N1CCN(c2c(C#N)c(OC[C@H](O)CN3CCOCC3)nc3c2CCN(c2cccc4cccc(C)c24)C3)C[C@@H]1CC#N ; C=CC(=O)N1CCN(c2c(C#N)c(NCCCN3CCOCC3)nc3c2CCN(c2cccc4cccc(C)c24)C3)C[C@@H]1CC#N]

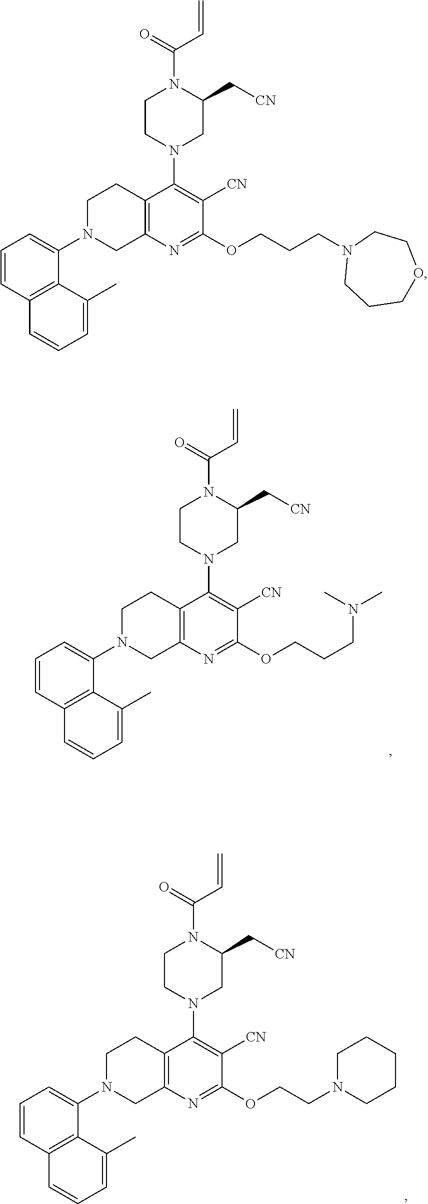 [SMILES: C=CC(=O)N1CCN(c2c(C#N)c(OCCCN3CCCOCC3)nc3c2CCN(c2cccc4cccc(C)c24)C3)C[C@@H]1CC#N ; C=CC(=O)N1CCN(c2c(C#N)c(OCCCN(C)C)nc3c2CCN(c2cccc4cccc(C)c24)C3)C[C@@H]1CC#N ; C=CC(=O)N1CCN(c2c(C#N)c(OCCN3CCCCC3)nc3c2CCN(c2cccc4cccc(C)c24)C3)C[C@@H]1CC#N]

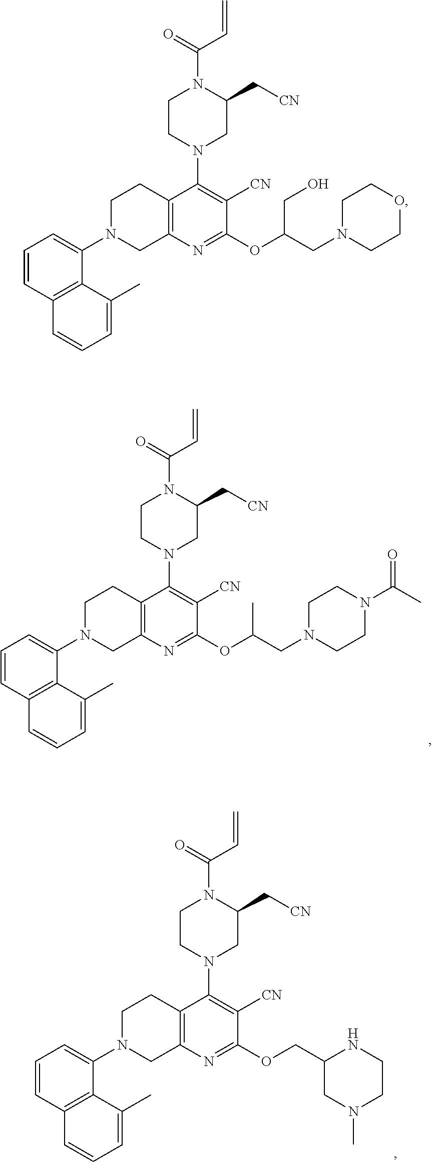 [SMILES: C=CC(=O)N1CCN(c2c(C#N)c(OC(CO)CN3CCOCC3)nc3c2CCN(c2cccc4cccc(C)c24)C3)C[C@@H]1CC#N ; C=CC(=O)N1CCN(c2c(C#N)c(OC(C)CN3CCN(C(C)=O)CC3)nc3c2CCN(c2cccc4cccc(C)c24)C3)C[C@@H]1CC#N ; C=CC(=O)N1CCN(c2c(C#N)c(OCC3CN(C)CCN3)nc3c2CCN(c2cccc4cccc(C)c24)C3)C[C@@H]1CC#N]

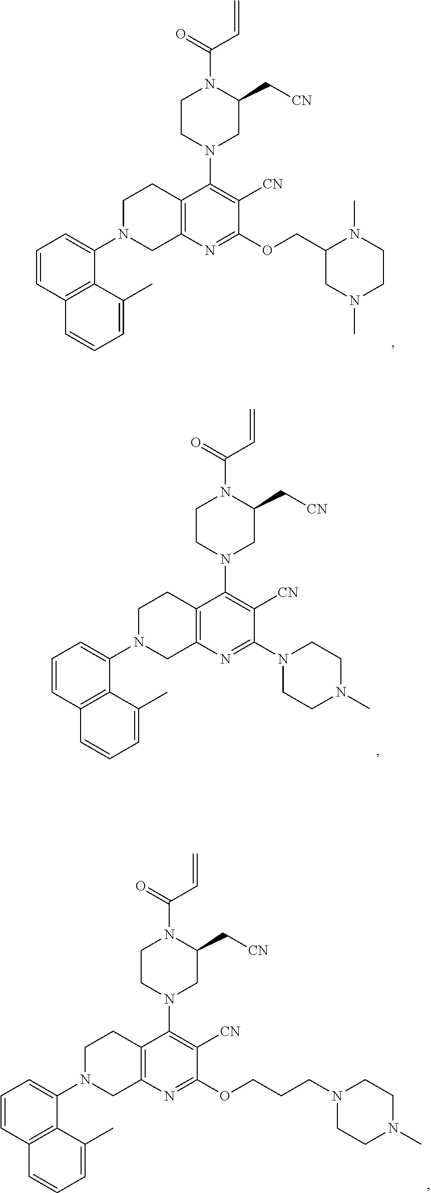 [SMILES: C=CC(=O)N1CCN(c2c(C#N)c(OCC3CN(C)CCN3C)nc3c2CCN(c2cccc4cccc(C)c24)C3)C[C@@H]1CC#N ; C=CC(=O)N1CCN(c2c(C#N)c(N3CCN(C)CC3)nc3c2CCN(c2cccc4cccc(C)c24)C3)C[C@@H]1CC#N ; C=CC(=O)N1CCN(c2c(C#N)c(OCCCN3CCN(C)CC3)nc3c2CCN(c2cccc4cccc(C)c24)C3)C[C@@H]1CC#N]

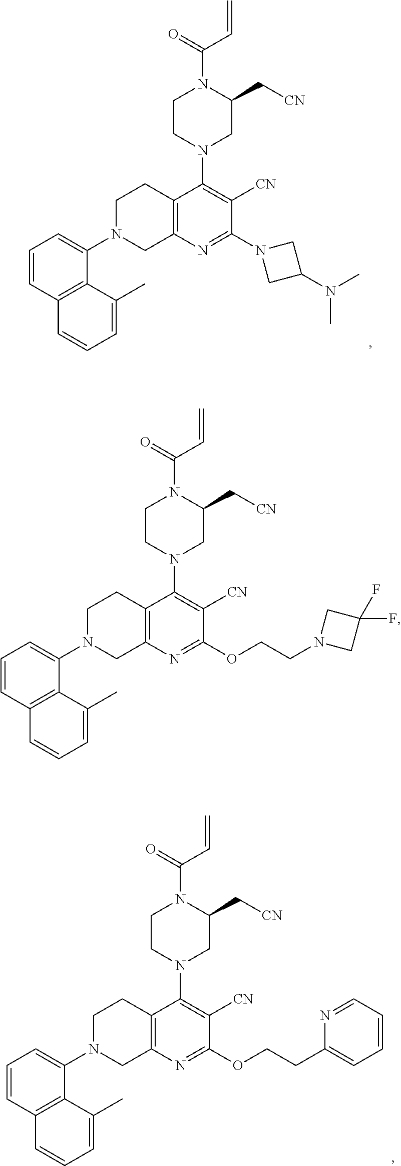 [SMILES: C=CC(=O)N1CCN(c2c(C#N)c(N3CC(N(C)C)C3)nc3c2CCN(c2cccc4cccc(C)c24)C3)C[C@@H]1CC#N ; C=CC(=O)N1CCN(c2c(C#N)c(OCCN3CC(F)(F)C3)nc3c2CCN(c2cccc4cccc(C)c24)C3)C[C@@H]1CC#N ; C=CC(=O)N1CCN(c2c(C#N)c(OCCc3ccccn3)nc3c2CCN(c2cccc4cccc(C)c24)C3)C[C@@H]1CC#N]

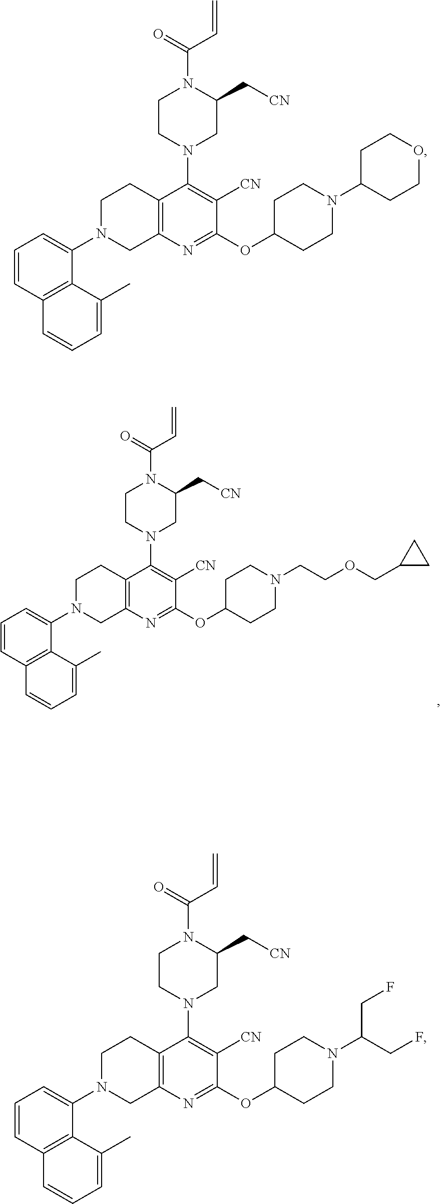 [SMILES: C=CC(=O)N1CCN(c2c(C#N)c(OC3CCN(C4CCOCC4)CC3)nc3c2CCN(c2cccc4cccc(C)c24)C3)C[C@@H]1CC#N ; C=CC(=O)N1CCN(c2c(C#N)c(OC3CCN(CCOCC4CC4)CC3)nc3c2CCN(c2cccc4cccc(C)c24)C3)C[C@@H]1CC#N ; C=CC(=O)N1CCN(c2c(C#N)c(OC3CCN(C(CF)CF)CC3)nc3c2CCN(c2cccc4cccc(C)c24)C3)C[C@@H]1CC#N]

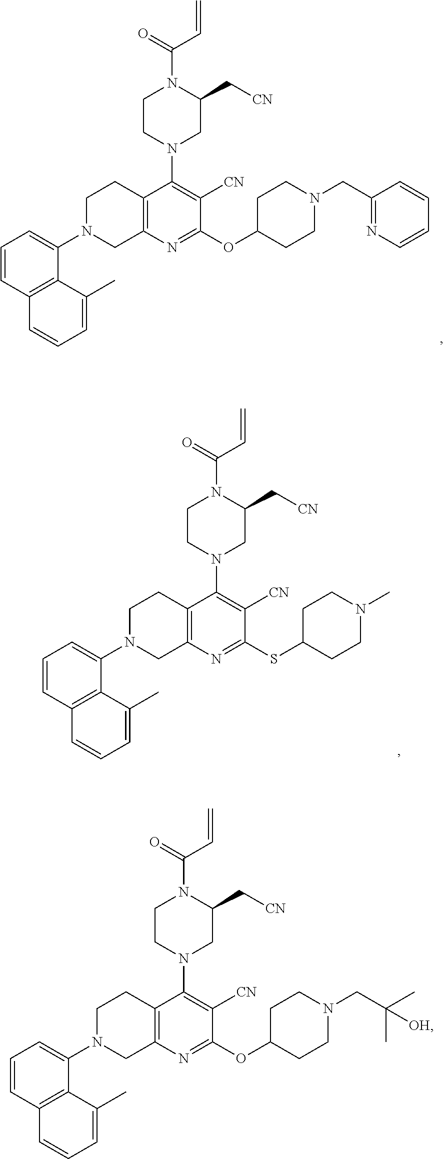 [SMILES: C=CC(=O)N1CCN(c2c(C#N)c(OC3CCN(Cc4ccccn4)CC3)nc3c2CCN(c2cccc4cccc(C)c24)C3)C[C@@H]1CC#N ; C=CC(=O)N1CCN(c2c(C#N)c(SC3CCN(C)CC3)nc3c2CCN(c2cccc4cccc(C)c24)C3)C[C@@H]1CC#N ; C=CC(=O)N1CCN(c2c(C#N)c(OC3CCN(CC(C)(C)O)CC3)nc3c2CCN(c2cccc4cccc(C)c24)C3)C[C@@H]1CC#N]

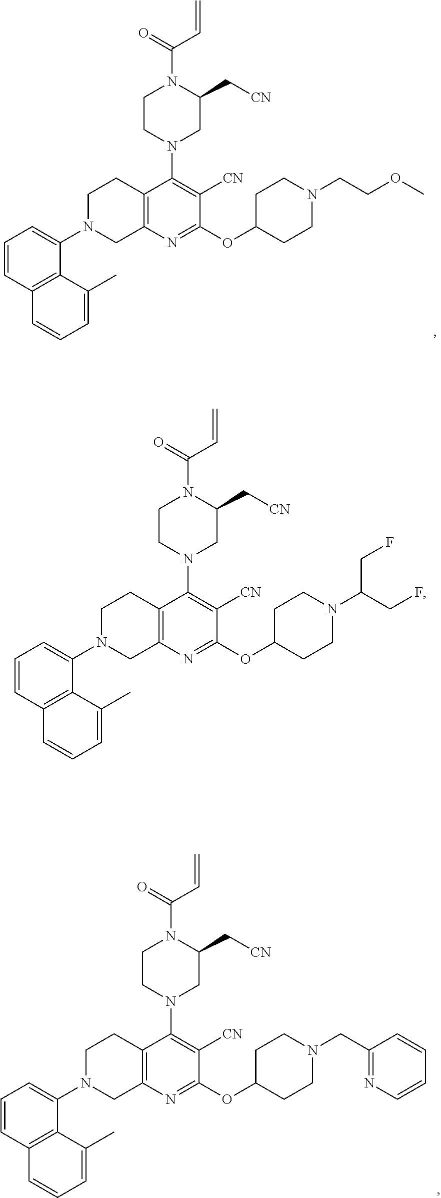 [SMILES: C=CC(=O)N1CCN(c2c(C#N)c(OC3CCN(CCOC)CC3)nc3c2CCN(c2cccc4cccc(C)c24)C3)C[C@@H]1CC#N ; C=CC(=O)N1CCN(c2c(C#N)c(OC3CCN(C(CF)CF)CC3)nc3c2CCN(c2cccc4cccc(C)c24)C3)C[C@@H]1CC#N ; C=CC(=O)N1CCN(c2c(C#N)c(OC3CCN(Cc4ccccn4)CC3)nc3c2CCN(c2cccc4cccc(C)c24)C3)C[C@@H]1CC#N]

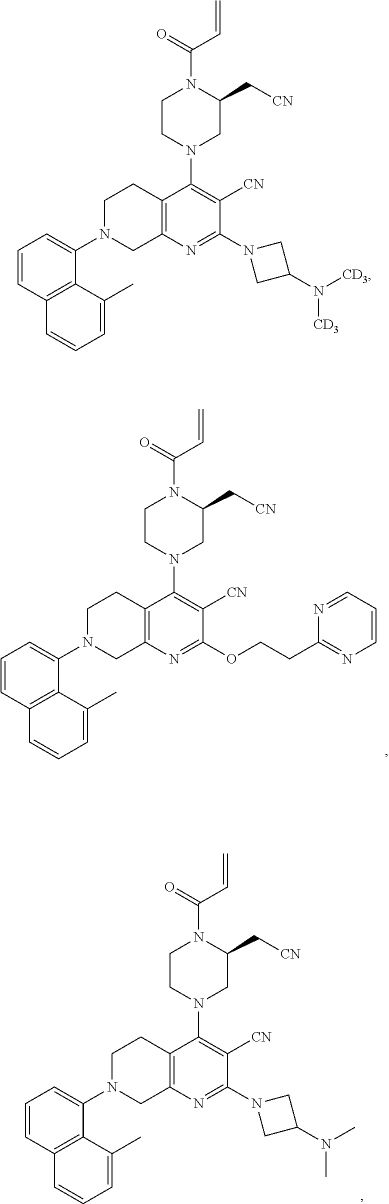 [SMILES: [2H]C([2H])([2H])N(C1CN(c2nc3c(c(N4CCN(C(=O)C=C)[C@@H](CC#N)C4)c2C#N)CCN(c2cccc4cccc(C)c24)C3)C1)C([2H])([2H])[2H] ; C=CC(=O)N1CCN(c2c(C#N)c(OCCc3ncccn3)nc3c2CCN(c2cccc4cccc(C)c24)C3)C[C@@H]1CC#N ; C=CC(=O)N1CCN(c2c(C#N)c(N3CC(N(C)C)C3)nc3c2CCN(c2cccc4cccc(C)c24)C3)C[C@@H]1CC#N]

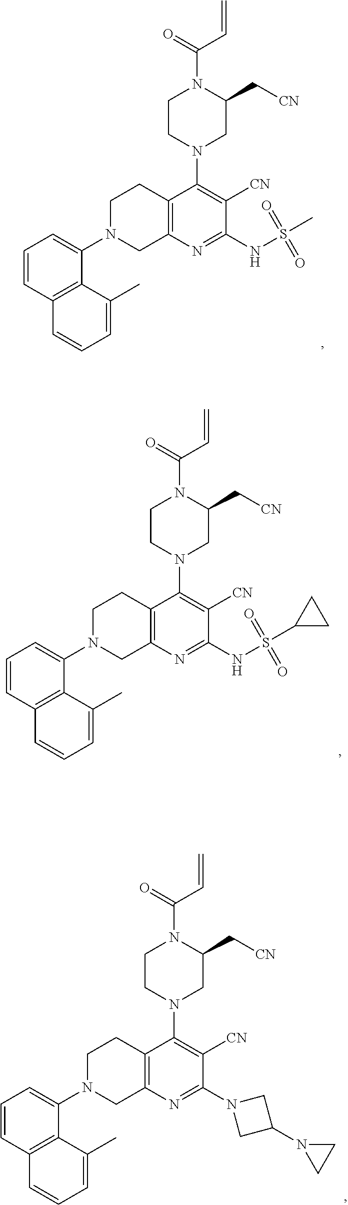 [SMILES: C=CC(=O)N1CCN(c2c(C#N)c(NS(C)(=O)=O)nc3c2CCN(c2cccc4cccc(C)c24)C3)C[C@@H]1CC#N ; C=CC(=O)N1CCN(c2c(C#N)c(NS(=O)(=O)C3CC3)nc3c2CCN(c2cccc4cccc(C)c24)C3)C[C@@H]1CC#N ; C=CC(=O)N1CCN(c2c(C#N)c(N3CC(N4CC4)C3)nc3c2CCN(c2cccc4cccc(C)c24)C3)C[C@@H]1CC#N]

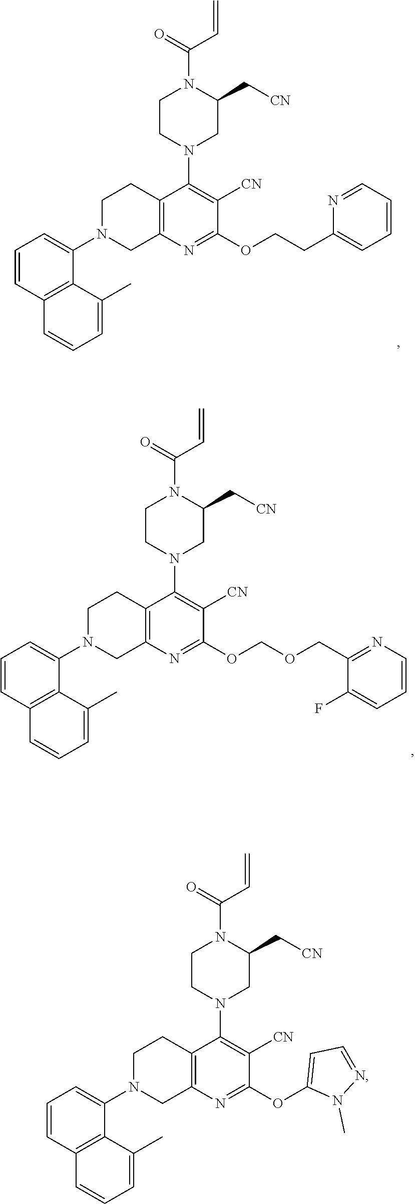 [SMILES: C=CC(=O)N1CCN(c2c(C#N)c(OCCc3ccccn3)nc3c2CCN(c2cccc4cccc(C)c24)C3)C[C@@H]1CC#N ; C=CC(=O)N1CCN(c2c(C#N)c(OCOCc3ncccc3F)nc3c2CCN(c2cccc4cccc(C)c24)C3)C[C@@H]1CC#N ; C=CC(=O)N1CCN(c2c(C#N)c(Oc3ccnn3C)nc3c2CCN(c2cccc4cccc(C)c24)C3)C[C@@H]1CC#N]

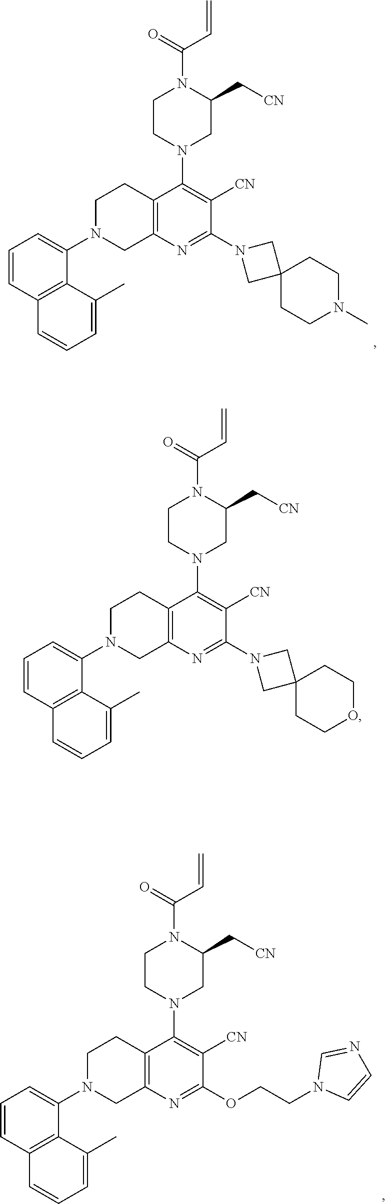 [SMILES: C=CC(=O)N1CCN(c2c(C#N)c(N3CC4(CCN(C)CC4)C3)nc3c2CCN(c2cccc4cccc(C)c24)C3)C[C@@H]1CC#N ; C=CC(=O)N1CCN(c2c(C#N)c(N3CC4(CCOCC4)C3)nc3c2CCN(c2cccc4cccc(C)c24)C3)C[C@@H]1CC#N ; C=CC(=O)N1CCN(c2c(C#N)c(OCCn3ccnc3)nc3c2CCN(c2cccc4cccc(C)c24)C3)C[C@@H]1CC#N]

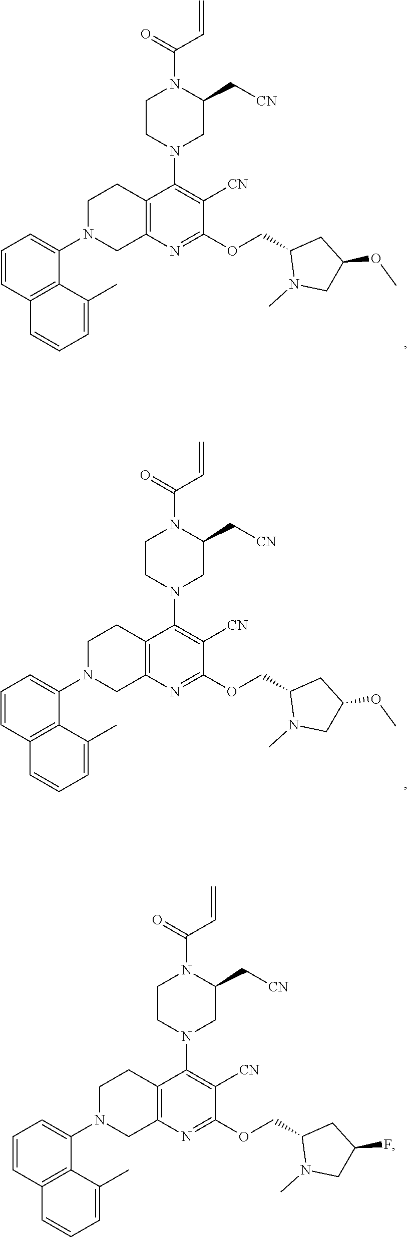 [SMILES: C=CC(=O)N1CCN(c2c(C#N)c(OC[C@@H]3C[C@@H](OC)CN3C)nc3c2CCN(c2cccc4cccc(C)c24)C3)C[C@@H]1CC#N ; C=CC(=O)N1CCN(c2c(C#N)c(OC[C@@H]3C[C@H](OC)CN3C)nc3c2CCN(c2cccc4cccc(C)c24)C3)C[C@@H]1CC#N ; C=CC(=O)N1CCN(c2c(C#N)c(OC[C@@H]3C[C@@H](F)CN3C)nc3c2CCN(c2cccc4cccc(C)c24)C3)C[C@@H]1CC#N]

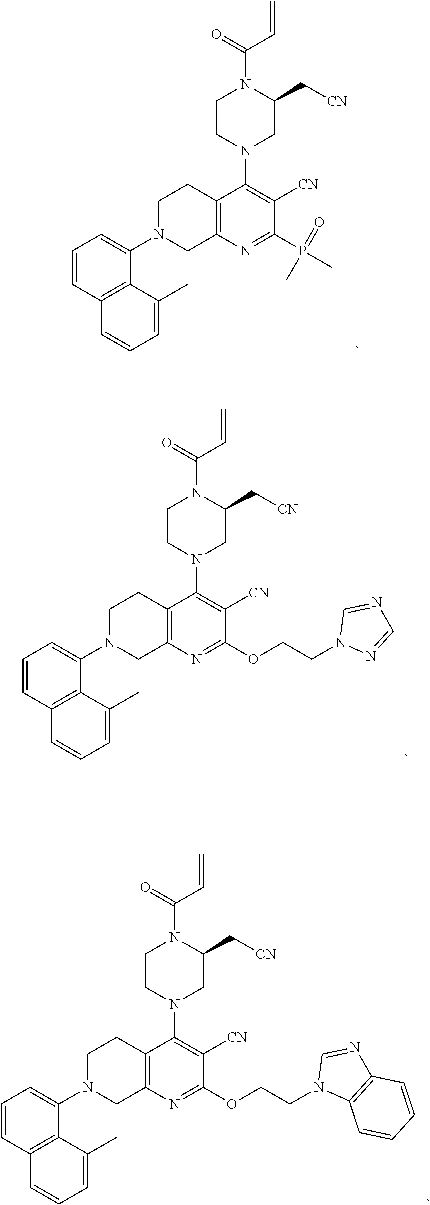 [SMILES: C=CC(=O)N1CCN(c2c(C#N)c(P(C)(C)=O)nc3c2CCN(c2cccc4cccc(C)c24)C3)C[C@@H]1CC#N ; C=CC(=O)N1CCN(c2c(C#N)c(OCCn3cncn3)nc3c2CCN(c2cccc4cccc(C)c24)C3)C[C@@H]1CC#N ; C=CC(=O)N1CCN(c2c(C#N)c(OCCn3cnc4ccccc43)nc3c2CCN(c2cccc4cccc(C)c24)C3)C[C@@H]1CC#N]

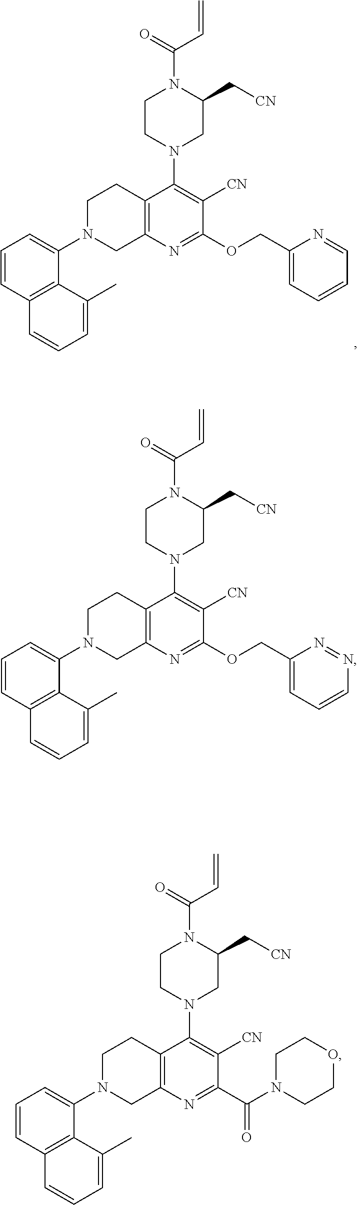 [SMILES: C=CC(=O)N1CCN(c2c(C#N)c(OCc3ccccn3)nc3c2CCN(c2cccc4cccc(C)c24)C3)C[C@@H]1CC#N ; C=CC(=O)N1CCN(c2c(C#N)c(OCc3cccnn3)nc3c2CCN(c2cccc4cccc(C)c24)C3)C[C@@H]1CC#N ; C=CC(=O)N1CCN(c2c(C#N)c(C(=O)N3CCOCC3)nc3c2CCN(c2cccc4cccc(C)c24)C3)C[C@@H]1CC#N]

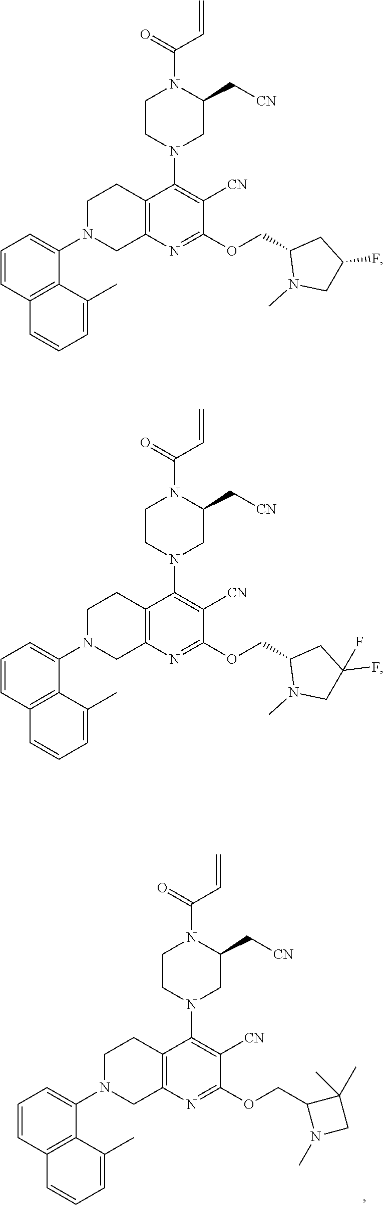 [SMILES: C=CC(=O)N1CCN(c2c(C#N)c(OC[C@@H]3C[C@H](F)CN3C)nc3c2CCN(c2cccc4cccc(C)c24)C3)C[C@@H]1CC#N ; C=CC(=O)N1CCN(c2c(C#N)c(OC[C@@H]3CC(F)(F)CN3C)nc3c2CCN(c2cccc4cccc(C)c24)C3)C[C@@H]1CC#N ; C=CC(=O)N1CCN(c2c(C#N)c(OCC3N(C)CC3(C)C)nc3c2CCN(c2cccc4cccc(C)c24)C3)C[C@@H]1CC#N]

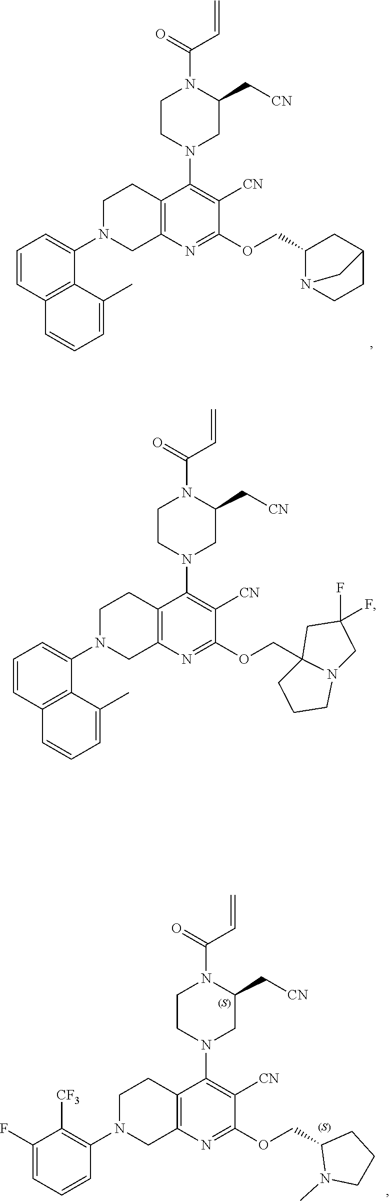

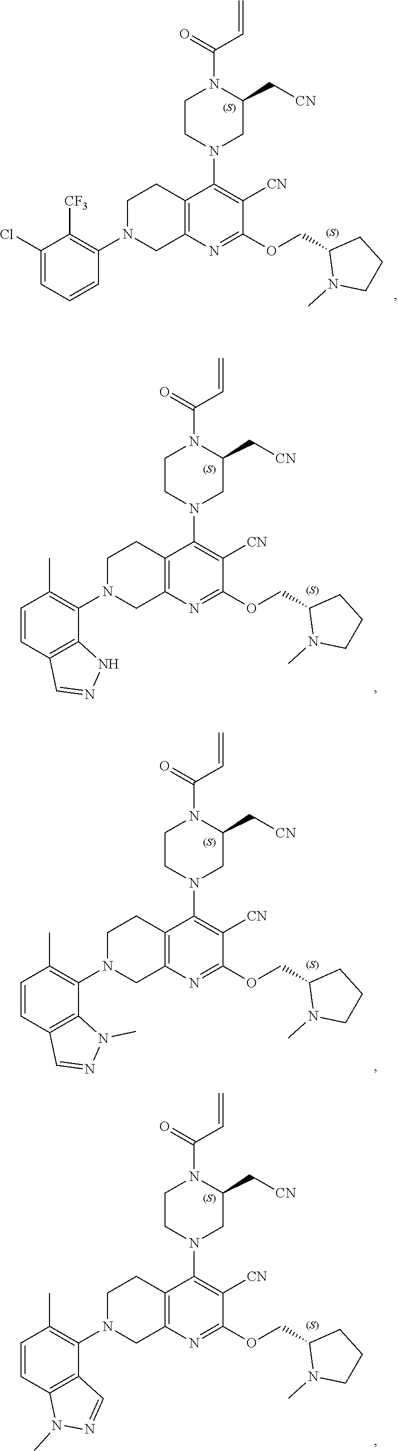

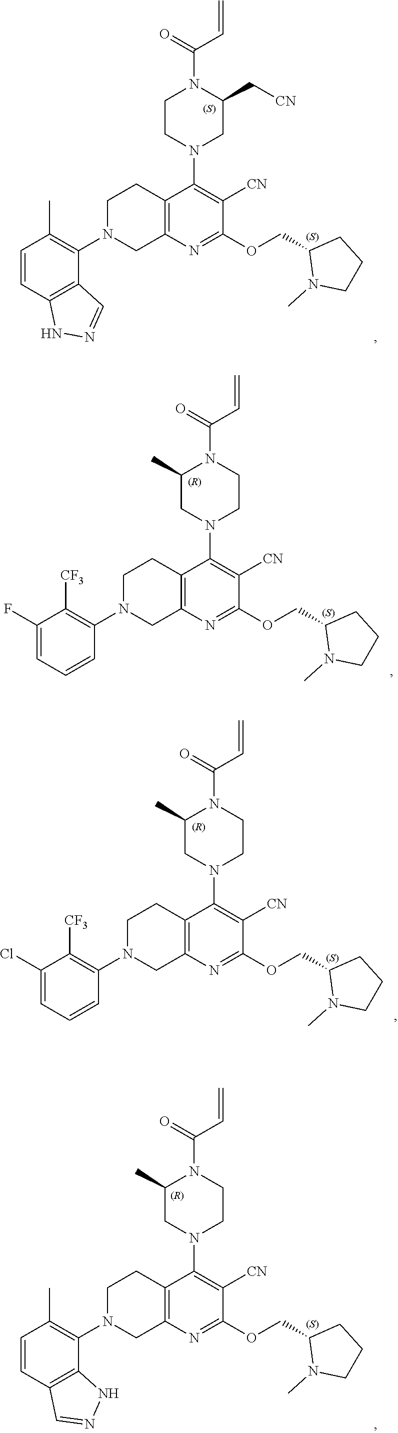

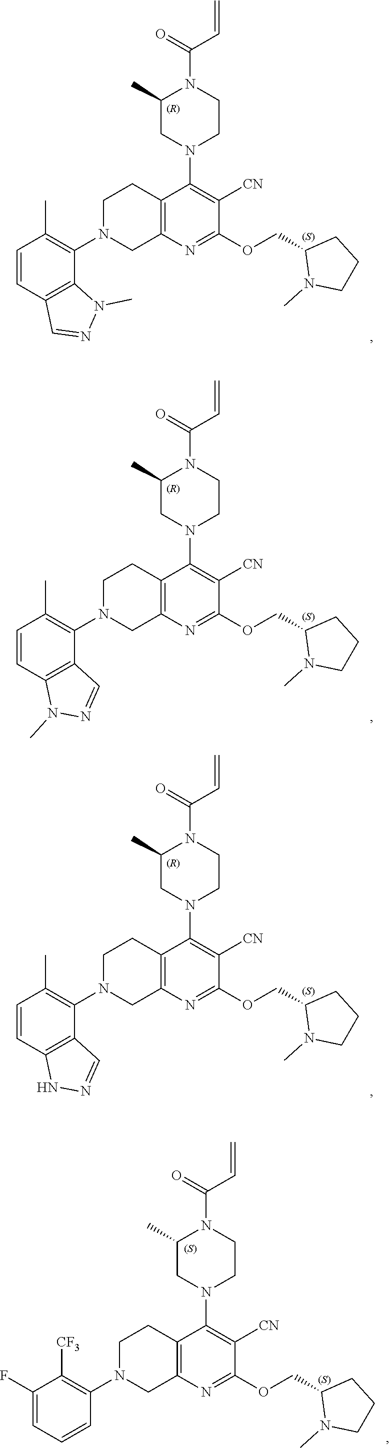

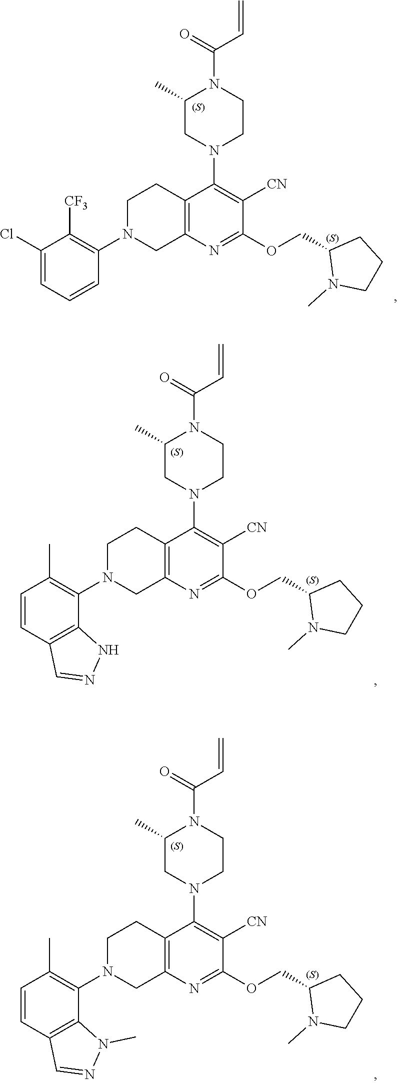

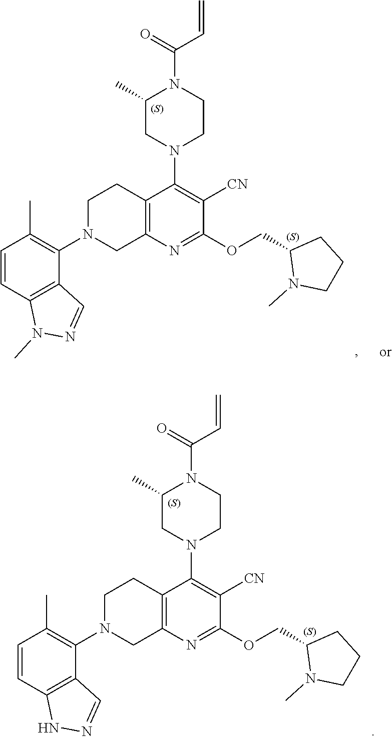

In some embodiments, the compound is:

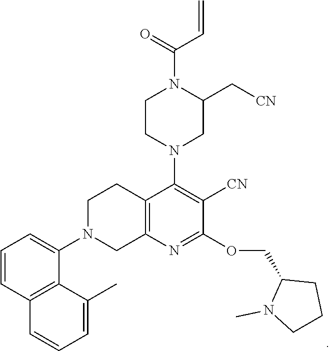 [SMILES: C=CC(=O)N1CCN(c2c(C#N)c(OC[C@@H]3CCCN3C)nc3c2CCN(c2cccc4cccc(C)c24)C3)CC1CC#N]

In some embodiments, the compound is:

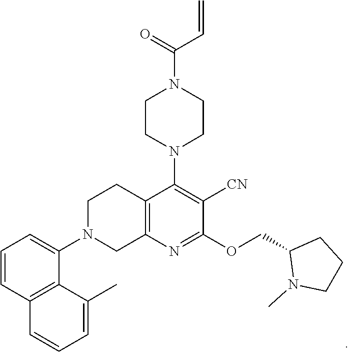 [SMILES: C=CC(=O)N1CCN(c2c(C#N)c(OC[C@@H]3CCCN3C)nc3c2CCN(c2cccc4cccc(C)c24)C3)CC1]

In some embodiments, the compound is:

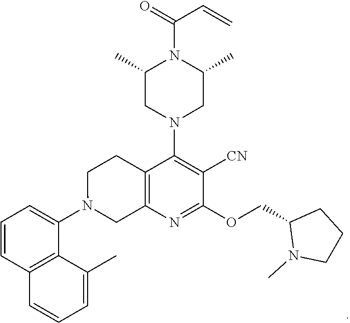 [SMILES: C=CC(=O)N1[C@H](C)CN(c2c(C#N)c(OC[C@@H]3CCCN3C)nc3c2CCN(c2cccc4cccc(C)c24)C3)C[C@@H]1C]

In some embodiments, the compound is:

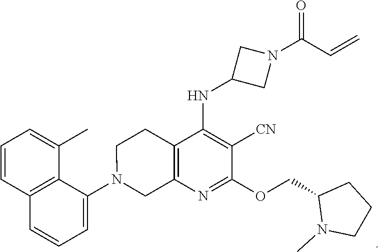 [SMILES: C=CC(=O)N1CC(Nc2c(C#N)c(OC[C@@H]3CCCN3C)nc3c2CCN(c2cccc4cccc(C)c24)C3)C1]

In some embodiments, the compound is:

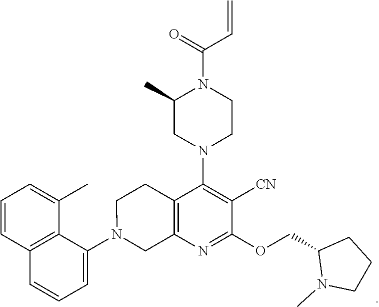 [SMILES: C=CC(=O)N1CCN(c2c(C#N)c(OC[C@@H]3CCCN3C)nc3c2CCN(c2cccc4cccc(C)c24)C3)C[C@H]1C]

In another aspect, there is provided a pharmaceutical composition comprising at least one compound of formula (I), a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of the present invention, and at least one pharmaceutically acceptable excipient. In some embodiments, the said compound in a weight ratio to the said excipient within the range from about 0.0001 to about 10. In some embodiments, the said compound in a weight ratio to the said excipient within the range from about 0.01 to about 0.8. In some embodiments, the said compound in a weight ratio to the said excipient within the range from about 0.02 to about 0.2. In some embodiments, the said compound in a weight ratio to the said excipient within the range from about 0.05 to about 0.15.

In some embodiments, the compound is:

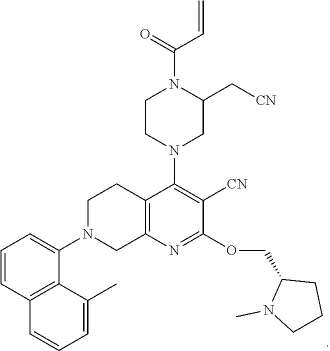 [SMILES: C=CC(=O)N1CCN(c2c(C#N)c(OC[C@@H]3CCCN3C)nc3c2CCN(c2cccc4cccc(C)c24)C3)CC1CC#N]

In some embodiments, the compound is:

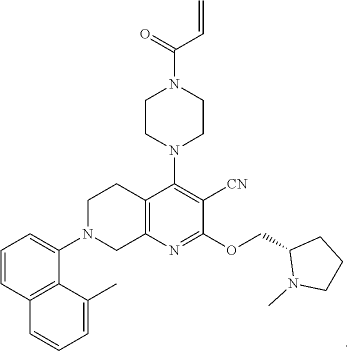 [SMILES: C=CC(=O)N1CCN(c2c(C#N)c(OC[C@@H]3CCCN3C)nc3c2CCN(c2cccc4cccc(C)c24)C3)CC1]

In some embodiments, the compound is:

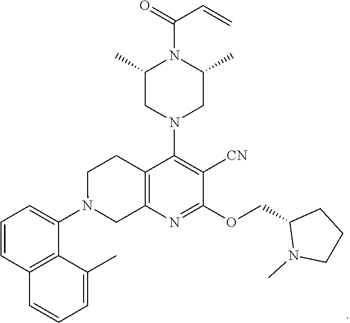 [SMILES: C=CC(=O)N1[C@H](C)CN(c2c(C#N)c(OC[C@@H]3CCCN3C)nc3c2CCN(c2cccc4cccc(C)c24)C3)C[C@@H]1C]

In some embodiments, the compound is:

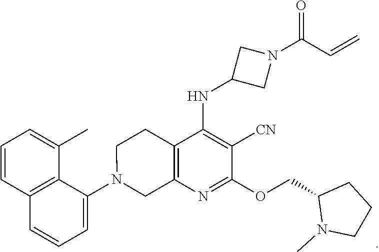 [SMILES: C=CC(=O)N1CC(Nc2c(C#N)c(OC[C@@H]3CCCN3C)nc3c2CCN(c2cccc4cccc(C)c24)C3)C1]

In some embodiments, the compound is:

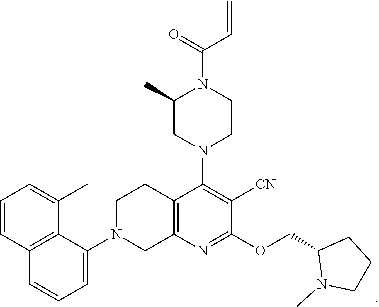 [SMILES: C=CC(=O)N1CCN(c2c(C#N)c(OC[C@@H]3CCCN3C)nc3c2CCN(c2cccc4cccc(C)c24)C3)C[C@H]1C]

In another aspect, there is provided that use of the compound of formula (I), a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of the present invention or the pharmaceutical composition of the present invention for the manufacture of a medicament for the treatment of diseases or conditions related to KRAS mutant protein. In some embodiments, the diseases or conditions related to KRAS mutant protein is the diseases or conditions related to KRAS G12C mutant protein. In some embodiments, the diseases or conditions related to KRAS mutant protein is cancer related to KRAS mutant protein. In some embodiments, the cancer is selected from blood cancer, pancreatic cancer, colon cancer, rectal cancer, colorectal cancer or lung cancer. In some embodiments, the blood cancer is selected from acute myeloid leukemia or acute lymphocytic leukemia; the lung cancer is selected from non-small cell lung cancer or small cell lung cancer.

In some embodiments, the compound is:

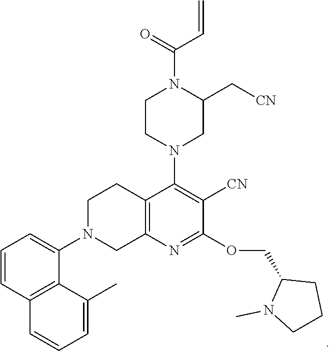 [SMILES: C=CC(=O)N1CCN(c2c(C#N)c(OC[C@@H]3CCCN3C)nc3c2CCN(c2cccc4cccc(C)c24)C3)CC1CC#N]

In some embodiments, the compound is:

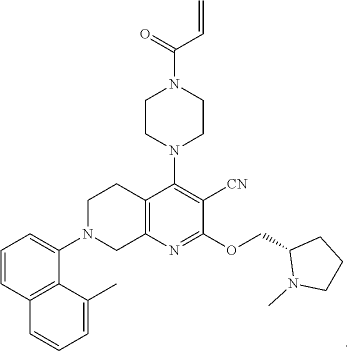 [SMILES: C=CC(=O)N1CCN(c2c(C#N)c(OC[C@@H]3CCCN3C)nc3c2CCN(c2cccc4cccc(C)c24)C3)CC1]

In some embodiments, the compound is:

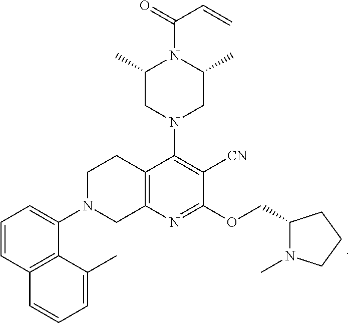 [SMILES: C=CC(=O)N1[C@H](C)CN(c2c(C#N)c(OC[C@@H]3CCCN3C)nc3c2CCN(c2cccc4cccc(C)c24)C3)C[C@@H]1C]

In some embodiments, the compound is:

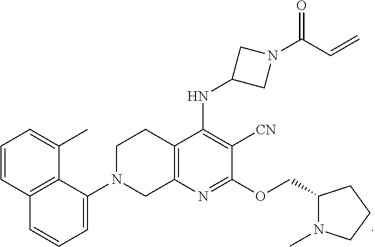 [SMILES: C=CC(=O)N1CC(Nc2c(C#N)c(OC[C@@H]3CCCN3C)nc3c2CCN(c2cccc4cccc(C)c24)C3)C1]

In some embodiments, the compound is:

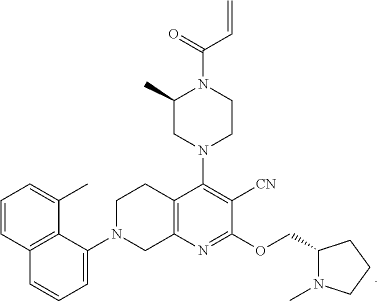 [SMILES: C=CC(=O)N1CCN(c2c(C#N)c(OC[C@@H]3CCCN3C)nc3c2CCN(c2cccc4cccc(C)c24)C3)C[C@H]1C]

In another aspect, there is provided that a method of treating a patient having a diseases or conditions related to KRAS mutant protein, said method comprising administering to the subject a therapeutically effective amount of at least one compound of formula (I), a stereoisomer thereof, an atropisomer thereof, a pharmaceutically acceptable salt thereof, a pharmaceutically acceptable salt of the stereoisomer thereof or a pharmaceutically acceptable salt of the atropisomer thereof of the present invention; or the pharmaceutical composition of the present invention. In some embodiments, the diseases or conditions related to KRAS mutant protein is the diseases or conditions related to KRAS G12C mutant protein. In some embodiments, the diseases or conditions related to KRAS mutant protein is cancer related to KRAS mutant protein. In some embodiments, the cancer is selected from blood cancer, pancreatic cancer, colon cancer, rectal cancer, colorectal cancer or lung cancer. In some embodiments, the blood cancer is selected from acute myeloid leukemia or acute lymphocytic leukemia; the lung cancer is selected from non-small cell lung cancer or small cell lung cancer.

In some embodiments, the compound is:

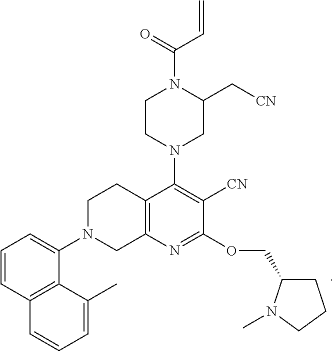 [SMILES: C=CC(=O)N1CCN(c2c(C#N)c(OC[C@@H]3CCCN3C)nc3c2CCN(c2cccc4cccc(C)c24)C3)CC1CC#N]

In some embodiments, the compound is:

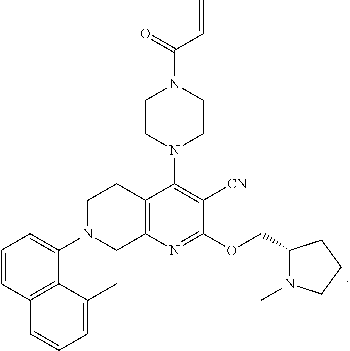 [SMILES: C=CC(=O)N1CCN(c2c(C#N)c(OC[C@@H]3CCCN3C)nc3c2CCN(c2cccc4cccc(C)c24)C3)CC1]

In some embodiments, the compound is:

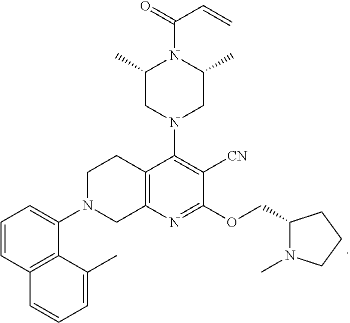 [SMILES: C=CC(=O)N1[C@H](C)CN(c2c(C#N)c(OC[C@@H]3CCCN3C)nc3c2CCN(c2cccc4cccc(C)c24)C3)C[C@@H]1C]

In some embodiments, the compound is:

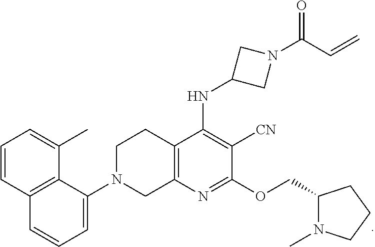 [SMILES: C=CC(=O)N1CC(Nc2c(C#N)c(OC[C@@H]3CCCN3C)nc3c2CCN(c2cccc4cccc(C)c24)C3)C1]

In some embodiments, the compound is:

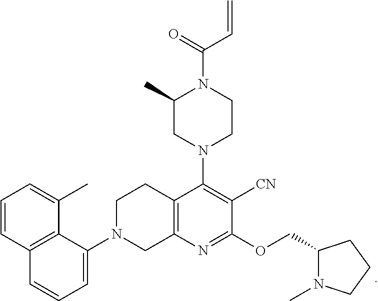 [SMILES: C=CC(=O)N1CCN(c2c(C#N)c(OC[C@@H]3CCCN3C)nc3c2CCN(c2cccc4cccc(C)c24)C3)C[C@H]1C]

### Definition

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similary, C₁₋₆, as in C₁₋₆alkyl is defined to identify the group as having 1, 2, 3, 4, 5 or 6 carbon atoms in a linear or branched arrangement.

The term “alkylene” means a difunctional group obtained by removal of a hydrogen atom from an alkyl group that is defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, hepetenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The term “heterocyclic”, as used herein, unless otherwise indicated, refers to unsubstituted and substituted mono or polycyclic non-aromatic ring system containing one or more heteroatoms, which comprising moncyclic heterocyclic ring, bicyclic heterocyclic ring, bridged heterocyclic ring, fused heterocyclic ring or sipro heterocyclic ring. Preferred heteroatoms include N, O, and S, including N-oxides, sulfur oxides, and dioxides. Preferably the ring is three to ten membered and is either fully saturated or has one or more degrees of unsaturation. Multiple degrees of substitution, preferably one, two or three, are included within the present definition. Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “carbocyclic” refers to a substituted or unsubstituted monocyclic ring, bicyclic ring bridged ring, fused ring, sipiro ring non-aromatic ring system only containing carbon atoms. Exemplary “cycloalkyl” groups includes but not limited to, cyclopropyl, cyclobutyl, cyclopentyl, cyclohexyl and so on.

The term “oxo” refers to oxygen atom together with the attached carbon atom forms the group

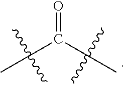 [SMILES: *C(*)=O]

The term “—C₁₋₆alkyleneC₆₋₁₀aryl” refers to the —C₁₋₆alkyl as defined above substituted by C₆₋₁₀aryl as defined above.

The term “—C₁₋₆alkylene-(5-10 membered heteroaryl)” refers to the —C₁₋₆alkyl as defined above substituted by 5-10 membered heteroaryl as defined above.

The term “—C₁₋₆alkylene-(3-10 membered heterocyclic)” refers to the —C₁₋₆alkyl as defined above substituted by 3-10 membered heterocyclic as defined above.

The term “—C₁₋₆alkylene-C₃₋₁₀carbocyclic” refers to the —C₁₋₆alkyl as defined above substituted by C₃₋₁₀carbocyclic as defined above.

The term “—C₁₋₆alkylene-(halo)₁₋₃” refers to the —C₁₋₆alkyl as defined above substituted by 1, 2 or 3 halogen as defined above.

The term “heteroC₂₋₆alkyl” refers to the C₂₋₆alkyl as defined above wherein one or more carbon atoms in the chain are replaced by a heteroatom selected from 0, S or N, preferred heteratom is O.

The term “—C₁₋₆alkylene-(OR₈)₁₋₃” refers to the —C₁₋₆alkyl as defined above substituted by 1, 2 or 3 OR₈, wherein R₈ is defined as above, preferred R₈ is selected from hydrogen, methyl, ethyl or propyl.

The term “—C₁₋₆alkylene-(SR₈)₁₋₃” refers to the —C₁₋₆alkyl as defined above substituted by 1, 2 or 3 SR₈, wherein R₈ is defined as above, preferred R₈ is selected from hydrogen, methyl, ethyl or propyl.

The term “—O—C₁₋₆alkylene-(halo)₁₋₃” refers to the oxygen ethers of —C₁₋₆alkylene-(halo)₁₋₃ as defined above.

The term “—S—C₁₋₆alkylene-(halo)₁₋₃” refers to the S ethers of —C₁₋₆alkylene-(halo)₁₋₃ as defined above.

The term “—C₁₋₆alkylene-NR₈R₉” refers to the —C₁₋₆alkyl as defined above substituted by —NR₈R₉, wherein the R₈ and R₉ is defined as above. preferred R₈, R₉ is selected from hydrogen, methyl, ethyl or propyl.

The term “—C₁₋₆alkylene-C(═O)R₈” refers to the —C₁₋₆alkyl as defined above substituted by —C(═O)R₈, wherein the R₈ is defined as above.

The term “—C₁₋₆alkylene-C(═O)OR₈” refers to the —C₁₋₆alkyl as defined above substituted by —C(═O)OR₈, wherein the R₈ is defined as above.

The term “—C₁₋₆alkylene-OC(═O)R₈” refers to the —C₁₋₆alkyl as defined above substituted by —C(═O)R₈, wherein the R₈ is defined as above.

The term “—C₁₋₆alkylene-C(═O)NR₈R₉” refers to the —C₁₋₆alkyl as defined above substituted by —C(═O)NR₈R₉, wherein the R₈ and R₉ is defined as above.

The term “—C₁₋₆alkylene-NR₈C(═O)R₈” refers to the —C₁₋₆alkyl as defined above substituted by —NR₈C(═O)R₈.

The term “—C₁₋₆alkylene-S(═O)₂NR₈R₉” refers to the —C₁₋₆alkyl as defined above substituted by —NR₈C(═O)R₈.

The term “—C₁₋₆alkylene-CN” refers to the —C₁₋₆alkyl as defined above substituted by —CN.

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instant compounds are also part of the present invention. Furthermore, some of the crystalline forms for the compounds may exist as polymorphs and as such are intended to be included in the present invention. In addition, some of the compounds may form solvates with water (i.e., hydrates) or common organic solvents and such solvates are also intended to be encompassed within the scope of this invention.

The compounds of the present invention may also be present in the form of pharmaceutically acceptable salt(s). For use in medicine, the salts of the compounds of this invention refer to non-toxic “pharmaceutically acceptable salt(s)”. The pharmaceutically acceptable salt forms include pharmaceutically acceptable acidic/anionic or basic/cationic salts. The pharmaceutically acceptable acidic/anionic salt generally takes a form in which the basic nitrogen is protonated with an inorganic or organic acid. Representative organic or inorganic acids include hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic. Pharmaceutically acceptable basic/cationic salts include, and are not limited to aluminum, calcium, chloroprocaine, choline, diethanolamine, ethylenediamine, lithium, magnesium, potassium, sodium and zinc.

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including configuration isomers and optical isomers. The configuration isomers include geometric isomers and optical isomers, and optical isomers mainly include enantiomers and diastereomers. The invention includes all possible stereoisomers of the compound.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H(hydrogen), ²H(deuterium) and ³H(tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent.

When a tautomer of the compound exists, the present invention includes any possible tautomers and pharmaceutically acceptable salts thereof, and mixtures thereof, except where specifically stated otherwise.

When the compound and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds are intended for pharmaceutical use they are preferably provided in substantially pure form, for example at least 60% pure, more suitably at least 75% pure, especially at least 98% pure (% are on a weight for weight basis).

The pharmaceutical compositions of the present invention comprise a compound (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds or a prodrug or a metabolite or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt. The compounds or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

### METHODS OF PREPARATION

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

For instance, compounds of the present invention may be prepared according to the General Synthetic Schemes

General Synthetic Schemes:

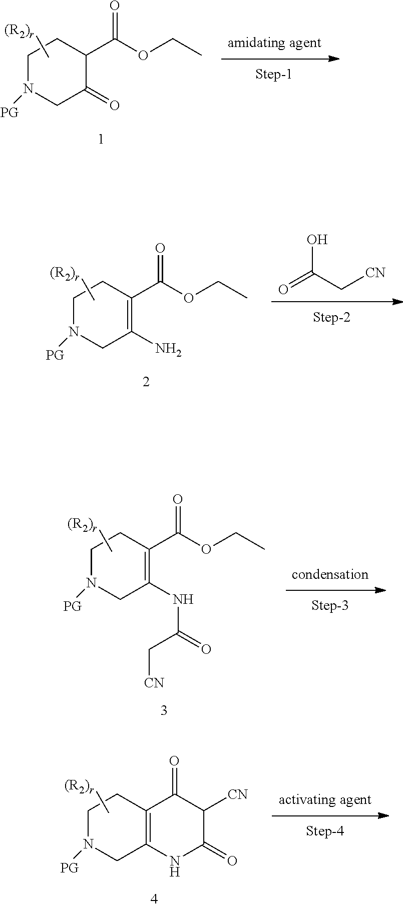

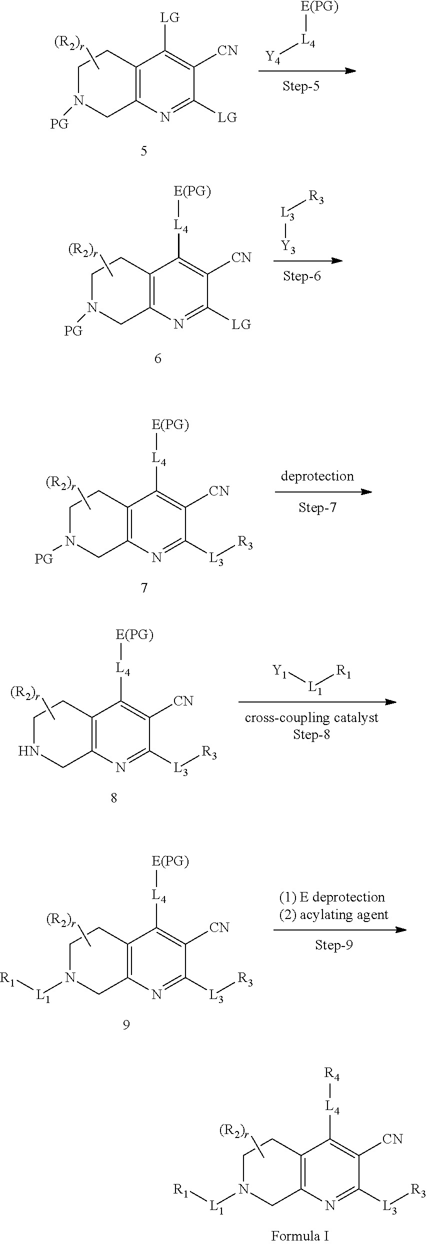

As exemplified in General Synthetic Scheme, a compound such as 1, can be purchased or synthesized (CN104910158) and may be achiral, racemic, or enantiopure. In step-1, compounds such as 1 can be reacted with an amiding reagent such as ammonium hydroxide or ammonium acetate in a polar solvent such as ethanol to form an enamine such as compound 2. In step-2 the enamine can be reacted with 2-cyanoacetic acid to form an amide such as compound 3 by treatment with a condensation reagent such as HATU or EDCl/HOBT, or an acyl chloride. This reaction proceeds in a solvent such as dichloromethane or DMF in the presence of a base such as triethylamine or Hunig's base. In step-3 the amide intermediate cyclized with a cyclization agent to form the pyridinedione ring such as compound 4 in a solvent such as THF. Contemplated cyclization agents include, but are not limited to, bases such as potassium hexamethyldisilazide, potassium tert-butoxide, phosphazene bases, and sodium hydride. In step-4 the oxo groups of the pyridinedione are then converted to leaving groups using an activating agent to form compound 5. Contemplated activating agents include, but not limited to, thionyl chloride, triflic anhydride, phosphorus oxychloride, and phosphorus pentachloride. In step-5 the leaving group is then replaced with a L₄-E(PG) group to form a substituted pyridinone such as compound 6 in a solvent such as acetonitrile and a base such as DIPEA, Each of E at each occurrence is independently selected from

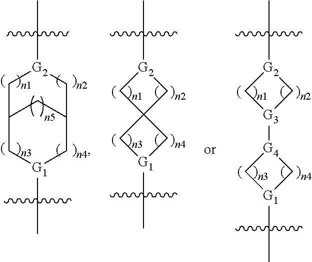

at each occurrence is independently optionally substituted by 1, 2, 3, 4, 5 or 6 R₄₂; In step-6 the L₃-R₃ group can be introduced by substitution of LG to provide compounds such as 7, typically in THF with an appropriate base such as NaH or t-BuONa. At the same time L₃-R₃ could also be introduced by cross-coupling reaction with the appropriate L₃-R₃ reagent, for example in the presence of a palladium catalyst such as Pd₂(dba)₃/BINAP in a solvent such as toluene with a base such as cesium carbonate or sodium tert-butoxide to provide compound 7. In step-8 L₁-R₁ could be introduced by cross-coupling reaction with the appropriate functionalized L₁-R₁ reagent, in the presence of a palladium catalyst such as Pd₂(dba)₃/BINAP in a solvent such as toluene with a base such as cesium carbonate or sodium tert-butoxide to provide compound 9. In step-9 compounds such as 9 can be de-protected by treatment with acid, typically TFA in DCM or HCl in MeOH, to provide the amino compounds, the amino compounds was then acylated to provide compounds such as formula (I), typically by treatment with acryloyl chloride in DCM with TEA or DIPEA as base. In some cases the groups L₁-R₁ and L₃-R₃ may contain protecting groups, which can be removed by an additional step in the synthetic schemes, for example, the PG can be removed with appropriate de-protecting reagent, such as Pd/C/H2 (g), TFA in a solvent such as DCM.

Compounds at every step may be purified by standard techniques such as column chromatography, crystallization, and reverse phase HPLC or Prep-TLC. If necessary, separation of the enantiomers of formula (I) may be carried out under standard methods known in the art such as chiral SFC or HPLC to afford single enantiomers.

### Intermediate A

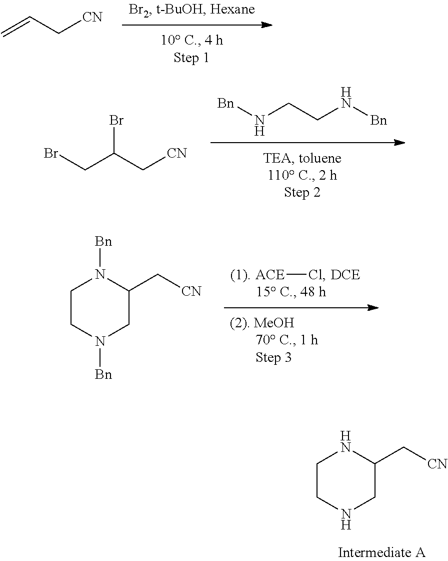

### Intermediate B

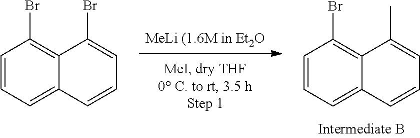

Following procedures of WO2017201161 A1, Intermediate A was prepared from but-3-enenitrile in 3 steps. Intermediate B was prepared from 1,8-dibromonaphthalene in 1 step.

### Example 1

### 4-(4-acryloyl-3-(cyanomethyl)piperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile (Compound 1)

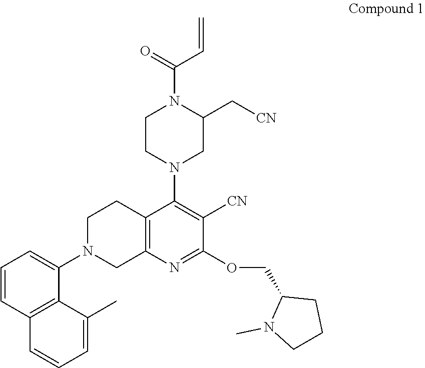 [SMILES: C=CC(=O)N1CCN(c2c(C#N)c(OC[C@@H]3CCCN3C)nc3c2CCN(c2cccc4cccc(C)c24)C3)CC1CC#N]

### Step 1. ethyl 5-amino-1-benzyl-1,2,3,6-tetrahydropyridine-4-carboxylate

Into a 250 mL three neck bottom flask purged and maintained with an inert atmosphere of nitrogen, was placed ethyl 1-benzyl-3-oxopiperidine-4-carboxylate (10.01 g, 38.26 mmol, 1.00 eq), NH₄OAc (58.99 g, 76.53 mmol, 20.00 eq) and EtOH (150 mL). The reaction mixture was stirred at room temperature for 2 hrs. The reaction mixture was filtered and concentrated under vacuum and the resulting residues was partitioned between ethyl acetate and 1 N NaOH aqueous. The aqueous layer was further extracted with dichloromethane (200 mL×2) and the combined organic layers were washed with brine (200 mL), dried over anhydrous sodium sulfate, filtered and concentrated to give a crude product, which was recrystallized with diethyl ether and hexane to gi ye ethyl 5-amino-1-benzyl-1,2,3,6-tetrahydropyridine-4-carboxylate (10.05 g) as off-white solid. LCMS: m/z=261 [M+1]⁺

### Step 2. ethyl 1-benzyl-5-(2-cyanoacetamido)-1,2,3,6-tetrahydropyridine-4-carboxylate

Into a 250-mL round-bottom flask, purged and maintained with an inert atmosphere of nitrogen, was placed 2-cyanoacetic acid (13.14 g, 154.48 mmol, 1.00 eq), DCM (130 mL), followed b y the added of oxalyl chloride (25.49 g, 200.82 mmol, 1.29 eq) in dropwise, and then DMF (0.1 mL) was added. The mixture was stirred for 3 h at 25° C. The resulting solution was concentrated under vacuum. This resulted in 14.01 g (crude product) of 2-cyanoacetylchloride which was used directly in the next step.

Into a 250-mL round-bottom flask purged and maintained with an inert atmosphere of nitrogen, was placed ethyl 5-amino-1-benzyl-1,2,3,6-tetrahydropyridine-4-carboxylate (10.05 g, 38.60 mmol), TEA (23.44 g, 231.64 mmol, 6.00 eq), DCM (100 mL). The mixture was cooled to 0° C. an d then 2-cyanoacetylchloride (14.01 g, crude product) was added in dropwise. The resulting solution was stirred for further 2 hrs at room temperature. The reaction was then quenched by the addition of water (100 mL), extracted with dichloromethane (50 mL×3), the organic layer was combined and washed with brine (50 mL), dried over anhydrous Na₂SO₄, filtered and concentrated under vacuum, the crude product was applied onto a silica gel column eluted with EtOAc/hexane (v/v=3/2). This resulted in 6.02 g (47%, yield) of ethyl 1-benzyl-5-(2-cyanoacetamido)-1,2,3,6-tetrahydropyridine-4-carboxylate as yellow solid. LCMS: m/z=328 [M+1]⁺.

### Step 3. 7-benzyl-2,4-dioxo-1,2,3,4,5,6,7,8-octahydro-1,7-naphthyridine-3-carbonitrile

Into a 100-mL round-bottom flask purged and maintained with an inert atmosphere of nitrogen, was placed ethyl 1-benzyl-5-(2-cyanoacetamido)-1,2,3,6-tetrahydropyridine-4-carboxylate (6.02 g, 18.38 mmol, 1.00 eq), MeONa (2.98 g, 55.16 mmol, 3.00 eq) and MeOH (60 mL). The mixture was stirred for 2 hrs at 65° C. The resulting solution was concentrated under vacuum and the residue was dissolved in 40 mL water and adjusted pH to 6 with AcOH. The solid was collected by filtration, dried under vacuum to provide 3.00 g (58%, yield) of 7-benzyl-2,4-dioxo-1,2,3,4,5,6,7,8-octahydro-1,7-naphthyridine-3-carbonitrile as white solid. LCMS: m/z=282 [M+1]⁺

### Step 4. 7-benzyl-2,4-dichloro-5,6,7,8-tetrahydro-1,7-naphthyridine-3-carbonitrile

Into a 100-mL round-bottom flask purged and maintained with an inert atmosphere of nitrogen, was placed ethyl 1-benzyl-5-(2-cyanoacetamido)-1,2,3,6-tetrahydropyridine-4-carboxylate (3.00 g, 9.16 mmol, 1.00 eq), POCl₃ (30 mL), DIEA (1 mL). The mixture was heated to 80° C. and stir red for 1 h. The solution was concentrated under vacuum. The residue was applied onto a silica gel column eluted with EtOAc/hexane (v/v=3/2). This resulted in 2.10 g (47%, yield) of 7-benzyl-2,4-dichloro-5,6,7,8-tetrahydro-1,7-naphthyridine-3-carbonitrile as yellow oil. LCMS: m/z=31 9 [M+1]⁺

### Step 5. tert-butyl 4-(7-benzyl-2-chloro-3-cyano-5,6,7,8-tetrahydro-1,7-naphthyridin-4-yl)-2-(cyanomethyl)piperazine-1-carboxylate

Into a 100-mL round-bottom flask, was placed 7-benzyl-2,4-dichloro-5,6,7,8-tetrahydro-1,7-naphthyridine-3-carbonitrile (2.10 g, 6.59 mmol, 1.00 eq), 2-(3-methylpiperazin-2-yl)acetonitrile (2.76 g, 19.83 mmol, 3.00 eq), DIEA (3.70 g, 26.30 mmol, 3.99 eq) and DMSO (20 mL), the mixture was stirred at room temperature for 2 hrs. This was followed by the added of Boc₂O (2.87 g, 13.16 mmol, 1.99 eq) in three batches. The mixture was stirred at room temperature overnight. The reaction was then quenched by the addition of water (100 mL), extracted with EtOAc (100 m L×3), the organic layers combined and washed with brine (150 mL), dried over anhydrous Na₂SO₄, filtered and concentrated under vacuum. The residue was purified by silica gel column elute d with EtOAc/hexane (v/v=1/1). This resulted in 1.90 g (57%, yield) of tert-butyl 4-(7-benzyl-2-chloro-3-cyano-5,6,7,8-tetrahydro-1,7-naphthyridin-4-yl)-2-(cyanomethyl)piperazine-1-carboxylate as yellow solid. LCMS: m/z=508 [M+1]⁺

### Step 6. tert-butyl 4-(7-benzyl-3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate

Into a 20-mL sealed tube purged and maintained with an inert atmosphere of nitrogen, was placed tert-butyl 4-(7-benzyl-2-chloro-3-cyano-5,6,7,8-tetrahydro-1,7-naphthyridin-4-yl)-2-(cyanomethyl)piperazine-1-carboxylate (0.45 g, 0.88 mmol, 1.00 eq), Cs₂CO₃ (0.80 g, 2.46 mmol, 2.77 eq), RuPhos Pd G3 (0.18 g, 0.21 mmol, 0.24 eq), (S)-(1-methylpyrrolidin-2-yl)methanol (0.31 g, 2.69 mmol, 3.03 eq) and 1,4-dioxane (10 mL). The mixture was stirred at 90° C. for 1 h. The reaction mixture was concentrated under vacuum. The residue was purified by silica gel column eluted with EtOAc/hexane (v/v=7/3). This resulted in 0.20 g (38%, yield) of tert-butyl 4-(7-benzyl-3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate as yellow solid. LCMS: m/z=586 [M+1]⁺.

### Step 7. tert-butyl 4-(3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate

Into a 50-mL round-bottom flask was placed tert-butyl 4-(7-benzyl-3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate (0.20 g, 0.34 mmol, 1.00 eq), 10% Pd(OH)₂/C (50 mg) and MeOH (10 mL), purged and maintained with an atmosphere of hydrogen, stirred at room temperature for 6 hrs. The catalyst was removed by filtration. The filtrate was concentrated to give tert-butyl 4-(3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate (120 mg, 71%, yield) as a yellow solid. LCMS: m/z=496 [M+1]⁺

### Step 8. tert-butyl 4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-(cyanomethyl)piperazine-1-carboxylate

Into a 20-mL sealed tube purged and maintained with an inert atmosphere of nitrogen, was placed tert-butyl 4-(3-cyano-2-(((S)-1-methylpyrrolidin-2-yl)methoxy)-5,6,7,8-tetrahydro-1,7-naphthyridin-4-yl)-2-(cyanomethyl)piperazine-1-carboxylate (0.12 g, 0.24 mmol, 1.00 eq), Cs₂CO₃ (0.19 g, 0.58 mmol, 2.41 eq), RuPhos Pd G3 (40 mg, 0.05 mmol, 0.21 eq), 1-bromo-8-methylnaphthalene (0.16 g, 0.72 mmol, 3.00 eq) and 1,4-dioxane (6 mL). The mixture was stirred at 100° C. for 3 hrs. The reaction mixture was concentrated under vacuum. The residue was purified by silica gel column eluted with EtOAc/hexane (v/v=3/1). This resulted in 17 mg (11%, yield) of tert-butyl 4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-(cyanomethyl)piperazine-1-carboxylate as yellow solid. LCMS: m/z=636 [M+1]⁺

### Step 9. 4-(4-acryloyl-3-(cyanomethyl)piperazin-1-yl)-7-(8-methylnaphthalen-1-yl)-2-(((S)-1-methyl pyrrolidin-2-yl)methoxy)-5,6,7,8-tetrahydro-1,7-naphthyridine-3-carbonitrile

Into a 50-mL round-bottom flask, was placed tert-butyl 4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-(cyanomethyl)piperazine-1-carboxylate (0.017 g, 26.74 umol), dichloromethane (5 mL), trifluoroacetic acid (3.05 mg, 26.74 umol). The reaction mixture was stirred at room temperature for 45 min. The reaction mixture was concentrated under vacuum. The residue was dissolved in DCM (5 mL), followed by the addition of DIEA (0.02 g, 154.75 umol). The reaction mixture was cooled to 0° C. and acryloyl chloride (0.004 g, 44.19 umol) was added. The reaction mixture was stirred at room temperature for 85 min. After the reaction was completed, the reaction mixture was concentrated under vacuum, the crude product was applied onto a C18 column eluted with CH₃CN/H₂O (0.5% NH₄HCO₃) (v/v=7/3), this afford 5 mg (26.57%, yield) of 4-(4-acryloyl-3-(cyanomethyl)piperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile as brown solid. LCMS: m/z=590M+H⁺

¹H NMR (400 MHz, CD₃OD) δ 7.87-7.63 (m, 2H), 7.59-7.31 (m, 3H), 7.30-7.20 (m, 1H), 6.86 (s, 1H), 6.42-6.26 (m, 1H), 5.95-5.81 (m, 1H), 5.32-5.04 (m, 1H), 4.86-4.71 (m, 1H), 4.62 (s, 1H), 4.53-4.36 (m, 1H), 4.32-4.09 (m, 2H), 4.08-3.41 (m, 8H), 3.30-3.18 (m, 3H), 3.15 (t, J=7.0 Hz, 3H), 2.99 (dd, J=16.8, 6.7 Hz, 1H), 2.90 (s, 3H), 2.48-2.34 (m, 1H), 2.29-1.88 (m, 4H), 1.43-1.22 (m, 1H).

### Example 2

### (S)-4-(4-acryloylpiperazin-1-yl)-7-(8-methylnaphthalen-1-yl)-2-((1-methylpyrrolidin-2-yl)methoxy)-5,6,7,8-tetrahydro-1,7-naphthyridine-3-carbonitrile (Compound 2)

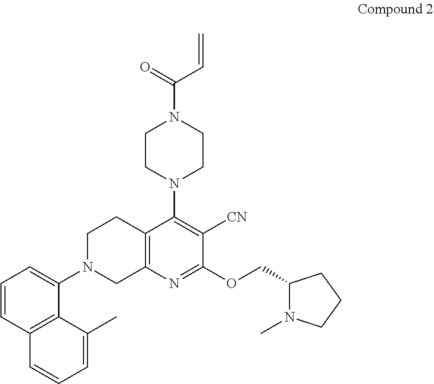 [SMILES: C=CC(=O)N1CCN(c2c(C#N)c(OC[C@@H]3CCCN3C)nc3c2CCN(c2cccc4cccc(C)c24)C3)CC1]

### Step 1. tert-butyl 4-(7-benzyl-2-chloro-3-cyano-5,6,7,8-tetrahydro-1,7-naphthyridin-4-yl)piperazine-1-carboxylate

Into a 50-mL sealed tube purged and maintained with an inert atmosphere of nitrogen, was placed 7-benzyl-2,4-dichloro-5,6,7,8-tetrahydro-1,7-naphthyridine-3-carbonitrile (3.50 g, 11.04 mmol), tert-butyl piperazine-1-carboxylate (4.28 g, 23.01 mmol), DIEA (12.50 g, 96.90 mmol), DMSO (15 mL). The reaction mixture was stirred at 80° C. for 4 hrs. The reaction was then quenched b y the addition of water (20 mL), extracted with Ethyl acetate (50 mL×3), the organic layers w as combined and washed with brine (100 mL), dried over anhydrous Na₂SO₄, filtrated and concentrated under vacuum. The residue was purified by C18 column eluted with ACN/H₂O (v/v=1/1). This resulted in 485 mg of tert-butyl 4-(7-benzyl-2-chloro-3-cyano-5,6,7,8-tetrahydro-1,7-naphthyridin-4-yl)piperazine-1-carboxylate as yellow solid. LCMS: m/z=468 [M+1]⁺

### Step 2. tert-butyl (S)-4-(7-benzyl-3-cyano-2-((1-methylpyrrolidin-2-yl)methoxy)-5,6,7,8-tetrahydro-1,7-naphthyridin-4-yl)piperazine-1-carboxylate

Into a 20-mL sealed tube and maintained with an inert atmosphere of nitrogen, was placed tert-butyl 4-(7-benzyl-2-chloro-3-cyano-5,6,7,8-tetrahydro-1,7-naphthyridin-4-yl)piperazine-1-carboxylate (485 mg, 1.04 mmol), (S)-(1-methylpyrrolidin-2-yl)methanol (413 mg, 3.59 mmol), Ruphos Pd G3 (264 mg, 0.32 mmol), Cs₂CO₃ (1.09 g, 3.33 mmol), 1,4-dioxane (8 mL). The reaction mixture was stirred at 90° C. for 4 hrs. The reaction mixture was filtered and concentrated under vacuum. The residue was purified by C18 column eluted with ACN/H₂O (0.5% NH₄HCO₃) (v/v=3/4). This resulted in 402 mg of tert-butyl (S)-4-(7-benzyl-3-cyano-2-((1-methylpyrrolidin-2-yl)methoxy)-5,6,7,8-tetrahydro-1,7-naphthyridin-4-yl)piperazine-1-carboxylate as yellow solid. LCMS: m/z=54 7 [M+1]⁺

### Step 3. tert-butyl (S)-4-(3-cyano-2-((1-methylpyrrolidin-2-yl)methoxy)-5,6,7,8-tetrahydro-1,7-naphthyridin-4-yl)piperazine-1-carboxylate

Into a 25-mL round-bottom flask was placed tert-butyl (S)-4-(7-benzyl-3-cyano-2-((1-methylpyrrolidin-2-yl)methoxy)-5,6,7,8-tetrahydro-1,7-naphthyridin-4-yl)piperazine-1-carboxylate (402 mg, 0.73 mmol), Pd(OH)₂ (103 mg, 0.74 mmol), EtOAc (20 mL), purged and maintained with an atmosphere of hydrogen. The reaction mixture was stirred at room temperature for 18 hrs. The reaction mixture was filtered and the filtrate was concentrated under vacuum. This resulted in 251 mg (74%) of tert-butyl (S)-4-(3-cyano-2-((1-methylpyrrolidin-2-yl)methoxy)-5,6,7,8-tetrahydro-1,7-naphthyridin-4-yl)piperazine-1-carboxylate as yellow solid. LCMS: m/z=457 [M+1]⁺

### Step 4. tert-butyl (S)-4-(3-cyano-7-(8-methylnaphthalen-1-yl)-2-((1-methylpyrrolidin-2-yl)methoxy)-5,6,7,8-tetrahydro-1,7-naphthyridin-4-yl)piperazine-1-carboxylate

Into a 20-mL sealed tube, purged and maintained with an inert atmosphere of nitrogen, was placed tert-butyl (S)-4-(3-cyano-2-((1-methylpyrrolidin-2-yl)methoxy)-5,6,7,8-tetrahydro-1,7-naphthyridin-4-yl)piperazine-1-carboxylate (251 mg, 0.55 mmol), 1-bromo-8-methylnaphthalene (336 mg, 1.52 mmol), RuPhos Pd G3 (217 mg, 0.26 mmol), Cs₂CO₃ (472 mg, 1.45 mmol), 1,4-dioxane (8 m L). The reaction mixture was stirred at 90° C. for 4 hrs. The reaction mixture was filtered and concentrated under vacuum. The residue was purified by C18 column eluted with ACN/H₂O (v/v=1/1). This resulted in 142 mg (74%) of tert-butyl (S)-4-(3-cyano-7-(8-methylnaphthalen-1-yl)-2-((1-methylpyrrolidin-2-yl)methoxy)-5,6,7,8-tetrahydro-1,7-naphthyridin-4-yl)piperazine-1-carboxylate as yellow oil. LCMS: m/z=597[M+1]⁺

### Step 5. (S)-4-(4-acryloylpiperazin-1-yl)-7-(8-methylnaphthalen-1-yl)-2-((1-methylpyrrolidin-2-yl)methoxy)-5,6,7,8-tetrahydro-1,7-naphthyridine-3-carbonitrile

Into a 50-mL round-bottom flask purged and maintained with an inert atmosphere of nitrogen, was placed tert-butyl (S)-4-(3-cyano-7-(8-methylnaphthalen-1-yl)-2-((1-methylpyrrolidin-2-yl)meth oxy)-5,6,7,8-tetrahydro-1,7-naphthyridin-4-yl)piperazine-1-carboxylate (142 mg, 0.24 mmol), TFA (54 mg, 0.48 mmol), DCM (15 mL). The reaction mixture was stirred at room temperature for 2 hrs. The reaction mixture was concentrated under vacuum. The residue was dissolved by DCM (3 mL) in a 25-mL round-bottom flask, this was followed by the added of DIEA (402 mg, 3.11 mmol). The reaction mixture was cooled to 0° C. and acryloyl chloride (43 mg, 0.48 mmol) was added. The mixture stirred at room temperature for 0.5 h. The reaction was then quenched by the addition of water (10 mL), extracted with DCM (50 mL×3), the organic layers was combined and washed with brine (100 mL), dried over anhydrous Na₂SO₄, filtrated and concentrated un der vacuum. The residue was purified by silica gel column eluted with EtOAc/hexane (v/v=1/1). This resulted in 24 mg (15%) of (S)-4-(4-acryloylpiperazin-1-yl)-7-(8-methylnaphthalen-1-yl)-2-((1-methylpyrrolidin-2-yl)methoxy)-5,6,7,8-tetrahydro-1,7-naphthyridine-3-carbonitrile as yellow solid. LCMS: m/z=551 [M+1]+

¹H NMR (400 MHz, MeOD) δ 8.22 (d, J=8.4 Hz, 1H), 7.99 (d, J=8.3 Hz, 1H), 7.93 (d, J=7.8 Hz, 1H), 7.74 (d, J=6.5 Hz, 1H), 7.56-7.48 (m, 2H), 6.69 (dd, J=16.8, 10.6 Hz, 1H), 6.14 (dd, J=16.8, 1.9 Hz, 1H), 5.68 (dd, J=10.6, 1.9 Hz, 1H), 4.96 (s, 2H), 4.62 (dd, J=12.5, 3.0 Hz, 1H), 4.37-4.21 (m, 3H), 3.88-3.77 (m, 1H), 3.73-3.51 (m, 7H), 3.34 (s, 4H), 3.18-3.07 (m, 1H), 3.06-2.97 (m, 3H), 2.93 (t, J=5.9 Hz, 2H), 2.34-2.22 (m, 1H), 2.15-1.79 (m, 3H), 1.32-1.24 (m, 1H).

### Example 3

### 4-((3S,5R)-4-acryloyl-3,5-dimethylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile (Compound 3)

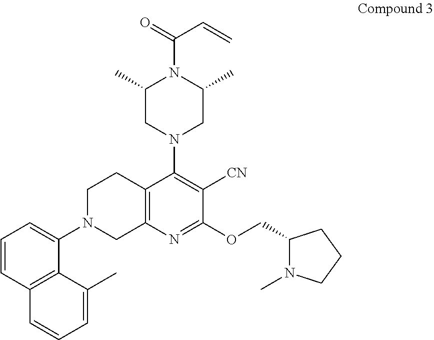 [SMILES: C=CC(=O)N1[C@H](C)CN(c2c(C#N)c(OC[C@@H]3CCCN3C)nc3c2CCN(c2cccc4cccc(C)c24)C3)C[C@@H]1C]

### Step 1. 7-benzyl-2-chloro-4-[(3S,5R)-3,5-dimethylpiperazin-1-yl]-6,8-dihydro-5H-1,7-naphthyridine-3-carbonitrile

Into a 50-mL round-bottom flask purged and maintained with an inert atmosphere of nitrogen, was placed (2R,6S)-2,6-dimethylpiperazine (0.237 g, 2.08 mmol), 7-benzyl-2,4-dichloro-5,6,7,8-tetrahydro-1,7-naphthyridine-3-carbonitrile (0.213 g, 669.39 umol), DIEA (0.373 g, 2.89 mmol), NMP (3 mL), the reaction mixture was stirred for 45 min at 80° C. The reaction mixture was concentrated under vacuum. The crude product was purified by C18 column eluted with ACN/H₂O (v/v=3/1). This afford 230 mg (580.91 umol, 86.78%) of 7-benzyl-2-chloro-4-[(3S,5R)-3,5-dimeth ylpiperazin-1-yl]-6,8-dihydro-5H-1,7-naphthyridine-3-carbonitrile as yellow solid. LCMS: m/z=396 [M+1]⁺.

### Step 2. 7-benzyl-4-[(3S,5R)-3,5-dimethylpiperazin-1-yl]-2-[[(2S)-1-methylpyrrolidin-2-yl]methoxy]-6,8-dihydro-5H-1,7-naphthyridine-3-carbonitrile

Into a 30 mL vial purged and maintained with an inert atmosphere of nitrogen was placed 7-benzyl-2-chloro-4-[(3S,5R)-3,5-dimethylpiperazin-1-yl]-6,8-dihydro-5H-1,7-naphthyridine-3-carbonitrile (0.23 g, 580.91 umol), 1,4-Dioxane (5 mL), (S)-(1-methylpyrrolidin-2-yl)methanol (334.53 mg, 2.90 mmol), RuPhos Pd G3 (48.64 mg, 58.09 umol), Cs₂CO₃ (378.55 mg, 1.16 mmol), the reaction mixture was stirred for 16 hrs at 80° C. The reaction mixture was concentrated under vacuum, and the residue was purified by silica gel column eluted with DCM/MeOH (v/v=3/1). This of ford 220 mg (463.51 umol, 79.79%) of 7-benzyl-4-[(3S,5R)-3,5-dimethylpiperazin-1-yl]-2-[[(2S)-1-m ethylpyrrolidin-2-yl]methoxy]-6,8-dihydro-5H-1,7-naphthyridine-3-carbonitrile as light yellow solid. LCMS: m/z=475 [M+1]⁺.

### Step 3. 4-[(3S,5R)-3,5-dimethylpiperazin-1-yl]-2-[[(2S)-1-methylpyrrolidin-2-yl]methoxy]-5,6,7,8-tetrahydro-1,7-naphthyridine-3-carbonitrile

Into a 50-mL round bottom flask was placed 7-benzyl-4-[(3S,5R)-3,5-dimethylpiperazin-1-yl]-2-[[(2S)-1-methylpyrrolidin-2-yl]methoxy]-6,8-dihydro-5H-1,7-naphthyridine-3-carbonitrile (0.22 g, 463.51 umol), Pd(OH)₂ (0.22 g, 1.57 mmol), ethyl acetate (6 mL), methanol (1 mL), purged and maintained with an atmosphere of hydrogen. the reaction mixture was stirred for 3 hrs at 60° C. The reaction mixture was filtered, the filtrate was evaporated to afford crude product 180 mg of 4-[(3S,5R)-3,5-dimethylpiperazin-1-yl]-2-[[(2S)-1-methylpyrrolidin-2-yl]methoxy]-5,6,7,8-tetrahydro-1,7-naphthyridine-3-carbonitrile as light yellow solid. LCMS: m/z=385 [M+1]⁺.

### Step 4. 4-[(3S,5R)-3,5-dimethylpiperazin-1-yl]-7-(8-methyl-1-naphthyl)-2-[[(2S)-1-methylpyrrolidin-2-yl]methoxy]-6,8-dihydro-5H-1,7-naphthyridine-3-carbonitrile

Into a 30 mL vial purged and maintained with an inert atmosphere of nitrogen, was placed 4-[(3S,5R)-3,5-dimethylpiperazin-1-yl]-2-[[(2S)-1-methylpyrrolidin-2-yl]methoxy]-5,6,7,8-tetrahydro-1,7-naphthyridine-3-carbonitrile (0.18 g, crude product), 1-bromo-8-methylnaphthalene (0.207 g, 936.26 umol), RuPhos Pd G3 (0.04 g, 47.77 umol), Cesium carbonate (0.31 g, 951.45 umol) 1,4-Dioxa ne (5 mL), the reaction mixture was stirred for 16 hrs at 80° C. The reaction mixture was concentrated under vacuum and the residue was purified by silica gel column eluted with EtOAc/Hexane (v/v=7/3). This afford 120 mg (228.70 umol, 48.86% yield) of 4-[(3S,5R)-3,5-dimethylpiperazin-1-yl]-7-(8-methyl-1-naphthyl)-2-[[(2S)-1-methylpyrrolidin-2-yl]methoxy]-6,8-dihydro-5H-1,7-naphthyridine-3-carbonitrile as light yellow solid. LCMS: m/z=525 [M+1]⁺.

### Step 5. 4-((3S,5R)-4-acryloyl-3,5-dimethylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile

Into a 50-mL round-bottom flask purged and maintained with an inert atmosphere of nitrogen, was placed 4-[(3S,5R)-3,5-dimethylpiperazin-1-yl]-7-(8-methyl-1-naphthyl)-2-[[(2S)-1-methylpyrrolidin-2-yl]methoxy]-6,8-dihydro-5H-1,7-naphthyridine-3-carbonitrile (0.12 g, 228.70 umol), DCM (8 mL), triethylamine (0.047 g, 464.48 umol), followed by the addition of acryloyl chloride (0.023 g, 254.12 umol), the reaction mixture was stirred for 80 min at room temperature. The reaction was quenched with H₂O (50 mL), extracted with EtOAc (50 mL×3), the organic layers was combined and washed with brine (50 mL), dried over anhydrous Na₂SO₄, filtrated and concentrated under vacuum. The crude product was purified with HPLC (CH₃CN/H₂O (v/v=7/3), This afford 810 mg (1.40 mmol) of 4-((3S,5R)-4-acryloyl-3,5-dimethylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile as off-white solid. LCMS: m/z=579 [M+1]⁺

¹H NMR (400 MHz, MeOD) δ7.68-7.53 (m, 2H), 7.48-7.31 (m, 2H), 7.29-7.22 (m, 1H), 7.17 (d, J=7.1 Hz, 1H), 6.76 (dd, J=16.7, 10.6 Hz, 1H), 6.20 (dd, J=16.8, 1.9 Hz, 1H), 5.71 (dd, J=10.5, 1.9 Hz, 1H), 4.65-4.35 (m, 3H), 4.32-4.06 (m, 2H), 3.83-3.63 (m, 2H), 3.57-3.47 (m, 1H), 3.44-3.32 (m, 1H), 3.19-2.95 (m, 4H), 2.80 (s, 2H), 2.50-2.38 (m, 1H), 2.19-1.90 (m, 3H), 1.81 (s, 2H), 1.66 (s, 1H), 1.58-1.37 (m, 4H), 1.25-1.18 (m, 6H), 0.83 (t, J=6.8 Hz, 1H).

### Example 4

### (S)-4-((1-acryloylazetidin-3-yl)amino)-7-(8-methylnaphthalen-1-yl)-2-((1-methylpyrrolidin-2-yl)methoxy)-5,6,7,8-tetrahydro-1,7-naphthyridine-3-carbonitrile (Compound 4)

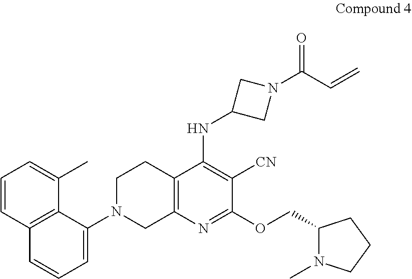 [SMILES: C=CC(=O)N1CC(Nc2c(C#N)c(OC[C@@H]3CCCN3C)nc3c2CCN(c2cccc4cccc(C)c24)C3)C1]

### Step 1. tert-butyl 3-((7-benzyl-2-chloro-3-cyano-5,6,7,8-tetrahydro-1,7-naphthyridin-4-yl)amino)azetidine-1-carboxylate

Into a 50-mL sealed tube purged and maintained with an inert atmosphere of nitrogen, was placed 7-benzyl-2,4-dichloro-5,6,7,8-tetrahydro-1,7-naphthyridine-3-carbonitrile (3.20 g, 10.09 mmol), tert-butyl 3-aminoazetidine-1-carboxylate (4.13 g, 24.03 mmol), DIEA (12.50 g, 96.57 mmol), DMSO (15 mL). The reaction mixture was stirred at 80° C. for 4 hrs. The reaction was then quenched by the addition of water (20 mL), extracted with Ethyl acetate (50 mL×3), the organic layers was combined and washed with brine (100 mL), dried over anhydrous Na₂SO₄, filtrated and concentrated under vacuum. The residue was purified by C18 column eluted with ACN/H₂O (v/v=3/4). This resulted in 560 mg of tert-butyl 3-((7-benzyl-2-chloro-3-cyano-5,6,7,8-tetrahydro-1,7-naphthyridin-4-yl)amino)azetidine-1-carboxylate as yellow solid. LCMS: m/z=454 [M+1]+

### Step 2. tert-butyl (S)-3-((7-benzyl-3-cyano-2-((1-methylpyrrolidin-2-yl)methoxy)-5,6,7,8-tetrahydro-1,7-naphthyridin-4-yl)amino)azetidine-1-carboxylate

Into a 20-mL sealed tube and maintained with an inert atmosphere of nitrogen, was placed t tert-butyl 3-((7-benzyl-2-chloro-3-cyano-5,6,7,8-tetrahydro-1,7-naphthyridin-4-yl)amino)azetidine-1-carboxylate (560 mg, 1.23 mmol), (S)-(1-methylpyrrolidin-2-yl)methanol (527 mg, 4.58 mmol), Ruphos Pd G₃ (213 mg, 0.25 mmol), Cs₂CO₃ (1.08 g, 3.31 mmol), 1,4-dioxane (8 mL). The reaction mixture was stirred at 90° C. for 4 hrs. The reaction mixture was filtered and concentrated under vacuum. The residue was purified by C18 column eluted with ACN/H₂O (0.5%) NH₄HCO₃ (v/v=3/4). This resulted in 590 mg of tert-butyl (S)-3-((7-benzyl-3-cyano-2-((1-methylpyrrolidin-2-yl) methoxy)-5,6,7,8-tetrahydro-1,7-naphthyridin-4-yl)amino)azetidine-1-carboxylate as yellow solid. LCMS: m/z=533 [M+1]⁺

### Step 3. tert-butyl (S)-3-((3-cyano-2-((1-methylpyrrolidin-2-yl)methoxy)-5,6,7,8-tetrahydro-1,7-naphthyridin-4-yl)amino)azetidine-1-carboxylate

Into a 50-mL round-bottom flask was placed tert-butyl (S)-3-((7-benzyl-3-cyano-2-((1-methylpyrrolidin-2-yl)methoxy)-5,6,7,8-tetrahydro-1,7-naphthyridin-4-yl)amino) azetidine-1-carboxylate (590 m g, 1.11 mmol), Pd(OH)₂ (489 mg, 3.48 mmol), MeOH (20 mL), purged and maintained with an atmosphere of hydrogen. The reaction mixture was stirred at room temperature for 8 hrs. The reaction mixture was filtered and the filtrate was concentrated under vacuum. This resulted in 440 mg of tert-butyl (S)-3-((3-cyano-2-((1-methylpyrrolidin-2-yl)methoxy)-5,6,7,8-tetrahydro-1,7-naphthyridin-4-yl)amino)azetidine-1-carboxylate as yellow solid. LCMS: m/z=443 [M+1]⁺

### Step 4. tert-butyl (S)-3-((3-cyano-7-(8-methylnaphthalen-1-yl)-2-((1-methylpyrrolidin-2-yl)methoxy)-5,6,7,8-tetrahydro-1,7-naphthyridin-4-yl)amino)azetidine-1-carboxylate

Into a 20-mL sealed tube purged and maintained with an inert atmosphere of nitrogen, was placed tert-butyl (S)-3-((3-cyano-2-((1-methylpyrrolidin-2-yl)methoxy)-5,6,7,8-tetrahydro-1,7-naphthyridin-4-yl)amino)azetidine-1-carboxylate (440 mg, 0.99 mmol), 1-bromo-8-methylnaphthalene (203 m g, 0.92 mmol), Ruphos Pd G3 (184 mg, 0.22 mmol), Cs₂CO₃ (312 mg, 0.96 mmol), dioxane (8 mL). The reaction mixture was stirred at 90° C. for 4 hrs. The reaction mixture was filtered and concentrated under vacuum. The residue was purified by C18 column eluted with ACN/H₂O (v/v=5/4). This resulted in 145 mg of tert-butyl (S)-3-((3-cyano-7-(8-methylnaphthalen-1-yl)-2-((1-m ethylpyrrolidin-2-yl)methoxy)-5,6,7,8-tetrahydro-1,7-naphthyridin-4-yl)amino) azetidine-1-carboxylate as yellow oil. LCMS: m/z=583 [M+1]⁺.

### Step 5. (S)-4-((1-acryloylazetidin-3-yl)amino)-7-(8-methylnaphthalen-1-yl)-2-((1-methylpyrrolidin-2-yl)methoxy)-5,6,7,8-tetrahydro-1,7-naphthyridine-3-carbonitrile

Into a 50-mL round-bottom flask purged and maintained with an inert atmosphere of nitrogen, was placed tert-butyl (S)-3-((3-cyano-7-(8-methylnaphthalen-1-yl)-2-((1-methylpyrrolidin-2-yl)meth oxy)-5,6,7,8-tetrahydro-1,7-naphthyridin-4-yl)amino)azetidine-1-carboxylate (141 mg, 0.24 mmol), T FA (27 mg, 0.24 mmol), DCM (15 mL). The reaction mixture was stirred at room temperature f or 2 hrs. The reaction mixture was concentrated under vacuum. The residue was dissolved by D CM (3 mL) in a 25-mL round-bottom flask, followed by the addition of DIEA (402 mg, 3.11 m mol). The reaction mixture was cooled to 0° C. and acryloyl chloride (21 mg, 0.23 mmol) was added. The mixture stirred at room temperature for 0.5 h. The reaction was then quenched by the addition of water (20 mL). The resulting solution was extracted with DCM (50 mL×3), the organic layers combined and washed with brine (50 mL), dried over anhydrous Na₂SO₄, filtrated an d concentrated under vacuum. The residue was purified by silica gel column eluted with EtOAc/hexane (v/v=3/4). This resulted in 10 mg (15%) of (S)-4-((1-acryloylazetidin-3-yl)amino)-7-(8-m ethylnaphthalen-1-yl)-2-((1-methylpyrrolidin-2-yl)methoxy)-5,6,7,8-tetrahydro-1,7-naphthyridine-3-carbonitrile as yellow solid. LCMS: m/z=537 [M+H]⁺.

¹H NMR (400 MHz, MeOD) δ 8.34 (d, J=8.9 Hz, 1H), 7.98-7.79 (m, 2H), 7.56-7.42 (m, 3H), 6.46-6.20 (m, 2H), 5.76 (dd, J=10.1, 2.1 Hz, 1H), 4.78-4.59 (m, 1H), 4.54-3.95 (m, 6H), 3.51 (s, 2H), 3.28-2.73 (m, 5H), 2.64 (s, 2H), 2.23-1.82 (m, 4H), 1.81-1.53 (m, 2H), 1.31 (s, 4H).

### Example 5

### 4-((R)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile (Compound 5)

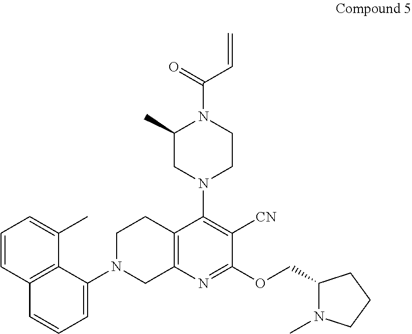 [SMILES: C=CC(=O)N1CCN(c2c(C#N)c(OC[C@@H]3CCCN3C)nc3c2CCN(c2cccc4cccc(C)c24)C3)C[C@H]1C]

### Step 1. tert-butyl (2R)-4-(7-benzyl-2-chloro-3-cyano-6,8-dihydro-5H-1,7-naphthyridin-4-yl)-2-methyl-piperazine-1-carboxylate

Into a 100-mL round-bottom flask was placed 7-benzyl-2,4-dichloro-5,6,7,8-tetrahydro-1,7-naphthyridine-3-carbonitrile (1.05 g, 3.28 mmol), (R)-1-N-Boc-2-methylpiperazine (1 g, 4.99 mmol), NMP (10 mL), N,N-Diisopropylethylamine (1.25 g, 9.64 mmol), the reaction mixture was warmed to 80° C. and stirred for 30 min, another batch of (R)-1-N-Boc-2-methylpiperazine (252 mg) was added. The reaction mixture was stirred further for 80 min. The reaction was quenched with H₂O (50 mL), extracted with EtOAc (50 mL×2), dried over Na₂SO₄, filtrated and concentrated un der vacuum. The residue was purified by silica gel column eluted with EtOAc/hexane (v/v=5/1). The fraction was concentrated to afford tert-butyl (2R)-4-(7-benzyl-2-chloro-3-cyano-6,8-dihydro-5H-1,7-naphthyridin-4-yl)-2-methyl-piperazine-1-carboxylate (541 mg, 1.12 mmol, 34.18% yield) as yellow solid. LCMS: m/z=482 [M+1]+

### Step 2. tert-butyl (2R)-4-[7-benzyl-3-cyano-2-[[(2S)-1-methylpyrrolidin-2-yl]methoxy]-6,8-dihydro-5H-1,7-naphthyridin-4-yl]-2-methyl-piperazine-1-carboxylate

Into a 40-mL vial purged and maintained with an inert atmosphere of nitrogen was placed tert-butyl (2R)-4-(7-benzyl-2-chloro-3-cyano-6,8-dihydro-5H-1,7-naphthyridin-4-yl)-2-methyl-piperazine-1-carboxylate (0.541 g, 1.12 mmol), (S)-(1-methylpyrrolidin-2-yl)methanol (0.526 g, 4.57 mmol), RuPhosPdG3 (0.182 g, 217.35 umol), Cs₂CO₃ (1.08 g, 3.31 mmol), 1,4-Dioxane (0.01 L). The reaction mixture was warmed to 100° C. and stirred for 80 min. The mixture was evaporated under vacuum and applied onto a silica gel column, eluted with EtOAc/hexane (v/v=1/1), to afford ter t-butyl (2R)-4-[7-benzyl-3-cyano-2-[[(2S)-1-methylpyrrolidin-2-yl]methoxy]-6,8-dihydro-5H-1,7-naphthyridin-4-yl]-2-methyl-piperazine-1-carboxylate (511 mg, 911.31 umol, 81.20%). LCMS: m/z=561 [M+1]⁺

### Step 3. tert-butyl (2R)-4-[3-cyano-2-[[(2S)-1-methylpyrrolidin-2-yl]methoxy]-5,6,7,8-tetrahydro-1,7-naphthyridin-4-yl]-2-methyl-piperazine-1-carboxylate

Into a 50-mL 3-neck round bottom flask was placed tert-butyl (2R)-4-[7-benzyl-3-cyano-2-[[(2S)-1-methylpyrrolidin-2-yl]methoxy]-6,8-dihydro-5H-1,7-naphthyridin-4-yl]-2-methyl-piperazine-1-carboxylate (0.511 g, 911.31 umol), EtOAc (10 mL), Pd(OH)₂ (0.522 g, 3.72 mmol), was purged a nd maintained with an atmosphere of hydrogen. The reaction mixture was warmed to 60° C. and stirred for 2 hrs. The reaction mixture was filtered, the filter cake was washed with EtOAc (10 mL×2), the filtrate was concentrated under vacuum to afford tert-butyl (2R)-4-[3-cyano-2-[[(2S)-1-methylpyrrolidin-2-yl]methoxy]-5,6,7,8-tetrahydro-1,7-naphthyridin-4-yl]-2-methyl-piperazine-1-carboxylate (410 mg, 871.22 umol, 95.6% yield) as yellow solid. LCMS: m/z=471 [M+1]⁺

### Step 4. tert-butyl (2R)-4-[3-cyano-7-(8-methyl-1-naphthyl)-2-[[(2S)-1-methylpyrrolidin-2-yl]methoxy]-6,8-dihydro-5H-1,7-naphthyridin-4-yl]-2-methyl-piperazine-1-carboxylate

Into a 40-mL vial purged and maintained with an inert atmosphere of nitrogen was placed tert-butyl (2R)-4-[3-cyano-2-[[(2 S)-1-methylpyrrolidin-2-yl]methoxy]-5,6,7,8-tetrahydro-1,7-naphthyridin-4-yl]-2-methyl-piperazine-1-carboxylate (0.41 g, 871.22 umol), 1-bromo-8-methylnaphthalene (0.427 g, 1.93 mmol), RuPhos Pd G3 (0.213 g, 254.37 umol), Cs₂CO₃ (0.984 g, 3.02 mmol), dioxane (5 mL). The reaction stirred for 16 hrs at 100° C. The mixture was concentrated under vacuum, the residue was purified by silica gel column, eluted with EtOAc/hexane (v/v=3/1). The fraction was concentrated to afford tert-butyl (2R)-4-[3-cyano-7-(8-methyl-1-naphthyl)-2-[[(2S)-1-methy 1pyrrolidin-2-yl]methoxy]-6,8-dihydro-5H-1,7-naphthyridin-4-yl]-2-methyl-piperazine-1-carboxylate (199 mg, 325.81 umol, 37.4% yield) as yellow solid. LCMS: m/z=611 [M+1]⁺

### Step 5. 4-((R)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile

Into a 50-mL round bottom flask was placed tert-butyl (2R)-4-[3-cyano-7-(8-methyl-1-naphthyl)-2-[[(2S)-1-methylpyrrolidin-2-yl]methoxy]-6,8-dihydro-5H-1,7-naphthyridin-4-yl]-2-methyl-piperazine-1-carboxylate (0.199 g, 325.81 umol), DCM (5 mL), TFA (2 mL), the reaction mixture was stirred for 2 hrs at room temperature. After the reaction was completed, the reaction mixture was concentrated under vacuum. The residue was dissolved in dichloromethane (5 mL), this was followed by the added of DIEA (0.118 g, 913.01 umol). Then cooled to 0° C. and acryloyl chloride (0.028 g, 309.36 umol) was added in dropwise. The reaction mixture was stirred for 15 min at room temperature. The reaction was then quenched by the addition of water (20 mL). The resulting solution was extracted with DCM (50 mL×3), the organic layers combined and washed with brine (100 mL), dried over anhydrous Na₂SO₄, filtrated and concentrated under vacuum. The crude product was purified with HPLC eluted with ACN/H₂O (0.5%) NH₄HCO₃ (v/v=1/1). This resulted in (58 mg, 32.72% yield) of 4-((R)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile as yellow solid. LCMS: m/z=565 [M+1]⁺.

¹H NMR (400 MHz, MeOD) δ 7.77-7.63 (m, 2H), 7.49-7.30 (m, 3H), 7.29-7.21 (m, 1H), 6.90-6.75 (m, 1H), 6.26 (dd, J=16.0, 4.3 Hz, 1H), 5.85-5.76 (m, 1H), 4.75 (dd, J=12.5, 3.0 Hz, 1H), 4.46 (dd, J=12.5, 8.3 Hz, 2H), 4.33-4.15 (m, 1H), 4.10-3.89 (m, 2H), 3.88-3.65 (m, 4H), 3.66-3.36 (m, 4H), 3.30-3.11 (m, 6H), 2.96-2.82 (m, 4H), 2.49-2.31 (m, 1H), 2.28-1.91 (m, 3H), 1.50-1.28 (m, 3H).

### Example 6

### 4-((S)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile (Compound 6)

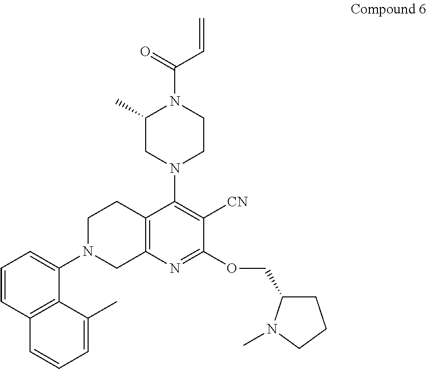 [SMILES: C=CC(=O)N1CCN(c2c(C#N)c(OC[C@@H]3CCCN3C)nc3c2CCN(c2cccc4cccc(C)c24)C3)C[C@@H]1C]

### Step 1. tert-butyl (S)-4-(7-benzyl-2-chloro-3-cyano-5,6,7,8-tetrahydro-1,7-naphthyridin-4-yl)-2-methylpiperazine-1-carboxylate

Into a 25-mL sealed tube purged and maintained with an inert atmosphere of nitrogen, was placed 7-benzyl-2,4-dichloro-5,6,7,8-tetrahydro-1,7-naphthyridine-3-carbonitrile (3.14 g, 9.91 mmol), tert-butyl (S)-2-methylpiperazine-1-carboxylate (4.06 g, 20.27 mmol), DIEA (12.48 g, 96.56 mmol), DMSO (15 mL). The reaction mixture was stirred at 80° C. for 4 hrs. The reaction was then quenched by the addition of water (20 mL), extracted with EtOAc (50 mL×3), the organic lay ers was combined and washed with brine (100 mL), dried over anhydrous Na₂SO₄, filtrated and concentrated under vacuum. The residue was purified by C18 column eluted with ACN/H₂O (v/v=4/5). This resulted in 613 mg of tert-butyl (S)-4-(7-benzyl-2-chloro-3-cyano-5,6,7,8-tetrahydro-1,7-naphthyridin-4-yl)-2-methylpiperazine-1-carboxylate as yellow solid. LCMS: m/z=482 [M+1]⁺

### Step 2. tert-butyl (S)-4-(7-benzyl-3-cyano-2-(((S)-1-methylpyrrolidin-2-yl)methoxy)-5,6,7,8-tetrahydro-1,7-naphthyridin-4-yl)-2-methylpiperazine-1-carboxylate

Into a 30 mL sealed tube purged and maintained with an inert atmosphere of nitrogen, was placed tert-butyl (S)-4-(7-benzyl-2-chloro-3-cyano-5,6,7,8-tetrahydro-1,7-naphthyridin-4-yl)-2-methylpiperazine-1-carboxylate (613 mg, 1.27 mmol), (S)-(1-methylpyrrolidin-2-yl)methanol (468 mg, 4.06 mmol), Ruphos Pd G3 (351 mg, 0.42 mmol), Cs₂CO₃ (1.28 g, 3.92 mmol), 1,4-dioxane (8 mL). The reaction mixture was stirred at 90° C. for 4 hrs. The reaction mixture was filtered and concentrated under vacuum. The residue was purified by C18 column eluted with ACN/H₂O (0.5% N H4HCO₃) (v/v=3/4). This resulted in 364 mg of tert-butyl (S)-4-(7-benzyl-3-cyano-2-(((S)-1-meth ylpyrrolidin-2-yl)methoxy)-5,6,7,8-tetrahydro-1,7-naphthyridin-4-yl)-2-methylpiperazine-1-carboxylate as yellow solid. LCMS: m/z=561 [M+H]⁺

### Step 3. tert-butyl (S)-4-(3-cyano-2-(((S)-1-methylpyrrolidin-2-yl)methoxy)-5,6,7,8-tetrahydro-1,7-naphthyridin-4-yl)-2-methylpiperazine-1-carboxylate

Into a 50 mL round-bottom flask, was placed tert-butyl (S)-4-(7-benzyl-3-cyano-2-(((S)-1-meth ylpyrrolidin-2-yl)methoxy)-5,6,7,8-tetrahydro-1,7-naphthyridin-4-yl)-2-methylpiperazine-1-carboxylate (364 mg, 0.65 mmol), Pd(OH)₂ (723 mg, 5.15 mmol), EtOAc (20 mL), purged and maintained with an atmosphere of hydrogen. The reaction mixture was stirred at room temperature for 8 hrs. The reaction mixture was filtered and the filtrate was concentrated under vacuum. This resulted in 216 mg of tert-butyl (S)-4-(3-cyano-2-(((S)-1-methylpyrrolidin-2-yl)methoxy)-5,6,7,8-tetrahydro-1,7-naphthyridin-4-yl)-2-methylpiperazine-1-carboxylate as yellow solid. LCMS: m/z=471 [M+1]⁺

### Step 4. tert-butyl (S)-4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-methylpiperazine-1-carboxylate

Into a 30 mL sealed tube purged and maintained with an inert atmosphere of nitrogen, was placed tert-butyl (S)-4-(3-cyano-2-(((S)-1-methylpyrrolidin-2-yl)methoxy)-5,6,7,8-tetrahydro-1,7-naphthyridin-4-yl)-2-methylpiperazine-1-carboxylate (216 mg, 0.46 mmol), 1-bromo-8-methylnaphthalene (375 mg, 1.70 mmol), Ruphos Pd G3 (391 mg, 0.47 mmol), Cs₂CO₃ (452 mg, 1.39 mmol), 1,4-dioxane (8 mL). The reaction mixture was stirred at 90° C. for 4 hrs. The reaction mixture was filtered and concentrated under vacuum. The residue was purified by C18 column eluted with A CN/H₂O (v/v=5/4). This resulted in 145 mg of tert-butyl (S)-4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-methylpiperazine-1-carboxylate as yellow oil. LCMS: m/z=611 [M+1]⁺

### Step 5. 4-((S)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile

Into a 50 mL round-bottom flask purged and maintained with an inert atmosphere of nitrogen, was placed tert-butyl (S)-4-(3-cyano-7-(8-methylnaphthalen-1-yl)-2-(((S)-1-methylpyrrolidin-2-yl)methoxy)-5,6,7,8-tetrahydro-1,7-naphthyridin-4-yl)-2-methylpiperazine-1-carboxylate (86 mg, 0.14 mmol), TFA (1.54 g, 13.51 mmol), DCM (10 mL). The reaction mixture was stirred at room temperature for 2 hrs. The reaction mixture was concentrated under vacuum. The residue was dissolved by DCM (3 mL) in 25-mL round-bottom flask. TEA (873 mg, 8.63 mmol) was added. The react ion mixture was cooled to 0° C. and acryloyl chloride (12 mg, 0.14 mmol) was added. The mixture stirred at room temperature for 0.5 h. The reaction was then quenched by the addition of water (50 mL). The resulting solution was extracted with DCM (50 mL×3), the organic layers combined and washed with brine (50 mL), dried over anhydrous Na₂SO₄, filtrated and concentrated under vacuum. The residue was purified by silica gel column eluted with EtOAc/hexane (v/v=3/4). This resulted in 31 mg of 4-((S)-4-acryloyl-3-methylpiperazin-1-yl)-7-(8-methylnaphthalen-1-yl)-2-(((S)-1-methylpyrrolidin-2-yl)methoxy)-5,6,7,8-tetrahydro-1,7-naphthyridine-3-carbonitrile as yellow solid. LCMS: m/z=565 [M+1]⁺

¹H NMR (400 MHz, MeOD) δ 8.33 (d, J=8.4 Hz, 1H), 8.09 (d, J=8.3 Hz, 1H), 8.04 (d, J=7.8 Hz, 1H), 7.82 (d, J=6.6 Hz, 1H), 7.67-7.58 (m, 2H), 6.79 (dd, J=16.7, 10.7 Hz, 1H), 6.25 (dd, J=16.8, 1.7 Hz, 1H), 5.79 (dd, J=10.7, 1.8 Hz, 1H), 5.10-4.94 (m, 2H), 4.85-4.81 (m, 1H), 4.77 (dd, J=12.5, 3.0 Hz, 1H), 4.48-4.37 (m, 2H), 4.33 (s, 2H), 3.97-3.87 (m, 1H), 3.84-3.71 (m, 5H), 3.56-3.38 (m, 3H), 3.31-3.19 (m, 2H), 3.17-3.05 (m, 4H), 3.04-2.91 (m, 1H), 2.54-2.32 (m, 1H), 2.28-1.92 (m, 3H), 1.43-1.25 (m, 3H).

The following examples were synthesized using the above procedure or modification procedure using the corresponding Intermediate.

| Com- | | | MS: |
|---|---|---|---|
| pound | Structure | IUPAC Name | (M + H)⁺ |
| 7 | 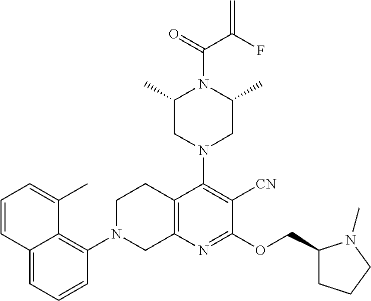 [SMILES: C=C(F)C(=O)N1[C@H](C)CN(c2c(C#N)c(OC[C@@H]3CCCN3C)nc3c2CCN(c2cccc4cccc(C)c24)C3)C[C@@H]1C] | 4-((3R,5S)-4-(2-fluoroacryloyl)-3,5- dimethylpiperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 597 |
| 8 | 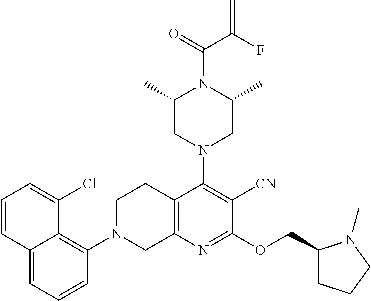 [SMILES: C=C(F)C(=O)N1[C@H](C)CN(c2c(C#N)c(OC[C@@H]3CCCN3C)nc3c2CCN(c2cccc4cccc(Cl)c24)C3)C[C@@H]1C] | 7-(8-chloronaphthalen-1-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 617 |
| 9 | 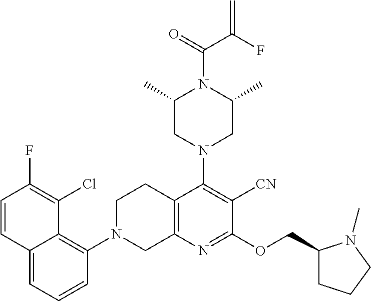 [SMILES: C=C(F)C(=O)N1[C@H](C)CN(c2c(C#N)c(OC[C@@H]3CCCN3C)nc3c2CCN(c2cccc4ccc(F)c(Cl)c24)C3)C[C@@H]1C] | 7-(8-chloro-7-fluoronaphthalen-1-yl)- 4-((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 635 |
| 10 | 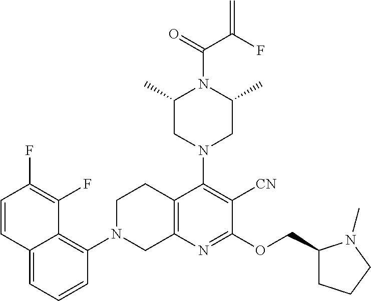 [SMILES: C=C(F)C(=O)N1[C@H](C)CN(c2c(C#N)c(OC[C@@H]3CCCN3C)nc3c2CCN(c2cccc4ccc(F)c(F)c24)C3)C[C@@H]1C] | 7-(7,8-difluoronaphthalen-1-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 619 |
| 11 | 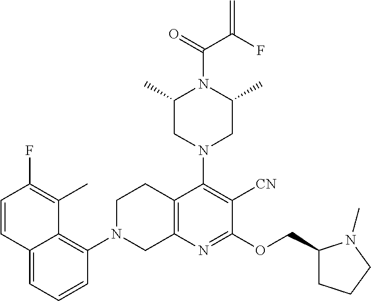 [SMILES: C=C(F)C(=O)N1[C@H](C)CN(c2c(C#N)c(OC[C@@H]3CCCN3C)nc3c2CCN(c2cccc4ccc(F)c(C)c24)C3)C[C@@H]1C] | 7-(7-fluoro-8-methylnaphthalen-1-yl)- 4-((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 615 |
| 12 | 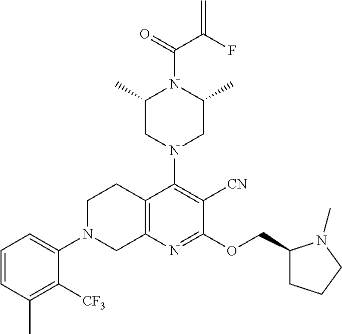 [SMILES: C=C(F)C(=O)N1[C@H](C)CN(c2c(C#N)c(OC[C@@H]3CCCN3C)nc3c2CCN(c2cccc(C)c2C(F)(F)F)C3)C[C@@H]1C] | 4-((3R,5S)-4-(2-fluoroacryloyl)-3,5- dimethylpiperazin-1-yl)-7-(3-methyl- 2-(trifluoromethyl)phenyl)-2-(((S)-1- methylpyrrolidin-2-yl)methoxy)- 5,6,7,8-tetrahydro-1,7-naphthyridine- 3-carbonitrile; | 615 |
| 13 | 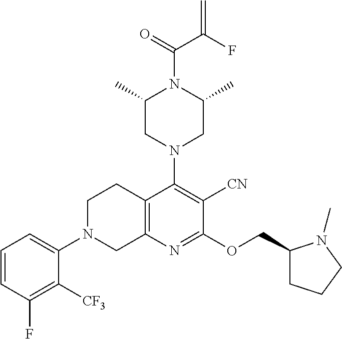 [SMILES: C=C(F)C(=O)N1[C@H](C)CN(c2c(C#N)c(OC[C@@H]3CCCN3C)nc3c2CCN(c2cccc(F)c2C(F)(F)F)C3)C[C@@H]1C] | 7-(3-fluoro-2- (trifluoromethyl)phenyl)-4-((3R,5S)- 4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 619 |
| 14 | 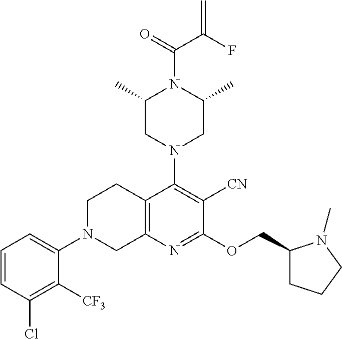 [SMILES: C=C(F)C(=O)N1[C@H](C)CN(c2c(C#N)c(OC[C@@H]3CCCN3C)nc3c2CCN(c2cccc(Cl)c2C(F)(F)F)C3)C[C@@H]1C] | 7-(3-chloro-2- (trifluoromethyl)phenyl)-4-((3R,5S)- 4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 635 |
| 15 | 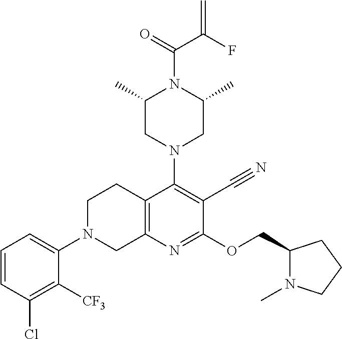 [SMILES: C=C(F)C(=O)N1[C@H](C)CN(c2c(C#N)c(OC[C@H]3CCCN3C)nc3c2CCN(c2cccc(Cl)c2C(F)(F)F)C3)C[C@@H]1C] | 7-(3-chloro-2- (trifluoromethyl)phenyl)-4-((3R,5S)- 4-(2-fluoroacryloyl)-3,5- dimethylpiperazin-1-yl)-2-(((R)-1- methylpyrrolidin-2-yl)methoxy)- 5,6,7,8-tetrahydro-1,7-naphthyridine- 3-carbonitrile; | 635 |
| 16 | 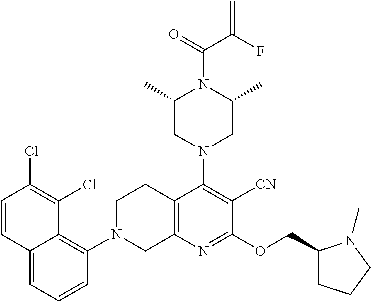 [SMILES: C=C(F)C(=O)N1[C@H](C)CN(c2c(C#N)c(OC[C@@H]3CCCN3C)nc3c2CCN(c2cccc4ccc(Cl)c(Cl)c24)C3)C[C@@H]1C] | 7-(7,8-dichloronaphthalen-1-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 651 |
| 17 | 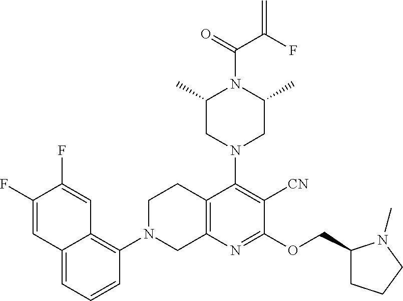 [SMILES: C=C(F)C(=O)N1[C@H](C)CN(c2c(C#N)c(OC[C@@H]3CCCN3C)nc3c2CCN(c2cccc4cc(F)c(F)cc24)C3)C[C@@H]1C] | 7-(6,7-difluoronaphthalen-1-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 619 |
| 18 | 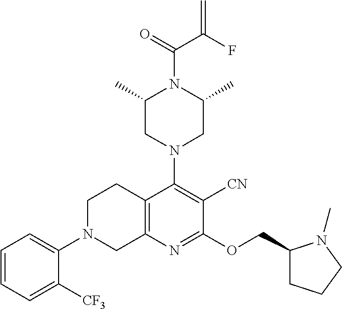 [SMILES: C=C(F)C(=O)N1[C@H](C)CN(c2c(C#N)c(OC[C@@H]3CCCN3C)nc3c2CCN(c2ccccc2C(F)(F)F)C3)C[C@@H]1C] | 4-((3R,5S)-4-(2-fluoroacryloyl)-3,5- dimethylpiperazin-1-yl)-2-(((S)-1- methylpyrrolidin-2-yl)methoxy)-7-(2- (trifluoromethyl)phenyl)-5,6,7,8- tetrahydro-1,7-naphthyridine-3- carbonitrile; | 601 |
| 19 | 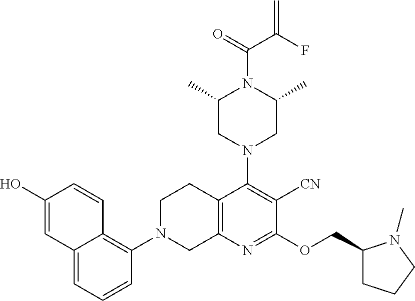 [SMILES: C=C(F)C(=O)N1[C@H](C)CN(c2c(C#N)c(OC[C@@H]3CCCN3C)nc3c2CCN(c2cccc4cc(O)ccc24)C3)C[C@@H]1C] | 4-((3R,5S)-4-(2-fluoroacryloyl)-3,5- dimethylpiperazin-1-yl)-7-(6- hydroxynaphthalen-1-yl)-2-(((S)-1- methylpyrrolidin-2-yl)methoxy)- 5,6,7,8-tetrahydro-1,7-naphthyridine- 3-carbonitrile; | 599 |
| 20 | 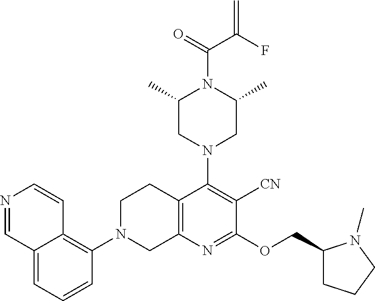 [SMILES: C=C(F)C(=O)N1[C@H](C)CN(c2c(C#N)c(OC[C@@H]3CCCN3C)nc3c2CCN(c2cccc4cnccc24)C3)C[C@@H]1C] | 4-((3R,5S)-4-(2-fluoroacryloyl)-3,5- dimethylpiperazin-1-yl)-7- (isoquinolin-5-yl)-2-(((S)-1- methylpyrrolidin-2-yl)methoxy)- 5,6,7,8-tetrahydro-1,7-naphthyridine- 3-carbonitrile; | 584 |
| 21 | 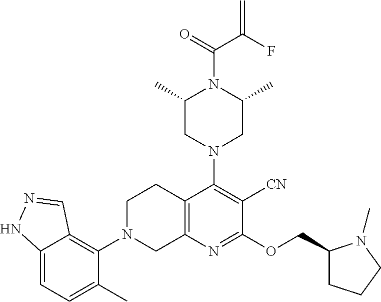 [SMILES: C=C(F)C(=O)N1[C@H](C)CN(c2c(C#N)c(OC[C@@H]3CCCN3C)nc3c2CCN(c2c(C)ccc4[nH]ncc24)C3)C[C@@H]1C] | 4-((3R,5S)-4-(2-fluoroacryloyl)-3,5- dimethylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 587 |
| 22 | 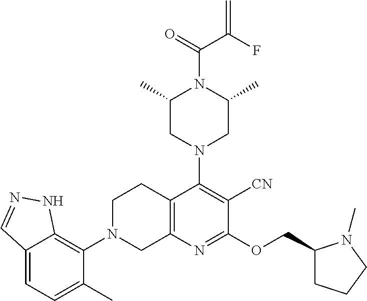 [SMILES: C=C(F)C(=O)N1[C@H](C)CN(c2c(C#N)c(OC[C@@H]3CCCN3C)nc3c2CCN(c2c(C)ccc4cn[nH]c24)C3)C[C@@H]1C] | 4-((3R,5S)-4-(2-fluoroacryloyl)-3,5- dimethylpiperazin-1-yl)-7-(6-methyl- 1H-indazol-7-yl)-2-(((S)-1- methylpyrrolidin-2-yl)methoxy)- 5,6,7,8-tetrahydro-1,7-naphthyridine- 3-carbonitrile; | 587 |
| 23 | 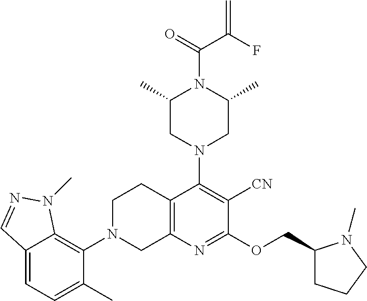 [SMILES: C=C(F)C(=O)N1[C@H](C)CN(c2c(C#N)c(OC[C@@H]3CCCN3C)nc3c2CCN(c2c(C)ccc4cnn(C)c24)C3)C[C@@H]1C] | 7-(1,6-dimethyl-1H-indazol-7-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 601 |
| 24 | 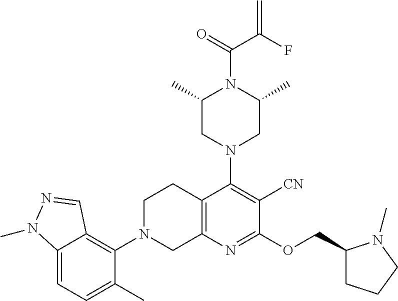 [SMILES: C=C(F)C(=O)N1[C@H](C)CN(c2c(C#N)c(OC[C@@H]3CCCN3C)nc3c2CCN(c2c(C)ccc4c2cnn4C)C3)C[C@@H]1C] | 7-(1,5-dimethyl-1H-indazol-4-yl)-4- ((3R,5S)-4-(2-fluoroacryloyl)-3,5- dimethylpiperazin-1-yl)-2-(((S)-1- methylpyrrolidin-2-yl)methoxy)- 5,6,7,8-tetrahydro-1,7-naphthyridine- 3-carbonitrile; | 601 |
| 25 | 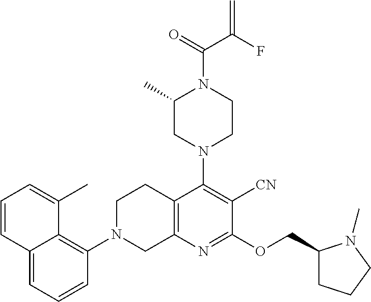 [SMILES: C=C(F)C(=O)N1CCN(c2c(C#N)c(OC[C@@H]3CCCN3C)nc3c2CCN(c2cccc4cccc(C)c24)C3)C[C@@H]1C] | 4-((S)-4-(2-fluoroacryloyl)-3- methylpiperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 583 |
| 26 | 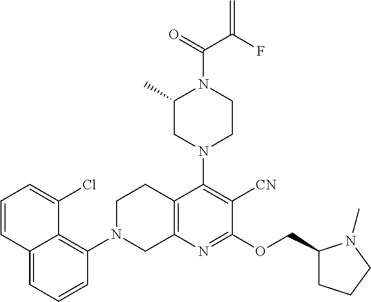 [SMILES: C=C(F)C(=O)N1CCN(c2c(C#N)c(OC[C@@H]3CCCN3C)nc3c2CCN(c2cccc4cccc(Cl)c24)C3)C[C@@H]1C] | 7-(8-chloronaphthalen-1-yl)-4-((S)-4- (2-fluoroacryloyl)-3-methylpiperazin- 1-yl)-2-(((S)-1-methylpyrrolidin-2- yl)methoxy)-5,6,7,8-tetrahydro-1,7- naphthyridine-3-carbonitrile; | 603 |
| 27 | 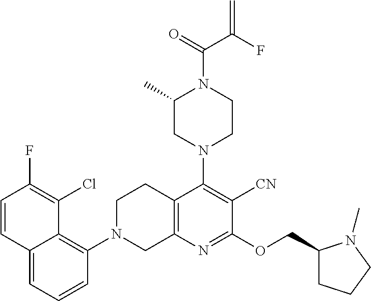 [SMILES: C=C(F)C(=O)N1CCN(c2c(C#N)c(OC[C@@H]3CCCN3C)nc3c2CCN(c2cccc4ccc(F)c(Cl)c24)C3)C[C@@H]1C] | 7-(8-chloro-7-fluoronaphthalen-1-yl)- 4-((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 621 |
| 28 | 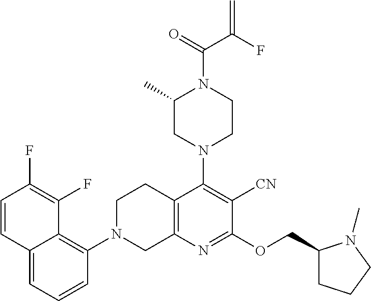 [SMILES: C=C(F)C(=O)N1CCN(c2c(C#N)c(OC[C@@H]3CCCN3C)nc3c2CCN(c2cccc4ccc(F)c(F)c24)C3)C[C@@H]1C] | 7-(7,8-difluoronaphthalen-1-yl)-4- ((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 605 |
| 29 | 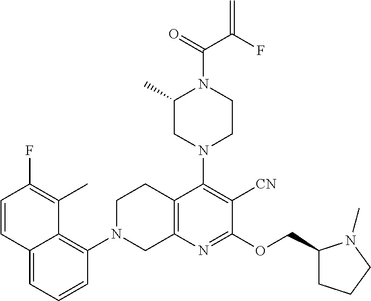 [SMILES: C=C(F)C(=O)N1CCN(c2c(C#N)c(OC[C@@H]3CCCN3C)nc3c2CCN(c2cccc4ccc(F)c(C)c24)C3)C[C@@H]1C] | 7-(7-fluoro-8-methylnaphthalen-1-yl)- 4-((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 601 |
| 30 | 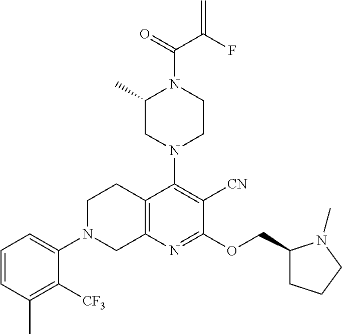 [SMILES: C=C(F)C(=O)N1CCN(c2c(C#N)c(OC[C@@H]3CCCN3C)nc3c2CCN(c2cccc(C)c2C(F)(F)F)C3)C[C@@H]1C] | 4-((S)-4-(2-fluoroacryloyl)-3- methylpiperazin-1-yl)-7-(3-methyl-2- (trifluoromethyl)phenyl)-2-(((S)-1- methylpyrrolidin-2-yl)methoxy)- 5,6,7,8-tetrahydro-1,7-naphthyridine- 3-carbonitrile; | 601 |
| 31 | 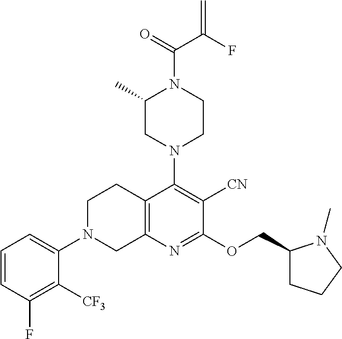 [SMILES: C=C(F)C(=O)N1CCN(c2c(C#N)c(OC[C@@H]3CCCN3C)nc3c2CCN(c2cccc(F)c2C(F)(F)F)C3)C[C@@H]1C] | 7-(3-fluoro-2- (trifluoromethyl)phenyl)-4-((S)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 605 |
| 32 | 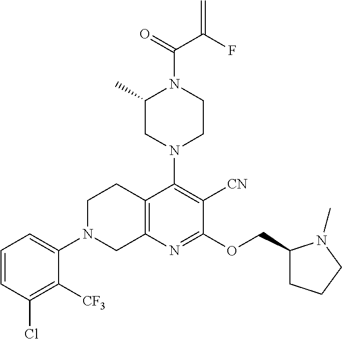 [SMILES: C=C(F)C(=O)N1CCN(c2c(C#N)c(OC[C@@H]3CCCN3C)nc3c2CCN(c2cccc(Cl)c2C(F)(F)F)C3)C[C@@H]1C] | 7-(3-chloro-2- (trifluoromethyl)phenyl)-4-((S)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 621 |
| 33 | 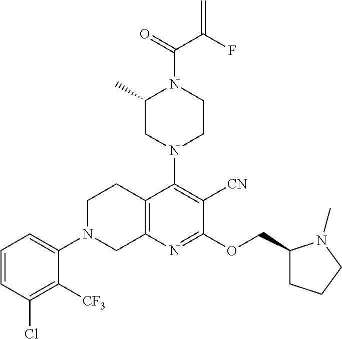 [SMILES: C=C(F)C(=O)N1CCN(c2c(C#N)c(OC[C@@H]3CCCN3C)nc3c2CCN(c2cccc(Cl)c2C(F)(F)F)C3)C[C@@H]1C] | 7-(3-chloro-2- (trifluoromethyl)phenyl)-4-((S)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 621 |
| 34 | 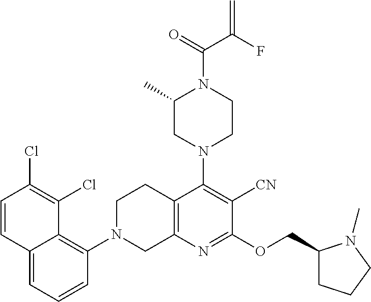 [SMILES: C=C(F)C(=O)N1CCN(c2c(C#N)c(OC[C@@H]3CCCN3C)nc3c2CCN(c2cccc4ccc(Cl)c(Cl)c24)C3)C[C@@H]1C] | 7-(7,8-dichloronaphthalen-1-yl)-4- ((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 637 |
| 35 | 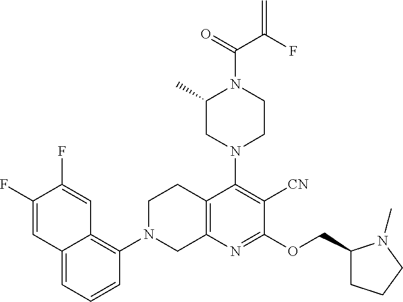 [SMILES: C=C(F)C(=O)N1CCN(c2c(C#N)c(OC[C@@H]3CCCN3C)nc3c2CCN(c2cccc4cc(F)c(F)cc24)C3)C[C@@H]1C] | 7-(6,7-difluoronaphthalen-1-yl)-4- ((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 605 |
| 36 | 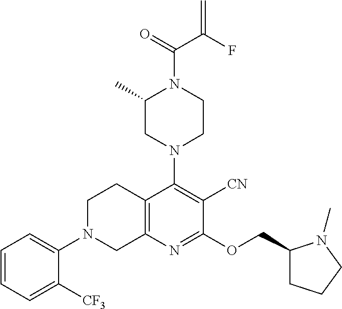 [SMILES: C=C(F)C(=O)N1CCN(c2c(C#N)c(OC[C@@H]3CCCN3C)nc3c2CCN(c2ccccc2C(F)(F)F)C3)C[C@@H]1C] | 4-((S)-4-(2-fluoroacryloyl)-3- methylpiperazin-1-yl)-2-(((S)-1- methylpyrrolidin-2-yl)methoxy)-7-(2- (trifluoromethyl)phenyl)-5,6,7,8- tetrahydro-1,7-naphthyridine-3- carbonitrile; | 587 |
| 37 | 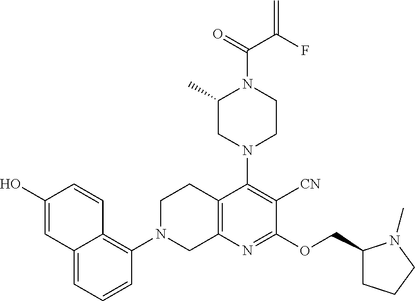 [SMILES: C=C(F)C(=O)N1CCN(c2c(C#N)c(OC[C@@H]3CCCN3C)nc3c2CCN(c2cccc4cc(O)ccc24)C3)C[C@@H]1C] | 4-((S)-4-(2-fluoroacryloyl)-3- methylpiperazin-1-yl)-7-(6- hydroxynaphthalen-1-yl)-2-(((S)-1- methylpyrrolidin-2-yl)methoxy)- 5,6,7,8-tetrahydro-1,7-naphthyridine- 3-carbonitrile; | 585 |
| 38 | 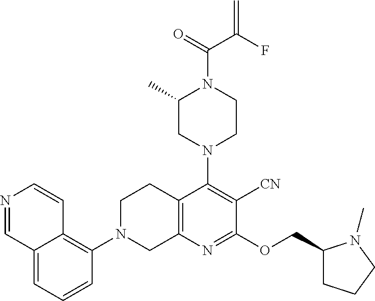 [SMILES: C=C(F)C(=O)N1CCN(c2c(C#N)c(OC[C@@H]3CCCN3C)nc3c2CCN(c2cccc4cnccc24)C3)C[C@@H]1C] | 4-((S)-4-(2-fluoroacryloyl)-3- methylpiperazin-1-yl)-7-(isoquinolin- 5-yl)-2-(((S)-1-methylpyrrolidin-2- yl)methoxy)-5,6,7,8-tetrahydro-1,7- naphthyridine-3-carbonitrile; | 570 |
| 39 | 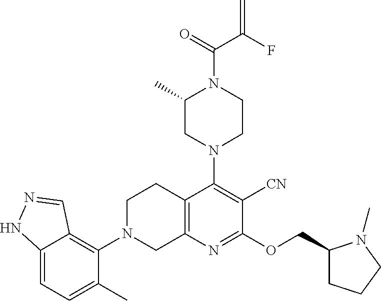 [SMILES: C=C(F)C(=O)N1CCN(c2c(C#N)c(OC[C@@H]3CCCN3C)nc3c2CCN(c2c(C)ccc4[nH]ncc24)C3)C[C@@H]1C] | 4-((S)-4-(2-fluoroacryloyl)-3- methylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 573 |
| 40 | 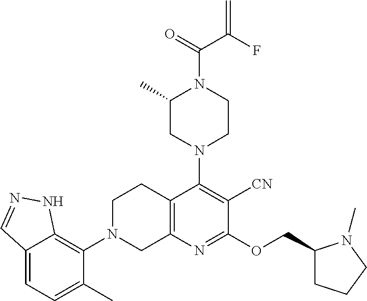 [SMILES: C=C(F)C(=O)N1CCN(c2c(C#N)c(OC[C@@H]3CCCN3C)nc3c2CCN(c2c(C)ccc4cn[nH]c24)C3)C[C@@H]1C] | 4-((S)-4-(2-fluoroacryloyl)-3- methylpiperazin-1-yl)-7-(6-methyl- 1H-indazol-7-yl)-2-(((S)-1- methylpyrrolidin-2-yl)methoxy)- 5,6,7,8-tetrahydro-1,7-naphthyridine- 3-carbonitrile; | 573 |
| 41 | 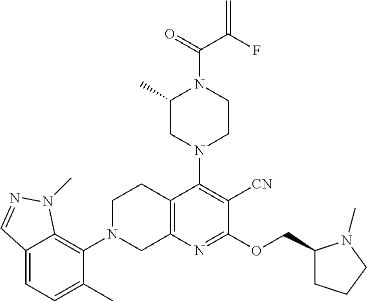 [SMILES: C=C(F)C(=O)N1CCN(c2c(C#N)c(OC[C@@H]3CCCN3C)nc3c2CCN(c2c(C)ccc4cnn(C)c24)C3)C[C@@H]1C] | 7-(1,6-dimethyl-1H-indazol-7-yl)-4- ((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 587 |
| 42 | 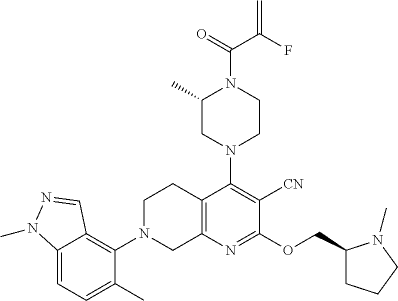 [SMILES: C=C(F)C(=O)N1CCN(c2c(C#N)c(OC[C@@H]3CCCN3C)nc3c2CCN(c2c(C)ccc4c2cnn4C)C3)C[C@@H]1C] | 7-(1,5-dimethyl-1H-indazol-4-yl)-4- ((S)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 587 |
| 43 | 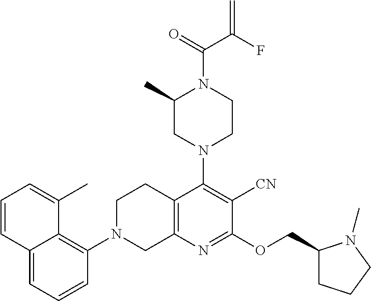 [SMILES: C=C(F)C(=O)N1CCN(c2c(C#N)c(OC[C@@H]3CCCN3C)nc3c2CCN(c2cccc4cccc(C)c24)C3)C[C@H]1C] | 4-((R)-4-(2-fluoroacryloyl)-3- methylpiperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 583 |
| 44 | 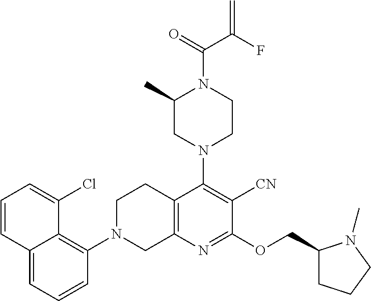 [SMILES: C=C(F)C(=O)N1CCN(c2c(C#N)c(OC[C@@H]3CCCN3C)nc3c2CCN(c2cccc4cccc(Cl)c24)C3)C[C@H]1C] | 7-(8-chloronaphthalen-1-yl)-4-((R)-4- (2-fluoroacryloyl)-3-methylpiperazin- 1-yl)-2-(((S)-1-methylpyrrolidin-2- yl)methoxy)-5,6,7,8-tetrahydro-1,7- naphthyridine-3-carbonitrile; | 603 |
| 45 | 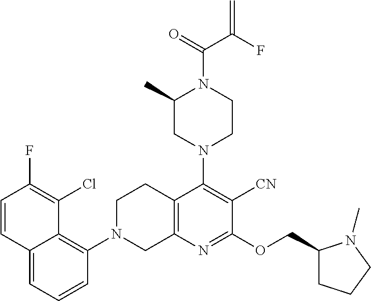 [SMILES: C=C(F)C(=O)N1CCN(c2c(C#N)c(OC[C@@H]3CCCN3C)nc3c2CCN(c2cccc4ccc(F)c(Cl)c24)C3)C[C@H]1C] | 7-(8-chloro-7-fluoronaphthalen-1-yl)- 4-((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 621 |
| 46 | 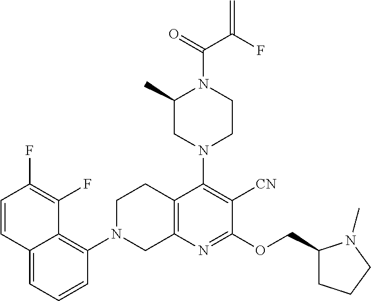 [SMILES: C=C(F)C(=O)N1CCN(c2c(C#N)c(OC[C@@H]3CCCN3C)nc3c2CCN(c2cccc4ccc(F)c(F)c24)C3)C[C@H]1C] | 7-(7,8-difluoronaphthalen-1-yl)-4- ((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 605 |
| 47 | 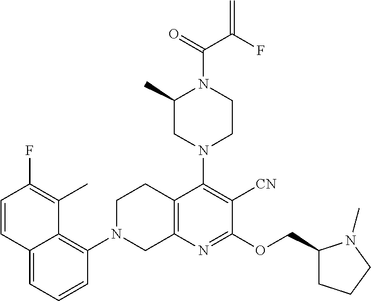 [SMILES: C=C(F)C(=O)N1CCN(c2c(C#N)c(OC[C@@H]3CCCN3C)nc3c2CCN(c2cccc4ccc(F)c(C)c24)C3)C[C@H]1C] | 7-(7-fluoro-8-methylnaphthalen-1-yl)- 4-((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 601 |
| 48 | 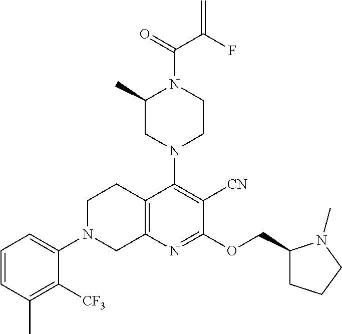 [SMILES: C=C(F)C(=O)N1CCN(c2c(C#N)c(OC[C@@H]3CCCN3C)nc3c2CCN(c2cccc(C)c2C(F)(F)F)C3)C[C@H]1C] | 4-((R)-4-(2-fluoroacryloyl)-3- methylpiperazin-1-yl)-7-(3-methyl-2- (trifluoromethyl)phenyl)-2-(((S)-1- methylpyrrolidin-2-yl)methoxy)- 5,6,7,8-tetrahydro-1,7-naphthyridine- 3-carbonitrile; | 601 |
| 49 | 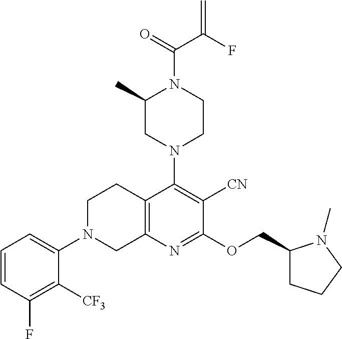 [SMILES: C=C(F)C(=O)N1CCN(c2c(C#N)c(OC[C@@H]3CCCN3C)nc3c2CCN(c2cccc(F)c2C(F)(F)F)C3)C[C@H]1C] | 7-(3-fluoro-2- (trifluoromethyl)phenyl)-4-((R)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 605 |
| 50 | 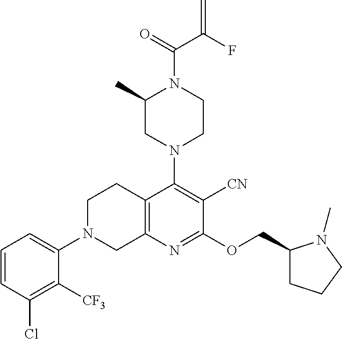 [SMILES: C=C(F)C(=O)N1CCN(c2c(C#N)c(OC[C@@H]3CCCN3C)nc3c2CCN(c2cccc(Cl)c2C(F)(F)F)C3)C[C@H]1C] | 7-(3-chloro-2- (trifluoromethyl)phenyl)-4-((R)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 621 |
| 51 | 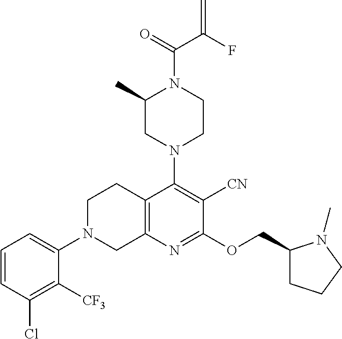 [SMILES: C=C(F)C(=O)N1CCN(c2c(C#N)c(OC[C@@H]3CCCN3C)nc3c2CCN(c2cccc(Cl)c2C(F)(F)F)C3)C[C@H]1C] | 7-(3-chloro-2- (trifluoromethyl)phenyl)-4-((R)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 621 |
| 52 | 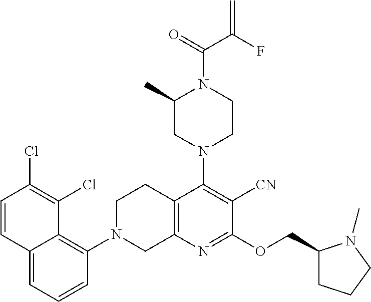 [SMILES: C=C(F)C(=O)N1CCN(c2c(C#N)c(OC[C@@H]3CCCN3C)nc3c2CCN(c2cccc4ccc(Cl)c(Cl)c24)C3)C[C@H]1C] | 7-(7,8-dichloronaphthalen-1-yl)-4- ((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 637 |
| 53 | 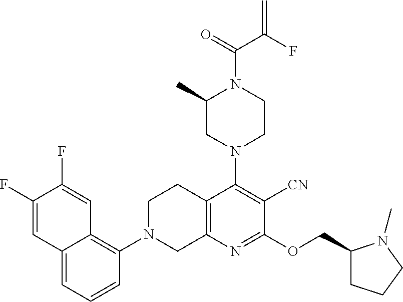 [SMILES: C=C(F)C(=O)N1CCN(c2c(C#N)c(OC[C@@H]3CCCN3C)nc3c2CCN(c2cccc4cc(F)c(F)cc24)C3)C[C@H]1C] | 7-(6,7-difluoronaphthalen-1-yl)-4- ((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 605 |
| 54 | 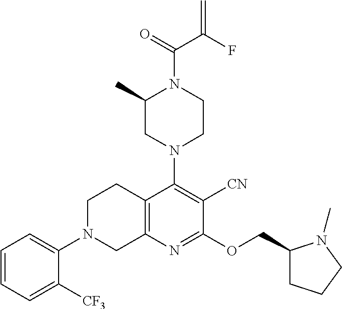 [SMILES: C=C(F)C(=O)N1CCN(c2c(C#N)c(OC[C@@H]3CCCN3C)nc3c2CCN(c2ccccc2C(F)(F)F)C3)C[C@H]1C] | 4-((R)-4-(2-fluoroacryloyl)-3- methylpiperazin-1-yl)-2-(((S)-1- methylpyrrolidin-2-yl)methoxy)-7-(2- (trifluoromethyl)phenyl)-5,6,7,8- tetrahydro-1,7-naphthyridine-3- carbonitrile; | 587 |
| 55 | 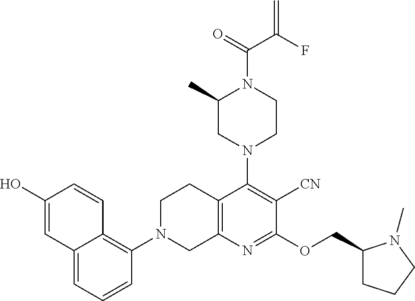 [SMILES: C=C(F)C(=O)N1CCN(c2c(C#N)c(OC[C@@H]3CCCN3C)nc3c2CCN(c2cccc4cc(O)ccc24)C3)C[C@H]1C] | 4-((R)-4-(2-fluoroacryloyl)-3- methylpiperazin-1-yl)-7-(6- hydroxynaphthalen-1-yl)-2-(((S)-1- methylpyrrolidin-2-yl)methoxy)- 5,6,7,8-tetrahydro-1,7-naphthyridine- 3-carbonitrile; | 585 |
| 56 | 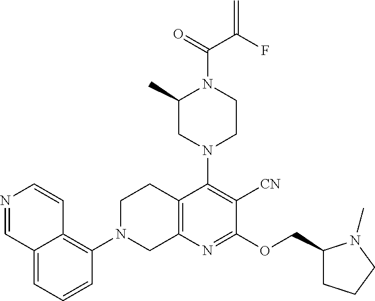 [SMILES: C=C(F)C(=O)N1CCN(c2c(C#N)c(OC[C@@H]3CCCN3C)nc3c2CCN(c2cccc4cnccc24)C3)C[C@H]1C] | 4-((R)-4-(2-fluoroacryloyl)-3- methylpiperazin-1-yl)-7-(isoquinolin- 5-yl)-2-(((S)-1-methylpyrrolidin-2- yl)methoxy)-5,6,7,8-tetrahydro-1,7- naphthyridine-3-carbonitrile; | 570 |
| 57 | 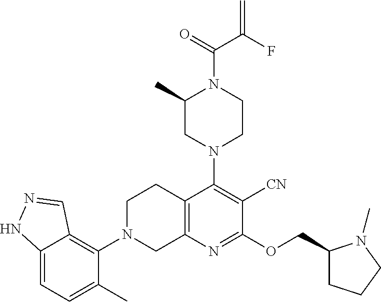 [SMILES: C=C(F)C(=O)N1CCN(c2c(C#N)c(OC[C@@H]3CCCN3C)nc3c2CCN(c2c(C)ccc4[nH]ncc24)C3)C[C@H]1C] | 4-((R)-4-(2-fluoroacryloyl)-3- methylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 573 |
| 58 | 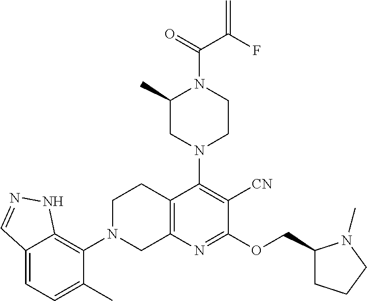 [SMILES: C=C(F)C(=O)N1CCN(c2c(C#N)c(OC[C@@H]3CCCN3C)nc3c2CCN(c2c(C)ccc4cn[nH]c24)C3)C[C@H]1C] | 4-((R)-4-(2-fluoroacryloyl)-3- methylpiperazin-1-yl)-7-(6-methyl- 1H-indazol-7-yl)-2-(((S)-1- methylpyrrolidin-2-yl)methoxy)- 5,6,7,8-tetrahydro-1,7-naphthyridine- 3-carbonitrile; | 573 |
| 59 | 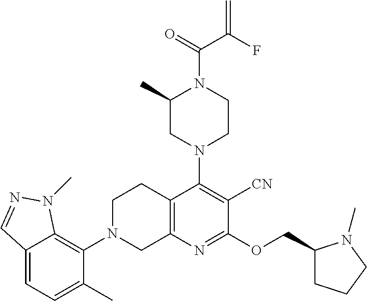 [SMILES: C=C(F)C(=O)N1CCN(c2c(C#N)c(OC[C@@H]3CCCN3C)nc3c2CCN(c2c(C)ccc4cnn(C)c24)C3)C[C@H]1C] | 7-(1,6-dimethyl-1H-indazol-7-yl)-4- ((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 587 |
| 60 | 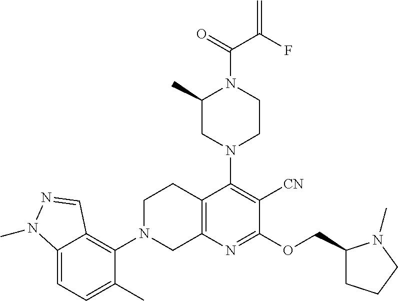 [SMILES: C=C(F)C(=O)N1CCN(c2c(C#N)c(OC[C@@H]3CCCN3C)nc3c2CCN(c2c(C)ccc4c2cnn4C)C3)C[C@H]1C] | 7-(1,5-dimethyl-1H-indazol-4-yl)-4- ((R)-4-(2-fluoroacryloyl)-3- methylpiperazin-1-yl)-2-(((S)-1- methylpyrrolidin-2-yl)methoxy)- 5,6,7,8-tetrahydro-1,7-naphthyridine- 3-carbonitrile; | 587 |
| 61 | 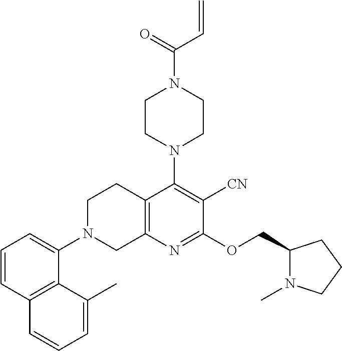 [SMILES: C=CC(=O)N1CCN(c2c(C#N)c(OC[C@H]3CCCN3C)nc3c2CCN(c2cccc4cccc(C)c24)C3)CC1] | (R)-4-(4-acryloylpiperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 551 |
| 62 | 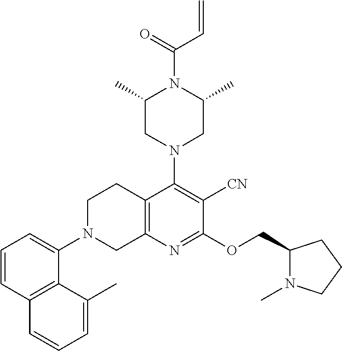 [SMILES: C=CC(=O)N1[C@H](C)CN(c2c(C#N)c(OC[C@H]3CCCN3C)nc3c2CCN(c2cccc4cccc(C)c24)C3)C[C@@H]1C] | 4-((3S,5R)-4-acryloyl-3,5- dimethylpiperazin-1-yl)-7-(8- methylnaphthalen-1-yl)-2-(((R)-1- methylpyrrolidin-2-yl)methoxy)- 5,6,7,8-tetrahydro-1,7-naphthyridine- 3-carbonitrile; | 579 |
| 63 | 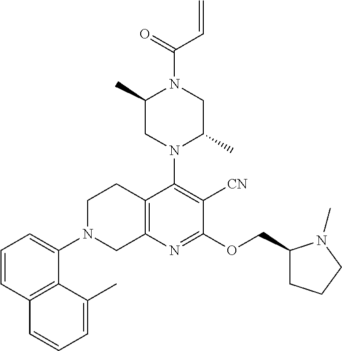 [SMILES: C=CC(=O)N1C[C@H](C)N(c2c(C#N)c(OC[C@@H]3CCCN3C)nc3c2CCN(c2cccc4cccc(C)c24)C3)C[C@H]1C] | 4-((2S,5R)-4-acryloyl-2,5- dimethylpiperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 579 |
| 64 | 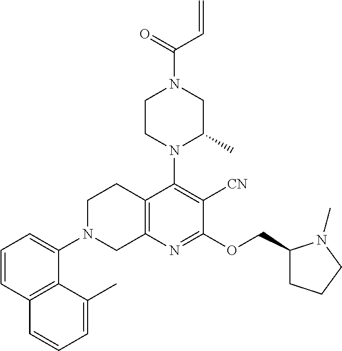 [SMILES: C=CC(=O)N1CCN(c2c(C#N)c(OC[C@@H]3CCCN3C)nc3c2CCN(c2cccc4cccc(C)c24)C3)[C@@H](C)C1] | 4-((S)-4-acryloyl-2-methylpiperazin- 1-yl)-7-(8-methylnaphthalen-1-yl)-2- (((S)-1-methylpyrrolidin-2- yl)methoxy)-5,6,7,8-tetrahydro-1,7- naphthyridine-3-carbonitrile; | 565 |
| 65 | 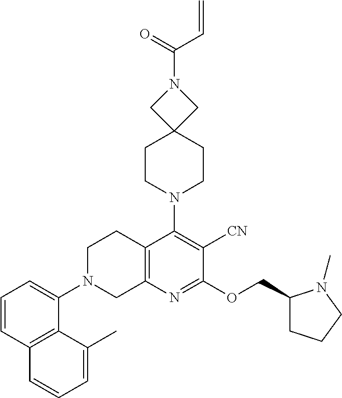 [SMILES: C=CC(=O)N1CC2(CCN(c3c(C#N)c(OC[C@@H]4CCCN4C)nc4c3CCN(c3cccc5cccc(C)c35)C4)CC2)C1] | (S)-4-(2-acryloyl-2,7- diazaspiro[3.5]nonan-7-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 591 |
| 66 | 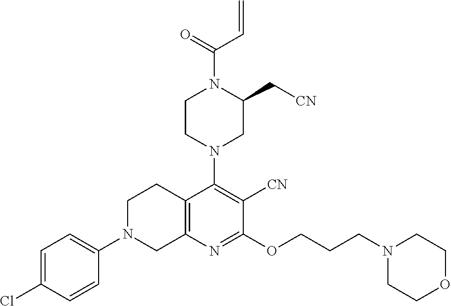 [SMILES: C=CC(=O)N1CCN(c2c(C#N)c(OCCCN3CCOCC3)nc3c2CCN(c2ccc(Cl)cc2)C3)C[C@@H]1CC#N] | (S)-4-(4-acryloyl-3- (cyanomethyl)piperazin-1-yl)-7-(4- chlorophenyl)-2-(3- morpholinopropoxy)-5,6,7,8- tetrahydro-1,7-naphthyridine-3- carbonitrile; | 590 |
| 67 | 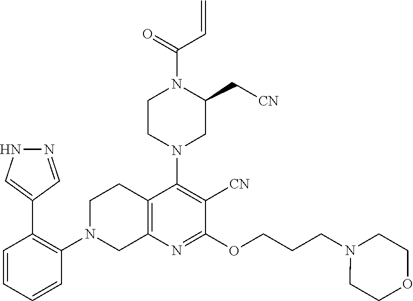 [SMILES: C=CC(=O)N1CCN(c2c(C#N)c(OCCCN3CCOCC3)nc3c2CCN(c2ccccc2-c2cn[nH]c2)C3)C[C@@H]1CC#N] | (S)-7-(2-(1H-pyrazol-4-yl)phenyl)-4- (4-acryloyl-3-(cyanomethyl)piperazin- 1-yl)-2-(3-morpholinopropoxy)- 5,6,7,8-tetrahydro-1,7-naphthyridine- 3-carbonitrile; | 622 |
| 68 | 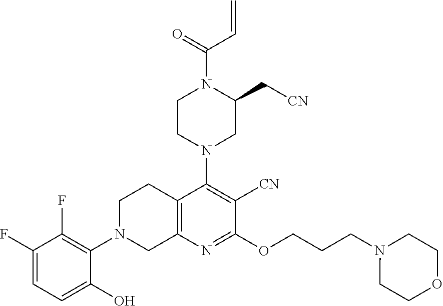 [SMILES: C=CC(=O)N1CCN(c2c(C#N)c(OCCCN3CCOCC3)nc3c2CCN(c2c(O)ccc(F)c2F)C3)C[C@@H]1CC#N] | (S)-4-(4-acryloyl-3- (cyanomethyl)piperazin-1-yl)-7-(2,3- difluoro-6-hydroxyphenyl)-2-(3- morpholinopropoxy)-5,6,7,8- tetrahydro-1,7-naphthyridine-3- carbonitrile; | 608 |
| 69 | 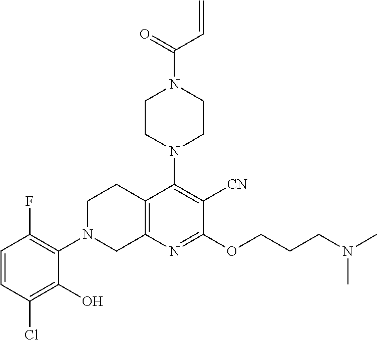 [SMILES: C=CC(=O)N1CCN(c2c(C#N)c(OCCCN(C)C)nc3c2CCN(c2c(F)ccc(Cl)c2O)C3)CC1] | 4-(4-acryloylpiperazin-1-yl)-7-(3- chloro-6-fluoro-2-hydroxyphenyl)-2- (3-(dimethylamino)propoxy)-5,6,7,8- tetrahydro-1,7-naphthyridine-3- carbonitrile; | 543 |
| 70 | 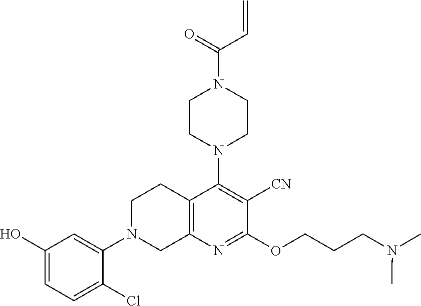 [SMILES: C=CC(=O)N1CCN(c2c(C#N)c(OCCCN(C)C)nc3c2CCN(c2cc(O)ccc2Cl)C3)CC1] | 4-(4-acryloylpiperazin-1-yl)-7-(2- chloro-5-hydroxyphenyl)-2-(3- (dimethylamino)propoxy)-5,6,7,8- tetrahydro-1,7-naphthyridine-3- carbonitrile; | 525 |
| 71 | 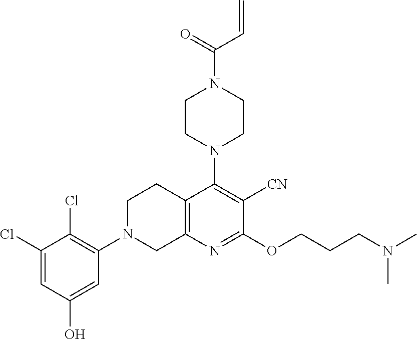 [SMILES: C=CC(=O)N1CCN(c2c(C#N)c(OCCCN(C)C)nc3c2CCN(c2cc(O)cc(Cl)c2Cl)C3)CC1] | 4-(4-acryloylpiperazin-1-yl)-7-(2,3- dichloro-5-hydroxyphenyl)-2-(3- (dimethylamino)propoxy)-5,6,7,8- tetrahydro-1,7-naphthyridine-3- carbonitrile; | 559 |
| 72 | 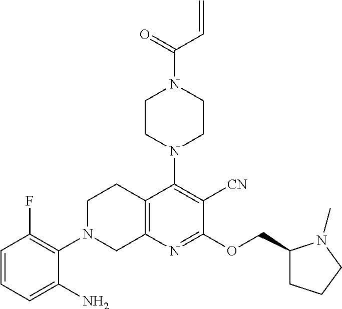 [SMILES: C=CC(=O)N1CCN(c2c(C#N)c(OC[C@@H]3CCCN3C)nc3c2CCN(c2c(N)cccc2F)C3)CC1] | (S)-4-(4-acryloylpiperazin-1-yl)-7-(2- amino-6-fluorophenyl)-2-((1- methylpyrrolidin-2-yl)methoxy)- 5,6,7,8-tetrahydro-1,7-naphthyridine- 3-carbonitrile; | 520 |
| 73 | 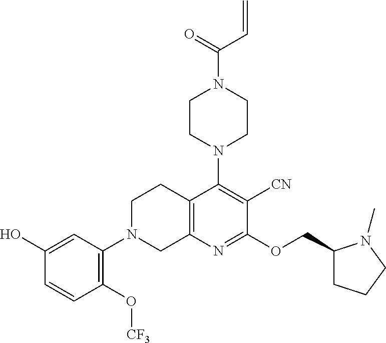 [SMILES: C=CC(=O)N1CCN(c2c(C#N)c(OC[C@@H]3CCCN3C)nc3c2CCN(c2cc(O)ccc2OC(F)(F)F)C3)CC1] | (S)-4-(4-acryloylpiperazin-1-yl)-7-(5- hydroxy-2-(trifluoromethoxy)phenyl)- 2-((1-methylpyrrolidin-2- yl)methoxy)-5,6,7,8-tetrahydro-1,7- naphthyridine-3-carbonitrile; | 587 |
| 74 | 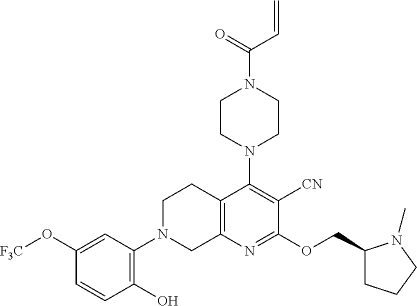 [SMILES: C=CC(=O)N1CCN(c2c(C#N)c(OC[C@@H]3CCCN3C)nc3c2CCN(c2cc(OC(F)(F)F)ccc2O)C3)CC1] | (S)-4-(4-acryloylpiperazin-1-yl)-7-(2- hydroxy-5-(trifluoromethoxy)phenyl)- 2-((1-methylpyrrolidin-2- yl)methoxy)-5,6,7,8-tetrahydro-1,7- naphthyridine-3-carbonitrile; | 587 |
| 75 | 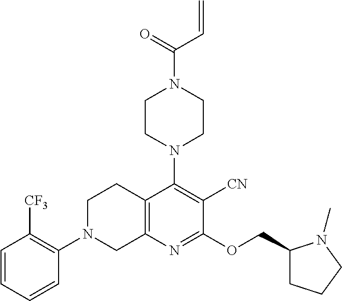 [SMILES: C=CC(=O)N1CCN(c2c(C#N)c(OC[C@@H]3CCCN3C)nc3c2CCN(c2ccccc2C(F)(F)F)C3)CC1] | (S)-4-(4-acryloylpiperazin-1-yl)-2-((1- methylpyrrolidin-2-yl)methoxy)-7-(2- (trifluoromethyl)phenyl)-5,6,7,8- tetrahydro-1,7-naphthyridine-3- carbonitrile; | 555 |
| 76 | 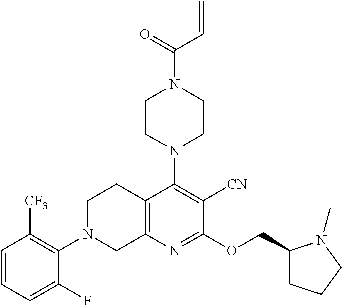 [SMILES: C=CC(=O)N1CCN(c2c(C#N)c(OC[C@@H]3CCCN3C)nc3c2CCN(c2c(F)cccc2C(F)(F)F)C3)CC1] | (S)-4-(4-acryloylpiperazin-1-yl)-7-(2- fluoro-6-(trifluoromethyl)phenyl)-2- ((1-methylpyrrolidin-2-yl)methoxy)- 5,6,7,8-tetrahydro-1,7-naphthyridine- 3-carbonitrile; | 573 |
| 77 | 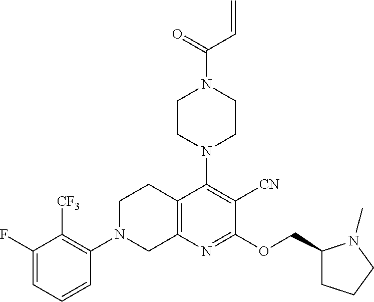 [SMILES: C=CC(=O)N1CCN(c2c(C#N)c(OC[C@@H]3CCCN3C)nc3c2CCN(c2cccc(F)c2C(F)(F)F)C3)CC1] | (S)-4-(4-acryloylpiperazin-1-yl)-7-(3- fluoro-2-(trifluoromethyl)phenyl)-2- ((1-methylpyrrolidin-2-yl)methoxy)- 5,6,7,8-tetrahydro-1,7-naphthyridine- 3-carbonitrile; | 573 |
| 78 | 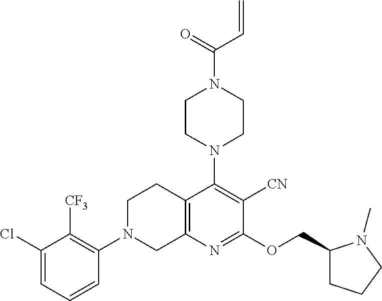 [SMILES: C=CC(=O)N1CCN(c2c(C#N)c(OC[C@@H]3CCCN3C)nc3c2CCN(c2cccc(Cl)c2C(F)(F)F)C3)CC1] | (S)-4-(4-acryloylpiperazin-1-yl)-7-(3- chloro-2-(trifluoromethyl)phenyl)-2- ((1-methylpyrrolidin-2-yl)methoxy)- 5,6,7,8-tetrahydro-1,7-naphthyridine- 3-carbonitrile; | 589 |
| 79 | 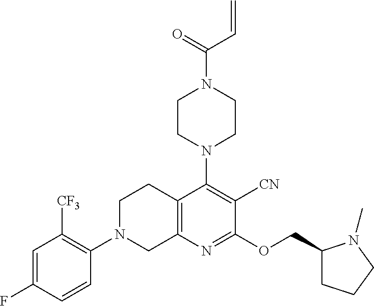 [SMILES: C=CC(=O)N1CCN(c2c(C#N)c(OC[C@@H]3CCCN3C)nc3c2CCN(c2ccc(F)cc2C(F)(F)F)C3)CC1] | (S)-4-(4-acryloylpiperazin-1-yl)-7-(4- fluoro-2-(trifluoromethyl)phenyl)-2- ((1-methylpyrrolidin-2-yl)methoxy)- 5,6,7,8-tetrahydro-1,7-naphthyridine- 3-carbonitrile; | 573 |
| 80 | 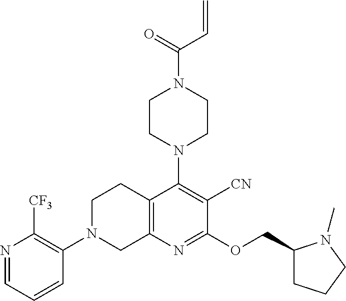 [SMILES: C=CC(=O)N1CCN(c2c(C#N)c(OC[C@@H]3CCCN3C)nc3c2CCN(c2cccnc2C(F)(F)F)C3)CC1] | (S)-4-(4-acryloylpiperazin-1-yl)-2-((1- methylpyrrolidin-2-yl)methoxy)-7-(2- (trifluoromethyl)pyridin-3-yl)-5,6,7,8- tetrahydro-1,7-naphthyridine-3- carbonitrile; | 556 |
| 81 | 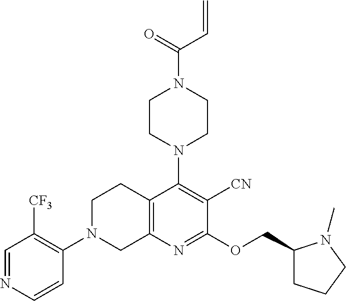 [SMILES: C=CC(=O)N1CCN(c2c(C#N)c(OC[C@@H]3CCCN3C)nc3c2CCN(c2ccncc2C(F)(F)F)C3)CC1] | (S)-4-(4-acryloylpiperazin-1-yl)-2-((1- methylpyrrolidin-2-yl)methoxy)-7-(3- (trifluoromethyl)pyridin-4-yl)-5,6,7,8- tetrahydro-1,7-naphthyridine-3- carbonitrile; | 556 |
| 82 | 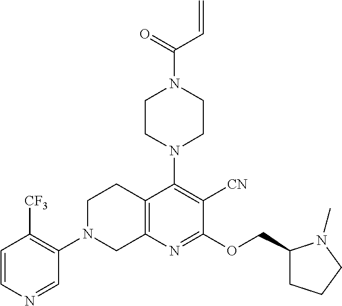 [SMILES: C=CC(=O)N1CCN(c2c(C#N)c(OC[C@@H]3CCCN3C)nc3c2CCN(c2cnccc2C(F)(F)F)C3)CC1] | (S)-4-(4-acryloylpiperazin-1-yl)-2-((1- methylpyrrolidin-2-yl)methoxy)-7-(4- (trifluoromethyl)pyridin-3-yl)-5,6,7,8- tetrahydro-1,7-naphthyridine-3- carbonitrile; | 556 |
| 83 | 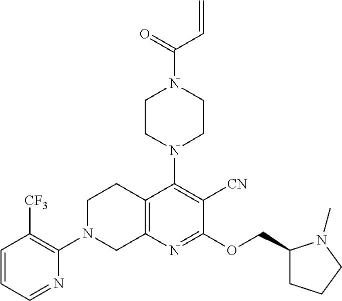 [SMILES: C=CC(=O)N1CCN(c2c(C#N)c(OC[C@@H]3CCCN3C)nc3c2CCN(c2ncccc2C(F)(F)F)C3)CC1] | 4-((3S,5R)-4-acryloyl-3,5- dimethylpiperazin-1-yl)-2-(((S)-1- methylpyrrolidin-2-yl)methoxy)-7-(3- (trifluoromethyl)pyridin-2-yl)-5,6,7,8- tetrahydro-1,7-naphthyridine-3- carbonitrile; | 584 |
| 84 | 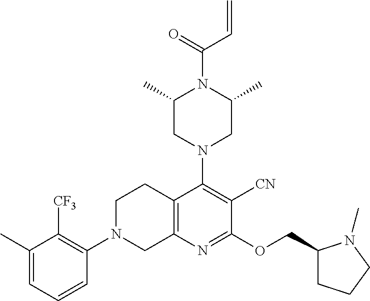 [SMILES: C=CC(=O)N1[C@H](C)CN(c2c(C#N)c(OC[C@@H]3CCCN3C)nc3c2CCN(c2cccc(C)c2C(F)(F)F)C3)C[C@@H]1C] | 4-((3S,5R)-4-acryloyl-3,5- dimethylpiperazin-1-yl)-7-(3-methyl- 2-(trifluoromethyl)phenyl)-2-(((S)-1- methylpyrrolidin-2-yl)methoxy)- 5,6,7,8-tetrahydro-1,7-naphthyridine- 3-carbonitrile; | 597 |
| 85 | 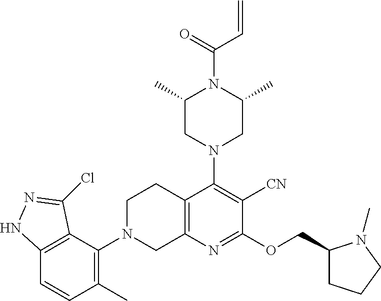 [SMILES: C=CC(=O)N1[C@H](C)CN(c2c(C#N)c(OC[C@@H]3CCCN3C)nc3c2CCN(c2c(C)ccc4[nH]nc(Cl)c24)C3)C[C@@H]1C] | 4-((3S,5R)-4-acryloyl-3,5- dimethylpiperazin-1-yl)-7-(3-chloro- 5-methyl-1H-indazol-4-yl)-2-(((S)-1- methylpyrrolidin-2-yl)methoxy)- 5,6,7,8-tetrahydro-1,7-naphthyridine- 3-carbonitrile; | 603 |
| 86 | 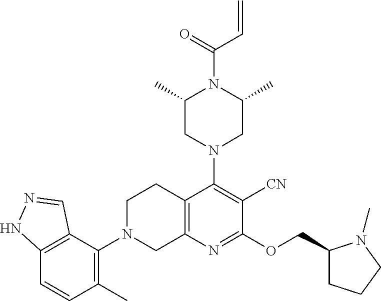 [SMILES: C=CC(=O)N1[C@H](C)CN(c2c(C#N)c(OC[C@@H]3CCCN3C)nc3c2CCN(c2c(C)ccc4[nH]ncc24)C3)C[C@@H]1C] | 4-((3S,5R)-4-acryloyl-3,5- dimethylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 569 |
| 87 | 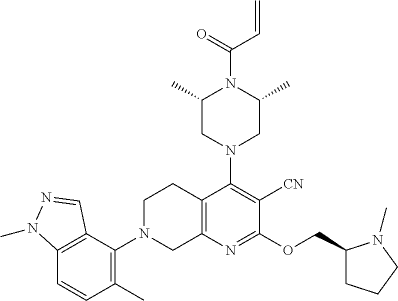 [SMILES: C=CC(=O)N1[C@H](C)CN(c2c(C#N)c(OC[C@@H]3CCCN3C)nc3c2CCN(c2c(C)ccc4c2cnn4C)C3)C[C@@H]1C] | 4-((3S,5R)-4-acryloyl-3,5- dimethylpiperazin-1-yl)-7-(1,5- dimethyl-1H-indazol-4-yl)-2-(((S)-1- methylpyrrolidin-2-yl)methoxy)- 5,6,7,8-tetrahydro-1,7-naphthyridine- 3-carbonitrile; | 583 |
| 88 | 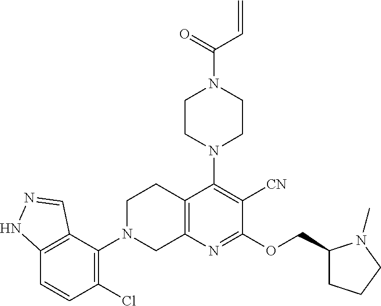 [SMILES: C=CC(=O)N1CCN(c2c(C#N)c(OC[C@@H]3CCCN3C)nc3c2CCN(c2c(Cl)ccc4[nH]ncc24)C3)CC1] | (S)-4-(4-acryloylpiperazin-1-yl)-7-(5- chloro-1H-indazol-4-yl)-2-((1- methylpyrrolidin-2-yl)methoxy)- 5,6,7,8-tetrahydro-1,7-naphthyridine- 3-carbonitrile; | 561 |
| 89 | 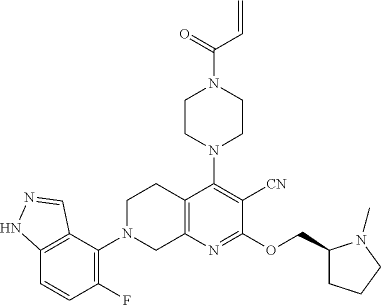 [SMILES: C=CC(=O)N1CCN(c2c(C#N)c(OC[C@@H]3CCCN3C)nc3c2CCN(c2c(F)ccc4[nH]ncc24)C3)CC1] | (S)-4-(4-acryloylpiperazin-1-yl)-7-(5- fluoro-1H-indazol-4-yl)-2-((1- methylpyrrolidin-2-yl)methoxy)- 5,6,7,8-tetrahydro-1,7-naphthyridine- 3-carbonitrile; | 545 |
| 90 | 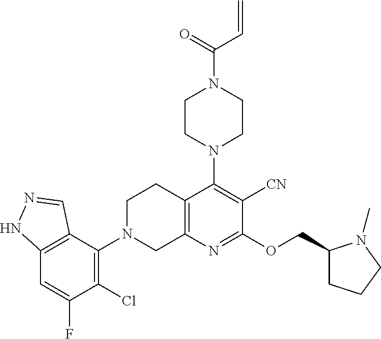 [SMILES: C=CC(=O)N1CCN(c2c(C#N)c(OC[C@@H]3CCCN3C)nc3c2CCN(c2c(Cl)c(F)cc4[nH]ncc24)C3)CC1] | (S)-4-(4-acryloylpiperazin-1-yl)-7-(5- chloro-6-fluoro-1H-indazol-4-yl)-2- ((1-methylpyrrolidin-2-yl)methoxy)- 5,6,7,8-tetrahydro-1,7-naphthyridine- 3-carbonitrile | 579 |
| 91 | 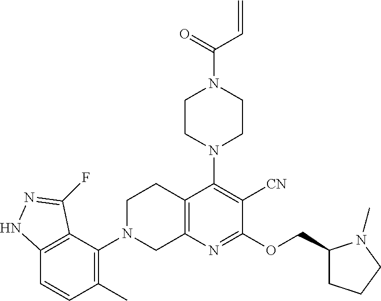 [SMILES: C=CC(=O)N1CCN(c2c(C#N)c(OC[C@@H]3CCCN3C)nc3c2CCN(c2c(C)ccc4[nH]nc(F)c24)C3)CC1] | (S)-4-(4-acryloylpiperazin-1-yl)-7-(3- fluoro-5-methyl-1H-indazol-4-yl)-2- ((1-methylpyrrolidin-2-yl)methoxy)- 5,6,7,8-tetrahydro-1,7-naphthyridine- 3-carbonitrile; | 559 |
| 92 | 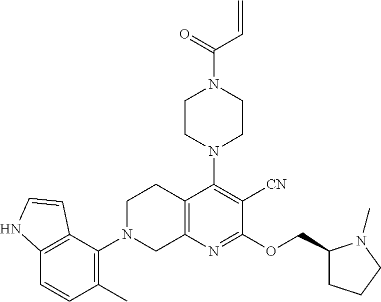 [SMILES: C=CC(=O)N1CCN(c2c(C#N)c(OC[C@@H]3CCCN3C)nc3c2CCN(c2c(C)ccc4[nH]ccc24)C3)CC1] | (S)-4-(4-acryloylpiperazin-1-yl)-7-(5- methyl-1H-indol-4-yl)-2-((1- methylpyrrolidin-2-yl)methoxy)- 5,6,7,8-tetrahydro-1,7-naphthyridine- 3-carbonitrile; | 540 |
| 93 | 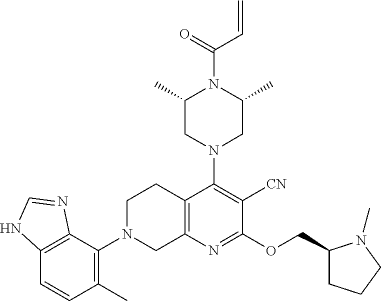 [SMILES: C=CC(=O)N1[C@H](C)CN(c2c(C#N)c(OC[C@@H]3CCCN3C)nc3c2CCN(c2c(C)ccc4[nH]cnc24)C3)C[C@@H]1C] | 4-((3S,5R)-4-acryloyl-3,5- dimethylpiperazin-1-yl)-7-(5-methyl- 1H-benzo[d]imidazol-4-yl)-2-(((S)-1- methylpyrrolidin-2-yl)methoxy)- 5,6,7,8-tetrahydro-1,7-naphthyridine- 3-carbonitrile; | 569 |
| 94 | 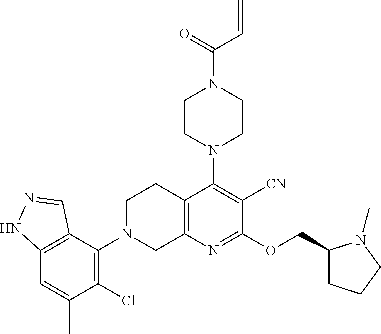 [SMILES: C=CC(=O)N1CCN(c2c(C#N)c(OC[C@@H]3CCCN3C)nc3c2CCN(c2c(Cl)c(C)cc4[nH]ncc24)C3)CC1] | (S)-4-(4-acryloylpiperazin-1-yl)-7-(5- chloro-6-methyl-1H-indazol-4-yl)-2- ((1-methylpyrrolidin-2-yl)methoxy)- 5,6,7,8-tetrahydro-1,7-naphthyridine- 3-carbonitrile; | 575 |
| 95 | 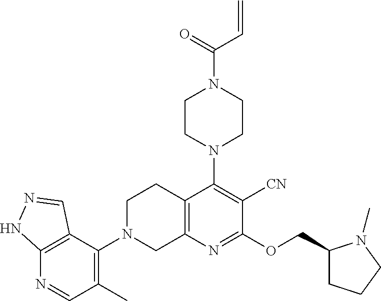 [SMILES: C=CC(=O)N1CCN(c2c(C#N)c(OC[C@@H]3CCCN3C)nc3c2CCN(c2c(C)cnc4[nH]ncc24)C3)CC1] | (S)-4-(4-acryloylpiperazin-1-yl)-7-(5- methyl-1H-pyrazolo[3,4-b]pyridin-4- yl)-2-((1-methylpyrrolidin-2- yl)methoxy)-5,6,7,8-tetrahydro-1,7- naphthyridine-3-carbonitrile; | 542 |
| 96 | 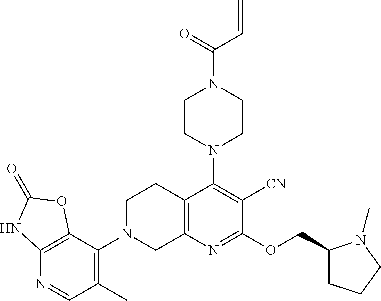 [SMILES: C=CC(=O)N1CCN(c2c(C#N)c(OC[C@@H]3CCCN3C)nc3c2CCN(c2c(C)cnc4[nH]c(=O)oc24)C3)CC1] | (S)-4-(4-acryloylpiperazin-1-yl)-7-(6- methyl-2-oxo-2,3- dihydrooxazolo[4,5-b]pyridin-7-yl)-2- ((1-methylpyrrolidin-2-yl)methoxy)- 5,6,7,8-tetrahydro-1,7-naphthyridine- 3-carbonitrile; | 559 |
| 97 | 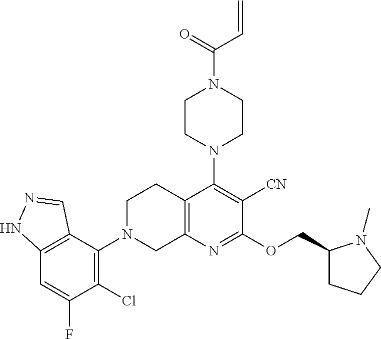 [SMILES: C=CC(=O)N1CCN(c2c(C#N)c(OC[C@@H]3CCCN3C)nc3c2CCN(c2c(Cl)c(F)cc4[nH]ncc24)C3)CC1] | (S)-4-(4-acryloylpiperazin-1-yl)-7-(5- chloro-6-fluoro-1H-indazol-4-yl)-2- ((1-methylpyrrolidin-2-yl)methoxy)- 5,6,7,8-tetrahydro-1,7-naphthyridine- 3-carbonitrile; | 579 |
| 98 | 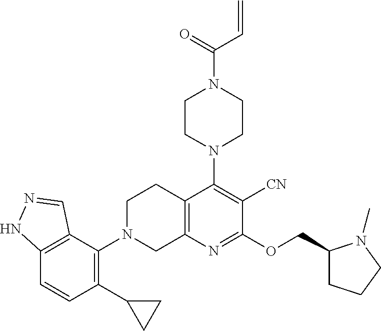 [SMILES: C=CC(=O)N1CCN(c2c(C#N)c(OC[C@@H]3CCCN3C)nc3c2CCN(c2c(C4CC4)ccc4[nH]ncc24)C3)CC1] | (S)-4-(4-acryloylpiperazin-1-yl)-7-(5- cyclopropyl-1H-indazol-4-yl)-2-((1- methylpyrrolidin-2-yl)methoxy)- 5,6,7,8-tetrahydro-1,7-naphthyridine- 3-carbonitrile; | 567 |
| 99 | 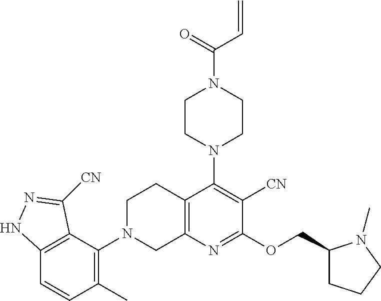 [SMILES: C=CC(=O)N1CCN(c2c(C#N)c(OC[C@@H]3CCCN3C)nc3c2CCN(c2c(C)ccc4[nH]nc(C#N)c24)C3)CC1] | (S)-4-(4-acryloylpiperazin-1-yl)-7-(3- cyano-5-methyl-1H-indazol-4-yl)-2- ((1-methylpyrrolidin-2-yl)methoxy)- 5,6,7,8-tetrahydro-1,7-naphthyridine- 3-carbonitrile; | 566 |
| 100 | 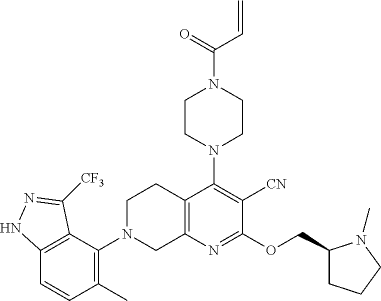 [SMILES: C=CC(=O)N1CCN(c2c(C#N)c(OC[C@@H]3CCCN3C)nc3c2CCN(c2c(C)ccc4[nH]nc(C(F)(F)F)c24)C3)CC1] | (S)-4-(4-acryloylpiperazin-1-yl)-7-(5- methyl-3-(trifluoromethyl)-1H- indazol-4-yl)-2-((1-methylpyrrolidin- 2-yl)methoxy)-5,6,7,8-tetrahydro-1,7- naphthyridine-3-carbonitrile; | 609 |
| 101 | 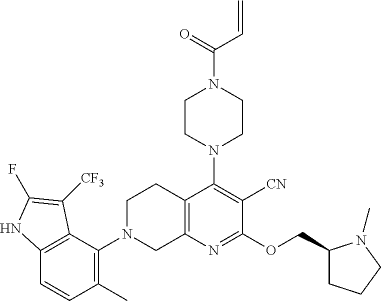 [SMILES: C=CC(=O)N1CCN(c2c(C#N)c(OC[C@@H]3CCCN3C)nc3c2CCN(c2c(C)ccc4[nH]c(F)c(C(F)(F)F)c24)C3)CC1] | (S)-4-(4-acryloylpiperazin-1-yl)-7-(2- fluoro-5-methyl-1H-indol-4-yl)-2-((1- methylpyrrolidin-2-yl)methoxy)- 5,6,7,8-tetrahydro-1,7-naphthyridine- 3-carbonitrile; | 558 |
| 102 | 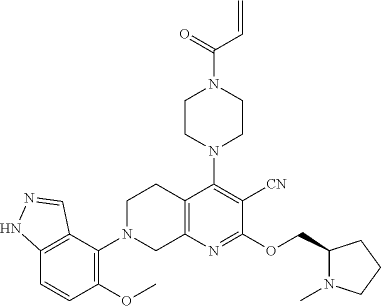 [SMILES: C=CC(=O)N1CCN(c2c(C#N)c(OC[C@H]3CCCN3C)nc3c2CCN(c2c(OC)ccc4[nH]ncc24)C3)CC1] | (R)-4-(4-acryloylpiperazin-1-yl)-7-(5- methoxy-1H-indazol-4-yl)-2-((1- methylpyrrolidin-2-yl)methoxy)- 5,6,7,8-tetrahydro-1,7-naphthyridine- 3-carbonitrile; | 557 |
| 103 | 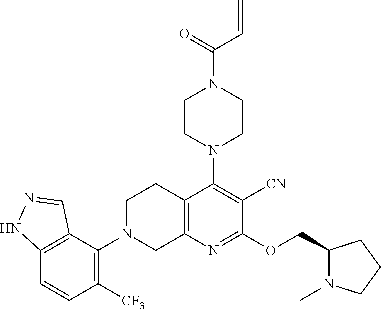 [SMILES: C=CC(=O)N1CCN(c2c(C#N)c(OC[C@H]3CCCN3C)nc3c2CCN(c2c(C(F)(F)F)ccc4[nH]ncc24)C3)CC1] | (R)-4-(4-acryloylpiperazin-1-yl)-2- ((1-methylpyrrolidin-2-yl)methoxy)- 7-(5-(trifluoromethyl)-1H-indazol-4- yl)-5,6,7,8-tetrahydro-1,7- naphthyridine-3-carbonitrile; | 595 |
| 104 | 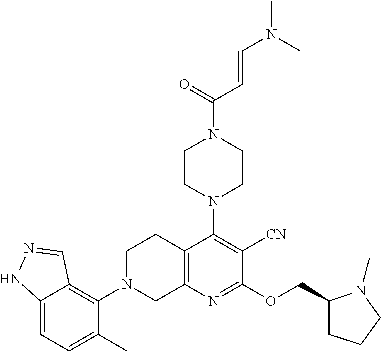 [SMILES: Cc1ccc2[nH]ncc2c1N1CCc2c(nc(OC[C@@H]3CCCN3C)c(C#N)c2N2CCN(C(=O)/C=C/N(C)C)CC2)C1] | (S,E)-4-(4-(3- (dimethylamino)acryloyl)piperazin-1- yl)-7-(5-methyl-1H-indazol-4-yl)-2- ((1-methylpyrrolidin-2-yl)methoxy)- 5,6,7,8-tetrahydro-1,7-naphthyridine- 3-carbonitrile; | 584 |
| 105 | 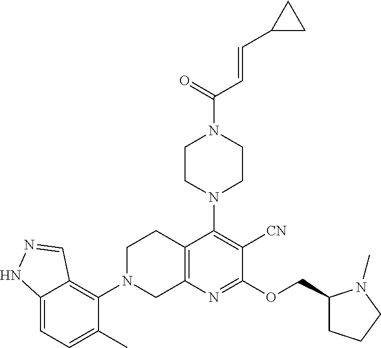 [SMILES: Cc1ccc2[nH]ncc2c1N1CCc2c(nc(OC[C@@H]3CCCN3C)c(C#N)c2N2CCN(C(=O)/C=C/C3CC3)CC2)C1] | (S,E)-4-(4-(3- cyclopropylacryloyl)piperazin-1-yl)- 7-(5-methyl-1H-indazol-4-yl)-2-((1- methylpyrrolidin-2-yl)methoxy)- 5,6,7,8-tetrahydro-1,7-naphthyridine- 3-carbonitrile; | 581 |
| 106 | 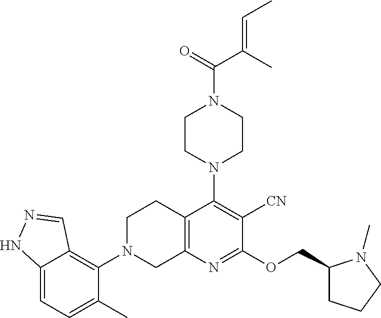 [SMILES: C/C=C(\C)C(=O)N1CCN(c2c(C#N)c(OC[C@@H]3CCCN3C)nc3c2CCN(c2c(C)ccc4[nH]ncc24)C3)CC1] | (S,E)-7-(5-methyl-1H-indazol-4-yl)-4- (4-(2-methylbut-2-enoyl)piperazin-1- yl)-2-((1-methylpyrrolidin-2- yl)methoxy)-5,6,7,8-tetrahydro-1,7- naphthyridine-3-carbonitrile; | 569 |
| 107 | 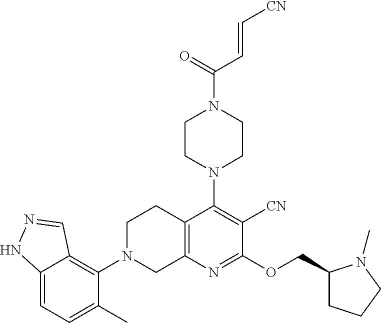 [SMILES: Cc1ccc2[nH]ncc2c1N1CCc2c(nc(OC[C@@H]3CCCN3C)c(C#N)c2N2CCN(C(=O)/C=C/C#N)CC2)C1] | (S,E)-4-(4-(3- cyanoacryloyl)piperazin-1-yl)-7-(5- methyl-1H-indazol-4-yl)-2-((1- methylpyrrolidin-2-yl)methoxy)- 5,6,7,8-tetrahydro-1,7-naphthyridine- 3-carbonitrile; | 566 |
| 108 | 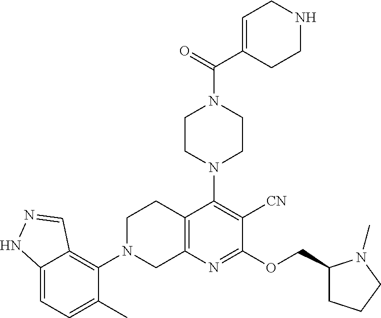 [SMILES: Cc1ccc2[nH]ncc2c1N1CCc2c(nc(OC[C@@H]3CCCN3C)c(C#N)c2N2CCN(C(=O)C3=CCNCC3)CC2)C1] | (S)-7-(5-methyl-1H-indazol-4-yl)-2- ((1-methylpyrrolidin-2-yl)methoxy)- 4-(4-(1,2,3,6-tetrahydropyridine-4- carbonyl)piperazin-1-yl)-5,6,7,8- tetrahydro-1,7-naphthyridine-3- carbonitrile; | 596 |
| 109 | 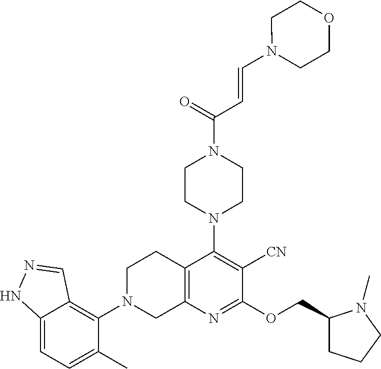 [SMILES: Cc1ccc2[nH]ncc2c1N1CCc2c(nc(OC[C@@H]3CCCN3C)c(C#N)c2N2CCN(C(=O)/C=C/N3CCOCC3)CC2)C1] | (S,E)-7-(5-methyl-1H-indazol-4-yl)-2- ((1-methylpyrrolidin-2-yl)methoxy)- 4-(4-(3-morpholinoacryloyl)piperazin- 1-yl)-5,6,7,8-tetrahydro-1,7- naphthyridine-3-carbonitrile; | 626 |
| 110 | 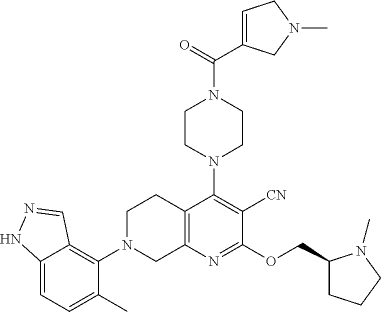 [SMILES: Cc1ccc2[nH]ncc2c1N1CCc2c(nc(OC[C@@H]3CCCN3C)c(C#N)c2N2CCN(C(=O)C3=CCN(C)C3)CC2)C1] | (S)-7-(5-methyl-1H-indazol-4-yl)-4- (4-(1-methyl-2,5-dihydro-1H-pyrrole- 3-carbonyl)piperazin-1-yl)-2-((1- methylpyrrolidin-2-yl)methoxy)- 5,6,7,8-tetrahydro-1,7-naphthyridine- 3-carbonitrile; | 596 |
| 111 | 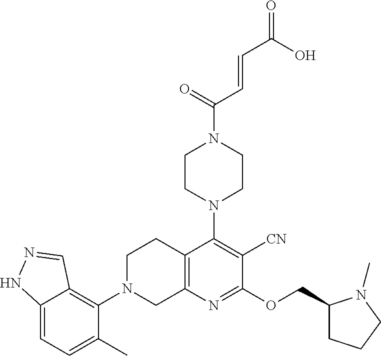 [SMILES: Cc1ccc2[nH]ncc2c1N1CCc2c(nc(OC[C@@H]3CCCN3C)c(C#N)c2N2CCN(C(=O)/C=C/C(=O)O)CC2)C1] | (S,E)-4-(4-(3-cyano-7-(5-methyl-1H- indazol-4-yl)-2-((1-methylpyrrolidin- 2-yl)methoxy)-5,6,7,8-tetrahydro-1,7- naphthyridin-4-yl)piperazin-1-yl)-4- oxobut-2-enoic acid; | 585 |
| 112 | 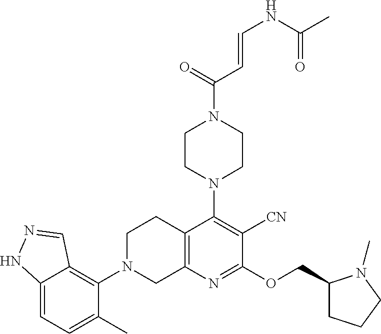 [SMILES: CC(=O)N/C=C/C(=O)N1CCN(c2c(C#N)c(OC[C@@H]3CCCN3C)nc3c2CCN(c2c(C)ccc4[nH]ncc24)C3)CC1] | (S,E)-N-(3-(4-(3-cyano-7-(5-methyl- 1H-indazol-4-yl)-2-((1- methylpyrrolidin-2-yl)methoxy)- 5,6,7,8-tetrahydro-1,7-naphthyridin-4- yl)piperazin-1-yl)-3-oxoprop-1-en-1- yl)acetamide; | 598 |
| 113 | 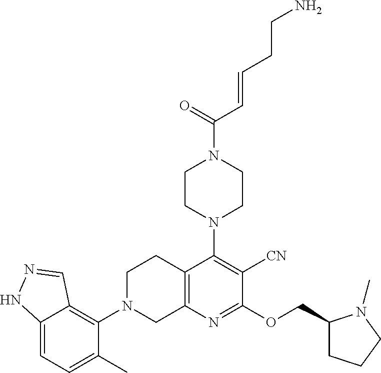 [SMILES: Cc1ccc2[nH]ncc2c1N1CCc2c(nc(OC[C@@H]3CCCN3C)c(C#N)c2N2CCN(C(=O)/C=C/CCN)CC2)C1] | (S,E)-4-(4-(5-aminopent-2- enoyl)piperazin-1-yl)-7-(5-methyl- 1H-indazol-4-yl)-2-((1- methylpyrrolidin-2-yl)methoxy)- 5,6,7,8-tetrahydro-1,7-naphthyridine- 3-carbonitrile; | 584 |
| 114 | 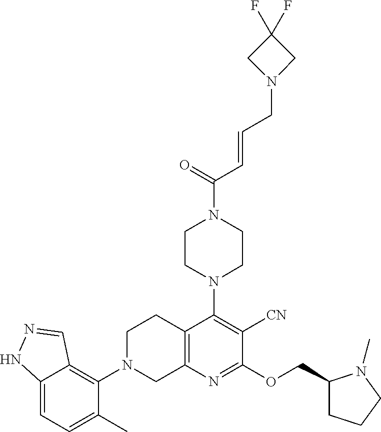 [SMILES: Cc1ccc2[nH]ncc2c1N1CCc2c(nc(OC[C@@H]3CCCN3C)c(C#N)c2N2CCN(C(=O)/C=C/CN3CC(F)(F)C3)CC2)C1] | (S,E)-4-(4-(4-(3,3-difluoroazetidin-1- yl)but-2-enoyl)piperazin-1-yl)-7-(5- methyl-1H-indazol-4-yl)-2-((1- methylpyrrolidin-2-yl)methoxy)- 5,6,7,8-tetrahydro-1,7-naphthyridine- 3-carbonitrile; | 646 |
| 115 | 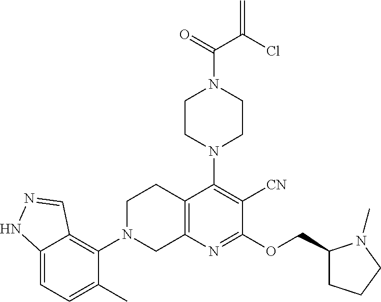 [SMILES: C=C(Cl)C(=O)N1CCN(c2c(C#N)c(OC[C@@H]3CCCN3C)nc3c2CCN(c2c(C)ccc4[nH]ncc24)C3)CC1] | (S)-4-(4-(2-chloroacryloyl)piperazin- 1-yl)-7-(5-methyl-1H-indazol-4-yl)-2- ((1-methylpyrrolidin-2-yl)methoxy)- 5,6,7,8-tetrahydro-1,7-naphthyridine- 3-carbonitrile; | 575 |
| 116 | 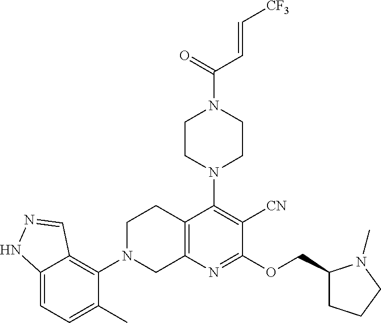 [SMILES: Cc1ccc2[nH]ncc2c1N1CCc2c(nc(OC[C@@H]3CCCN3C)c(C#N)c2N2CCN(C(=O)/C=C/C(F)(F)F)CC2)C1] | (S,E)-7-(5-methyl-1H-indazol-4-yl)-2- ((1-methylpyrrolidin-2-yl)methoxy)- 4-(4-(4,4,4-trifluorobut-2- enoyl)piperazin-1-yl)-5,6,7,8- tetrahydro-1,7-naphthyridine-3- carbonitrile; | 609 |
| 117 | 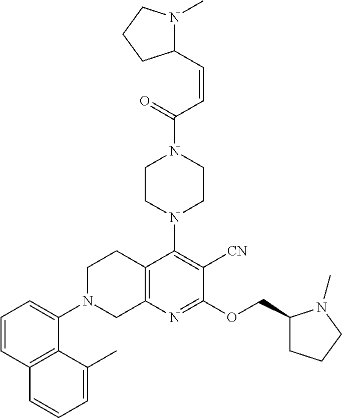 [SMILES: Cc1cccc2cccc(N3CCc4c(nc(OC[C@@H]5CCCN5C)c(C#N)c4N4CCN(C(=O)/C=C\C5CCCN5C)CC4)C3)c12] | 7-(8-methylnaphthalen-1-yl)-4-(4- ((Z)-3-(1-methylpyrrolidin-2- yl)acryloyl)piperazin-1-yl)-2-(((S)-1- methylpyrrolidin-2-yl)methoxy)- 5,6,7,8-tetrahydro-1,7-naphthyridine- 3-carbonitrile; | 634 |
| 118 | 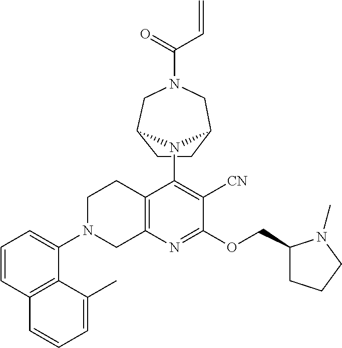 [SMILES: C=CC(=O)N1C[C@H]2CC[C@@H](C1)N2c1c(C#N)c(OC[C@@H]2CCCN2C)nc2c1CCN(c1cccc3cccc(C)c13)C2] | 4-((1R,5S)-3-acryloyl-3,8- diazabicyclo[3.2.1]octan-8-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 577 |
| 119 | 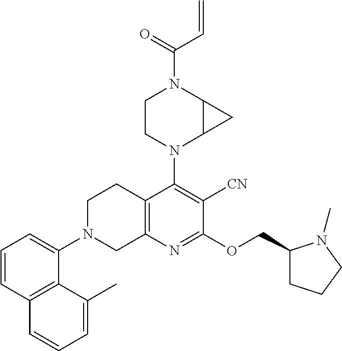 [SMILES: C=CC(=O)N1CCN(c2c(C#N)c(OC[C@@H]3CCCN3C)nc3c2CCN(c2cccc4cccc(C)c24)C3)C2CC21] | 4-(5-acryloyl-2,5- diazabicyclo[4.1.0]heptan-2-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 563 |
| 120 | 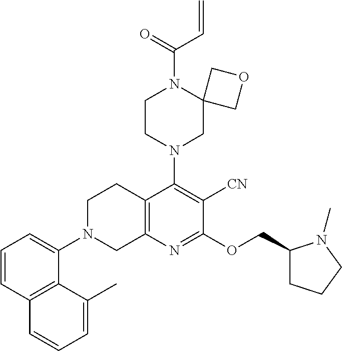 [SMILES: C=CC(=O)N1CCN(c2c(C#N)c(OC[C@@H]3CCCN3C)nc3c2CCN(c2cccc4cccc(C)c24)C3)CC12COC2] | (S)-4-(5-acryloyl-2-oxa-5,8- diazaspiro[3.5]nonan-8-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 593 |
| 121 | 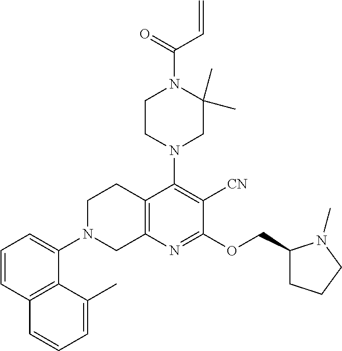 [SMILES: C=CC(=O)N1CCN(c2c(C#N)c(OC[C@@H]3CCCN3C)nc3c2CCN(c2cccc4cccc(C)c24)C3)CC1(C)C] | (S)-4-(4-acryloyl-3,3- dimethylpiperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 579 |
| 122 | 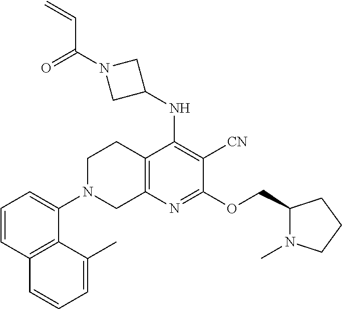 [SMILES: C=CC(=O)N1CC(Nc2c(C#N)c(OC[C@H]3CCCN3C)nc3c2CCN(c2cccc4cccc(C)c24)C3)C1] | (R)-4-((1-acryloylazetidin-3- yl)amino)-7-(8-methylnaphthalen-1- yl)-2-((1-methylpyrrolidin-2- yl)methoxy)-5,6,7,8-tetrahydro-1,7- naphthyridine-3-carbonitrile; | 537 |
| 123 | 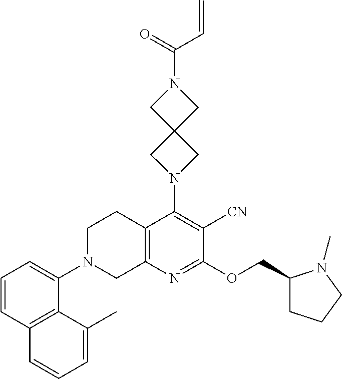 [SMILES: C=CC(=O)N1CC2(C1)CN(c1c(C#N)c(OC[C@@H]3CCCN3C)nc3c1CCN(c1cccc4cccc(C)c14)C3)C2] | (S)-4-(6-acryloyl-2,6- diazaspiro[3.3]heptan-2-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 563 |
| 124 | 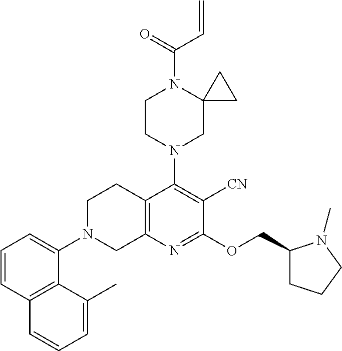 [SMILES: C=CC(=O)N1CCN(c2c(C#N)c(OC[C@@H]3CCCN3C)nc3c2CCN(c2cccc4cccc(C)c24)C3)CC12CC2] | (S)-4-(4-acryloyl-4,7- diazaspiro[2.5]octan-7-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 577 |
| 125 | 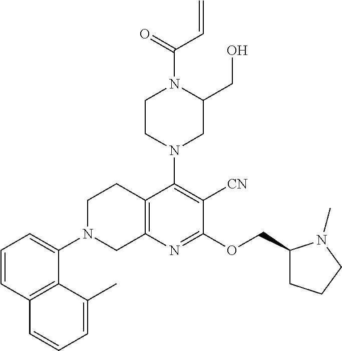 [SMILES: C=CC(=O)N1CCN(c2c(C#N)c(OC[C@@H]3CCCN3C)nc3c2CCN(c2cccc4cccc(C)c24)C3)CC1CO] | 4-(4-acryloyl-3- (hydroxymethyl)piperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 581 |
| 126 | 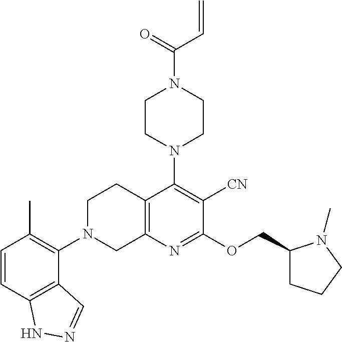 [SMILES: C=CC(=O)N1CCN(c2c(C#N)c(OC[C@@H]3CCCN3C)nc3c2CCN(c2c(C)ccc4[nH]ncc24)C3)CC1] | (S)-4-(4-acryloylpiperazin-1-yl)-7-(5- methyl-1H-indazol-4-yl)-2-((1- methylpyrrolidin-2-yl)methoxy)- 5,6,7,8-tetrahydro-1,7-naphthyridine- 3-carbonitrile; | 541 |
| 127 | 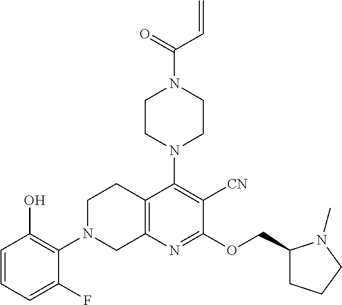 [SMILES: C=CC(=O)N1CCN(c2c(C#N)c(OC[C@@H]3CCCN3C)nc3c2CCN(c2c(O)cccc2F)C3)CC1] | (S)-4-(4-acryloylpiperazin-1-yl)-7-(2- fluoro-6-hydroxyphenyl)-2-((1- methylpyrrolidin-2-yl)methoxy)- 5,6,7,8-tetrahydro-1,7-naphthyridine- 3-carbonitrile; | 521 |
| 128 | 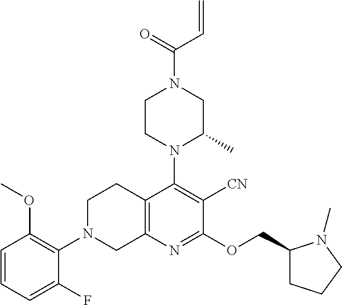 [SMILES: C=CC(=O)N1CCN(c2c(C#N)c(OC[C@@H]3CCCN3C)nc3c2CCN(c2c(F)cccc2OC)C3)[C@@H](C)C1] | 4-((S)-4-acryloyl-2-methylpiperazin- 1-yl)-7-(2-fluoro-6-methoxyphenyl)- 2-(((S)-1-methylpyrrolidin-2- yl)methoxy)-5,6,7,8-tetrahydro-1,7- naphthyridine-3-carbonitrile; | 549 |
| 129 | 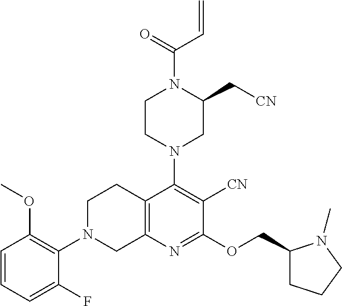 [SMILES: C=CC(=O)N1CCN(c2c(C#N)c(OC[C@@H]3CCCN3C)nc3c2CCN(c2c(F)cccc2OC)C3)C[C@@H]1CC#N] | 4-((S)-4-acryloyl-3- (cyanomethyl)piperazin-1-yl)-7-(2- fluoro-6-methoxyphenyl)-2-(((S)-1- methylpyrrolidin-2-yl)methoxy)- 5,6,7,8-tetrahydro-1,7-naphthyridine- 3-carbonitrile; | 574 |
| 130 | 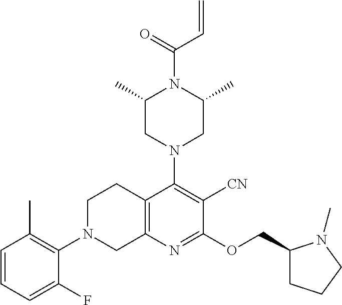 [SMILES: C=CC(=O)N1[C@H](C)CN(c2c(C#N)c(OC[C@@H]3CCCN3C)nc3c2CCN(c2c(C)cccc2F)C3)C[C@@H]1C] | 4-((3R,5S)-4-acryloyl-3,5- dimethylpiperazin-1-yl)-7-(2-fluoro-6- methylphenyl)-2-(((S)-1- methylpyrrolidin-2-yl)methoxy)- 5,6,7,8-tetrahydro-1,7-naphthyridine- 3-carbonitrile; | 547 |
| 131 | 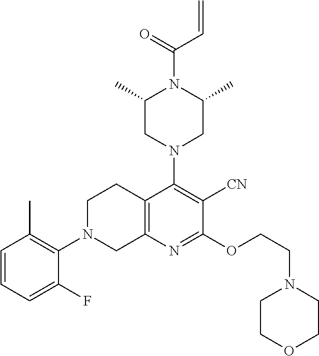 [SMILES: C=CC(=O)N1[C@H](C)CN(c2c(C#N)c(OCCN3CCOCC3)nc3c2CCN(c2c(C)cccc2F)C3)C[C@@H]1C] | 4-((3R,5S)-4-acryloyl-3,5- dimethylpiperazin-1-yl)-7-(2-fluoro-6- methylphenyl)-2-(2- morpholinoethoxy)-5,6,7,8- tetrahydro-1,7-naphthyridine-3- carbonitrile; | 563 |
| 132 | 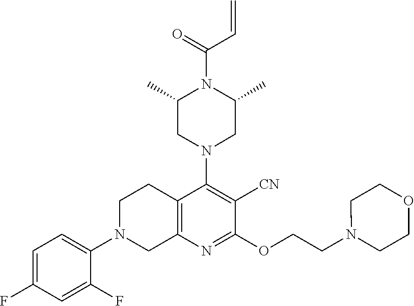 [SMILES: C=CC(=O)N1[C@H](C)CN(c2c(C#N)c(OCCN3CCOCC3)nc3c2CCN(c2ccc(F)cc2F)C3)C[C@@H]1C] | 4-((3R,5S)-4-acryloyl-3,5- dimethylpiperazin-1-yl)-7-(2,4- difluorophenyl)-2-(2- morpholinoethoxy)-5,6,7,8- tetrahydro-1,7-naphthyridine-3- carbonitrile; | 567 |
| 133 | 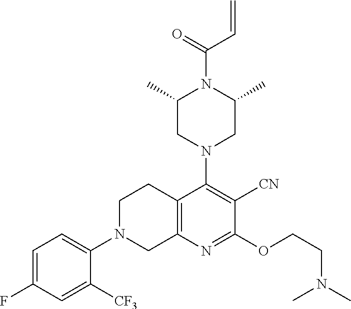 [SMILES: C=CC(=O)N1[C@H](C)CN(c2c(C#N)c(OCCN(C)C)nc3c2CCN(c2ccc(F)cc2C(F)(F)F)C3)C[C@@H]1C] | 4-((3R,5S)-4-acryloyl-3,5- dimethylpiperazin-1-yl)-2-(2- (dimethylamino)ethoxy)-7-(4-fluoro- 2-(trifluoromethyl)phenyl)-5,6,7,8- tetrahydro-1,7-naphthyridine-3- carbonitrile; | 575 |
| 134 | 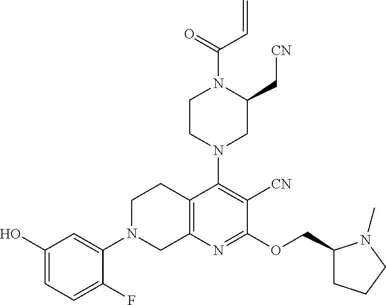 [SMILES: C=CC(=O)N1CCN(c2c(C#N)c(OC[C@@H]3CCCN3C)nc3c2CCN(c2cc(O)ccc2F)C3)C[C@@H]1CC#N] | 4-((S)-4-acryloyl-3- (cyanomethyl)piperazin-1-yl)-7-(2- fluoro-5-hydroxyphenyl)-2-(((S)-1- methylpyrrolidin-2-yl)methoxy)- 5,6,7,8-tetrahydro-1,7-naphthyridine- 3-carbonitrile; | 560 |
| 135 | 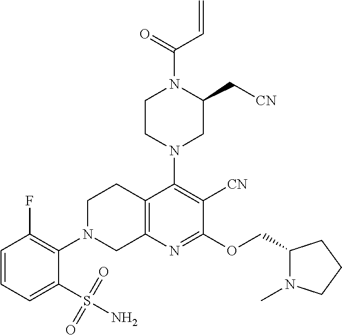 [SMILES: C=CC(=O)N1CCN(c2c(C#N)c(OC[C@@H]3CCCN3C)nc3c2CCN(c2c(F)cccc2S(N)(=O)=O)C3)C[C@@H]1CC#N] | 2-(4-((S)-4-acryloyl-3- (cyanomethyl)piperazin-1-yl)-3- cyano-2-(((S)-1-methylpyrrolidin-2- yl)methoxy)-5,8-dihydro-1,7- naphthyridin-7(6H)-yl)-3- fluorobenzenesulfonamide; | 623 |
| 136 | 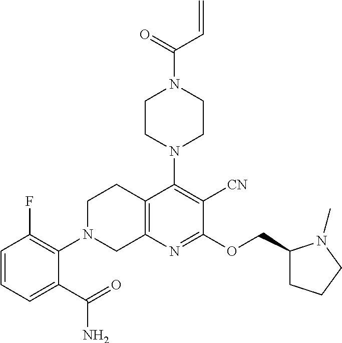 [SMILES: C=CC(=O)N1CCN(c2c(C#N)c(OC[C@@H]3CCCN3C)nc3c2CCN(c2c(F)cccc2C(N)=O)C3)CC1] | (S)-2-(4-(4-acryloylpiperazin-1-yl)-3- cyano-2-((1-methylpyrrolidin-2- yl)methoxy)-5,8-dihydro-1,7- naphthyridin-7(6H)-yl)-3- fluorobenzamide; | 548 |
| 137 | 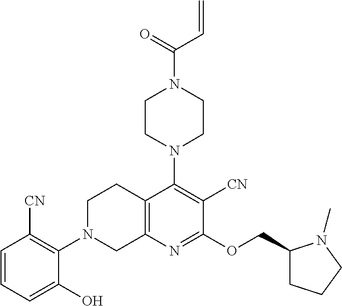 [SMILES: C=CC(=O)N1CCN(c2c(C#N)c(OC[C@@H]3CCCN3C)nc3c2CCN(c2c(O)cccc2C#N)C3)CC1] | (S)-4-(4-acryloylpiperazin-1-yl)-7-(2- cyano-6-hydroxyphenyl)-2-((1- methylpyrrolidin-2-yl)methoxy)- 5,6,7,8-tetrahydro-1,7-naphthyridine- 3-carbonitrile; | 528 |
| 138 | 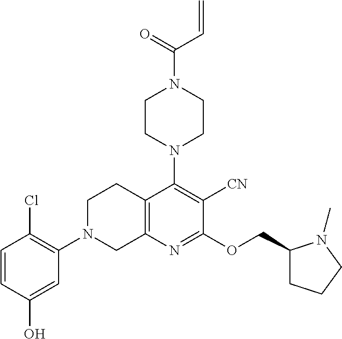 [SMILES: C=CC(=O)N1CCN(c2c(C#N)c(OC[C@@H]3CCCN3C)nc3c2CCN(c2cc(O)ccc2Cl)C3)CC1] | (S)-4-(4-acryloylpiperazin-1-yl)-7-(2- chloro-5-hydroxyphenyl)-2-((1- methylpyrrolidin-2-yl)methoxy)- 5,6,7,8-tetrahydro-1,7-naphthyridine- 3-carbonitrile; | 537 |
| 139 | 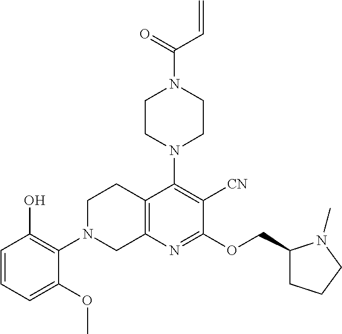 [SMILES: C=CC(=O)N1CCN(c2c(C#N)c(OC[C@@H]3CCCN3C)nc3c2CCN(c2c(O)cccc2OC)C3)CC1] | (S)-4-(4-acryloylpiperazin-1-yl)-7-(2- hydroxy-6-methoxyphenyl)-2-((1- methylpyrrolidin-2-yl)methoxy)- 5,6,7,8-tetrahydro-1,7-naphthyridine- 3-carbonitrile; | 533 |
| 140 | 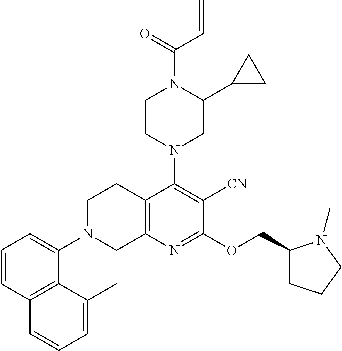 [SMILES: C=CC(=O)N1CCN(c2c(C#N)c(OC[C@@H]3CCCN3C)nc3c2CCN(c2cccc4cccc(C)c24)C3)CC1C1CC1] | 4-(4-acryloyl-3-cyclopropylpiperazin- 1-yl)-7-(8-methylnaphthalen-1-yl)-2- (((S)-1-methylpyrrolidin-2- yl)methoxy)-5,6,7,8-tetrahydro-1,7- naphthyridine-3-carbonitrile; | 591 |
| 141 | 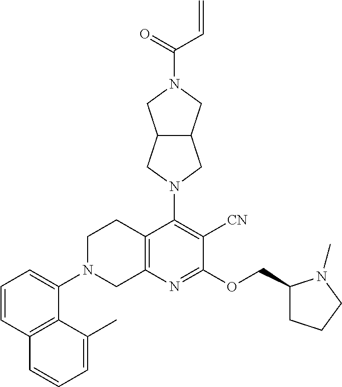 [SMILES: C=CC(=O)N1CC2CN(c3c(C#N)c(OC[C@@H]4CCCN4C)nc4c3CCN(c3cccc5cccc(C)c35)C4)CC2C1] | 4-(5-acryloylhexahydropyrrolo[3,4- c]pyrrol-2(1H)-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 577 |
| 142 | 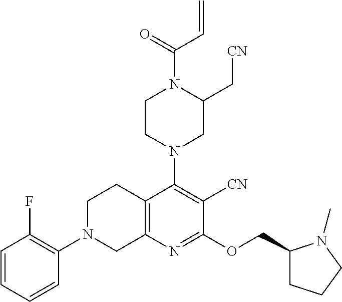 [SMILES: C=CC(=O)N1CCN(c2c(C#N)c(OC[C@@H]3CCCN3C)nc3c2CCN(c2ccccc2F)C3)CC1CC#N] | 4-(4-acryloyl-3- (cyanomethyl)piperazin-1-yl)-7-(2- fluorophenyl)-2-(((S)-1- methylpyrrolidin-2-yl)methoxy)- 5,6,7,8-tetrahydro-1,7-naphthyridine- 3-carbonitrile; | 544 |
| 143 | 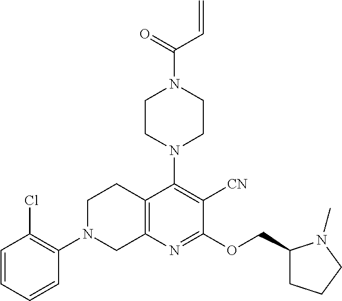 [SMILES: C=CC(=O)N1CCN(c2c(C#N)c(OC[C@@H]3CCCN3C)nc3c2CCN(c2ccccc2Cl)C3)CC1] | (S)-4-(4-acryloylpiperazin-1-yl)-7-(2- chlorophenyl)-2-((1-methylpyrrolidin- 2-yl)methoxy)-5,6,7,8-tetrahydro-1,7- naphthyridine-3-carbonitrile; | 521 |
| 144 | 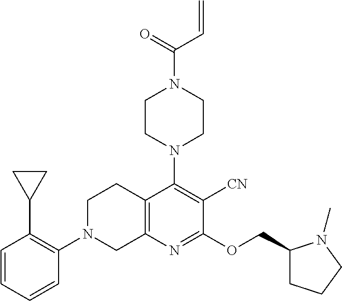 [SMILES: C=CC(=O)N1CCN(c2c(C#N)c(OC[C@@H]3CCCN3C)nc3c2CCN(c2ccccc2C2CC2)C3)CC1] | (S)-4-(4-acryloylpiperazin-1-yl)-7-(2- cyclopropylphenyl)-2-((1- methylpyrrolidin-2-yl)methoxy)- 5,6,7,8-tetrahydro-1,7-naphthyridine- 3-carbonitrile; | 527 |
| 145 | 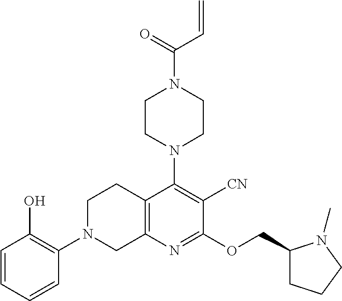 [SMILES: C=CC(=O)N1CCN(c2c(C#N)c(OC[C@@H]3CCCN3C)nc3c2CCN(c2ccccc2O)C3)CC1] | (S)-4-(4-acryloylpiperazin-1-yl)-7-(2- hydroxyphenyl)-2-((1- methylpyrrolidin-2-yl)methoxy)- 5,6,7,8-tetrahydro-1,7-naphthyridine- 3-carbonitrile; | 503 |
| 146 | 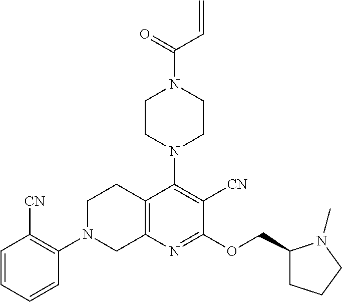 [SMILES: C=CC(=O)N1CCN(c2c(C#N)c(OC[C@@H]3CCCN3C)nc3c2CCN(c2ccccc2C#N)C3)CC1] | (S)-4-(4-acryloylpiperazin-1-yl)-7-(2- cyanophenyl)-2-((1-methylpyrrolidin- 2-yl)methoxy)-5,6,7,8-tetrahydro-1,7- naphthyridine-3-carbonitrile; | 512 |
| 147 | 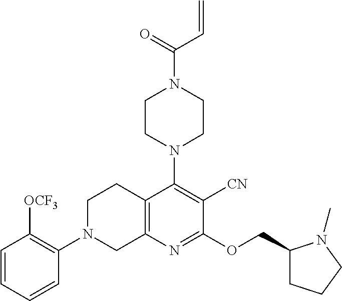 [SMILES: C=CC(=O)N1CCN(c2c(C#N)c(OC[C@@H]3CCCN3C)nc3c2CCN(c2ccccc2OC(F)(F)F)C3)CC1] | (S)-4-(4-acryloylpiperazin-1-yl)-2-((1- methylpyrrolidin-2-yl)methoxy)-7-(2- (trifluoromethoxy)phenyl)-5,6,7,8- tetrahydro-1,7-naphthyridine-3- carbonitrile; | 571 |
| 148 | 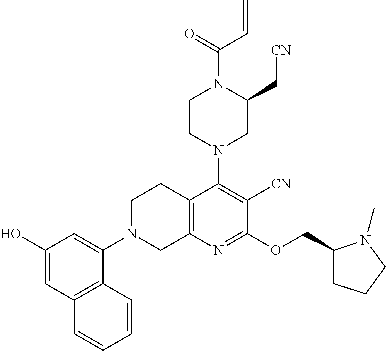 [SMILES: C=CC(=O)N1CCN(c2c(C#N)c(OC[C@@H]3CCCN3C)nc3c2CCN(c2cc(O)cc4ccccc24)C3)C[C@@H]1CC#N] | 4-((S)-4-acryloyl-3- (cyanomethyl)piperazin-1-yl)-7-(3- hydroxynaphthalen-1-yl)-2-(((S)-1- methylpyrrolidin-2-yl)methoxy)- 5,6,7,8-tetrahydro-1,7-naphthyridine- 3-carbonitrile; | 592 |
| 149 | 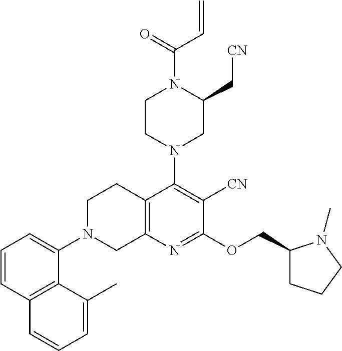 [SMILES: C=CC(=O)N1CCN(c2c(C#N)c(OC[C@@H]3CCCN3C)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 590 |
| 150 | 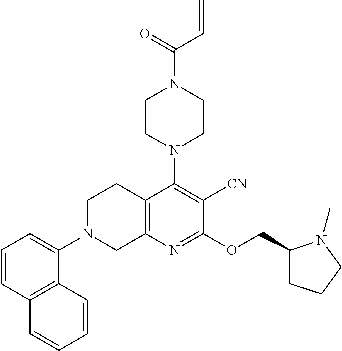 [SMILES: C=CC(=O)N1CCN(c2c(C#N)c(OC[C@@H]3CCCN3C)nc3c2CCN(c2cccc4ccccc24)C3)CC1] | (S)-4-(4-acryloylpiperazin-1-yl)-2-((1- methylpyrrolidin-2-yl)methoxy)-7- (naphthalen-1-yl)-5,6,7,8-tetrahydro- 1,7-naphthyridine-3-carbonitrile; | 537 |
| 151 | 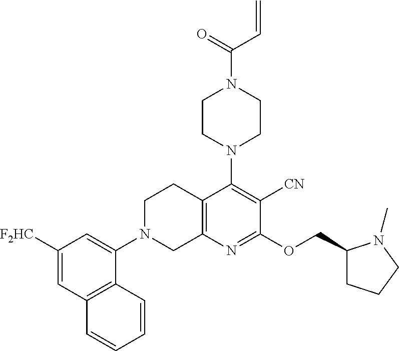 [SMILES: C=CC(=O)N1CCN(c2c(C#N)c(OC[C@@H]3CCCN3C)nc3c2CCN(c2cc(C(F)F)cc4ccccc24)C3)CC1] | (S)-4-(4-acryloylpiperazin-1-yl)-7-(3- (difluoromethyl)naphthalen-1-yl)-2- ((1-methylpyrrolidin-2-yl)methoxy)- 5,6,7,8-tetrahydro-1,7-naphthyridine- 3-carbonitrile; | 587 |
| 152 | 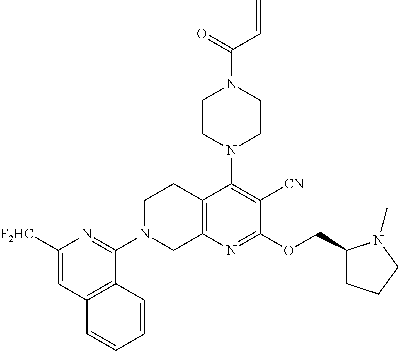 [SMILES: C=CC(=O)N1CCN(c2c(C#N)c(OC[C@@H]3CCCN3C)nc3c2CCN(c2nc(C(F)F)cc4ccccc24)C3)CC1] | (S)-4-(4-acryloylpiperazin-1-yl)-7-(3- (difluoromethyl)isoquinolin-1-yl)-2- ((1-methylpyrrolidin-2-yl)methoxy)- 5,6,7,8-tetrahydro-1,7-naphthyridine- 3-carbonitrile; | 588 |
| 153 | 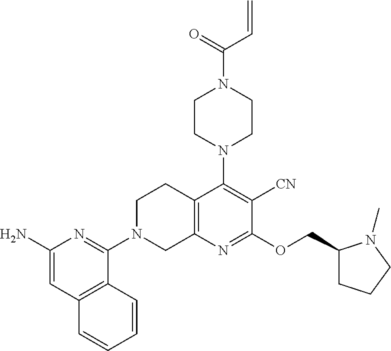 [SMILES: C=CC(=O)N1CCN(c2c(C#N)c(OC[C@@H]3CCCN3C)nc3c2CCN(c2nc(N)cc4ccccc24)C3)CC1] | (S)-4-(4-acryloylpiperazin-1-yl)-7-(3- aminoisoquinolin-1-yl)-2-((1- methylpyrrolidin-2-yl)methoxy)- 5,6,7,8-tetrahydro-1,7-naphthyridine- 3-carbonitrile; | 553 |
| 154 | 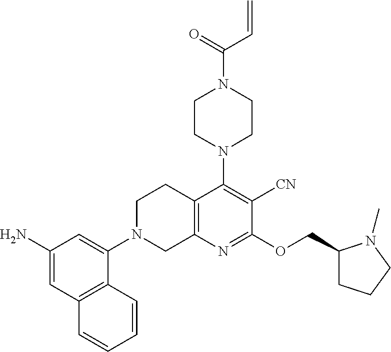 [SMILES: C=CC(=O)N1CCN(c2c(C#N)c(OC[C@@H]3CCCN3C)nc3c2CCN(c2cc(N)cc4ccccc24)C3)CC1] | (S)-4-(4-acryloylpiperazin-1-yl)-7-(3- aminonaphthalen-1-yl)-2-((1- methylpyrrolidin-2-yl)methoxy)- 5,6,7,8-tetrahydro-1,7-naphthyridine- 3-carbonitrile; | 552 |
| 155 | 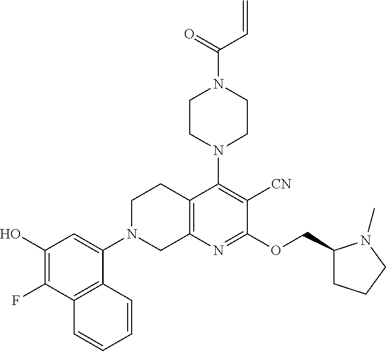 [SMILES: C=CC(=O)N1CCN(c2c(C#N)c(OC[C@@H]3CCCN3C)nc3c2CCN(c2cc(O)c(F)c4ccccc24)C3)CC1] | (S)-4-(4-acryloylpiperazin-1-yl)-7-(4- fluoro-3-hydroxynaphthalen-1-yl)-2- ((1-methylpyrrolidin-2-yl)methoxy)- 5,6,7,8-tetrahydro-1,7-naphthyridine- 3-carbonitrile; | 571 |
| 156 | 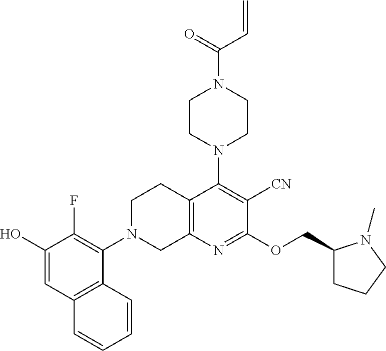 [SMILES: C=CC(=O)N1CCN(c2c(C#N)c(OC[C@@H]3CCCN3C)nc3c2CCN(c2c(F)c(O)cc4ccccc24)C3)CC1] | (S)-4-(4-acryloylpiperazin-1-yl)-7-(2- fluoro-3-hydroxynaphthalen-1-yl)-2- ((1-methylpyrrolidin-2-yl)methoxy)- 5,6,7,8-tetrahydro-1,7-naphthyridine- 3-carbonitrile; | 571 |
| 157 | 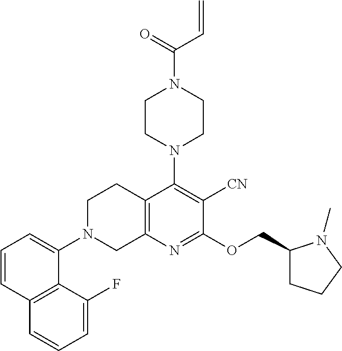 [SMILES: C=CC(=O)N1CCN(c2c(C#N)c(OC[C@@H]3CCCN3C)nc3c2CCN(c2cccc4cccc(F)c24)C3)CC1] | (S)-4-(4-acryloylpiperazin-1-yl)-7-(8- fluoronaphthalen-1-yl)-2-((1- methylpyrrolidin-2-yl)methoxy)- 5,6,7,8-tetrahydro-1,7-naphthyridine- 3-carbonitrile; | 555 |
| 158 | 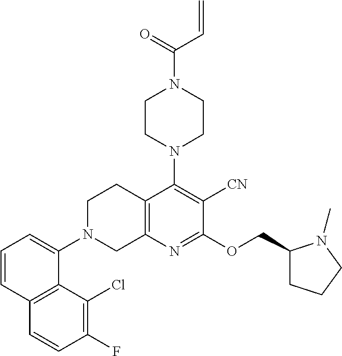 [SMILES: C=CC(=O)N1CCN(c2c(C#N)c(OC[C@@H]3CCCN3C)nc3c2CCN(c2cccc4ccc(F)c(Cl)c24)C3)CC1] | (S)-4-(4-acryloylpiperazin-1-yl)-7-(8- chloro-7-fluoronaphthalen-1-yl)-2-((1- methylpyrrolidin-2-yl)methoxy)- 5,6,7,8-tetrahydro-1,7-naphthyridine- 3-carbonitrile; | 589 |
| 159 | 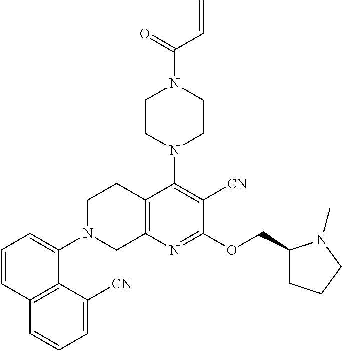 [SMILES: C=CC(=O)N1CCN(c2c(C#N)c(OC[C@@H]3CCCN3C)nc3c2CCN(c2cccc4cccc(C#N)c24)C3)CC1] | (S)-4-(4-acryloylpiperazin-1-yl)-7-(8- cyanonaphthalen-1-yl)-2-((1- methylpyrrolidin-2-yl)methoxy)- 5,6,7,8-tetrahydro-1,7-naphthyridine- 3-carbonitrile; | 562 |
| 160 | 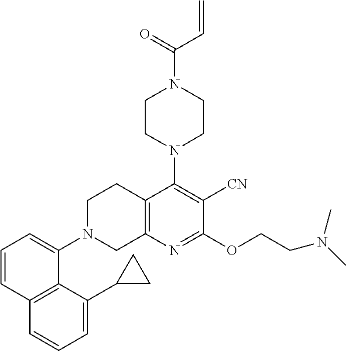 [SMILES: C=CC(=O)N1CCN(c2c(C#N)c(OCCN(C)C)nc3c2CCN(c2cccc4cccc(C5CC5)c24)C3)CC1] | 4-(4-acryloylpiperazin-1-yl)-7-(8- cyclopropylnaphthalen-1-yl)-2-(2- (dimethylamino)ethoxy)-5,6,7,8- tetrahydro-1,7-naphthyridine-3- carbonitrile; | 551 |
| 161 | 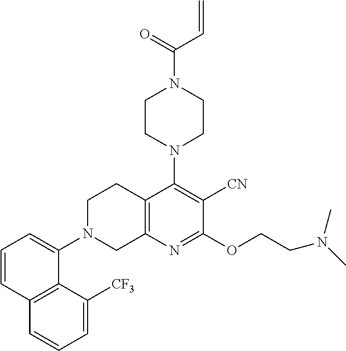 [SMILES: C=CC(=O)N1CCN(c2c(C#N)c(OCCN(C)C)nc3c2CCN(c2cccc4cccc(C(F)(F)F)c24)C3)CC1] | 4-(4-acryloylpiperazin-1-yl)-2-(2- (dimethylamino)ethoxy)-7-(8- (trifluoromethyl)naphthalen-1-yl)- 5,6,7,8-tetrahydro-1,7-naphthyridine- 3-carbonitrile; | 579 |
| 162 | 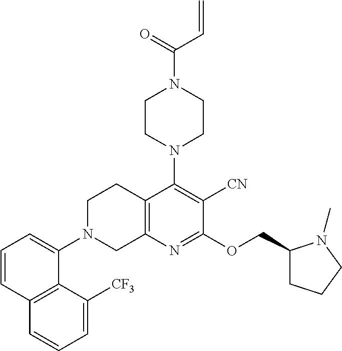 [SMILES: C=CC(=O)N1CCN(c2c(C#N)c(OC[C@@H]3CCCN3C)nc3c2CCN(c2cccc4cccc(C(F)(F)F)c24)C3)CC1] | (S)-4-(4-acryloylpiperazin-1-yl)-2-((1- methylpyrrolidin-2-yl)methoxy)-7-(8- (trifluoromethyl)naphthalen-1-yl)- 5,6,7,8-tetrahydro-1,7-naphthyridine- 3-carbonitrile; | 605 |
| 163 | 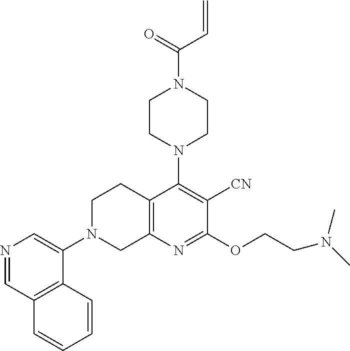 [SMILES: C=CC(=O)N1CCN(c2c(C#N)c(OCCN(C)C)nc3c2CCN(c2cncc4ccccc24)C3)CC1] | 4-(4-acryloylpiperazin-1-yl)-2-(2- (dimethylamino)ethoxy)-7- (isoquinolin-4-yl)-5,6,7,8-tetrahydro- 1,7-naphthyridine-3-carbonitrile; | 512 |
| 164 | 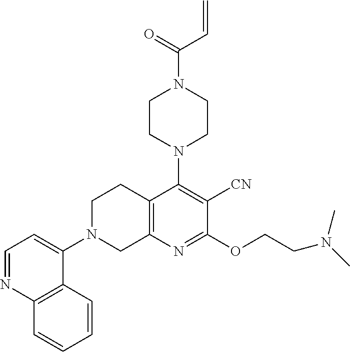 [SMILES: C=CC(=O)N1CCN(c2c(C#N)c(OCCN(C)C)nc3c2CCN(c2ccnc4ccccc24)C3)CC1] | 4-(4-acryloylpiperazin-1-yl)-2-(2- (dimethylamino)ethoxy)-7-(quinolin- 4-yl)-5,6,7,8-tetrahydro-1,7- naphthyridine-3-carbonitrile; | 512 |
| 165 | 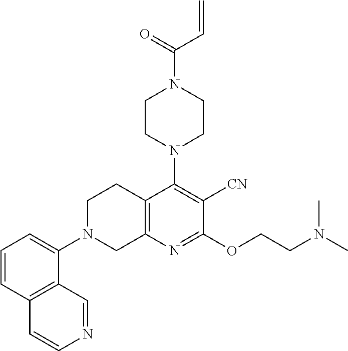 [SMILES: C=CC(=O)N1CCN(c2c(C#N)c(OCCN(C)C)nc3c2CCN(c2cccc4ccncc24)C3)CC1] | 4-(4-acryloylpiperazin-1-yl)-2-(2- (dimethylamino)ethoxy)-7- (isoquinolin-8-yl)-5,6,7,8-tetrahydro- 1,7-naphthyridine-3-carbonitrile; | 512 |
| 166 | 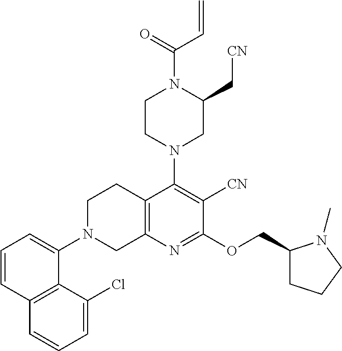 [SMILES: C=CC(=O)N1CCN(c2c(C#N)c(OC[C@@H]3CCCN3C)nc3c2CCN(c2cccc4cccc(Cl)c24)C3)C[C@@H]1CC#N] | 4-((S)-4-acryloyl-3- (cyanomethyl)piperazin-1-yl)-7-(8- chloronaphthalen-1-yl)-2-(((S)-1- methylpyrrolidin-2-yl)methoxy)- 5,6,7,8-tetrahydro-1,7-naphthyridine- 3-carbonitrile; | 610 |
| 167 | 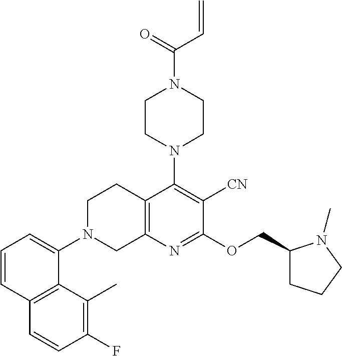 [SMILES: C=CC(=O)N1CCN(c2c(C#N)c(OC[C@@H]3CCCN3C)nc3c2CCN(c2cccc4ccc(F)c(C)c24)C3)CC1] | (S)-4-(4-acryloylpiperazin-1-yl)-7-(7- fluoro-8-methylnaphthalen-1-yl)-2- ((1-methylpyrrolidin-2-yl)methoxy)- 5,6,7,8-tetrahydro-1,7-naphthyridine- 3-carbonitrile; | 569 |
| 168 | 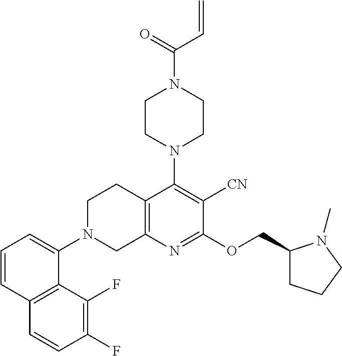 [SMILES: C=CC(=O)N1CCN(c2c(C#N)c(OC[C@@H]3CCCN3C)nc3c2CCN(c2cccc4ccc(F)c(F)c24)C3)CC1] | (S)-4-(4-acryloylpiperazin-1-yl)-7- (7,8-difluoronaphthalen-1-yl)-2-((1- methylpyrrolidin-2-yl)methoxy)- 5,6,7,8-tetrahydro-1,7-naphthyridine- 3-carbonitrile; | 573 |
| 169 | 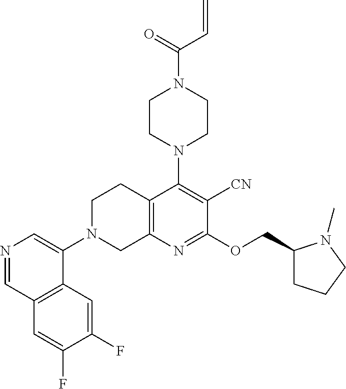 [SMILES: C=CC(=O)N1CCN(c2c(C#N)c(OC[C@@H]3CCCN3C)nc3c2CCN(c2cncc4cc(F)c(F)cc24)C3)CC1] | (S)-4-(4-acryloylpiperazin-1-yl)-7- (6,7-difluoroisoquinolin-4-yl)-2-((1- methylpyrrolidin-2-yl)methoxy)- 5,6,7,8-tetrahydro-1,7-naphthyridine- 3-carbonitrile; | 574 |
| 170 | 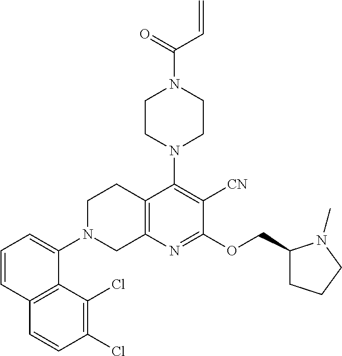 [SMILES: C=CC(=O)N1CCN(c2c(C#N)c(OC[C@@H]3CCCN3C)nc3c2CCN(c2cccc4ccc(Cl)c(Cl)c24)C3)CC1] | (S)-4-(4-acryloylpiperazin-1-yl)-7- (7,8-dichloronaphthalen-1-yl)-2-((1- methylpyrrolidin-2-yl)methoxy)- 5,6,7,8-tetrahydro-1,7-naphthyridine- 3-carbonitrile; | 605 |
| 171 | 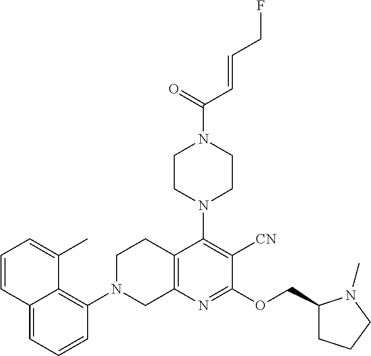 [SMILES: Cc1cccc2cccc(N3CCc4c(nc(OC[C@@H]5CCCN5C)c(C#N)c4N4CCN(C(=O)/C=C/CF)CC4)C3)c12] | (S,E)-4-(4-(4-fluorobut-2- enoyl)piperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 583 |
| 172 | 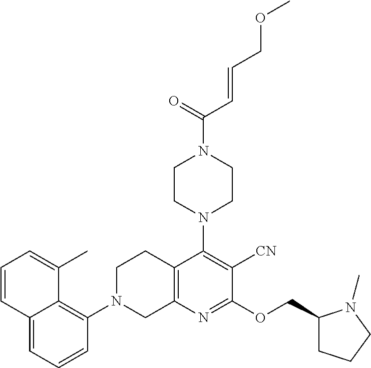 [SMILES: COC/C=C/C(=O)N1CCN(c2c(C#N)c(OC[C@@H]3CCCN3C)nc3c2CCN(c2cccc4cccc(C)c24)C3)CC1] | (S,E)-4-(4-(4-methoxybut-2- enoyl)piperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 595 |
| 173 | 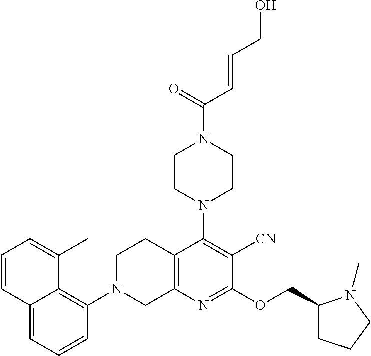 [SMILES: Cc1cccc2cccc(N3CCc4c(nc(OC[C@@H]5CCCN5C)c(C#N)c4N4CCN(C(=O)/C=C/CO)CC4)C3)c12] | (S,E)-4-(4-(4-hydroxybut-2- enoyl)piperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 581 |
| 174 | 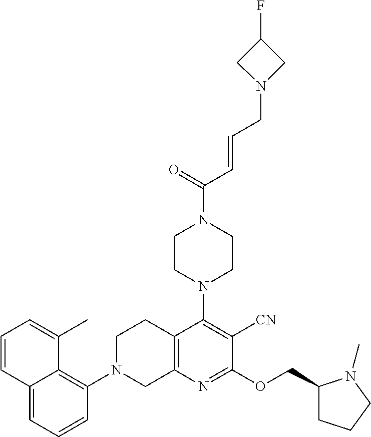 [SMILES: Cc1cccc2cccc(N3CCc4c(nc(OC[C@@H]5CCCN5C)c(C#N)c4N4CCN(C(=O)/C=C/CN5CC(F)C5)CC4)C3)c12] | (S,E)-4-(4-(4-(3-fluoroazetidin-1- yl)but-2-enoyl)piperazin-1-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 638 |
| 175 | 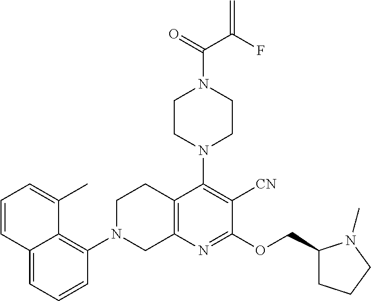 [SMILES: C=C(F)C(=O)N1CCN(c2c(C#N)c(OC[C@@H]3CCCN3C)nc3c2CCN(c2cccc4cccc(C)c24)C3)CC1] | (S)-4-(4-(2-fluoroacryloyl)piperazin- 1-yl)-7-(8-methylnaphthalen-1-yl)-2- ((1-methylpyrrolidin-2-yl)methoxy)- 5,6,7,8-tetrahydro-1,7-naphthyridine- 3-carbonitrile; | 569 |
| 176 | 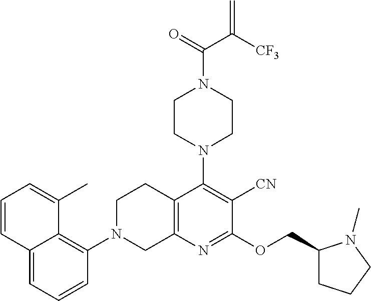 [SMILES: C=C(C(=O)N1CCN(c2c(C#N)c(OC[C@@H]3CCCN3C)nc3c2CCN(c2cccc4cccc(C)c24)C3)CC1)C(F)(F)F] | (S)-7-(8-methylnaphthalen-1-yl)-2- ((1-methylpyrrolidin-2-yl)methoxy)- 4-(4-(2- (trifluoromethyl)acryloyl)piperazin-1- yl)-5,6,7,8-tetrahydro-1,7- naphthyridine-3-carbonitrile; | 619 |
| 177 | 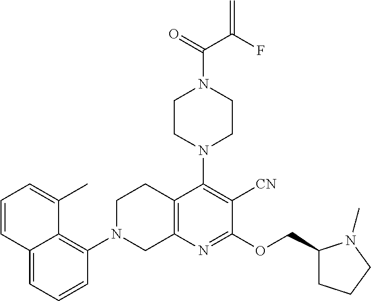 [SMILES: C=C(F)C(=O)N1CCN(c2c(C#N)c(OC[C@@H]3CCCN3C)nc3c2CCN(c2cccc4cccc(C)c24)C3)CC1] | (S)-4-(4-(2-fluoroacryloyl)piperazin- 1-yl)-7-(8-methylnaphthalen-1-yl)-2- ((1-methylpyrrolidin-2-yl)methoxy)- 5,6,7,8-tetrahydro-1,7-naphthyridine- 3-carbonitrile; | 569 |
| 178 | 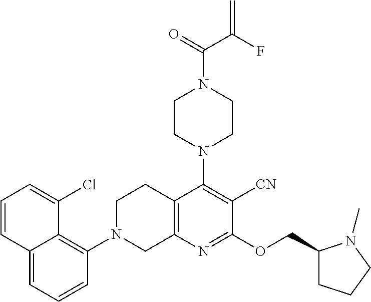 [SMILES: C=C(F)C(=O)N1CCN(c2c(C#N)c(OC[C@@H]3CCCN3C)nc3c2CCN(c2cccc4cccc(Cl)c24)C3)CC1] | (S)-7-(8-chloronaphthalen-1-yl)-4-(4- (2-fluoroacryloyl)piperazin-1-yl)-2- ((1-methylpyrrolidin-2-yl)methoxy)- 5,6,7,8-tetrahydro-1,7-naphthyridine- 3-carbonitrile; | 589 |
| 179 | 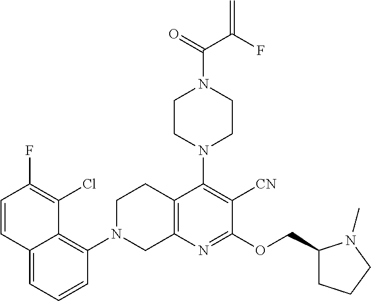 [SMILES: C=C(F)C(=O)N1CCN(c2c(C#N)c(OC[C@@H]3CCCN3C)nc3c2CCN(c2cccc4ccc(F)c(Cl)c24)C3)CC1] | (S)-7-(8-chloro-7-fluoronaphthalen-1- yl)-4-(4-(2-fluoroacryloyl)piperazin- 1-yl)-2-((1-methylpyrrolidin-2- yl)methoxy)-5,6,7,8-tetrahydro-1,7- naphthyridine-3-carbonitrile; | 607 |
| 180 | 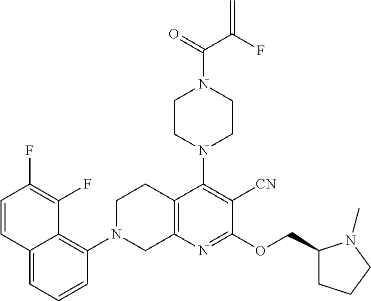 [SMILES: C=C(F)C(=O)N1CCN(c2c(C#N)c(OC[C@@H]3CCCN3C)nc3c2CCN(c2cccc4ccc(F)c(F)c24)C3)CC1] | (S)-7-(7,8-difluoronaphthalen-1-yl)-4- (4-(2-fluoroacryloyl)piperazin-1-yl)- 2-((1-methylpyrrolidin-2- yl)methoxy)-5,6,7,8-tetrahydro-1,7- naphthyridine-3-carbonitrile; | 591 |
| 181 | 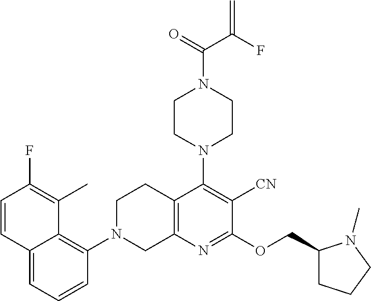 [SMILES: C=C(F)C(=O)N1CCN(c2c(C#N)c(OC[C@@H]3CCCN3C)nc3c2CCN(c2cccc4ccc(F)c(C)c24)C3)CC1] | (S)-7-(7-fluoro-8-methylnaphthalen-1- yl)-4-(4-(2-fluoroacryloyl)piperazin- 1-yl)-2-((1-methylpyrrolidin-2- yl)methoxy)-5,6,7,8-tetrahydro-1,7- naphthyridine-3-carbonitrile; | 587 |
| 182 | 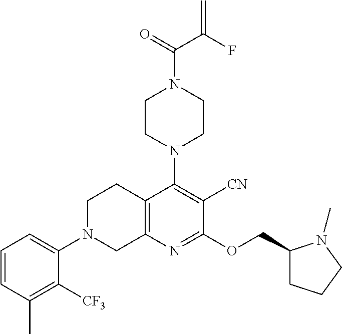 [SMILES: C=C(F)C(=O)N1CCN(c2c(C#N)c(OC[C@@H]3CCCN3C)nc3c2CCN(c2cccc(C)c2C(F)(F)F)C3)CC1] | (S)-4-(4-(2-fluoroacryloyl)piperazin- 1-yl)-7-(3-methyl-2- (trifluoromethyl)phenyl)-2-((1- methylpyrrolidin-2-yl)methoxy)- 5,6,7,8-tetrahydro-1,7-naphthyridine- 3-carbonitrile; | 587 |
| 183 | 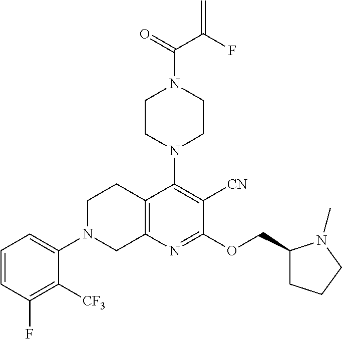 [SMILES: C=C(F)C(=O)N1CCN(c2c(C#N)c(OC[C@@H]3CCCN3C)nc3c2CCN(c2cccc(F)c2C(F)(F)F)C3)CC1] | (S)-7-(3-fluoro-2- (trifluoromethyl)phenyl)-4-(4-(2- fluoroacryloyl)piperazin-1-yl)-2-((1- methylpyrrolidin-2-yl)methoxy)- 5,6,7,8-tetrahydro-1,7-naphthyridine- 3-carbonitrile; | 591 |
| 184 | 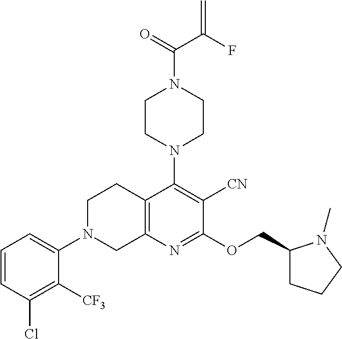 [SMILES: C=C(F)C(=O)N1CCN(c2c(C#N)c(OC[C@@H]3CCCN3C)nc3c2CCN(c2cccc(Cl)c2C(F)(F)F)C3)CC1] | (S)-7-(3-chloro-2- (trifluoromethyl)phenyl)-4-(4-(2- fluoroacryloyl)piperazin-1-yl)-2-((1- methylpyrrolidin-2-yl)methoxy)- 5,6,7,8-tetrahydro-1,7-naphthyridine- 3-carbonitrile; | 607 |
| 185 | 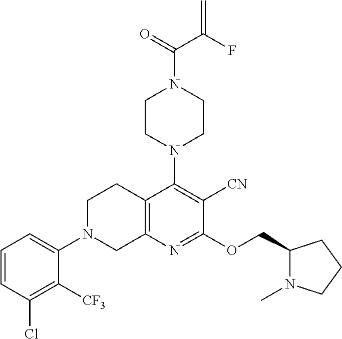 [SMILES: C=C(F)C(=O)N1CCN(c2c(C#N)c(OC[C@H]3CCCN3C)nc3c2CCN(c2cccc(Cl)c2C(F)(F)F)C3)CC1] | (R)-7-(3-chloro-2- (trifluoromethyl)phenyl)-4-(4-(2- fluoroacryloyl)piperazin-1-yl)-2-((1- methylpyrrolidin-2-yl)methoxy)- 5,6,7,8-tetrahydro-1,7-naphthyridine- 3-carbonitrile; | 607 |
| 186 | 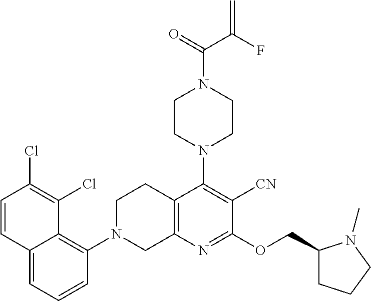 [SMILES: C=C(F)C(=O)N1CCN(c2c(C#N)c(OC[C@@H]3CCCN3C)nc3c2CCN(c2cccc4ccc(Cl)c(Cl)c24)C3)CC1] | (S)-7-(7,8-dichloronaphthalen-1-yl)-4- (4-(2-fluoroacryloyl)piperazin-1-yl)- 2-((1-methylpyrrolidin-2- yl)methoxy)-5,6,7,8-tetrahydro-1,7- naphthyridine-3-carbonitrile; | 623 |
| 187 | 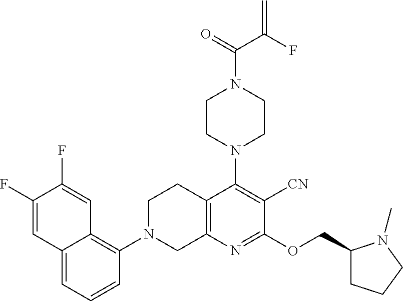 [SMILES: C=C(F)C(=O)N1CCN(c2c(C#N)c(OC[C@@H]3CCCN3C)nc3c2CCN(c2cccc4cc(F)c(F)cc24)C3)CC1] | (S)-7-(6,7-difluoronaphthalen-1-yl)-4- (4-(2-fluoroacryloyl)piperazin-1-yl)- 2-((1-methylpyrrolidin-2- yl)methoxy)-5,6,7,8-tetrahydro-1,7- naphthyridine-3-carbonitrile; | 591 |
| 188 | 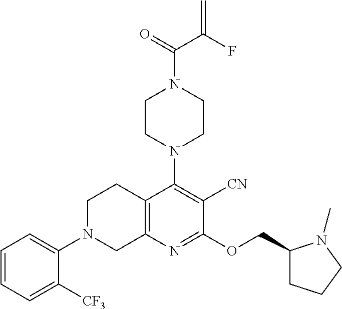 [SMILES: C=C(F)C(=O)N1CCN(c2c(C#N)c(OC[C@@H]3CCCN3C)nc3c2CCN(c2ccccc2C(F)(F)F)C3)CC1] | (S)-4-(4-(2-fluoroacryloyl)piperazin- 1-yl)-2-((1-methylpyrrolidin-2- yl)methoxy)-7-(2- (trifluoromethyl)phenyl)-5,6,7,8- tetrahydro-1,7-naphthyridine-3- carbonitrile; | 573 |
| 189 | 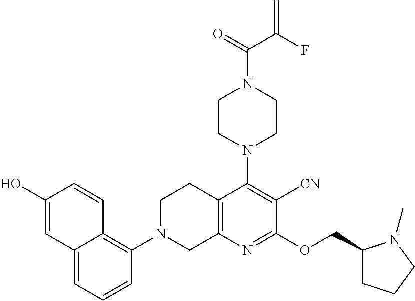 [SMILES: C=C(F)C(=O)N1CCN(c2c(C#N)c(OC[C@@H]3CCCN3C)nc3c2CCN(c2cccc4cc(O)ccc24)C3)CC1] | (S)-4-(4-(2-fluoroacryloyl)piperazin- 1-yl)-7-(6-hydroxynaphthalen-1-yl)-2- ((1-methylpyrrolidin-2-yl)methoxy)- 5,6,7,8-tetrahydro-1,7-naphthyridine- 3-carbonitrile; | 571 |
| 190 | 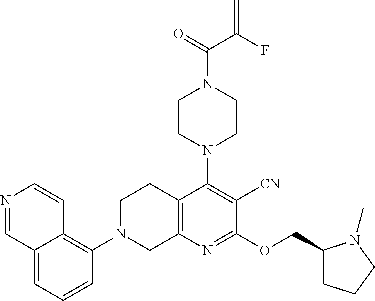 [SMILES: C=C(F)C(=O)N1CCN(c2c(C#N)c(OC[C@@H]3CCCN3C)nc3c2CCN(c2cccc4cnccc24)C3)CC1] | (S)-4-(4-(2-fluoroacryloyl)piperazin- 1-yl)-7-(isoquinolin-5-yl)-2-((1- methylpyrrolidin-2-yl)methoxy)- 5,6,7,8-tetrahydro-1,7-naphthyridine- 3-carbonitrile; | 556 |
| 191 | 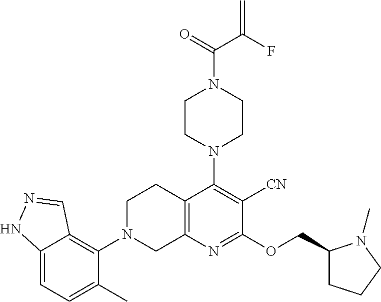 [SMILES: C=C(F)C(=O)N1CCN(c2c(C#N)c(OC[C@@H]3CCCN3C)nc3c2CCN(c2c(C)ccc4[nH]ncc24)C3)CC1] | (S)-4-(4-(2-fluoroacryloyl)piperazin- 1-yl)-7-(5-methyl-1H-indazol-4-yl)-2- ((1-methylpyrrolidin-2-yl)methoxy)- 5,6,7,8-tetrahydro-1,7-naphthyridine- 3-carbonitrile; | 559 |
| 192 | 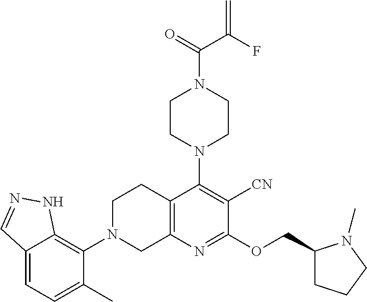 [SMILES: C=C(F)C(=O)N1CCN(c2c(C#N)c(OC[C@@H]3CCCN3C)nc3c2CCN(c2c(C)ccc4cn[nH]c24)C3)CC1] | (S)-4-(4-(2-fluoroacryloyl)piperazin- 1-yl)-7-(6-methyl-1H-indazol-7-yl)-2- ((1-methylpyrrolidin-2-yl)methoxy)- 5,6,7,8-tetrahydro-1,7-naphthyridine- 3-carbonitrile; | 559 |
| 193 | 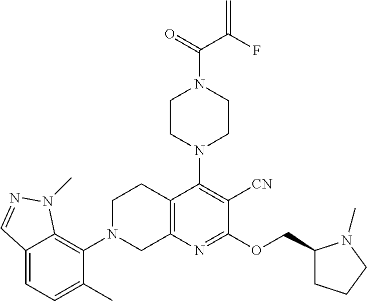 [SMILES: C=C(F)C(=O)N1CCN(c2c(C#N)c(OC[C@@H]3CCCN3C)nc3c2CCN(c2c(C)ccc4cnn(C)c24)C3)CC1] | (S)-7-(1,6-dimethyl-1H-indazol-7-yl)- 4-(4-(2-fluoroacryloyl)piperazin-1- yl)-2-((1-methylpyrrolidin-2- yl)methoxy)-5,6,7,8-tetrahydro-1,7- naphthyridine-3-carbonitrile; | 573 |
| 194 | 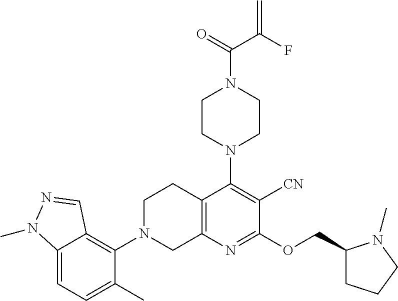 [SMILES: C=C(F)C(=O)N1CCN(c2c(C#N)c(OC[C@@H]3CCCN3C)nc3c2CCN(c2c(C)ccc4c2cnn4C)C3)CC1] | (S)-7-(1,5-dimethyl-1H-indazol-4-yl)- 4-(4-(2-fluoroacryloyl)piperazin-1- yl)-2-((1-methylpyrrolidin-2- yl)methoxy)-5,6,7,8-tetrahydro-1,7- naphthyridine-3-carbonitrile; | 573 |
| 195 | 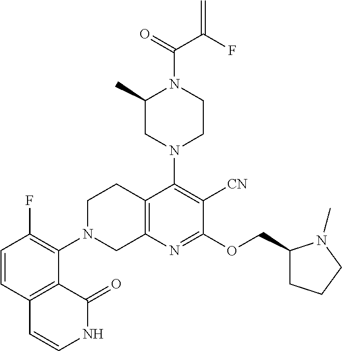 [SMILES: C=C(F)C(=O)N1CCN(c2c(C#N)c(OC[C@@H]3CCCN3C)nc3c2CCN(c2c(F)ccc4cc[nH]c(=O)c24)C3)C[C@H]1C] | 7-(7-fluoro-1-oxo-1,2- dihydroisoquinolin-8-yl)-4-((R)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 604 |
| 196 | 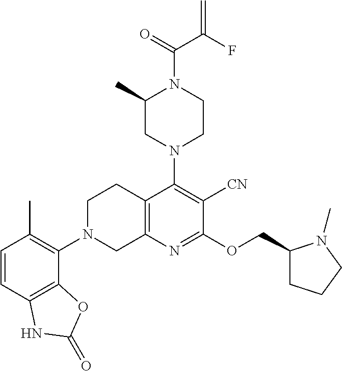 [SMILES: C=C(F)C(=O)N1CCN(c2c(C#N)c(OC[C@@H]3CCCN3C)nc3c2CCN(c2c(C)ccc4[nH]c(=O)oc24)C3)C[C@H]1C] | 4-((R)-4-(2-fluoroacryloyl)-3- methylpiperazin-1-yl)-7-(6-methyl-2- oxo-2,3-dihydrobenzo[d]oxazol-7-yl)- 2-(((S)-1-methylpyrrolidin-2- yl)methoxy)-5,6,7,8-tetrahydro-1,7- naphthyridine-3-carbonitrile; | 590 |
| 197 | 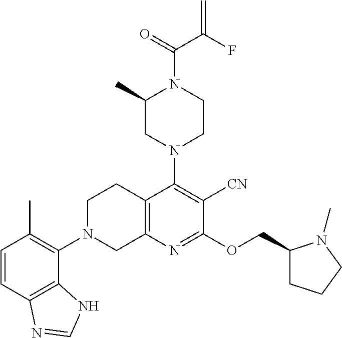 [SMILES: C=C(F)C(=O)N1CCN(c2c(C#N)c(OC[C@@H]3CCCN3C)nc3c2CCN(c2c(C)ccc4nc[nH]c24)C3)C[C@H]1C] | 4-((R)-4-(2-fluoroacryloyl)-3- methylpiperazin-1-yl)-7-(6-methyl- 1H-benzo[d]imidazol-7-yl)-2-(((S)-1- methylpyrrolidin-2-yl)methoxy)- 5,6,7,8-tetrahydro-1,7-naphthyridine- 3-carbonitrile; | 573 |
| 198 | 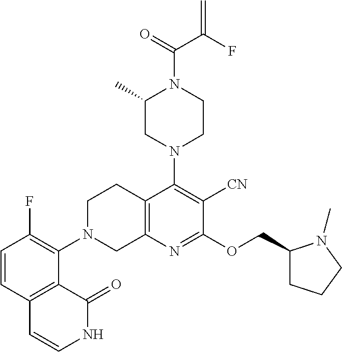 [SMILES: C=C(F)C(=O)N1CCN(c2c(C#N)c(OC[C@@H]3CCCN3C)nc3c2CCN(c2c(F)ccc4cc[nH]c(=O)c24)C3)C[C@@H]1C] | 7-(7-fluoro-1-oxo-1,2- dihydroisoquinolin-8-yl)-4-((S)-4-(2- fluoroacryloyl)-3-methylpiperazin-1- yl)-2-(((S)-1-methylpyrrolidin-2- yl)methoxy)-5,6,7,8-tetrahydro-1,7- naphthyridine-3-carbonitrile; | 604 |
| 199 | 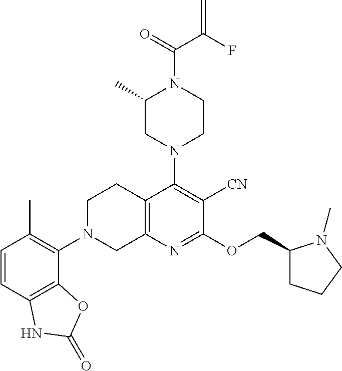 [SMILES: C=C(F)C(=O)N1CCN(c2c(C#N)c(OC[C@@H]3CCCN3C)nc3c2CCN(c2c(C)ccc4[nH]c(=O)oc24)C3)C[C@@H]1C] | 4-((S)-4-(2-fluoroacryloyl)-3- methylpiperazin-1-yl)-7-(6-methyl-2- oxo-2,3-dihydrobenzo[d]oxazol-7-yl)- 2-(((S)-1-methylpyrrolidin-2- yl)methoxy)-5,6,7,8-tetrahydro-1,7- naphthyridine-3-carbonitrile; | 590 |
| 200 | 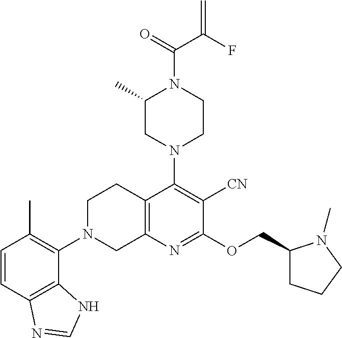 [SMILES: C=C(F)C(=O)N1CCN(c2c(C#N)c(OC[C@@H]3CCCN3C)nc3c2CCN(c2c(C)ccc4nc[nH]c24)C3)C[C@@H]1C] | 4-((S)-4-(2-fluoroacryloyl)-3- methylpiperazin-1-yl)-7-(6-methyl- 1H-benzo[d]imidazol-7-yl)-2-(((S)-1- methylpyrrolidin-2-yl)methoxy)- 5,6,7,8-tetrahydro-1,7-naphthyridine- 3-carbonitrile; | 573 |
| 201 | 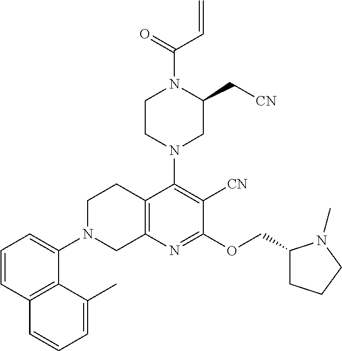 [SMILES: C=CC(=O)N1CCN(c2c(C#N)c(OC[C@H]3CCCN3C)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((R)-1- methylpyrrolidin-2-yl)methoxy)- 5,6,7,8-tetrahydro-1,7-naphthyridine- 3-carbonitrile; | 590 |
| 202 | 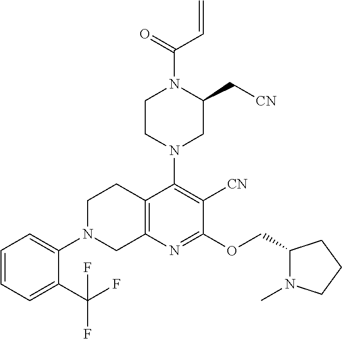 [SMILES: C=CC(=O)N1CCN(c2c(C#N)c(OC[C@@H]3CCCN3C)nc3c2CCN(c2ccccc2C(F)(F)F)C3)C[C@@H]1CC#N] | 4-((S)-4-acryloyl-3- (cyanomethyl)piperazin-1-yl)-2-(((S)- 1-methylpyrrolidin-2-yl)methoxy)-7- (2-(trifluoromethyl)phenyl)-5,6,7,8- tetrahydro-1,7-naphthyridine-3- carbonitrile; | 594 |
| 203 | 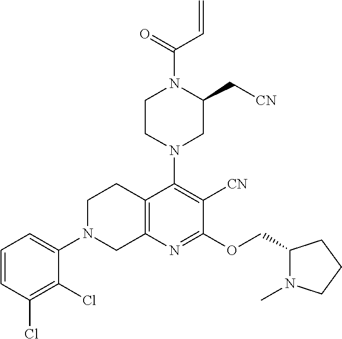 [SMILES: C=CC(=O)N1CCN(c2c(C#N)c(OC[C@@H]3CCCN3C)nc3c2CCN(c2cccc(Cl)c2Cl)C3)C[C@@H]1CC#N] | 4-((S)-4-acryloyl-3- (cyanomethyl)piperazin-1-yl)-7-(2,3- dichlorophenyl)-2-(((S)-1- methylpyrrolidin-2-yl)methoxy)- 5,6,7,8-tetrahydro-1,7-naphthyridine- 3-carbonitrile; | 594 |
| 204 | 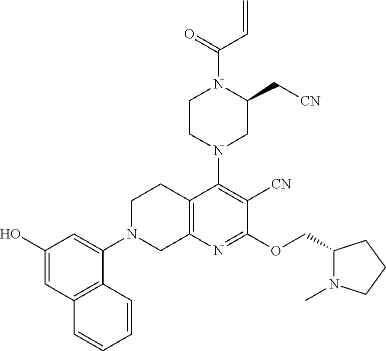 [SMILES: C=CC(=O)N1CCN(c2c(C#N)c(OC[C@@H]3CCCN3C)nc3c2CCN(c2cc(O)cc4ccccc24)C3)C[C@@H]1CC#N] | 4-((S)-4-acryloyl-3- (cyanomethyl)piperazin-1-yl)-7-(3- hydroxynaphthalen-1-yl)-2-(((S)-1- methylpyrrolidin-2-yl)methoxy)- 5,6,7,8-tetrahydro-1,7-naphthyridine- 3-carbonitrile; | 592 |
| 205 | 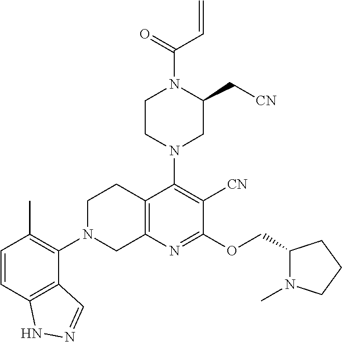 [SMILES: C=CC(=O)N1CCN(c2c(C#N)c(OC[C@@H]3CCCN3C)nc3c2CCN(c2c(C)ccc4[nH]ncc24)C3)C[C@@H]1CC#N] | 4-((S)-4-acryloyl-3- (cyanomethyl)piperazin-1-yl)-7-(5- methyl-1H-indazol-4-yl)-2-(((S)-1- methylpyrrolidin-2-yl)methoxy)- 5,6,7,8-tetrahydro-1,7-naphthyridine- 3-carbonitrile; | 580 |
| 206 | 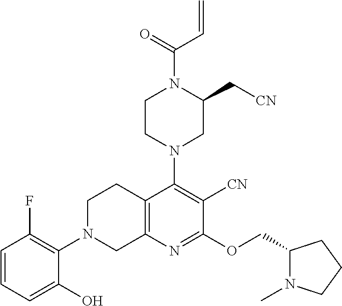 [SMILES: C=CC(=O)N1CCN(c2c(C#N)c(OC[C@@H]3CCCN3C)nc3c2CCN(c2c(O)cccc2F)C3)C[C@@H]1CC#N] | 4-((S)-4-acryloyl-3- (cyanomethyl)piperazin-1-yl)-7-(2- fluoro-6-hydroxyphenyl)-2-(((S)-1- methylpyrrolidin-2-yl)methoxy)- 5,6,7,8-tetrahydro-1,7-naphthyridine- 3-carbonitrile; | 560 |
| 207 | 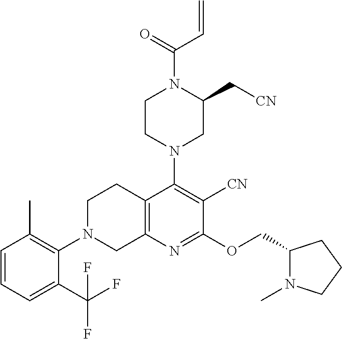 [SMILES: C=CC(=O)N1CCN(c2c(C#N)c(OC[C@@H]3CCCN3C)nc3c2CCN(c2c(C)cccc2C(F)(F)F)C3)C[C@@H]1CC#N] | 4-((S)-4-acryloyl-3- (cyanomethyl)piperazin-1-yl)-7-(2- methyl-6-(trifluoromethyl)phenyl)-2- (((S)-1-methylpyrrolidin-2- yl)methoxy)-5,6,7,8-tetrahydro-1,7- naphthyridine-3-carbonitrile; | 608 |
| 208 | 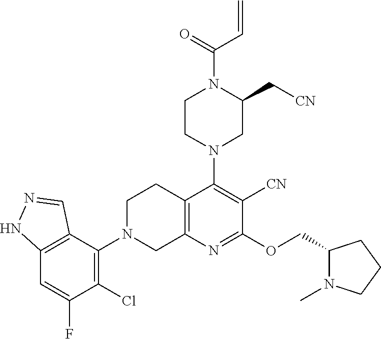 [SMILES: C=CC(=O)N1CCN(c2c(C#N)c(OC[C@@H]3CCCN3C)nc3c2CCN(c2c(Cl)c(F)cc4[nH]ncc24)C3)C[C@@H]1CC#N] | 4-((S)-4-acryloyl-3- (cyanomethyl)piperazin-1-yl)-7-(5- chloro-6-fluoro-1H-indazol-4-yl)-2- (((S)-1-methylpyrrolidin-2- yl)methoxy)-5,6,7,8-tetrahydro-1,7- naphthyridine-3-carbonitrile | 618 |
| 209 | 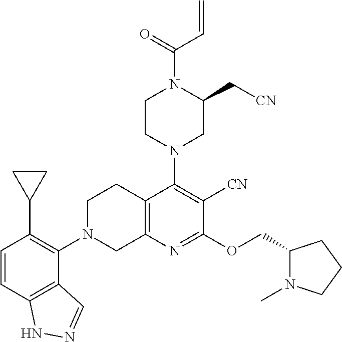 [SMILES: C=CC(=O)N1CCN(c2c(C#N)c(OC[C@@H]3CCCN3C)nc3c2CCN(c2c(C4CC4)ccc4[nH]ncc24)C3)C[C@@H]1CC#N] | 4-((S)-4-acryloyl-3- (cyanomethyl)piperazin-1-yl)-7-(5- cyclopropyl-1H-indazol-4-yl)-2-(((S)- 1-methylpyrrolidin-2-yl)methoxy)- 5,6,7,8-tetrahydro-1,7-naphthyridine- 3-carbonitrile; | 606 |
| 210 | 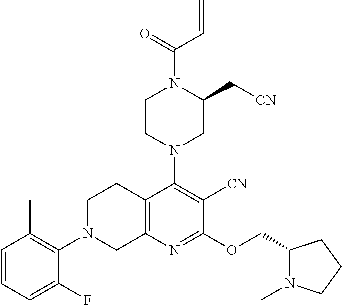 [SMILES: C=CC(=O)N1CCN(c2c(C#N)c(OC[C@@H]3CCCN3C)nc3c2CCN(c2c(C)cccc2F)C3)C[C@@H]1CC#N] | 4-((S)-4-acryloyl-3- (cyanomethyl)piperazin-1-yl)-7-(2- fluoro-6-methylphenyl)-2-(((S)-1- methylpyrrolidin-2-yl)methoxy)- 5,6,7,8-tetrahydro-1,7-naphthyridine- 3-carbonitrile; | 558 |
| 211 | 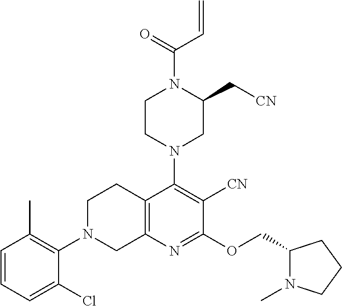 [SMILES: C=CC(=O)N1CCN(c2c(C#N)c(OC[C@@H]3CCCN3C)nc3c2CCN(c2c(C)cccc2Cl)C3)C[C@@H]1CC#N] | 4-((S)-4-acryloyl-3- (cyanomethyl)piperazin-1-yl)-7-(2- chloro-6-methylphenyl)-2-(((S)-1- methylpyrrolidin-2-yl)methoxy)- 5,6,7,8-tetrahydro-1,7-naphthyridine- 3-carbonitrile; | 574 |
| 212 | 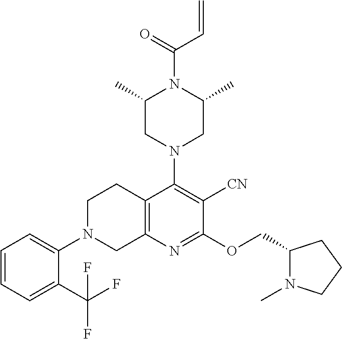 [SMILES: C=CC(=O)N1[C@H](C)CN(c2c(C#N)c(OC[C@@H]3CCCN3C)nc3c2CCN(c2ccccc2C(F)(F)F)C3)C[C@@H]1C] | 4-((3S,5R)-4-acryloyl-3,5- dimethylpiperazin-1-yl)-2-(((S)-1- methylpyrrolidin-2-yl)methoxy)-7-(2- (trifluoromethyl)phenyl)-5,6,7,8- tetrahydro-1,7-naphthyridine-3- carbonitrile; | 583 |
| 213 | 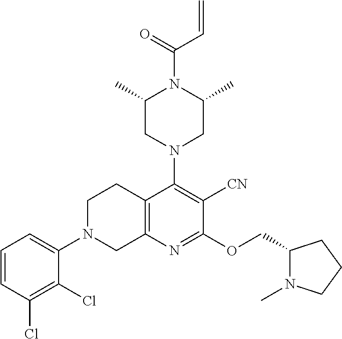 [SMILES: C=CC(=O)N1[C@H](C)CN(c2c(C#N)c(OC[C@@H]3CCCN3C)nc3c2CCN(c2cccc(Cl)c2Cl)C3)C[C@@H]1C] | 4-((3S,5R)-4-acryloyl-3,5- dimethylpiperazin-1-yl)-7-(2,3- dichlorophenyl)-2-(((S)-1- methylpyrrolidin-2-yl)methoxy)- 5,6,7,8-tetrahydro-1,7-naphthyridine- 3-carbonitrile; | 583 |
| 214 | 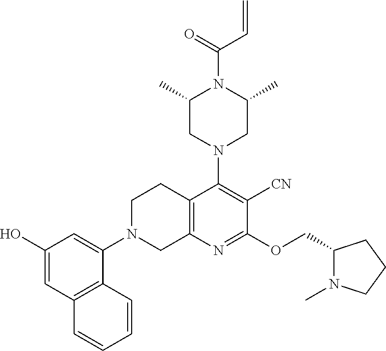 [SMILES: C=CC(=O)N1[C@H](C)CN(c2c(C#N)c(OC[C@@H]3CCCN3C)nc3c2CCN(c2cc(O)cc4ccccc24)C3)C[C@@H]1C] | 4-((3S,5R)-4-acryloyl-3,5- dimethylpiperazin-1-yl)-7-(3- hydroxynaphthalen-1-yl)-2-(((S)-1- methylpyrrolidin-2-yl)methoxy)- 5,6,7,8-tetrahydro-1,7-naphthyridine- 3-carbonitrile; | 581 |
| 215 | 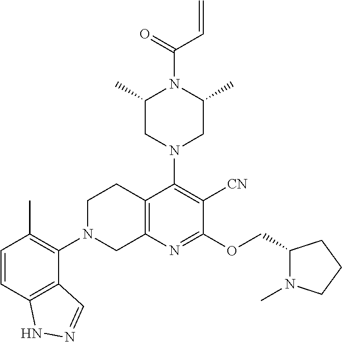 [SMILES: C=CC(=O)N1[C@H](C)CN(c2c(C#N)c(OC[C@@H]3CCCN3C)nc3c2CCN(c2c(C)ccc4[nH]ncc24)C3)C[C@@H]1C] | 4-((3S,5R)-4-acryloyl-3,5- dimethylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 569 |
| 216 | 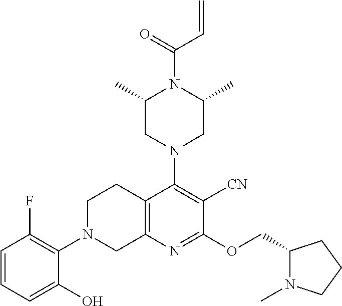 [SMILES: C=CC(=O)N1[C@H](C)CN(c2c(C#N)c(OC[C@@H]3CCCN3C)nc3c2CCN(c2c(O)cccc2F)C3)C[C@@H]1C] | 4-((3S,5R)-4-acryloyl-3,5- dimethylpiperazin-1-yl)-7-(2-fluoro-6- hydroxyphenyl)-2-(((S)-1- methylpyrrolidin-2-yl)methoxy)- 5,6,7,8-tetrahydro-1,7-naphthyridine- 3-carbonitrile; | 549 |
| 217 | 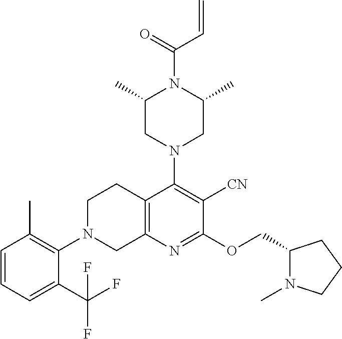 [SMILES: C=CC(=O)N1[C@H](C)CN(c2c(C#N)c(OC[C@@H]3CCCN3C)nc3c2CCN(c2c(C)cccc2C(F)(F)F)C3)C[C@@H]1C] | 4-((3S,5R)-4-acryloyl-3,5- dimethylpiperazin-1-yl)-7-(2-methyl- 6-(trifluoromethyl)phenyl)-2-(((S)-1- methylpyrrolidin-2-yl)methoxy)- 5,6,7,8-tetrahydro-1,7-naphthyridine- 3-carbonitrile; | 597 |
| 218 | 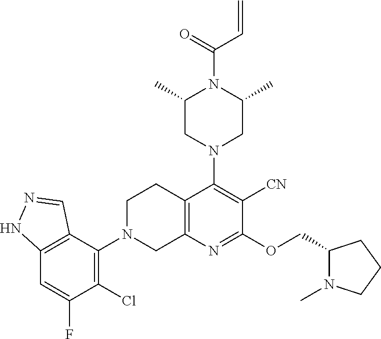 [SMILES: C=CC(=O)N1[C@H](C)CN(c2c(C#N)c(OC[C@@H]3CCCN3C)nc3c2CCN(c2c(Cl)c(F)cc4[nH]ncc24)C3)C[C@@H]1C] | 4-((3S,5R)-4-acryloyl-3,5- dimethylpiperazin-1-yl)-7-(5-chloro- 6-fluoro-1H-indazol-4-yl)-2-(((S)-1- methylpyrrolidin-2-yl)methoxy)- 5,6,7,8-tetrahydro-1,7-naphthyridine- 3-carbonitrile | 607 |
| 219 | 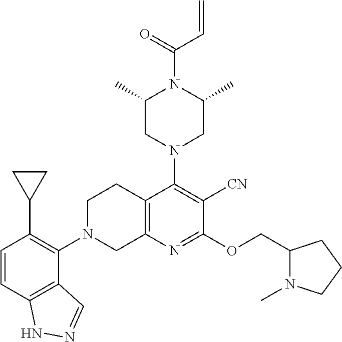 [SMILES: C=CC(=O)N1[C@H](C)CN(c2c(C#N)c(OCC3CCCN3C)nc3c2CCN(c2c(C4CC4)ccc4[nH]ncc24)C3)C[C@@H]1C] | 4-((3S,5R)-4-acryloyl-3,5- dimethylpiperazin-1-yl)-7-(5- cyclopropyl-1H-indazol-4-yl)-2-((1- methylpyrrolidin-2-yl)methoxy)- 5,6,7,8-tetrahydro-1,7-naphthyridine- 3-carbonitrile; | 595 |
| 220 | 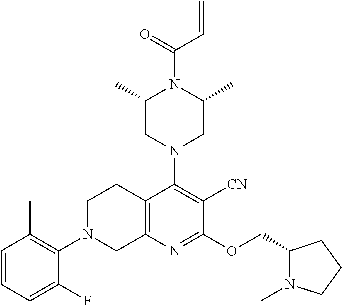 [SMILES: C=CC(=O)N1[C@H](C)CN(c2c(C#N)c(OC[C@@H]3CCCN3C)nc3c2CCN(c2c(C)cccc2F)C3)C[C@@H]1C] | 4-((3S,5R)-4-acryloyl-3,5- dimethylpiperazin-1-yl)-7-(2-fluoro-6- methylphenyl)-2-(((S)-1- methylpyrrolidin-2-yl)methoxy)- 5,6,7,8-tetrahydro-1,7-naphthyridine- 3-carbonitrile; | 547 |
| 221 | 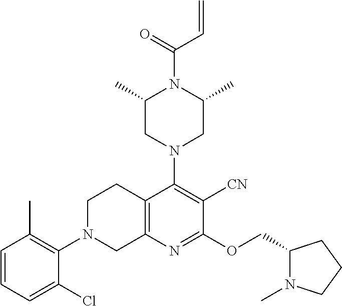 [SMILES: C=CC(=O)N1[C@H](C)CN(c2c(C#N)c(OC[C@@H]3CCCN3C)nc3c2CCN(c2c(C)cccc2Cl)C3)C[C@@H]1C] | 4-((3S,5R)-4-acryloyl-3,5- dimethylpiperazin-1-yl)-7-(2-chloro- 6-methylphenyl)-2-(((S)-1- methylpyrrolidin-2-yl)methoxy)- 5,6,7,8-tetrahydro-1,7-naphthyridine- 3-carbonitrile; | 563 |
| 222 | 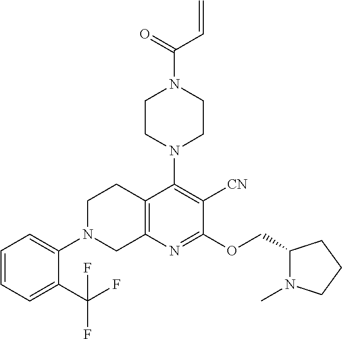 [SMILES: C=CC(=O)N1CCN(c2c(C#N)c(OC[C@@H]3CCCN3C)nc3c2CCN(c2ccccc2C(F)(F)F)C3)CC1] | (S)-4-(4-acryloylpiperazin-1-yl)-2-((1- methylpyrrolidin-2-yl)methoxy)-7-(2- (trifluoromethyl)phenyl)-5,6,7,8- tetrahydro-1,7-naphthyridine-3- carbonitrile; | 555 |
| 223 | 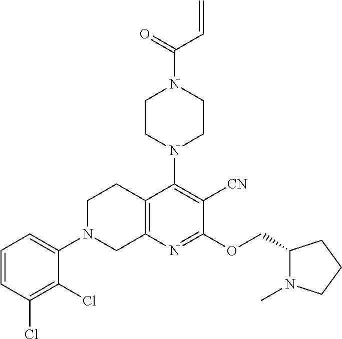 [SMILES: C=CC(=O)N1CCN(c2c(C#N)c(OC[C@@H]3CCCN3C)nc3c2CCN(c2cccc(Cl)c2Cl)C3)CC1] | (S)-4-(4-acryloylpiperazin-1-yl)-7- (2,3-dichlorophenyl)-2-((1- methylpyrrolidin-2-yl)methoxy)- 5,6,7,8-tetrahydro-1,7-naphthyridine- 3-carbonitrile; | 555 |
| 224 | 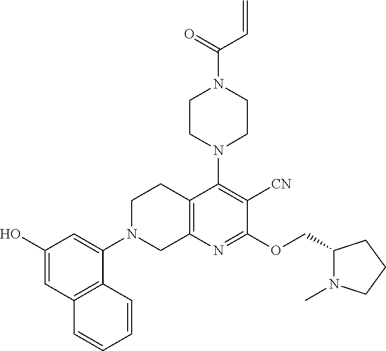 [SMILES: C=CC(=O)N1CCN(c2c(C#N)c(OC[C@@H]3CCCN3C)nc3c2CCN(c2cc(O)cc4ccccc24)C3)CC1] | (S)-4-(4-acryloylpiperazin-1-yl)-7-(3- hydroxynaphthalen-1-yl)-2-((1- methylpyrrolidin-2-yl)methoxy)- 5,6,7,8-tetrahydro-1,7-naphthyridine- 3-carbonitrile; | 553 |
| 225 | 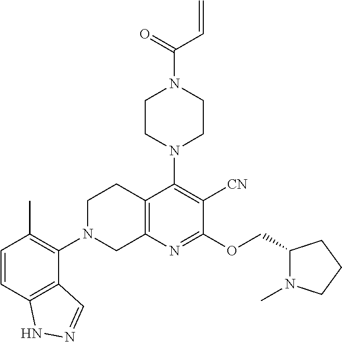 [SMILES: C=CC(=O)N1CCN(c2c(C#N)c(OC[C@@H]3CCCN3C)nc3c2CCN(c2c(C)ccc4[nH]ncc24)C3)CC1] | (S)-4-(4-acryloylpiperazin-1-yl)-7-(5- methyl-1H-indazol-4-yl)-2-((1- methylpyrrolidin-2-yl)methoxy)- 5,6,7,8-tetrahydro-1,7-naphthyridine- 3-carbonitrile; | 541 |
| 226 | 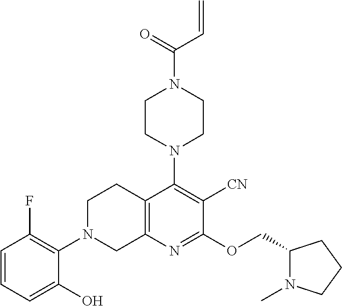 [SMILES: C=CC(=O)N1CCN(c2c(C#N)c(OC[C@@H]3CCCN3C)nc3c2CCN(c2c(O)cccc2F)C3)CC1] | (S)-4-(4-acryloylpiperazin-1-yl)-7-(2- fluoro-6-hydroxyphenyl)-2-((1- methylpyrrolidin-2-yl)methoxy)- 5,6,7,8-tetrahydro-1,7-naphthyridine- 3-carbonitrile; | 521 |
| 227 | 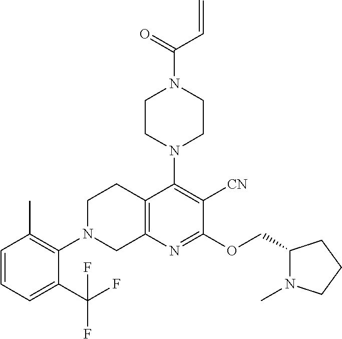 [SMILES: C=CC(=O)N1CCN(c2c(C#N)c(OC[C@@H]3CCCN3C)nc3c2CCN(c2c(C)cccc2C(F)(F)F)C3)CC1] | (S)-4-(4-acryloylpiperazin-1-yl)-7-(2- methyl-6-(trifluoromethyl)phenyl)-2- ((1-methylpyrrolidin-2-yl)methoxy)- 5,6,7,8-tetrahydro-1,7-naphthyridine- 3-carbonitrile; | 569 |
| 228 | 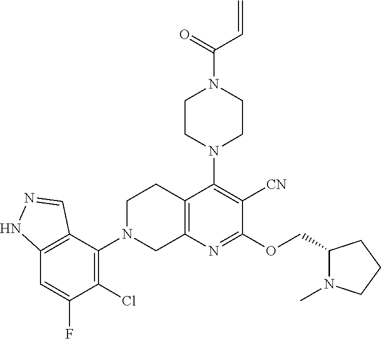 [SMILES: C=CC(=O)N1CCN(c2c(C#N)c(OC[C@@H]3CCCN3C)nc3c2CCN(c2c(Cl)c(F)cc4[nH]ncc24)C3)CC1] | (S)-4-(4-acryloylpiperazin-1-yl)-7-(5- chloro-6-fluoro-1H-indazol-4-yl)-2- ((1-methylpyrrolidin-2-yl)methoxy)- 5,6,7,8-tetrahydro-1,7-naphthyridine- 3-carbonitrile | 579 |
| 229 | 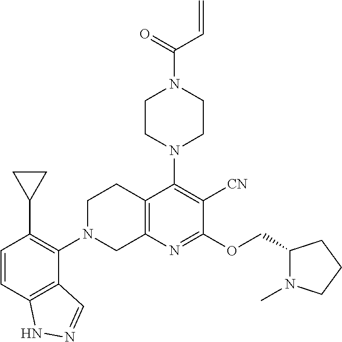 [SMILES: C=CC(=O)N1CCN(c2c(C#N)c(OC[C@@H]3CCCN3C)nc3c2CCN(c2c(C4CC4)ccc4[nH]ncc24)C3)CC1] | (S)-4-(4-acryloylpiperazin-1-yl)-7-(5- cyclopropyl-1H-indazol-4-yl)-2-((1- methylpyrrolidin-2-yl)methoxy)- 5,6,7,8-tetrahydro-1,7-naphthyridine- 3-carbonitrile; | 567 |
| 230 | 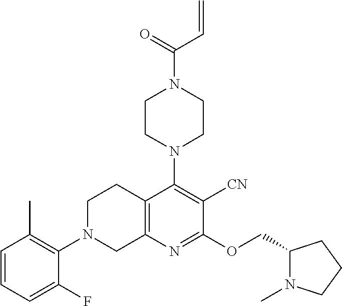 [SMILES: C=CC(=O)N1CCN(c2c(C#N)c(OC[C@@H]3CCCN3C)nc3c2CCN(c2c(C)cccc2F)C3)CC1] | (S)-4-(4-acryloylpiperazin-1-yl)-7-(2- fluoro-6-methylphenyl)-2-((1- methylpyrrolidin-2-yl)methoxy)- 5,6,7,8-tetrahydro-1,7-naphthyridine- 3-carbonitrile; | 519 |
| 231 | 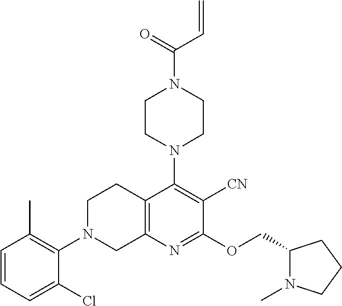 [SMILES: C=CC(=O)N1CCN(c2c(C#N)c(OC[C@@H]3CCCN3C)nc3c2CCN(c2c(C)cccc2Cl)C3)CC1] | (S)-4-(4-acryloylpiperazin-1-yl)-7-(2- chloro-6-methylphenyl)-2-((1- methylpyrrolidin-2-yl)methoxy)- 5,6,7,8-tetrahydro-1,7-naphthyridine- 3-carbonitrile; | 535 |
| 232 | 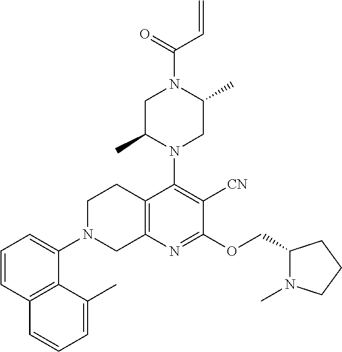 [SMILES: C=CC(=O)N1C[C@H](C)N(c2c(C#N)c(OC[C@@H]3CCCN3C)nc3c2CCN(c2cccc4cccc(C)c24)C3)C[C@H]1C] | 4-((2S,5R)-4-acryloyl-2,5- dimethylpiperazin-1-yl)-7-(8- methylnaphthalen-1-yl)-2-(((S)-1- methylpyrrolidin-2-yl)methoxy)- 5,6,7,8-tetrahydro-1,7-naphthyridine- 3-carbonitrile; | 579 |
| 233 | 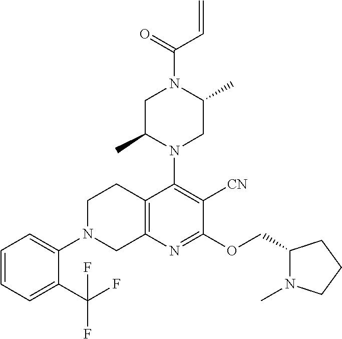 [SMILES: C=CC(=O)N1C[C@H](C)N(c2c(C#N)c(OC[C@@H]3CCCN3C)nc3c2CCN(c2ccccc2C(F)(F)F)C3)C[C@H]1C] | 4-((2S,5R)-4-acryloyl-2,5- dimethylpiperazin-1-yl)-2-(((S)-1- methylpyrrolidin-2-yl)methoxy)-7-(2- (trifluoromethyl)phenyl)-5,6,7,8- tetrahydro-1,7-naphthyridine-3- carbonitrile; | 583 |
| 234 | 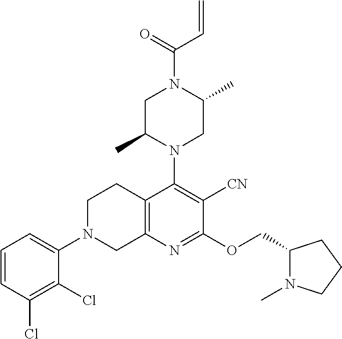 [SMILES: C=CC(=O)N1C[C@H](C)N(c2c(C#N)c(OC[C@@H]3CCCN3C)nc3c2CCN(c2cccc(Cl)c2Cl)C3)C[C@H]1C] | 4-((2S,5R)-4-acryloyl-2,5- dimethylpiperazin-1-yl)-7-(2,3- dichlorophenyl)-2-(((S)-1- methylpyrrolidin-2-yl)methoxy)- 5,6,7,8-tetrahydro-1,7-naphthyridine- 3-carbonitrile; | 583 |
| 235 | 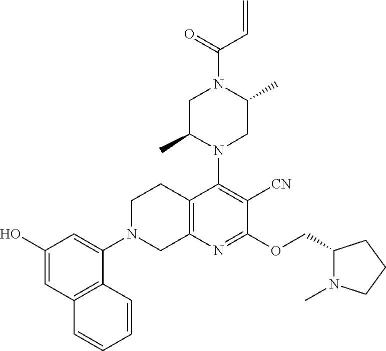 [SMILES: C=CC(=O)N1C[C@H](C)N(c2c(C#N)c(OC[C@@H]3CCCN3C)nc3c2CCN(c2cc(O)cc4ccccc24)C3)C[C@H]1C] | 4-((2S,5R)-4-acryloyl-2,5- dimethylpiperazin-1-yl)-7-(3- hydroxynaphthalen-1-yl)-2-(((S)-1- methylpyrrolidin-2-yl)methoxy)- 5,6,7,8-tetrahydro-1,7-naphthyridine- 3-carbonitrile; | 581 |
| 236 | 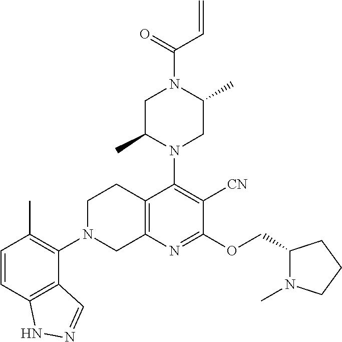 [SMILES: C=CC(=O)N1C[C@H](C)N(c2c(C#N)c(OC[C@@H]3CCCN3C)nc3c2CCN(c2c(C)ccc4[nH]ncc24)C3)C[C@H]1C] | 4-((2S,5R)-4-acryloyl-2,5- dimethylpiperazin-1-yl)-7-(5-methyl- 1H-indazol-4-yl)-2-(((S)-1- methylpyrrolidin-2-yl)methoxy)- 5,6,7,8-tetrahydro-1,7-naphthyridine- 3-carbonitrile; | 569 |
| 237 | 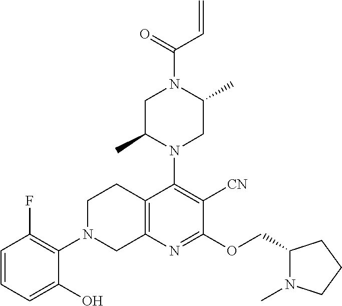 [SMILES: C=CC(=O)N1C[C@H](C)N(c2c(C#N)c(OC[C@@H]3CCCN3C)nc3c2CCN(c2c(O)cccc2F)C3)C[C@H]1C] | 4-((2S,5R)-4-acryloyl-2,5- dimethylpiperazin-1-yl)-7-(2-fluoro-6- hydroxyphenyl)-2-(((S)-1- methylpyrrolidin-2-yl)methoxy)- 5,6,7,8-tetrahydro-1,7-naphthyridine- 3-carbonitrile; | 549 |
| 238 | 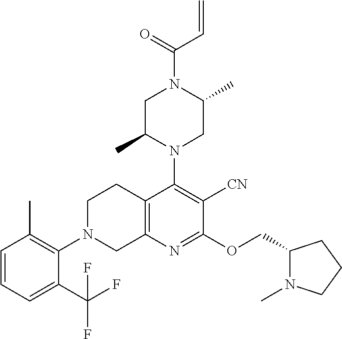 [SMILES: C=CC(=O)N1C[C@H](C)N(c2c(C#N)c(OC[C@@H]3CCCN3C)nc3c2CCN(c2c(C)cccc2C(F)(F)F)C3)C[C@H]1C] | 4-((2S,5R)-4-acryloyl-2,5- dimethylpiperazin-1-yl)-7-(2-methyl- 6-(trifluoromethyl)phenyl)-2-(((S)-1- methylpyrrolidin-2-yl)methoxy)- 5,6,7,8-tetrahydro-1,7-naphthyridine- 3-carbonitrile; | 597 |
| 239 | 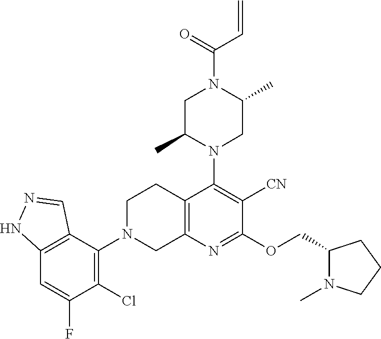 [SMILES: C=CC(=O)N1C[C@H](C)N(c2c(C#N)c(OC[C@@H]3CCCN3C)nc3c2CCN(c2c(Cl)c(F)cc4[nH]ncc24)C3)C[C@H]1C] | 4-((2S,5R)-4-acryloyl-2,5- dimethylpiperazin-1-yl)-7-(5-chloro- 6-fluoro-1H-indazol-4-yl)-2-(((S)-1- methylpyrrolidin-2-yl)methoxy)- 5,6,7,8-tetrahydro-1,7-naphthyridine- 3-carbonitrile | 607 |
| 240 | 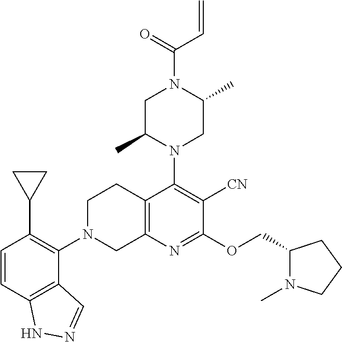 [SMILES: C=CC(=O)N1C[C@H](C)N(c2c(C#N)c(OC[C@@H]3CCCN3C)nc3c2CCN(c2c(C4CC4)ccc4[nH]ncc24)C3)C[C@H]1C] | 4-((2S,5R)-4-acryloyl-2,5- dimethylpiperazin-1-yl)-7-(5- cyclopropyl-1H-indazol-4-yl)-2-(((S)- 1-methylpyrrolidin-2-yl)methoxy)- 5,6,7,8-tetrahydro-1,7-naphthyridine- 3-carbonitrile; | 595 |
| 241 | 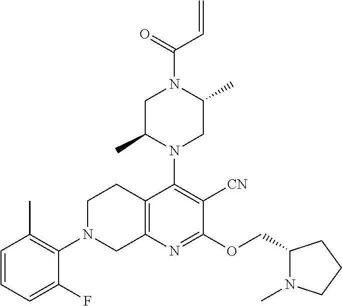 [SMILES: C=CC(=O)N1C[C@H](C)N(c2c(C#N)c(OC[C@@H]3CCCN3C)nc3c2CCN(c2c(C)cccc2F)C3)C[C@H]1C] | 4-((2S,5R)-4-acryloyl-2,5- dimethylpiperazin-1-yl)-7-(2-fluoro-6- methylphenyl)-2-(((S)-1- methylpyrrolidin-2-yl)methoxy)- 5,6,7,8-tetrahydro-1,7-naphthyridine- 3-carbonitrile; | 547 |
| 242 | 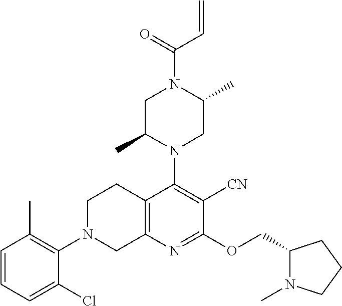 [SMILES: C=CC(=O)N1C[C@H](C)N(c2c(C#N)c(OC[C@@H]3CCCN3C)nc3c2CCN(c2c(C)cccc2Cl)C3)C[C@H]1C] | 4-((2S,5R)-4-acryloyl-2,5- dimethylpiperazin-1-yl)-7-(2-chloro- 6-methylphenyl)-2-(((S)-1- methylpyrrolidin-2-yl)methoxy)- 5,6,7,8-tetrahydro-1,7-naphthyridine- 3-carbonitrile; | 563 |
| 243 | 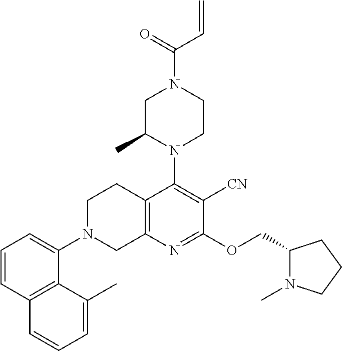 [SMILES: C=CC(=O)N1CCN(c2c(C#N)c(OC[C@@H]3CCCN3C)nc3c2CCN(c2cccc4cccc(C)c24)C3)[C@@H](C)C1] | 4-((S)-4-acryloyl-2-methylpiperazin- 1-yl)-7-(8-methylnaphthalen-1-yl)-2- (((S)-1-methylpyrrolidin-2- yl)methoxy)-5,6,7,8-tetrahydro-1,7- naphthyridine-3-carbonitrile; | 565 |
| 244 | 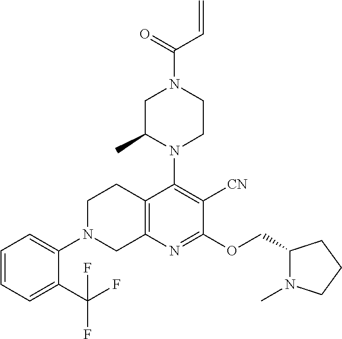 [SMILES: C=CC(=O)N1CCN(c2c(C#N)c(OC[C@@H]3CCCN3C)nc3c2CCN(c2ccccc2C(F)(F)F)C3)[C@@H](C)C1] | 4-((S)-4-acryloyl-2-methylpiperazin- 1-yl)-2-(((S)-1-methylpyrrolidin-2- yl)methoxy)-7-(2- (trifluoromethyl)phenyl)-5,6,7,8- tetrahydro-1,7-naphthyridine-3- carbonitrile; | 569 |
| 245 | 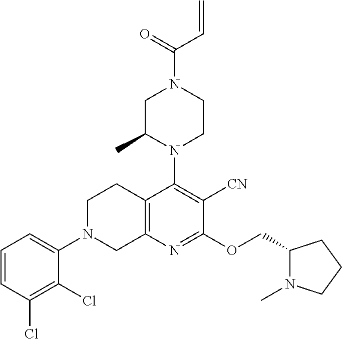 [SMILES: C=CC(=O)N1CCN(c2c(C#N)c(OC[C@@H]3CCCN3C)nc3c2CCN(c2cccc(Cl)c2Cl)C3)[C@@H](C)C1] | 4-((S)-4-acryloyl-2-methylpiperazin- 1-yl)-7-(2,3-dichlorophenyl)-2-(((S)- 1-methylpyrrolidin-2-yl)methoxy)- 5,6,7,8-tetrahydro-1,7-naphthyridine- 3-carbonitrile; | 569 |
| 246 | 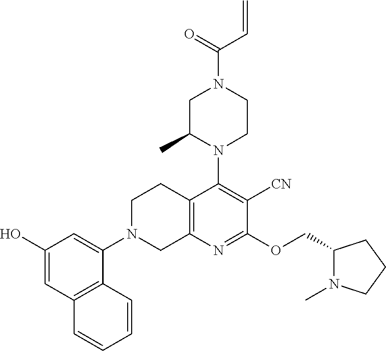 [SMILES: C=CC(=O)N1CCN(c2c(C#N)c(OC[C@@H]3CCCN3C)nc3c2CCN(c2cc(O)cc4ccccc24)C3)[C@@H](C)C1] | 4-((S)-4-acryloyl-2-methylpiperazin- 1-yl)-7-(3-hydroxynaphthalen-1-yl)-2- (((S)-1-methylpyrrolidin-2- yl)methoxy)-5,6,7,8-tetrahydro-1,7- naphthyridine-3-carbonitrile; | 567 |
| 247 | 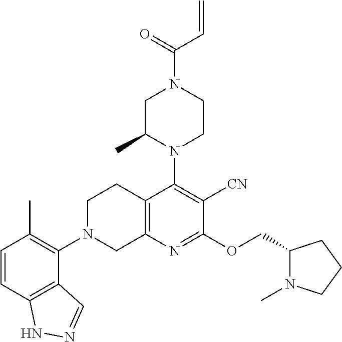 [SMILES: C=CC(=O)N1CCN(c2c(C#N)c(OC[C@@H]3CCCN3C)nc3c2CCN(c2c(C)ccc4[nH]ncc24)C3)[C@@H](C)C1] | 4-((S)-4-acryloyl-2-methylpiperazin- 1-yl)-7-(5-methyl-1H-indazol-4-yl)-2- (((S)-1-methylpyrrolidin-2- yl)methoxy)-5,6,7,8-tetrahydro-1,7- naphthyridine-3-carbonitrile; | 555 |
| 248 | 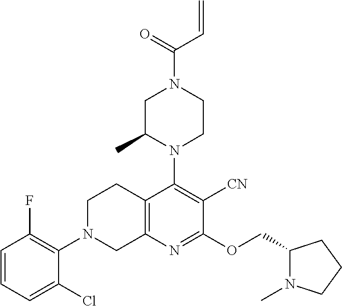 [SMILES: C=CC(=O)N1CCN(c2c(C#N)c(OC[C@@H]3CCCN3C)nc3c2CCN(c2c(F)cccc2Cl)C3)[C@@H](C)C1] | 4-((S)-4-acryloyl-2-methylpiperazin- 1-yl)-7-(2-fluoro-6-hydroxyphenyl)-2- (((S)-1-methylpyrrolidin-2- yl)methoxy)-5,6,7,8-tetrahydro-1,7- naphthyridine-3-carbonitrile; | 535 |
| 249 | 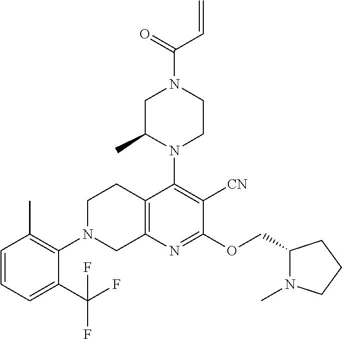 [SMILES: C=CC(=O)N1CCN(c2c(C#N)c(OC[C@@H]3CCCN3C)nc3c2CCN(c2c(C)cccc2C(F)(F)F)C3)[C@@H](C)C1] | 4-((S)-4-acryloyl-2-methylpiperazin- 1-yl)-7-(2-methyl-6- (trifluoromethyl)phenyl)-2-(((S)-1- methylpyrrolidin-2-yl)methoxy)- 5,6,7,8-tetrahydro-1,7-naphthyridine- 3-carbonitrile; | 583 |
| 250 | 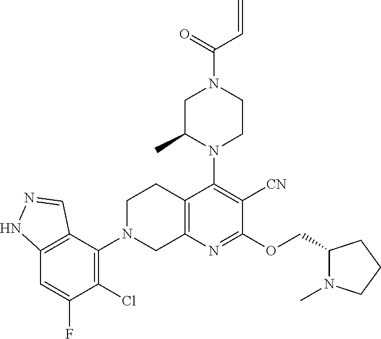 [SMILES: C=CC(=O)N1CCN(c2c(C#N)c(OC[C@@H]3CCCN3C)nc3c2CCN(c2c(Cl)c(F)cc4[nH]ncc24)C3)[C@@H](C)C1] | 4-((S)-4-acryloyl-2-methylpiperazin- 1-yl)-7-(5-chloro-6-fluoro-1H- indazol-4-yl)-2-(((S)-1- methylpyrrolidin-2-yl)methoxy)- 5,6,7,8-tetrahydro-1,7-naphthyridine- 3-carbonitrile | 593 |
| 251 | 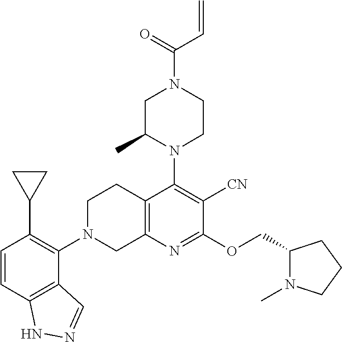 [SMILES: C=CC(=O)N1CCN(c2c(C#N)c(OC[C@@H]3CCCN3C)nc3c2CCN(c2c(C4CC4)ccc4[nH]ncc24)C3)[C@@H](C)C1] | 4-((S)-4-acryloyl-2-methylpiperazin- 1-yl)-7-(5-cyclopropyl-1H-indazol-4- yl)-2-(((S)-1-methylpyrrolidin-2- yl)methoxy)-5,6,7,8-tetrahydro-1,7- naphthyridine-3-carbonitrile; | 581 |
| 252 | 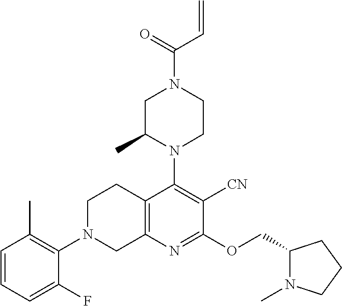 [SMILES: C=CC(=O)N1CCN(c2c(C#N)c(OC[C@@H]3CCCN3C)nc3c2CCN(c2c(C)cccc2F)C3)[C@@H](C)C1] | 4-((S)-4-acryloyl-2-methylpiperazin- 1-yl)-7-(2-fluoro-6-methylphenyl)-2- (((S)-1-methylpyrrolidin-2- yl)methoxy)-5,6,7,8-tetrahydro-1,7- naphthyridine-3-carbonitrile; | 533 |
| 253 | 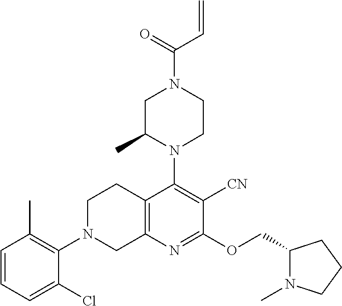 [SMILES: C=CC(=O)N1CCN(c2c(C#N)c(OC[C@@H]3CCCN3C)nc3c2CCN(c2c(C)cccc2Cl)C3)[C@@H](C)C1] | 4-((S)-4-acryloyl-2-methylpiperazin- 1-yl)-7-(2-chloro-6-methylphenyl)-2- (((S)-1-methylpyrrolidin-2- yl)methoxy)-5,6,7,8-tetrahydro-1,7- naphthyridine-3-carbonitrile; | 549 |
| 254 | 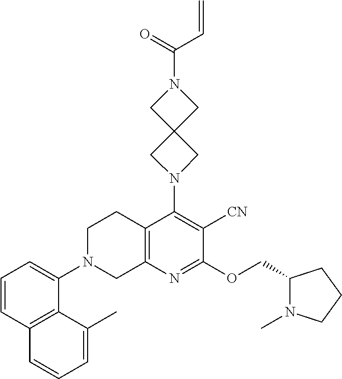 [SMILES: C=CC(=O)N1CC2(C1)CN(c1c(C#N)c(OC[C@@H]3CCCN3C)nc3c1CCN(c1cccc4cccc(C)c14)C3)C2] | (S)-4-(6-acryloyl-2,6- diazaspiro[3.3]heptan-2-yl)-7-(8- methylnaphthalen-1-yl)-2-((1- methylpyrrolidin-2-yl)methoxy)- 5,6,7,8-tetrahydro-1,7-naphthyridine- 3-carbonitrile; | 563 |
| 255 | 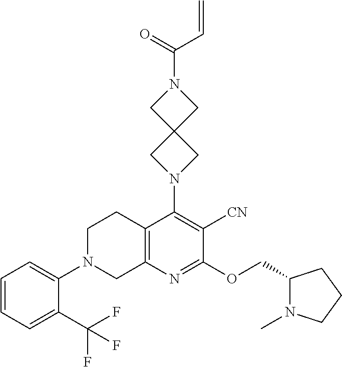 [SMILES: C=CC(=O)N1CC2(C1)CN(c1c(C#N)c(OC[C@@H]3CCCN3C)nc3c1CCN(c1ccccc1C(F)(F)F)C3)C2] | (S)-4-(6-acryloyl-2,6- diazaspiro[3.3]heptan-2-yl)-2-((1- methylpyrrolidin-2-yl)methoxy)-7-(2- (trifluoromethyl)phenyl)-5,6,7,8- tetrahydro-1,7-naphthyridine-3- carbonitrile; | 567 |
| 256 | 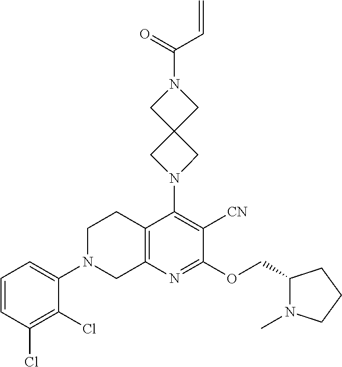 [SMILES: C=CC(=O)N1CC2(C1)CN(c1c(C#N)c(OC[C@@H]3CCCN3C)nc3c1CCN(c1cccc(Cl)c1Cl)C3)C2] | (S)-4-(6-acryloyl-2,6- diazaspiro[3.3]heptan-2-yl)-7-(2,3- dichlorophenyl)-2-((1- methylpyrrolidin-2-yl)methoxy)- 5,6,7,8-tetrahydro-1,7-naphthyridine- 3-carbonitrile; | 567 |
| 257 | 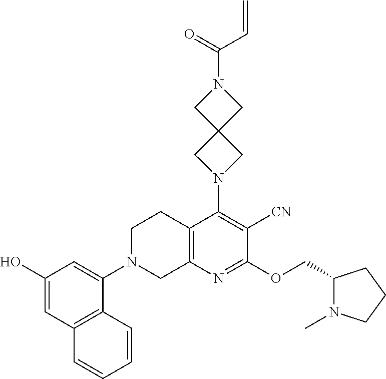 [SMILES: C=CC(=O)N1CC2(C1)CN(c1c(C#N)c(OC[C@@H]3CCCN3C)nc3c1CCN(c1cc(O)cc4ccccc14)C3)C2] | (S)-4-(6-acryloyl-2,6- diazaspiro[3.3]heptan-2-yl)-7-(3- hydroxynaphthalen-1-yl)-2-((1- methylpyrrolidin-2-yl)methoxy)- 5,6,7,8-tetrahydro-1,7-naphthyridine- 3-carbonitrile; | 565 |
| 258 | 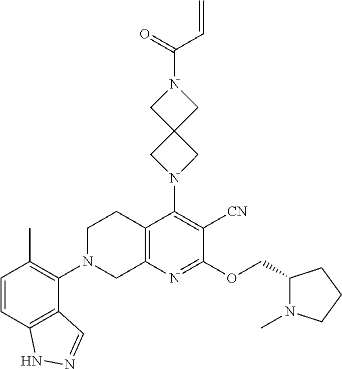 [SMILES: C=CC(=O)N1CC2(C1)CN(c1c(C#N)c(OC[C@@H]3CCCN3C)nc3c1CCN(c1c(C)ccc4[nH]ncc14)C3)C2] | (S)-4-(6-acryloyl-2,6- diazaspiro[3.3]heptan-2-yl)-7-(5- methyl-1H-indazol-4-yl)-2-((1- methylpyrrolidin-2-yl)methoxy)- 5,6,7,8-tetrahydro-1,7-naphthyridine- 3-carbonitrile; | 553 |
| 259 | 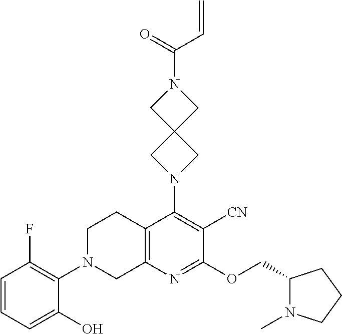 [SMILES: C=CC(=O)N1CC2(C1)CN(c1c(C#N)c(OC[C@@H]3CCCN3C)nc3c1CCN(c1c(O)cccc1F)C3)C2] | (S)-4-(6-acryloyl-2,6- diazaspiro[3.3]heptan-2-yl)-7-(2- fluoro-6-hydroxyphenyl)-2-((1- methylpyrrolidin-2-yl)methoxy)- 5,6,7,8-tetrahydro-1,7-naphthyridine- 3-carbonitrile; | 533 |
| 260 | 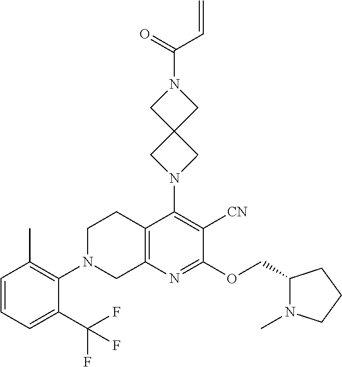 [SMILES: C=CC(=O)N1CC2(C1)CN(c1c(C#N)c(OC[C@@H]3CCCN3C)nc3c1CCN(c1c(C)cccc1C(F)(F)F)C3)C2] | (S)-4-(6-acryloyl-2,6- diazaspiro[3.3]heptan-2-yl)-7-(2- methyl-6-(trifluoromethyl)phenyl)-2- ((1-methylpyrrolidin-2-yl)methoxy)- 5,6,7,8-tetrahydro-1,7-naphthyridine- 3-carbonitrile; | 581 |
| 261 | 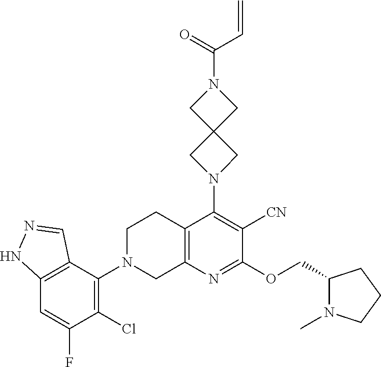 [SMILES: C=CC(=O)N1CC2(C1)CN(c1c(C#N)c(OC[C@@H]3CCCN3C)nc3c1CCN(c1c(Cl)c(F)cc4[nH]ncc14)C3)C2] | (S)-4-(6-acryloyl-2,6- diazaspiro[3.3]heptan-2-yl)-7-(5- chloro-6-fluoro-1H-indazol-4-yl)-2- ((1-methylpyrrolidin-2-yl)methoxy)- 5,6,7,8-tetrahydro-1,7-naphthyridine- 3-carbonitrile | 591 |
| 262 | 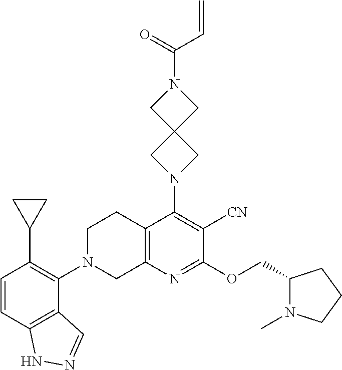 [SMILES: C=CC(=O)N1CC2(C1)CN(c1c(C#N)c(OC[C@@H]3CCCN3C)nc3c1CCN(c1c(C4CC4)ccc4[nH]ncc14)C3)C2] | (S)-4-(6-acryloyl-2,6- diazaspiro[3.3]heptan-2-yl)-7-(5- cyclopropyl-1H-indazol-4-yl)-2-((1- methylpyrrolidin-2-yl)methoxy)- 5,6,7,8-tetrahydro-1,7-naphthyridine- 3-carbonitrile; | 579 |
| 263 | 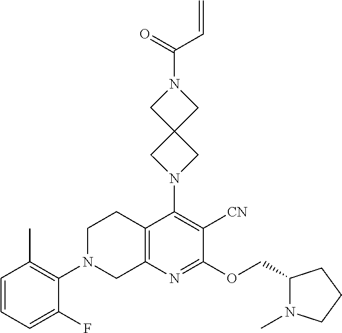 [SMILES: C=CC(=O)N1CC2(C1)CN(c1c(C#N)c(OC[C@@H]3CCCN3C)nc3c1CCN(c1c(C)cccc1F)C3)C2] | (S)-4-(6-acryloyl-2,6- diazaspiro[3.3]heptan-2-yl)-7-(2- fluoro-6-methylphenyl)-2-((1- methylpyrrolidin-2-yl)methoxy)- 5,6,7,8-tetrahydro-1,7-naphthyridine- 3-carbonitrile; | 531 |
| 264 | 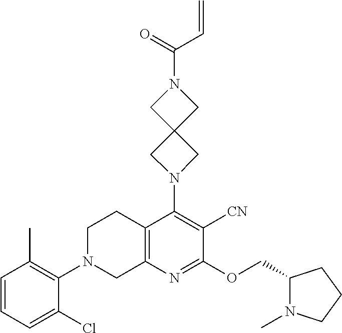 [SMILES: C=CC(=O)N1CC2(C1)CN(c1c(C#N)c(OC[C@@H]3CCCN3C)nc3c1CCN(c1c(C)cccc1Cl)C3)C2] | (S)-4-(6-acryloyl-2,6- diazaspiro[3.3]heptan-2-yl)-7-(2- chloro-6-methylphenyl)-2-((1- methylpyrrolidin-2-yl)methoxy)- 5,6,7,8-tetrahydro-1,7-naphthyridine- 3-carbonitrile; | 547 |
| 265 | 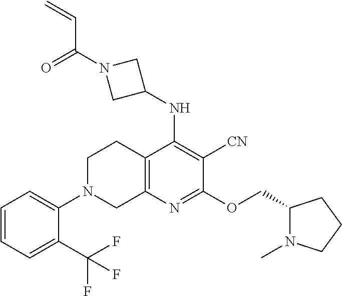 [SMILES: C=CC(=O)N1CC(Nc2c(C#N)c(OC[C@@H]3CCCN3C)nc3c2CCN(c2ccccc2C(F)(F)F)C3)C1] | (S)-4-((1-acryloylazetidin-3- yl)amino)-2-((1-methylpyrrolidin-2- yl)methoxy)-7-(2- (trifluoromethyl)phenyl)-5,6,7,8- tetrahydro-1,7-naphthyridine-3- carbonitrile; | 541 |
| 266 | 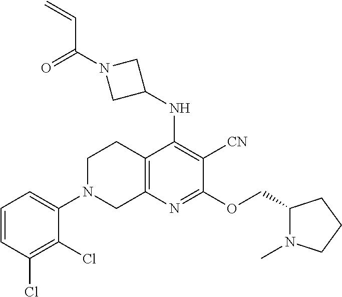 [SMILES: C=CC(=O)N1CC(Nc2c(C#N)c(OC[C@@H]3CCCN3C)nc3c2CCN(c2cccc(Cl)c2Cl)C3)C1] | (S)-4-((1-acryloylazetidin-3- yl)amino)-7-(2,3-dichlorophenyl)-2- ((1-methylpyrrolidin-2-yl)methoxy)- 5,6,7,8-tetrahydro-1,7-naphthyridine- 3-carbonitrile; | 541 |
| 267 | 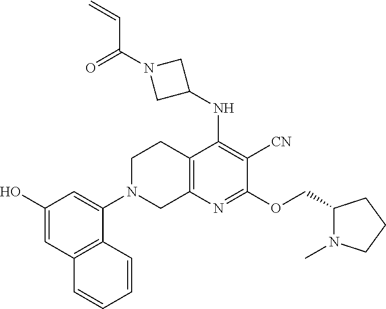 [SMILES: C=CC(=O)N1CC(Nc2c(C#N)c(OC[C@@H]3CCCN3C)nc3c2CCN(c2cc(O)cc4ccccc24)C3)C1] | (S)-4-((1-acryloylazetidin-3- yl)amino)-7-(3-hydroxynaphthalen-1- yl)-2-((1-methylpyrrolidin-2- yl)methoxy)-5,6,7,8-tetrahydro-1,7- naphthyridine-3-carbonitrile; | 539 |
| 268 | 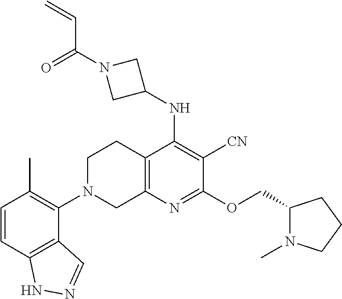 [SMILES: C=CC(=O)N1CC(Nc2c(C#N)c(OC[C@@H]3CCCN3C)nc3c2CCN(c2c(C)ccc4[nH]ncc24)C3)C1] | (S)-4-((1-acryloylazetidin-3- yl)amino)-7-(5-methyl-1H-indazol-4- yl)-2-((1-methylpyrrolidin-2- yl)methoxy)-5,6,7,8-tetrahydro-1,7- naphthyridine-3-carbonitrile; | 527 |
| 269 | 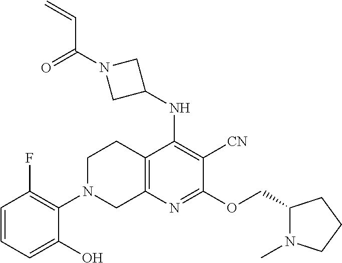 [SMILES: C=CC(=O)N1CC(Nc2c(C#N)c(OC[C@@H]3CCCN3C)nc3c2CCN(c2c(O)cccc2F)C3)C1] | (S)-4-((1-acryloylazetidin-3- yl)amino)-7-(2-fluoro-6- hydroxyphenyl)-2-((1- methylpyrrolidin-2-yl)methoxy)- 5,6,7,8-tetrahydro-1,7-naphthyridine- 3-carbonitrile; | 507 |
| 270 | 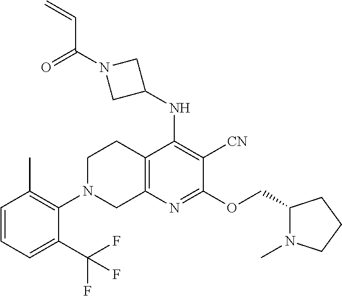 [SMILES: C=CC(=O)N1CC(Nc2c(C#N)c(OC[C@@H]3CCCN3C)nc3c2CCN(c2c(C)cccc2C(F)(F)F)C3)C1] | (S)-4-((1-acryloylazetidin-3- yl)amino)-7-(2-methyl-6- (trifluoromethyl)phenyl)-2-((1- methylpyrrolidin-2-yl)methoxy)- 5,6,7,8-tetrahydro-1,7-naphthyridine- 3-carbonitrile; | 555 |
| 271 | 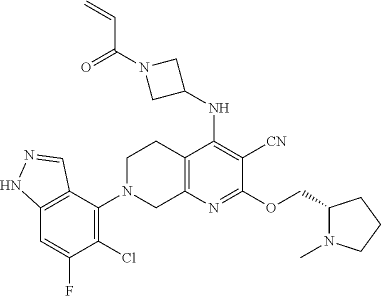 [SMILES: C=CC(=O)N1CC(Nc2c(C#N)c(OC[C@@H]3CCCN3C)nc3c2CCN(c2c(Cl)c(F)cc4[nH]ncc24)C3)C1] | (S)-4-((1-acryloylazetidin-3- yl)amino)-7-(5-chloro-6-fluoro-1H- indazol-4-yl)-2-((1-methylpyrrolidin- 2-yl)methoxy)-5,6,7,8-tetrahydro-1,7- naphthyridine-3-carbonitrile | 565 |
| 272 | 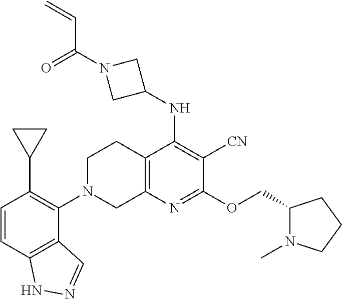 [SMILES: C=CC(=O)N1CC(Nc2c(C#N)c(OC[C@@H]3CCCN3C)nc3c2CCN(c2c(C4CC4)ccc4[nH]ncc24)C3)C1] | (S)-4-((1-acryloylazetidin-3- yl)amino)-7-(5-cyclopropyl-1H- indazol-4-yl)-2-((1-methylpyrrolidin- 2-yl)methoxy)-5,6,7,8-tetrahydro-1,7- naphthyridine-3-carbonitrile; | 553 |
| 273 | 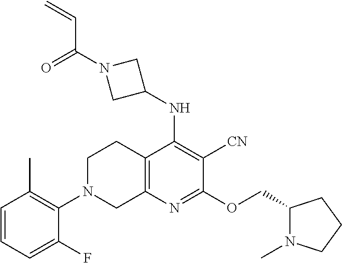 [SMILES: C=CC(=O)N1CC(Nc2c(C#N)c(OC[C@@H]3CCCN3C)nc3c2CCN(c2c(C)cccc2F)C3)C1] | (S)-4-((1-acryloylazetidin-3- yl)amino)-7-(2-fluoro-6- methylphenyl)-2-((1- methylpyrrolidin-2-yl)methoxy)- 5,6,7,8-tetrahydro-1,7-naphthyridine- 3-carbonitrile; | 505 |
| 274 | 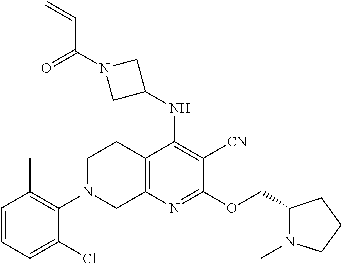 [SMILES: C=CC(=O)N1CC(Nc2c(C#N)c(OC[C@@H]3CCCN3C)nc3c2CCN(c2c(C)cccc2Cl)C3)C1] | (S)-4-((1-acryloylazetidin-3- yl)amino)-7-(2-chloro-6- methylphenyl)-2-((1- methylpyrrolidin-2-yl)methoxy)- 5,6,7,8-tetrahydro-1,7-naphthyridine- 3-carbonitrile; | 521 |
| 275 | 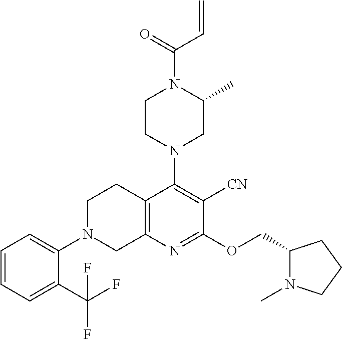 [SMILES: C=CC(=O)N1CCN(c2c(C#N)c(OC[C@@H]3CCCN3C)nc3c2CCN(c2ccccc2C(F)(F)F)C3)C[C@H]1C] | 4-((R)-4-acryloyl-3-methylpiperazin- 1-yl)-2-(((S)-1-methylpyrrolidin-2- yl)methoxy)-7-(2- (trifluoromethyl)phenyl)-5,6,7,8- tetrahydro-1,7-naphthyridine-3- carbonitrile; | 569 |
| 276 | 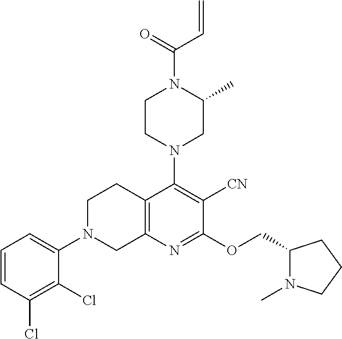 [SMILES: C=CC(=O)N1CCN(c2c(C#N)c(OC[C@@H]3CCCN3C)nc3c2CCN(c2cccc(Cl)c2Cl)C3)C[C@H]1C] | 4-((R)-4-acryloyl-3-methylpiperazin- 1-yl)-7-(2,3-dichlorophenyl)-2-(((S)- 1-methylpyrrolidin-2-yl)methoxy)- 5,6,7,8-tetrahydro-1,7-naphthyridine- 3-carbonitrile; | 569 |
| 277 | 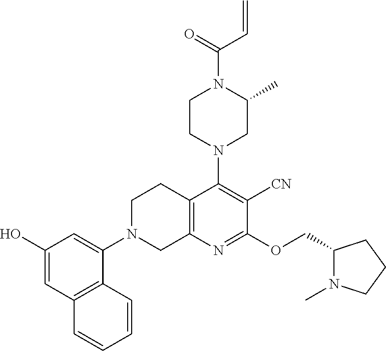 [SMILES: C=CC(=O)N1CCN(c2c(C#N)c(OC[C@@H]3CCCN3C)nc3c2CCN(c2cc(O)cc4ccccc24)C3)C[C@H]1C] | 4-((R)-4-acryloyl-3-methylpiperazin- 1-yl)-7-(3-hydroxynaphthalen-1-yl)-2- (((S)-1-methylpyrrolidin-2- yl)methoxy)-5,6,7,8-tetrahydro-1,7- naphthyridine-3-carbonitrile; | 567 |
| 278 | 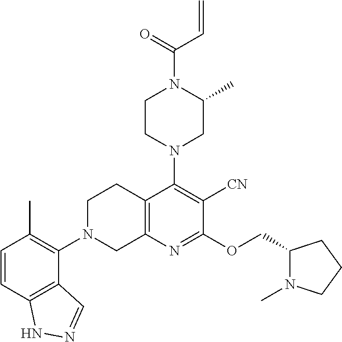 [SMILES: C=CC(=O)N1CCN(c2c(C#N)c(OC[C@@H]3CCCN3C)nc3c2CCN(c2c(C)ccc4[nH]ncc24)C3)C[C@H]1C] | 4-((R)-4-acryloyl-3-methylpiperazin- 1-yl)-7-(5-methyl-1H-indazol-4-yl)-2- (((S)-1-methylpyrrolidin-2- yl)methoxy)-5,6,7,8-tetrahydro-1,7- naphthyridine-3-carbonitrile; | 555 |
| 279 | 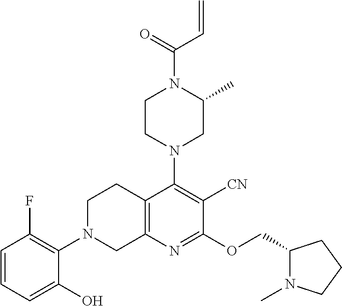 [SMILES: C=CC(=O)N1CCN(c2c(C#N)c(OC[C@@H]3CCCN3C)nc3c2CCN(c2c(O)cccc2F)C3)C[C@H]1C] | 4-((R)-4-acryloyl-3-methylpiperazin- 1-yl)-7-(2-fluoro-6-hydroxyphenyl)-2- (((S)-1-methylpyrrolidin-2- yl)methoxy)-5,6,7,8-tetrahydro-1,7- naphthyridine-3-carbonitrile; | 535 |
| 280 | 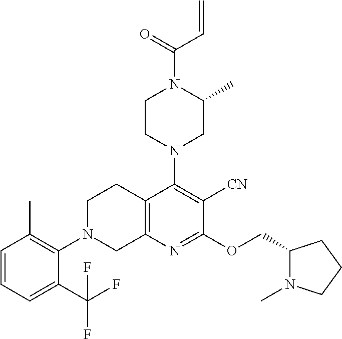 [SMILES: C=CC(=O)N1CCN(c2c(C#N)c(OC[C@@H]3CCCN3C)nc3c2CCN(c2c(C)cccc2C(F)(F)F)C3)C[C@H]1C] | 4-((R)-4-acryloyl-3-methylpiperazin- 1-yl)-7-(2-methyl-6- (trifluoromethyl)phenyl)-2-(((S)-1- methylpyrrolidin-2-yl)methoxy)- 5,6,7,8-tetrahydro-1,7-naphthyridine- 3-carbonitrile; | 583 |
| 281 | 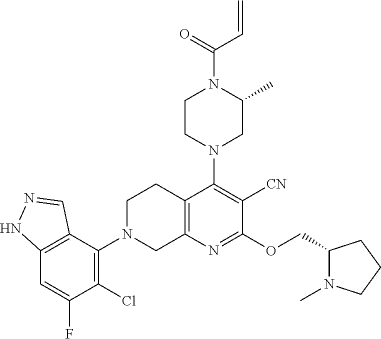 [SMILES: C=CC(=O)N1CCN(c2c(C#N)c(OC[C@@H]3CCCN3C)nc3c2CCN(c2c(Cl)c(F)cc4[nH]ncc24)C3)C[C@H]1C] | 4-((R)-4-acryloyl-3-methylpiperazin- 1-yl)-7-(5-chloro-6-fluoro-1H- indazol-4-yl)-2-(((S)-1- methylpyrrolidin-2-yl)methoxy)- 5,6,7,8-tetrahydro-1,7-naphthyridine- 3-carbonitrile | 593 |
| 282 | 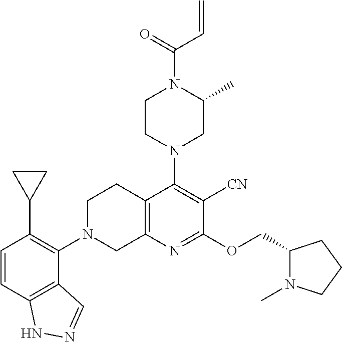 [SMILES: C=CC(=O)N1CCN(c2c(C#N)c(OC[C@@H]3CCCN3C)nc3c2CCN(c2c(C4CC4)ccc4[nH]ncc24)C3)C[C@H]1C] | 4-((R)-4-acryloyl-3-methylpiperazin- 1-yl)-7-(5-cyclopropyl-1H-indazol-4- yl)-2-(((S)-1-methylpyrrolidin-2- yl)methoxy)-5,6,7,8-tetrahydro-1,7- naphthyridine-3-carbonitrile; | 581 |
| 283 | 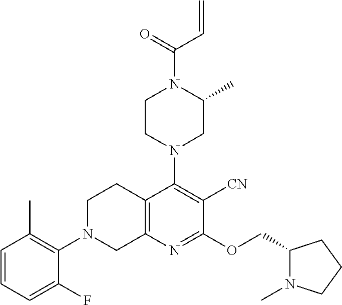 [SMILES: C=CC(=O)N1CCN(c2c(C#N)c(OC[C@@H]3CCCN3C)nc3c2CCN(c2c(C)cccc2F)C3)C[C@H]1C] | 4-((R)-4-acryloyl-3-methylpiperazin- 1-yl)-7-(2-fluoro-6-methylphenyl)-2- (((S)-1-methylpyrrolidin-2- yl)methoxy)-5,6,7,8-tetrahydro-1,7- naphthyridine-3-carbonitrile; | 533 |
| 284 | 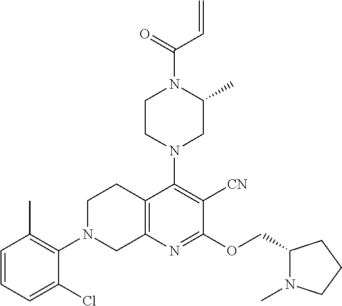 [SMILES: C=CC(=O)N1CCN(c2c(C#N)c(OC[C@@H]3CCCN3C)nc3c2CCN(c2c(C)cccc2Cl)C3)C[C@H]1C] | 4-((R)-4-acryloyl-3-methylpiperazin- 1-yl)-7-(2-chloro-6-methylphenyl)-2- (((S)-1-methylpyrrolidin-2- yl)methoxy)-5,6,7,8-tetrahydro-1,7- naphthyridine-3-carbonitrile; | 549 |
| 285 | 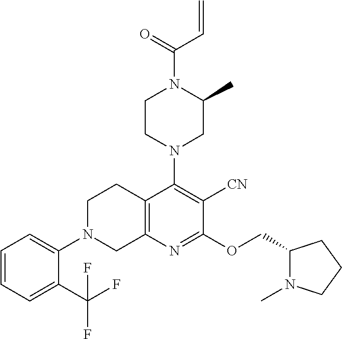 [SMILES: C=CC(=O)N1CCN(c2c(C#N)c(OC[C@@H]3CCCN3C)nc3c2CCN(c2ccccc2C(F)(F)F)C3)C[C@@H]1C] | 4-((S)-4-acryloyl-3-methylpiperazin- 1-yl)-2-(((S)-1-methylpyrrolidin-2- yl)methoxy)-7-(2- (trifluoromethyl)phenyl)-5,6,7,8- tetrahydro-1,7-naphthyridine-3- carbonitrile; | 569 |
| 286 | 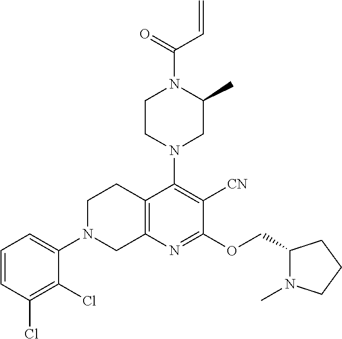 [SMILES: C=CC(=O)N1CCN(c2c(C#N)c(OC[C@@H]3CCCN3C)nc3c2CCN(c2cccc(Cl)c2Cl)C3)C[C@@H]1C] | 4-((S)-4-acryloyl-3-methylpiperazin- 1-yl)-7-(2,3-dichlorophenyl)-2-(((S)- 1-methylpyrrolidin-2-yl)methoxy)- 5,6,7,8-tetrahydro-1,7-naphthyridine- 3-carbonitrile; | 569 |
| 287 | 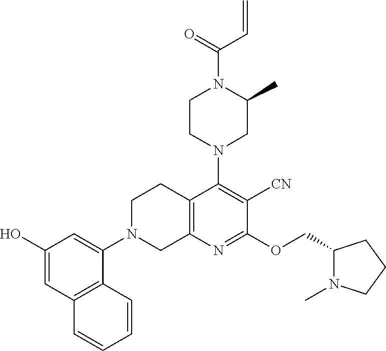 [SMILES: C=CC(=O)N1CCN(c2c(C#N)c(OC[C@@H]3CCCN3C)nc3c2CCN(c2cc(O)cc4ccccc24)C3)C[C@@H]1C] | 4-((S)-4-acryloyl-3-methylpiperazin- 1-yl)-7-(3-hydroxynaphthalen-1-yl)-2- (((S)-1-methylpyrrolidin-2- yl)methoxy)-5,6,7,8-tetrahydro-1,7- naphthyridine-3-carbonitrile; | 567 |
| 288 | 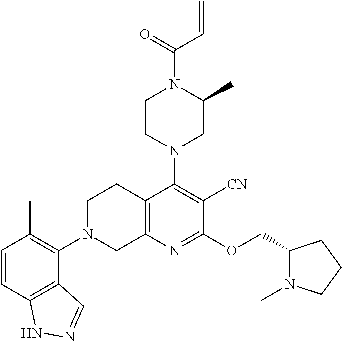 [SMILES: C=CC(=O)N1CCN(c2c(C#N)c(OC[C@@H]3CCCN3C)nc3c2CCN(c2c(C)ccc4[nH]ncc24)C3)C[C@@H]1C] | 4-((S)-4-acryloyl-3-methylpiperazin- 1-yl)-7-(5-methyl-1H-indazol-4-yl)-2- (((S)-1-methylpyrrolidin-2- yl)methoxy)-5,6,7,8-tetrahydro-1,7- naphthyridine-3-carbonitrile; | 555 |
| 289 | 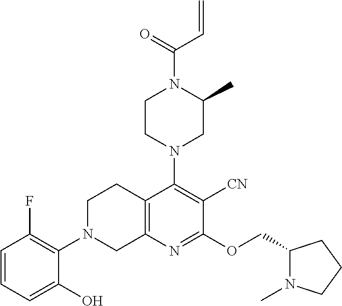 [SMILES: C=CC(=O)N1CCN(c2c(C#N)c(OC[C@@H]3CCCN3C)nc3c2CCN(c2c(O)cccc2F)C3)C[C@@H]1C] | 4-((S)-4-acryloyl-3-methylpiperazin- 1-yl)-7-(2-fluoro-6-hydroxyphenyl)-2- (((S)-1-methylpyrrolidin-2- yl)methoxy)-5,6,7,8-tetrahydro-1,7- naphthyridine-3-carbonitrile; | 535 |
| 290 | 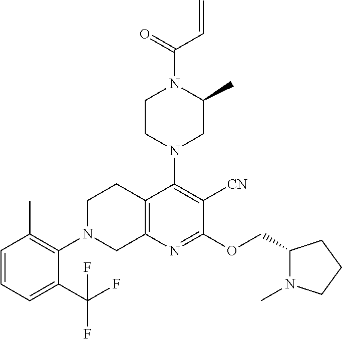 [SMILES: C=CC(=O)N1CCN(c2c(C#N)c(OC[C@@H]3CCCN3C)nc3c2CCN(c2c(C)cccc2C(F)(F)F)C3)C[C@@H]1C] | 4-((S)-4-acryloyl-3-methylpiperazin- 1-yl)-7-(2-methyl-6- (trifluoromethyl)phenyl)-2-(((S)-1- methylpyrrolidin-2-yl)methoxy)- 5,6,7,8-tetrahydro-1,7-naphthyridine- 3-carbonitrile; | 583 |
| 291 | 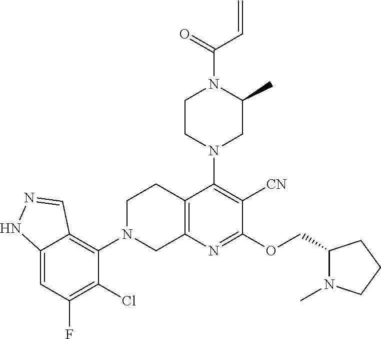 [SMILES: C=CC(=O)N1CCN(c2c(C#N)c(OC[C@@H]3CCCN3C)nc3c2CCN(c2c(Cl)c(F)cc4[nH]ncc24)C3)C[C@@H]1C] | 4-((S)-4-acryloyl-3-methylpiperazin- 1-yl)-7-(5-chloro-6-fluoro-1H- indazol-4-yl)-2-(((S)-1- methylpyrrolidin-2-yl)methoxy)- 5,6,7,8-tetrahydro-1,7-naphthyridine- 3-carbonitrile | 593 |
| 292 | 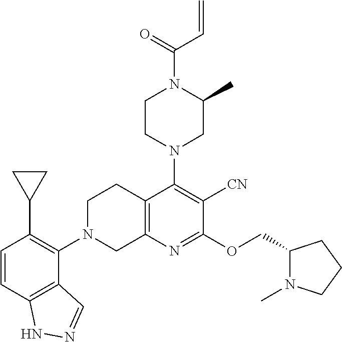 [SMILES: C=CC(=O)N1CCN(c2c(C#N)c(OC[C@@H]3CCCN3C)nc3c2CCN(c2c(C4CC4)ccc4[nH]ncc24)C3)C[C@@H]1C] | 4-((S)-4-acryloyl-3-methylpiperazin- 1-yl)-7-(5-cyclopropyl-1H-indazol-4- yl)-2-(((S)-1-methylpyrrolidin-2- yl)methoxy)-5,6,7,8-tetrahydro-1,7- naphthyridine-3-carbonitrile; | 581 |
| 293 | 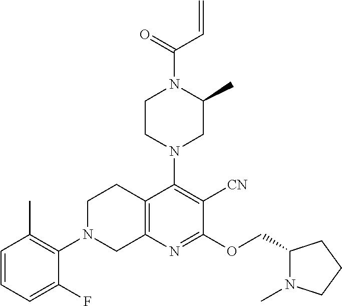 [SMILES: C=CC(=O)N1CCN(c2c(C#N)c(OC[C@@H]3CCCN3C)nc3c2CCN(c2c(C)cccc2F)C3)C[C@@H]1C] | 4-((S)-4-acryloyl-3-methylpiperazin- 1-yl)-7-(2-fluoro-6-methylphenyl)-2- (((S)-1-methylpyrrolidin-2- yl)methoxy)-5,6,7,8-tetrahydro-1,7- naphthyridine-3-carbonitrile; | 533 |
| 294 | 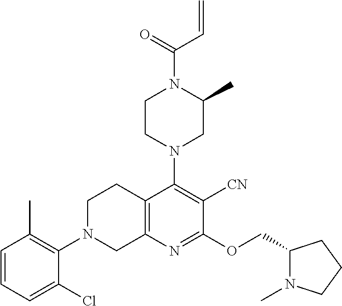 [SMILES: C=CC(=O)N1CCN(c2c(C#N)c(OC[C@@H]3CCCN3C)nc3c2CCN(c2c(C)cccc2Cl)C3)C[C@@H]1C] | 4-((S)-4-acryloyl-3-methylpiperazin- 1-yl)-7-(2-chloro-6-methylphenyl)-2- (((S)-1-methylpyrrolidin-2- yl)methoxy)-5,6,7,8-tetrahydro-1,7- naphthyridine-3-carbonitrile; | 549 |
| 295 | 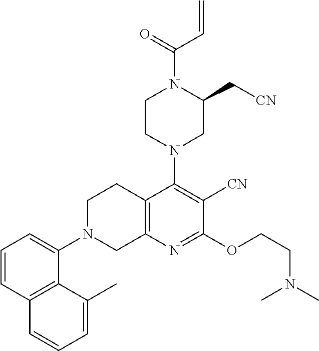 [SMILES: C=CC(=O)N1CCN(c2c(C#N)c(OCCN(C)C)nc3c2CCN(c2cccc4cccc(C)c24)C3)C[C@@H]1CC#N] | (S)-4-(4-acryloyl-3- (cyanomethyl)piperazin-1-yl)-2-(2- (dimethylamino)ethoxy)-7-(8- methylnaphthalen-1-yl)-5,6,7,8- tetrahydro-1,7-naphthyridine-3- carbonitrile; | 564 |
| 296 | 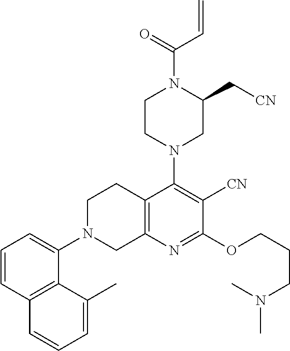 [SMILES: C=CC(=O)N1CCN(c2c(C#N)c(OCCCN(C)C)nc3c2CCN(c2cccc4cccc(C)c24)C3)C[C@@H]1CC#N] | (S)-4-(4-acryloyl-3- (cyanomethyl)piperazin-1-yl)-2-(3- (dimethylamino)propoxy)-7-(8- methylnaphthalen-1-yl)-5,6,7,8- tetrahydro-1,7-naphthyridine-3- carbonitrile; | 578 |
| 297 | 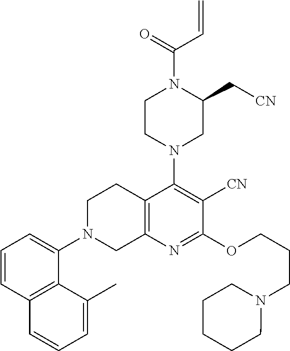 [SMILES: C=CC(=O)N1CCN(c2c(C#N)c(OCCCN3CCCCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- (piperidin-1-yl)propoxy)-5,6,7,8- tetrahydro-1,7-naphthyridine-3- carbonitrile; | 618 |
| 298 | 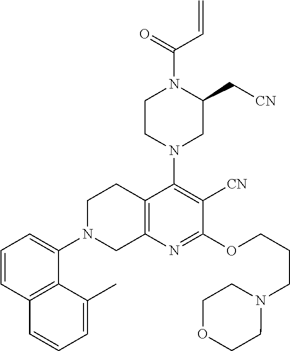 [SMILES: C=CC(=O)N1CCN(c2c(C#N)c(OCCCN3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- morpholinopropoxy)-5,6,7,8- tetrahydro-1,7-naphthyridine-3- carbonitrile; | 620 |
| 299 | 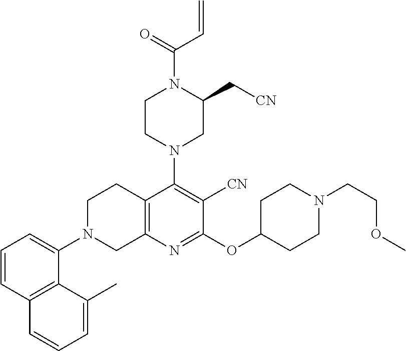 [SMILES: C=CC(=O)N1CCN(c2c(C#N)c(OC3CCN(CCOC)CC3)nc3c2CCN(c2cccc4cccc(C)c24)C3)C[C@@H]1CC#N] | (S)-4-(4-acryloyl-3- (cyanomethyl)piperazin-1-yl)-2-((1- (2-methoxyethyl)piperidin-4-yl)oxy)- 7-(8-methylnaphthalen-1-yl)-5,6,7,8- tetrahydro-1,7-naphthyridine-3- carbonitrile; | 634 |
| 300 | 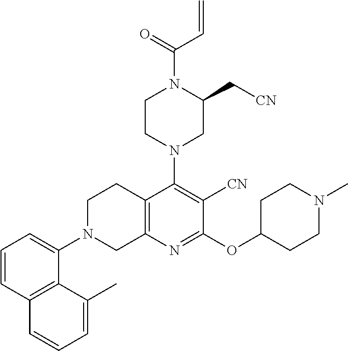 [SMILES: C=CC(=O)N1CCN(c2c(C#N)c(OC3CCN(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- methylpiperidin-4-yl)oxy)-5,6,7,8- tetrahydro-1,7-naphthyridine-3- carbonitrile; | 590 |
| 301 | 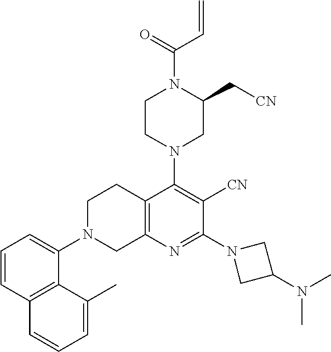 [SMILES: C=CC(=O)N1CCN(c2c(C#N)c(N3CC(N(C)C)C3)nc3c2CCN(c2cccc4cccc(C)c24)C3)C[C@@H]1CC#N] | (S)-4-(4-acryloyl-3- (cyanomethyl)piperazin-1-yl)-2-(3- (dimethylamino)azetidin-1-yl)-7-(8- methylnaphthalen-1-yl)-5,6,7,8- tetrahydro-1,7-naphthyridine-3- carbonitrile; | 575 |
| 302 | 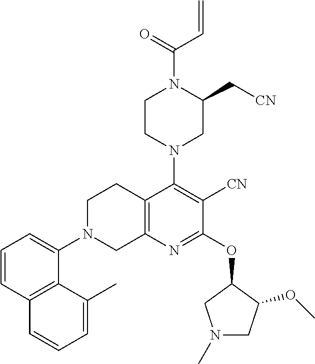 [SMILES: C=CC(=O)N1CCN(c2c(C#N)c(O[C@@H]3CN(C)C[C@H]3OC)nc3c2CCN(c2cccc4cccc(C)c24)C3)C[C@@H]1CC#N] | 4-((S)-4-acryloyl-3- (cyanomethyl)piperazin-1-yl)-2- (((3R,4R)-4-methoxy-1- methylpyrrolidin-3-yl)oxy)-7-(8- methylnaphthalen-1-yl)-5,6,7,8- tetrahydro-1,7-naphthyridine-3- carbonitrile; | 606 |
| 303 | 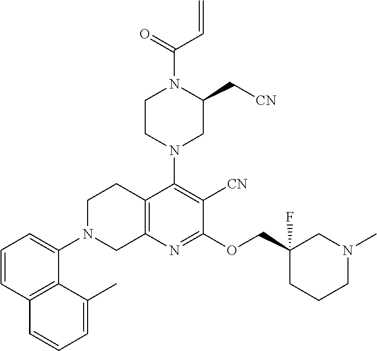 [SMILES: C=CC(=O)N1CCN(c2c(C#N)c(OC[C@]3(F)CCCN(C)C3)nc3c2CCN(c2cccc4cccc(C)c24)C3)C[C@@H]1CC#N] | 4-((S)-4-acryloyl-3- (cyanomethyl)piperazin-1-yl)-2-(((S)- 3-fluoro-1-methylpiperidin-3- yl)methoxy)-7-(8-methylnaphthalen- 1-yl)-5,6,7,8-tetrahydro-1,7- naphthyridine-3-carbonitrile; | 622 |
| 304 | 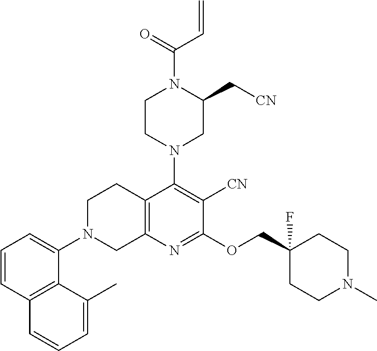 | (S)-4-(4-acryloyl-3- (cyanomethyl)piperazin-1-yl)-2-((4- fluoro-1-methylpiperidin-4- yl)methoxy)-7-(8-methylnaphthalen- 1-yl)-5,6,7,8-tetrahydro-1,7- naphthyridine-3-carbonitrile; | 622 |
| 305 | 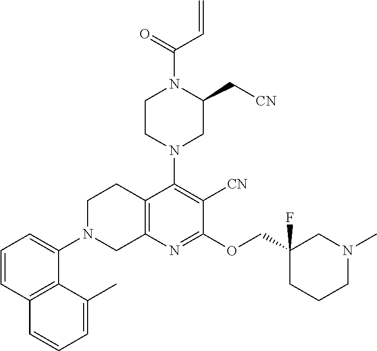 [SMILES: C=CC(=O)N1CCN(c2c(C#N)c(OC[C@@]3(F)CCCN(C)C3)nc3c2CCN(c2cccc4cccc(C)c24)C3)C[C@@H]1CC#N] | 4-((S)-4-acryloyl-3- (cyanomethyl)piperazin-1-yl)-2-(((R)- 3-fluoro-1-methylpiperidin-3- yl)methoxy)-7-(8-methylnaphthalen- 1-yl)-5,6,7,8-tetrahydro-1,7- naphthyridine-3-carbonitrile; | 622 |
| 306 | 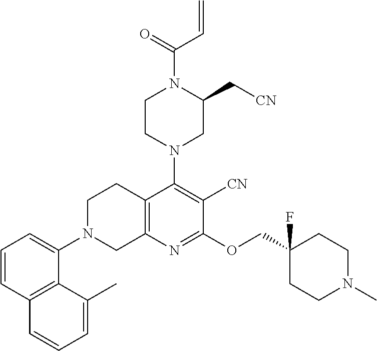 | (S)-4-(4-acryloyl-3- (cyanomethyl)piperazin-1-yl)-2-((4- fluoro-1-methylpiperidin-4- yl)methoxy)-7-(8-methylnaphthalen- 1-yl)-5,6,7,8-tetrahydro-1,7- naphthyridine-3-carbonitrile; | 622 |
| 307 | 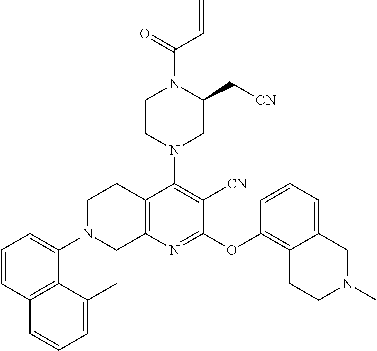 [SMILES: C=CC(=O)N1CCN(c2c(C#N)c(Oc3cccc4c3CCN(C)C4)nc3c2CCN(c2cccc4cccc(C)c24)C3)C[C@@H]1CC#N] | (S)-4-(4-acryloyl-3- (cyanomethyl)piperazin-1-yl)-2-((2- methyl-1,2,3,4-tetrahydroisoquinolin- 5-yl)oxy)-7-(8-methylnaphthalen-1- yl)-5,6,7,8-tetrahydro-1,7- naphthyridine-3-carbonitrile; | 638 |
| 308 | 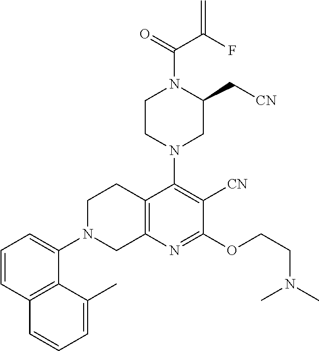 [SMILES: C=C(F)C(=O)N1CCN(c2c(C#N)c(OCCN(C)C)nc3c2CCN(c2cccc4cccc(C)c24)C3)C[C@@H]1CC#N] | (S)-4-(3-(cyanomethyl)-4-(2- fluoroacryloyl)piperazin-1-yl)-2-(2- (dimethylamino)ethoxy)-7-(8- methylnaphthalen-1-yl)-5,6,7,8- tetrahydro-1,7-naphthyridine-3- carbonitrile; | 582 |
| 309 | 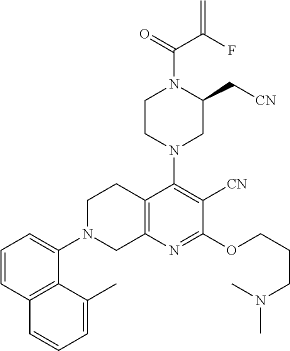 [SMILES: C=C(F)C(=O)N1CCN(c2c(C#N)c(OCCCN(C)C)nc3c2CCN(c2cccc4cccc(C)c24)C3)C[C@@H]1CC#N] | (S)-4-(3-(cyanomethyl)-4-(2- fluoroacryloyl)piperazin-1-yl)-2-(3- (dimethylamino)propoxy)-7-(8- methylnaphthalen-1-yl)-5,6,7,8- tetrahydro-1,7-naphthyridine-3- carbonitrile; | 596 |
| 310 | 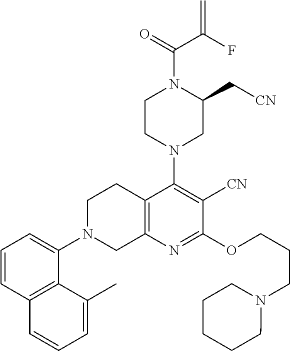 [SMILES: C=C(F)C(=O)N1CCN(c2c(C#N)c(OCCCN3CCCCC3)nc3c2CCN(c2cccc4cccc(C)c24)C3)C[C@@H]1CC#N] | (S)-4-(3-(cyanomethyl)-4-(2- fluoroacryloyl)piperazin-1-yl)-7-(8- methylnaphthalen-1-yl)-2-(3- (piperidin-1-yl)propoxy)-5,6,7,8- tetrahydro-1,7-naphthyridine-3- carbonitrile; | 636 |
| 311 | 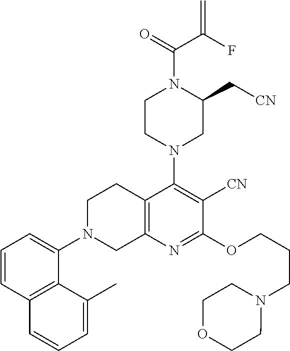 [SMILES: C=C(F)C(=O)N1CCN(c2c(C#N)c(OCCCN3CCOCC3)nc3c2CCN(c2cccc4cccc(C)c24)C3)C[C@@H]1CC#N] | (S)-4-(3-(cyanomethyl)-4-(2- fluoroacryloyl)piperazin-1-yl)-7-(8- methylnaphthalen-1-yl)-2-(3- morpholinopropoxy)-5,6,7,8- tetrahydro-1,7-naphthyridine-3- carbonitrile; | 638 |
| 312 | 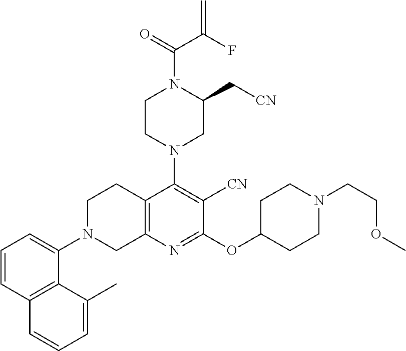 [SMILES: C=C(F)C(=O)N1CCN(c2c(C#N)c(OC3CCN(CCOC)CC3)nc3c2CCN(c2cccc4cccc(C)c24)C3)C[C@@H]1CC#N] | (S)-4-(3-(cyanomethyl)-4-(2- fluoroacryloyl)piperazin-1-yl)-2-((1- (2-methoxyethyl)piperidin-4-yl)oxy)- 7-(8-methylnaphthalen-1-yl)-5,6,7,8- tetrahydro-1,7-naphthyridine-3- carbonitrile; | 652 |
| 313 | 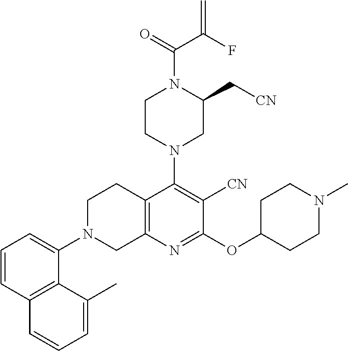 [SMILES: C=C(F)C(=O)N1CCN(c2c(C#N)c(OC3CCN(C)CC3)nc3c2CCN(c2cccc4cccc(C)c24)C3)C[C@@H]1CC#N] | (S)-4-(3-(cyanomethyl)-4-(2- fluoroacryloyl)piperazin-1-yl)-7-(8- methylnaphthalen-1-yl)-2-((1- methylpiperidin-4-yl)oxy)-5,6,7,8- tetrahydro-1,7-naphthyridine-3- carbonitrile; | 608 |
| 314 | 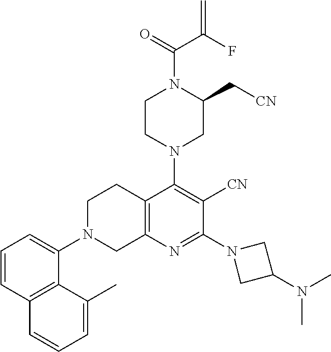 [SMILES: C=C(F)C(=O)N1CCN(c2c(C#N)c(N3CC(N(C)C)C3)nc3c2CCN(c2cccc4cccc(C)c24)C3)C[C@@H]1CC#N] | (S)-4-(3-(cyanomethyl)-4-(2- fluoroacryloyl)piperazin-1-yl)-2-(3- (dimethylamino)azetidin-1-yl)-7-(8- methylnaphthalen-1-yl)-5,6,7,8- tetrahydro-1,7-naphthyridine-3- carbonitrile; | 593 |
| 315 | 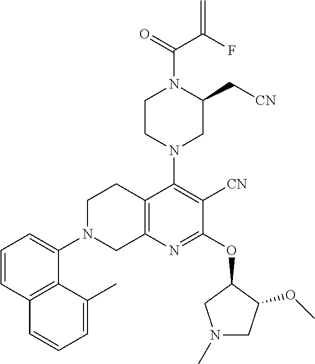 [SMILES: C=C(F)C(=O)N1CCN(c2c(C#N)c(O[C@@H]3CN(C)C[C@H]3OC)nc3c2CCN(c2cccc4cccc(C)c24)C3)C[C@@H]1CC#N] | 4-((S)-3-(cyanomethyl)-4-(2- fluoroacryloyl)piperazin-1-yl)-2- (((3R,4R)-4-methoxy-1- methylpyrrolidin-3-yl)oxy)-7-(8- methylnaphthalen-1-yl)-5,6,7,8- tetrahydro-1,7-naphthyridine-3- carbonitrile; | 624 |
| 316 | 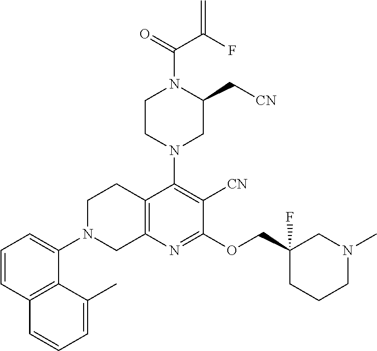 [SMILES: C=C(F)C(=O)N1CCN(c2c(C#N)c(OC[C@]3(F)CCCN(C)C3)nc3c2CCN(c2cccc4cccc(C)c24)C3)C[C@@H]1CC#N] | 4-((S)-3-(cyanomethyl)-4-(2- fluoroacryloyl)piperazin-1-yl)-2-(((S)- 3-fluoro-1-methylpiperidin-3- yl)methoxy)-7-(8-methylnaphthalen- 1-yl)-5,6,7,8-tetrahydro-1,7- naphthyridine-3-carbonitrile | 640 |
| 317 | 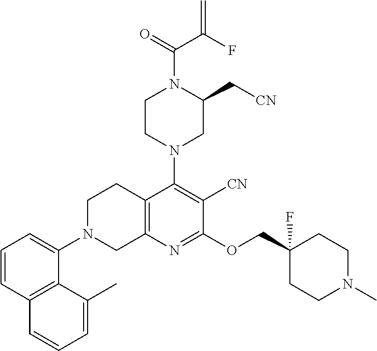 | (S)-4-(3-(cyanomethyl)-4-(2- fluoroacryloyl)piperazin-1-yl)-2-((4- fluoro-1-methylpiperidin-4- yl)methoxy)-7-(8-methylnaphthalen- 1-yl)-5,6,7,8-tetrahydro-1,7- naphthyridine-3-carbonitrile; | 640 |
| 318 | 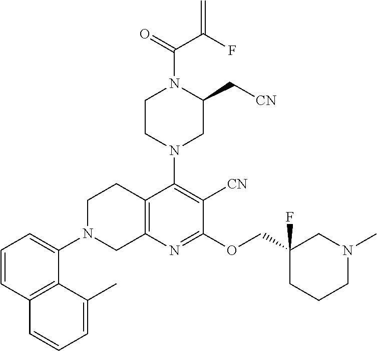 [SMILES: C=C(F)C(=O)N1CCN(c2c(C#N)c(OC[C@@]3(F)CCCN(C)C3)nc3c2CCN(c2cccc4cccc(C)c24)C3)C[C@@H]1CC#N] | 4-((S)-3-(cyanomethyl)-4-(2- fluoroacryloyl)piperazin-1-yl)-2- (((R)-3-fluoro-1-methylpiperidin-3- yl)methoxy)-7-(8-methylnaphthalen- 1-yl)-5,6,7,8-tetrahydro-1,7- naphthyridine-3-carbonitrile; | 640 |
| 319 | 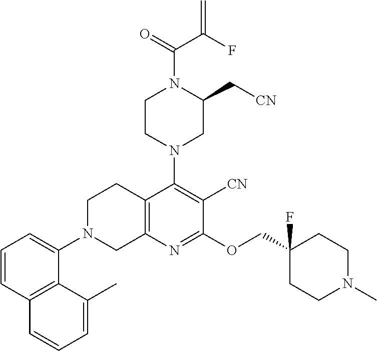 | (S)-4-(3-(cyanomethyl)-4-(2- fluoroacryloyl)piperazin-1-yl)-2-((4- fluoro-1-methylpiperidin-4- yl)methoxy)-7-(8-methylnaphthalen- 1-yl)-5,6,7,8-tetrahydro-1,7- naphthyridine-3-carbonitrile; | 640 |
| 320 | 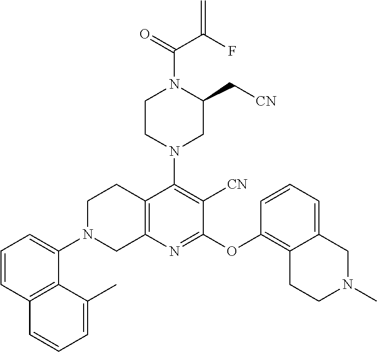 [SMILES: C=C(F)C(=O)N1CCN(c2c(C#N)c(Oc3cccc4c3CCN(C)C4)nc3c2CCN(c2cccc4cccc(C)c24)C3)C[C@@H]1CC#N] | (S)-4-(3-(cyanomethyl)-4-(2- fluoroacryloyl)piperazin-1-yl)-2-((2- methyl-1,2,3,4-tetrahydroisoquinolin- 5-3,4-7-(8-methylnaphthalen-1-yl)- 5,6,7,8-tetrahydro-1,7-naphthyridine- 3-carbonitrile; | 656 |
| 321 | 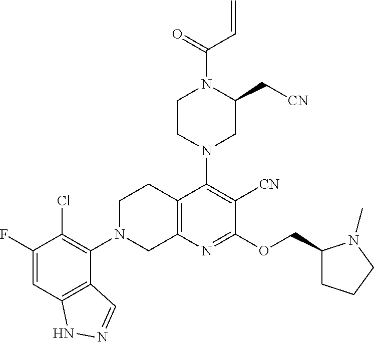 [SMILES: C=CC(=O)N1CCN(c2c(C#N)c(OC[C@@H]3CCCN3C)nc3c2CCN(c2c(Cl)c(F)cc4[nH]ncc24)C3)C[C@@H]1CC#N] | 4-((S)-4-acryloyl-3- (cyanomethyl)piperazin-1-yl)-7-(5- chloro-6-fluoro-1H-indazol-4-yl)-2- (((S)-1-methylpyrrolidin-2- yl)methoxy)-5,6,7,8-tetrahydro-1,7- naphthyridine-3-carbonitrile; | 618 |
| 322 | 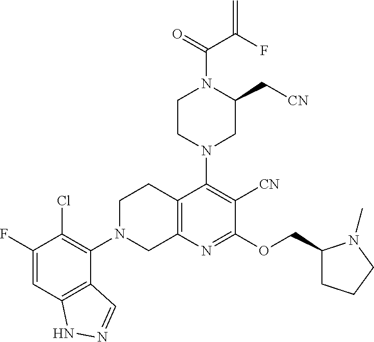 [SMILES: C=C(F)C(=O)N1CCN(c2c(C#N)c(OC[C@@H]3CCCN3C)nc3c2CCN(c2c(Cl)c(F)cc4[nH]ncc24)C3)C[C@@H]1CC#N] | 7-(5-chloro-6-fluoro-1H-indazol-4- yl)-4-((S)-3-(cyanomethyl)-4-(2- fluoroacryloyl)piperazin-1-yl)-2-(((S)- 1-methylpyrrolidin-2-yl)methoxy)- 5,6,7,8-tetrahydro-1,7-naphthyridine- 3-carbonitrile; | 636 |
| 323 | 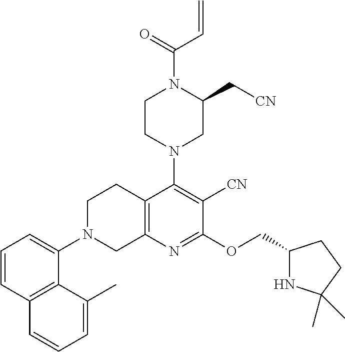 [SMILES: C=CC(=O)N1CCN(c2c(C#N)c(OC[C@@H]3CCC(C)(C)N3)nc3c2CCN(c2cccc4cccc(C)c24)C3)C[C@@H]1CC#N] | 4-((S)-4-acryloyl-3- (cyanomethyl)piperazin-1-yl)-2-(((S)- 5,5-dimethylpyrrolidin-2- yl)methoxy)-7-(8-methylnaphthalen- 1-yl)-5,6,7,8-tetrahydro-1,7- naphthyridine-3-carbonitrile; | 604 |
| 324 | 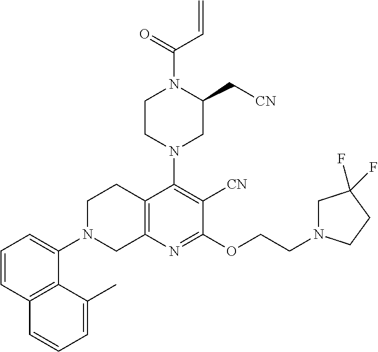 [SMILES: C=CC(=O)N1CCN(c2c(C#N)c(OCCN3CCC(F)(F)C3)nc3c2CCN(c2cccc4cccc(C)c24)C3)C[C@@H]1CC#N] | (S)-4-(4-acryloyl-3- (cyanomethyl)piperazin-1-yl)-2-(2- (3,3-difluoropyrrolidin-1-yl)ethoxy)- 7-(8-methylnaphthalen-1-yl)-5,6,7,8- tetrahydro-1,7-naphthyridine-3- carbonitrile; | 626 |
| 325 | 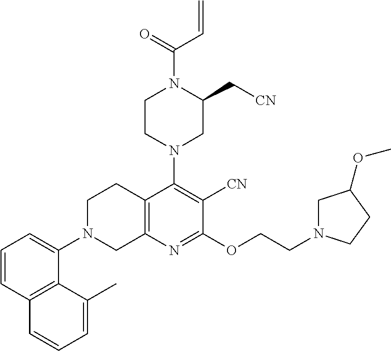 [SMILES: C=CC(=O)N1CCN(c2c(C#N)c(OCCN3CCC(OC)C3)nc3c2CCN(c2cccc4cccc(C)c24)C3)C[C@@H]1CC#N] | 4-((S)-4-acryloyl-3- (cyanomethyl)piperazin-1-yl)-2-(2-(3- methoxypyrrolidin-1-yl)ethoxy)-7-(8- methylnaphthalen-1-yl)-5,6,7,8- tetrahydro-1,7-naphthyridine-3- carbonitrile; | 620 |
| 326 | 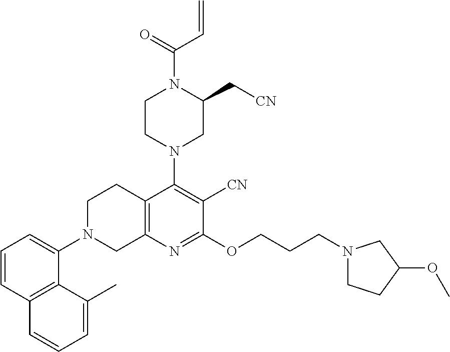 [SMILES: C=CC(=O)N1CCN(c2c(C#N)c(OCCCN3CCC(OC)C3)nc3c2CCN(c2cccc4cccc(C)c24)C3)C[C@@H]1CC#N] | 4-((S)-4-acryloyl-3- (cyanomethyl)piperazin-1-yl)-2-(3-(3- methoxypyrrolidin-1-yl)propoxy)-7- (8-methylnaphthalen-1-yl)-5,6,7,8- tetrahydro-1,7-naphthyridine-3- carbonitrile; | 634 |
| 327 | 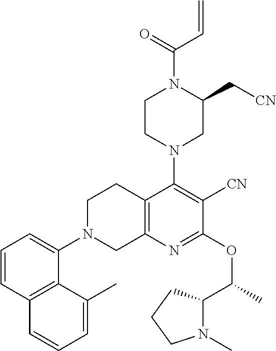 [SMILES: C=CC(=O)N1CCN(c2c(C#N)c(O[C@H](C)[C@H]3CCCN3C)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((R)-1-((R)- 1-methylpyrrolidin-2-yl)ethoxy)- 5,6,7,8-tetrahydro-1,7-naphthyridine- 3-carbonitrile; | 604 |
| 328 | 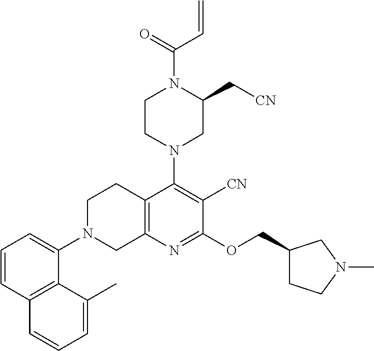 [SMILES: C=CC(=O)N1CCN(c2c(C#N)c(OC[C@@H]3CCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((R)-1- methylpyrrolidin-3-yl)methoxy)- 5,6,7,8-tetrahydro-1,7-naphthyridine- 3-carbonitrile; | 590 |
| 329 | 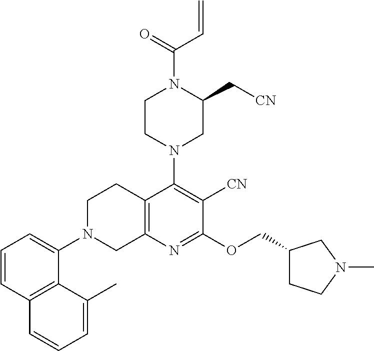 [SMILES: C=CC(=O)N1CCN(c2c(C#N)c(OC[C@H]3CCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((S)-1- methylpyrrolidin-3-yl)methoxy)- 5,6,7,8-tetrahydro-1,7-naphthyridine- 3-carbonitrile; | 590 |
| 330 | 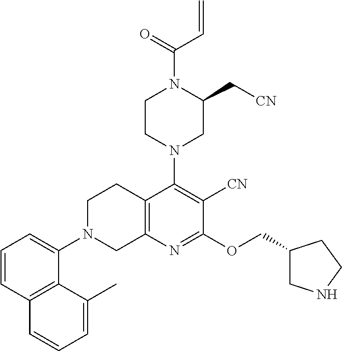 [SMILES: C=CC(=O)N1CCN(c2c(C#N)c(OC[C@@H]3CCNC3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((R)- pyrrolidin-3-yl)methoxy)-5,6,7,8- tetrahydro-1,7-naphthyridine-3- carbonitrile; | 576 |
| 331 | 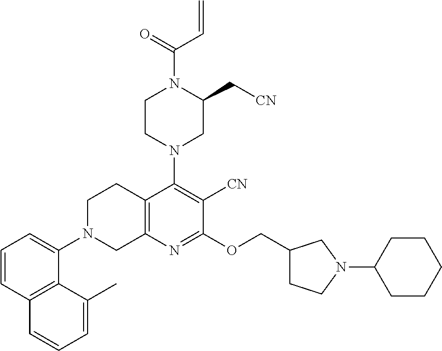 [SMILES: C=CC(=O)N1CCN(c2c(C#N)c(OCC3CCN(C4CCCCC4)C3)nc3c2CCN(c2cccc4cccc(C)c24)C3)C[C@@H]1CC#N] | 4-((S)-4-acryloyl-3- (cyanomethyl)piperazin-1-yl)-2-((1- cyclohexylpyrrolidin-3-yl)methoxy)- 7-(8-methylnaphthalen-1-yl)-5,6,7,8- tetrahydro-1,7-naphthyridine-3- carbonitrile; | 658 |
| 332 | 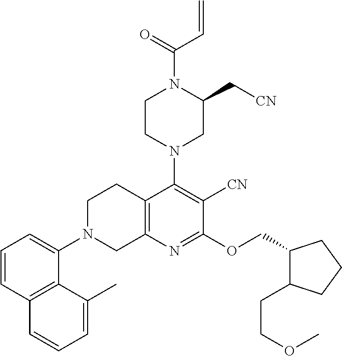 [SMILES: C=CC(=O)N1CCN(c2c(C#N)c(OC[C@@H]3CCCC3CCOC)nc3c2CCN(c2cccc4cccc(C)c24)C3)C[C@@H]1CC#N] | 4-((S)-4-acryloyl-3- (cyanomethyl)piperazin-1-yl)-2-(((S)- 1-(2-methoxyethyl)pyrrolidin-2- yl)methoxy)-7-(8-methylnaphthalen- 1-yl)-5,6,7,8-tetrahydro-1,7- naphthyridine-3-carbonitrile; | 634 |
| 333 | 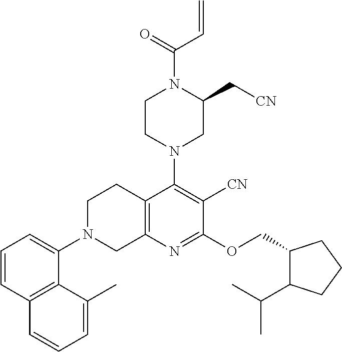 [SMILES: C=CC(=O)N1CCN(c2c(C#N)c(OC[C@@H]3CCCC3C(C)C)nc3c2CCN(c2cccc4cccc(C)c24)C3)C[C@@H]1CC#N] | 4-((S)-4-acryloyl-3- (cyanomethyl)piperazin-1-yl)-2-(((S)- 1-isopropylpyrrolidin-2-yl)methoxy)- 7-(8-methylnaphthalen-1-yl)-5,6,7,8- tetrahydro-1,7-naphthyridine-3- carbonitrile; | 618 |
| 334 | 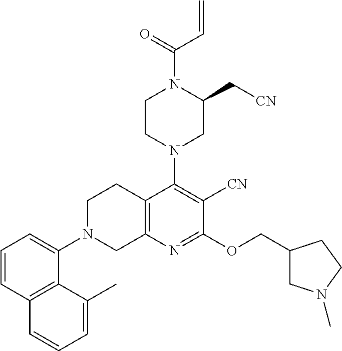 [SMILES: C=CC(=O)N1CCN(c2c(C#N)c(OCC3CCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((1- methylpyrrolidin-3-yl)methoxy)- 5,6,7,8-tetrahydro-1,7-naphthyridine- 3-carbonitrile; | 590 |
| 335 | 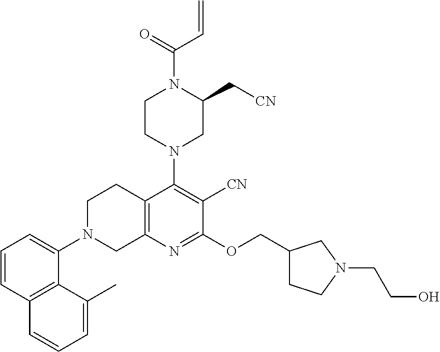 [SMILES: C=CC(=O)N1CCN(c2c(C#N)c(OCC3CCN(CCO)C3)nc3c2CCN(c2cccc4cccc(C)c24)C3)C[C@@H]1CC#N] | 4-((S)-4-acryloyl-3- (cyanomethyl)piperazin-1-yl)-2-((1- (2-hydroxyethyl)pyrrolidin-3- yl)methoxy)-7-(8-methylnaphthalen- 1-yl)-5,6,7,8-tetrahydro-1,7- naphthyridine-3-carbonitrile; | 620 |
| 336 | 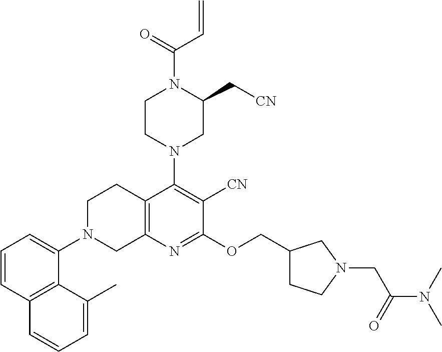 [SMILES: C=CC(=O)N1CCN(c2c(C#N)c(OCC3CCN(CC(=O)N(C)C)C3)nc3c2CCN(c2cccc4cccc(C)c24)C3)C[C@@H]1CC#N] | 2-(3-(((4-((S)-4-acryloyl-3- (cyanomethyl)piperazin-1-yl)-3- cyano-7-(8-methylnaphthalen-1-yl)- 5,6,7,8-tetrahydro-1,7-naphthyridin-2- yl)oxy)methyl)pyrrolidin-1-yl)-N,N- dimethylacetamide; | 661 |
| 337 | 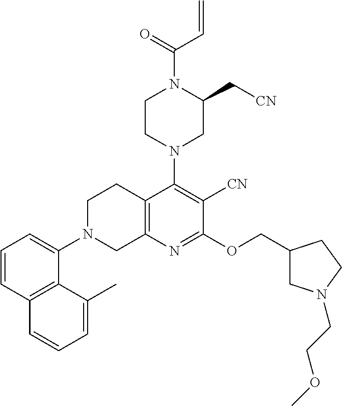 [SMILES: C=CC(=O)N1CCN(c2c(C#N)c(OCC3CCN(CCOC)C3)nc3c2CCN(c2cccc4cccc(C)c24)C3)C[C@@H]1CC#N] | 4-((S)-4-acryloyl-3- (cyanomethyl)piperazin-1-yl)-2-((1- (2-methoxyethyl)pyrrolidin-3- yl)methoxy)-7-(8-methylnaphthalen- 1-yl)-5,6,7,8-tetrahydro-1,7- naphthyridine-3-carbonitrile; | 634 |
| 338 | 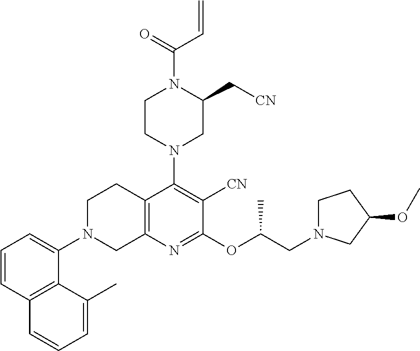 [SMILES: C=CC(=O)N1CCN(c2c(C#N)c(O[C@H](C)CN3CC[C@@H](OC)C3)nc3c2CCN(c2cccc4cccc(C)c24)C3)C[C@@H]1CC#N] | 4-((S)-4-acryloyl-3- (cyanomethyl)piperazin-1-yl)-2-(((R)- 1-((R)-3-methoxypyrrolidin-1- yl)propan-2-yl)oxy)-7-(8- methylnaphthalen-1-yl)-5,6,7,8- tetrahydro-1,7-naphthyridine-3- carbonitrile; | 634 |
| 339 | 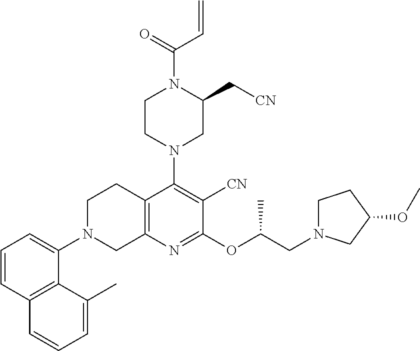 [SMILES: C=CC(=O)N1CCN(c2c(C#N)c(O[C@H](C)CN3CC[C@H](OC)C3)nc3c2CCN(c2cccc4cccc(C)c24)C3)C[C@@H]1CC#N] | 4-((S)-4-acryloyl-3- (cyanomethyl)piperazin-1-yl)-2-(((R)- 1-((S)-3-methoxypyrrolidin-1- yl)propan-2-yl)oxy)-7-(8-methyl-4 a,8a-dihydronaphthalen-1-yl)-5,6,7,8- tetrahydro-1,7-naphthyridine-3- carbonitrile; | 636 |
| 340 | 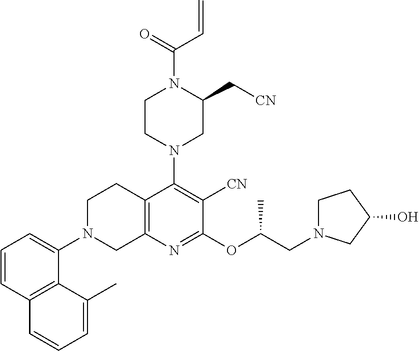 [SMILES: C=CC(=O)N1CCN(c2c(C#N)c(O[C@H](C)CN3CC[C@H](O)C3)nc3c2CCN(c2cccc4cccc(C)c24)C3)C[C@@H]1CC#N] | 4-((S)-4-acryloyl-3- (cyanomethyl)piperazin-1-yl)-2-(((R)- 1-((S)-3-hydroxypyrrolidin-1- yl)propan-2-yl)oxy)-7-(8- methylnaphthalen-1-yl)-5,6,7,8- tetrahydro-1,7-naphthyridine-3- carbonitrile; | 620 |
| 341 | 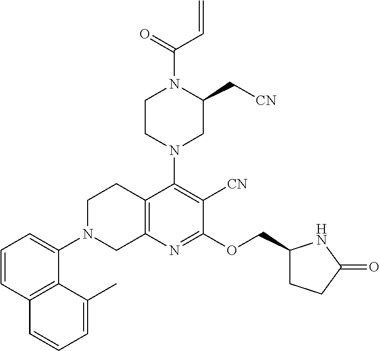 [SMILES: C=CC(=O)N1CCN(c2c(C#N)c(OC[C@@H]3CCC(=O)N3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((S)-5- oxopyrrolidin-2-yl)methoxy)-5,6,7,8- tetrahydro-1,7-naphthyridine-3- carbonitrile; | 590 |
| 342 | 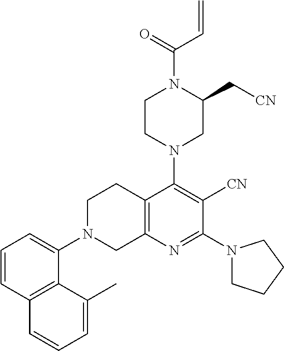 [SMILES: C=CC(=O)N1CCN(c2c(C#N)c(N3CCCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(pyrrolidin- 1-yl)-5,6,7,8-tetrahydro-1,7- naphthyridine-3-carbonitrile; | 546 |
| 343 | 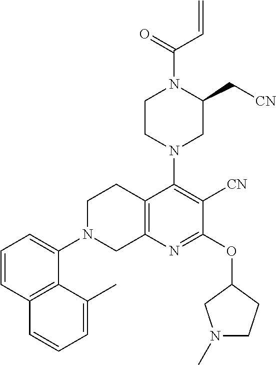 [SMILES: C=CC(=O)N1CCN(c2c(C#N)c(OC3CCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((1- methylpyrrolidin-3-yl)oxy)-5,6,7,8- tetrahydro-1,7-naphthyridine-3- carbonitrile; | 576 |
| 344 | 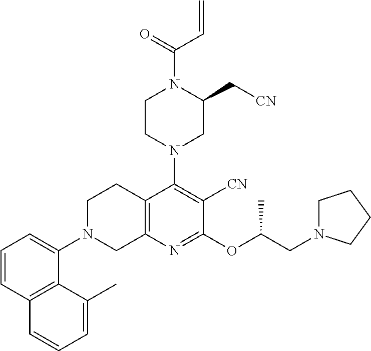 [SMILES: C=CC(=O)N1CCN(c2c(C#N)c(O[C@H](C)CN3CCCC3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((R)-1- (pyrrolidin-1-yl)propan-2-yl)oxy)- 5,6,7,8-tetrahydro-1,7-naphthyridine- 3-carbonitrile; | 604 |
| 345 | 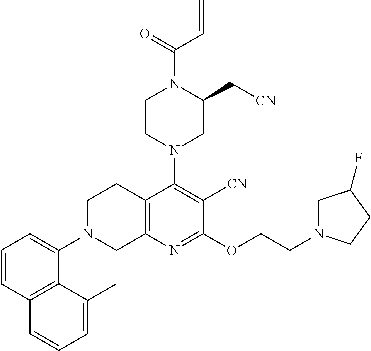 [SMILES: C=CC(=O)N1CCN(c2c(C#N)c(OCCN3CCC(F)C3)nc3c2CCN(c2cccc4cccc(C)c24)C3)C[C@@H]1CC#N] | 4-((S)-4-acryloyl-3- (cyanomethyl)piperazin-1-yl)-2-(2-(3- fluoropyrrolidin-1-yl)ethoxy)-7-(8- methylnaphthalen-1-yl)-5,6,7,8- tetrahydro-1,7-naphthyridine-3- carbonitrile; | 608 |
| 346 | 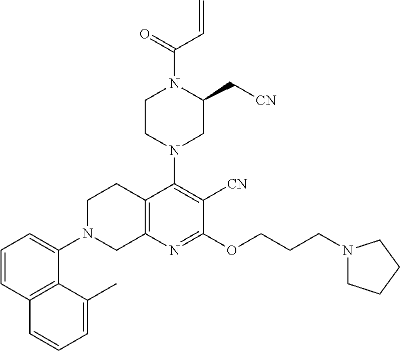 [SMILES: C=CC(=O)N1CCN(c2c(C#N)c(OCCCN3CCCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- (pyrrolidin-1-yl)propoxy)-5,6,7,8- tetrahydro-1,7-naphthyridine-3- carbonitrile; | 604 |
| 347 | 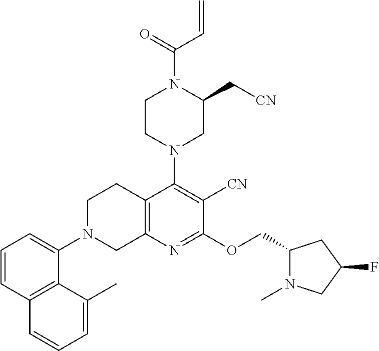 [SMILES: C=CC(=O)N1CCN(c2c(C#N)c(OC[C@@H]3C[C@@H](F)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4R)-4-fluoro-1- methylpyrrolidin-2-yl)methoxy)-7-(8- methylnaphthalen-1-yl)-5,6,7,8- tetrahydro-1,7-naphthyridine-3- carbonitrile; | 608 |
| 348 | 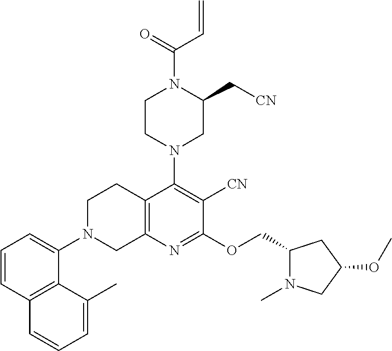 [SMILES: C=CC(=O)N1CCN(c2c(C#N)c(OC[C@@H]3C[C@H](OC)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4S)-4-methoxy-1- methylpyrrolidin-2-yl)methoxy)-7-(8- methylnaphthalen-1-yl)-5,6,7,8- tetrahydro-1,7-naphthyridine-3- carbonitrile; | 620 |
| 349 | 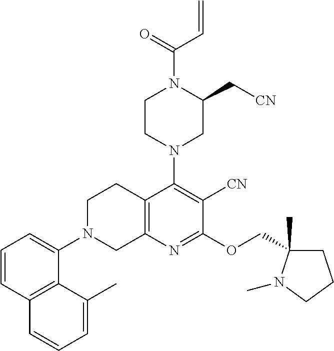 [SMILES: C=CC(=O)N1CCN(c2c(C#N)c(OC[C@]3(C)CCCN3C)nc3c2CCN(c2cccc4cccc(C)c24)C3)C[C@@H]1CC#N] | 4-((S)-4-acryloyl-3- (cyanomethyl)piperazin-1-yl)-2-(((S)- 1,2-dimethylpyrrolidin-2- yl)methoxy)-7-(8-methylnaphthalen- 1-yl)-5,6,7,8-tetrahydro-1,7- naphthyridine-3-carbonitrile; | 604 |
| 350 | 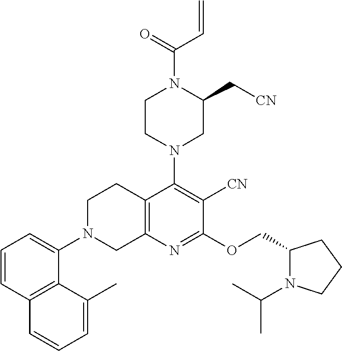 [SMILES: C=CC(=O)N1CCN(c2c(C#N)c(OC[C@@H]3CCCN3C(C)C)nc3c2CCN(c2cccc4cccc(C)c24)C3)C[C@@H]1CC#N] | 4-((S)-4-acryloyl-3- (cyanomethyl)piperazin-1-yl)-2-(((S)- 1-isopropylpyrrolidin-2-yl)methoxy)- 7-(8-methylnaphthalen-1-yl)-5,6,7,8- tetrahydro-1,7-naphthyridine-3- carbonitrile; | 618 |
| 351 | 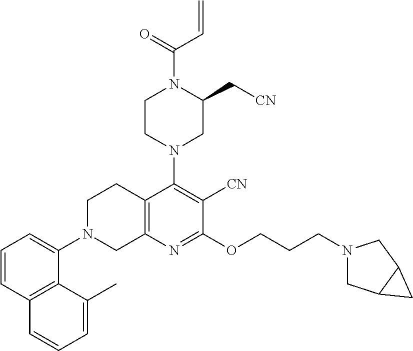 [SMILES: C=CC(=O)N1CCN(c2c(C#N)c(OCCCN3CC4CC4C3)nc3c2CCN(c2cccc4cccc(C)c24)C3)C[C@@H]1CC#N] | 2-(3-(3-azabicyclo[3.1.0]hexan-3- yl)propoxy)-4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 616 |
| 352 | 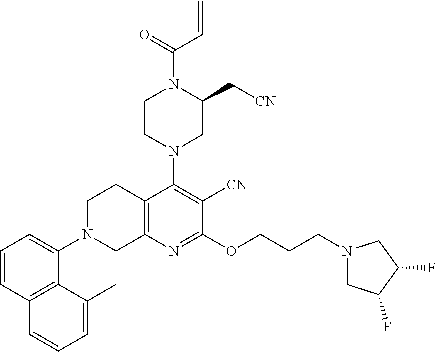 [SMILES: C=CC(=O)N1CCN(c2c(C#N)c(OCCCN3C[C@@H](F)[C@@H](F)C3)nc3c2CCN(c2cccc4cccc(C)c24)C3)C[C@@H]1CC#N] | 4-((S)-4-acryloyl-3- (cyanomethyl)piperazin-1-yl)-2-(3- ((3R,4S)-3,4-difluoropyrrolidin-1- yl)propoxy)-7-(8-methylnaphthalen-1- yl)-5,6,7,8-tetrahydro-1,7- naphthyridine-3-carbonitrile; | 640 |
| 353 | 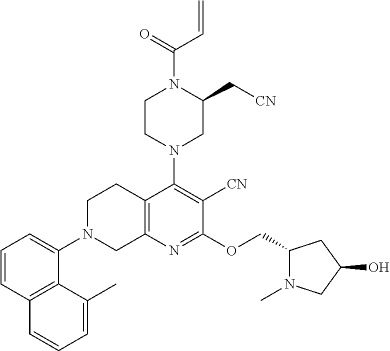 [SMILES: C=CC(=O)N1CCN(c2c(C#N)c(OC[C@@H]3C[C@@H](O)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4R)-4-hydroxy-1- methylpyrrolidin-2-yl)methoxy)-7-(8- methylnaphthalen-1-yl)-5,6,7,8- tetrahydro-1,7-naphthyridine-3- carbonitrile; | 606 |
| 354 | 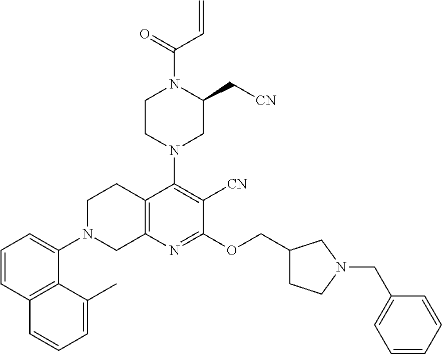 [SMILES: C=CC(=O)N1CCN(c2c(C#N)c(OCC3CCN(Cc4ccccc4)C3)nc3c2CCN(c2cccc4cccc(C)c24)C3)C[C@@H]1CC#N] | 4-((S)-4-acryloyl-3- (cyanomethyl)piperazin-1-yl)-2-((1- benzylpyrrolidin-3-yl)methoxy)-7-(8- methylnaphthalen-1-yl)-5,6,7,8- tetrahydro-1,7-naphthyridine-3- carbonitrile; | 666 |
| 355 | 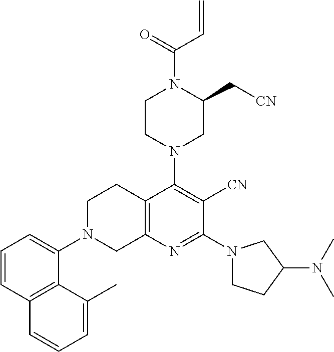 [SMILES: C=CC(=O)N1CCN(c2c(C#N)c(N3CCC(N(C)C)C3)nc3c2CCN(c2cccc4cccc(C)c24)C3)C[C@@H]1CC#N] | 4-((S)-4-acryloyl-3- (cyanomethyl)piperazin-1-yl)-2-(3- (dimethylamino)pyrrolidin-1-yl)-7-(8- methylnaphthalen-1-yl)-5,6,7,8- tetrahydro-1,7-naphthyridine-3- carbonitrile; | 589 |
| 356 | 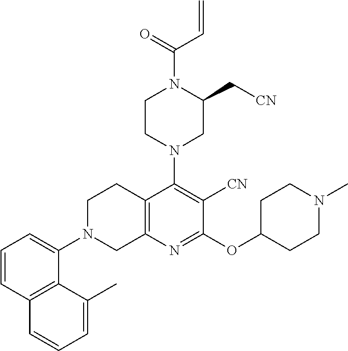 [SMILES: C=CC(=O)N1CCN(c2c(C#N)c(OC3CCN(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- methylpiperidin-4-yl)oxy)-5,6,7,8- tetrahydro-1,7-naphthyridine-3- carbonitrile; | 590 |
| 357 | 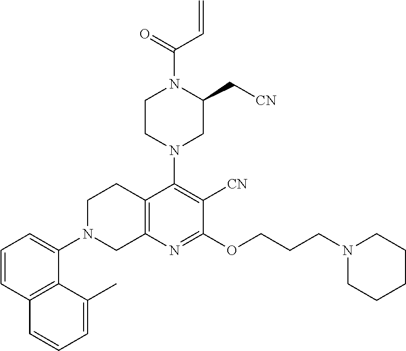 [SMILES: C=CC(=O)N1CCN(c2c(C#N)c(OCCCN3CCCCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- (piperidin-1-yl)propoxy)-5,6,7,8- tetrahydro-1,7-naphthyridine-3- carbonitrile; | 618 |
| 358 | 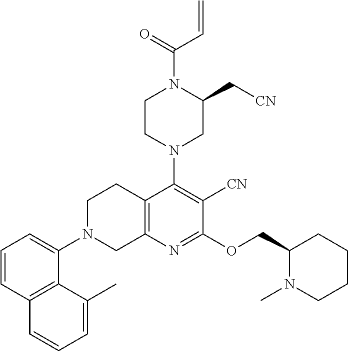 [SMILES: C=CC(=O)N1CCN(c2c(C#N)c(OC[C@H]3CCCCN3C)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((R)-1- methylpiperidin-2-yl)methoxy)- 5,6,7,8-tetrahydro-1,7-naphthyridine- 3-carbonitrile; | 604 |
| 359 | 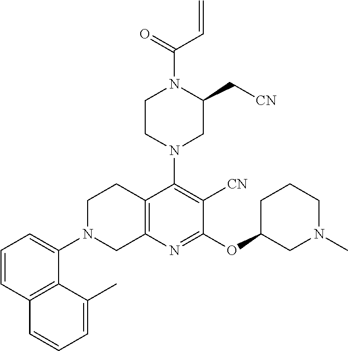 [SMILES: C=CC(=O)N1CCN(c2c(C#N)c(O[C@H]3CCCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-(((S)-1- methylpiperidin-3-yl)oxy)-5,6,7,8- tetrahydro-1,7-naphthyridine-3- carbonitrile; | 590 |
| 360 | 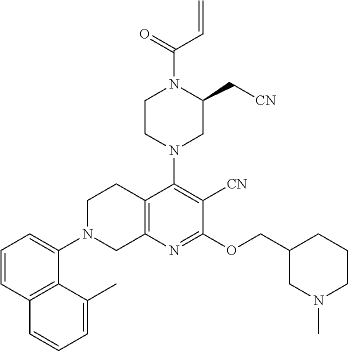 [SMILES: C=CC(=O)N1CCN(c2c(C#N)c(OCC3CCCN(C)C3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((1- methylpiperidin-3-yl)methoxy)- 5,6,7,8-tetrahydro-1,7-naphthyridine- 3-carbonitrile; | 604 |
| 361 | 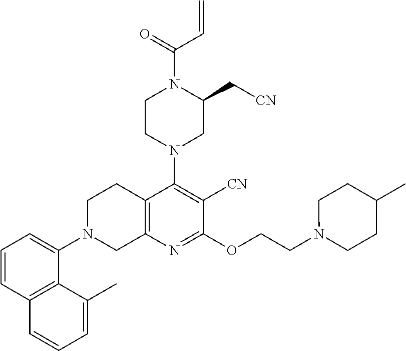 [SMILES: C=CC(=O)N1CCN(c2c(C#N)c(OCCN3CCC(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2-(4- methylpiperidin-1-yl)ethoxy)-5,6,7,8- tetrahydro-1,7-naphthyridine-3- carbonitrile; | 618 |
| 362 | 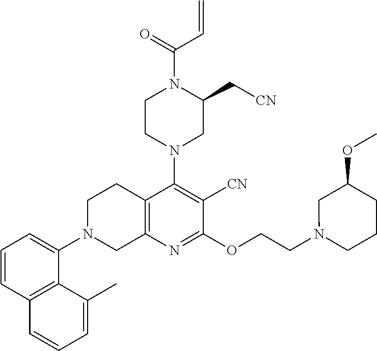 [SMILES: C=CC(=O)N1CCN(c2c(C#N)c(OCCN3CCC[C@H](OC)C3)nc3c2CCN(c2cccc4cccc(C)c24)C3)C[C@@H]1CC#N] | 4-((S)-4-acryloyl-3- (cyanomethyl)piperazin-1-yl)-2-(2- ((S)-3-methoxypiperidin-1-yl)ethoxy)- 7-(8-methylnaphthalen-1-yl)-5,6,7,8- tetrahydro-1,7-naphthyridine-3- carbonitrile; | 634 |
| 363 | 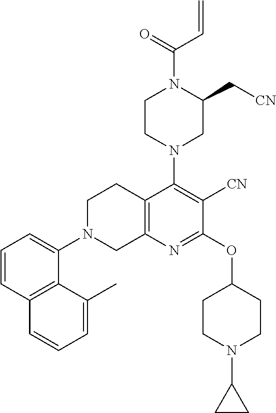 [SMILES: C=CC(=O)N1CCN(c2c(C#N)c(OC3CCN(C4CC4)CC3)nc3c2CCN(c2cccc4cccc(C)c24)C3)C[C@@H]1CC#N] | (S)-4-(4-acryloyl-3- (cyanomethyl)piperazin-1-yl)-2-((1- cyclopropylpiperidin-4-yl)oxy)-7-(8- methylnaphthalen-1-yl)-5,6,7,8- tetrahydro-1,7-naphthyridine-3- carbonitrile; | 616 |
| 364 | 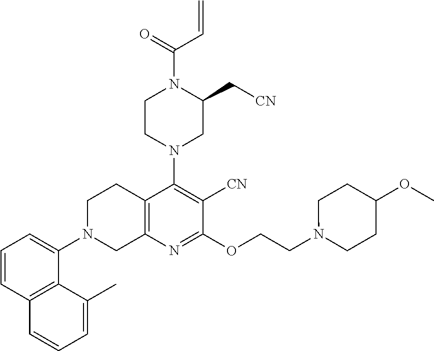 [SMILES: C=CC(=O)N1CCN(c2c(C#N)c(OCCN3CCC(OC)CC3)nc3c2CCN(c2cccc4cccc(C)c24)C3)C[C@@H]1CC#N] | (S)-4-(4-acryloyl-3- (cyanomethyl)piperazin-1-yl)-2-(2-(4- methoxypiperidin-1-yl)ethoxy)-7-(8- methylnaphthalen-1-yl)-5,6,7,8- tetrahydro-1,7-naphthyridine-3- carbonitrile; | 634 |
| 365 | 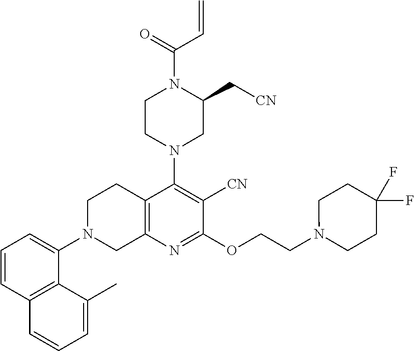 [SMILES: C=CC(=O)N1CCN(c2c(C#N)c(OCCN3CCC(F)(F)CC3)nc3c2CCN(c2cccc4cccc(C)c24)C3)C[C@@H]1CC#N] | (S)-4-(4-acryloyl-3- (cyanomethyl)piperazin-1-yl)-2-(2- (4,4-difluoropiperidin-1-yl)ethoxy)-7- (8-methylnaphthalen-1-yl)-5,6,7,8- tetrahydro-1,7-naphthyridine-3- carbonitrile; | 640 |
| 366 | 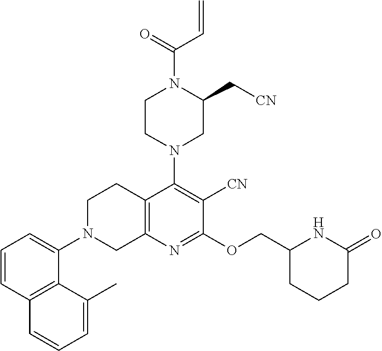 [SMILES: C=CC(=O)N1CCN(c2c(C#N)c(OCC3CCCC(=O)N3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((6- oxopiperidin-2-yl)methoxy)-5,6,7,8- tetrahydro-1,7-naphthyridine-3- carbonitrile; | 604 |
| 367 | 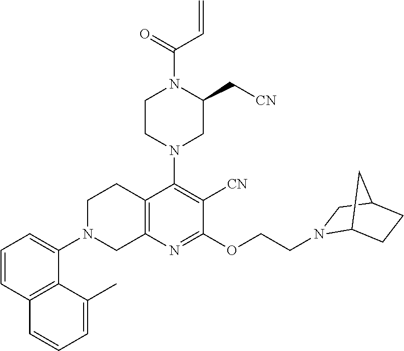 [SMILES: C=CC(=O)N1CCN(c2c(C#N)c(OCCN3CC4CCC3C4)nc3c2CCN(c2cccc4cccc(C)c24)C3)C[C@@H]1CC#N] | 2-(2-((1S,4R)-2- azabicyclo[2.2.1]heptan-2-yl)ethoxy)- 4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 616 |
| 368 | 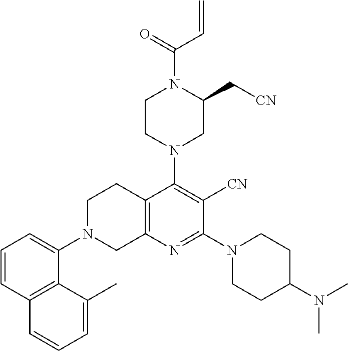 [SMILES: C=CC(=O)N1CCN(c2c(C#N)c(N3CCC(N(C)C)CC3)nc3c2CCN(c2cccc4cccc(C)c24)C3)C[C@@H]1CC#N] | (S)-4-(4-acryloyl-3- (cyanomethyl)piperazin-1-yl)-2-(4- (dimethylamino)piperidin-1-yl)-7-(8- methylnaphthalen-1-yl)-5,6,7,8- tetrahydro-1,7-naphthyridine-3- carbonitrile; | 603 |
| 369 | 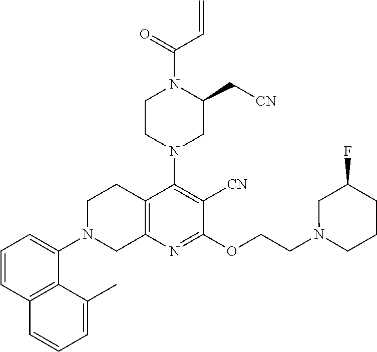 [SMILES: C=CC(=O)N1CCN(c2c(C#N)c(OCCN3CCC[C@H](F)C3)nc3c2CCN(c2cccc4cccc(C)c24)C3)C[C@@H]1CC#N] | 4-((S)-4-acryloyl-3- (cyanomethyl)piperazin-1-yl)-2-(2- ((S)-3-fluoropiperidin-1-yl)ethoxy)-7- (8-methylnaphthalen-1-yl)-5,6,7,8- tetrahydro-1,7-naphthyridine-3- carbonitrile; | 622 |
| 370 | 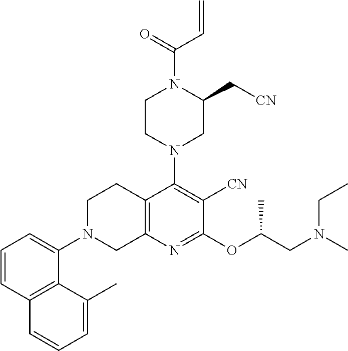 [SMILES: C=CC(=O)N1CCN(c2c(C#N)c(O[C@H](C)CN(C)CC)nc3c2CCN(c2cccc4cccc(C)c24)C3)C[C@@H]1CC#N] | 4-((S)-4-acryloyl-3- (cyanomethyl)piperazin-1-yl)-2-(((R)- 1-(ethyl(methyl)amino)propan-2- yl)oxy)-7-(8-methylnaphthalen-1-yl)- 5,6,7,8-tetrahydro-1,7-naphthyridine- 3-carbonitrile; | 592 |
| 371 | 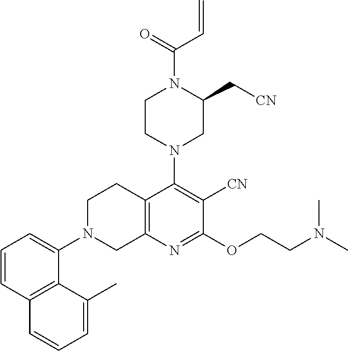 [SMILES: C=CC(=O)N1CCN(c2c(C#N)c(OCCN(C)C)nc3c2CCN(c2cccc4cccc(C)c24)C3)C[C@@H]1CC#N] | (S)-4-(4-acryloyl-3- (cyanomethyl)piperazin-1-yl)-2-(2- (dimethylamino)ethoxy)-7-(8- methylnaphthalen-1-yl)-5,6,7,8- tetrahydro-1,7-naphthyridine-3- carbonitrile; | 564 |
| 372 | 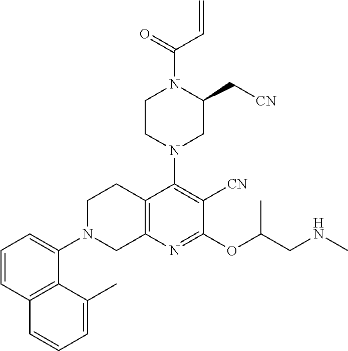 [SMILES: C=CC(=O)N1CCN(c2c(C#N)c(OC(C)CNC)nc3c2CCN(c2cccc4cccc(C)c24)C3)C[C@@H]1CC#N] | 4-((S)-4-acryloyl-3- (cyanomethyl)piperazin-1-yl)-2-((1- (methylamino)propan-2-yl)oxy)-7-(8- methylnaphthalen-1-yl)-5,6,7,8- tetrahydro-1,7-naphthyridine-3- carbonitrile; | 564 |
| 373 | 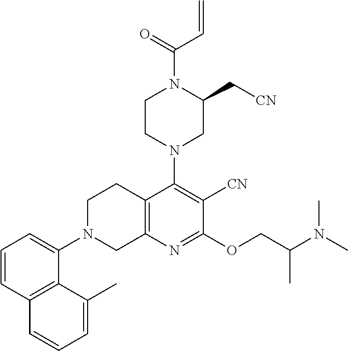 [SMILES: C=CC(=O)N1CCN(c2c(C#N)c(OCC(C)N(C)C)nc3c2CCN(c2cccc4cccc(C)c24)C3)C[C@@H]1CC#N] | 4-((S)-4-acryloyl-3- (cyanomethyl)piperazin-1-yl)-2-(2- (dimethylamino)propoxy)-7-(8- methylnaphthalen-1-yl)-5,6,7,8- tetrahydro-1,7-naphthyridine-3- carbonitrile; | 578 |
| 374 | 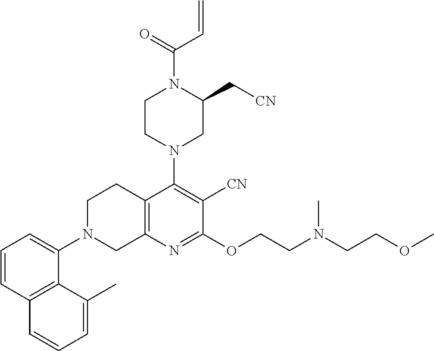 [SMILES: C=CC(=O)N1CCN(c2c(C#N)c(OCCN(C)CCOC)nc3c2CCN(c2cccc4cccc(C)c24)C3)C[C@@H]1CC#N] | (S)-4-(4-acryloyl-3- (cyanomethyl)piperazin-1-yl)-2-(2- ((2- methoxyethyl)(methyl)amino)ethoxy)- 7-(8-methylnaphthalen-1-yl)-5,6,7,8- tetrahydro-1,7-naphthyridine-3- carbonitrile | 608 |
| 375 | 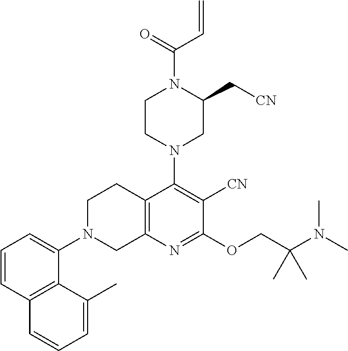 [SMILES: C=CC(=O)N1CCN(c2c(C#N)c(OCC(C)(C)N(C)C)nc3c2CCN(c2cccc4cccc(C)c24)C3)C[C@@H]1CC#N] | (S)-4-(4-acryloyl-3- (cyanomethyl)piperazin-1-yl)-2-(2- (dimethylamino)-2-methylpropoxy)-7- (8-methylnaphthalen-1-yl)-5,6,7,8- tetrahydro-1,7-naphthyridine-3- carbonitrile; | 592 |
| 376 | 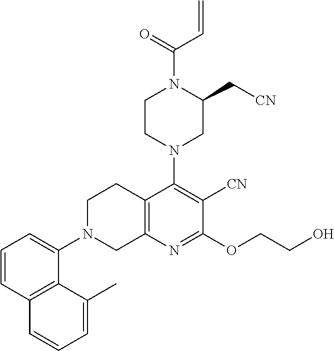 [SMILES: C=CC(=O)N1CCN(c2c(C#N)c(OCCO)nc3c2CCN(c2cccc4cccc(C)c24)C3)C[C@@H]1CC#N] | (S)-4-(4-acryloyl-3- (cyanomethyl)piperazin-1-yl)-2-(2- hydroxyethoxy)-7-(8- methylnaphthalen-1-yl)-5,6,7,8- tetrahydro-1,7-naphthyridine-3- carbonitrile; | 537 |
| 377 | 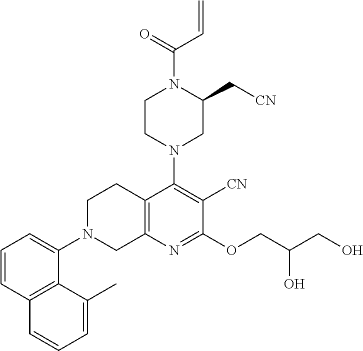 [SMILES: C=CC(=O)N1CCN(c2c(C#N)c(OCC(O)CO)nc3c2CCN(c2cccc4cccc(C)c24)C3)C[C@@H]1CC#N] | 4-((S)-4-acryloyl-3- (cyanomethyl)piperazin-1-yl)-2-(2,3- dihydroxypropoxy)-7-(8- methylnaphthalen-1-yl)-5,6,7,8- tetrahydro-1,7-naphthyridine-3- carbonitrile; | 567 |
| 378 | 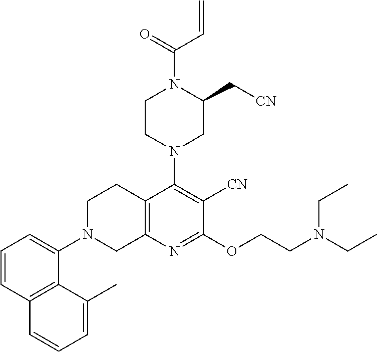 [SMILES: C=CC(=O)N1CCN(c2c(C#N)c(OCCN(CC)CC)nc3c2CCN(c2cccc4cccc(C)c24)C3)C[C@@H]1CC#N] | (S)-4-(4-acryloyl-3- (cyanomethyl)piperazin-1-yl)-2-(2- (diethylamino)ethoxy)-7-(8- methylnaphthalen-1-yl)-5,6,7,8- tetrahydro-1,7-naphthyridine-3- carbonitrile; | 592 |
| 379 | 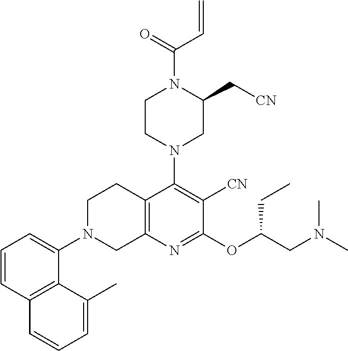 [SMILES: C=CC(=O)N1CCN(c2c(C#N)c(O[C@H](CC)CN(C)C)nc3c2CCN(c2cccc4cccc(C)c24)C3)C[C@@H]1CC#N] | 4-((S)-4-acryloyl-3- (cyanomethyl)piperazin-1-yl)-2-(((R)- 1-(dimethylamino)butan-2-yl)oxy)-7- (8-methylnaphthalen-1-yl)-5,6,7,8- tetrahydro-1,7-naphthyridine-3- carbonitrile; | 592 |
| 380 | 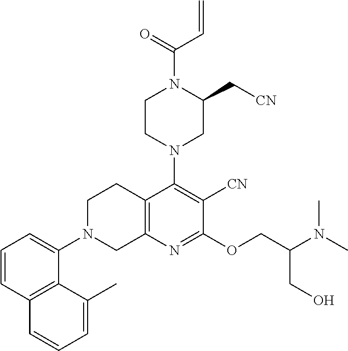 [SMILES: C=CC(=O)N1CCN(c2c(C#N)c(OCC(CO)N(C)C)nc3c2CCN(c2cccc4cccc(C)c24)C3)C[C@@H]1CC#N] | 4-((S)-4-acryloyl-3- (cyanomethyl)piperazin-1-yl)-2-(2- (dimethylamino)-3-hydroxyl)ropoxy)- 7-(8-methylnaphthalen-1-yl)-5,6,7,8- tetrahydro-1,7-naphthyridine-3- carbonitrile; | 594 |
| 381 | 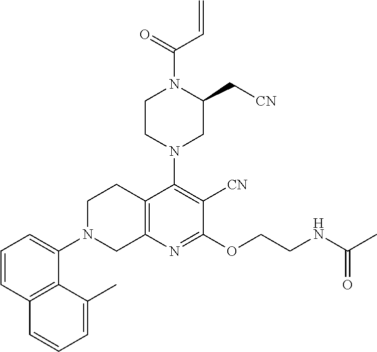 [SMILES: C=CC(=O)N1CCN(c2c(C#N)c(OCCNC(C)=O)nc3c2CCN(c2cccc4cccc(C)c24)C3)C[C@@H]1CC#N] | (S)-N-(2-((4-(4-acryloyl-3- (cyanomethyl)piperazin-1-yl)-3- cyano-7-(8-methylnaphthalen-1-yl)- 5,6,7,8-tetrahydro-1,7-naphthyridin-2- yl)oxy)ethyl)acetamide; | 578 |
| 382 | 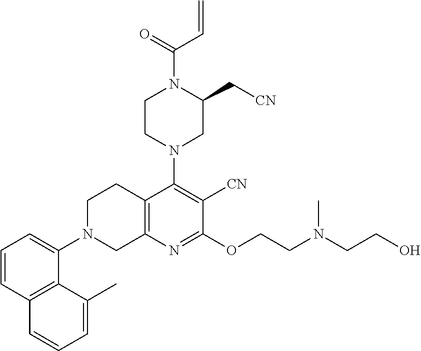 [SMILES: C=CC(=O)N1CCN(c2c(C#N)c(OCCN(C)CCO)nc3c2CCN(c2cccc4cccc(C)c24)C3)C[C@@H]1CC#N] | (S)-4-(4-acryloyl-3- (cyanomethyl)piperazin-1-yl)-2-(2- ((2- hydroxyethyl)(methyl)amino)ethoxy)- 7-(8-methylnaphthalen-1-yl)-5,6,7,8- tetrahydro-1,7-naphthyridine-3- carbonitrile; | 594 |
| 383 | 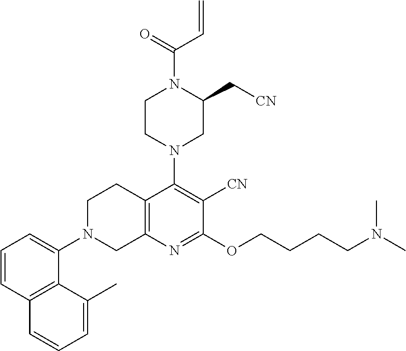 [SMILES: C=CC(=O)N1CCN(c2c(C#N)c(OCCCCN(C)C)nc3c2CCN(c2cccc4cccc(C)c24)C3)C[C@@H]1CC#N] | (S)-4-(4-acryloyl-3- (cyanomethyl)piperazin-1-yl)-2-(4- (dimethylamino)butoxy)-7-(8- methylnaphthalen-1-yl)-5,6,7,8- tetrahydro-1,7-naphthyridine-3- carbonitrile; | 592 |
| 384 | 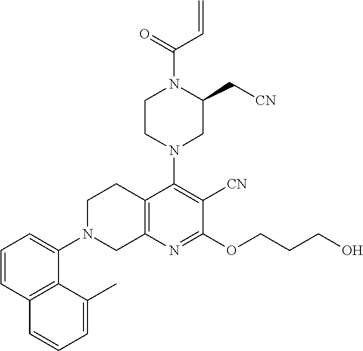 [SMILES: C=CC(=O)N1CCN(c2c(C#N)c(OCCCO)nc3c2CCN(c2cccc4cccc(C)c24)C3)C[C@@H]1CC#N] | (S)-4-(4-acryloyl-3- (cyanomethyl)piperazin-1-yl)-2-(3- hydroxypropoxy)-7-(8- methylnaphthalen-1-yl)-5,6,7,8- tetrahydro-1,7-naphthyridine-3- carbonitrile; | 551 |
| 385 | 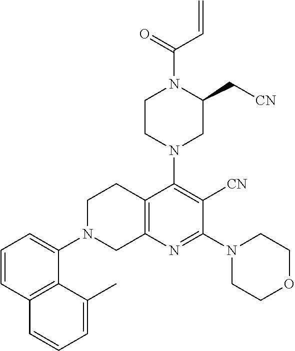 [SMILES: C=CC(=O)N1CCN(c2c(C#N)c(N3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2- morpholino-5,6,7,8-tetrahydro-1,7- naphthyridine-3-carbonitrile; | 562 |
| 386 | 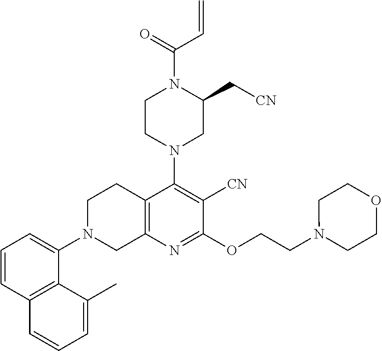 [SMILES: C=CC(=O)N1CCN(c2c(C#N)c(OCCN3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2- morpholinoethoxy)-5,6,7,8- tetrahydro-1,7-naphthyridine-3- carbonitrile; | 606 |
| 387 | 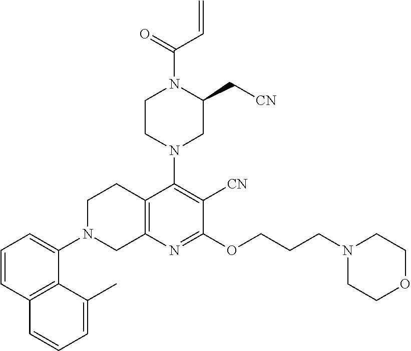 [SMILES: C=CC(=O)N1CCN(c2c(C#N)c(OCCCN3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- morpholinopropoxy)-5,6,7,8- tetrahydro-1,7-naphthyridine-3- carbonitrile; | 620 |
| 388 | 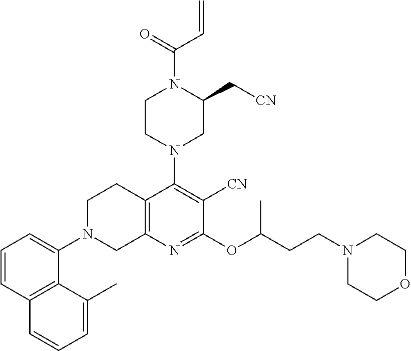 [SMILES: C=CC(=O)N1CCN(c2c(C#N)c(OC(C)CCN3CCOCC3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((4- morpholinobutan-2-yl)oxy)-5,6,7,8- tetrahydro-1,7-naphthyridine-3- carbonitrile; | 634 |
| 389 | 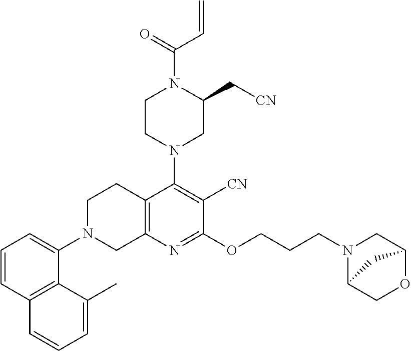 [SMILES: C=CC(=O)N1CCN(c2c(C#N)c(OCCCN3C[C@@H]4C[C@H]3CO4)nc3c2CCN(c2cccc4cccc(C)c24)C3)C[C@@H]1CC#N] | 2-(3-((1S,4S)-2-oxa-5- azabicyclo[2.2.1]heptan-5- yl)propoxy)-4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 632 |
| 390 | 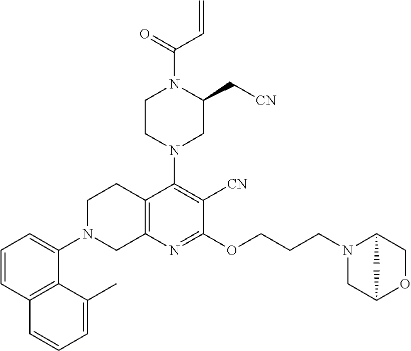 [SMILES: C=CC(=O)N1CCN(c2c(C#N)c(OCCCN3C[C@H]4C[C@@H]3CO4)nc3c2CCN(c2cccc4cccc(C)c24)C3)C[C@@H]1CC#N] | 2-(3-((1R,4R)-2-oxa-5- azabicyclo[2.2.1]heptan-5- yl)propoxy)-4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 632 |
| 391 | 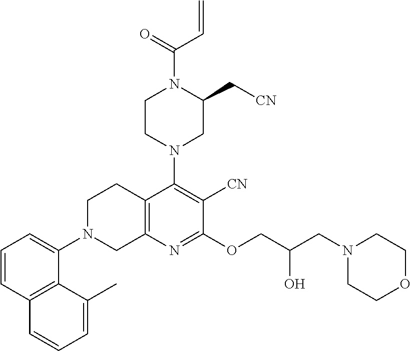 [SMILES: C=CC(=O)N1CCN(c2c(C#N)c(OCC(O)CN3CCOCC3)nc3c2CCN(c2cccc4cccc(C)c24)C3)C[C@@H]1CC#N] | 4-((S)-4-acryloyl-3- (cyanomethyl)piperazin-1-yl)-2-(2- hydroxy-3-morpholinopropoxy)-7-(8- methylnaphthalen-1-yl)-5,6,7,8- tetrahydro-1,7-naphthyridine-3- carbonitrile; | 636 |
| 392 | 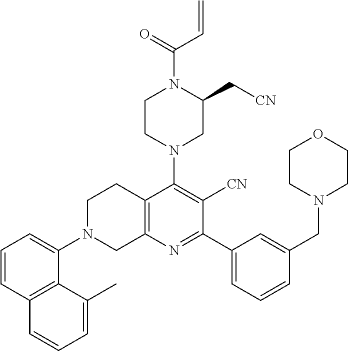 [SMILES: C=CC(=O)N1CCN(c2c(C#N)c(-c3cccc(CN4CCOCC4)c3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3- (morpholinomethyl)phenyl)-5,6,7,8- tetrahydro-1,7-naphthyridine-3- carbonitrile; | 652 |
| 393 | 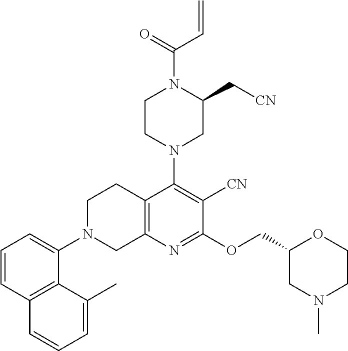 [SMILES: C=CC(=O)N1CCN(c2c(C#N)c(OC[C@H]3CN(C)CCO3)nc3c2CCN(c2cccc4cccc(C)c24)C3)C[C@@H]1CC#N] | 4-((S)-4-acryloyl-3- (cyanomethyl)piperazin-1-yl)-2-(((R)- 4-methylmorpholin-2-yl)methoxy)-7- (8-methylnaphthalen-1-yl)-5,6,7,8- tetrahydro-1,7-naphthyridine-3- carbonitrile; | 606 |
| 394 | 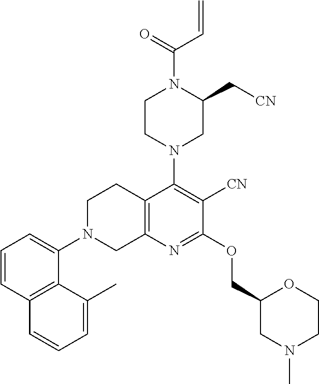 [SMILES: C=CC(=O)N1CCN(c2c(C#N)c(OC[C@@H]3CN(C)CCO3)nc3c2CCN(c2cccc4cccc(C)c24)C3)C[C@@H]1CC#N] | 4-((S)-4-acryloyl-3- (cyanomethyl)piperazin-1-yl)-2-(((S)- 4-methylmorpholin-2-yl)methoxy)-7- (8-methylnaphthalen-1-yl)-5,6,7,8- tetrahydro-1,7-naphthyridine-3- carbonitrile; | 606 |
| 395 | 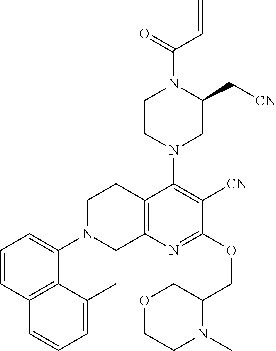 [SMILES: C=CC(=O)N1CCN(c2c(C#N)c(OCC3COCCN3C)nc3c2CCN(c2cccc4cccc(C)c24)C3)C[C@@H]1CC#N] | 4-((S)-4-acryloyl-3- (cyanomethyl)piperazin-1-yl)-2-((4- methylmorpholin-3-yl)methoxy)-7-(8- methylnaphthalen-1-yl)-5,6,7,8- tetrahydro-1,7-naphthyridine-3- carbonitrile; | 606 |
| 396 | 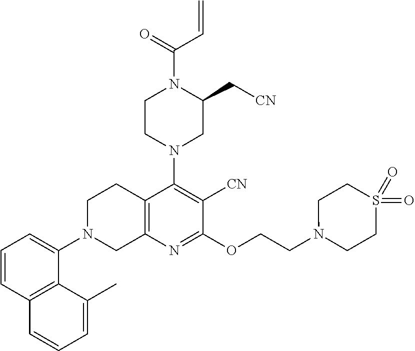 [SMILES: C=CC(=O)N1CCN(c2c(C#N)c(OCCN3CCS(=O)(=O)CC3)nc3c2CCN(c2cccc4cccc(C)c24)C3)C[C@@H]1CC#N] | (S)-4-(4-acryloyl-3- (cyanomethyl)piperazin-1-yl)-2-(2- (1,1-dioxidothiomorpholino)ethoxy)- 7-(8-methylnaphthalen-1-yl)-5,6,7,8- tetrahydro-1,7-naphthyridine-3- carbonitrile; | 654 |
| 397 | 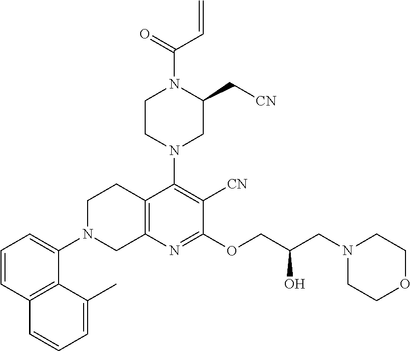 [SMILES: C=CC(=O)N1CCN(c2c(C#N)c(OC[C@H](O)CN3CCOCC3)nc3c2CCN(c2cccc4cccc(C)c24)C3)C[C@@H]1CC#N] | 4-((S)-4-acryloyl-3- (cyanomethyl)piperazin-1-yl)-2-((R)- 2-hydroxy-3-morpholinopropoxy)-7- (8-methylnaphthalen-1-yl)-5,6,7,8- tetrahydro-1,7-naphthyridine-3- carbonitrile; | 636 |
| 398 | 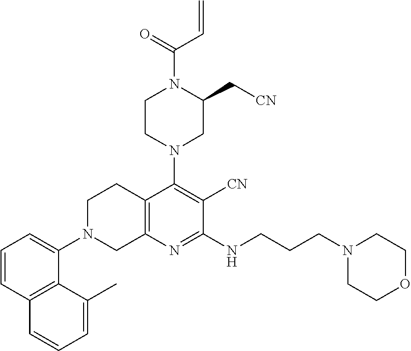 [SMILES: C=CC(=O)N1CCN(c2c(C#N)c(NCCCN3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((3- morpholinopropyl)amino)-5,6,7,8- tetrahydro-1,7-naphthyridine-3- carbonitrile; | 619 |
| 399 | 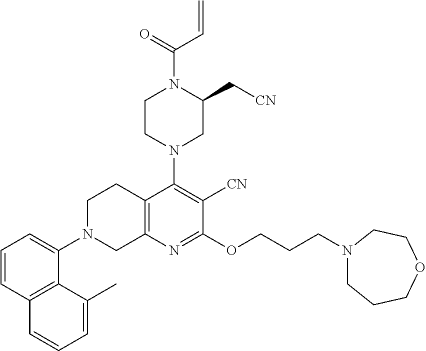 [SMILES: C=CC(=O)N1CCN(c2c(C#N)c(OCCCN3CCCOCC3)nc3c2CCN(c2cccc4cccc(C)c24)C3)C[C@@H]1CC#N] | (S)-2-(3-(1,4-oxazepan-4- yl)propoxy)-4-(4-acryloyl-3- (cyanomethyl)piperazin-1-yl)-7-(8- methylnaphthalen-1-yl)-5,6,7,8- tetrahydro-1,7-naphthyridine-3- carbonitrile; | 634 |
| 400 | 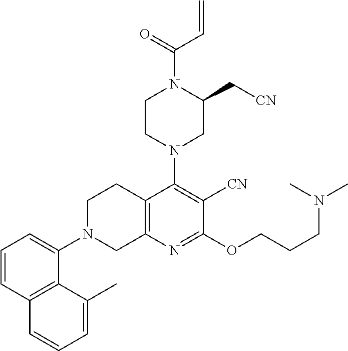 [SMILES: C=CC(=O)N1CCN(c2c(C#N)c(OCCCN(C)C)nc3c2CCN(c2cccc4cccc(C)c24)C3)C[C@@H]1CC#N] | (S)-4-(4-acryloyl-3- (cyanomethyl)piperazin-1-yl)-2-(3- (dimethylamino)propoxy)-7-(8- methylnaphthalen-1-yl)-5,6,7,8- tetrahydro-1,7-naphthyridine-3- carbonitrile; | 578 |
| 401 | 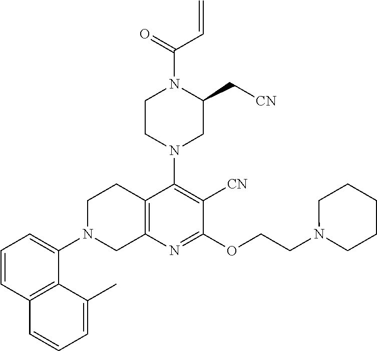 [SMILES: C=CC(=O)N1CCN(c2c(C#N)c(OCCN3CCCCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2- (piperidin-1-yl)ethoxy)-5,6,7,8- tetrahydro-1,7-naphthyridine-3- carbonitrile; | 604 |
| 402 | 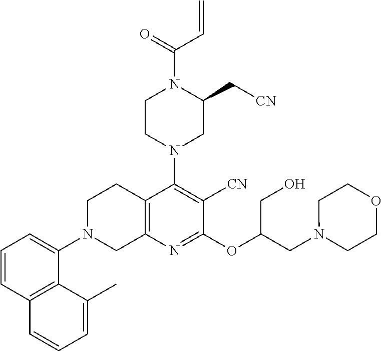 [SMILES: C=CC(=O)N1CCN(c2c(C#N)c(OC(CO)CN3CCOCC3)nc3c2CCN(c2cccc4cccc(C)c24)C3)C[C@@H]1CC#N] | 4-((S)-4-acryloyl-3- (cyanomethyl)piperazin-1-yl)-2-((1- hydroxy-3-morpholinopropan-2- yl)oxy)-7-(8-methylnaphthalen-1-yl)- 5,6,7,8-tetrahydro-1,7-naphthyridine- 3-carbonitrile; | 636 |
| 403 | 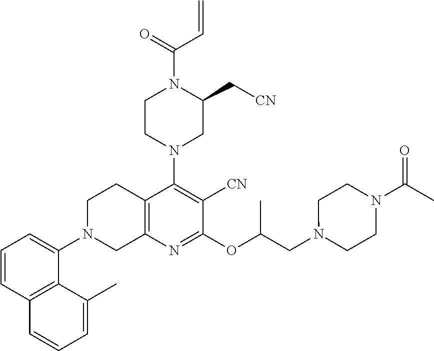 [SMILES: C=CC(=O)N1CCN(c2c(C#N)c(OC(C)CN3CCN(C(C)=O)CC3)nc3c2CCN(c2cccc4cccc(C)c24)C3)C[C@@H]1CC#N] | 2-((1-(4-acetylpiperazin-1-yl)propan- 2-yl)oxy)-4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 661 |
| 404 | 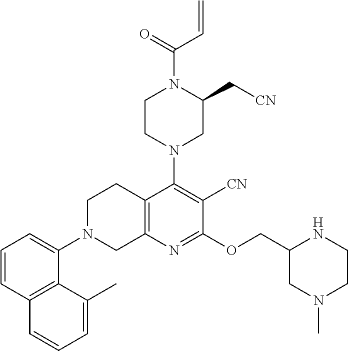 [SMILES: C=CC(=O)N1CCN(c2c(C#N)c(OCC3CN(C)CCN3)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((4- methylpiperazin-2-yl)methoxy)- 5,6,7,8-tetrahydro-1,7-naphthyridine- 3-carbonitrile; | 605 |
| 405 | 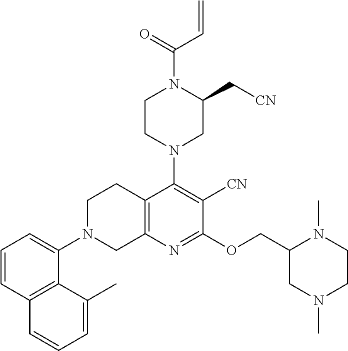 [SMILES: C=CC(=O)N1CCN(c2c(C#N)c(OCC3CN(C)CCN3C)nc3c2CCN(c2cccc4cccc(C)c24)C3)C[C@@H]1CC#N] | 4-((S)-4-acryloyl-3- (cyanomethyl)piperazin-1-yl)-2-((1,4- dimethylpiperazin-2-yl)methoxy)-7- (8-methylnaphthalen-1-yl)-5,6,7,8- tetrahydro-1,7-naphthyridine-3- carbonitrile; | 619 |
| 406 | 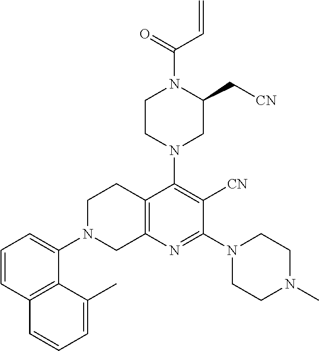 [SMILES: C=CC(=O)N1CCN(c2c(C#N)c(N3CCN(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(4- methylpiperazin-1-yl)-5,6,7,8- tetrahydro-1,7-naphthyridine-3- carbonitrile; | 575 |
| 407 | 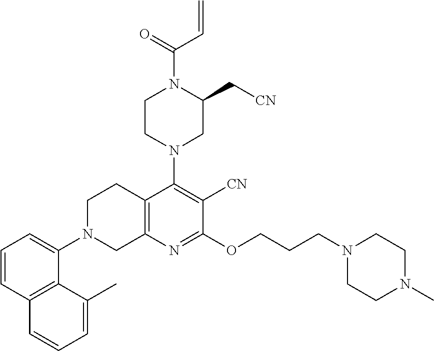 [SMILES: C=CC(=O)N1CCN(c2c(C#N)c(OCCCN3CCN(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(3-(4- methylpiperazin-1-yl)propoxy)- 5,6,7,8-tetrahydro-1,7-naphthyridine- 3-carbonitrile; | 633 |
| 408 | 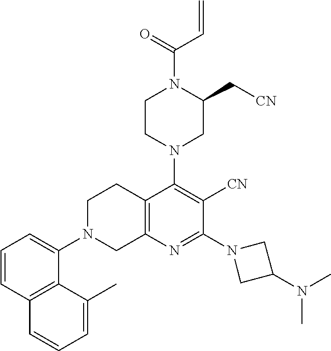 [SMILES: C=CC(=O)N1CCN(c2c(C#N)c(N3CC(N(C)C)C3)nc3c2CCN(c2cccc4cccc(C)c24)C3)C[C@@H]1CC#N] | (S)-4-(4-acryloyl-3- (cyanomethyl)piperazin-1-yl)-2-(3- (dimethylamino)azetidin-1-yl)-7-(8- methylnaphthalen-1-yl)-5,6,7,8- tetrahydro-1,7-naphthyridine-3- carbonitrile; | 575 |
| 409 | 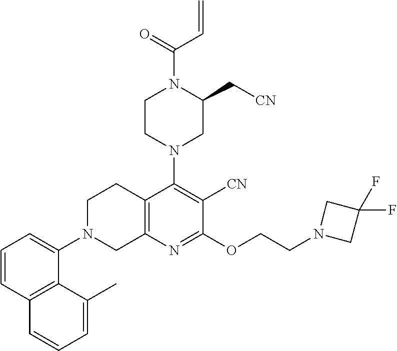 [SMILES: C=CC(=O)N1CCN(c2c(C#N)c(OCCN3CC(F)(F)C3)nc3c2CCN(c2cccc4cccc(C)c24)C3)C[C@@H]1CC#N] | (S)-4-(4-acryloyl-3- (cyanomethyl)piperazin-1-yl)-2-(2- (3,3-difluoroazetidin-1-yl)ethoxy)-7- (8-methylnaphthalen-1-yl)-5,6,7,8- tetrahydro-1,7-naphthyridine-3- carbonitrile; | 612 |
| 410 | 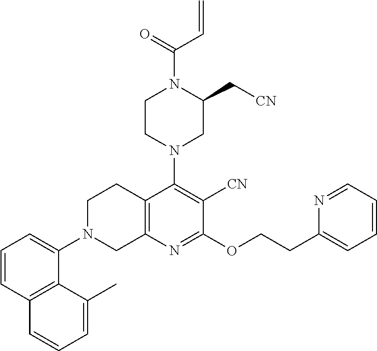 [SMILES: C=CC(=O)N1CCN(c2c(C#N)c(OCCc3ccccn3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2-(pyridin- 2-yl)ethoxy)-5,6,7,8-tetrahydro-1,7- naphthyridine-3-carbonitrile; | 598 |
| 411 | 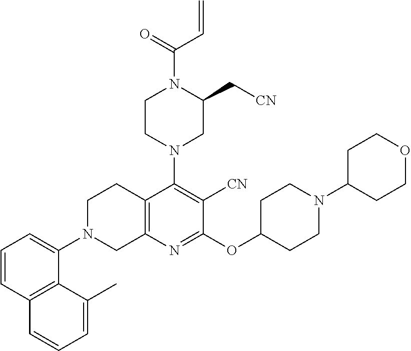 [SMILES: C=CC(=O)N1CCN(c2c(C#N)c(OC3CCN(C4CCOCC4)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- (tetrahydro-2H-pyran-4-yl)piperidin- 4-yl)oxy)-5,6,7,8-tetrahydro-1,7- naphthyridine-3-carbonitrile; | 660 |
| 412 | 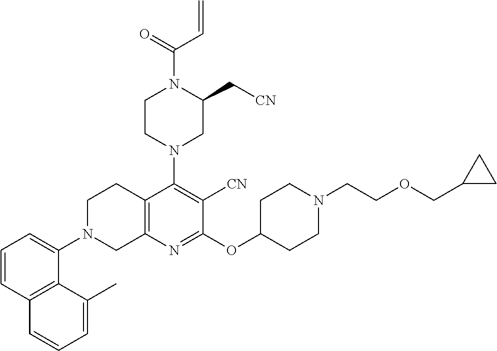 [SMILES: C=CC(=O)N1CCN(c2c(C#N)c(OC3CCN(CCOCC4CC4)CC3)nc3c2CCN(c2cccc4cccc(C)c24)C3)C[C@@H]1CC#N] | (S)-4-(4-acryloyl-3- (cyanomethyl)piperazin-1-yl)-2-((1- (2- (cyclopropylmethoxy)ethyl)piperidin- 4-yl)oxy)-7-(8-methylnaphthalen-1- yl)-5,6,7,8-tetrahydro-1,7- naphthyridine-3-carbonitrile; | 674 |
| 413 | 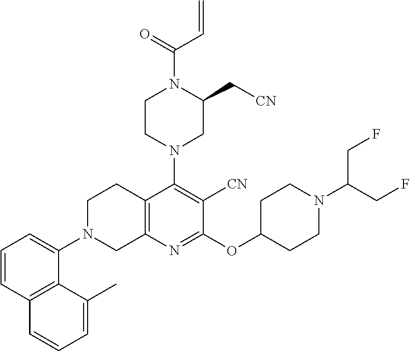 [SMILES: C=CC(=O)N1CCN(c2c(C#N)c(OC3CCN(C(CF)CF)CC3)nc3c2CCN(c2cccc4cccc(C)c24)C3)C[C@@H]1CC#N] | (S)-4-(4-acryloyl-3- (cyanomethyl)piperazin-1-yl)-2-((1- (1,3-difluoropropan-2-yl)piperidin-4- yl)oxy)-7-(8-methylnaphthalen-1-yl)- 5,6,7,8-tetrahydro-1,7-naphthyridine- 3-carbonitrile | 654 |
| 414 | 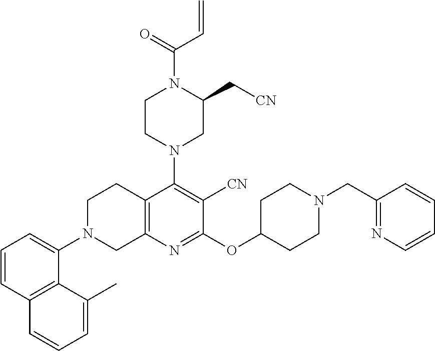 [SMILES: C=CC(=O)N1CCN(c2c(C#N)c(OC3CCN(Cc4ccccn4)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- (pyridin-2-ylmethyl)piperidin-4- yl)oxy)-5,6,7,8-tetrahydro-1,7- naphthyridine-3-carbonitrile; | 667 |
| 415 | 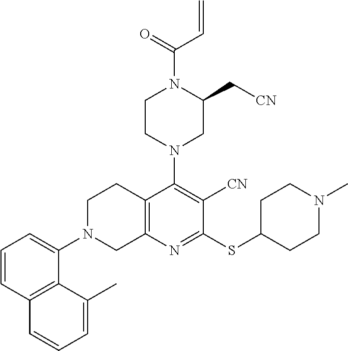 [SMILES: C=CC(=O)N1CCN(c2c(C#N)c(SC3CCN(C)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- methylpiperidin-4-yl)thio)-5,6,7,8- tetrahydro-1,7-naphthyridine-3- carbonitrile; | 606 |
| 416 | 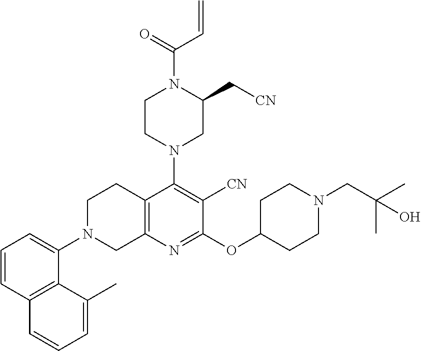 [SMILES: C=CC(=O)N1CCN(c2c(C#N)c(OC3CCN(CC(C)(C)O)CC3)nc3c2CCN(c2cccc4cccc(C)c24)C3)C[C@@H]1CC#N] | (S)-4-(4-acryloyl-3- (cyanomethyl)piperazin-1-yl)-2-((1- (2-hydroxy-2-methylpropyl)piperidin- 4-yl)oxy)-7-(8-methylnaphthalen-1- yl)-5,6,7,8-tetrahydro-1,7- naphthyridine-3-carbonitrile; | 648 |
| 417 | 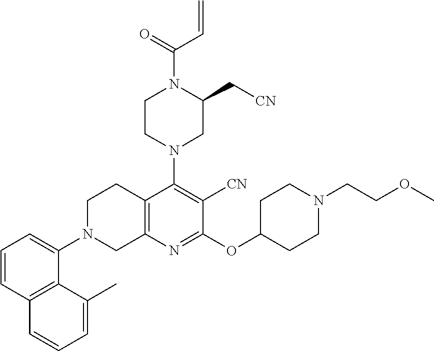 [SMILES: C=CC(=O)N1CCN(c2c(C#N)c(OC3CCN(CCOC)CC3)nc3c2CCN(c2cccc4cccc(C)c24)C3)C[C@@H]1CC#N] | (S)-4-(4-acryloyl-3- (cyanomethyl)piperazin-1-yl)-2-((1- (2-methoxyethyl)piperidin-4-yl)oxy)- 7-(8-methylnaphthalen-1-yl)-5,6,7,8- tetrahydro-1,7-naphthyridine-3- carbonitrile; | 634 |
| 418 | 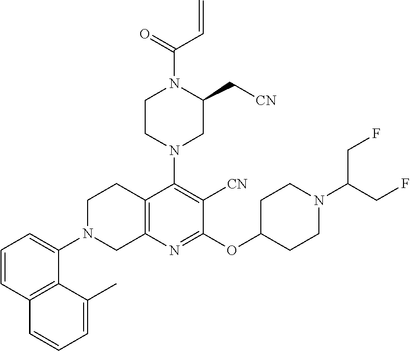 [SMILES: C=CC(=O)N1CCN(c2c(C#N)c(OC3CCN(C(CF)CF)CC3)nc3c2CCN(c2cccc4cccc(C)c24)C3)C[C@@H]1CC#N] | (S)-4-(4-acryloyl-3- (cyanomethyl)piperazin-1-yl)-2-((1- (1,3-difluoropropan-2-yl)piperidin-4- yl)oxy)-7-(8-methylnaphthalen-1-yl)- 5,6,7,8-tetrahydro-1,7-naphthyridine- 3-carbonitrile; | 654 |
| 419 | 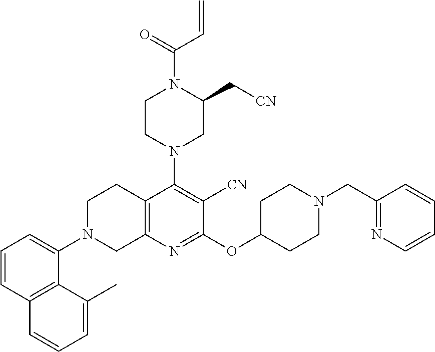 [SMILES: C=CC(=O)N1CCN(c2c(C#N)c(OC3CCN(Cc4ccccn4)CC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-((1- (pyridin-2-ylmethyl)piperidin-4- yl)oxy)-5,6,7,8-tetrahydro-1,7- naphthyridine-3-carbonitrile; | 667 |
| 420 | 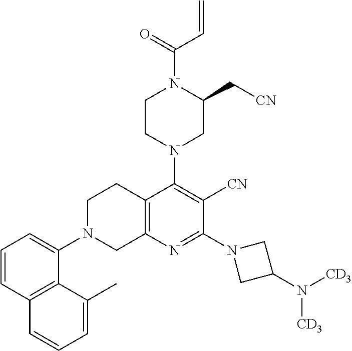 [SMILES: [2H]C([2H])([2H])N(C1CN(c2nc3c(c(N4CCN(C(=O)C=C)[C@@H](CC#N)C4)c2C#N)CCN(c2cccc4cccc(C)c24)C3)C1)C([2H])([2H])[2H]] | (S)-4-(4-acryloyl-3- (cyanomethyl)piperazin-1-yl)-2-(3- (bis(methyl-d3)amino)azetidin-1-yl)- 7-(8-methylnaphthalen-1-yl)-5,6,7,8- tetrahydro-1,7-naphthyridine-3- carbonitrile; | 581 |
| 421 | 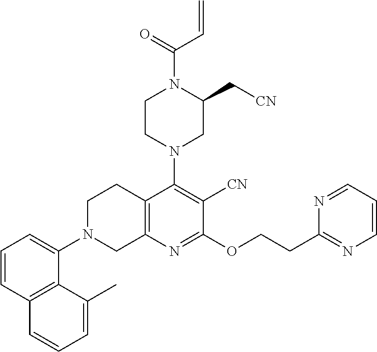 [SMILES: C=CC(=O)N1CCN(c2c(C#N)c(OCCc3ncccn3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2- (pyrimidin-2-yl)ethoxy)-5,6,7,8- tetrahydro-1,7-naphthyridine-3- carbonitrile; | 599 |
| 422 | 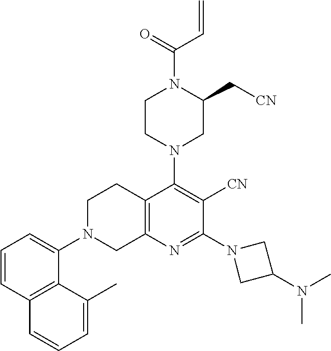 [SMILES: C=CC(=O)N1CCN(c2c(C#N)c(N3CC(N(C)C)C3)nc3c2CCN(c2cccc4cccc(C)c24)C3)C[C@@H]1CC#N] | (S)-4-(4-acryloyl-3- (cyanomethyl)piperazin-1-yl)-2-(3- (dimethylamino)azetidin-1-yl)-7-(8- methylnaphthalen-1-yl)-5,6,7,8- tetrahydro-1,7-naphthyridine-3- carbonitrile; | 575 |
| 423 | 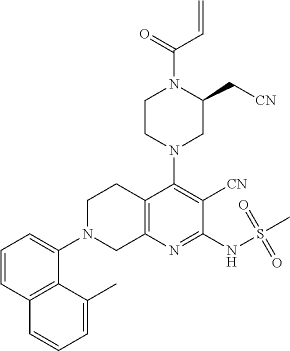 [SMILES: C=CC(=O)N1CCN(c2c(C#N)c(NS(C)(=O)=O)nc3c2CCN(c2cccc4cccc(C)c24)C3)C[C@@H]1CC#N] | (S)-N-(4-(4-acryloyl-3- (cyanomethyl)piperazin-1-yl)-3- cyano-7-(8-methylnaphthalen-1-yl)- 5,6,7,8-tetrahydro-1,7-naphthyridin-2- yl)methanesulfonamide; | 570 |
| 424 | 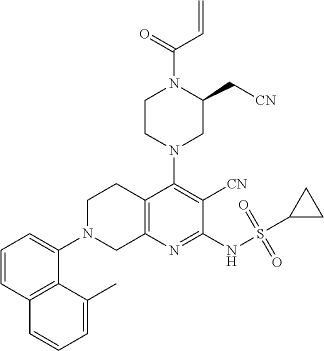 [SMILES: C=CC(=O)N1CCN(c2c(C#N)c(NS(=O)(=O)C3CC3)nc3c2CCN(c2cccc4cccc(C)c24)C3)C[C@@H]1CC#N] | (S)-N-(4-(4-acryloyl-3- (cyanomethyl)piperazin-1-yl)-3- cyano-7-(8-methylnaphthalen-1-yl)- 5,6,7,8-tetrahydro-1,7-naphthyridin-2- yl)cyclopropanesulfonamide; | 596 |
| 425 | 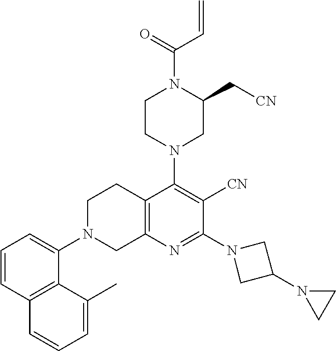 [SMILES: C=CC(=O)N1CCN(c2c(C#N)c(N3CC(N4CC4)C3)nc3c2CCN(c2cccc4cccc(C)c24)C3)C[C@@H]1CC#N] | (S)-4-(4-acryloyl-3- (cyanomethyl)piperazin-1-yl)-2-(3- (aziridin-1-yl)azetidin-1-yl)-7-(8- methylnaphthalen-1-yl)-5,6,7,8- tetrahydro-1,7-naphthyridine-3- carbonitrile; | 573 |
| 426 | 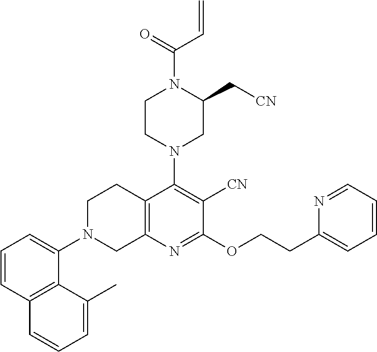 [SMILES: C=CC(=O)N1CCN(c2c(C#N)c(OCCc3ccccn3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(2-(pyridin- 2-yl)ethoxy)-5,6,7,8-tetrahydro-1,7- naphthyridine-3-carbonitrile; | 598 |
| 427 | 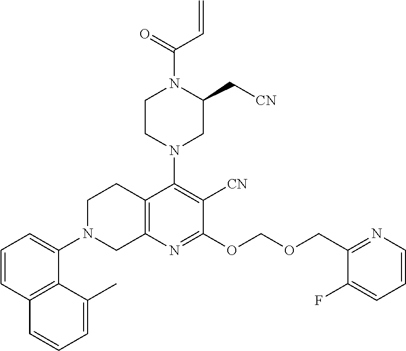 [SMILES: C=CC(=O)N1CCN(c2c(C#N)c(OCOCc3ncccc3F)nc3c2CCN(c2cccc4cccc(C)c24)C3)C[C@@H]1CC#N] | (S)-4-(4-acryloyl-3- (cyanomethyl)piperazin-1-yl)-2-(((3- fluoropyridin-2- yl)methoxy)methoxy)-7-(8- methylnaphthalen-1-yl)-5,6,7,8- tetrahydro-1,7-naphthyridine-3- carbonitrile; | 632 |
| 428 | 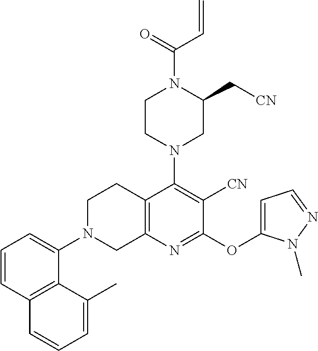 [SMILES: C=CC(=O)N1CCN(c2c(C#N)c(Oc3ccnn3C)nc3c2CCN(c2cccc4cccc(C)c24)C3)C[C@@H]1CC#N] | (S)-4-(4-acryloyl-3- (cyanomethyl)piperazin-1-yl)-2-((1- methyl-1H-pyrazol-5-yl)oxy)-7-(8- methylnaphthalen-1-yl)-5,6,7,8- tetrahydro-1,7-naphthyridine-3- carbonitrile; | 573 |
| 429 | 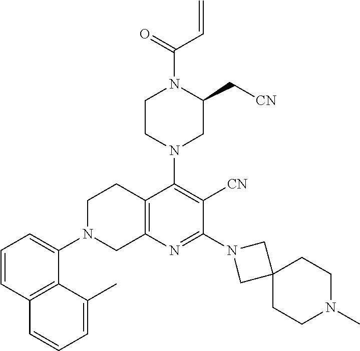 [SMILES: C=CC(=O)N1CCN(c2c(C#N)c(N3CC4(CCN(C)CC4)C3)nc3c2CCN(c2cccc4cccc(C)c24)C3)C[C@@H]1CC#N] | (S)-4-(4-acryloyl-3- (cyanomethyl)piperazin-1-yl)-2-(7- methyl-2,7-diazaspiro[3.5]nonan-2- yl)-7-(8-methylnaphthalen-1-yl)- 5,6,7,8-tetrahydro-1,7-naphthyridine- 3-carbonitrile; | 615 |
| 430 | 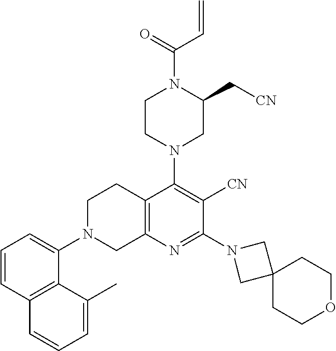 [SMILES: C=CC(=O)N1CCN(c2c(C#N)c(N3CC4(CCOCC4)C3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(7-oxa-2- azaspiro[3.5]nonan-2-yl)-5,6,7,8- tetrahydro-1,7-naphthyridine-3- carbonitrile; | 602 |
| 431 | 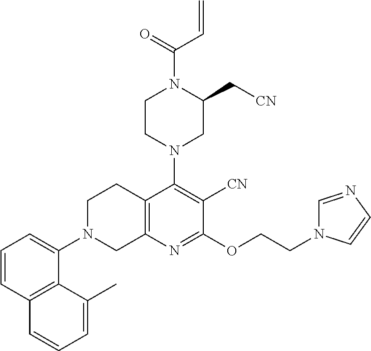 [SMILES: C=CC(=O)N1CCN(c2c(C#N)c(OCCn3ccnc3)nc3c2CCN(c2cccc4cccc(C)c24)C3)C[C@@H]1CC#N] | (S)-2-(2-(1H-imidazol-1-yl)ethoxy)-4- (4-acryloyl-3-(cyanomethyl)piperazin- 1-yl)-7-(8-methylnaphthalen-1-yl)- 5,6,7,8-tetrahydro-1,7-naphthyridine- 3-carbonitrile; | 587 |
| 432 | 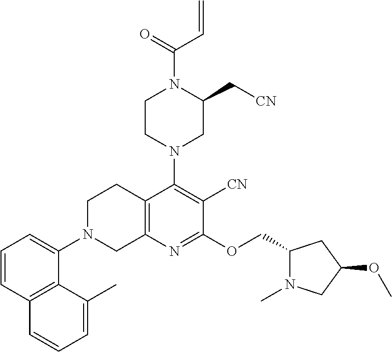 [SMILES: C=CC(=O)N1CCN(c2c(C#N)c(OC[C@@H]3C[C@@H](OC)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4R)-4-methoxy-1- methylpyrrolidin-2-yl)methoxy)-7-(8- methylnaphthalen-1-yl)-5,6,7,8- tetrahydro-1,7-naphthyridine-3- carbonitrile; | 620 |
| 433 | 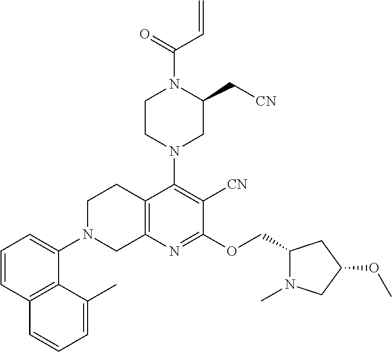 [SMILES: C=CC(=O)N1CCN(c2c(C#N)c(OC[C@@H]3C[C@H](OC)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4S)-4-methoxy-1- methylpyrrolidin-2-yl)methoxy)-7-(8- methylnaphthalen-1-yl)-5,6,7,8- tetrahydro-1,7-naphthyridine-3- carbonitrile; | 620 |
| 434 | 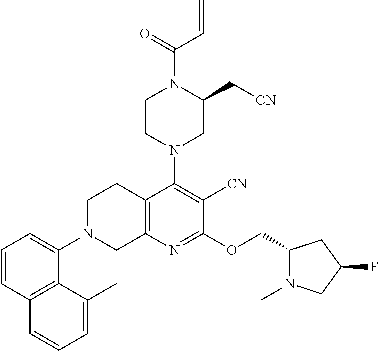 [SMILES: C=CC(=O)N1CCN(c2c(C#N)c(OC[C@@H]3C[C@@H](F)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4R)-4-fluoro-1- methylpyrrolidin-2-yl)methoxy)-7-(8- methylnaphthalen-1-yl)-5,6,7,8- tetrahydro-1,7-naphthyridine-3- carbonitrile; | 608 |
| 435 | 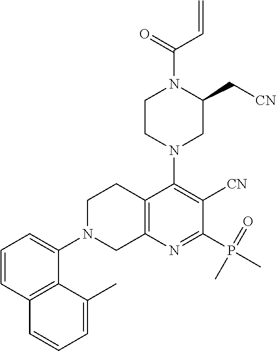 [SMILES: C=CC(=O)N1CCN(c2c(C#N)c(P(C)(C)=O)nc3c2CCN(c2cccc4cccc(C)c24)C3)C[C@@H]1CC#N] | (S)-4-(4-acryloyl-3- (cyanomethyl)piperazin-1-yl)-2- (dimethylphosphoryl)-7-(8- methylnaphthalen-1-yl)-5,6,7,8- tetrahydro-1,7-naphthyridine-3- carbonitrile; | 553 |
| 436 | 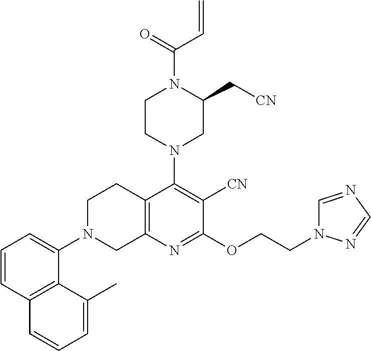 [SMILES: C=CC(=O)N1CCN(c2c(C#N)c(OCCn3cncn3)nc3c2CCN(c2cccc4cccc(C)c24)C3)C[C@@H]1CC#N] | (S)-2-(2-(1H-1,2,4-triazol-1- yl)ethoxy)-4-(4-acryloyl-3- (cyanomethyl)piperazin-1-yl)-7-(8- methylnaphthalen-1-yl)-5,6,7,8- tetrahydro-1,7-naphthyridine-3- carbonitrile; | 588 |
| 437 | 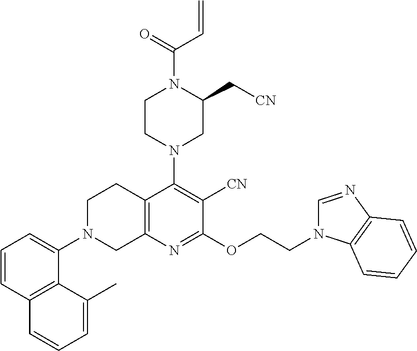 [SMILES: C=CC(=O)N1CCN(c2c(C#N)c(OCCn3cnc4ccccc43)nc3c2CCN(c2cccc4cccc(C)c24)C3)C[C@@H]1CC#N] | (S)-2-(2-(1H-benzo[d]imidazol-1- yl)ethoxy)-4-(4-acryloyl-3- (cyanomethyl)piperazin-1-yl)-7-(8- methylnaphthalen-1-yl)-5,6,7,8- tetrahydro-1,7-naphthyridine-3- carbonitrile; | 637 |
| 438 | 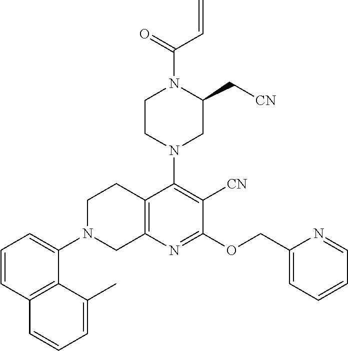 [SMILES: C=CC(=O)N1CCN(c2c(C#N)c(OCc3ccccn3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(pyridin-2- ylmethoxy)-5,6,7,8-tetrahydro-1,7- naphthyridine-3-carbonitrile; | 584 |
| 439 | 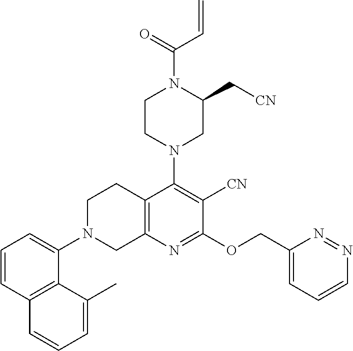 [SMILES: C=CC(=O)N1CCN(c2c(C#N)c(OCc3cccnn3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2-(pyridazin- 3-ylmethoxy)-5,6,7,8-tetrahydro-1,7- naphthyridine-3-carbonitrile; | 585 |
| 440 | 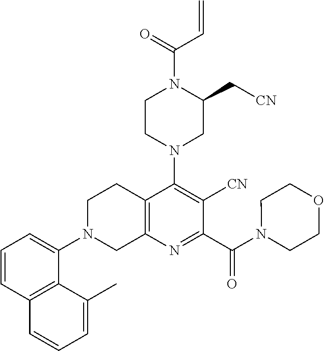 [SMILES: C=CC(=O)N1CCN(c2c(C#N)c(C(=O)N3CCOCC3)nc3c2CCN(c2cccc4cccc(C)c24)C3)C[C@@H]1CC#N] | (S)-4-(4-acryloyl-3- (cyanomethyl)piperazin-1-yl)-7-(8- methylnaphthalen-1-yl)-2- (morpholine-4-carbonyl)-5,6,7,8- tetrahydro-1,7-naphthyridine-3- carbonitrile; | 590 |
| 441 | 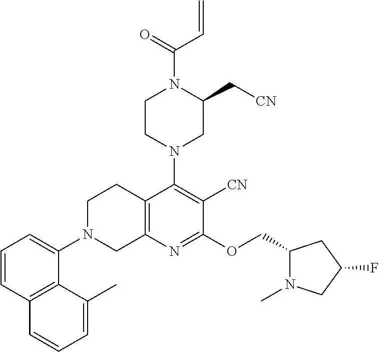 [SMILES: C=CC(=O)N1CCN(c2c(C#N)c(OC[C@@H]3C[C@H](F)CN3C)nc3c2CCN(c2cccc4cccc(C)c24)C3)C[C@@H]1CC#N] | 4-((S)-4-acryloyl-3- (cyanomethyl)piperazin-1-yl)-2- (((2S,4S)-4-fluoro-1- methylpyrrolidin-2-yl)methoxy)-7-(8- methylnaphthalen-1-yl)-5,6,7,8- tetrahydro-1,7-naphthyridine-3- carbonitrile; | 608 |
| 442 | 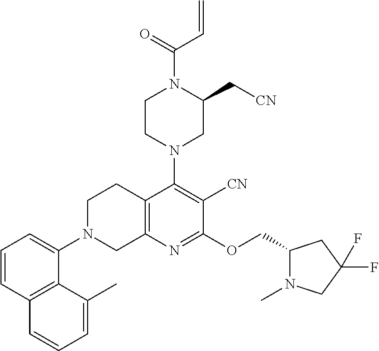 [SMILES: C=CC(=O)N1CCN(c2c(C#N)c(OC[C@@H]3CC(F)(F)CN3C)nc3c2CCN(c2cccc4cccc(C)c24)C3)C[C@@H]1CC#N] | 4-((S)-4-acryloyl-3- (cyanomethyl)piperazin-1-yl)-2-(((S)- 4,4-difluoro-1-methylpyrrolidin-2- yl)methoxy)-7-(8-methylnaphthalen- 1-yl)-5,6,7,8-tetrahydro-1,7- naphthyridine-3-carbonitrile; | 626 |
| 443 | 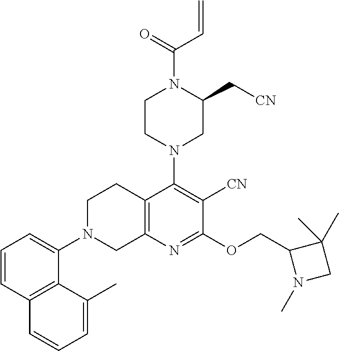 [SMILES: C=CC(=O)N1CCN(c2c(C#N)c(OCC3N(C)CC3(C)C)nc3c2CCN(c2cccc4cccc(C)c24)C3)C[C@@H]1CC#N] | 4-((S)-4-acryloyl-3- (cyanomethyl)piperazin-1-yl)-7-(8- methylnaphthalen-1-yl)-2-((1,3,3- trimethylazetidin-2-yl)methoxy)- 5,6,7,8-tetrahydro-1,7-naphthyridine- 3-carbonitrile; | 604 |
| 444 | 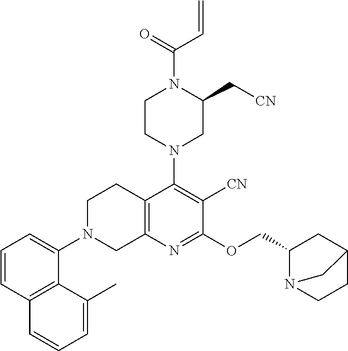 [SMILES: C=CC(=O)N1CCN(c2c(C#N)c(OC[C@@H]3CC4CCN3C4)nc3c2CCN(c2cccc4cccc(C)c24)C3)C[C@@H]1CC#N] | 2-(((2S)-1-azabicyclo[2.2.1]heptan-2- yl)methoxy)-4-((S)-4-acryloyl-3- (cyanomethyl)piperazin-1-yl)-7-(8- methylnaphthalen-1-yl)-5,6,7,8- tetrahydro-1,7-naphthyridine-3- carbonitrile; | 602 |
| 445 | 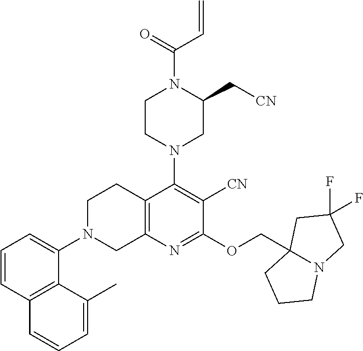 [SMILES: C=CC(=O)N1CCN(c2c(C#N)c(OCC34CCCN3CC(F)(F)C4)nc3c2CCN(c2cccc4cccc(C)c24)C3)C[C@@H]1CC#N] | 4-((S)-4-acryloyl-3- (cyanomethyl)piperazin-1-yl)-2-((2,2- difluorotetrahydro-1H-pyrrolizin- 7a(5H)-yl)methoxy)-7-(8- methylnaphthalen-1-yl)-5,6,7,8- tetrahydro-1,7-naphthyridine-3- carbonitrile; | 652 |
| 446 | 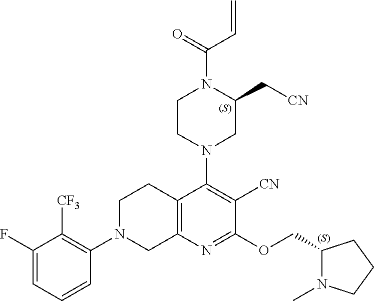 | 4-((S)-4-acryloyl-3- (cyanomethyl)piperazin-1-yl)-7-(3- fluoro-2-(trifluoromethyl)phenyl)-2- (((S)-1-methylpyrrolidin-2- yl)methoxy)-5,6,7,8-tetrahydro-1,7- naphthyridine-3-carbonitrile; | 612 |
| 447 | 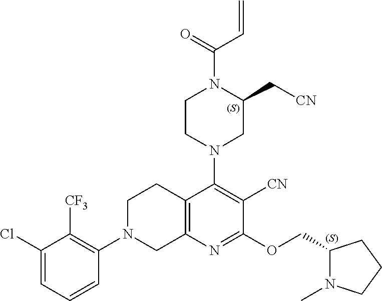 | 4-((S)-4-acryloyl-3- (cyanomethyl)piperazin-1-yl)-7-(3- chloro-2-(trifluoromethyl)phenyl)-2- (((S)-1-methylpyrrolidin-2- yl)methoxy)-5,6,7,8-tetrahydro-1,7- naphthyridine-3-carbonitrile; | 628 |
| 448 | 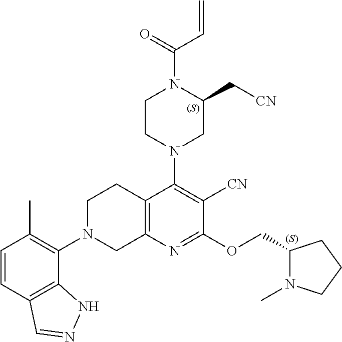 | 4-((S)-4-acryloyl-3- (cyanomethyl)piperazin-1-yl)-7-(6- methyl-hyl-1H-indazol-7-yl)-2-(((S)- 1-methylpyrrolidin-2-yl)methoxy)- 5,6,7,8-tetrahydro-1,7-naphthyridine- 3-carbonitrile; | 580 |
| 449 | 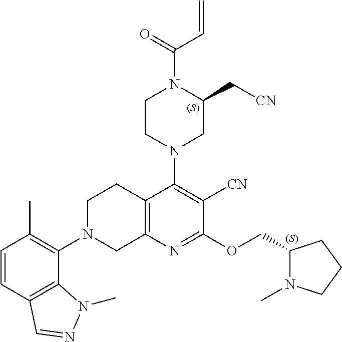 | 4-((S)-4-acryloyl-3- (cyanomethyl)piperazin-1-yl)-7-(1,6- dimethyl-1H-indazol-7-yl)-2-(((S)-1- methylpyrrolidin-2-yl)methoxy)- 5,6,7,8-tetrahydro-1,7-naphthyridine- 3-carbonitrile; | 594 |
| 450 | 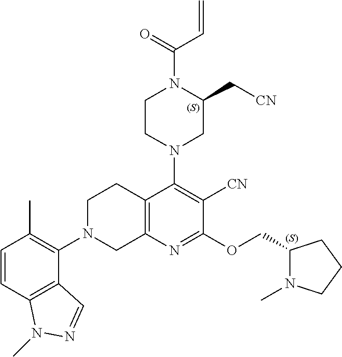 | 4-((S)-4-acryloyl-3- (cyanomethyl)piperazin-1-yl)-7-(1,5- dimethyl-1H-indazol-4-yl)-2-(((S)-1- methylpyrrolidin-2-yl)methoxy)- 5,6,7,8-tetrahydro-1,7-naphthyridine- 3-carbonitrile; | 594 |
| 451 | 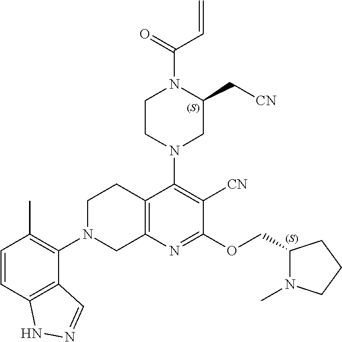 | 4-((S)-4-acryloyl-3- (cyanomethyl)piperazin-1-yl)-7-(5- methyl-1H-indazol-4-yl)-2-(((S)-1- methylpyrrolidin-2-yl)methoxy)- 5,6,7,8-tetrahydro-1,7-naphthyridine- 3-carbonitrile; | 580 |
| 452 | 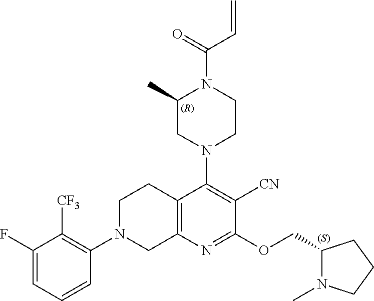 | 4-((R)-4-acryloyl-3-methylpiperazin- 1-yl)-7-(3-fluoro-2- (trifluoromethyl)phenyl)-2-(((S)-1- methylpyrrolidin-2-yl)methoxy)- 5,6,7,8-tetrahydro-1,7-naphthyridine- 3-carbonitrile; | 587 |
| 453 | 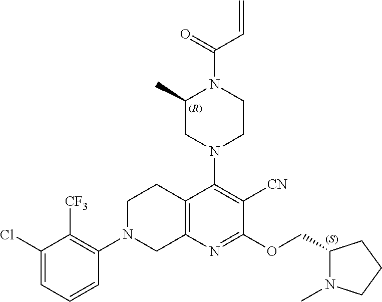 | 4-((R)-4-acryloyl-3-methylpiperazin- 1-yl)-7-(3-chloro-2- (trifluoromethyl)phenyl)-2-(((S)-1- methylpyrrolidin-2-yl)methoxy)- 5,6,7,8-tetrahydro-1,7-naphthyridine- 3-carbonitrile; | 603 |
| 454 | 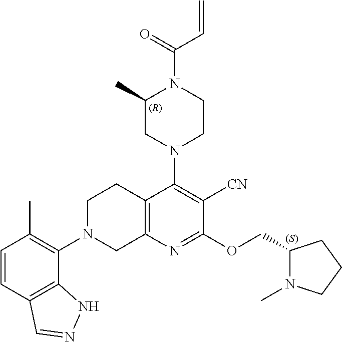 | 4-((R)-4-acryloyl-3-methylpiperazin- 1-yl)-7-(6-methyl-1H-indazol-7-yl)-2- (((S)-1-methylpyrrolidin-2- yl)methoxy)-5,6,7,8-tetrahydro-1,7- naphthyridine-3-carbonitrile; | 555 |
| 455 | 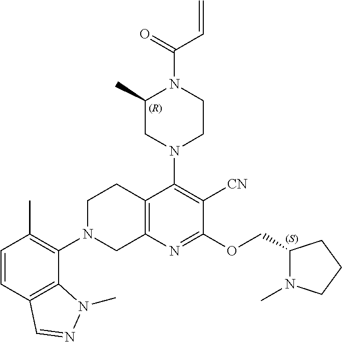 | 4-((R)-4-acryloyl-3-methylpiperazin- 1-yl)-7-(1,6-dimethyl-1H-indazol-7- yl)-2-(((S)-1-methylpyrrolidin-2- yl)methoxy)-5,6,7,8-tetrahydro-1,7- naphthyridine-3-carbonitrile; | 569 |
| 456 | 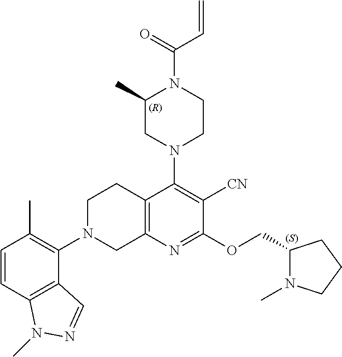 | 4-((R)-4-acryloyl-3-methylpiperazin- 1-yl)-7-(1,5-dimethyl-1H-indazol-4- yl)-2-(((S)-1-methylpyrrolidin-2- yl)methoxy)-5,6,7,8-tetrahydro-1,7- naphthyridine-3-carbonitrile; | 569 |
| 457 | 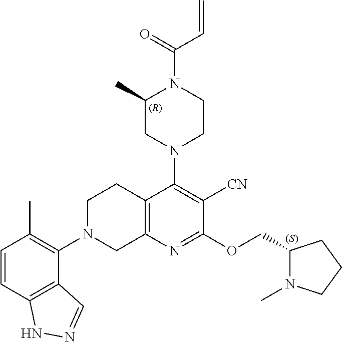 | 4-((R)-4-acryloyl-3-methylpiperazin- 1-yl)-7-(5-methyl-1H-indazol-4-yl)-2- (((S)-1-methylpyrrolidin-2- yl)methoxy)-5,6,7,8-tetrahydro-1,7- naphthyridine-3-carbonitrile; | 555 |
| 458 | 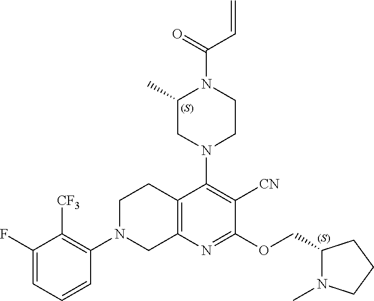 | 4-((S)-4-acryloyl-3-methylpiperazin- 1-yl)-7-(3-fluoro-2- (trifluoromethyl)phenyl)-2-(((S)-1- methylpyrrolidin-2-yl)methoxy)- 5,6,7,8-tetrahydro-1,7-naphthyridine- 3-carbonitrile; | 587 |
| 459 | 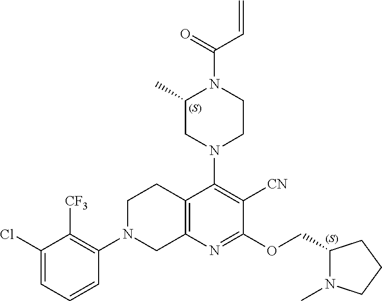 | 4-((S)-4-acryloyl-3-methylpiperazin- 1-yl)-7-(3-chloro-2- (trifluoromethyl)phenyl)-2-(((S)-1- methylpyrrolidin-2-yl)methoxy)- 5,6,7,8-tetrahydro-1,7-naphthyridine- 3-carbonitrile; | 603 |
| 460 | 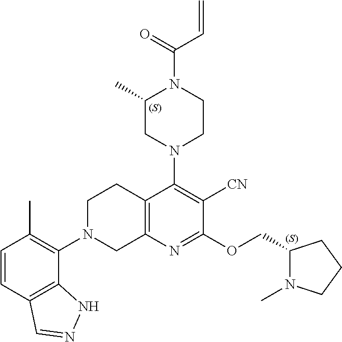 | 4-((S)-4-acryloyl-3-methylpiperazin- 1-yl)-7-(6-methyl-1H-indazol-7-yl)-2- (((S)-1-methylpyrrolidin-2- yl)methoxy)-5,6,7,8-tetrahydro-1,7- naphthyridine-3-carbonitrile; | 555 |
| 461 | 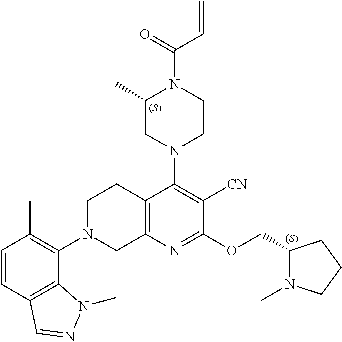 | 4-((S)-4-acryloyl-3-methylpiperazin- 1-yl)-7-(1,6-dimethyl-1H-indazol-7- yl)-2-(((S)-1-methylpyrrolidin-2- yl)methoxy)-5,6,7,8-tetrahydro-1,7- naphthyridine-3-carbonitrile; | 569 |
| 462 | 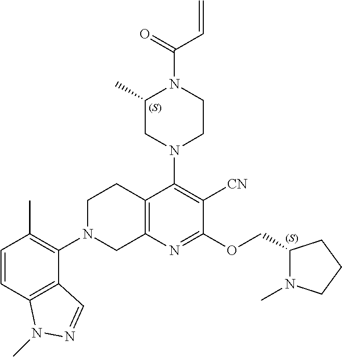 | 4-((S)-4-acryloyl-3-methylpiperazin- 1-yl)-7-(1,5-dimethyl-1H-indazol-4- yl)-2-(((S)-1-methylpyrrolidin-2- yl)methoxy)-5,6,7,8-tetrahydro-1,7- naphthyridine-3-carbonitrile; or | 569 |
| 463 | 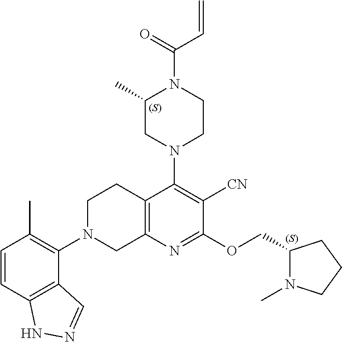 | 4-((S)-4-acryloyl-3-methylpiperazin- 1-yl)-7-(5-methyl-1H-indazol-4-yl)-2- (((S)-1-methylpyrrolidin-2- yl)methoxy)-5,6,7,8-tetrahydro-1,7- naphthyridine-3-carbonitrile. | 555 |

### PHARMACOLOGICAL TESTING

### 1. SOS1 Catalyzed Nucleotide Exchange Assay

HIS-KRAS (G12C, aa 2-185, Sino biological) was diluted to 5 μM in EDTA buffer (20 mM HEPES, pH 7.4, 50 mM NaCl, 10 mM EDTA, 0.01% (v/v) Tween-20) and incubated for 30 min at 25° C. The EDTA pretreated HIS-KRAS (G12C) was diluted to 12 nM in assay buffer (25 mM HEPES, pH 7.4, 120 mM NaCl, 5 mM MgCl₂, 1 mM DTT, 0.01% (v/v) Tween 20, 0.1% (w/v) BSA) containing 120 nM GDP (Sigma) and MAb Anti 6HIS-Tb cryptate Gold (Cisbio) and incubated for 1 hour at 25° C. to prepare GDP-loaded HIS-KRAS (G12C). The GDP-loaded HIS-KRAS (G12C) was pre-incubation with diluted compounds in a 384-well plate (Greiner) for 1 hour, then purified SOS1 ExD (Flag tag, aa 564-1049) and BODIPY™ FL GTP (Invitrogen) were added to the assay wells (Final concentration: 3 nM HIS-KRAS (G12C), 2 μM SOS1 ExD, 80 nM BODIPY™ FL GTP, 21 ng/mL MAb Anti 6HIS-Tb cryptate Gold) and incubated for 4 hours at 25° C. TR-FRET signals were then read on Tecan Spark multimode microplate reader. The parameters were F486: Excitation 340 (35) nm, Emission 486 (10) nm, Lag time 100 las, Integration time 200 las; F515: Excitation 340 (35) nm, Emission 515 (10) nm, Lag time 100 las, Integration time 200 las. TR-FRET ratios for each individual wells were calculated by equation: TR-FRET ratio=(Signal F515/Signal F486)*10000. Then the data were analyzed using a 4-parameter logistic model to calculate IC₅₀ values. The results of the SOS1 catalyzed nucleotide exchange assay are in the following Table:

| | SOS1 catalyzed nucleotide |
|---|---|
| Compound | exchange IC₅₀(nM) |
| Compound 1 | 53.07 |
| Compound 2 | >1000 |
| Compound 3 | >1000 |
| Compound 4 | >1000 |
| Compound 5 | >1000 |
| Compound 6 | >1000 |

It can be seen from the Table that the representative compound of the present invention had inhibitory activity in the SOS1 catalyzed nucleotide exchange assay.

What is claimed is:

1. A compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof, wherein, the compound is selected from:

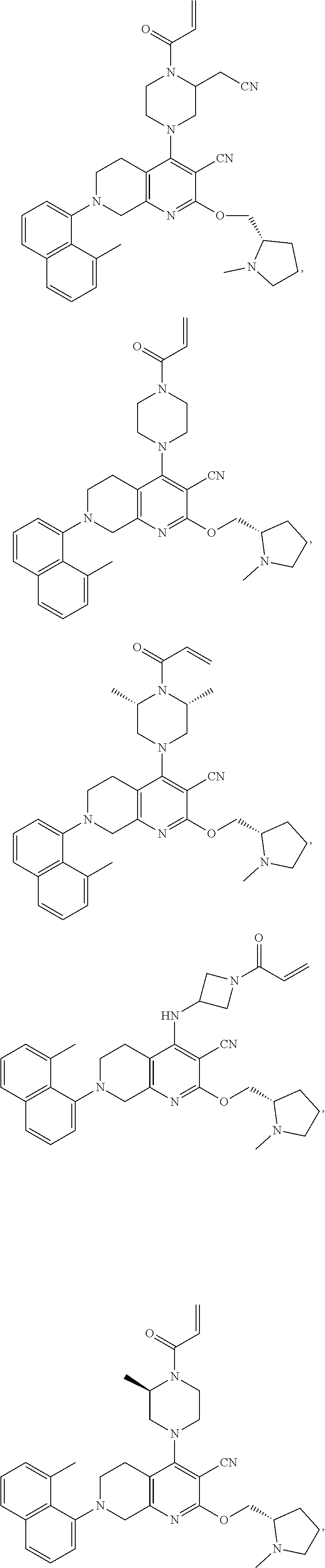 [SMILES: C=CC(=O)N1CCN(c2c(C#N)c(OC[C@@H]3CCCN3C)nc3c2CCN(c2cccc4cccc(C)c24)C3)CC1CC#N ; C=CC(=O)N1CCN(c2c(C#N)c(OC[C@@H]3CCCN3C)nc3c2CCN(c2cccc4cccc(C)c24)C3)CC1 ; C=CC(=O)N1[C@H](C)CN(c2c(C#N)c(OC[C@@H]3CCCN3C)nc3c2CCN(c2cccc4cccc(C)c24)C3)C[C@@H]1C ; C=CC(=O)N1CC(Nc2c(C#N)c(OC[C@@H]3CCCN3C)nc3c2CCN(c2cccc4cccc(C)c24)C3)C1 ; C=CC(=O)N1CCN(c2c(C#N)c(OC[C@@H]3CCCN3C)nc3c2CCN(c2cccc4cccc(C)c24)C3)C[C@H]1C]

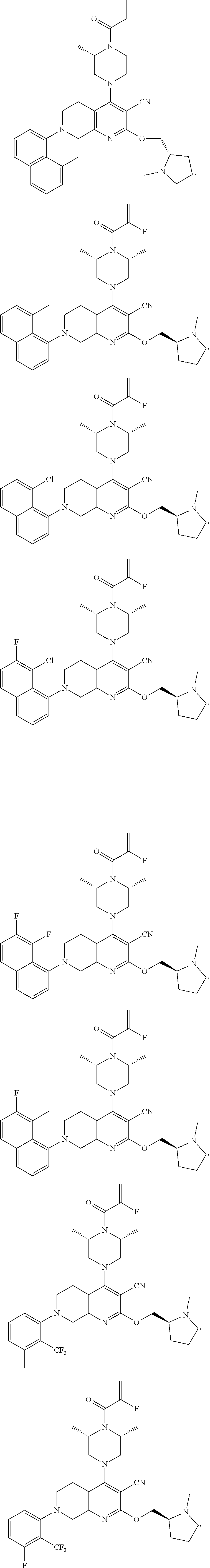

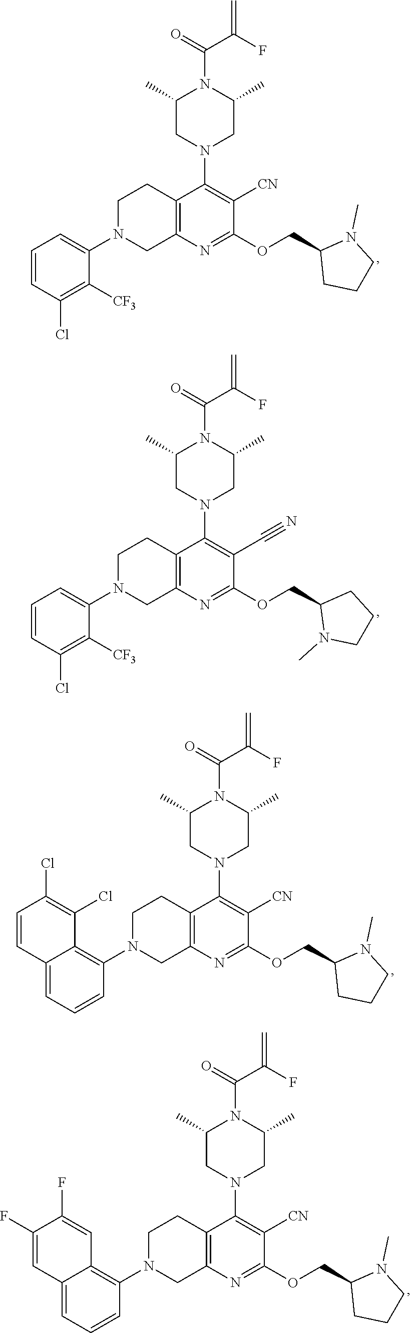 [SMILES: C=C(F)C(=O)N1[C@H](C)CN(c2c(C#N)c(OC[C@@H]3CCCN3C)nc3c2CCN(c2cccc(Cl)c2C(F)(F)F)C3)C[C@@H]1C ; C=C(F)C(=O)N1[C@H](C)CN(c2c(C#N)c(OC[C@H]3CCCN3C)nc3c2CCN(c2cccc(Cl)c2C(F)(F)F)C3)C[C@@H]1C ; C=C(F)C(=O)N1[C@H](C)CN(c2c(C#N)c(OC[C@@H]3CCCN3C)nc3c2CCN(c2cccc4ccc(Cl)c(Cl)c24)C3)C[C@@H]1C ; C=C(F)C(=O)N1[C@H](C)CN(c2c(C#N)c(OC[C@@H]3CCCN3C)nc3c2CCN(c2cccc4cc(F)c(F)cc24)C3)C[C@@H]1C]

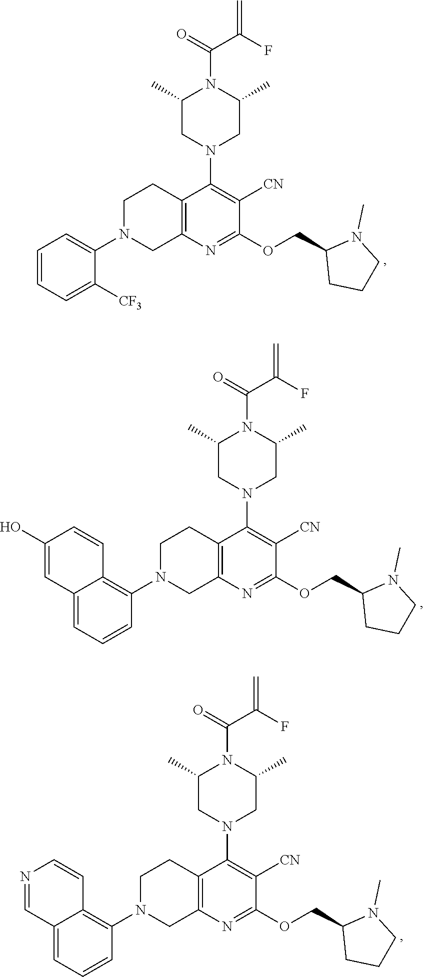 [SMILES: C=C(F)C(=O)N1[C@H](C)CN(c2c(C#N)c(OC[C@@H]3CCCN3C)nc3c2CCN(c2ccccc2C(F)(F)F)C3)C[C@@H]1C ; C=C(F)C(=O)N1[C@H](C)CN(c2c(C#N)c(OC[C@@H]3CCCN3C)nc3c2CCN(c2cccc4cc(O)ccc24)C3)C[C@@H]1C ; C=C(F)C(=O)N1[C@H](C)CN(c2c(C#N)c(OC[C@@H]3CCCN3C)nc3c2CCN(c2cccc4cnccc24)C3)C[C@@H]1C]

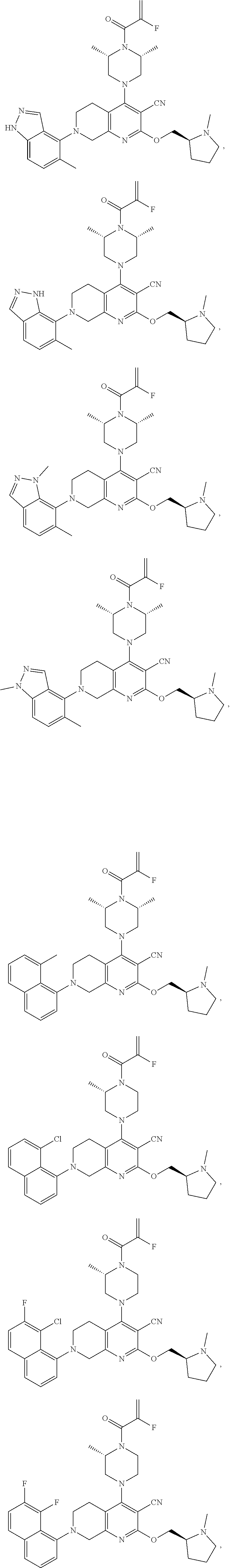

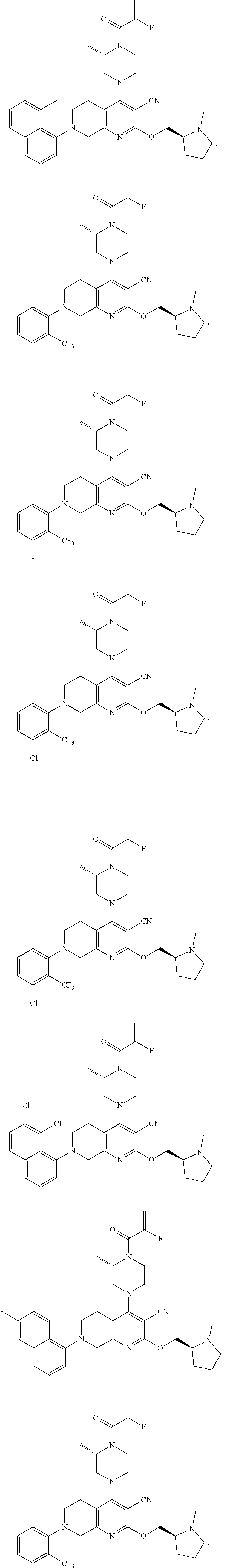

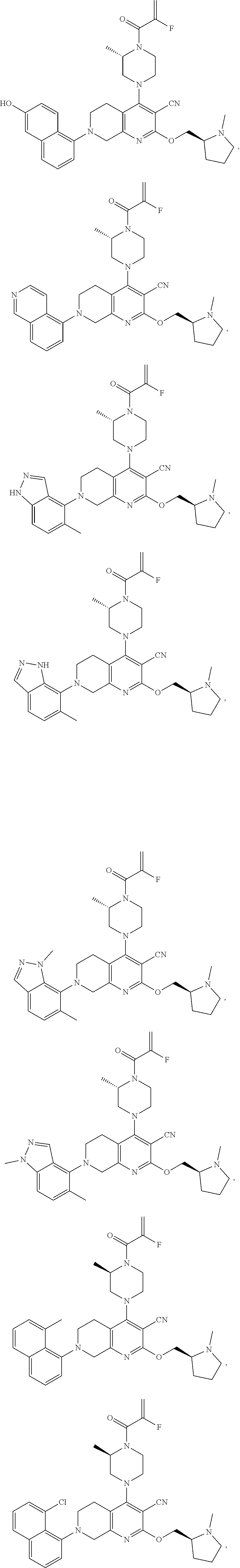

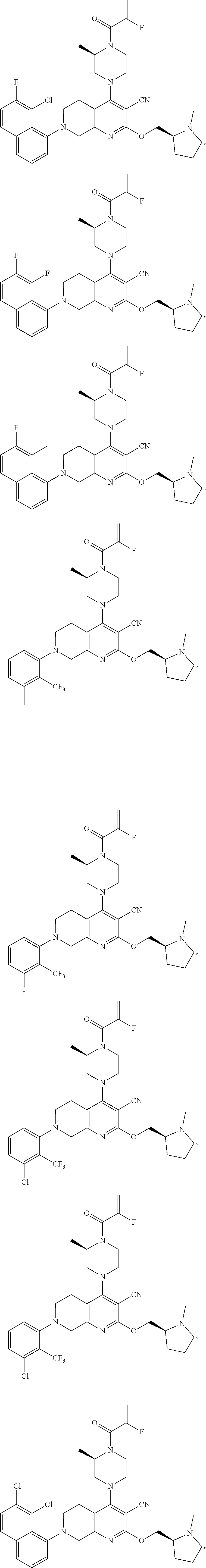

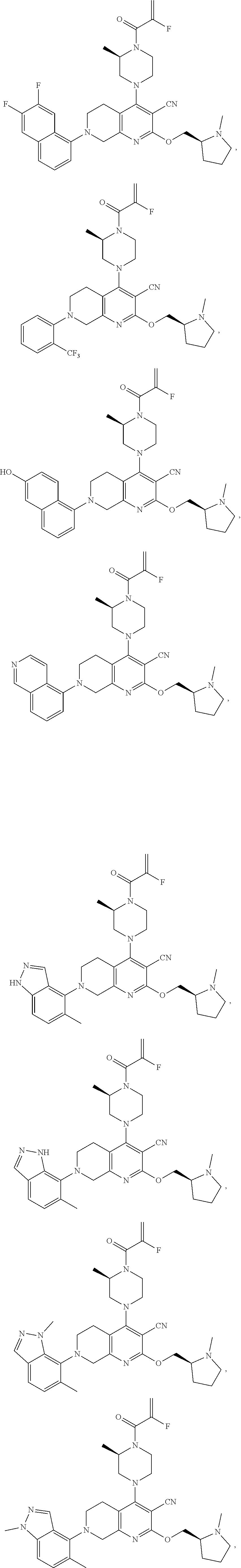

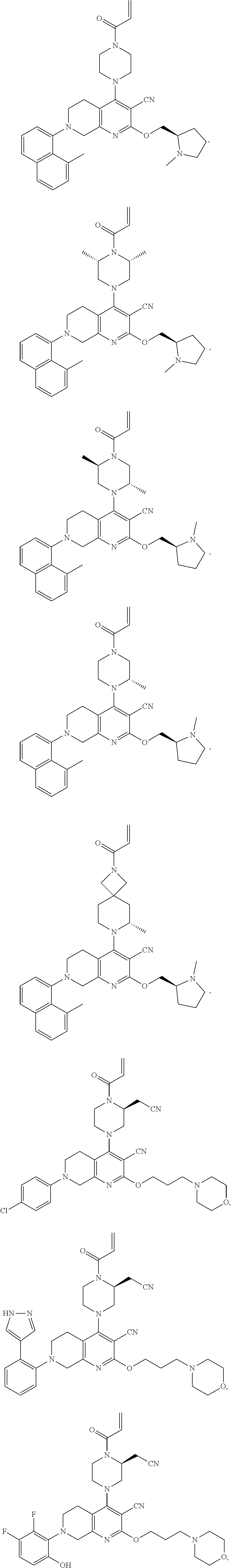

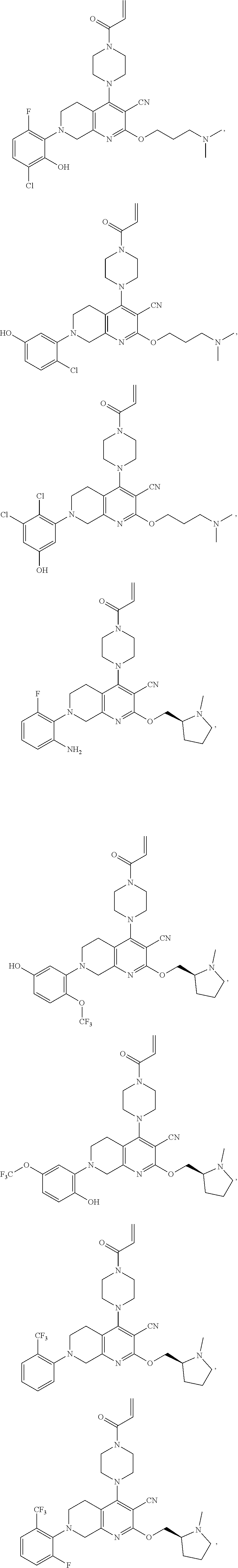

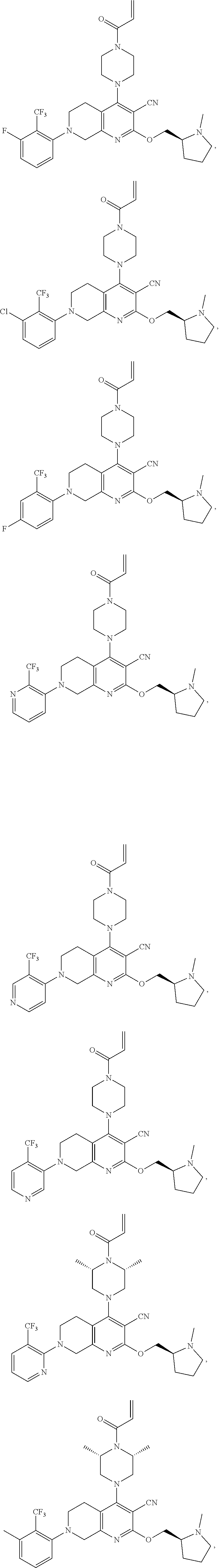

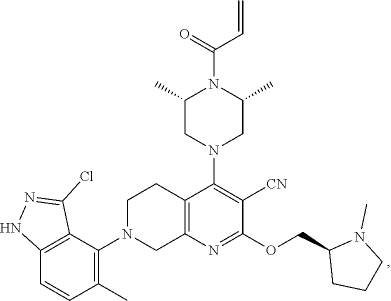 [SMILES: C=CC(=O)N1[C@H](C)CN(c2c(C#N)c(OC[C@@H]3CCCN3C)nc3c2CCN(c2c(C)ccc4[nH]nc(Cl)c24)C3)C[C@@H]1C]

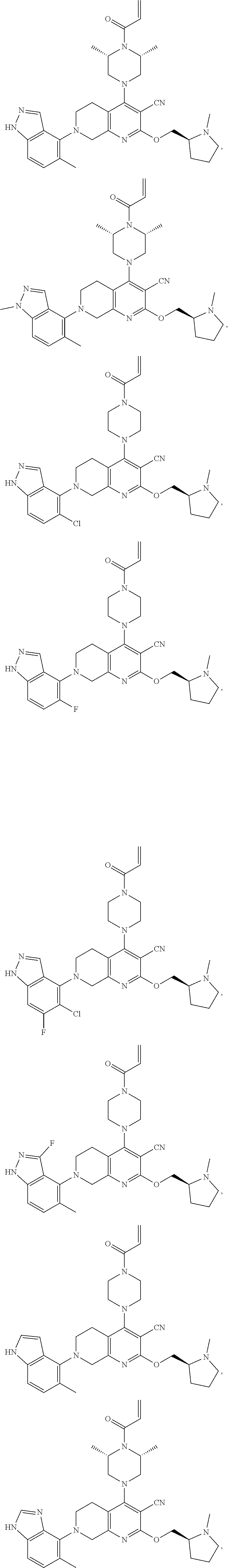

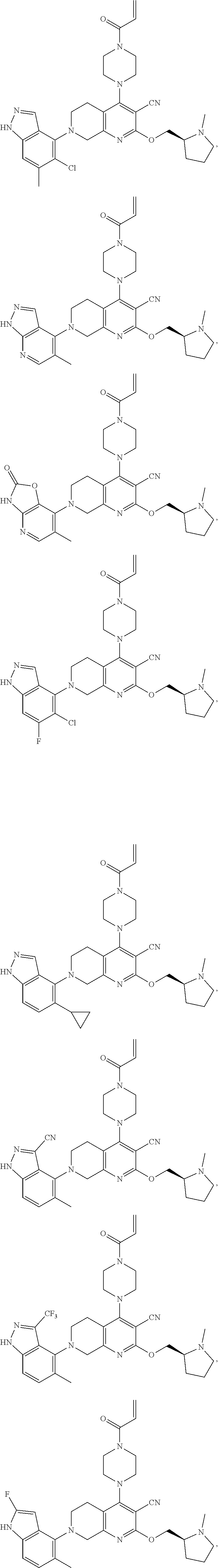

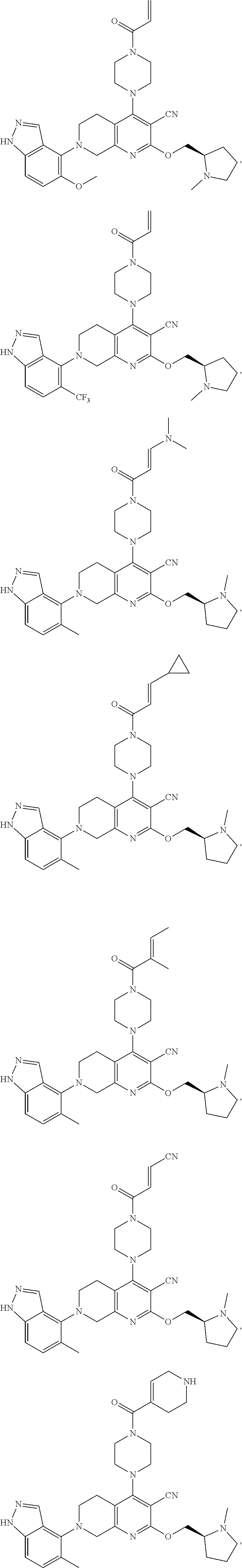

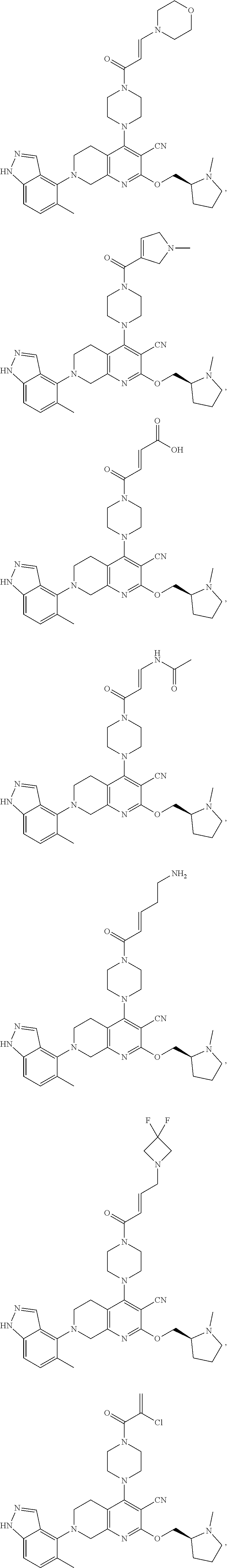

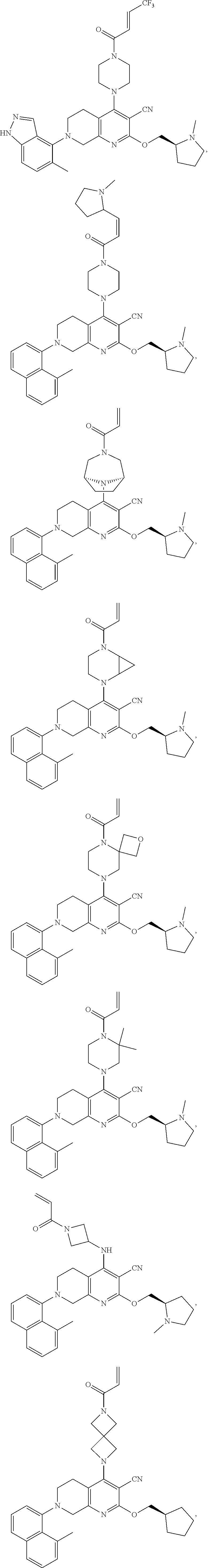

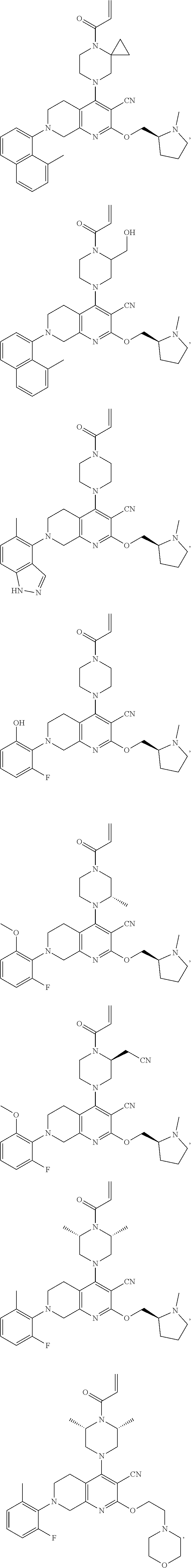

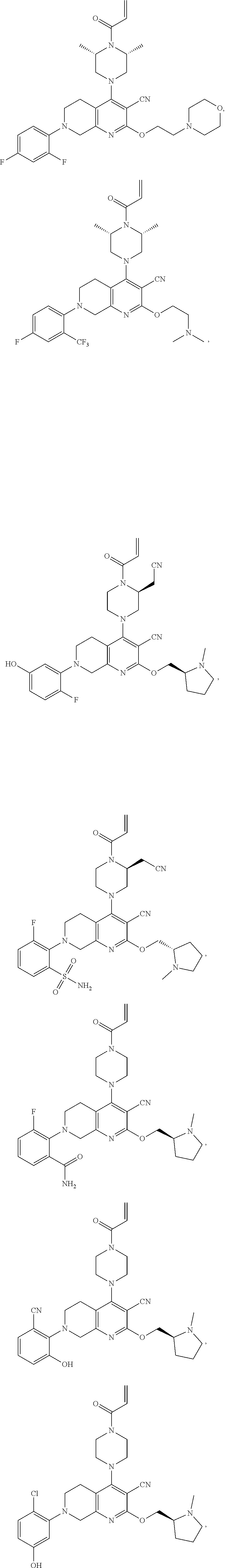

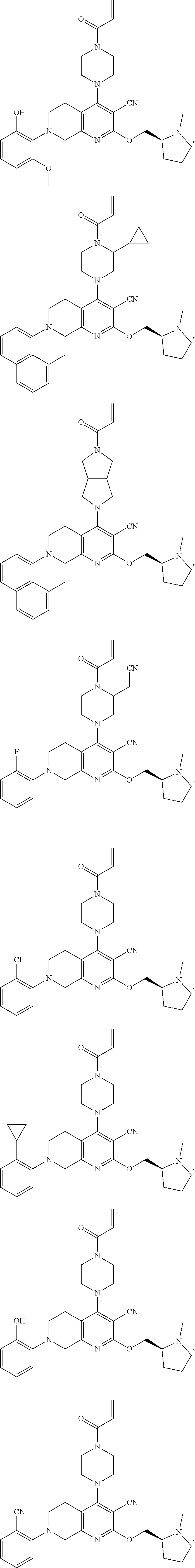

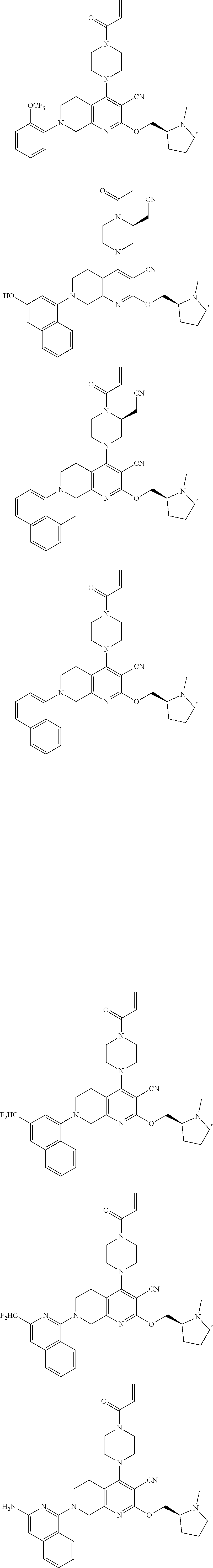

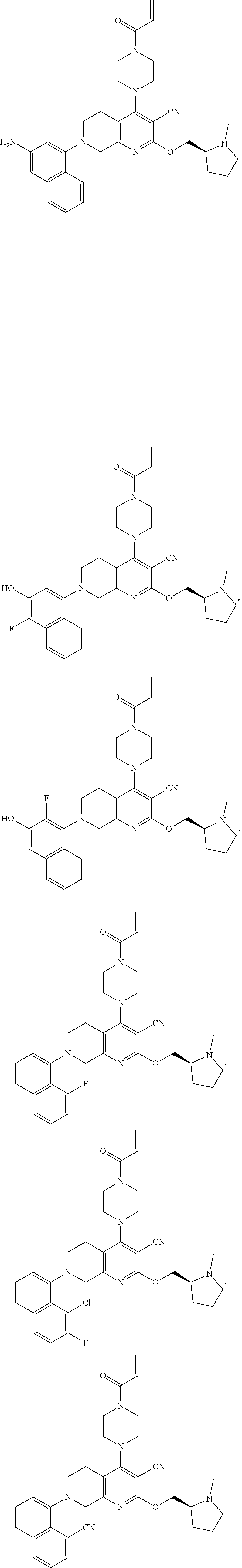 [SMILES: C=CC(=O)N1CCN(c2c(C#N)c(OC[C@@H]3CCCN3C)nc3c2CCN(c2cc(N)cc4ccccc24)C3)CC1 ; C=CC(=O)N1CCN(c2c(C#N)c(OC[C@@H]3CCCN3C)nc3c2CCN(c2cc(O)c(F)c4ccccc24)C3)CC1 ; C=CC(=O)N1CCN(c2c(C#N)c(OC[C@@H]3CCCN3C)nc3c2CCN(c2c(F)c(O)cc4ccccc24)C3)CC1 ; C=CC(=O)N1CCN(c2c(C#N)c(OC[C@@H]3CCCN3C)nc3c2CCN(c2cccc4cccc(F)c24)C3)CC1 ; C=CC(=O)N1CCN(c2c(C#N)c(OC[C@@H]3CCCN3C)nc3c2CCN(c2cccc4ccc(F)c(Cl)c24)C3)CC1 ; C=CC(=O)N1CCN(c2c(C#N)c(OC[C@@H]3CCCN3C)nc3c2CCN(c2cccc4cccc(C#N)c24)C3)CC1]

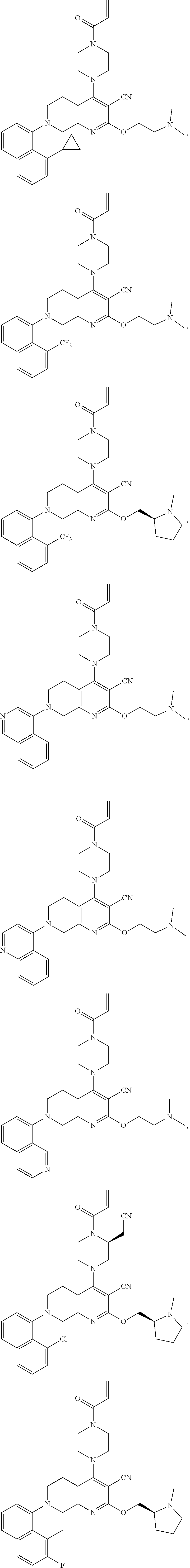

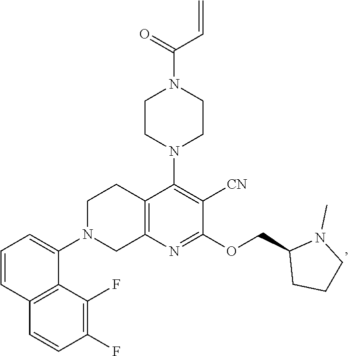 [SMILES: C=CC(=O)N1CCN(c2c(C#N)c(OC[C@@H]3CCCN3C)nc3c2CCN(c2cccc4ccc(F)c(F)c24)C3)CC1]

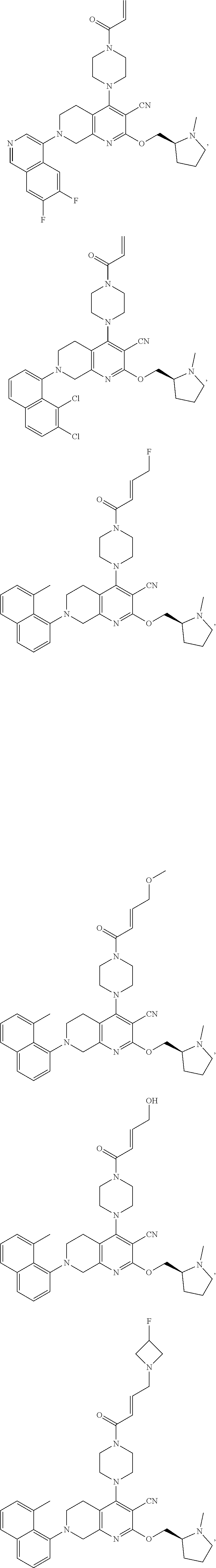 [SMILES: C=CC(=O)N1CCN(c2c(C#N)c(OC[C@@H]3CCCN3C)nc3c2CCN(c2cncc4cc(F)c(F)cc24)C3)CC1 ; C=CC(=O)N1CCN(c2c(C#N)c(OC[C@@H]3CCCN3C)nc3c2CCN(c2cccc4ccc(Cl)c(Cl)c24)C3)CC1 ; Cc1cccc2cccc(N3CCc4c(nc(OC[C@@H]5CCCN5C)c(C#N)c4N4CCN(C(=O)/C=C/CF)CC4)C3)c12 ; COC/C=C/C(=O)N1CCN(c2c(C#N)c(OC[C@@H]3CCCN3C)nc3c2CCN(c2cccc4cccc(C)c24)C3)CC1 ; Cc1cccc2cccc(N3CCc4c(nc(OC[C@@H]5CCCN5C)c(C#N)c4N4CCN(C(=O)/C=C/CO)CC4)C3)c12 ; Cc1cccc2cccc(N3CCc4c(nc(OC[C@@H]5CCCN5C)c(C#N)c4N4CCN(C(=O)/C=C/CN5CC(F)C5)CC4)C3)c12]

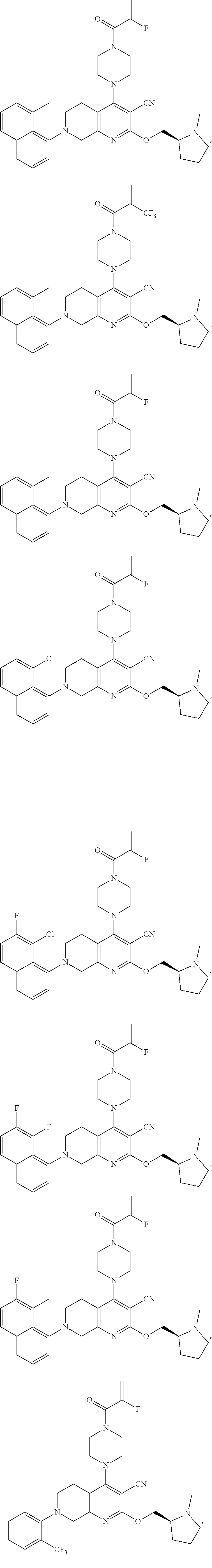

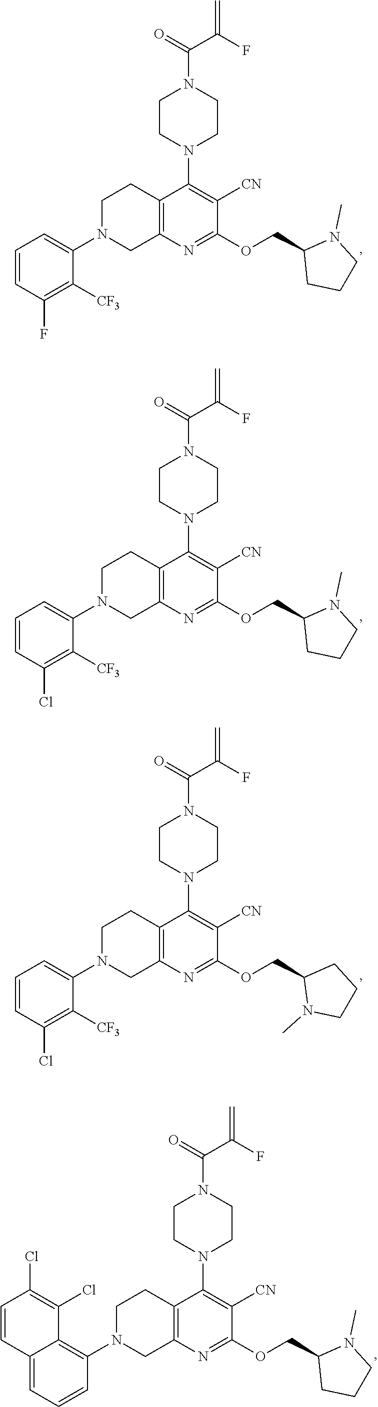 [SMILES: C=C(F)C(=O)N1CCN(c2c(C#N)c(OC[C@@H]3CCCN3C)nc3c2CCN(c2cccc(F)c2C(F)(F)F)C3)CC1 ; C=C(F)C(=O)N1CCN(c2c(C#N)c(OC[C@@H]3CCCN3C)nc3c2CCN(c2cccc(Cl)c2C(F)(F)F)C3)CC1 ; C=C(F)C(=O)N1CCN(c2c(C#N)c(OC[C@H]3CCCN3C)nc3c2CCN(c2cccc(Cl)c2C(F)(F)F)C3)CC1 ; C=C(F)C(=O)N1CCN(c2c(C#N)c(OC[C@@H]3CCCN3C)nc3c2CCN(c2cccc4ccc(Cl)c(Cl)c24)C3)CC1]

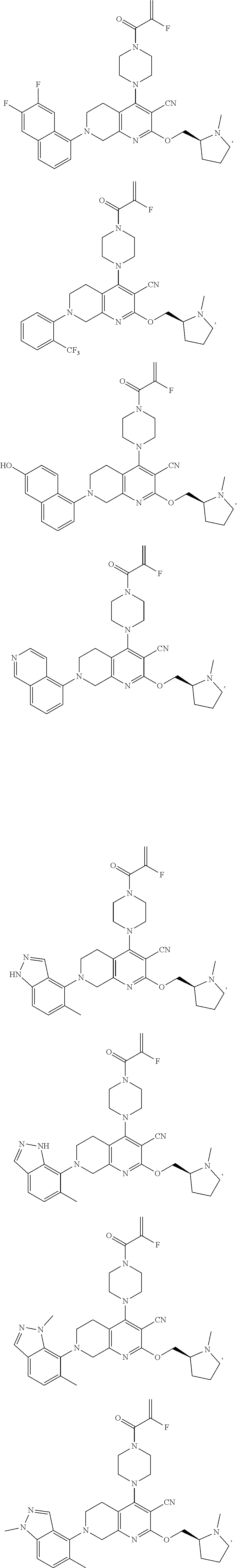

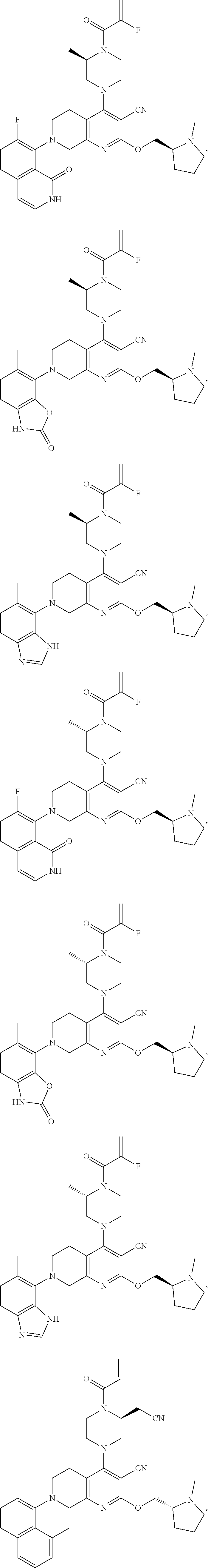

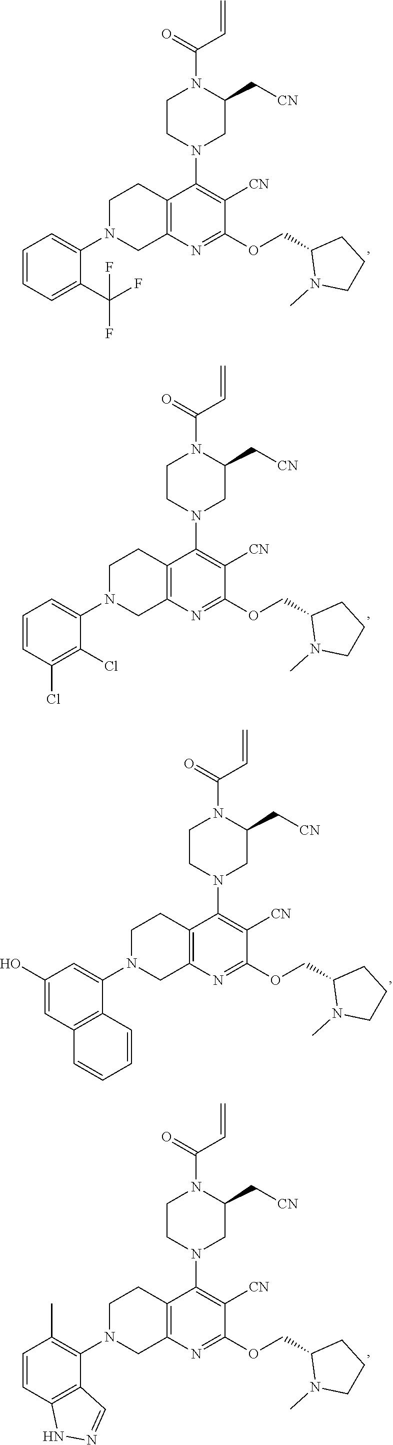 [SMILES: C=CC(=O)N1CCN(c2c(C#N)c(OC[C@@H]3CCCN3C)nc3c2CCN(c2ccccc2C(F)(F)F)C3)C[C@@H]1CC#N ; C=CC(=O)N1CCN(c2c(C#N)c(OC[C@@H]3CCCN3C)nc3c2CCN(c2cccc(Cl)c2Cl)C3)C[C@@H]1CC#N ; C=CC(=O)N1CCN(c2c(C#N)c(OC[C@@H]3CCCN3C)nc3c2CCN(c2cc(O)cc4ccccc24)C3)C[C@@H]1CC#N ; C=CC(=O)N1CCN(c2c(C#N)c(OC[C@@H]3CCCN3C)nc3c2CCN(c2c(C)ccc4[nH]ncc24)C3)C[C@@H]1CC#N]

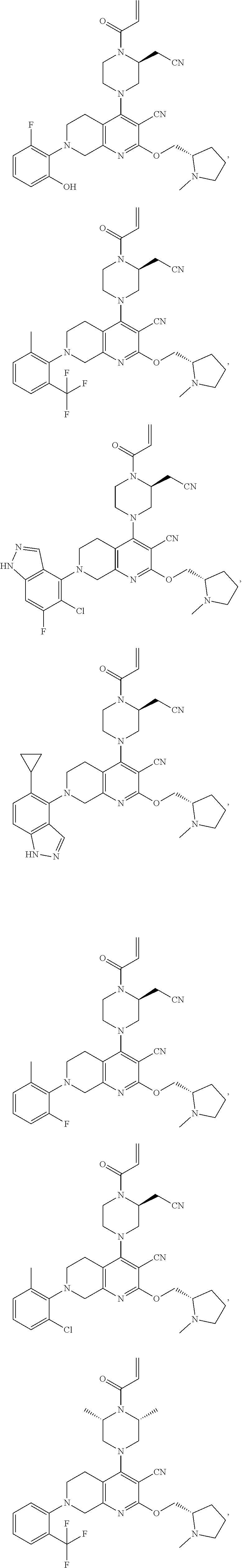

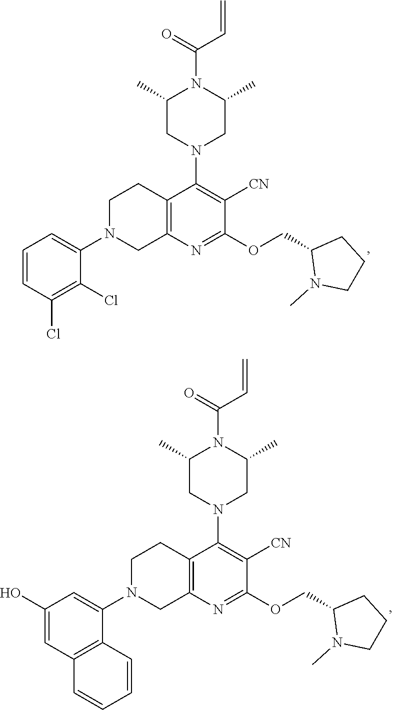 [SMILES: C=CC(=O)N1[C@H](C)CN(c2c(C#N)c(OC[C@@H]3CCCN3C)nc3c2CCN(c2cccc(Cl)c2Cl)C3)C[C@@H]1C ; C=CC(=O)N1[C@H](C)CN(c2c(C#N)c(OC[C@@H]3CCCN3C)nc3c2CCN(c2cc(O)cc4ccccc24)C3)C[C@@H]1C]

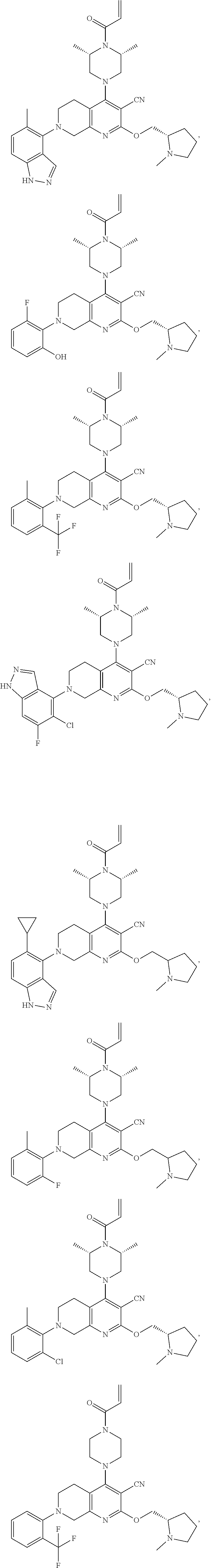

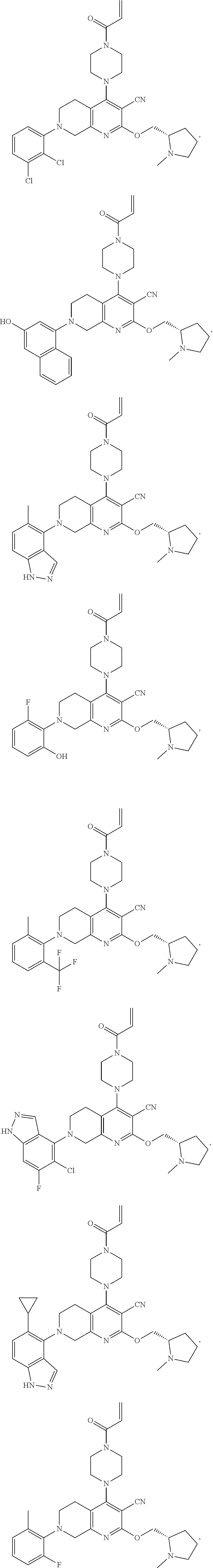

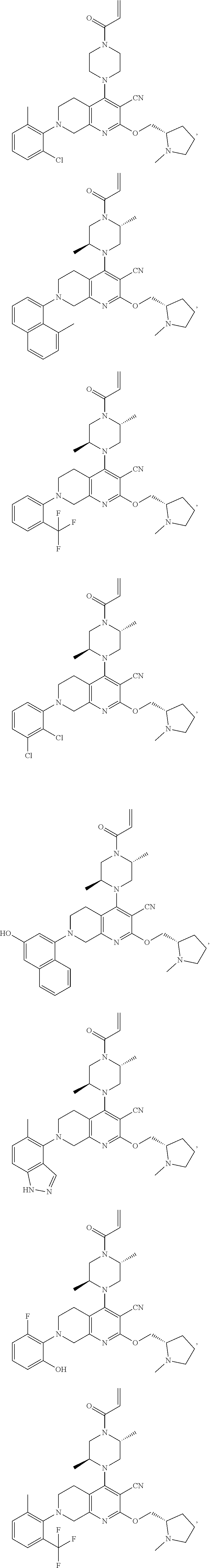

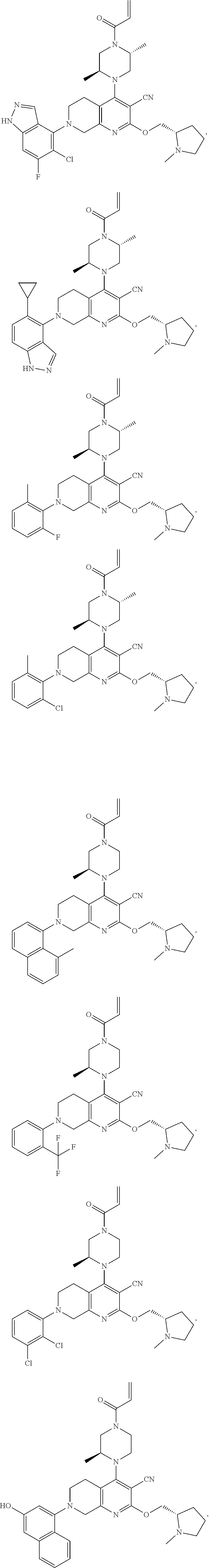

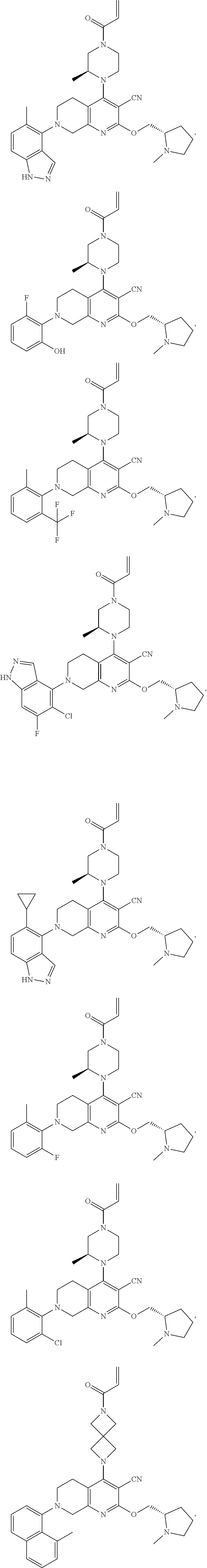

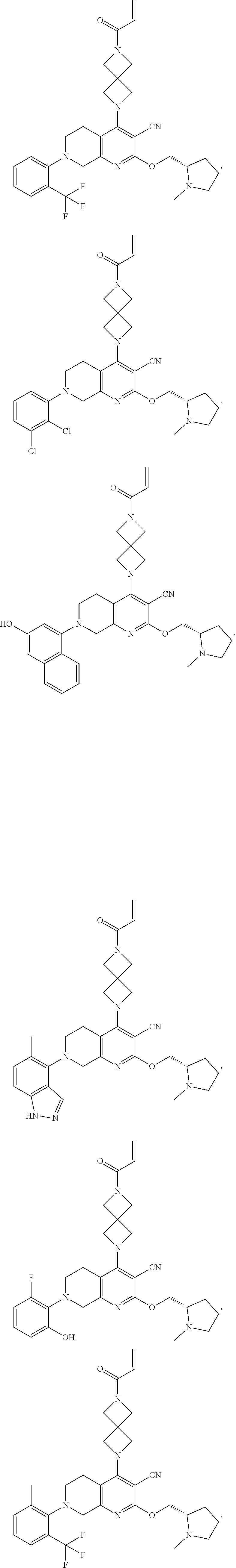 [SMILES: C=CC(=O)N1CC2(C1)CN(c1c(C#N)c(OC[C@@H]3CCCN3C)nc3c1CCN(c1ccccc1C(F)(F)F)C3)C2 ; C=CC(=O)N1CC2(C1)CN(c1c(C#N)c(OC[C@@H]3CCCN3C)nc3c1CCN(c1cccc(Cl)c1Cl)C3)C2 ; C=CC(=O)N1CC2(C1)CN(c1c(C#N)c(OC[C@@H]3CCCN3C)nc3c1CCN(c1cc(O)cc4ccccc14)C3)C2 ; C=CC(=O)N1CC2(C1)CN(c1c(C#N)c(OC[C@@H]3CCCN3C)nc3c1CCN(c1c(C)ccc4[nH]ncc14)C3)C2 ; C=CC(=O)N1CC2(C1)CN(c1c(C#N)c(OC[C@@H]3CCCN3C)nc3c1CCN(c1c(O)cccc1F)C3)C2 ; C=CC(=O)N1CC2(C1)CN(c1c(C#N)c(OC[C@@H]3CCCN3C)nc3c1CCN(c1c(C)cccc1C(F)(F)F)C3)C2]

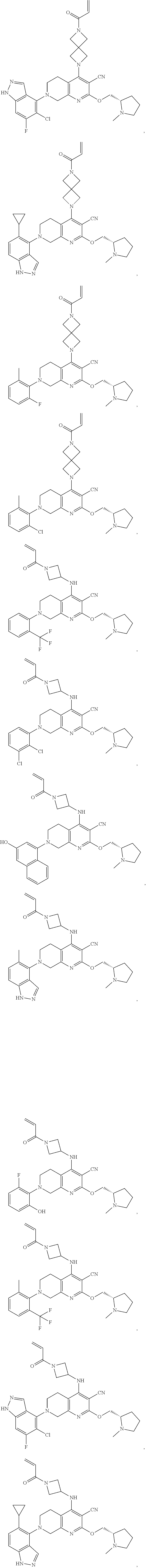

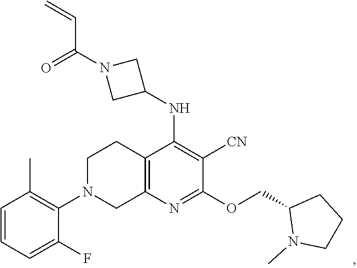 [SMILES: C=CC(=O)N1CC(Nc2c(C#N)c(OC[C@@H]3CCCN3C)nc3c2CCN(c2c(C)cccc2F)C3)C1]

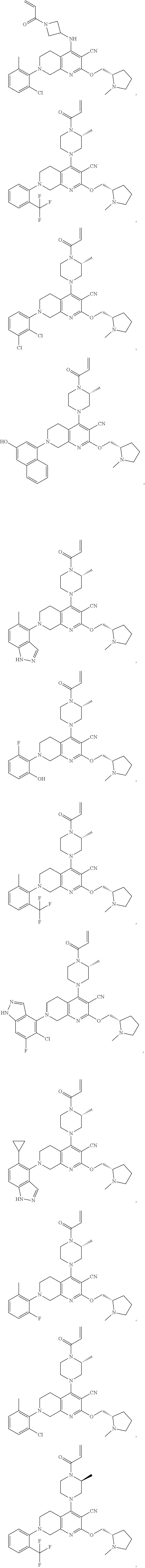

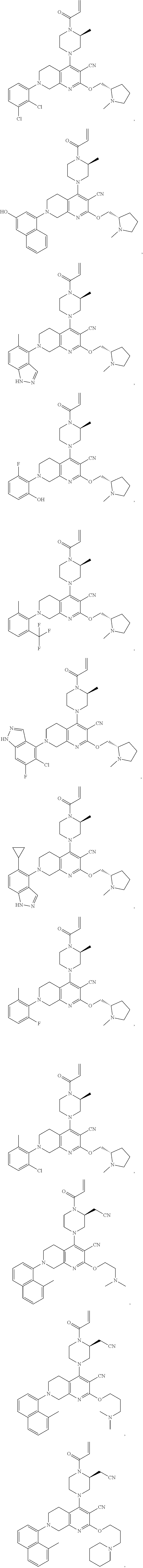

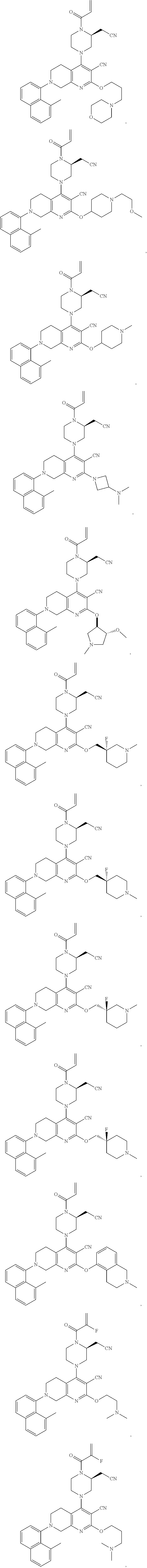

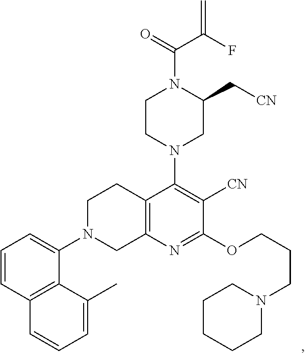 [SMILES: C=C(F)C(=O)N1CCN(c2c(C#N)c(OCCCN3CCCCC3)nc3c2CCN(c2cccc4cccc(C)c24)C3)C[C@@H]1CC#N]

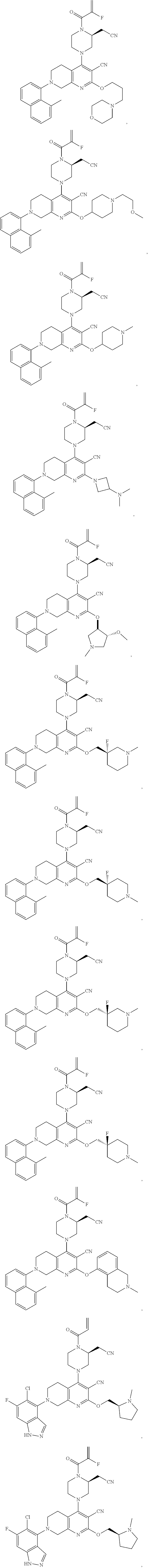

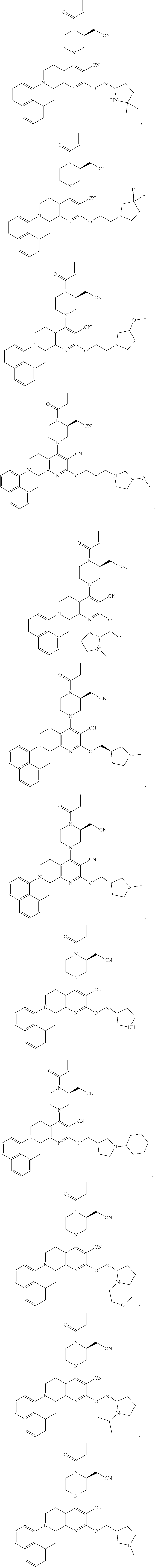

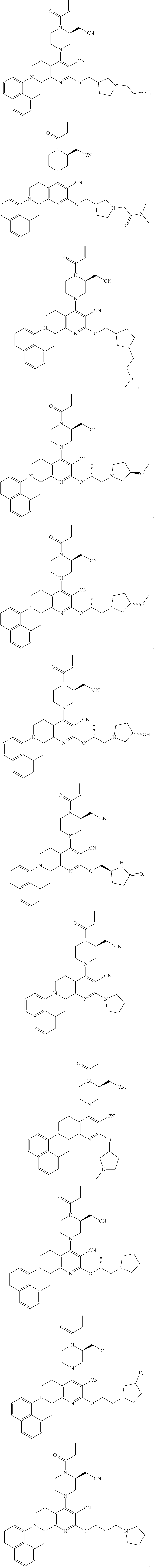

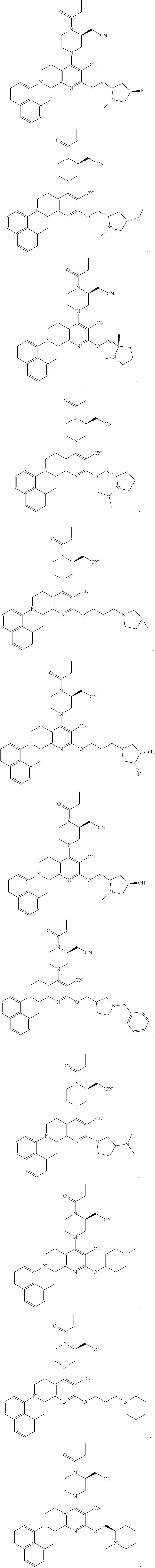

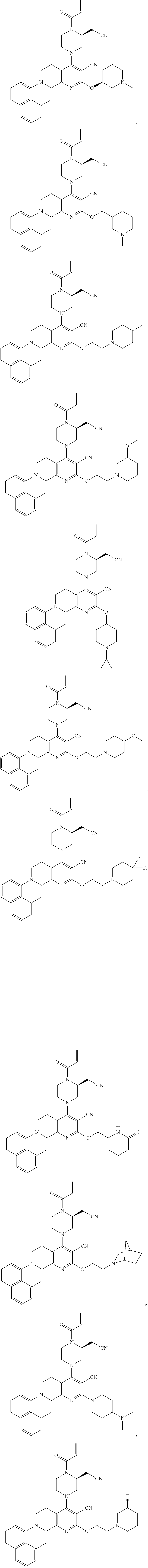

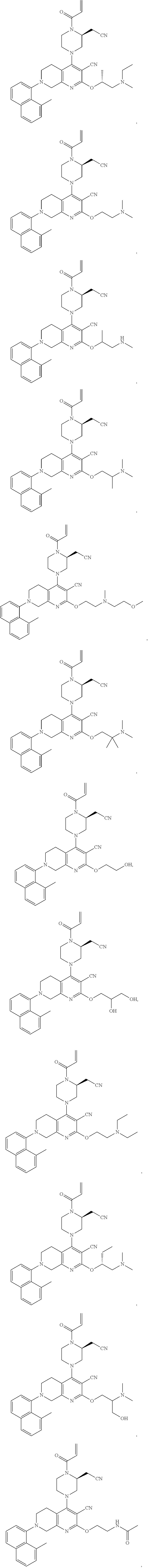

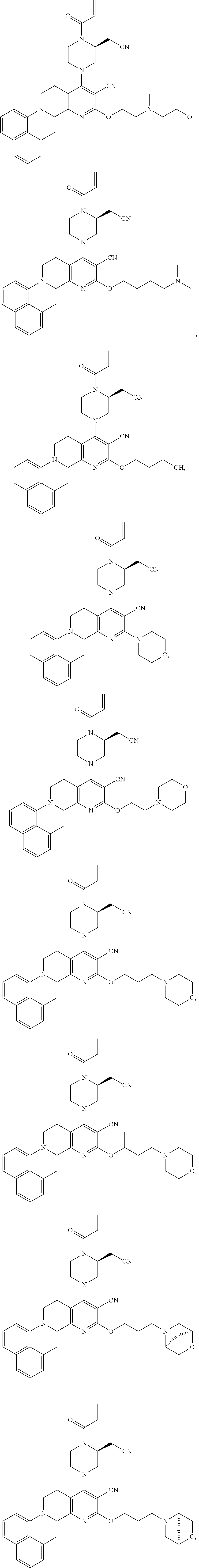

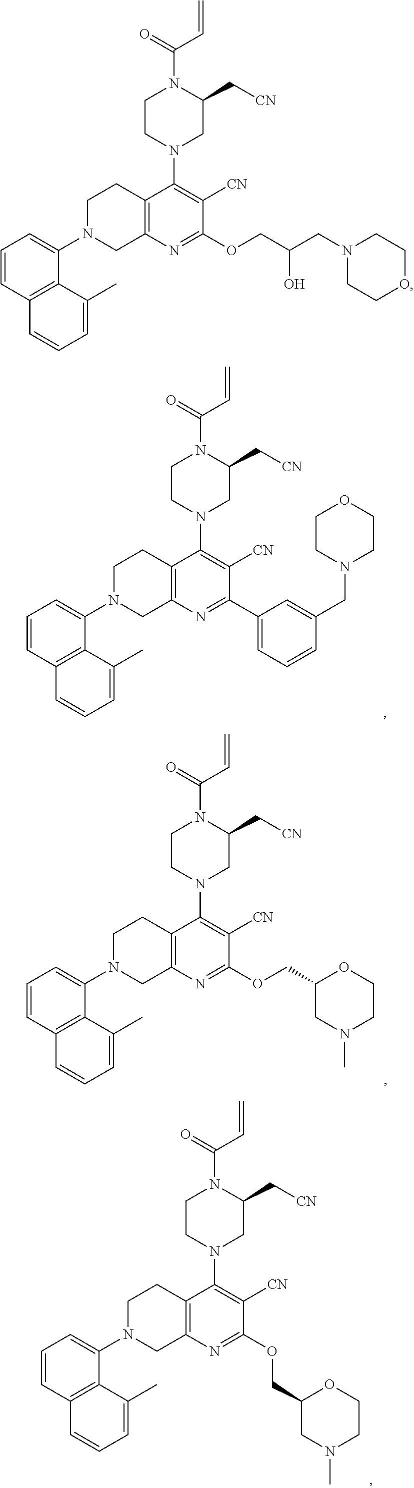 [SMILES: C=CC(=O)N1CCN(c2c(C#N)c(OCC(O)CN3CCOCC3)nc3c2CCN(c2cccc4cccc(C)c24)C3)C[C@@H]1CC#N ; C=CC(=O)N1CCN(c2c(C#N)c(-c3cccc(CN4CCOCC4)c3)nc3c2CCN(c2cccc4cccc(C)c24)C3)C[C@@H]1CC#N ; C=CC(=O)N1CCN(c2c(C#N)c(OC[C@H]3CN(C)CCO3)nc3c2CCN(c2cccc4cccc(C)c24)C3)C[C@@H]1CC#N ; C=CC(=O)N1CCN(c2c(C#N)c(OC[C@@H]3CN(C)CCO3)nc3c2CCN(c2cccc4cccc(C)c24)C3)C[C@@H]1CC#N]

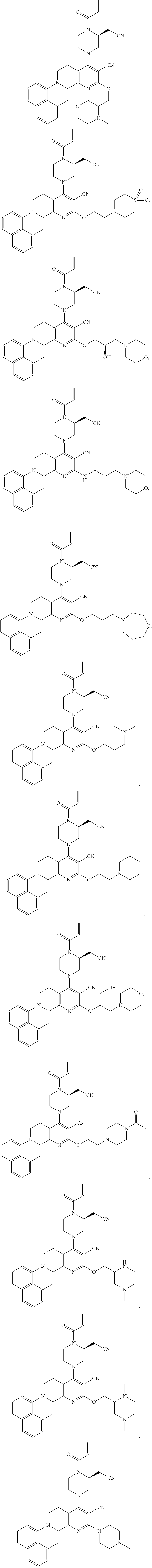

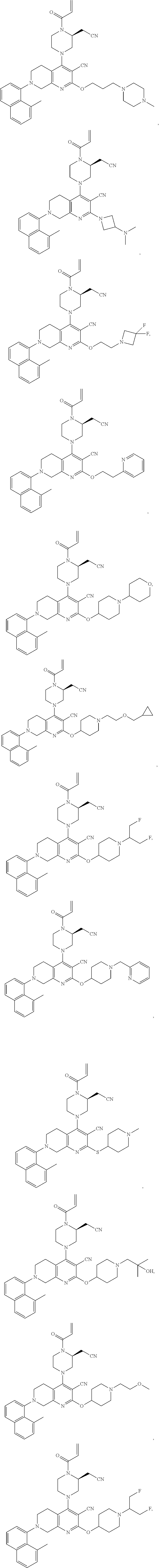

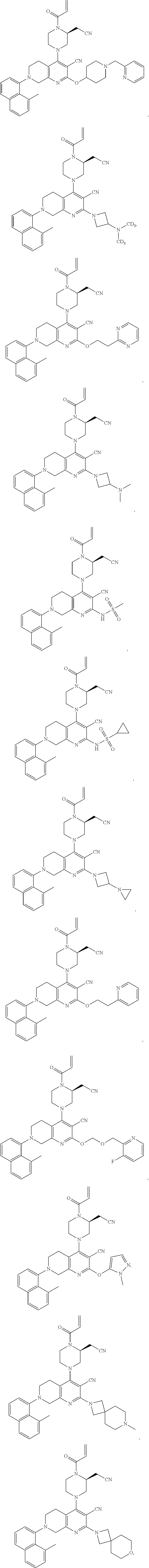

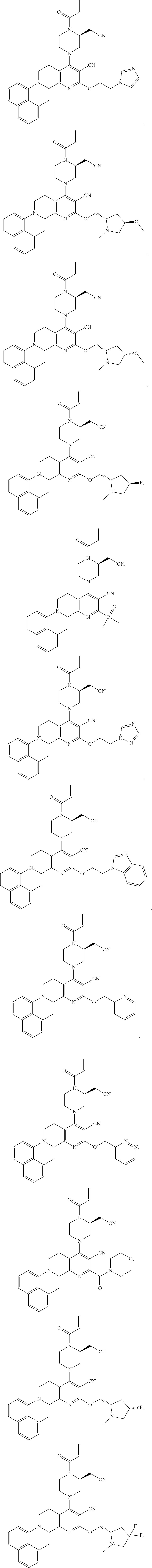

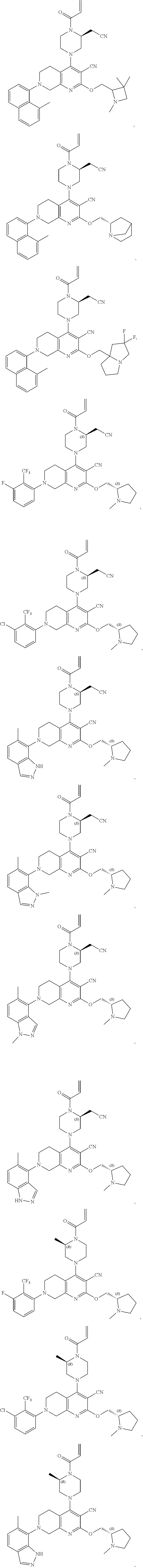

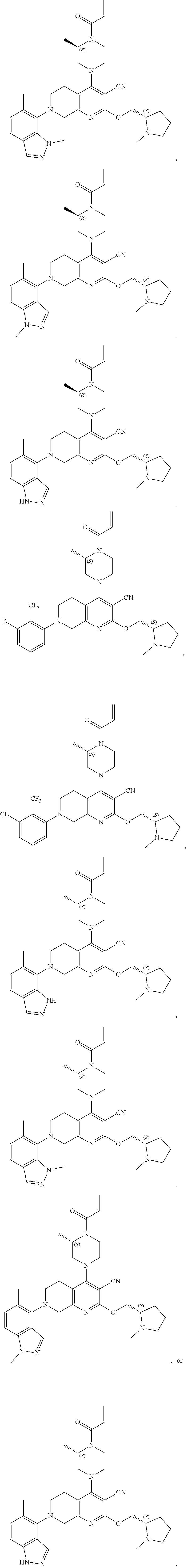

2. The compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, wherein, the compound is:

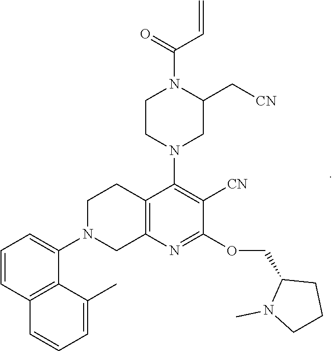 [SMILES: C=CC(=O)N1CCN(c2c(C#N)c(OC[C@@H]3CCCN3C)nc3c2CCN(c2cccc4cccc(C)c24)C3)CC1CC#N ; "."]

3. The compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, wherein, the compound is:

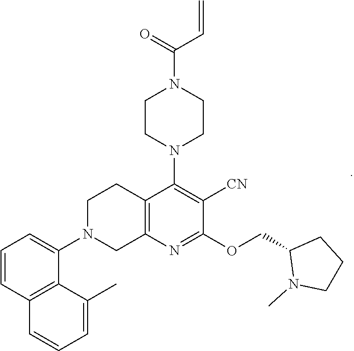 [SMILES: C=CC(=O)N1CCN(c2c(C#N)c(OC[C@@H]3CCCN3C)nc3c2CCN(c2cccc4cccc(C)c24)C3)CC1 ; "."]

4. The compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, wherein, the compound is:

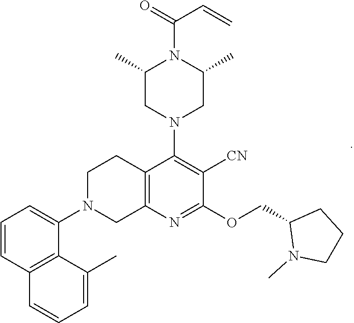 [SMILES: C=CC(=O)N1[C@H](C)CN(c2c(C#N)c(OC[C@@H]3CCCN3C)nc3c2CCN(c2cccc4cccc(C)c24)C3)C[C@@H]1C ; "."]

5. The compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, wherein, the compound is:

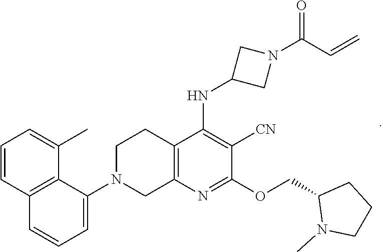 [SMILES: C=CC(=O)N1CC(Nc2c(C#N)c(OC[C@@H]3CCCN3C)nc3c2CCN(c2cccc4cccc(C)c24)C3)C1 ; "."]

6. The compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, wherein, the compound is:

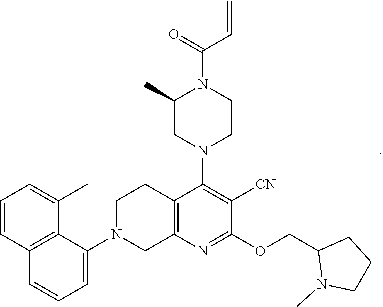 [SMILES: C=CC(=O)N1CCN(c2c(C#N)c(OCC3CCCN3C)nc3c2CCN(c2cccc4cccc(C)c24)C3)C[C@H]1C ; "."]

7. A pharmaceutical composition comprising at least one compound, a stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1, and at least one pharmaceutically acceptable excipient.

8. The pharmaceutical composition according to claim 7, wherein the compound is:

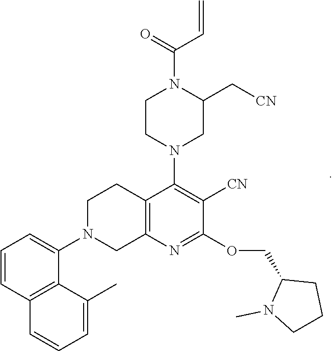 [SMILES: C=CC(=O)N1CCN(c2c(C#N)c(OC[C@@H]3CCCN3C)nc3c2CCN(c2cccc4cccc(C)c24)C3)CC1CC#N ; "."]

9. The pharmaceutical composition according to claim 7, wherein the compound is:

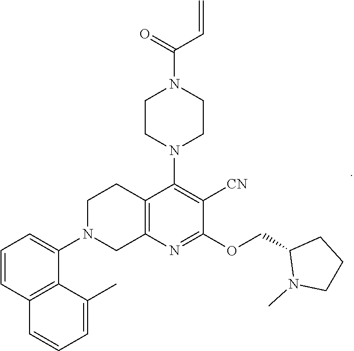 [SMILES: C=CC(=O)N1CCN(c2c(C#N)c(OC[C@@H]3CCCN3C)nc3c2CCN(c2cccc4cccc(C)c24)C3)CC1 ; "."]

10. The pharmaceutical composition according to claim 7, wherein the compound is:

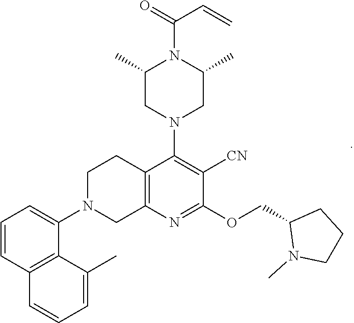 [SMILES: C=CC(=O)N1[C@H](C)CN(c2c(C#N)c(OC[C@@H]3CCCN3C)nc3c2CCN(c2cccc4cccc(C)c24)C3)C[C@@H]1C ; "."]

11. The pharmaceutical composition according to claim 7, wherein the compound is:

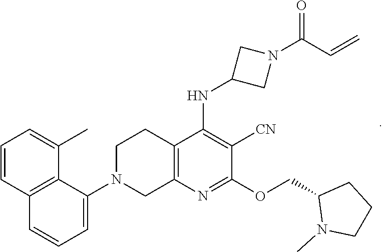 [SMILES: C=CC(=O)N1CC(Nc2c(C#N)c(OC[C@@H]3CCCN3C)nc3c2CCN(c2cccc4cccc(C)c24)C3)C1 ; "."]

12. The pharmaceutical composition according to claim 7, wherein the compound is:

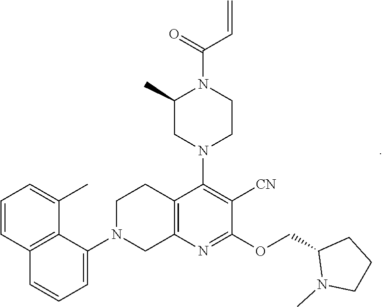 [SMILES: C=CC(=O)N1CCN(c2c(C#N)c(OC[C@@H]3CCCN3C)nc3c2CCN(c2cccc4cccc(C)c24)C3)C[C@H]1C ; "."]

13. A method of treating a subject having a cancer related to KRAS G12C mutant protein, said method comprising administering to the subject a therapeutically effective amount of at least one compound, stereoisomer thereof, an atropisomer thereof, a pharmaceutically acceptable salt thereof, a pharmaceutically acceptable salt of the stereoisomer thereof or a pharmaceutically acceptable salt of the atropisomer thereof according to claim 1.

14. The method according to claim 13, wherein, the cancer is selected from blood cancer, pancreatic cancer, colon cancer, rectal cancer, colorectal cancer or lung cancer.

15. The method according to claim 14, wherein, the blood cancer is selected from acute myeloid leukemia or acute lymphocytic leukemia; the lung cancer is selected from non-small cell lung cancer or small cell lung cancer.

16. The method according to claim 13, wherein the compound is:

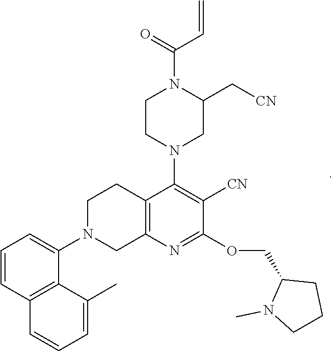 [SMILES: C=CC(=O)N1CCN(c2c(C#N)c(OC[C@@H]3CCCN3C)nc3c2CCN(c2cccc4cccc(C)c24)C3)CC1CC#N ; "."]

17. The method according to claim 13, wherein the compound is:

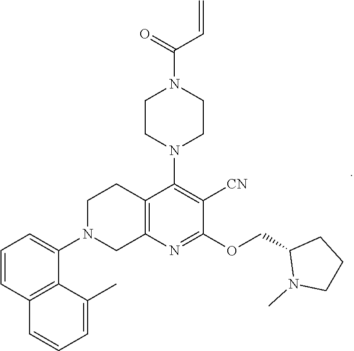 [SMILES: C=CC(=O)N1CCN(c2c(C#N)c(OC[C@@H]3CCCN3C)nc3c2CCN(c2cccc4cccc(C)c24)C3)CC1 ; "."]

18. The method according to claim 13, wherein the compound is:

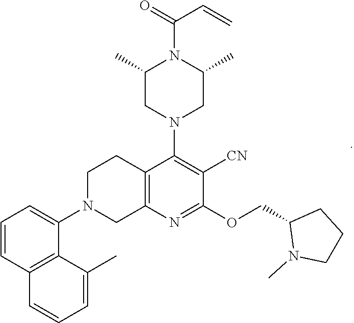 [SMILES: C=CC(=O)N1[C@H](C)CN(c2c(C#N)c(OC[C@@H]3CCCN3C)nc3c2CCN(c2cccc4cccc(C)c24)C3)C[C@@H]1C ; "."]

19. The method according to claim 13, wherein the compound is:

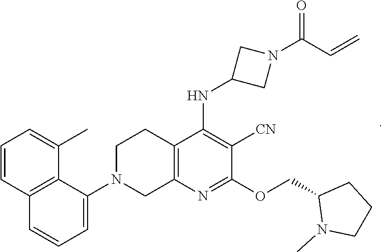 [SMILES: C=CC(=O)N1CC(Nc2c(C#N)c(OC[C@@H]3CCCN3C)nc3c2CCN(c2cccc4cccc(C)c24)C3)C1 ; "."]

20. The method according to claim 13, wherein the compound is: 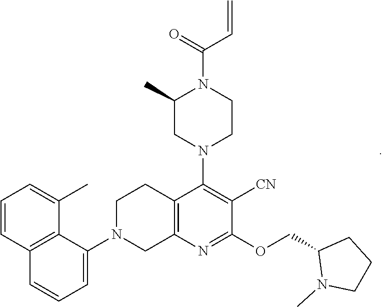 [SMILES: C=CC(=O)N1CCN(c2c(C#N)c(OC[C@@H]3CCCN3C)nc3c2CCN(c2cccc4cccc(C)c24)C3)C[C@H]1C ; "."]
